SEQUENCE LISTING (247 sequences)

SEQ ID NO: 1 - protein, 909 aa, Solanum lycopersicum
MAEEETNGAAAEGLPPPPPVPPDFTPAKTEIEPVKKKILRVPMARRGVGNKGQKIQILTNHFKVNVNNVDGHFFHYSVALFYEDGRPVDGKGVGRKVLDTVHETYDTELAGKDFAYDGEKSLFTIGALPRNKMEFTVVLDDVISNRNNGNSSPGGHGSPNEADRKRLRRPYQSKTFKVEISFAAKIPMQAIANALRGQESENSQEALRVLDIILRQHAAKQGCLLVRQSFFHNDPKNFVDVGAGVLGCRGFHSSFRTTQSGLSLNIDVSTTMIIQPGPVVDFLIANQNAKDPFSLDWAKAKRVLKNLRVKTTPTNQEYKITGLSDRPCREQLFTLKQKGKDADGEVQTTEVTVFDYFVNHRNIELRYSADLPCINVGKPKRPTFFPIELCSLVSLQRYTKSLSTFQRSSLVEKSRQKPQERMQVLSNALKINQYDAEPLLRSCGISISNNFTQIEGRVLPPPKLKTGGDDFVPRNGRWNFNNKRLVDPTKIERWAVVNFSARCNVQGLVSDLIKCGKQKGIMVEDPFDVFEESPQVRRAPPLVRVEKMFEQVQSKLPGAPKFLLCLLPERKNCDVYGPWKRKNLAEYGIVTQCIAPTRVNDQYITNVLLKINAKLGGLNSMLTVEHSPAIPMVSKVPTIILGMDVSHGSPGQSDVPSIAAVVSSRQWPSISRYRASVRTQSPKVEMIDNLFKRTSDTEDDGIMREALLDFYVSSGKRKPEHIIIFRDGVSESQFSQVLNVELDQIIEACKFLDEKWSPKFVVIVAQKNHHTKFFQPNDPNNVPPGTIIDNKVCHPRNYDFYLCAHAGMIGTTRPTHYHVLYDELGFSADDLQELVHNLSYVYQRSTTAISVVAPICYAHLAATQMGQWMKFEDASETSSSHNGVTNAGPVSVPQLPKLEEKVSSSMFFC

SEQ ID NO: 2 - protein, 1326 aa, Solanum lycopersicum
MSSICPQNWKNDVFLSFRGKDTRKTFVGHLNYALKQKGIHTFKDDERLERGKTISPELVKAIEESRFAIVVFSKNYASSTWCLDELVKIMECKKELGQTVIPIFYDVDPSHVTKQSETFAKSFAIHEENLKDDVEKVLCWRDAFRQAGKIAGYDLPNGYDGYESNCIQHVVEGILSKLCQVISTIENDLVGMESRVQEVSSLLRMETLDVRFIGIWGMGGIGKTTIARAVFGKYWGQFEGVCFLDNVVEMQKTHGLQYLQGVLLSKILKVSITIASVYEGMRIIKERLRSMKVLIILDDVIQKDQLEMLVGGRDWFGIGSRILITTTDKHLLHNHVVDEVYSVNLMTRDEAIELFSLHAFKQRIPDKDFEELLNQVVQTAGFLPLALKILGSSLCGLDKIVWKCTVDRLKDMPDDVLAKLKISFDGLSPSDQRLFLDIGCFYRGKLRSSVGEMLESCDIGSTIRVLIEKSLLFVSPYDTIEMHDLIQEMAWHIVRQGDTRRSRIWLPEDIEDLFTGNLEAEAVEGLSIPRDYVRKRDISCYNINETFRKMKRLRVLEVRATNFCSIDPITHLPSSIRWLDWEGCPLNSLPQSFEPSKLLRLDILESTTLQKLWSISKGLDKLKALYLSYCEKLEEVPSFDIMPNLERVKLEGCNSLREVSPSFGVLMKLTSMELIDCPSLKKIPSCVEMGSLKSLKLSCLPKLRELPETKGMHRLLTLELTNCQYLEKLPNFDQMESLETLKLSCLPKVTIFPPPDGMHCLQDIVIEYVPIVELPASIGNLRSLKQLRLSHCKDLVSIPNSFCCLKNLRALLIYNCKRLIYLPEKIGDLKLLKKLVVSGTSISQIPTSVTDLRELSFLSFSRWSGYRQGATFLFPPSSGVSLPLRVLKLKKNRLCSAEHFPDVGGLSSLAHLDLTRNDESTNQLFHYLDITFCEKLALPELPPFIKELYACDPLILKSIPDFLTKYSDLYSVSFTRHIENKGELTDLLLFVLRMISVASQCEKRPPLSLFFPGDIKWKWFTYYRKEQTKRFSTQLDPRWYESKFIKGFVLCFRVPSVIGQNQKCSNDKSQRGSSRRFGCTKVTAKLVQRYNRQEHDVLQKKCLIVARQTFCSHGSKYSICFNYIPFVACEKGKKLLNEYCFFEASLEPDIATKWGLLLVYEKKIQQIDQSTIAVQCDVDSPNSDLFRECNDDQGHRKEDVSVKRRRLDLRQVMKDSCSTSIVPDQQMETPSSSAAQSFQHRDESCSSGQPQTTQIPQADDPRVDEAIDEATSCVVVDQPDVFDVSPDEHKDNSMTTGSGCSVVSQQLETQCTSGKSQTLRLFPDHS

SEQ ID NO: 3 - protein, 139 aa, Solanum lycopersicum
MGVVIIDGSTIRDFVNDEAAFTKSIDKGFTDLDLNNDGVLSRSELRKAFESLHLIESHFGVDVAATPEELTDLYDSIFEKFDCDHNGTVDREEFGNEMRKIMQAIADGLGSSPIQMALDDSEQSLIKQAADLEASKLPA

SEQ ID NO: 4 - protein, 303 aa, Solanum lycopersicum
MPTIAKHDGRQLETGLRITSLSTTQMQPWWHGYGDNTMPLASENAVAQEKSEGGNQDKETKALAMESGSDGNNEQYKQHLKHFAPTTAAIMAEQQKELTGHSAMLASYPYPDMQYGGMMTYGAPVHPHLFEIHHARMPLPLDMEEEPVYVNAKQYHGILRRRQIRAKAELERKAIKARKPYLHESRHQHAMRRARGTGGRFLNTKKLNDMDCTPTETQKYGATIATHSGNSSGSGSSDQGGKEGSTVQDMHKGHSQSFTTGNGHGSSVYFSASSGSEQGNGHYGHGSWSLLVNQASQGAASSN

SEQ ID NO: 5 - protein, 189 aa, Solanum lycopersicum
MVCIRKATIDDLLAMQACNLLCLPENYQMKYYFYHILSWPQLLYVAEDYNGKIVGYVLAKMEEETTECHGHITSLAVVRTHRKLGLATKLMTAAQNAMEQVFGAEYVSLHVRKSNRAAFKLYTETLGYKIHDMEAKYYADGEDAYDMRKELKGKKHHQHHHHHHHHGGGCCSGEVKVGPKEGAAETKAA

SEQ ID NO: 6 - protein, 387 aa, Solanum lycopersicum
MYCGSILTVSNFNYLILFLIIKLMEYWGSIEKPIVLLGCAYLSWKIVTHIWSFMNIEKDPITVLVTGAAGQIGYALVPMIARGAMLGLDQPVIIHMLDIEPAAESLKGVKMELIDAAFPLLKDVVATTNVVEACKGVNVAVMVGGFPRKEGMERKDVMTKNVSIYKAQASALEQHAAPDCKVLVVANPANTNALILKEFAPSFPAKNITCLTRLDHNRALGQISEKLNVHVGDVKNVAIWGNHSSTQYPDVNHATVKTAAGEKPVRELVANDQWLNTEFITTVQQRGAAIIKARKLSSALSAASSACDHIHDWVLGTPKGTWVSMGVYSDGSYGIPAGLIYSFPVTCEKGEWSIVQGLKIDEFSRAKMDATAKELAEEKSLAYSCLN

SEQ ID NO: 7 - protein, 475 aa, Solanum lycopersicum
MNVMRRLKSIASGRSSVSDPGGDSSIKRVKVEKEVDQRVVGETQMEEGCTTTTVPKEDMASTSMETTAGSTSTMDIRLENSELDELPKEMHEMKIKDEKDDKADSLDDNLKDMEPAVVSGNGTETGQIIVTTVSGRNGQEKQTLSYMAERVVGTGSFGVVFQAKCLETGESVAIKKVLQDRRYKNRELQIMRTLDHPNVVKLRHCFYSTTEKNEVYLNLVLEYVSETVYRVSRHYSRMNQHMPNIYVQLYTYQICRALNYMHGVLGVCHRDIKPQNLLVNPHSHQLKLCDFGSAKMLVPGEPNISYICSRYYRAPELIFGATEYTTAIDMWSAGCVMAELLLGQPLFPGESGVDQLVEIIKILGTPTREEIRCMNPNYTEFKFPQIKAHPWHKIFHKRMPPEAVDLVSRLLQYSPTLRCTALEACAHPFFDSLREPNACLPNGRPLPPLFNFSPQELSGVPAELRKRLIPEHLRK

SEQ ID NO: 8 - protein, 144 aa, Solanum lycopersicum
MPKNKGKGGKNRKRGKNEADDEKRELVFKEDGQEYAQVLRMLGNGRCEAMCIDGTKRLCHIRGKMHKKVWIAAGDIILVGLRDYQDDKADVILKYMPDEARLLKAYGELPENTRLNEGIANLDEEDENAADDYIEFEDEDIDKI

SEQ ID NO: 9 - protein, 186 aa, Solanum lycopersicum
MESQFIERYHSHQPSEHQCSSSLVKHIKAPVDIVWSLVRRFDQPQKYKPFISRCTVKGDLTIGSVREVNVKSGLPATTSTERLELLDDEEHILGIRIVGGDHRLKNYSSVITVHPETLDGRPGTLVIESFVVDVPEGNTKEETCYFVKALINCNLKSLADVSERMAMQGGVLPVSVNWSSSNQIEI

SEQ ID NO: 10 - protein, 123 aa, Solanum lycopersicum
MAAYAAMKPTKPGLEEPAEMVHKIRITLSSKNVKNLEKVCADLVRGAKDKRLRVKGPVRMPTKVLNITTRKSPCGEGTNTWDRFELRVHKRVIDLFSSPDVVKQITSITIEPGVEVEVTIAES

SEQ ID NO: 11 - protein, 206 aa, Solanum lycopersicum
MDGGSSNAPVQQLTAEAAAQLQEGIGLVLTRWTALQMAIENEWGGRDTREKSNQLNVDIFSAFTQSKEKVYMDDIEEILDEFMISLNTEVNDGSLEEVAEKLMYMHEECLEGDFNSIKVLRETNVGRRPATYVRQDASDDDDSSNDGDDRNENFGNNSSDMAIDSMESQPSLGQDMIVEPVRKQPAEVDPDGWTTVSTKRNKGRRN

SEQ ID NO: 12 - protein, 316 aa, Solanum lycopersicum
MAEANSYAALTKAFSGLGVDENLFISTLGKWNRHQRESYRVSTPGFFREDKRQFQRWDDQHILQLRQEFLRLKDAVVLYTMHPWERDARLFKEALLLQVPKHDVLIEIACTRSSEDLLGARKAYHSLFEHSIEEDIAFHVRSSERKLLVALVSSYRYEGPRVNDDLAKSEAKIFVNAIKNANKKKLIDEDEEIVRILSTRSKLHLKAIYSHYKEITGNFLDEDLECDLTLKQVVQCLCIPKAYFSKILIASLRLDVDESAKDSVTRVIVSRADDDDMQQIKEEFQSKYGTTLAEKIAEVANGSYKDFLLTIIAKSE

SEQ ID NO: 13 - protein, 210 aa, Solanum lycopersicum
MGLSFGKLFSRLFAKKEMRILMVGLDAAGKTTILYKLKLGEIVTTIPTIGFNVETVEYKNISFTVWDVGGQDKIRPLWRHYFQNTQGLIFVVDSNDRDRVVEARDELHRMLNEDELRDAVLLVFANKQDLPNAMNAAEITDKLGLHSLRQRHWTGTFRAHVLPLEKGCMRGLTGSQTTLQTRVEHEIHDFCSYIFVNIDFKQCLEHLCTC

SEQ ID NO: 14 - protein, 393 aa, Solanum lycopersicum
METFLFTSESVNEGHPDKLCDQISDAVLDACLEQDPESKVACETCTKTNLVMVFGEITTKAIVDYEKIVRDTCRNIGFVSDDVGLDADNCKVLVYIEQQSPDIAQGVHGHLTKRPEEIGAGDQGHMFGYATDETPELMPLSHVLATKLGARLTEVRKNGTCAWLRPDGKTQVTVEYSNDNGAMVPIRVHTVLISTQHDETVTNDEIARDLKEHVIKPVIPEKYLDENTIFHLNPSGRFVIGGPHGDAGLTGRKIIIDTYGGWGAHGGGAFSGKDPTKVDRSGAYIVRQAAKSIVASGLARRCIVQVSYAIGVPEPLSVFVDTYGTGKIPDREILKIVKENFDFRPGMMSINLDLKRGGNRRFLKTAAYGHFGRDDPDFTWEVVKPLKWEKPQD

SEQ ID NO: 15 - protein, 601 aa, Solanum lycopersicum
MALKLSAAEAAKVAIQSIGCGYDISLDLRLKYCKGHPDNSRLIEIDENESGEVVLPGGISIPNVSKSIKCDKGEHTRFGSDVLSFQQMSEQFNQEVSVNGKIPSGLFNTMFEFSGSWQKDAAYTKTLAFDGVFITLYSVALEKSQMVLCDYVKKEVPSTWDPAALARFIEKFGTHVIVGIKMGGKDVIYMKQQHSSSLLPADVQKRLKTMADKRFSGANEQGGVESQQTHQNEKFEIREHRLRFADPNISGSYAHKEDTVCICKRRGGSDSRGLMHNQWLQSVDQEPDVISMSFIPITSLLNGISGSGFLSHAINLYLRYKPPIEELHQFLEFQLPRQWAPVFGDLPLGPQRRHLSFPSLQFSLLGPRLYVNTTPVDVGKRPVTGLRLYLEGRRSNRLAVHLQHLSSLPKVFQLEDDPKGNFRRESYDRKYYEKVQWKNFSHVCTAPVESDEDLNIVTGAQLELGDYGFKKVLFLRLRFSTVMGATMVKHAEWDGSPGLARKSGLISTLISQHFTSVQKPPPRPDDVNINSAIYPGGPPVPVQAPKLLKFVDTTEMTRGPQEPPGYWVVSGARLMVEKGRISVRVKYSLLTVIQPDDEISE

SEQ ID NO: 16 - protein, 332 aa, Solanum lycopersicum
MAYYNYNLINKMVTIIFILVLVIVDVTMVFGQGTRVGFYSSTCPRAESIVQSTVRSHFQSDPTVAPGLLRMHFHDCFVQGCDGSILISGTGTERTAPPNSNLRGFEVIDDAKQQIEAVCPGVVSCADILALAARDSVLVTKGLTWSVPTGRRDGRVSSASDTSNLPGFTESVDAQKQKFAAKGLNTQDLVTLVGGHTIGTSACQFFSYRLYNFNSTGGPDPSIDATFLSQLQALCPQNGDGSKRVALDTGSVNNFDTSYFSNLRNGRGILESDQILWTDASTKVFVQRYLGLRGFLGLRFGLEFGKSMVKMSNIEVLTGTNGEIRKVCSAFN

SEQ ID NO: 17 - protein, 353 aa, Solanum lycopersicum
MLANQKSKAVTYRKLARSYHPDVNKEPGAEQKFKEISNAYEVLSDDEKRSIYDKYGEAGLKGAGAGMGDFSNAFDLFESLFDGFGGMGGMGGMGGRGSRSRATEGEDQGYNLVLNFKEAVFGVEKEIEISRLETCGTCDGSGAKPGTKPSTCNTCGGQGQVVSSARTPLGVFQQVTTCSSCGGTGEISTPCGTCNGDGRVRKSKRISLKVPPGVDSGSRLRVRSEGNAGRRGGPPGDLFVMIEVLPDPVLKRDDTNILYNCKVSYIDAILGTTMKVPTVDGMVDLKIPAGTQPGTTLVMAKKGVPFLSKPNMRGDQLVRVQVEIPKRLSSEERKLIEELANLNKPKAATNSKR

SEQ ID NO: 18 - protein, 175 aa, Solanum lycopersicum
MADVEYRCFVGGLAWATTDQTLSEAFSQYGEVVESKIINDRETGRSRGFGFVTFKDEQAMRDAIEGMNGQDLDGRNITVNEAQSRGGGGGGGGRGGGGYGGGRREGGGGGYGGGGGYGGGRREGGGGGYGGGGYGGGRREGGYGGGGGGYGGGDRYNDRSSRGGGGGGSDGNWRN

SEQ ID NO: 19 - protein, 356 aa, Solanum lycopersicum
MDFSYSSPSHFILSLCFALLILSRGVLGATFTFVNKCEYTVWPGILANAGSPTLQSTGFELPQESSRVFTAPTGWSGRFWGRTSCTFEGSGSGSCGTGDCGSGEVECNGAGAAPPATLAEFTLGTVGQDFYDVSLVDGYNLPMVVEASGGSGMCATTGCVTDLNQICPSELRSGGGEACRSACEAFNKPEYCCSGAYNTPSTCKPSMYSQMFKSACPRSYSYAYDDPTSTFTCSGADYTITFCPSTPSQKSTKDTTTPNGDGSYGATGTGVVDPATGGIESSSGQMGTGSGTGFEFGAGTGYGSGTETQPGSGSQAMLADGSYLAGLAMGESSRAFFPLNLAGSALATTLVSLVFL

SEQ ID NO: 20 - protein, 249 aa, Solanum lycopersicum
MARYDRAITVFSPDGHLFQVEYAMEAVRKGNAAVGVRGTDTVVLGVEKKSTPKLQDSRSVRKIVNLDDHIALACAGLKADARVLVNKARIECQSHRLTVEDPVTVEYITRYIAGLQQKYTQSGGVRPFGLSTLIIGFDPHTGVPSLYQTDPSGTFSAWKANATGRNSNSTREFLEKNYKETSGQETVKLAIRALLEVVESGGKNIEVAVMTKEHGLKQLEEAEIDAIVAEIEAEKAAAEAAKKAPPKET

SEQ ID NO: 21 - protein, 539 aa, Solanum lycopersicum
MGFVSTILGFCGFGVGVSCGLTIGYYLFIYFQPCDVKDPVIRPLVERDSKSLQQLLSEIPLWVKCPDYDRVDWLNKFIEYMWPYLDKAICRTAKDIAAPIIAEQIPKYKIDSVEFETLTLGSLPPTFQGMKVYVTEEKELIMEPSIKWAGNPNVTVAVKAFGLKATVQVVDLQVFAAPRITLKPLVPSFPCFANIFVSLMEKPHVDFGLKLLGADLMSIPGLYRFVQETIKDQVANMYLWPKSLEVQILDPSKAMKKPVGVLHVKILRAMNLKKKDLLGASDPYVKLKLTESKLPSKKTPVRHKNLNPEWNEEFNMVVKDPESQALELSVYDWEKIGKHDKMGMNVIPLKDLTPDETKTMTLSLLKNMDANDSQNDKDRGQIMVELTYKPFKEDELPKDFEDNDAAHKVPEGTPPGGGVLMIIVHEAQDVEGKHHTNPYVKILFKGEERKTKQVKKNRDPRWEEEFTFVLEEPPVNDRVHMEVVSTSTRIGLLHPKETLGYVDINLSDVVSNKRINEKYHLIDSKNGRLQVELQWRTAS

SEQ ID NO: 22 - protein, 261 aa, Solanum lycopersicum
MSLFFLVLLLFVSSVTACDRCVHRSKVAYFSSSSALLSGACGYGNLAIGFNGGRLAAAVPKLYDNGAGCGACYQIRCTDPKICSKHGTTVMVTDLNTNNQTDFVISSRAFMAMAIEGKAQDVHKLGIADVEYKRVPCDYKGKNLAVRVDESSQKPHYLAISIFYQGGQTEIVSVDVAQVGSSNWNFLSRNHGAIWDTSRVPSGALQFRFVVTAGYDGKWIWAKSVLPSDWKNGVVYDTGLQITDIAQEGCSPCDDGSWKLN

SEQ ID NO: 23 - protein, 629 aa, Solanum lycopersicum
MACMKLGSKTDAFQRKGQAWFCTTGLPSDIVVEVGEMTFHLHKFPLLSRSGVMEKRIAEASEGEDGCVIELNDIPGGAKTFELVAKFCYGVKLELTAGNAVYLRCAAEHLDMTEEYGEGNLISQTEIFLNQVVLRSWKDSLKALQTCDDVLPYAEELRITKRCIDSLAVKACTDPNLFGWPVMEHVGPLQSPGGSILWNGISTGARPKHSSSDWWYEDASTLSLPLYKRLISAMESQGVKQDIVAGSLSYYAKKYLPGLNRRQTSSESTNRLAPVGLGSSLSEEDQKLLLEEVDNLLPMQKGLVPTKFLFGLLKTALILRACPSCISNLEKRIGMQLDQATLEDLLMPNFSYSMETLYNVDCVQRILEHFLAMDQGTGGESPCSIDDEQLIGSPSLTPITMVAKLIDGYLAEVAPDVNLKLPKFQTLAASVPEYARPLDDGLYRAIDIYLKSHPWLGESDREQLCRLMDCQKLSLEACTHAAQNERLPLRIIVQVLFFEQLQLRTSIAGCFLVSENLDEGSRQLRSGTVGPNEGGWATAVRENQVLKVGMDSMRVRVSELEKECSTMRQEIEKLGRSKGSSTWGNVSKKFGFKMKSQMCSAQEGSVSNQNKINSKAIKDDKAKENYRKH

SEQ ID NO: 24 - protein, 542 aa, Solanum lycopersicum
FFYGPLWPKKVHYWLAVADSVEGKHQRKMDGTAVQGTSSVDSFLRNYKLGKTLGIGSFGKVKIAEHTLTGHKVAVKILNRRKIRNMDMEEKVRREIKILRLFMHPHIIRLYEVIETPSDIYVVMEYVKSGELFDYIVEKGRLQEDEARNFFQQIISGVEYCHRNMVVHRDLKPENLLLDSKWNVKIADFGLSNIMRDGHFLKTSCGSPNYAAPEVISGKLYAGPEVDVWSCGVILYALLCGTLPFDDENIPNLFKKIKGGIYTLPSHLSAGARDLIPRMLIVDPMKRMTIPEIRLHPWFQAHLPRYLAVPPPDTTQQAKKIDEEILQEVVKMGFDRNNLTESLRNRVQNEGTVAYYLLLDNRHRVSTGYLGAEFQESMEYGYNRINSNETAASPVGQRFPGIMDYQQAGARQFPIERKWALGLQSRAHPREIMTEVLKALQELNVCWKKIGQYNMKCRWVPSLPGHHEGMGVNSMHGNQFFGDDSSIIENDGATKLTNVVKFEVQLYKTREEKYLLDLQRLQGPQFLFLDLCAAFLAQLRVL

SEQ ID NO: 25 - protein, 305 aa, Solanum lycopersicum
MSSNIIHDADGSASRIVLKEHTGSTAEVLFQGGQVVSWKNERREELLFRSNKTLWKPPKAFRGGIPISFPQFGTFGSLERHGFARNRVWELDDSPSPLPAANNQSTVDLILKSTEEDLKTWPHRFEVRLRISLSAGKLTLIPRVRNIDNKPFSFTFSLRNYLSVSDISEVRVEGLETLDYFDNLQRGERYTEQADAITFDSETDRVYLSTPTKTAIIDHEKKRTYVLQKEGMADAVVWNPWDKKAKSLPDLGDQDYKKMLCVDSAAVETPIILKPSEEWRGRQELSTVSSSYCSGQLDPRKVIYG

SEQ ID NO: 26 - protein, 204 aa, Solanum lycopersicum
MEMQSEFDRLLFFEHARKSAETAYAQNPLDADNLTRWGGALLELSQFQPVAESKQMISDATSKLEEALTVNPEKHDALWCLGNAHTSHAFLTPDMDEAKVYFEKATQCFQQAFDADPSNDLYRKSLEVTAKAPELHMEIHRHGPMQQPMAAEPSTSTSTKSSKKKKSSDLKYDIFGWVILAVGIVAWVGFAKSNMPPPPPPHPQ

SEQ ID NO: 27 - protein, 352 aa, Solanum lycopersicum
MKHIATFLLLVSVSTLVLQVVAENPISQAKAESAILQDSIVKQVNENEKAGWRAALNPQFSNFTVSQFKRLLGVKPTRKGDLKGIPILTHPKLLKLPQEFDARVAWPQCSTIGRILDQGHCGSCWAFGAAESLSDRFCIHYGLNISLSANDIIACCGYLCGDGCDGGYPLEAWKYFVRKGVVTEECDPYFDNKGCSHPGCEPGYPTPQCKRKCVKENLLWSKSKHFGINAYLINSDPYSIMTEVYKNGPVEVSFTVYEDFAHYKSGVYKHINGEEMGGHAVKLIGWGTSEDGEDYWLLANQWNRGWGDDGYFKIRRGTNECGIEEEVVAGMPSAKNLNVELDVSDALLDASM

SEQ ID NO: 28 - protein, 354 aa, Solanum lycopersicum
MALTLKSLITPLLFGAFFILILQVAAEKPITEAKLESAILQDSIVKQVNENAEAGWKAAFNPQLSNFTVSQFKRLLGVKPAREGDLEGIPVLTHPKLKELPKEFDARKAWPQCSTIGRILDQGHCGSCWAFGAVESLSDRFCIHYNLSISLSVNDLLACCGFLCGSGCDGGYPIAAWRYFKRRGVVTEECDPYFDTTGCSHPGCEPLYPTPKCHRKCVKGNVLWRKSKHYGVNAYRVSHDPQSIMAEVYKNGPVEVSFTVYEDFAHYKSGVYKHVTGGNMGGHAVKLIGWGTSEQGEDYWLIANSWNRGWGEDGYFKIRRGTNECGIEHSVVAGLPSARNLNVELGDAVLDASM

SEQ ID NO: 29 - protein, 341 aa, Solanum lycopersicum
MGFSVFLFTFLLQHLLIFNHVFYLCSTDRCSNRFFTLPRLISLSLNLNMAEASAPAVPSTELLEWPKKDKRRMLHAVYRVGDLERTIKFYTECFGMKLLRQRDIPEEKYSNAFLGFGPEESHFVVELTYNYGVDKYDIGTGFGHFAIATPDVYKLVEEIKAKGGTVTREPGPVKGGSSVIAFVKDPDGYLFEIIQRESTPEPLCQVMLRVGDLERAIKFYEKALGMQVVKKVDRPEHKYTIAMMGYAPEKETTVIELTYNYGVTEYTKGNAYAQIAIGTDDVYKSAKVVNLAIQELGGKITRQPGSIPGLNTKITSFLDPDGWKTVLVDNKDFLKELESSK

SEQ ID NO: 30 - protein, 264 aa, Solanum lycopersicum
MLLLSLLVAFALFSPTPFVHSHYNWSPSSSSTSNFQSEWRPARATYYAAADPRDVVGGACGYGDLERNGYGKATAGLSTVLFDKGQICGACYEVRCVEDLRWCIPGTSIIVTATNFCAPNYGFDLDGGGHCNPPNAHFVLPIEAFEKIAIWKASNMPIQYRRIKCRKEGGVRFTLGGSGIFLSVLISNVAGSGDIVAAKVKGSRTGWLPMGRNWGQNWHINADLKNQPLSFEMTSGDGATLTSYNVAPKNWNFGQTFEGKQFGS

SEQ ID NO: 31 - protein, 845 aa, Solanum lycopersicum
MEVNSLQKWVLLWCIVLFISSGLVHCDVTYDRKAIVINGQRRLLFSGSIHYPRSTPEMWEDLINKAKEGGLDVVETYVFWNVHEPSPGNYNFEGRYDLVRFVKTIQKAGLYAHLRIGPYVCAEWNFGGFPVWLKYVPGISFRADNEPFKNAMKGYAEKIVNLMKSHNLFESQGGPIILSQIENEYGPQAKVLGAPGHQYSTWAANMAVGLDTGVPWVMCKEEDAPDPVINTCNGFYCDNFFPNKPYKPAIWTEAWSGWFSEFGGPLHQRPVQDLAFAVAQFIQRGGSFVNYYMYHGGTNFGRTAGGPFITTSYDYDAPIDEYGLIRQPKYGHLKELHRAVKMCEKSIVSADPAITSLGNLQQAYVYSSETGGCAAFLSNNDWKSAARVMFNNMHYNLPPWSISILPDCRNVVFNTAKVGVQTSKMEMLPTNSEMLSWETYSEDISALDDSSSIRSFGLLEQINVTRDTSDYLWYITSVDIGSTESFLHGGELPTLIVETTGHAMHVFINGQLSGSAFGTRKNRRFVFKGKVNLRAGSNRIALLSVAVGLPNIGGHFETWSTGVLGPVAIQGLDHGKWDLSWAKWTYQVGLKGEAMNLVSTNGISAVDWMQGSLIAQKQQPLTWHKAYFNTPEGDEPLALDMSSMGKGQVWINGQSIGRYWTAYATGDCNGCQYSGVFRPPKCQLGCGEPTQKWYHVPRSWLKPTQNLLVLFEELGGDPTRISLVKRSVTNVCSNVAEYHPNIKNWQIENYGKTEEFHLPKVRIHCAPGQSISSIKFASFGTPLGTCGSFKQGTCHAPDSHAVVEKKCLGRQTCAVTISNSNFGEDPCPNVLKRLSVEAHCTPTQN

SEQ ID NO: 32 - protein, 433 aa, Solanum lycopersicum
MSTLICLASPSTLVLSISKLKNPKTASLSVLCWTKEKKSLMNSKELHSAGALTLKRSMIILRGVKKKTQPSQKKSYIITFNKQHREIVIGKYLKHVIEEGRAIEFKNKKQKIYCNDRMPSWWACYNSMWKHINFEHPATFDTLAMDPKKKAEIIDDLIALTNGKDYYSKVGKAWKRGYLLYGPPGTGKSTMIAAIANYLNYDIYDLELTSVKDNLELKKLLMEITSKSIIVIEDIDCSVDLTGKRKKAKKNKKKDDSDSDSDSDSDSDSDSDYEGKKKLTLSGLLNFIDGIWSACGEERIIIFTTNHVDKLDPALIRRGRMDMHIEMSYCRYEAFKVLAKNYLGIDTHPLFQEIQPLLEEVNLSPCDVAETLILKNTSGGIEICLNNLILALKDSKEKAIQEAREKEKQDSEEKPKKNKKLKKIYGRFKKLFR

SEQ ID NO: 33 - protein, 353 aa, Solanum lycopersicum
MSLKINLLICFFILHLWAATGHKIGIYEIKKGDFSVKVTNYGARIISVLLPDKNGKIGDIVLGYDTIGEYVNDTSSFGAIVGRVVNRIAGARFTLNGTLYKLIANDGKNTIHGGPKGFSHVVWKVSKYKKDGPHPHITLTYHSTDGEQGFPGDLLASVTYALEDPYKLSVVMKAKALNKATPVNLAQHTYWNLGGHNSGDVLSNDIQIFASHITPLDQHFIPTGKIAPVKNTPYDFLKPRKIGSQFKKLQNGYDINYPLDSTEKLKPTATVHDNKSGRVMSLQTTAPAVQLYTANTLVKKGKGGFVYQPYSAVCLETQGFPDAVNHPNFPSTIVTPEHPYFHKMLFTFSIKKY

SEQ ID NO: 34 - protein, 410 aa, Solanum lycopersicum
MTTVVPTSEEDPVLSVVRFTAEMSWADAGAETAEPEVNRLCVEAQKCMIEGRWLDLVSLMLASADIIFSKASEKDLECVFTIMCNLVKKPESLDQVHEMAELISAKIIQQPNDKPALRLKILFNLYNLLENPYSRFCVYLKSLKLATAGKVTEHILSSIKMMDNFLKEWNLGVKDQRELFLAISNILKESKGSTKDSFMFLTKYLETFSSEDASNMTEAKEEAASAIVDFVRSPDMFKCDLLDMPAIVQLEKDGQYGPVYQLLKIFLTQRLDAYLEFEAANSALLKTYGLVHDDCISKMRLMSLVDLGLNESSQIPYSVIKQALQIDDIEVESWVVKAITAKLLDCKIDQMNQVVIVSRCTERVFGVHQWQDLRTKLVTWRGNIAGVISTVQANKITEDSTQAVQGLAIR

SEQ ID NO: 35 - protein, 366 aa, Solanum lycopersicum
MLSSCFGSCFGGDDGDDLLWHMDLKPHATGEYSVAVVQANSLLEDQGQVFTTPSATYVGVYDGHGGPQASRFINNNLFPQLQKLALEEGGLSEDVIRKAFDAIEEGFLRLVKQSWLDQPQIASAGSCCLFGAISKDELYVANLGDSRAVLGRRAETGQTGDSAVVVAERLSIDHNVGVEEVRKEVEALHPDDSHVVVYVRGVWRIKGIIQVSRSIGDVYLKKPEFNRDPMFIQYGYPIPLKRAVMSAEPSILIRKIRPEDLFLIFASDGLWDQLTDDEAKDIVLKNPRIGIAKRLVRAALEGAAEKKEMRYEDIKRLEKGERRHVHDDITVIVIYLDHPGRSSNSGINIVKGTNVPLDIYSLNSGH

SEQ ID NO: 36 - protein, 356 aa, Solanum lycopersicum
MDGKENNGVNGSNKVGIPKCCLKARVFDPELEANCHSTVVSGWFSQPPTSFDGKEKVLYFNNPMWPGEAHSLKVEKVLFKGKSEFQEVLVFESASYGKVLVLDGIVQLTEKDECAYQEMIAHLPLCSIKSPKKVLVVGGGDGGVLREISRHSSVELIDICEIDKMVIDVSKKYFPDLAIGFEDPRVNLHVGDAVEFLKNTPEGKYDAIIVDSSDPVGPAIELVEGPFFAKIARALRPGGVLCNMAESMWLHTHLIQDMISICRETFSSVHYAWASVPTYPSGVIGFLLCSTEGPFVDFKHPVNPIEKLEGALQHQRELKFYNSEMHEAAFALPCFLRREVSGLRDSPRSAGSGVKI

SEQ ID NO: 37 - protein, 292 aa, Solanum lycopersicum
MGEETKQEPPKEEVKTEEEQTEPKPPSPFVLYVDLHCVGCAKKIERSISKIRGVEGVVIEMAKNTVTIKGVIEPQAVCDRITKKTKRVAKVLSPLPAAEGEPIPQVVASQVSGLTTVELNVNMHCEACAEQLKKKILRMKGVRSAETEASTGKVTVTGTMDANKLVDYVYRRTKKQAKIVPQPEPEPEKPVEETKPDEAKPVEEPTPEDKKDEGTPPTENKEGGDGEKPPEGEKNEGGGGGEAMIIPIEDHYDEHTINKLMYYHPYQPLYVIERIPPPQLFSDENPNACCIT

SEQ ID NO: 38 - protein, 337 aa, Solanum lycopersicum
MAKVKIGINGFGRIGRLVARVALQRNDVELVAVNDPFISVDYMTYMFKYDSVHGQWKHHELKVKDEKTLLFGEKAVTVFGFRNPEEIPWAQTGADYIVESTGVFTDKDKAAAHLKGGAKKVIISAPSKDAPMFVVGVNEKEYKPELNIVSNASCTTNCLAPLAKVINDRFGIVEGLMTTVHSITATQKTVDGPSAKDWRGGRAASFNIIPSSTGAAKAVGKVLPALNGKLTGMAFRVPTVDVSVVDLTVRLEKEATYDEIKAAIKEESEGKLKGILGYTEDDVVSTDFVGDNRSSIFDAKAGIALSKNFVKLVAWYDNEWGYSTRVVDLIIHMSSVQ

SEQ ID NO: 39 - protein, 319 aa, Solanum lycopersicum
MGYANGLLCPLGKQPLLSFGVISDVQYADIDDGTSFLGVPRYYRHSVSVLQRAVKKWNQEKPKFVLNFGDIVDGYCPKDQSMIAVKKIVDEFDKFNGTVYHMIGNHCLYNLPRKDLLPLLRIPGHDDHAYFDFSPIPEYRFVILDAYDISAIGWPEDHPNTLKALKVLQEKNPNSDKNSPSGLVGPARRFLMFNGGVGKEQLEWLDHVLQEATKLNQNVIVCCHLPLDPGASSLAALLWNYDEVMDVIHRYSCVKVCMGGHDHKGGQSVDSHGVHHRVLEAALECPPGTDSFGHIDAFDDRLLLFGTDRMKSTEMVFRH

SEQ ID NO: 40 - protein, 410 aa, Solanum lycopersicum
MNTSSSIIFKFVAFLVIFSSFSTHVLSTLQANQTFRPKEELLKLKKIKGYLKKINKRPVRTIKSPDGDLIDCVLSHLQPAFDHPQLKGMKPLEPPKRPKANESMNIMEESLQLWTVSGESCPEGTVPIRRTTEKDVLRSNSLRRFGRKIRRGVRHDTSSNDHEHAVAFVNGEQYYGAKASINVWTPRVTDQYEFSLSQLWVISGSFGNDLNTIEAGWQVSPELYGDNYPRFFTYWTTDAYQATGCYNLLCSGFVQINNRIAMGAAISPRSSVNGRQFDIGIMIWKDPKHGHWWLEFGSGLLVGYWPDFLFSHLRGHASMVQFGGEVVNSRSMGYHTSTQMGSGHFADEGFGKASYFRNLQVVDWDNNLIPLSNLHLLADHPNCYDIRAGKNNVWGNYFYYGGPGRSSRCP

SEQ ID NO: 41 - protein, 363 aa, Solanum lycopersicum
MRRIHLYIAIAYVVIFQTSCSAHFFPNISSIPSSLLKPNATAWTSFQKLLGCQPGQKVDGIAKIKKYFQHFGYINNLTSFNFTDEFDDTLESALKTYQRNFNLKATGVLDAPTIQHLIKPRCGNADLVNGTSTMNAGKPHTVAHYSFFPGRPKWPEGKTDLTYAFLPANNLTDDIKSVFSRAFDRWSEVTPLSFTEIPSFQSADIKIGFLTGDHNDGEPFDGPMGTLAHAFSPPAGHFHLDGEENWVVDGVPVNEGNFFSILSAVDLESVAVHEIGHLLGLGHSSVEDSIMYPSLESGIRRVELVEDDIKGVQELYGSNPNYTGTNTTLTPSGLDNDTNGAPIRSSVWIHGFLLVVGFFIYSI

SEQ ID NO: 42 - protein, 356 aa, Solanum lycopersicum
MAHLSDLVNLNLSDSSEKIIAEYIWIGGSGMDVRSKARTLSGPVDDPSKLPKWNYDGSSTGQAPGEDSEVILYPQAIFKDPFRRGNNILVICDCYTPAGEPIPTNKRHNAAKIFSNPNVVVEEPWYGLEQEYTLLQKEINWPLGWPIGGFPGPQGPYYCGIGCGKAFGRDIVDAHYKACIYAGINISGINGEVMPGQWEFQVGPSVGIASGDELWAARYILERITEIAGVVVSFDPKPIPGDWNGAGAHTNYSTKSMRNEGGYEVIKKAIEKLGLRHKEHIAAYGEGNERRLTGRHETADINTFKWGVANRGASIRVGRDTEKEGKGYFEDRRPASNMDPYVVTSMIAETTILWNP

SEQ ID NO: 43 - protein, 598 aa, Solanum lycopersicum
MCTKFVKEMEKMALYVVVLLIICYASPARSDGSDHKYKSGDQVPLYANKVGPFHNPSETYRFFDLPFCTPDHVKEKKEALGEVLNGDRLVSAPYKLDFLYDKDSEIVCKKKLSKEEVSQFRSAVAKDYYFQMYYDDLPIWGFLGKVDKEGKSDPSEYKYYLFKHLHFEIFYNDDRVIEINARTDPNALVDITEDKEVDVDFMYTVKWKETKTPFDKRMEKYSQSSSLPHHLEIHWFSIINSCVTVLLLTGFLATILMRVLKNDFVKYAHDEEAADDQEETGWKYIHGDVFRYPSHKSLMAAALGSGTQLFTLTIFIFLLSLVGVFYPYNRGALFTALVVIYALTSGIAGYTAASFYCQLEGTNWVRNLILTGALFCGPLFLTFCFLNSVAIAYSATAALPFGTIVVIFLIWALVTSPLLVLGGIAGKNSRAEFQAPCRTTKYPREIPPLPWYRGTLPQMAMAGFLPFSAIYIELYYIFASVWGHRIYTIYSILFIVFIILIIVTAFITVALTYFQLAAEDHEWWWRAFLCGGSTGLFIYGYCLYYYYARSDMSGFMQTSFFFGYMACICYGFFLMLGTVGFRAALFFVRHIYRSIKCE

SEQ ID NO: 44 - protein, 297 aa, Solanum lycopersicum
MAVERFACGDELPEGCIATALSLTSPKDACRLALVASTFRSAAESDAVWERFLPPDYYDIISRSIDGPDSLNFGSKKDLYLYLCDNPILIDGGTRSFSLEKGSGKKCYMLAARSLQIVWVDTPRHWRWISLPESRFSEVAELLDVCWFDISGKINTNMLSPDTTYGAYLVFTTKTRTCGFENQPAESSVGIAGHERYTHTFYLNPQGGRRQYRYQVVPRRLGIFTHHMARILREETEDAPKGCRPKQRSDGWMEIELGEFFVKRGQEAEVEISLTEVKGGDRKAGLIIEGIDIRPKE

SEQ ID NO: 45 - protein, 348 aa, Solanum lycopersicum
MVSTTLSSFLLVLVLSFFVSEAQRPALTKGLSWSFYQSSCPQLESIIRNRLQKQIKDDVGQAAGLLRLHFHDCFVQGCDGSVLLDGSAGGPSEQTAIPNLTLRKRSFKIIDDLRKRIQDECGQVVSCSDIVAIAARDSVVLTGGPNYDVPLGRKDGVNFATEQATIDNLVAPSANTTTVLSLLATKGLDATDAVALSGAHTIGISHCPSFTDRLYPNQDSTMDKTFANNLKGSCPTADSNNTVNMDIRSPNVFDNKYYVDLMNRQGLFTSDQDLYTDRRTRGIVTSFAVNQSLFYEKFVIGMIKMGQMNVLTGGQGEIRNRCDRRNKAKKVDIATVVEELEETFSALF

SEQ ID NO: 46 - protein, 1144 aa, Solanum lycopersicum
MKRLCAFSFARRLFKPRTTHSQESKEKMEQSPIVNVEDNFDLQESKESMEKSPTVTANNNFDTQSATENEKENGSYAATNIILNHEFSDGLHSWHPNCCDAFVVPAGSGYHKGLAAKEGCCYAVVSNRKECWQGLEQDITSRVSAGSTYTVSACVGATGTFQGSVEVLATLKLVYQNSETSYLFVAKKAASEECWETLEGSFSLSTMPDQVIFYLEGPPAGTDLLIKSVVISCPSSTASDSSGTSSVYIDDDNIIINPQFDDGLNSWSGRGCKVALHDSMADGKITPMSGKSFASATERTQSWNGIQQDVTGRVKRKLAYEVSAVVRIYGNNVTTADLRSTLYVKAADNRERYIGIASVQATDKDWVKLQGKFLINDSPSQVVVFLEGPPAGTDILLNNLVIKHAAKAPPSSPPVIEDAGFGVNIITNTSLNDGTNGWFPLGNCTMSVQTGSPHIMPPMARDTLGAHEPLSGRYILVANRTQNWMGPAQMITEKVKLYLTYQVSAWVKIGQTSGPQNVNVALGVDSQWVNGGQAEISDDRWHEIGGSFRIEKQAAKIMVYIQGPVAGVDLMVAGLQIFPVDRRERFRHLKKQTAKLRKRDVMLKFSGSDSGNLFGTFVRVKQLQNSFPFGSAISRTNMDNEDFNAFFVKNFNWAVFGNELKWYWTEAQQGNLNYKDADELLDFCTKNNIQVRGHCIFWEVVGTVQAWVQSLNKNDLMTAVQNRLTGLLTRYKGKFPHYDVNNEMMHGSFYQDRLGKDIRVNMFKTAHQLDPSPILFVNDYHVEDGCDTRSYPEKYIEHILDLQEHGAPVGGIGIQGHIDSPVGPIVCSALDKLGTLGLPIWFTEVDVSSDNEYVRADDLEVMLREAYAHPAVEGIMLWGFWELFMSRTNAHLVDAEGDINEAGKRYLALKHEWSSHPHGHIDEQGQFSFSGFHGSYEVEVVTVSKKITKKFVVDKGDNALSSKENGNIILNHEFLDGLHLWRPNCCHAFVVPAGSGYRNGLTMAVVTKSTIWWQGLEQDITSRVSAGSSCTASDESKQEMEKAPPTITLILSSKEDEKDTGNIILNHDFSYGLYLWNPNCCEAFVVPSGYHKGLAAAIVTNRRECWHGLEQDITSKVSEGSTYTVSACVGASGTCPGSVELTCS

SEQ ID NO: 47 - protein, 216 aa, Solanum lycopersicum
MKRQFDAGETDWANFTGGDHVKKMKKEMLELEEERKLPMKRQFEANCAAKVSGDLKKMKQVIESPDADADADTEAVLLNENLLYEVLKHVDGRTLATAACVSKQWNQTALDERLWELICTRHSCNQQQQLRAVVLALGGFRRLYSLYLWPLSKPSPSPSASSSNPPASAWPCLPPAPTRPLKYAAANTRWGKDEVNLSLSLLSIRYYEKMNFKANK

SEQ ID NO: 48 - protein, 485 aa, Solanum lycopersicum
PSSFSIPQLKQSNMAIHLSALTISILVMVVSSAAVTSAAAEDMSIISYNEKHHTIGAGRTDDEVMSMYESWLVEHKKVYNALGEKDKRFQIFKDNLKYIDEHNAMPEKSYKLGLTKFADLTNEEYRSVYLGTKPDASRRLSSRQSDRYAPKVGDRLPESVDWVKKGVLVGVKDQGQCGSCWAFSAVAAIEAVNKIKTGDSISLSEQELVDCDTSSNNGCDGGLMDYAFEFVIKNGGLDTEEDYPYTGEDGRCDLTRKNAKVVTIDGYEDVPANDENAMKKAIASQPVSVAIEAGGKDFQHYKSGIFTGKCGAAVDHGVVAVGYGSENGMDYWIVRNSWGASWGEHGYLRMQRNIANPKGLCGIATVVSYPVKTGQNPPKPAPSPPSPVKPPTTCDDMYSCPSGTTCCCVYEYYHMCFAWGCCPMEGATCCKDHNSCCPHDYPVCNVKAGTCSISENNPLSVKAMSHILAKPIGSFSNQGMKNTIS

SEQ ID NO: 49 - protein, 338 aa, Solanum lycopersicum
MALPHHHLQLHIQQQPHQQQQQSKSYRDLYNNMDGQITNPVVYFNGSNLPEQSQHPPYIPPFQVVGLAPGTADDGGLDLQWNYGLEPKKKRPKEQDFMENNNSQISSVDLLQRRSVSTGLGLSLDNGRLASSCDSAFLGLVGDDIERELQRQDAEIDRYIKVQGDRLRQAVLEKVQANQIQAITYVEEKVLQKLRERDTEVDDINKKNMELELRMEQLALEANAWQQRAKYNENLINTLKVNLQHVYAQSRDSKEGCGDSEVDDTASCCNGRATDLHLLCRDSKEMKELMTCRVCRTNEVCMLLLPCKHLCLCKECESKLSLCPLCQSTKYIGMEVYM

SEQ ID NO: 50 - protein, 212 aa, Solanum lycopersicum
MKASFKARYEPDKAAANATVAFNAGDLKLRASMTDATVVKGPSLNGLALAVEKPGLFIVDYNVPKKDIRFQFMNSIKVLEKPLNLNYIHFHGDKRTILDGTLVVDSANKVSANHVMGSGSCKLKYTYVHGGITTFEPIYDTAKNAWDFMVSRKVYGDDVFKATYQTTSKNLGLEWSRSSKLNGSFKICASLCLLEERKIPKLSAETSWDFEM

SEQ ID NO: 51 - protein, 324 aa, Solanum lycopersicum
MLNAEHHFSLFKAKFGKIYASQEEHDHRLKVFKANLHRAKRHQLLDPSAEHGITQFSDLTPSEFRRTYLGLNKPRPNLNAEKAPILPTKDLPSDFDWREKGAVTDVKNQGSCGSCWSFSTTGAVEGAHFLATGELVSLSEQQLVDCDHECDPVEKNDCDAGCNGGLMTTAFEYTLKAGGLQLEKDYPYTGRNGKCHFDKSRIAASVSNFSVVGLDEDQIAANLLKHGPLAVGINAAWMQTYVRGVSCPLICFKRQDHGVLLVGYGSEGFAPIRLKNKPYWIIKNSWGKTWGEHGYYKICRGHNICGVDAMVSTVTATHTTNPNL

SEQ ID NO: 52 - protein, 431 aa, Solanum lycopersicum
MAGQTVIVSGLNPAAILQSTIGGAPVAAAAENGHTRKVVPLSKDALQDFMVSIITQKLQDDKQPFYVLDLGEVVSLMEQWNSALPNIRPFYAVKCNPEPSFLSMLSAMGSNFDCASRAEIEYVLSHGISPDRIVFANPCKPESDIIFAEKIGVNLTTYDSEDEVYKIRKHHPKCELLLRIKPMTDGNARCPMGPKYGALPEEIEPLLRTAQAARLTVSGVSFHIGSGDADSNAYLGAIAAAKQVFETAAQLGMPKMTVLDIGGGFTSGHQFTTAAPAVKSALETHFHDFPELTIIAEPGRFFAETAFTLATTIIGKRVRGELKEYWINDGLYGSMNCVLYDHATVTATPLACMSNRNNLNCGGSKTFPSTVFGPTCDALDTVLRDYQLPELQVNDWLIFPNMGAYTKAAGSNFNGFNTSAIVTHLAYAYPN

SEQ ID NO: 53 - protein, 439 aa, Solanum lycopersicum
MAKIAYLLTTLLVALVSLSFPSECKANYYYTSPPPPTPVYKYKSPPPPVYKYKSPPPPPPVYKSPPPPVYKYKSPPPPVYKYKSPPPPPPVYKYKSPPPPVYKYKSPPPPPPVYKYKSPPPPVYKYKSPPPPPPVYKYKSPPPPVYKYKSPPPPPPVYKYKSPPPPVYKYKSPPPPPPVYKYKSPPPPVYKYKSPPPPTPVYKYKSPPPPVYKYKSPPPPTPVYKYKSPPPPVYKYKSPPPPTPVYKYKSPPPPVYKYKSPPPPTPVYKYKSPPPPVYKYKSPPPPTPVYKYKSPPPPVYKYKSPPPPVYKYKSPPPPVYKYKSPPPPVYKYKSPPPPVYKYKSPPPPVYKYKSPPPPVYKYKSPPPPVYKYKSPPPPVYKYKSPPPPPPVYKYKSPPPPVYKYKSPPPPVYKYKSPPPPVHKSPAPYYYTSPPPPSHY

SEQ ID NO: 54 - protein, 216 aa, Solanum lycopersicum
MGSEGPKSVTIHVTGFKKFHGVAQNPTETAVSNLKYYVEKKGLPAGVTLGSCTVLETAGEGGLPTLLKVIEASSASNSSNNGQVIWLHLGVNSGATKFAIERLAVNEATFRCADELGWQPQRVPIVPEDGGINQIRKTCCPTESILELLKKKGFDVTLSDDAGRFVCNYVYYHSLRFAEEKGHKCLFVHVPTFHRVHQEKQMEFVAALLEAIASTC

SEQ ID NO: 55 - protein, 123 aa, Solanum lycopersicum
MDPNDLAKAFVEHYYSTFDSNRAALGNLYQEGSMLTFEGIKTQGSQNIVAKLTSLPFQQCHHTITTIDCQHSGPAGGMIVVVSGNLQLAGEQHALKFGQMFHLMPTPQGSFYVFNDIFRLNYG

SEQ ID NO: 56 - protein, 255 aa, Solanum lycopersicum
MAANQNVIVMRHGDRLDNFEELWVMKAERPWDPPLHQDGKIRAFCTGQKIRSSIDFPIHRVFVSPFLRCVQTAAEVVRALCDVADHNGESNVLSSNSDSVIIDPSKVKVSIEYGLCEMLNLVAVRASAAPKDGDFKFSISQYESELPAGTVDNTVEPVYKKLPEWEESLESARARYVKVVKALADKYPSENLLLVTHGEGVGSIFTELNKDSTVLEVAYCGHLHVKRSIQSGEKQPFTAGEFVYEKQTGIISAAK

SEQ ID NO: 57 - protein, 544 aa, Solanum lycopersicum
MESEKPQNPSPMEPQEEGSNKISKKEAAKLERQRRRQEAAAAAAATAVSTVSIEESDPLSGNYGDVELNDLQSKAVTGRKWTEVGSIAPEMKDRVVLVRGRVQTIRPVGKKIAFVVVRERGFTVQCVLTVKPELVSSQMVKYATSLSKESIIDVEGVVTIPEKPITGATQQQVEVQISKLYCVNRAVPTLPINIEDAARSEVEIEQALEKGEQLVRVNQDTRLNFRILDMRTPANQGIFRIQCQIENIFRQFLLSEGFVGIHTPKLIGGSSEGGSAVFRLDYKGQPACLAQSPQLHKQMAICGDFGRVFEVGPVFRAEDSFTHRHLCEFTGLDVEMEIKEHYSEVMDIVDRLFVAMFDSLNEKCKKELEAVGKQYPFEPLKYLRKTLRLTFAEGIQMLKEAGVEVDPLGDLNTESERKLGQLVAEKYGTEFYILHKYPQAVRPFYTMPCHDNPSYSNSFDVFIRGEEIISGAQRVHVPELLEKRAGECGIDVKTISTYIDSFRYGAPPHGGFGVGLERVVMLFCALNNIRKTSLFPRDPQRIVP

SEQ ID NO: 58 - protein, 804 aa, Solanum lycopersicum
MSGTPNKRPHEDGGNGGSSNHSYSSAPKYSHDDSGAFPKVMSSGTPEYHASFDVGQNARMPKIQRTESSRDADRRSPVLPMYRVSSCPVVSHPDHSVASENRLEPKEVNKDVKVENRDAKSEIRELYQGTKSDKDDRFENRADDGKDIKNSRDTYPEYKGDVKTDKDRFSGVSWKDPKEQTRGKRYPDLPVPVGNMDPWHASRTHGAAEIGKEVSNSENRDFAKVREAVAENKMDLKGDDKYKDKERKRKEGKHREWGERDKERNDCRNNLQLGNSTSDNKELLKEERESERWEKERNDLSKDKDRPKDWEKDHAKREVWNGVEREVLQSEKEVIDVPGKTNEPENSTVEQKKQKDHDNWKNTDRDGSERRKERDTDLEGERPEKRVRCHDKEPEEGDLDTEGGGEREREAFNYGVQQRKRMSRPRGSPMANRDPRFRSHTHENEGSQVKHDVSAVNYRVGECMPELIKLWKEYESSKADEASDSSPSDPTLEIRIPAEHVSATNRQVRGGQLWGTDIYTNDSDLVAVLMHTGYCRTTASPLLPTITELRATIRVLPPQNCYISTLRNNVRSRAWGAAVGCSYRIERCSVVKKGGGTIDLEPCLTHSSTLEPTLAPVAVERTMTTRAAASNALRQQRFVREVTIQFNLCNEPWLKYSISVVADKGLKKALFTSSRLKKGEVLYLETHSKRYELCFSGEKMVKATTSLMHEMDVDKPQSHNLHMANGEKNGVNGENTMVDMFRLSRCKKPLPQKLMQSVGIPLPLEHVEVLEENLEWENIQWSQTGVWIAGKEYPLTRAHFLSPN

SEQ ID NO: 59 - protein, 274 aa, Solanum lycopersicum
MSFDEDEESFEHTLLVVREVNVFKIPPRPTSGGYKCGEWLQTDKIWSGRLRVVSCKDRCEIRLEDPNSGELFAACFVPPGQRENSVESVLDSSRYFVLKIEDGTGKHAFIGLGFSERNEAFDFNVALSDHEKYVKRDIEKDGDEAETSSDGHIDIHPAVNHRLKEGETIRINVKNKSSSGMGMLSAAVQSKAITIAPPPSGANKIRSPLPPPPNDPAISRKTSTTASIALKGPNGSSMQSSDPISDLSQLERSLPSATGSGSSKKTTAAGWAAF

SEQ ID NO: 60 - protein, 289 aa, Solanum lycopersicum
MGDQIARGEDFESKAEKKLGGWAIFGSKYEDAADLFDKAGNCFKLAKSWDKAGAVYVKLANCHLKLDSKHEAANAYADAAHCYKKSNIKEAVSCLEQAVNLFLDIGRLNMSARYYKEIAELYEQEQNLDQAINYYEKAADLFQSEDVTTTANQCKQKVAQFSAQIEKYPRAIEIFEEIARHSVNNNLLKYGVRGHLLNAGICQLCKGDVVAINNALERYQELDPTFSGTRECKLLVDLAAAIDEEDVAKFTDAVKEYDSMTQLDAWRTTLLLRVKETLKAKELEEDDLT

SEQ ID NO: 61 - protein, 340 aa, Solanum lycopersicum
MEVIMNNHNNGTINTNTKIIHKNGSICNGNGNVNGNSHTHNNENKLVEFTNSIKPGWFSEFSALWPGEAFSLKIEKLLFQGKSDYQDVMLFESATYGKVLTLDGAIQHTENGGFPYTEMIVHLPLGSIPSPKKVLIIGGGIGFTLFEVSRYPSIEKIDIVEIDDVVVDVSRKFFPYLAAGFDDPRVSLIIGDGAAFVKAAQPGYYDAIIVDSSDPIGPAKDLFERPFFEAVAKALRPGGVVCTQAESIWLHMHLIKKIIANCRQVFKGSVNYAWTTVPTYPTGVIGYMLCSTEGPEVNFKNPVNSIDKYVKSKGPLKFYNSDIHKSAFILPSFARDLIES

SEQ ID NO: 62 - protein, 279 aa, Solanum lycopersicum
MGRSPCCEKAHTNKGAWTKEEDQRLINYIRAHGEGCWRSLPKAAGLSRCGKSCRLRWINYLRPDLKRGNFTEEEDELIIKLHSLLGNKWSVIAGRLPGRTDNEIKNYWNTHIKRKLISRGIDPQTHRPLNNNATNSHTTTNITTAVTTKNINLDFTNVDQKQPNIMIATSSSYDETKCNSGTTEETKPLEIIIPKIPSQVMINLELSIGLPLHTDHISSPESTASYNFLTTVAPPPTAAVPAAEMMAKTVCLCWQIGYHGGGGQWCGKCKNTNGFYRYC

SEQ ID NO: 63 - protein, 296 aa, Solanum lycopersicum
MATQDVRTLSTKEADIQMMLAAEVHLGTKNCDFQMERYAFKRRNDGIYIINLGKTWEKLQMAARVIVAIENPQDIIVQSARPYGQRAVLKFAQYTGAHAIAGRHTPGTFTNQLQTSYSEPRLLILTDPRTDHQPIKEAALGNIPTIAFCDTDSPMRYVDIGIPANNKGKHSIGVLFWILARMVLQMRGAINQGPKWDVMVDLFFYREPEEAKEQEEEVPAIADYADYSASAALGGDWTSSQIPEAQWTADAAAPAVGGGWAGDGAADGGWDAAAAPAPVPLPVPDVAPTSGATGWE

SEQ ID NO: 64 - protein, 367 aa, Solanum lycopersicum
MDSLNSYASSAAMAQQTWELENNIVTIDAPSGSKPENSASDAIFHYDDAAQTKFQREKPWASDPHYFKRVKISALALLKMVVHARSGGTIEVMGLMQGKTDGDAIIVMDAFALPVEGTETRVNAQADAYEYMVEYSQTNKQAGRLENVVGWYHSHPGYGCWLSGIDVSTQMLNQQYQEPFLAVVIDPTRTVSAGKVEIGAFRTYPEGYKPPDDPISEYQTIPLNKIEDFGVHCKQYYSLDITYFKSSLDCRLLDLLWNKYWVNTLSSSPLLGNGDYVAGQISDLAEKLEHAENQLSNSRYASLMAPPQRKKEEESQLAKITRDSAKITVEQVHGLMSQVIKDILFNSVGKSSKSQTESSGPEPMIES

SEQ ID NO: 65 - protein, 297 aa, Solanum lycopersicum
MASMIESAWTYLITNFNDFQLACLGSFFLHESVFFLSGLPFILLERAGWLSKYKIQTKNNTPAAQEKCITRLLLYHFCVNLPLMMASYPVFKLMGMRNTLPLPSWKVISTQVLFYFILEDFVFYWGHRILHTKWLYKHVHSVHHEYATPFGLTSEYAHPAEILFLGFATIVGPIITGPHLITLWLWMIVRVLETVEAHCGYHFPWSLSNFLPLYGGADFHDYHHRLLYTKSGNYSSTFVYMDWLFGTDKGYRRLKVLKSDACEAEGKEMRCERVSFGWSGIARRAYVQERIGEGFCC

SEQ ID NO: 66 - protein, 200 aa, Solanum lycopersicum
METTATPEEWPPDGQFPFDLFVSKKYKSFWDAGNIQFTDAVGNLFFSVDRRQPQNSAAQTHPQKLILDASDNTLIRLVRLTKGSWQGFMVSDNEEKALMFSVNKTLNTFTKLEFDIFLGDGHVEGAEADLKMKGSAFKRSCTIYKGNSIVAETSLMYTLGFRKHFIPRNRFRVTIFPGLTELSLVVALVVVFFDKQKFWL

SEQ ID NO: 67 - protein, 219 aa, Solanum lycopersicum
MGRRPARCYRQIKNKPYPKSRFCRGVPDPKIRIYDVGMKKKGVDEFPFCVHLVSWEKENVSSEALEAARIACNKYMTKSAGKDAFHLRVRVHPFHVLRINKMLSCAGADRLQTGMRGAFGKPQGVCARVAIGQVLLSVRCKDGNSNHAQEALRRAKFKFPGRQKIIVSRKWGFTKFSRTDYLRYKSENRILPDGVNAKLLGNHGRLAARQPGRAFLTSA

SEQ ID NO: 68 - protein, 437 aa, Solanum lycopersicum
MERYRILKELGDGTCGNVYKAINTETSEIVAVKKMKRKFYFWEECINLREIKSLRKLNHPNIIKLKEIVRENNELFFILEYMECNLYQLMKDRQRPFLEEEIRGLMSQVLQGLTHMHKNGYFHRDLKPENLLVTNDVIKIADFGLAREVSSSPPFTDYVSTRWYRAPEVLLQSSSYTPSIDMWAVGAVLAELFTLCPIFPGESEIDQLYKICCVLGPPDWTSFPELRSASRLFDINCFDITPANLSDFIPNASLEAIDLIKQLCSWDPLRRPTADQCLQHPFFHVDMSIPRPLEDPLQMNLSSVGPEPNLELNLWDFGTEKDDCYLGLTLAVNPTPSCLDLFPAKMASKSQGAGTDMFCSGFQDHSQHSVFWSLFPTDRHQISTPVDSSLSLSFSTIPQSTIGAPQSTSFGMTSLQSNFLERPFLAMSSSFQQGRCL

SEQ ID NO: 69 - protein, 476 aa, Solanum lycopersicum
MAAEAAVAVAAVAPVSAQVVANAFVQQYYHILHHSPGLVFRFYQDISKLGRPEEDGSMGITTTMQAINDKILSLNYGDLRAEIKSVDAQESFNGGVHVLVTGYLTGKDNLVRNFCQTFFLAPQDRGYFVLNDMFRYVETASQHDTVQVPVTDAVAPVTPEQIVADPPTAPQNHIPEQSTPPAEEADVGEVYNPSENGDVLIVEEEVPVSEVVHEVQDGSEVVAESDNKTGEVPKKSYASIVMHLKESAATFSPPPAPAARKPMTKSVVQVNQPAATTTDGPYSSSETIDNINDTEADGYSIYIKGLPMSATVALLADEFKKFGPIKNGGIQVRSNRQQGFCFGFVEFEVESAVQKAIEASPVLIGDRQAVVEEKRSTNSRLNARGGGRFQSGRGGGFRNDVGGRGRGNYLGGRGYGRDDFHGRNEFNNRGGNRGGSSNRGGEGYRRADNTGGRMSRGGGMSNGTAKGPAPRYSATA

SEQ ID NO: 70 - protein, 875 aa, Solanum lycopersicum
MQLSFSRPKSPMMFDLISISSHHPNSIINTINRPIQNPKIPSFFSSTTTHFSYSFNKYSNPLQFSVSSPTTSVLAHKGSEVEFEKGLEEEEAAAKTVVDAAMTITPAIRISDRKLMVKDRTILTNVPDNVLTTPGAASGPLEGVFLGAEFDQDNNRHVVPLGKLQDVRFLSCFRFKLWWMAQKMGDKGSEIPMETQFLLVETTDGSHLGSNDNKNDDNIVYAVFLPLIEGSFRAVLQGNAEDELELCLESGDKDTVGSAFNQAVYIHAGSDPFIVITEAIRAVKLHLKTFRQRHEKKLPKIVDYFGWCTWDAFYQEVTQEGVEAGLKSLTAGGIPPKFIIIDDGWQSVGGDPEVDKPLMRLTGLKENEKFQKKEDPTLGIKNIVNIAKEKYGLNYVYVWHAITGYWGGVRPGVKGMEEYGSVVKYPDITKGVMENEPGWKTDAIAVQGLGLVNPKSAYKFYNEMHSYLASAGVDGLKVDVQCILETLGGGLGGRVELTKQYHQALDASVARNFPDNGCIACMSHNTDALYCSKQTAVVRASDDFYPRDPASHTIHIACVAYNSVFLGEIMLPDWDMFHSLHPAAEYHGSARALSGGPVYVSDAPGKHNFDVLRKLVLPDGSILRARLPGRPTKDSLFTDPSRDGVSLLKIWNMNKYTGVLGIYNCQGAAWSTVERKTTFHKTNSEAITGYIRGCDVHFISEAALDPNWSGDTVLYSHGSAELVVLPYNAAMPVSFKILEHETYTVTPIKVLAPGFSFAPLGLIDMYNAGGAIEGLKYEVKAGAELSELEAGYQGEGNLVAEDKIENLSTEAVAVVSMEVRGCGRFGVYSSVKPRKCSVGGDMVDFAYNSESGLLTLNLDAMPPADQKVHIIEVEV

SEQ ID NO: 71 - protein, 524 aa, Solanum lycopersicum
MASTCLDVSFLGNFCNNWSFRPCTRLTPCISQVRNSLLLGKNKNYTSSVGYKGIPLTEISCVPSAVASSSELPPLVSALKASAEENAASFHFPGHNRGQAAPSSLTQLIGAKPFLHDMPELPELDNLFSPEGPILEAQKEAARLFGALETWFLVGGSTCGVHAAIMATCSPGDTLILPRNSHISAISAMVLSGALPKYIVPEYDFKWDVAGGITPSQVNDAIKELEMEGRKAAALLVVSPTYHELPSSSLSQGADLAVQSTHKVLCSLTQSSMLHMQGNLVNRERISKSLQMLQSSSPSYLLLASLDAARAQLSENREAVFDKAMDLALEARSLISKIPGISIIEFPSFSSFFQIDPLRLTIGVWQLGLSGFEADDILCNDFGVVCELVGTKSFTLAFNLGTQRDHVLRLVAGLNHLSQTSHFPQPVKEEGKNVNHFVCLDDIRIRMSPREAFFATKRKVSIRDSLGEICGELVCPYPPGIPVLIPGEIITAEALNYLLEIRSKGAVITGAADFSLSSFVVCVT

SEQ ID NO: 72 - protein, 589 aa, Solanum lycopersicum
MRRSPNMFPKSFFTLLFIFILSTAVYGGRVIVDQSSSDLISDGVHGGGGRNSTSIGRERGIISLFAAPVEEACEQSYGFLPCTKTALGNVFLIVVYGYLMFTAATYLSSGSELLLEILGPGLIGGLFLPILGALPDAMLILVSGITGSAAEAQSQVSVGIGLLAGSTVMLLTAIWGTCCTVGKCDIVNSIAVDLKDTKGLSFTGSGVSTDIWTSYAAMIMAVSVLPFIVVQLPQIFHSNSGRHLSVLIALIISLSLLLSYCLYQVFQPWIQTRKLDYVKHKHVISGILKHLKKHALGRLFTDHGTPNIEVLEKLFNAIDENGDKHLSHSELRALVVGIRLEEINLDENDAIAKVMKDFDTSLDEQVDLHEFITGVEKWLYEAKSTNGSSAQAGPNTINYIDDFHEETRREHSLLGEDQSDEIVEGVENPRKVAIKAASLLLLGTIIAAVFADPLVDAVNNFSSATSIPSFFISFIALPLATNSSEAVSAIIFATRKKQRSASLTFSELYGAVTMNNLLCLSVFLAIVYARGLTWNFSSEVLVILIVCIVIGVLGSIRTTFPLWTSLIAFAFYPFSLVLVYVLDYVFGWS

SEQ ID NO: 73 - protein, 457 aa, Solanum lycopersicum
MQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPSDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGF

SEQ ID NO: 74 - protein, 383 aa, Solanum lycopersicum
MGWLRYVESGRKIDCISNPNTAYPIPVLVEEVDDIISVAQYVIVVEKESVFQRLGRGYPDVPTRRFLRLLIDKLHLPVYGLVDCDPYGFDILTTYKFGSLQMAYDATFLRVSEIQWLGVFVQDCDNYSIPQQCLLPLTAEDKRKIEAMLPRCYLQREVPQWRFELELLLHKGVKFEIEALSVHSLTFLSHEYLPSKIHNGGYIVDVHCDACKMKMVDVLSSICGVYSVTIDSEDGTAKISGEVDPNLLLRALSRSGLGNHAEVKWVRLKHPMLSNSHMTDGYGSYNHGYGSYNHGYGSHNHGYGSYNPGYGYNSYGHGHGHGHGYSSIGGPFMQRRSLPEYNYGYEGHHNYQYPFRGTAQDSYATNYYSGALPSPRPYNSYYM

SEQ ID NO: 75 - protein, 699 aa, Solanum lycopersicum
MSDVETFAFQAEINQLLSLIINTFYSNKEIFLRELISNSSDALDKIRFESLTDKSKLDGQPELFIHIIPDKANNTLTIIDSGIGMTKADLVNNLGTIARSGTKEFMEALAAGADVSMIGQFGVGFYSAYLVAEKVVVTTKHNDDEQYVWESQAGGSFTVTRDTSGENLGRGTKMVLYLKEDQLEYLEERRLKDLIKKHSEFISYPISLWVEKTIEKEISDDEEEEEKKDEEGKVEEVDEEKEKEEKKKKKVKEVSNEWSLVNKQKPIWMRKPEEITKEEYAAFYKSLTNDWEEHLAVKHFSVEGQLEFKAVLFVPKRAPFDLFDTKKKPNNIKLYVRRVFIMDNCDELIPEYLSFVKGIVDSEDLPLNISRETLQQNKILKVIRKNLVKKCVELFFEIAENKEDYNKFYEAFSKNLKLGIHEDSQNRAKFAELLRYHSTKSGDEMTSLKDYVTRMKEGQNDIYYITGESKKAVENSPFLEKLKKKGYEVLYMVDAIDEYSIGQLKEFEGKKLVSATKEGLKLDESEDEKKKQEELKEKFEGLCKVMKDVLGDKVEKVIVSDRVVDSPCCLVTGEYGWTANMERIMKAQALRDSSMAGYMSSKKTMEINPENSIMDELRKRADADKNDKSVKDLVLLLFETALLTSGFSLEEPNTFGNRIHRMLKLGLSIDEESGDADADMPALEDPEADAEGSKMEEVD

SEQ ID NO: 76 - protein, 537 aa, Solanum lycopersicum
PFELGDNSYKHNKYIIQTINFSIISSLFTMATPITKPPSPACTKIIEEITRIYKSLPPRPSILEIEASISVVKSVETEEKIELDEISKRRVEIEESVPAELYSVLEKVRKAMVLFQSKEQKKEAVELIELDKTYQDFDELIQEATELVCEDTQMGKINSFEDPMVEIGKKDEVLKKGLVTSGELINVSSSGIKHKEKYSLMKVAALIENAAKTRARVVDLQNKLMDKIEWLPLSLGKLVNVTELNVADNQIMALPTTIGSLNGLTKLDLHSNQIINLPDSFGELINLTDLDLHANRLKSLPASFRNLVNLIDLDLGSNRFTHLPDFVGNLTSLKRLNVETNQLEELPYTIGFCSSLVELRLDFNQLKALPEAMGMLEHLEILTLHINRIKGLPTTMGNLSRLRELDVSFNEVENIPETFCFAVSLEKLNLANNFADLKTLPRSIGNLENLEELDISNSQIRTLPDSFRLLSKLKTFRADETPLEVPPRQIIKLGAQVVVEYMAEFVTKNELQLQRPKRRPFFCPSCLFPKTEGKRRI

SEQ ID NO: 77 - protein, 291 aa, Solanum lycopersicum
MEKIRKASHAGSWYTDNPLELEEQLDGWLRAAGLVKSSDVRGVIAPHAGYSYSGRAAAYAFGNIDPTNISRIFLLGPSHHYYTPKCALSRATVYKTPIGDLPIDEEVNDELKATGHFEYMDLRVDEAEHSMEMHLPYLAKVFQGYPVKIVPILVGALSAESEALYGRFLAKYVDDSKNFFSVSSDFCHWGSRFNYIHYDKSHGAIYKSIEVLDKMGMDIIETGDPDAFKLYLSETDNTICGRHPISVFLHMLKNSSTKIKIRFLRYEQSSQCKSMRDSSVSYASAVGKVDG

SEQ ID NO: 78 - protein, 1036 aa, Solanum lycopersicum
MERARKLANRAILKRLVSQSKQSRSNEIPSPSSLYRPSRYVSSLSPYTFQARNSVKSFNTQQVRSISVEALKPSDTFPRRHNSATPEEQTKMAEFCGFQSLDALIDATVPQSIRSESMKLPKFDGGLTESQMIEHMQKLASKNKVFKSYIGMGYYNTYVPPVILRNLLENPAWYTQYTPYQAEISQGRLESLLNYQTMITDLTGLPMSNASLLDEGTAAAEAMAMCNNILKGKKKTFLIANNCHPQTIEICKTRADGFDLKVVTVDLKDIDYKSGDVCGVLVQYPGTEGEILDYGEFIKNAHAHGVKVVMASDLLALTMLKPPGELGADIVVGSAQRFGVPMGYGGPHAAFLATSQEYKRMMPGRIIGLSVDSTGKPALRMAMQTREQHIRRDKATSNICTAQALLANMAAMYAVYHGPEGLKTIGQRVHGLAGTFSAGLKKLGTVEVQDLPFFDTVKVKCSDAKAIADVATKNDINVRIVDNNTITVSFDETTTLEDVDDLFKVFALGKPVPFTAQSIAQEVENLIPSGLTRETPFLTHQIFNSYHTEHELLRYLHKLQSKDLSLCHSMIPLGSCTMKLNATTEMMPVTWPSFANIHPFAPTEQAAGYQEMFDDLGALLCTITGFDSFSLQPNAGAAGEYAGLMVIRAYHMSRGDHHRNVCIIPVSAHGTNPASAAMCGMKIVAVGTDAKGNINIEELRKAAEAHKDNLSALMVTYPSTHGVYEEGIDEICKIIHDNGGQVYMDGANMNAQVGLTSPGFIGADVCHLNLHKTFCIPHGGGGPGMGPIGVKKHLAPYLPSHPVVSTGGIPSPDQSKPLGAISAAPWGSALILPISYTYIAMMGSKGLTDASKIAILSANYMAKRLEKHYPVLFRGVNGTCAHEFIIDLRGFKNTAGIEPEDVAKRLIDYGFHGPTMSWPVPGTLMIEPTESESKAELDRFCDALISIREEIAQIEKGNVDINNNVLKGAPHPPSMLMADAWTKPYSREYAAYPAPWLRSAKFWPTTGRVDNVYGDRNLICTLLPVSEMAEEKAATA

SEQ ID NO: 79 - protein, 151 aa, Solanum lycopersicum
MHVEPHRRALSVERWVCLGRAIGRLGPHFLFQQNTKSSSFFFTALLFSARVSGISRCVLKMSRVYVGNLDPRVSERELEDEFRIFGVIRSVWVARRPLATLLLTSMIGGMHKMQSKSWMVRMDGEWSFHIILEVEVVGAVEEVEVDLEALI

SEQ ID NO: 80 - protein, 430 aa, Solanum lycopersicum
MDYGCLKKIATKSPTTTIFLPYLLRSVLTTRFQLLVAGSFCNSSGDHRNSEVFTSFSGSKEMEFFFRTQTEETSDCSFNGQDIQRCPFLSNINKPTNFSFFSALNFPSPVKGGKGPIFEDGPNFDMAFKVFHGKDGVVPLSGRSQFFNDNIEVESAPQFNPLAAKAATISLSSFGAGGPFSFDSFSRKWNSQKKKPESSKKKKPSSQDKSSKHEAMGNEWLESGNCPIAKSYRAVSGVLPLVASAFQLPPGMKLKCPPAVVAARAALARTAFVKTVRPQPLSSKMLVIGALGMAANIPLGIWREHTEKFSLSWFTAVHAAVPFIAMLRKSVVMPKTAMALTIAASILGQVIGSRAERLRMKAKAESVKLVAQTGSDGVIAQSSTEGVIAGLNSIQVSGMPGVHCGTQGMLKDQPSKESANTISPSASLCF

SEQ ID NO: 81 - protein, 302 aa, Solanum lycopersicum
MKTVLFRTGSGSIPAVVPGSQRAVSVSTRESIGKVFNGEKKKGCSSPMISLYSEVNRRGIRRVSSDSDVIRSGFEGSSVTRKLSKIGSRSLSSIIPEEDCGSEQNELEVLRSIGSLSLTEDRRSYAGDWHRNSIPVDELGFSGGGIGSTKNKKFGGNGDGRDEIDADSLTGGKSDPRKIGAYYKEMLNSDPMNPLLLRNYGKYLHEVERDFVKAEECYGRAILASPGDGEVLSLYGKLVWETQRDENRAKSYFDQAVHASPNDCTVLGSYAHFMWEAEGDQDEDEDEERETVAAGASMVAAY

SEQ ID NO: 82 - protein, 485 aa, Solanum lycopersicum
MSSITYSNLILKRRESRINGGSHLQYNGLRAIETVKLTANSASCKPKAVKCGRIRAMASPAVSAPKRETDPKKRVVITGMGLVSVFGSDIDNFYNKLLEGQSGISLIDSFDASSYPVRFAGQIRDFSSEGYIDGKNDRRLDNCWRYCLVAGKRALDDANLGQQVLETMDKTRIGVLVGSGMGGLNVFSDGVEALLQRGYKKVSPFFIPYSITNMGSALLAIETGLMGPNYSISTACASANHSFCAAANHIRRGDADIMVTGGTEAGVTATGVGGFIACRALSQRNDDYEKASRPWDTNRDGFVVGEGSGILVMESLEHALKRGASIIAEYLGGAATCDAHHMTDPRPDGLGVWSCITKGLLDARISPEEVNYINAHATSTLAGDLAEVNAIKKVFKDTSEIKMNGTKSMIGHGLGAAGGIEAIVTIKAITTGWLHPTINQHELEPQVTIDTVPNVKKQHEVNVGISNSFGFGGHNSVVVFAPYRP

SEQ ID NO: 83 - protein, 390 aa, Solanum lycopersicum
METFLFTSESVNEGHPDKLCDQVSDAILDACLEQDPESKVACETCTKTNMVMVFGEITTKATVDYEKIVRDTCRGIGFVSADVGLDADNCKVLVNIEQQSPDIAQGVHGHLTKKPEEIGAGDQGHMFGYATDETPELMPLTHVLATKLGAKLTEVRKNKTCPWLRPDGKTQVTVEYKNDNGAMVPIRVHTVLISTQHDETVTNDQIAQDLKEHVIKPVIPAKYLDENTIFHLNPSGRFVIGGPHGDAGLTGRKIIIDTYGGWGAHGGGAFSGKDPTKVDRSGAYIVRQAAKSVVASGLARRCIVQVSYAIGVAEPLSVFVDTYKTGTIPDKDILVLIKENFDFRPGMMSINLDLLRGGNYRYQKTAAYGHFGRDDPDFTWETVKVLKPKA

SEQ ID NO: 84 - protein, 444 aa, Solanum lycopersicum
MATIKSIKARQIFDSRGNPTVEVDVHISNGVFARAAVPSGASTGIYEALELRDGGSDYLGKGVSKAVNNVNSIIGPALVGKDPTDQTGLDNFMVHQLDGTQNEWGWCKEKLGANAILAVSLAVCKAGAAVRNVPLYKHIADLAGNKKLVLPVPAFNVINGGSHAGNKLAMQEFMILPVGAANFKEAMKMGCEVYHHLKAVIKKKYGQDATNVGDEGGFAPNIQENKEGLELLKTAIEKAGYTGKVVIGMDVAASEFYGKDKSYDLNFKEESNDGSQKISGDQLKDLYKSFVSEYPIVSIEDPFDQDDWETYAKLTAEIGEQVQIVGDDLLVTNPKRVAKAIAEKTCNALLLKVNQIGSVTESIEAVKMSKKAGWGVMTSHRSGETEDTFIADLAVGLSTGQIKTGAPCRSERLAKYNQLLRIEEELGSEAVYAGASFRKPVEPY

SEQ ID NO: 85 - protein, 109 aa, Solanum lycopersicum
MDAQSKSDSFSSSNLRIIVQNNPSESQLSELGIKSWPKWACSPGKYQLKFDAEETCYLLRGKVKVYPKNTIEMSPVEFGAGDLVIIPKGLCCTWDVTLPVDKHYKFHSS

SEQ ID NO: 86 - protein, 638 aa, Solanum lycopersicum
MFPLLHLFIFLCLSFSYCCYGQIINSSSSSCPLDFTSLRKIIDQNSKRPTLNSTAQCQYIRQGLRLVQSEYLRRTNSFFPPMSSADSCWNSYQSLVNDYVQNFDIRSSCGFQTAWISQGCMNITTRFEFESKVSPAAITNIVSSCNQSLENNSPCATCTTSLSGLASFLPGPSVGNLFDCAAYPSVYAAAFANQFGPTDKGTAKCLFLLDVSVGSSGKGKKSVVVIVVVVVCVVVFAVVVFGYWFMWRKRKIGLAKKWNTKRLESNLSSRLDSISGSTTLIRYSFDEIKAATKNFSRLNIVGTGGYGNVYKGVLPGGIEVALKRFKNCSVAGDANFTHEVEVIASVRHVNLVALRGYCTATTPFEGHQRIIVCDLMKNGSLHDHLFGTRHEKLSWGIRQKVAIGTARGLAYLHYGAQPGIIHRDIKASNILLDESFEPKVADFGLAKFTPEGMTHLSTRVAGTMGYVAPEYALYGQLTERSDVYSFGVVLLELLSGKKAIMEFKDGQPTLVTDWAWSLVREGRALDVLEDSIPHLGPPEVMEKYVLVAVLCSHPELYARPTMDQVVNMLDAEIPVPTIPERPISLIADLDDIERSVSSGGNSGNLSTAAGYQPYIFERETPLADSDSVRARTLDQRSR

SEQ ID NO: 87 - protein, 305 aa, Solanum lycopersicum
MRNMLFASLYPARLHFLKTLIFKISSSSTLSALDLPSRSSRTLYVGNLPGDVREREVEDLFYKYGPIAHIELKIPPRPPGYAFVEFEEARDAEDAIRGRDGYDFDGHRLRVELAHGGRGNSSSNDRYGGGGGGRGQRGGGVSRRSDYRVLVTGLPHSASWQDLKDHMRRAGDVCFSQVFRDGSGTTGIIDYTNYDDMKYAIKKLDDSEFRNAFSRATIRVKEYDRSRSRSRSRSRSYSRGKSVSRSRSRSRSRSRSKSKSKSPKVKSSKRSRSRSRSVSSQSRSGSKGRPVSRFWICAAVCGFAY

SEQ ID NO: 88 - protein, 485 aa, Solanum lycopersicum
MALLVEKTTSGREYKVKDMSQADFGRLEIELAEVEMPGLMASRAEFGPSQPFKGAKITGSLHMTIQTAVLIETLTALGAEVRWCSCNIFSTQDHAAAAIARDSAAVFAWKGETLQEYWWCTERALDWGPGGGPDLIVDDGGDATLLIHEGVKAEEEFAKNGTVPDPTSTDNVEFQLVLTIIKESLKTDPLRYTKMKERLVGVSEETTTGVKRLYQMQANGSLLFPAINVNDSVTKSKFDNLYGCRHSLPDGLMRATDVMIAGKVALVAGYGDVGKGCAAAMKQAGARVIVTEIDPICALQATMEGLQVLPLEDVVSEVDIFVTTTGNKDIIMVDHMRKMKNNAIVCNIGHFDNEIDMHGLETFPGVKRITIKPQTDRWVFPDTNSGIIVLAEGRLMNLGCATGHPSFVMSCSFTNQVIAQLELWNERSSGKYEKKVYVLPKHLDEKVAALHLGKLGAKLTKLTKDQADYISVPVEGPYKPAHYRY

SEQ ID NO: 89 - protein, 346 aa, Solanum lycopersicum
MTKEGKNVKRIKLGSEGLEVSVQGLGCLGMSDFYGPPKPEPDMIELIHHAINSGITFFDTSNSYGPYTNEILLGKALKGGMRERVELATKFGIHFADGKIEVRGEPEYVRAACEASLKRLDVDCIDLYYQHRIDTRVPIEVTVGELKKLVEEGKIKYIGLSEASASTIRRAHAVHPITAVQLEWSLWSRDVEEEIIPTCRELGIGIVAYSPLGRGFLSSGPKLVEDLTNEDFRKHLPRFQGENLEHNKKLYERICELATKKGCTPSQLALAWVHHQGNDVCPIPGTTKIENFNQNIGALSIKLTSEDMAELESIASANAVQGDRYGSAAATYKDSETPPISAWKAT

SEQ ID NO: 90 - protein, 405 aa, Solanum lycopersicum
MKNGMAECAVCHSKLVSPSVKTISRAYDRHRSKISSKQRALNVLLVVGDCMLVGLQPVLVFMSKVDGKFKFSPVSVNFLTEATKVVFAIIMLLIQARHQKVGEKPLLSISTFVQAARNNVLLAVPALLYAINNYLKFIMQLYFNPATVKMLSNLKVLVIAVLLKFIMKRRFSVIQWEALALLLIGISINQLRSLPEGTTSLALPVTTIAYIYTLIFVTVPSMASVFNEYALKSQYDTSIYLQNLFLYGYGAIFNFLAILGIAVFKGPGSLDIFQGHSKATMLLIVNNAAQGILSSFFFKYADTILKKYSSTVATIFTGIASALLFGHTLTVNFLLGISVVFISMHQFFSPLSKVKDDQQNGTVELIDVRENHSPKDSSFVNMAAGANEEASHRVGPDERQPLLPR

SEQ ID NO: 91 - protein, 419 aa, Solanum lycopersicum
MAFSSLLKPAASFVRPSHRSQVSCAGLHHSSNSVKLQSSIFGDAVTILQSSSLQKSGACSIQPIRATATELPPTVPRSQTGGKTRVGINGFGRIGRLVLRIATFRDDIEVVAVNDPFIDAKYMAYMLKYDSTHGVYSGSISVLDDSTLEINGKQIKVSSKRDPADIPWGDLGADYVVESSGVFTTIDKASAHKKGGAKKVVISAPSADAPMFVVGVNEKTYKANMDVVSNASCTTNCLAPLAKVVHEEFGIVEGLMTTVHATTATQKTVDGPSMKDWRGGRGAGQNIIPSSTGAAKAVGKVLPELNGKLTGMAFRVPTPNVSVVDLTCRLDKSASYDDVKAAIKYASEGPLKGILGYTDEDVVSNDFVGDSRSSIFDAKAGIGLSKSFVKLVSWYDNEWGYSNRVLDLIEHMALVASIH

SEQ ID NO: 92 - protein, 526 aa, Solanum lycopersicum
MAGKQLDVPRTSGGNLKEQLVRRTLQNVRSQGHIYVELREDGKRLIFFCTLCHSPCYSDSVLFNHLKGNLHTEMLAAAKATLLKPNPWPFNDGVLFFNDPEQDKQDKQSPNVNVGKSRLVDTCLEDESSVAIVEYDDNLRHNEDTYVSEYEYGLLDSELIGNEESDYLVIPGVLCKDELSDLEVKHIGIGKIAARISVRGIDSKSIRRIWCEWLAKKDSDDMDTSVVPDHDFAVVTFPYNYNLGRSPLLDDRFLLPSSPYSESEETSVTGKRKRKSFSDPEDFSESLSNHCDSSGEESQSTNNSNMKLILGTCDDQLVSSRIISSKTMRRELRKQQRVASERMCDICQQKMLPGKDVATLLSWKSGKLMCSSRNMSGAFHLFHVSCLIHWILQCELQTSVKPVDEPKMEPKAKRRSKKKTGTKHNAKEKEDETKSARRINSVFCPECQGTGICIEGDELEKPPVSLSEVYRLKIKLSDARKAWMKNPEVLQNCSTGFDLPPEHDDLLQEYVSPLKLLHFYRANVSH

SEQ ID NO: 93 - protein, 674 aa, Solanum lycopersicum
MESRHSIHHVLLLVLFGLSALCQCSATGVEANQTAVLLVNASEASARRIPDTLFGIFFEEINHAGAGGLWAELVNNRGFEGGGPNVPSNIDPWSIIGDESKVIVSTDRSSCFDRNKIAVQVQVLCDHTGANICPDGGVGIYNPGFWGMNIEQGKSYKLVLYVRSEESVNVSVALTGSNGLQKLAAANIVAADVSSWTKVEILLEATGTDPNSRLELRSSKKGVIWFDQVSLMPTDTYKGHGFRKDLFGMLKDLKPAFIRFPGGCFVEGDWLRNAFRWKETIGQWEERPGHFGDVWNYWTDDGLGHFEFLQLAEDLDSLPVWVFNNGVSHHDQVDTSSILPFVQEILDGLEFARGDPTSTWGSIRAKMGHPEPFDLRYVAIGNEDCGKTQYRGNYLKFYTAIKDKYPDIKIISNCDGSTRPLDHPADLYDFHIYSSASSVFSNARHFDSAPRRGPKAFVSEYAVTGNDAGKGSLLAALGEAGFLIGVEKNSEAIEMASYAPLFVNDNDRRWNPDAIVFTSSQMYGTPSYWMQHFFKESNGATLLSSSLQANPSNSLIASAITWRNSLDNNDYLRIKVVNFGTTAVITKISLTGLGQNSLETLFGAVMTELTSNNVMDENSFREPNKVIPVKTQVEKVSDNMDVVLAPRSLNSIDFLLRKSINNNVDTASVLKSSC

SEQ ID NO: 94 - protein, 437 aa, Solanum lycopersicum
MADGQENDKNIEIWKMKKLIKALESARGNGTSMISLIIPPGDQISRITKMLAEEYGTASNIKSRVNRQSVLGAITSAQQRLKLYNKVPPNGLVLYTGTIMTEDGKEKKVTFDLTPFKPINASLYLCDNKFHTGPLGELLESDEKFGFIVMDGNGTLFGTLSGNTREILHKFTVDLPKKHGRGGQSALRFARLRMEKRHNYVRKTAELATQFFINPATSQPNVSGLILAGSADFKTELSQSDMFDPRLQAKILNVVDVSYGGENGFSQAIELSAEILSNVKFIQEKRLIGKFFEEISQDTGKYVFGVDDTIKALEMGAVETLVVWENLDINRYVLKNSVTNEIVIKHLNKDQEADQSNFKDSENSAELEVQDKMPLLEWFANEYKTFGCSLEFVTNRSQEGSQFCRGFGGIGGILRYQLDMRSFDEPSDEGEYFEDSD

SEQ ID NO: 95 - protein, 378 aa, Solanum lycopersicum
MAKNSFLLHILLFFVLILSLCDGILSQYELEELTWLDDKDDEISMFHSQHSSMRRCDFTSGKWVFDQSYPLYDSTCPYLSTAVTCTKNGRPDSDYEKWRWKPHGCEIPRFNALEFLGRMRKKRIMLVGDSIMRNQWESLVCLVQSVIPMARKTVTYVGPTMAFHAMDFETTIEFCWAPFLVELKKGPENKRILHLDMIEENAKYWRGADVLVFDSAHWWTHSDKYSSWDLIMEGNSFYRNMNPMVAYEKGLMTWAKWVDLNLDPRKTRVFFRSMSPRHNRENGWKCFNQREPLEFFSHPHVPEPLLVLKEVLRGMSFPVSFQDITTMTALRRDGHPSVYSKFVSQTGKQHLGDYKSDCSHWCLPGVPDTWNEMLNVML

SEQ ID NO: 96 - protein, 354 aa, Solanum lycopersicum
MSLLSDLINLNLSGDTQKIIAEYIWIGGSGMDMRSKARTLPGPVTSPAELPKWNYDGSSTGQAPGEDSEVILYPQAIFKDPFRRGNNILVMCDAYTPAGEPIPTNKRHAAAKVFSHPDVAAEETWYGIEQEYTLLQREVNWPLGWPIGGFPGPQGPYYCGTGADKAFGRDIVDAHYKACLYAGINISGINGEVMPGQWEFQVGPSVGISAGDEVWVARYILERIAEIAGVVVSFDPKPIPGDWNGAGAHTNYSTKSMREDGGYEIILKAIEKLGLKHKEHIAAYGEGNERRLTGKHETANINTFKWGVANRGASVRVGRDTEKAGKGYFEDRRPASNMDPYVVTSMIAETTIIG

SEQ ID NO: 97 - protein, 166 aa, Solanum lycopersicum
MIQENIVILLSLLLIVPISWAINNPLIELSSREELAKIAGYGEEKLSTVFLHGTLLCGHTPHPVPGASVAVYCGTSGPGRVRKSSWAKNVTNESGDFIIDVPSHLHANIHDPNLCHIQILHLPKDYSLCHHHSTKRKTFTLTSSNIGVRVYTTPTIYLTPKASSRV

SEQ ID NO: 98 - protein, 215 aa, Solanum lycopersicum
MLAHCTKSSLPAADAHLPTLRLPNPRRSPAPELHFGFNSFSTRTSKLQVKALDRNSNNSSTGDGVGASKNAASSPSSSVPNTNYVVPLDKASSCITRPLAEILRDLNKRIPDNIIKAGNNDDHSTFITWYHANRMLSFYAPGWCGEVRDVIFSDNGSVTVVYRVTIRGSDGEAHRESTGTVSPGNGQYTDVVAAAEEIAFCRACARFGLGLYLYH

SEQ ID NO: 99 - protein, 252 aa, Solanum lycopersicum
MASSSAKILLPLSLLFTLLSLSQSTNPNFILTLVNNCPYTIWPAIQPNAGHPVLERGGFTLHSLTHRSFPAPNAHWSGRIWARTGCNYQHGKFYCATGDCGGRIECDGLGGAAPATLAQFVLHHGHADFSTYGVSLVDGFNIPLTVTPHEGKGVCPVVGCRANLLESCPAVLQFRSHGGHGPVVGCKSACEAFKSDEFCCRNHYNSPQTCKPSSYSQFFKHACPATFTYAHDSPSLMHECSSPRELKVIFCH

SEQ ID NO: 100 - protein, 494 aa, Solanum lycopersicum
MIGWFKQLHTAFGFSFLWLVCLIYFTQGFRSFVWTAVSYQLKDNLKLSPSASQFVTSIAFFPWSIKPLYGILSDCFPIRGRKRVPYLMLATLLSLLPWLILGLSDFLRNTRTELMILLTLQNLGSAMADVVIDAMIAEAVRFERASFAGDLQSVSWLSMAFGGICGSLLGGYALDNIRIDMIFLLFSVLPALQLFSCGLVEEGSVQGKALPEGSALNGSDMTSGIVDEENKFSGEKSKVSTLRRKKSQNNTKRVPTTESKFQAPDKKGSSLSPWYQSLKMATSTLFRAFRQPAILRPMAWFFLAHVTVPNLSTVMFYYQTEFLKLEASFLGTARVLGWLSLMIGTLTYNRYLKRMRLRHILMFTQVCLAILTILDMVLVSRANVSLGISDKVTVLFGSALSDGINQFKFMPFLILSGQLCPPGVEGTLFALFMSINNLGSTVGSFVGAGLASVLNISSGSFDNLFLGIGIQVLCTFIPVAFLFLIPKEATGISA

SEQ ID NO: 101 - protein, 666 aa, Solanum lycopersicum
MAQIQVQHQTPVAGGNGVAVAAAAAGAAAAVAAPGGGATSQFTSTSLYVGDLDFNITDSQLYDLFNHVGQVVSVRVCRDLSTRRSLGYGYVNYSNPSDAARAMELLNFTPVNGKSIRVMYSHRDPTLRKSGSANIFIKNLDKSIDNKALHDTFSSFGNILSCKIATDSNGQSKGYGFVQFDNEESAQGAIDKLNGMLMNDKQVYVGHFLRKQERESTTGMTKFNNVYVKNLAESATDDELKNVFGEFGTITSAVVMRDADGKSKGFGFVNFENADDAAKAVEALNGKKFDEKEWYVGKAQKKSEREQELKNKFEQTAKEAVDKYQGVNLYVKNLDDTIDDEKLKELFAEFGTITSCKVMRDPSGISRGSGFVAFSTPEEASRALSEMNGKMIVSKPLYVALAQRKEERRAKLQAQFSQLRPVAMPPSLAPRMPIYPPGAPGIGQQLFYGQGPPALIPPQAGFGYQQQLVPGMRPGGAPMPNFFMPMVQQGQQGQRPGGRRGAGPVQQSQQPMPLMQQQMMPRGRMYRYPPGRNVPDGPMPGVAGGMLSVPYDMGGMLPRDAAAMGQPVPISTLATALANAPPEQQRTMLGESLYPLVDQLEHEHAAKVTGMLLEMDQTEVLHLLESPEALKAKVSEAMDVLRNVQQATSSPADQLASLSLNDNLVS

SEQ ID NO: 102 - protein, 485 aa, Solanum lycopersicum
MGTESNYQMKVVKGDHGYVLEDVPHLTDYIPDLPTHDNPLRSNPAYSVVKQYFVDMDDTVPQKVVVHKDSPRGVHFRRAGPRQKVYFNSDDVRACIVTCGGLCPGLNTVIREIVHSLDYMYGVSKVFGIDGGYRGFYSKNIINLTPKTVNDIHKRGGTILGSSRGGHDTTKIVDSIQDRGINQVYIIGGDGTQKGAAVIYEEIRRRDLKVIVAGIPKTIDNDIPVIDKSFGFDTAVEEAQRAINAAHVEAESAENGIGVVKLMGRYSGFIAMYATLASRDVDLCLIPESPFYLEGDGGLFEYIEKRLKENGHMVIVIAEGAGQELLAEENAHAKNEQDASGNKLLQDVGLWISQKIRDHFAKKTKMPITLKYIDPTYMIRAVPSNASDNVYCTLLAQSCVHGAMAGYTGFTSGLVNGRQTYIPFNRITEKQNMVVITDRMWARLLSSTNQPSFLRVKDIEEIKKEEQPQTQLLDGDNNVHENSGH

SEQ ID NO: 103 - protein, 289 aa, Solanum lycopersicum
MGNANAREDGAAGDGDGDGQVSGRRSNVESGIVEDHHALNSRVPSADLMVNSPPQSPHRSASPLLFGPQVPVVPLQGGDGNPVSNQMWGNECEDASDHSLEGGIPTLITWSYGGNNVAIQGSWDNWTSRKILQRSGKDYTVLLVLPSGIYHYKFIVDGEVRYIPELPCVADETGVVFNLLDVNDNVPENLESVAEFEAPPSPDSSYAQALMGEEDFEKEPVAVPPQLHLTVLGSENSEEAPSSPKPQHVVLNHLFIEKGWASQSVVALGLTHRFQSKYVTVVLYKPLKR

SEQ ID NO: 104 - protein, 598 aa, Solanum lycopersicum
MCTKFVKEMEKMALYVVVLLIICYASPARSDGSDHKYKSGDQVPLYANKVGPFHNPSETYRFFDLPFCTPDHVKEKKEALGEVLNGDRLVSAPYKLDFLYDKDSEIVCKKKLSKEEVSQFRSAVAKDYYFQMYYDDLPIWGFLGKVDKEGKSDPSEYKYYLFKHLHFEIFYNDDRVIEINARTDPNALVDITEDKEVDVDFMYTVKWKETKTPFDKRMEKYSQSSSLPHHLEIHWFSIINSCVTVLLLTGFLATILMRVLKNDFVKYAHDEEAADDQEETGWKYIHGDVFRYPSHKSLMAAALGSGTQLFTLTIFIFLLSLVGVFYPYNRGALFTALVVIYALTSGIAGYTAASFYCQLEGTNWVRNLILTGALFCGPLFLTFCFLNSVAIAYSATAALPFGTIVVIFLIWALVTSPLLVLGGIAGKNSRAEFQAPCRTTKYPREIPPLPWYRGTLPQMAMAGFLPFSAIYIELYYIFASVWGHRIYTIYSILFIVFIILIIVTAFITVALTYFQLAAEDHEWWWRAFLCGGSTGLFIYGYCLYYYYARSDMTGFMQTSFFFGYMACICYGFFLMLGSVGFRAALFFVRHIYRSIKCE

SEQ ID NO: 105 - protein, 601 aa, Solanum lycopersicum
MCTKFVKEMEKMALYVVVLLIICYASPARSDGSDHKYKSGDQVPLYANKVGPFHNPSETYRFFDLPFCTPDHVKEKKEALGEVLNGDRLVSAPYKLDFLYDKDSEIVCKKKLSKEEVSQFRSAVAKDYYFQMYYDDLPIWGFLGKVDKEGKSDPSEYKYYLFKHLHFEIFYNDDRVIEINARTDPNALVDITEDKEVDVDFMYTVKWKETKTPFDKRMEKYSQSSSLPHHLEIHWFSIINSCVTVLLLTGFLATILMRVLKNDFVKYAHDEEAADDQEETGWKYIHGDVFRYPSHKSLMAAALGSGTQLFTLTIFIFLLSLVGVFYPYNRGALFTALVVIYALTSGIAGYTAASFYCQLEGTNWVRNLILTGALFCGPLFLTFCFLNSVAIAYSATAALPFGTIVVIFLIWALVTSPLLVLGGIAGKNSRAEFQAPCRTTKYPREIPPLPWYRGTLPQMAMAGFLPFSAIYIELYYIFASVWGHRIYTIYSILFIVFIILIIVTAFITVALTYFQLAAEDHEWWWRAVIRLWWINRLVHLWLLPVLLLCPIRHVRFHANLILLWLHGLHLLCLLSHAWNCWFPSCFVFRPPHIPFNQVRVX

SEQ ID NO: 106 - protein, 485 aa, Solanum lycopersicum
MALLVEKTTSGREYKVKDMSQADFGRLEIELAEVEMPGLMASRAEFGPSQPFKGAKITGSLHMTIQTAVLIETLTALGAEVRWCSCNIFSTQDHAAAAIARDSAAVFAWKGETLQEYWWCTERALDWGPGGGPDLIVDDGGDATLLIHEGVKAEEEFAKNGTIPDPSSTDNVEFQLVLTIIKESLKTDPLKYTKMKERLVGVSEETTTGVKRLYQMQANGTLLFPAINVNDSVTKSKFDNLYGCRHSLPDGLMRATDVMIAGKVALVAGYGDVGKGCAAAMKQAGARVIVTEIDPICALQATMEGLQVLPLEDVVSEVDIFVTTTGNKDIIMVDHMRKMKNNAIVCNIGHFDNEIDMHGLETFPGVKRITIKPQTDRWVFPDTNSGIIVLAEGRLMNLGCATGHPSFVMSCSFTNQVIAQLELWNEKSSGKYEKKVYVLPKHLDEKVAALHLGKLGAKLTKLTKDQADYISVPVEGPYKPAHYRY

SEQ ID NO: 107 - protein, 123 aa, Solanum lycopersicum
MAAYAAMKPTKPGLEEPAEMIHKIRITLSSKNVKNLEKVCADLVRGAKDKRLRVKGPVRMPTKVLNITTRKSPCGEGTNTWDRFELRVHKRVIDLFSSPDVVKQITSITIEPGVEVEVTIAES

SEQ ID NO: 108 - protein, 338 aa, Solanum lycopersicum
MANGKIKIGINGFGRIGRLVARVALQRDDVELVAVNDPFISTDYMTYMFKYDSVHGQWKHHELKVKDEKTLLFGEKAVTVFGIRNPEDIPWGEAGADFVVESTGVFTDKDKAAAHLKGGAKKVVISAPSKDAPMFVVGVNENEYKPELDIVSNASCTTNCLAPLAKVINDRFGIVEGLMTTVHAMTATQKTVDGPSMKDWRGGRAASFNIIPSSTGAAKAVGKVLPQLNGKLTGMAFRVPTADVSVVDLTVRLEKEASYEDIKAAIKEESEGKLKGILGYTEDDVVSTDFVGDSRSSIFDAKAGIALSKNFVKVVSWYDNEWGYSSRVIDLICHMAKA

SEQ ID NO: 109 - protein, 230 aa, Solanum lycopersicum
MASNSAFSLFLILLIITQCLSVLNAAKDFDFFYFVQQWPGSYCDTKQSCCYPTTGKPAADFGIHGLWPNNNDGTYPSNCDPNSPYDQSQISDLISSMQQNWPTLACPSGSGSTFWSHEWEKHGTCAESVLTNQHAYFKKALDLKNQIDLLSILQGADIHPDGESYDLVNIRNAIKSAIGYTPWIQCNVDQSGNSQLYQVYICVDGSGSSLIECPIFPGGKCGTSIEFPTF

SEQ ID NO: 110 - protein, 487 aa, Solanum lycopersicum
MVDTLTELCSKLAGLMFILGTIQQLVPHSFYKRIKTLWHRIENYFYPYVQITIDEYSNYKRNEVYSLIKVYLGTKSTNDAKYLKAEMFKKSKFLAVSLDEGEEVIDEFHGVKLKWNSYTETFPDNSSGGRSQLPIEKKCYTLTFNKKNQEMVTGQYLKHVMEEGKAIEFKNKKQKIYSNEEGERYWYGKGMWRNINFEHPATFDTLAMDPTKKEEIINDLVAFSKGKDYYSKVGKAWKRGYLLYGPPGTGKSTMIAAIANYLNYDIYDLELTSVKDNSELKKLLMETTSKSIIVIEDIDCSIDLTGKRKKKNKKETTNEEEEEDSDSTTEKKNDKNESSKLTLSGLLNFIDGIWSACGQERIIIFTTNHVDKLDPALIRRGRMDMHIEMSYCKYEAFKVLAKNYLGIETHPLFQEIQSLLEEVDVSPCDVAENLMPKNASGNPEICLESLVKVLIKAKRKASLNSHKNKKSSTKLIITYGRFKKLFQ

SEQ ID NO: 111 - protein, 128 aa, Solanum lycopersicum
MQIFVKTLTGKTITLEVESSDTIDNVKAKIQDKEGIPPDQQRLIFAGKQLEDGRTLADYNIQKESTLHLVLRLRGGIIEPSLMALARKYNQEKMICRKCYARLHPRAVNCRKKKCGHSNQLRPKKKIK

SEQ ID NO: 112 - protein, 145 aa, Solanum lycopersicum
MPKNKGKGGKNRKRGKNEADDEKRELVFKEDGQEYAQVLRMLGNGRCEAMCIDGTKRLCHIRGKMHKKVWIAAGDIILVGLRDYQDDKADVILKYMPDEARLLKAYGELPENTRLNEGIANGLDEEDEGAGDDYIEFEDEDIDKI

SEQ ID NO: 113 - protein, 534 aa, Solanum lycopersicum
MQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGDL

SEQ ID NO: 114 - protein, 457 aa, Solanum lycopersicum
MQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGMQIFVKTLTGKTITLEVESSDTIDNVKAKIQDKEGIPPDQQRLIFAGKQLEDGRTLADYNIQKESTLHLVLRLRGGF

SEQ ID NO: 115 - protein, 393 aa, Solanum lycopersicum
METFLFTSESVNEGHPDKLCDQVSDAVLDACLAQDPESKVACETCTKTNLVMVFGEITTKANIDYEKIVRDTCREIGFVSPDVGLDADNCRVLVNIEQQSPDIAQGVHGHLTKRPEEIGAGDQGHMFGYATDETPELMPLSHVLATKLGARLTEVRKNGTCSWLRPDGKTQVTVEYHNDNGAMVPLRVHTVLISTQHDETVTNDEIARDLKEHVIKPVIPEKYLDENTIFHLNPSGRFVIGGPHGDAGLTGRKIIIDTYGGWGAHGGGAFSGKDPTKVDRSGAYIVRQAAKSIVANGLARRCIVQVSYAIGVPEPLSVFVDTYGTGKIPDKEILNIVKENFDFRPGMISINLDLLRGGNGRFLKTAAYGHFGRDDPDFTWEVVKPLKWDKPEA

SEQ ID NO: 116 - protein, 199 aa, Solanum lycopersicum
MNDPYRSKPSSGRTNFASCICATVFLIFLIAGIVVAYFFLFKPKTPKIAVEAVRFPTFSVTNGTVNFTFFQYVSVTNPNRDEFTHYDSSLQLSYSGEPVGLVFIPAGKIDGGRTQHMSAKFDVQSYPLPAKLKADVSGGMIPTTAVNVFDEGPTMEVETRMKLVGRVRVLKVFTHRVDSGVKCGVMIQVSSGSVLGVRC

SEQ ID NO: 117 - protein, 457 aa, Solanum lycopersicum; identical to SEQ ID NO: 114

SEQ ID NO: 118 - protein, 199 aa, Solanum lycopersicum; identical to SEQ ID NO: 116

SEQ ID NO: 119 - protein, 534 aa, Meloidogyne incognita
MRSSTLFVLAVVLLAVIVQLHADPKAGQKPAATKEKAGEGKPTAKVSSDQNKGKPKGQASNKIAHAAKEGAQHVKDAVSKLSHGNKPEVIGDDKLKTEIKTVLDGMIKHPEKIKQGNKAKLVKAVTCQVCKKINQDKNPKPKDQKLSKNQKRELKKEKKEKTKKLNAQIKKLLNSDAALKKEMDDLFNAVKKAHEKKAAALPKTNSGHLKKPAIAHPAVPKNAQAKPGHINVKQAHGQHNTNQAAALPISKSGPLKKLAGLKADLHKIPGHLSGKLHTTQHVSGTKSAETPPKHSPINFENLLRNVSSPLARKNVENKLPNSSNPSVPGALPSNMFGGKRKTEMDSEEDDWDVGDVDDFEEFSNRPIGNKGLETSGGKLSFPLKLAEDDYDNFEDDSLGDDSFDNDSLGDVDEDGDLPNSSLGKAESDENGKGASEEDKSDELDVDDFKENELNSNLLKTDKDKKLLSEGNGDLDEDLKKPSNEEVRKATELLQGAFSGLQKNIESSKVNSALPMNDSLVKQIGNPNLVDPVGA

SEQ ID NO: 120 - protein, 150 aa, Meloidogyne incognita
MKSFIFFLFIALTIYSTTFAIPIKDNDRPEFSLKNEENENAGKELILQKSKKDSKNVVNRATCYDNNLATYINNGLYYYPQNMGQLSNYILDRIKSARYSGYWFVHAAIITGQTQGLHWQSSATGDIFSPTSRHGCYYHDSYTYVVILKY

SEQ ID NO: 121 - protein, 361 aa, Meloidogyne incognita
MRFDIFLLVAFSLGVAVNCGVVKRNQNSYGDEAGAGGAAGAAPAAPAEAAAPSGGEAAPAAAPAPEAAPAAAPAPEQAAPAVAQPAPAAAASQASGYRKKRSQNSYGDEAGHAAGAAPAEAAPAPAESAAPSGGEAAPAAAPAPEAAPAPEAAPAAAPAPEQAAPAVAQPAPAAAASQASGYRKKRSQNSYGDEAGAGGAAGAAPAESAAPAGGEAAAPAATGCTENCAAGGESAAPAAAPAPEAAPAAAPAPEQAAPAVAQPAPAAAPSQASGYRKKRSQNSYGDEAGHAAGAAPAESAAPSGGEAAAPSGGEAAAPAPEAAPAPAPAEAAPAAAPAPEQAAPAVAQPAPAAAPSQASGY

SEQ ID NO: 122 - protein, 532 aa, Meloidogyne incognita
MKLFVLLIGVLAFTVPKQQIQQVEYKQFPQPYKFVPISNIVKCDRKTKECSVKLENLDPTNNYSLYTAKNDKGRGDKVKLTKVADIDLDKCQLDKNVKPEVNGEEICNQIVKGIDDNAKAETIEVNSGEIEFGSELEGTEDYAIVEKAMNEKKEHKNQQAVEHVHIPGPGEQPVEHNQPTIEYPTNSKQVHPADKYQHKLEERAKKFGLSDFKHGDLYEDYRQQKTVQEDEKDKRYQKVLGTLGDHKHPSLVDQYNEDKGKFNQRVKSDPTGNKVEKAKNSDSNGLEQKLEKLALSDFKHGDLYKDYQQQITVRDDEKDKRYQKVLGTLGDHKHPSLVEQYNRDKGKFNQRVKSDPTSNWHEDLFGKDYQRAMSDFDHLKAKQREKILGTLEDHKHPSLIDQYNKGSLNQRAKSDPTGNNIGKAKNSNFNGSEQKLEKLALSDFKHGDLLGRKGGIKQRTINVLAGKKIEHEKGSDFNANVEEMIGAENGKANQVNPKLTGRKLAEFNHIPAVDRILGFKRGGHALEEPHKN

SEQ ID NO: 123 - protein, 437 aa, Meloidogyne incognita
MSPSSFTLTAVLLEAIVFLYNRQVAAMLSMHPSCSGRSSTIENKLKMSGGGNGINKFTPGNVSFPVACQYHSKNLKATNKKEYKISEDLPMNQEKLTNSKEDDLIHKVKKIDKGNGAAVPYKTNKNNEIGDGAENGKAVKIREIIFTEEQKKMTSEEFEHYLYSVPYDKNKKNKIGKNENGEKVDKPNKEGGDTMFYSKAGIIAKKIKEYVPTNGEFKIQTGLVYRNNSFNASQDDSKNLLNISHILMALNENERDSQENLRNAADLFVALHECYQLFSAIPLVFEVEMVLKKLEEEGNKDDPIKLLEYFRLPTIKYPLLDLIKIENSTVSPDELIENVTKTIHKADNFIAKNIHAFFINDNETFFNEIISRLETADMVLASIKKILKMFNNFNEKIPENFSMLKRLKPIEMHDLFENSKLLQKLHAAILPGDEMKF

SEQ ID NO: 124 - protein, 576 aa, Meloidogyne incognita
MKSKAAAILMALMALYAEVVFGKALPIREIYSDLPPECKTLNLETVESIPTVITVNGKQYNIKGQIQHGATSRVFHAFEQHDNSPKRCMALKVVDLEKIEHKQRNQATNEAKILGELNQKKVERIPKLFEIEQHPTNKTISMVMELAHHDLFDEITPHMGLGKHEWVRNTFKEILQAINPLHKIGFVHGDLKPENMAYFRAPRVINDQHFPTKDDPLVLKVIDFGGVMKKQELSKACGEIVTGTVMYMSPEQVAWCAAHDEEEFLARRKQFPLSTKSDTWSAGVILYQMLHGKSPYYWITKKYEHLDEEERGEKADAELRQTIFDGKKPIEFDEKYQDLPLLEIVKATVRCLARNPDDRPEIEQLLHEMDKLGHRVEQLEGKIEKLEEEAEDLREEKEEREEEKERDEEEKKDEEEEDEKYEEEREEEEHRLAEERMKKQAQENARLQQEARFRYEQEMARRRQAQQANLQFFWNNFLGGMNNGYGFFPLRNQFQNNQVGQNFYPNNNNNNFAPPRMPQLAMPPPLNVFLPQTMSPLPPPKPVQTQQQAYPQTQKFIFPGLIEHLKEIHAIRASEK

SEQ ID NO: 125 - protein, 385 aa, Meloidogyne incognita
MKFLIAVFIGFVAFEFCLAEDVSTSGYDEVPIDDLLPVLPPFCSDVNLNKNEKVANCCNAIIRESKIVVYNTTVGPVPNECSLSKCGRVCNRYFSQRRPESNTTEEPGCECQKKQTTKKPKHSCQSEPEASTPPPRNPYQSGPEPSTQQPGGQVASHKPPSGPAAPSSRPPRNRYPTEPETKPPRNPYQSGPEPSTQQPGGIRQRSQASTPPPRNPYQSGPEPSTQQPGGQVASHKPPSGPAAPSSRPPRNRYPTEPETKPPRNPYQSGPEPSTQQPGGQVASHKPPSGPAAPSSRPPRNRYPTEPETKPPRNPYQSGPEPSTQQPGGQVASHKPPSGPAAPSSKPPRNLYQSGPEPSTKPPRNLYPSGPEEQPGATTESEIVRL

SEQ ID NO: 126 - protein, 65 aa, Meloidogyne incognita
MMKLNLIFCILVLIAIIFYVNEIEARAKYADPSDVQGCGTGCCVGVDTVTCDTGKQSCNCDLCTC

SEQ ID NO: 127 - protein, 316 aa, Meloidogyne incognita
MYFKAFLLLLISLLSAFVVNNARTISNMDEVGDVSATMTEKKNNLGEKSNQVLPQGEEEMDDLTEEYDDSDDSEEDEDNDDEEDDMDEDNDANNYDYLNSRIDHMDDIDSFETYEELPHMDNYGKMNEAKQMMPVMHMNAGENEHYKQLPSIATQPINEANPIMPPMPVVQPEKMEQAPEAMKTEATPQASQPSSPKTAAPKAKTSKPKTELKKPSAKKVQATKGAAKVTKKDAKKPKDVKKSKDPKAKTAKGKVPKAKAAKGKPAKGKPAKPGKGKPLKGKTAKASKAGSKKQPAKKGGKKSGSKKVTAKGGKKH

SEQ ID NO: 128 - protein, 100 aa, Meloidogyne incognita
MRQQIIENNLLSLAFLLFFLISLINTINSKPHPNGLPMVRRDGNGGDLDDLLTEFRAKGSRMRFGKRSSSSSPRFSSSSADVDFIEAPIYYIPDRFMWFQ

SEQ ID NO: 129 - protein, 482 aa, Meloidogyne incognita
MLKAASMFYSMIPFLILLCSLFENCGAGVPHAAVSSSQGVNIPHKISASMTKLGNPRVTHVTFPVKQNSSKSGISLTVSDPKQPVLRASAKHDNQDLFSNDENNDDDELQYYIKKYEEKERDEVARINLEDVEEHNKYMEEQEKLYEQTKGKEGLDKISKNLLDRKHASYSKISSYELIKEFYGIDLEYKIPKTIEQRKNLVTKLLEGFIKMINKRNKEKFDIMISALGNENHKEKKGEFDEFLQYLELFAPDTILVSFLQIPDEGRQLLFETFDEILTDLKKSKNKKDEHGKIIKGSSQHPAVHLKKYHDKLNNSDLDADTKERIDQFFTIVQNDFNQLPIYLTENFASLFRKIATLGLPGLKEALIRYLEVWNHADPQDQQMMANAFPKLAFFYNDSKSMKMNDWAYRSVKGNFFFKIPLLILLKMIFLNKPKLENDSALYKELREKLTKDFEANKELIDEIGKFFKPAKPLVGSVKILN

SEQ ID NO: 130 - DNA, 2730 bp, Solanum lycopersicum
  source 1..2730: mol_type = genomic DNA
atggctgaagaagaaacaaatggagcagcagcagagggtctgccacctcctccccctgttccaccagatttcactcctgctaaaactgaaattgagcctgtgaagaaaaagatcttgcgcgttcccatggccaggcgtggtgttggaaataagggacaaaagattcaaatacttactaatcactttaaagtgaatgtaaacaatgtggatggacacttcttccactacagtgttgccttattctatgaagatggtcgaccagttgatgggaagggagttggaaggaaggtgttagacacagtgcacgaaacttatgatacagaattagctggaaaggactttgcctatgatggtgaaaaaagcttgtttaccattggagcactccctaggaataaaatggagttcactgttgtccttgatgacgtgatatcaaataggaacaatggtaacagcagccccggtgggcatggtagtcctaatgaagctgacaggaagaggttacgtcgtccttaccagtcaaaaactttcaaggtggagatcagttttgctgccaaaatcccgatgcaggcgattgcaaatgctctgcgtggtcaagagtcagagaactctcaagaagccttgcgagtcttggatattattttgaggcagcatgctgcaaagcagggttgcctgcttgttcgacaatcctttttccacaatgatcccaagaactttgttgatgtcggcgctggtgtgcttggttgtcgaggattccattccagcttccggactacccagagtggcttgtctttgaacattgatgtgtctacaacaatgattatccagcctgggcctgtcgtggacttcttgattgcaaatcaaaatgcaaaggatcccttttcactggattgggcaaaggcaaaacgtgtgctcaagaatttgagggtgaagacaacccccactaatcaagagtacaagattactggactgagtgaccgtccttgtcgtgagcaactatttaccctgaagcagaaaggaaaagatgcagatggtgaggtccaaactactgaagttactgtgttcgattactttgtcaaccaccgcaacatagagttgcgttattctgctgatttaccctgcataaatgttgggaaaccaaagcgtcccacctttttcccaattgagctatgttccttggtgtctttgcaaagatacacaaaatccttgtccacattccagaggtcttcactagttgagaaatcaaggcaaaagcctcaggagaggatgcaagtcttaagcaatgctctcaaaatcaaccaatatgatgctgagcctctgcttcgttcctgtggaatatccattagcaataacttcactcagattgaaggacgtgttcttcctcccccaaagctgaagacgggcggtgatgactttgtccctcgtaatggcagatggaatttcaataataagagactagttgatcccactaagatagaacgctgggccgttgtcaatttttctgcacgctgcaatgtacaagggctagtcagtgatctgatcaaatgtggaaaacagaaaggaattatggtggaagacccatttgatgtctttgaggagtctccacaagtcaggagggctccaccacttgtgagggttgagaagatgtttgagcaggttcaatccaagctacccggggcacctaaattcctgctttgtttgcttcctgagaggaaaaactgtgatgtatacggaccatggaagcgaaagaatcttgctgagtatggcattgttacccagtgtatagctccaacaagagtcaatgatcaatatatcaccaatgtgctcctgaagataaacgcgaagcttggaggtttgaactctatgttaactgttgaacattctcctgcaatccctatggtgtctaaggtccctaccattattcttgggatggatgtgtcccatggctctcctggccaatctgatgtcccttcaattgcagcggttgtcagttcaaggcagtggccatcaatatctcgttacagagcttcagttcgtactcagtctcctaaagtggagatgatagacaacttgtttaaacgtacctctgatactgaagatgatggaataatgagggaggcgttgctagatttttatgtgagctctggaaaaaggaagcccgagcatataataatattcagggatggtgtcagtgaatctcagtttagtcaagttctgaatgttgagctggaccagattattgaggcttgtaagtttcttgatgagaagtggtcacccaagtttgtggtgatagttgcccagaagaaccatcataccaagtttttccagcctaatgatcctaacaatgttcctccaggcacaatcatagacaacaaagtctgtcacccaaggaattatgacttttacctgtgtgctcatgctggaatgattggtacaactcgtcctactcactatcacgtcttgtatgatgagctgggtttctctgctgatgatcttcaagagcttgttcataatctctcttatgtgtaccagagaagcaccactgcaatatctgttgttgctcctatctgttatgcccatcttgctgctactcaaatgggacagtggatgaagtttgaagatgcatcagagacttcctcaagtcataatggagtaaccaatgctggaccagtctccgttcctcagcttccaaagctcgaggagaaagtttccagttctatgttcttttgctaa

SEQ ID NO: 131 - DNA, 3981 bp, Solanum lycopersicum
  source 1..3981: mol_type = genomic DNA
atgtcttctatttgcccacaaaattggaagaatgatgttttcttgagttttagaggtaaagatacgcgtaaaacttttgttggtcatctcaactatgctctaaaacaaaaaggaattcatacattcaaagatgatgaaagattggaaagaggaaaaaccatttcacctgaacttgttaaagctattgaagaatcaagatttgctattgttgtattctctaagaattatgcatcatccacttggtgtttagatgaacttgtgaagatcatggaatgtaaaaaagaattaggacaaactgtgatacctatattctatgatgtagatccatcacatgtaactaaacaaagtgaaacttttgctaaatcctttgctatacatgaggaaaatttgaaagatgatgttgagaaggtgttatgctggagggatgcatttcgtcaagcaggcaaaatagctggatatgatttgccaaatggctatgacgggtatgaatctaattgcattcagcatgtagttgaaggcatactgagtaaattatgtcaagttatttcaaccatagaaaatgatttggtggggatggaatctcgagtgcaggaagtaagttcattactaaggatggaaacactcgatgttcgttttattggaatttgggggatgggaggcattggtaagacaacaattgcaagagctgtgtttggcaaatattggggccaatttgaaggagtttgttttctggataatgttgtagaaatgcaaaagacacatggactgcaatatctgcaaggtgttctcctctcgaaaatcctcaaagtaagtataactatagcaagtgtgtatgaaggcatgagaatcataaaggaaagactacgctcgatgaaagttttgatcattcttgatgatgtaattcaaaaggaccaattagaaatgttagttggagggcgtgattggtttggtatcggcagtagaattttgattactacaacagataaacatttgttacataatcatgtggtggatgaagtgtattcagtcaacttgatgactcgcgatgaagcgatagagctattcagcctacacgcctttaagcaaagaattccagataaagattttgaggagctattaaatcaagttgtacagactgccggttttctccctttagctctgaaaattctaggttcgtctctctgcgggctagacaagattgtgtggaaatgcacagtggacagacttaaggatatgccagatgatgttcttgctaagcttaagataagttttgatgggctgagtccttctgatcagagactatttcttgatatcggatgcttttatagaggaaaattgaggagttctgtaggggaaatgcttgagagttgtgatattggatctacaataagagtcttgattgaaaagtctctcttatttgtctcaccatatgacacaatcgaaatgcatgatttgatacaagaaatggcctggcacatcgtgagacaaggcgacacaagaaggagcagaatatggcttcccgaggacatcgaggatttgtttactggaaatttggaggcagaagctgtggagggcctatcgatcccaagggattacgtacgaaaacgggatatatcatgttacaacataaatgaaacattcaggaaaatgaaaagattaagggtgcttgaagtaagagcaacaaatttctgctctattgatccgatcactcatcttcctagcagcataaggtggcttgattgggaaggttgccctttaaattcattgccacagagttttgaaccttcaaagcttcttcgtctcgatatacttgaaagtactacactccagaagctctggtcaatttcaaagggattggacaaacttaaggctttgtacctcagctactgtgagaagttggaagaagttccaagcttcgatataatgccaaatttagagagagtaaagctagagggatgtaacagtttgagagaagtcagcccatcttttggagttctcatgaaactcacttcaatggagctaattgattgtccgagcctcaagaaaattccaagttgtgttgagatgggatcccttaagagtctcaaactttcttgtcttccaaaattgagggaattgccagaaaccaaggggatgcaccgtttattgacgttagagctgactaactgtcaataccttgagaagcttccaaattttgatcagatggaatcccttgagactctcaaactttcttgtcttccaaaagtcactatatttccgccaccagacggaatgcactgtttacaggacattgttatcgaatatgtcccgatagtagagcttccagcatcaatcgggaatcttcgttccctcaaacaacttagattaagtcattgtaaagatctagtaagcattccaaacagcttttgttgtctgaagaatctaagagcccttcttatctacaattgcaaaagactaatctatttgccagaaaagattggtgacttgaaactgttaaagaagctagtagtatctggtacttcaatttcccagataccaacttcagttacagatcttcgtgagctaagcttcttatcgttttctcgttggtctggatacagacaaggtgcaacttttctgttcccgccttcatcaggtgtatcgttgccgttgagggtgttaaagctaaaaaaaaacaggctatgtagtgcagaacattttccagatgttggaggtttatcttcgttagctcacttggatttgactagaaatgatgagagtaccaatcaactctttcattacttagacataacattttgtgagaagcttgcattgcctgaacttccgccatttataaaggagttatatgcatgtgatcctctaatcttgaaaagcatccctgatttccttaccaaatattcagatctctattcagtgtcattcactcgccatatcgagaacaaaggtgaactgactgatttattgctttttgtcctccgcatgattagtgtggcatctcagtgtgagaaaaggccgcccttgagccttttcttccctggagatataaaatggaaatggttcacttattatcggaaagaacaaacgaaaagattctccactcaacttgatccacgttggtacgaaagtaaattcatcaaaggatttgttttatgctttcgtgtaccatcggtgattggtcagaaccagaaatgttcaaatgataaatcacaacgaggtagtagtcgtaggttcggatgcactaaggttactgccaagttagtgcaaagatataacaggcaagaacatgatgtactccagaaaaaatgtctgattgttgctcgccaaactttttgctctcatggtagtaaatactccatttgttttaactacataccttttgtagcatgtgagaaagggaagaagctgctaaatgagtattgcttcttcgaggcatcgttagaaccagacattgcaacaaaatgggggcttcttctggtgtatgagaagaaaattcaacagatagatcaatcaaccatcgcggtccaatgtgatgttgattctccgaattctgatctgtttagagaatgtaatgatgaccaaggccacagaaaagaagatgtttctgttaagagaagacgccttgatcttcgtcaagtgatgaaggattcgtgctccaccagcatagttcctgatcaacaaatggaaacaccaagctcttctgcagcacaaagcttccagcatagggatgagtcatgctcttcaggacagccccaaactacacagatccctcaagctgatgatccgcgagttgatgaagcaatagatgaggctacctcatgtgtggtagttgaccagcctgacgtttttgatgtctctccagatgagcacaaagataattcaatgacaactggttctggctgctctgtagtatcccaacaattggagacacaatgcacttctggaaaatctcaaactctccggctcttccccgatcattcatga

SEQ ID NO: 132 - DNA, 420 bp, Solanum lycopersicum
  source 1..420: mol_type = genomic DNA
atgggagtggtgattatcgacggatcaacaattcgagatttcgtcaacgacgaagcggctttcacaaagtcaattgataagggatttaccgatttggacctcaataacgacggcgttttgtcacgatccgagctccgcaaagccttcgaatctctccatctcattgaatctcacttcggcgtcgacgtcgccgccacgccggaggaactcaccgacctctacgattccattttcgagaagttcgattgcgaccacaacggcactgtcgaccgagaggaatttgggaacgagatgaggaaaattatgcaggcgatcgctgatggacttggttcatcgccgattcaaatggcgcttgatgatagtgaacagagtttgattaagcaagctgctgatctcgaagcttctaagcttcctgcctaa

SEQ ID NO: 133 - DNA, 912 bp, Solanum lycopersicum
  source 1..912: mol_type = genomic DNA
atgcctactattgctaaacatgatggtcggcagctggagactgggctgcgaattacctcactatcaactacccaaatgcaaccatggtggcacggttatggtgataatactatgcctttggctagtgagaatgccgttgcccaggagaaatctgaaggtggcaaccaggataaagaaacaaaggctctagccatggaatctggatcagatggaaataacgagcaatacaagcagcacctcaagcattttgcacccacaactgctgccatcatggctgagcagcagaaggaactcacaggtcattcagctatgttggcgtcatatccatatccagatatgcagtatgggggaatgatgacttatggcgcacctgtacatccccacttatttgaaattcatcatgcccgaatgcctttgcctctcgacatggaagaggaacctgtctacgtgaatgcaaaacagtaccatggaatattaaggcgaagacagataagagctaaagcagagcttgagaggaaagctattaaagcgagaaagccatatctgcatgaatctcggcatcagcatgctatgagaagggcaaggggaactggaggccgctttctgaatactaaaaagctcaatgacatggactgtacaccaacggagactcaaaaatatggggcaaccattgcaactcactctggcaactcttctggttccgggagttctgatcaagggggaaaggaaggatctacagttcaagacatgcacaaaggacacagccagagtttcacaactggcaatggccatggttcatctgtgtatttttccgcgagcagtggaagtgagcaagggaatggccattacggtcatggaagttggagcctgctggtcaatcaggcttctcagggggcagcttccagcaattaa

SEQ ID NO: 134 - DNA, 570 bp, Solanum lycopersicum
  source 1..570: mol_type = genomic DNA
atggtgtgcatacgaaaggcgacgatagatgatttgcttgctatgcaagcatgcaatttgttatgccttcctgagaattaccagatgaagtactacttctaccacatactttcgtggcctcagcttctgtacgttgctgaagattacaatggtaaaattgtgggatatgtgttagcaaaaatggaagaggaaaccactgagtgccatgggcacatcacttcccttgccgtagtcaggactcaccggaaacttggtttggctactaagctcatgaccgctgctcagaatgctatggaacaggtgtttggagcagagtatgtgtctctgcatgttaggaagagcaacagggccgcatttaagctgtatacagagacattaggatataagattcatgatatggaagccaagtactatgcagatggggaagatgcttatgatatgaggaaggaattgaagggcaagaagcaccaccagcatcaccatcaccaccaccaccacggtggtgggtgttgctctggtgaggtgaaggtaggaccaaaggaaggagcagcagaaacaaaagcagcataa

SEQ ID NO: 135 - DNA, 1167 bp, Solanum lycopersicum
  source 1..1167: mol_type = genomic DNA
atgtattgtggtagcattttaacagtgtctaatttcaattatttgtgaattttattcttaattattaaattaatggaatattggggttcaattgaaaagcctattgttttgttgggttgtgcatatttgtcatggaagattgttacgcatatatggagtttcatgaatatagaaaaggatcccattacagtgcttgttactggtgctgcaggtcaaattggctatgctcttgttcctatgatcgcgagaggagccatgttaggccttgatcaacctgttatcatacacatgcttgatattgaaccagctgctgagtccttaaaaggagtgaaaatggaattgattgatgcagcttttcctcttctcaaagatgttgttgctaccacgaacgttgttgaagcttgtaaaggcgtcaatgttgctgttatggttggtggatttccgcgaaaagaagggatggaaagaaaggacgtgatgacgaaaaatgtttcaatttacaaagcacaagcctctgccttagaacagcatgctgcccctgattgcaaggttttggttgttgctaatccagcaaacactaatgcacttattttgaaagaatttgccccttcatttccagcgaaaaacatcacttgcctaacaaggcttgatcacaacagagctttaggtcagatttcggagaaactaaacgttcacgttggtgatgtgaagaatgtggcaatatggggaaatcactcatcaactcagtatccagatgtgaatcatgcaactgtgaaaaccgctgctggagaaaagcctgtcagagaacttgttgctaatgatcaatggttgaacacggaattcatcaccacagtgcaacaacgaggagccgctatcataaaagctcgaaagctatcaagtgcattatctgcagctagctcagcttgtgatcatatacatgattgggttctcggcactccaaaggggacatgggtttcaatgggggtttattctgatggatcttatggcattccagctggtttaatttactctttccctgttacttgtgaaaaaggagaatggtcaattgtgcaaggtctaaagatagacgagttttcaagggccaagatggatgcaaccgcgaaggagttggctgaggaaaaatccttagcgtattcatgcctaaactga

SEQ ID NO: 136 - DNA, 1428 bp, Solanum lycopersicum
  source 1..1428: mol_type = genomic DNA
atgaatgtcatgcgtcgccttaagagcattgcttctggacgttcttctgtttcagatcctggtggtgattcaagcataaagagggtgaaggttgagaaagaagtagatcaaagagtggttggtgaaactcaaatggaagagggatgtacaactactacagttccaaaggaagacatggcttctacatctatggaaaccactgctggaagtacctcaacaatggatattagacttgaaaattctgaacttgatgaacttcctaaagaaatgcatgaaatgaaaattaaggatgaaaaagatgataaagctgatagtctagatgataatttgaaggatatggaacctgctgttgttagtggaaatggaacagaaactggtcagattattgtgactactgtgagtggtaggaatggacaagagaaacagacattgtcttacatggcggagcgcgtggttggcactggttcatttggagttgtctttcaggctaagtgcttggaaactggtgaatctgttgcaataaagaaggtcttacaggataggagatacaagaacagggaacttcagattatgcgcacacttgatcatcctaatgttgttaaactacgacactgcttctattctactactgagaagaatgaagtctaccttaaccttgtcctggaatacgtgtctgaaactgtttaccgagtatcaaggcactacagcagaatgaaccaacacatgcccaatatatatgtgcagctatacacataccagatatgccgtgctctgaattacatgcatggtgtccttggtgtatgccatcgtgatattaaaccacagaatcttttggttaatccccactcgcatcagctaaagctctgtgattttggtagtgcaaagatgttggtgcctggagagcccaacatatcatacatttgttctcgttattatcgggctcctgaattgatctttggtgctactgagtacacaactgcaattgacatgtggtctgctggttgcgttatggctgagctacttttgggacagcctcttttccctggagaaagtggcgttgatcagctggtcgaaatcatcaagatattggggacaccaacaagagaggagattaggtgcatgaatccaaattacacagagttcaagtttccccagatcaaagctcacccatggcacaagatatttcataaaagaatgccccctgaagcagttgatttggtgtcgaggcttctccaatattctccaactctacgctgcactgctttggaagcatgtgcacaccctttctttgattctttaagggaaccaaatgcttgcttgccaaatgggcgacctctgcctcccctattcaacttttcacctcaagagctctctggcgtgcctgctgaactgcgaaagcgccttattcctgaacacttgcgcaagtga

SEQ ID NO: 137 - DNA, 435 bp, Solanum lycopersicum
  source 1..435: mol_type = genomic DNA
atgccgaagaataagggaaagggaggaaagaacaggaagagagggaagaacgaagctgatgatgaaaagagagagcttgtttttaaggaagatggacaggaatatgctcaagttcttcgtatgcttggaaatggccgatgtgaagctatgtgtattgatgggacgaaacgtctatgtcacatacgtggtaagatgcataagaaagtttggattgccgccggtgatatcatccttgtcggactccgtgattatcaggatgacaaggctgatgttattctgaagtatatgccagatgaggcaaggttgttgaaggcatacggagagttaccagagaacactaggctcaatgagggtattgctaatttggatgaagaagatgagaatgccgctgatgattacattgagtttgaagatgaagacattgacaaaatctaa

SEQ ID NO: 138 - DNA, 561 bp, Solanum lycopersicum
  source 1..561: mol_type = genomic DNA
atggaatcacagtttattgaaagataccatagtcatcagcctagtgaacatcagtgttcttcatctcttgttaaacacatcaaagcaccagttgatattgtttggtcactagtgaggagatttgatcagcctcagaagtataagccatttattagcaggtgtactgtgaagggtgatcttacaattggtagtgttagagaggtgaatgtcaagtctggtcttccagccaccactagcactgaaagattggaacttcttgatgatgaggagcacatccttggcatcagaatcgttggtggcgatcacaggctaaagaactattcttcggttattacagtccatccagagacacttgatggtagaccggggacactggtgattgagtcatttgtagtggatgtgcctgaagggaacactaaagaggagacttgctactttgtgaaggccttaattaattgcaatctgaaatcactagctgatgtatctgagaggatggctatgcagggaggtgttctacccgtcagtgtgaactggtcttcgagtaaccagatcgagatatga

SEQ ID NO: 139 - DNA, 372 bp, Solanum lycopersicum
  source 1..372: mol_type = genomic DNA
atggcagcatatgcagcaatgaagccaaccaagcctgggcttgaggagcctgcagagatggttcacaagattaggatcactctctcctccaagaatgttaagaatctcgagaaagtgtgcgctgatttggttcgtggtgccaaggataagaggctcagggtgaagggaccagtgagaatgcccacaaaggtccttaacatcaccactagaaagtctccttgtggagaaggtacaaatacatgggacagatttgagcttcgtgtccacaagcgggtgattgaccttttcagctcaccagatgttgtgaaacaaatcacttcaatcaccattgaacccggtgttgaggttgaggtcactattgctgaatcttag

SEQ ID NO: 140 - DNA, 621 bp, Solanum lycopersicum
  source 1..621: mol_type = genomic DNA
atggacggcggcagcagcaacgcaccagttcagcagctgacggcggaggcagctgcacagctacaggaaggtatcggtttggtgttaactagatggactgctcttcagatggctatcgagaacgagtggggtggccgagatactcgtgagaaatccaatcaactcaatgttgacattttttccgcttttacacaatccaaagaaaaagtatacatggatgatatagaagaaattctagatgagttcatgatatctctaaatactgaagtcaatgatggcagccttgaggaggtagcagaaaaattgatgtatatgcatgaagagtgtctggaaggggatttcaattcaatcaaagtgctaagggaaacaaatgttgggaggagacctgctacttatgtcagacaagatgctagtgatgacgatgacagtagtaatgatggggatgaccgaaatgagaactttggcaataattcatccgatatggcaattgattctatggaaagccagccaagtttgggtcaggatatgatagttgaacccgtgagaaagcagccagctgaagtggacccagacgggtggacgacagtatctacaaagcgtaacaaaggtagaaggaattaa

SEQ ID NO: 141 - DNA, 1032 bp, Solanum lycopersicum
  source 1..1032: mol_type = genomic DNA
tcatctatcagctctcctctctctcacaggcacacaaagagacaaaaactcaaaaacaaagaaaattaacagaaaagaaaaatggcagaggctaattcgtatgcagctcttaccaaagctttctcaggacttggtgtggatgagaacttattcatctcaacactggggaaatggaatcggcatcagagggaatcgtacagggtaagcactcctggattcttcagagaagacaaacgacagttccagagatgggatgatcaacatatcctccagcttcgtcaagagtttttgcgtcttaaggatgcagtggttttgtacacaatgcacccatgggaaagagatgctcgtttgttcaaggaagcattattattgcaagtacctaaacatgatgttctcattgagattgcttgcactcgatcctctgaagatcttcttggagccagaaaagcttaccattccctttttgaacactcaattgaggaagacattgccttccatgttcgaagttctgaaagaaagcttctagttgctcttgtaagttcctaccgttatgaaggtccacgagtaaacgatgatcttgcaaaatctgaggctaagatttttgtaaatgcaataaaaaatgccaacaagaagaagctgattgatgaggatgaagagattgtgaggatcctttcgacaagaagcaagctccatcttaaggccatctatagccattacaaggaaatcactggcaacttcttggacgaggatcttgagtgtgacttgaccttgaaacaagttgttcaatgcctttgtatacctaaagcatactttagcaagattttgatcgcgtccctaagattggatgtggatgaatctgctaaagactcagttacccgagtcatagtgtcaagagcagacgatgatgatatgcagcaaatcaaagaagaatttcagagcaaatatggaactactttggctgaaaagattgcagaagttgctaatggaagctacaaggatttcttgcttactataattgcaaaatctgagtaa

SEQ ID NO: 142 - DNA, 633 bp, Solanum lycopersicum
  source 1..633: mol_type = genomic DNA
atgggtttatcattcgggaagcttttcagccggctgtttgccaagaaggagatgcgtattctgatggttggtcttgatgcagctggtaaaaccaccatattgtacaagctcaagctgggagagattgttaccaccattcctaccattggattcaatgtggagactgttgaatacaagaacataagcttcaccgtctgggatgtcgggggtcaggacaagatcagaccattgtggaggcattacttccaaaacacacaaggacttatctttgtggttgatagtaatgatcgtgatcgtgttgttgaggccagagacgagctgcatcggatgttgaatgaggatgaactgagagatgctgtgctgcttgtgtttgctaacaagcaagatcttccaaatgctatgaatgctgctgagatcactgacaagcttggtttgcactccctccgtcaacgtcactggacaggtacattcagagcacatgtgctacctctggagaagggttgtatgaggggcttgactggctctcaaacaacattgcaaacaagggttgaacacgagatccatgatttttgcagctatatttttgtcaatatagacttcaaacaatgtctagagcacttgtgcacctgctga

SEQ ID NO: 143 - DNA, 1182 bp, Solanum lycopersicum
  source 1..1182: mol_type = genomic DNA
atggaaactttcttattcacctccgagtctgtgaacgagggtcacccagacaagctctgtgatcagatctctgatgcagttcttgatgcctgccttgagcaagatcccgagagcaaagttgcatgtgaaacttgcaccaagaccaacttggtcatggtctttggtgagatcacaaccaaggctattgtagactatgagaagattgtgcgtgacacatgccgtaatattggatttgtttctgatgatgttggtcttgatgctgacaactgcaaggtccttgtttacattgagcagcaaagtcctgatattgctcaaggtgtccacggccatctgaccaaacgccccgaggagattggtgctggtgaccagggccacatgtttggctatgcaacagatgagacccctgaattaatgcctctcagtcacgtgcttgcaactaaacttggtgcccgtcttacagaagtccgcaagaatggcacctgcgcctggttgaggcctgatggcaagacccaagttactgttgagtatagcaatgacaatggtgccatggttccaattagggtacacactgttcttatctccacccaacacgatgagaccgttaccaatgatgagattgcccgcgaccttaaggagcatgtcatcaaaccagtcatcccagagaagtaccttgatgagaatactattttccaccttaacccatctggccgattcgttattggtggacctcatggtgatgctggtctcactggtcgtaaaatcatcatcgacacttatggtggttggggtgctcatggtggtggtgctttctcgggcaaagacccaaccaaggtcgacaggagtggtgcatacattgtaaggcaggctgcaaagagtatcgtagctagtggacttgctcgtagatgcatcgtgcaggtatcttatgccatcggtgtgcctgagccattgtctgtattcgttgacacctatggcactggaaagatccctgacagggaaattttgaagatcgttaaggagaactttgacttcagacctggaatgatgtccattaacttggatttgaagaggggtggcaatagaagattcttgaaaactgctgcctatggtcactttggacgtgatgaccccgatttcacatgggaagttgtcaagcccctcaagtgggaaaagccccaagactaa

SEQ ID NO: 144 - DNA, 1806 bp, Solanum lycopersicum
  source 1..1806: mol_type = genomic DNA
atggcgttgaagttatctgctgctgaagcagctaaggttgcgattcagtctataggatgtggatatgatatatcattggatttgaggctcaagtattgtaaaggtcatccagataatagtcgtttaattgaaattgatgaaaatgagagtggggaggttgtgcttcctggtggaatttcaatacctaatgtgtcaaaatctatcaaatgtgataaaggagaacacacacggtttggctctgatgttctttctttccagcagatgtcagagcagttcaatcaggaagtatccgtgaatggtaaaattccttcggggctcttcaatactatgttcgagttttctggttcttggcagaaggatgctgcatacactaaaacccttgcttttgatggtgtgttcatcacgttgtactctgttgctctagaaaaatctcaaatggtgctgtgtgattatgttaaaaaggaagttccatcaacatgggatcctgcagcattagcaaggtttattgaaaaatttggtactcatgtcattgttggtataaagatggggggaaaagatgtaatatatatgaaacaacagcactcctcatcgcttctgcccgctgacgtgcaaaaacgtttgaagacaatggcagataagaggttttcaggtgcaaatgaacaagggggagtagaatcgcagcagacacatcagaatgagaagtttgaaatcagggagcaccgactgaggtttgccgatcccaatatatcaggttcatatgcacataaggaggatactgtttgtatttgcaagaggagaggtggaagtgatagcagaggcctgatgcataatcagtggttacaaagtgttgatcaggagcctgatgtgatctcaatgtccttcattcctataacttcactgttgaatggcatctcaggaagtggatttttaagtcatgctataaatttgtacttacgctataaaccaccaattgaagagcttcaccagtttttggaatttcagctgccaaggcaatgggcaccagtattcggtgatcttccgcttggtccacagagaaggcatttaagttttccgtctttgcagttcagtttactgggtcccaggctttatgtgaacaccactccggttgatgttggcaagaggccggtgactggacttcgtttgtaccttgaaggtagaaggagtaaccgcttagccgtccatttacagcatctttcatctcttcctaaagtcttccaacttgaagatgatccaaaaggcaactttcgccgagaatcatatgatcgcaaatactatgaaaaagttcaatggaagaacttttctcacgtttgcaccgcacctgttgaatctgatgaagaccttaacatagttactggagcacaattggaattaggggattatgggtttaaaaaggtcctctttctgagactccgtttctcgactgtcatgggagctactatggtaaagcacgctgagtgggatggttcccctgggttggctcgcaaatctggcctgatatcaactctcattagccagcactttacatcagttcagaagccacctcctcggccagatgatgtgaacataaattctgccatttaccctggtggtcctccagttcctgtgcaagcccctaagctcttgaaatttgtcgatactactgagatgactcgaggaccacaggaacctcctggttattgggttgtttcaggtgcaaggcttatggttgagaaaggaaggatctctgtgagggtcaaatattccttgctaactgtaatacaacccgatgatgaaatatcagagtaa

SEQ ID NO: 145 - DNA, 999 bp, Solanum lycopersicum
  source 1..999: mol_type = genomic DNA
atggcgtattacaattacaatttaatcaacaaaatggttacaattatttttattctggtgctagtaattgttgatgtgactatggtattcggccaagggactcgtgttggattttattccagtacgtgcccaagggccgaatccatagttcaatcaacggtgaggtctcattttcagtctgatccaacagtggcaccaggattgttgagaatgcactttcatgattgtttcgtacaaggttgtgacggttccatcctcatttctggtactggcactgagagaacagctcctccgaattccaatttgagaggattcgaggttattgacgatgctaagcagcaaattgaagctgtttgtcctggagttgtttcatgtgctgacattcttgctcttgctgctcgtgattccgttctcgtgactaaaggattgacctggtctgtccccacgggacgcagagatggacgagtttcaagcgcatcagacacatctaatctgccaggttttactgaatctgttgatgctcaaaagcaaaagtttgctgcaaagggtctcaacactcaagatcttgtcacccttgttggtgggcacacaattggaacctcagcatgccaattcttcagctacaggctatacaatttcaactccactggtggccctgacccttcaatagatgcaacctttctttctcagcttcaagcattatgtccacaaaacggagatggctcgaagcgtgtggcactggacactggaagcgtgaacaattttgacacctcgtatttctctaacttgaggaatggtcggggaattctggaatcagaccagatattgtggaccgatgcttcgaccaaggtgtttgtccaaaggtatttaggcctcaggggatttcttggattgagattcggcttagaatttggaaagtccatggttaaaatgagcaatattgaagttttgacagggactaatggtgaaattcgtaaagtttgctctgcattcaactga

SEQ ID NO: 146 - DNA, 1062 bp, Solanum lycopersicum
  source 1..1062: mol_type = genomic DNA
atgctagcaaatcagaaatcaaaagctgtgacttatcgaaagcttgcaaggagttaccatccagatgtgaacaaagaacctggagctgaacagaaatttaaggagatcagcaatgcgtatgaggttttgtctgatgatgagaaacgttccatatatgataaatatggggaggctggtcttaaaggagctggtgcgggcatgggggattttagcaacgcctttgatttattcgagtctttgttcgacggctttggtggtatgggtggcatgggtggtatgggaggtagaggttctcggagcagggccacagaaggtgaagaccagggctataatctggttttgaatttcaaagaagctgtatttggggttgagaaggagattgaaattagcaggcttgaaacttgtggtacctgtgatggatcaggtgcaaaacctgggactaaaccgtcgacctgtaatacttgtggtgggcaagggcaggttgtgtcctcagcaaggaccccactgggtgttttccagcaggtgacaacatgctcttcttgtggggggactggagagatttctactccttgcggcacctgcaacggtgatggccgagtgaggaagtcaaaacgcattagcttgaaagttccacctggtgtagattccggtagccgtttaagggttcgttcagaaggtaatgcaggaaggagaggtggaccccccggagatctttttgtcatgattgaagtcctcccggatccagtattaaaacgggatgacaccaatattctctacaattgcaaagtttcttacattgatgcgatattgggtaccactatgaaggttcctacagtagatgggatggttgatctaaagattccggcaggtacccagccagggacaacattggtgatggccaaaaaaggggtgccattcctgagcaaaccaaacatgagaggggatcaattggtgagagtgcaagttgagattccaaaacggttgagtagtgaagaaagaaagctcatagaagaacttgccaatctaaacaagcctaaagctgctaccaacagcaagaggtag

SEQ ID NO: 147 - DNA, 528 bp, Solanum lycopersicum
  source 1..528: mol_type = genomic DNA
atggccgacgttgaatacaggtgcttcgtcggtggtctggcatgggccaccaccgaccaaacactttcggaagctttttctcagtacggcgaagtggtcgaatccaagatcatcaatgaccgggaaactggtagatctagaggatttggattcgttaccttcaaggatgagcaagccatgagggatgctattgaagggatgaacggccaggatcttgatggtcgcaacatcaccgtgaacgaagctcaatcacgcggaggcggtggaggtggaggtggaagaggcgggggtggttacggaggtggccgacgtgaaggcggtggcggaggctacggtggtggcggcggctacggaggtggccgacgtgaaggcggcggcggcggctacggtggaggcggctacggaggtggtcgtcgtgaaggtggttacggtggaggcggtggtggttatggaggaggtgaccgctacaacgatcgctcttcaagaggtggaggcggtggtggttccgatgggaactggaggaattag

SEQ ID NO: 148 - DNA, 1071 bp, Solanum lycopersicum
  source 1..1071: mol_type = genomic DNA
atggatttttcatattcttccccttcacatttcattcttagtttatgttttgctctcctcattttatccagaggtgttttaggggctacatttacatttgtgaacaaatgtgagtacacagtatggccagggattctagcaaatgcaggtagtccaactctgcaaagtactggatttgagcttcctcaagagtcttcacgtgtattcacagcacccactggttggtccggtaggttttggggacgaaccagttgtacgtttgagggatccgggtcgggttcttgcggaacaggggattgcggctccggtgaggtagaatgtaacggtgctggagctgctccgcctgctactctagcggagtttacattgggtactgttgggcaggatttctatgacgtgagtttggtggacgggtacaatttacctatggtggtagaagcgtcgggcgggtcgggtatgtgtgcgacaacgggttgcgtaacggatctgaatcaaatctgcccgtcggagctgaggagtggcggtggagaggcatgtagaagcgcgtgtgaggcgtttaacaagccggagtattgctgcagcggcgcgtacaatacaccgtccacttgtaaaccgtcaatgtactcacagatgttcaaatctgcgtgtccaagatcatacagctacgcttatgatgatcctaccagtactttcacttgcagtggtgcagattataccatcacattttgtccctctaccccaagtcaaaaatctacaaaagatactactacccccaatggtgatgggtcatatggtgctactggtacaggagttgtggaccctgcaacaggaggaatcgagtcaagttcgggtcaaatgggaaccggatcaggcaccgggtttgagtttggtgcaggaacaggatatgggtcaggaaccgaaacacagcctggatcgggttcacaagctatgctagcagatggatcctatttagctggactggctatgggagaatcaagtagggcattttttccattgaatttagcaggcagtgctttggctacaactttggtttcattagttttcttgtag

SEQ ID NO: 149 - DNA, 750 bp, Solanum lycopersicum
  source 1..750: mol_type = genomic DNA
atggctagatacgacagagcgatcaccgttttctcgccggacggccacttattccaagtggagtacgccatggaagccgtacgcaaaggaaacgccgccgtcggtgtacgtggaactgacactgttgtcctcggcgtcgagaagaagtccactcccaagcttcaagactccaggtcagtaagaaagatagtgaatctagatgatcacattgcgttggcgtgtgctgggctgaaagcagatgcacgagttcttgtgaataaagcacgcattgagtgtcagagtcataggcttacagttgaagatcctgttactgttgagtacataactcgttacattgctggtcttcagcaaaagtacacccaaagtggtggggtgaggccatttggtctatccaccttgatcattggttttgacccacacacaggtgtcccctcactttaccaaacagatccatcaggtacattctcagcttggaaagccaatgcgactggtagaaactccaactctactcgggaattcttggagaagaattacaaagaaacatctggccaggaaactgtgaaactagcaatacgtgcattgcttgaagttgttgaaagtggtggaaaaaacatagaagttgctgtgatgacaaaagagcatgggcttaagcaacttgaggaagctgaaattgatgccattgttgctgagatcgaagcagagaaagctgctgcagaagcagccaaaaaggccccaccgaaggaaacctag

SEQ ID NO: 150 - DNA, 1620 bp, Solanum lycopersicum
  source 1..1620: mol_type = genomic DNA
atgggttttgtgagtacgattttgggtttttgtggatttggagttggggtttcatgtggattgacgatcggttactatctcttcatctatttccaaccctgtgatgttaaggaccctgttattcgtccattggttgagcgagattctaaaagcttgcagcagttgctgtctgaaattcctctctgggtcaaatgtccagattatgaccgtgtggactggctcaacaaatttatcgagtatatgtggccttacctggacaaggcaatttgcaggactgcaaaagatattgcggcaccaattattgctgaacaaataccgaagtataaaattgattctgttgaatttgagacactaactttggggtccttaccccctactttccaggggatgaaggtctatgttactgaagaaaaagaattgatcatggaaccatcaataaagtgggctggaaatcctaatgttaccgttgcggtcaaagcattcggattgaaagcaactgttcaggttgtggacttgcaggtttttgcagctccacgtattactctgaagcctctggttccaagctttccatgttttgccaatatctttgtgtccctcatggaaaagccacatgttgactttggattgaagcttttgggggctgatcttatgtccatccctggcttgtacaggtttgtccaggagactattaaagatcaggttgctaacatgtatctttggcctaaatctctagaagtgcagatattggatccatccaaagcaatgaaaaaacctgttggggtcctgcatgtgaagattctgagggctatgaatctgaaaaaaaaagatctactgggtgcatctgatccttacgtaaaacttaagctcactgagtcaaaacttccttcaaagaaaacccctgtaaggcataagaatttaaacccagagtggaatgaggaatttaatatggttgttaaagatccagaatcgcaagcattggagctctctgtttatgattgggagaagattggcaagcatgataagatgggcatgaacgttattccactgaaagatttgactcctgatgaaacaaaaacgatgacactgagtctcctaaagaacatggatgcgaatgattctcaaaatgataaggaccgtggtcagatcatggtggaattaacctacaaaccatttaaggaggatgagttgccaaaagattttgaagataatgatgcagcacataaggttccagaaggaacaccaccagggggaggcgtccttatgattattgtccacgaagctcaagatgttgaaggaaagcaccatactaatccatatgtcaagattcttttcaaaggagaggagagaaaaactaagcaagtcaagaagaacagggatccaagatgggaagaggagtttacttttgtgttggaggagcctcctgtgaatgatagggtgcatatggaagttgtcagcacctctacaaggattggcctattgcatcctaaggagacattgggttatgttgatataaatctttccgatgttgttagcaacaaaaggatcaatgagaagtaccacctcattgattcaaagaacggtcgccttcaagtcgagctgcaatggcgaaccgcatcttga

SEQ ID NO: 151 - DNA, 804 bp, Solanum lycopersicum
  source 1..804: mol_type = genomic DNA
tacagcaagaaaataaatatgtctctcttcttccttgtcttgcttcttttcgtctcttcagttacagcctgtgatcgctgtgttcaccgatccaaagttgcttatttttccagttcttccgcgcttctctcgggtgcttgtggttatggaaatttagcaataggattcaatggaggtcgacttgccgctgctgttcctaaactttacgataatggtgctggttgtggtgcttgttatcagataagatgtacggacccaaaaatttgttcaaaacatggaacaactgtgatggttactgatctaaatacgaataaccagactgattttgtcatcagtagcagagcttttatggctatggccattgagggaaaagctcaagatgttcacaaattgggaatcgccgatgttgaatacaaaagagttccttgtgactacaaaggcaagaatttggctgttcgagtagatgaatcaagccagaagccgcattacctagcaattagcatcttctaccagggtggtcaaactgaaattgtcagtgttgatgttgctcaggttggatcgtcgaactggaacttcttgagccgaaaccacggagcaatttgggatacaagcagagtgccaagtggggcattgcaattcaggtttgtagtgacagcagggtacgatggcaagtggatttgggctaaatcagtgctgccatcagattggaaaaatggggtggtttatgatactggacttcaaatcactgacattgctcaagaaggttgttccccatgtgatgatggaagttggaaacttaattag

SEQ ID NO: 152 - DNA, 1890 bp, Solanum lycopersicum
  source 1..1890: mol_type = genomic DNA
atggcatgtatgaagttgggatccaaaacagatgcattccaaagaaaaggccaggcctggttctgcacaactggcctcccaagtgacattgttgttgaagttggggaaatgactttccatctccacaagttcccattgctttctagaagtggggttatggaaaaacggattgcagaagcatctgaaggagaagatggctgtgttattgaactcaatgacatccctggtggtgctaaaacatttgaactagtagcaaaattttgttatggggttaaactagagcttacagcaggtaatgctgtataccttcgctgtgccgcagagcatcttgatatgactgaagaatatggggaggggaatctgatatcacagactgagatttttctgaatcaggtagttctccgtagctggaaagactctttaaaggcactccaaacctgtgatgatgttcttccatatgctgaagaactcagaattacaaaaaggtgcatcgattcattagctgttaaggcatgtactgatccgaacctgtttggttggcctgtgatggagcacgttggccccttgcagagtcctggagggagtatcctatggaatggaataagcactggagctaggccaaaacattcaagttcagattggtggtatgaggatgcatcaactttaagtttacctctttacaagaggctaatttctgctatggaatctcaaggcgtcaaacaggatattgttgctggttccctcagttattatgctaaaaagtacctgcctgggctaaacaggcggcagacctcttctgagtccactaaccgacttgcaccagttggtttaggttcttctctatcagaagaagatcagaagcttttacttgaagaggtggataatttacttcccatgcaaaaaggcctagttccaactaaattcctctttggcctactgaaaacagctttgattcttcgagcctgtccctcttgcatatcaaacttggagaaaaggataggaatgcagcttgatcaggccactctagaagatctcttaatgcctaatttctcttactcgatggaaacactttataatgttgactgcgtacagagaattctagagcacttcttagccatggatcaaggaactggtggtgaatcaccttgttccattgatgatgagcaattgattggttcaccatctctgacaccaatcactatggtggcaaaactaatcgatggatatcttgctgaggttgccccagatgttaacttgaagcttcccaaatttcagactcttgctgcttctgttcctgaatatgcaaggcccttggatgatggcctttatcgtgcaattgacatttatctcaagtcacatccatggttgggagaatctgatagggaacaactctgcaggctcatggattgccagaaactctccttggaagcttgtacacatgctgcacagaatgaacggctccctttaaggattatagtacaagtactcttctttgaacagctgcagttgagaacatccattgcaggatgcttcctagtgtctgaaaaccttgatgaggggtcaaggcagttaagaagtggcacggtgggaccaaatgaggggggctgggctactgctgtgagagaaaaccaggtgttgaaggtgggaatggatagtatgagggtgcgcgtgtcagagctcgagaaggagtgctcaaccatgaggcaggagattgagaagttgggtcgatcaaagggatcaagcacttggggaaatgtatccaagaagtttggtttcaagatgaaatctcaaatgtgcagtgctcaggagggatctgttagtaaccagaacaagataaatagtaaggctataaaggatgacaaggcaaaagaaaactatcgaaagcactag

SEQ ID NO: 153 - DNA, 1629 bp, Solanum lycopersicum
  source 1..1629: mol_type = genomic DNA
tttttttatggaccactttggcccaaaaaggtacactactggttagctgtagcagattctgtggaaggaaaacatcaaaggaaaatggacggaacagcagtgcagggcaccagcagtgttgactcatttttacggaactataaactcgggaaaacacttggcattggatcgttcggcaaagttaaaatagctgaacatacgttaacagggcacaaagttgctgtcaagattcttaatcgtcgaaaaatcaggaatatggacatggaggagaaagtccgtagagaaatcaaaatattgagattgttcatgcatcctcatattatacggctttatgaggtcatagagacaccatcagatatatatgttgtgatggagtatgtgaaatctggcgagttatttgattacattgttgagaagggcagattgcaggaggatgaagctcgtaacttttttcagcagataatttctggtgtggagtactgccatagaaacatggtggttcatagagaccttaagcctgaaaacctccttctggactccaaatggaatgtgaagatcgcagattttggtttgagcaatatcatgcgcgatggtcattttctgaagacaagttgcggaagcccaaactatgctgccccagaggttatatcaggtaaattgtatgctggccctgaggtagatgtatggagctgtggtgttattctttatgctcttctctgtggcacccttccgtttgacgatgaaaacatacccaatctttttaagaaaataaagggtggaatatatactctgcccagccatttatcagctggtgcgagggatttgattccgaggatgcttatagtcgacccaatgaagcgaatgactattcctgagattcgcctgcacccttggttccaagctcatttgccacgctatttggccgtgcctccaccagatacaacccaacaagcaaagaagatcgatgaagagattcttcaagaggtggttaagatgggatttgacaggaacaaccttactgagtctcttcgcaatagagttcaaaatgagggcactgttgcatactatctgctcctggacaatcgccatcgtgtttccactggctatcttggagctgaatttcaggagtccatggaatatggttacaaccggatcaattctaatgaaaccgctgcttcccctgttggtcaacgtttcccaggaataatggattatcagcaagctggtgcaagacagttccccattgaaagaaaatgggctcttggcctccagtctcgagcgcatccacgtgaaataatgactgaagttttgaaagctctgcaagaactgaatgtatgttggaaaaagattggtcagtataacatgaaatgtcgatgggttcctagcttacctggtcatcatgaaggcatgggtgttaattccatgcatgggaatcagttctttggagatgattcatccatcattgagaatgatggggccacaaagttaacaaatgtggtcaagtttgaagttcagctttacaaaaccagggaggagaagtacttgcttgaccttcagagacttcagggtccacaattcctcttcctggatctctgtgctgcttttcttgctcagcttcgagtactttaa

SEQ ID NO: 154 - DNA, 918 bp, Solanum lycopersicum
  source 1..918: mol_type = genomic DNA
atgtcgtcgaacataattcacgacgccgatggatcggcttcgcgaatcgtattgaaggagcatactggatcaactgcagaggtgctttttcagggtggtcaagttgtttcttggaagaatgaacgaagagaagaactgctgttcaggagtaataagaccctgtggaaacctcctaaagccttcagaggtggaatacctatcagcttcccacagtttggaacttttggttctttagagagacatggttttgcaagaaatagagtgtgggaattggacgattctccttctcctctacctgcagcaaacaatcagtcaactgttgatctcattttgaaatctaccgaagaggatctgaagacctggccgcataggtttgaggtgcggctacgcatttctctcagtgctggcaagctgactttgattcctcgtgtcaggaatatagataacaagccattctcattcacattctctctgcgtaattatttatctgtgtctgatatcagtgaagtgcgggttgagggcttggagacacttgactacttcgataatttgcagcggggagaaagatacacagagcaggcagatgcaattacatttgatagtgagacggatagagtatatcttagcacaccaacaaaaactgccataatagatcacgagaagaagagaacttatgtccttcagaaagaaggaatggcagatgctgttgtttggaatccttgggacaaaaaggccaagtctctcccagatttgggtgatcaagattacaagaaaatgttgtgtgtggattctgctgctgttgaaactccgattatcttaaaaccttctgaagagtggagggggcggcaagagctctctacggtatcttcaagttattgcagtggacaattggaccctaggaaagttatttatggctaa

SEQ ID NO: 155 - DNA, 615 bp, Solanum lycopersicum
  source 1..615: mol_type = genomic DNA
atggagatgcaaagcgaattcgatcgtctccttttcttcgaacacgctcggaagagtgctgaaaccgcctatgctcagaatcccctagatgctgataacttgacgaggtggggaggtgcattgctggaattatcacagtttcagcccgtagctgaatcgaaacaaatgatttctgatgcaacatcaaagttggaggaggcattgacagtgaaccctgaaaagcatgatgcgctttggtgtttgggaaatgctcatacatcacatgcttttttaactcctgatatggatgaggctaaggtttactttgaaaaagcaactcagtgctttcaacaggcatttgatgcggatccctcaaatgacctttaccgcaaatctctggaagttactgctaaggctccagaattgcacatggaaatccacagacatggtcccatgcaacaacctatggcagcagaaccttcaacatcgacaagcacaaagagttcaaaaaagaagaagagcagtgatctcaagtatgacatttttgggtgggtcatacttgccgttggcattgttgcatgggtgggatttgcaaaatctaatatgcctccaccaccaccaccgcatccacagtga

SEQ ID NO: 156 - DNA, 1140 bp, Solanum lycopersicum
  source 1..1140: mol_type = genomic DNA
cttgtatctataaatatattttctggggttcctccatttttaagccaccattacagaacccaagaaaaagggcccaaaaatatgaagcacatagccacttttttgcttttggtttctgtctccacccttgttttgcaggttgttgcagaaaatccaatatcccaagctaaagcggaatctgcaatccttcaggactcaatcgtcaaacaggttaatgaaaatgaaaaagctggatggagagctgcactaaaccctcaattctcgaatttcacggtttcccaatttaagcgccttcttggagttaagcccacaagaaagggtgatttgaagggcatccctattttaactcatccaaaacttttgaagttaccccaagagtttgatgcacgagtggcttggcctcagtgtagcactattgggagaattcttgatcagggacattgtggttcttgttgggcttttggtgctgctgagtcgttgtctgatcgtttctgtattcattatggattgaatatctctctgtcagcaaatgatatcatagcatgctgtggctatttatgcggggatggttgtgatggtggatatccgttagaagcttggaaatactttgttcgcaagggtgtggtcactgaagagtgtgacccttactttgataataagggatgttctcaccctggatgtgaacctggatatcccactccacagtgcaagaggaagtgtgttaaagagaatctactctggagtaaatccaagcattttggtattaatgcatacctgattaactccgacccatacagtatcatgacagaagtttacaagaatggaccagttgaggtttcttttactgtttacgaggatttcgctcactacaagtctggtgtttacaagcacataaatggtgaggaaatgggaggccatgctgttaagctcattggatggggaacctctgaggatggagaggactattggctccttgccaatcaatggaacagaggatggggtgatgatggttacttcaagatcagaagaggaacaaacgagtgtggcattgaagaagaagtggttgcaggaatgccttcagccaaaaacctgaacgtggaacttgatgtctctgatgctttacttgacgcctcaatgtga

SEQ ID NO: 157 - DNA, 1098 bp, Solanum lycopersicum
  source 1..1098: mol_type = genomic DNA
aattggaaagctttaaagaaagaaaaaaaaaacatggccttgactttgaagtcattaataactcctttgctttttggtgctttctttatcctgatcttacagcaggttgctgcagagaagccaataactgaagcaaaacttgaatctgcaatccttcaagactcgatcgtcaaacaagtcaatgagaatgccgaagctggatggaaagctgcattcaaccctcaattatcgaatttcacggtttcccaatttaagcgccttcttggagttaagcctgcaagagaaggtgatttggagggaattcctgttctaactcatccaaaacttaaggagttaccaaaagagtttgatgcacgaaaggcttggcctcaatgcagcacaattggaagaattctaggtcaggacattgcggttcttgttgggcttttggtgctgttgagtcgctgtctgatcgtttctgtatccattataatttgtatctctctgtctgtaaatgatctgttagcgtgctgtggctttttatgcgggagtggttgtgatggtggatatcctatagcagcatggcgatactttaaacgtaggggagtggtcacagaagagtgtgatccttacttcgacactactggatgttcccaccctggttgcgaaccactataccccactccaaaatgtcataggaagtgtgtcaaggggaatgtgttatggaggaaatcaaagcattacggtgtaaatgcatacagggtcagccatgatccccagagtatcatggcggaagtatacaaaaacggaccagttgaggtctcttttactgtctacgaggattttgctcactacaaatcaggagtttacaagcatgtaacagggggaaacatgggaggacacgctgttaagcttatcggatggggaactagcgagcaaggagaggactattggctcattgctaattcttggaatagaggctggggtgaagatggatacttcaagattagaagaggaacaaatgagtgtggcattgaacatagtgtggttgcaggattgccttcagcaagaaatttgaatgtggaacttggtgatgctgtccttgatgcctcaatgtaa

SEQ ID NO: 158 - DNA, 1026 bp, Solanum lycopersicum
  source 1..1026: mol_type = genomic DNA
atggggttttccgtctttttattcacctttctcttacaacaccttctaatattcaaccacgtgttctatctctgtagtaccgaccgttgttcaaatcgattcttcactttaccacgactgatctcactctctctcaatctgaacatggcggaagcaagtgcaccagctgtaccaagtactgagttgttggagtggccaaagaaagataagcgcagaatgttgcatgctgtatatcgtgtcggtgaccttgaacgtaccatcaagttttacacagaatgttttgggatgaaattgttgaggcagagagatattccagaggagaagtattcgaatgcttttcttggttttggccctgaagagtctcactttgtggttgagttgacatataattatggagttgataagtatgacatcggaaccggctttgggcattttgctattgccacaccagatgtttacaaactggttgaggagataaaggccaagggtggaactgtcacaagggagcctggtcctgtcaagggtggatctagtgttattgcttttgtcaaagatcctgatggctacctatttgaaatcatccagagagagtctactcctgaaccactttgccaagtgatgcttcgtgtgggggatcttgaacgtgcgatcaaattctacgaaaaggcacttgggatgcaagtggtgaagaaagttgatagaccagagcataagtataccattgctatgatgggatatgctccagagaaagagacgactgttattgagttgacatacaactatggtgtgactgaatacacaaagggaaatgcatacgcacagattgcaataggcactgatgatgtctacaaaagcgcgaaggttgtcaatcttgctatccaagagctcggaggaaagataactagacagccaggatcaattcctggacttaacaccaagataacttcatttctggatccagatggctggaaaacagtgctggttgacaataaggattttctgaaggagctagaatcgtccaagtaa

SEQ ID NO: 159 - DNA, 795 bp, Solanum lycopersicum
  source 1..795: mol_type = genomic DNA
atgctactactgtcacttttagtagcattcgcactcttttctccaaccccatttgtccattctcactacaactggtcaccttcttcttcctccacctccaattttcagtccgagtggcgtcctgcacgcgccacctattacgccgccgccgacccacgtgacgttgtgggtggcgcgtgcggatatggggatttggagagaaatgggtatggaaaagctacagctgggttaagtactgtgctttttgacaagggtcagatctgtggtgcttgttatgaagtgaggtgtgtggaggatctgcggtggtgcattccgggtacttctataattgttacagccactaatttttgtgcaccaaattatgggtttgatctagatggtggtggtcactgtaatccacctaatgcccattttgtgctacccattgaggcttttgagaagattgccatttggaaagcttctaatatgcccattcaataccgcaggataaagtgcagaaaggaaggtggagttcgattcactcttggtggctctggtatattcttgtcagttctaatcagtaatgttgcaggttcgggtgatatagtggcagcaaaagttaagggttcaagaacaggctggcttcctatgggtaggaactggggccaaaactggcacattaatgctgacttaaagaatcagccgctttcttttgagatgactagtggtgatggggccaccttaacatcttacaatgtagctcccaagaattggaattttggtcagacctttgaaggcaagcagtttgggtcttag

SEQ ID NO: 160 - DNA, 2538 bp, Solanum lycopersicum
  source 1..2538: mol_type = genomic DNA
atggaggttaactcacttcagaagtgggttctgctatggtgtatagtattgtttattagctctgggttggttcactgtgatgttacatatgataggaaagctattgtgattaatgggcaaagaagattacttttctctggttctatacattaccctagaagtacccctgagatgtgggaagatctgataaacaaggcaaaagaagggggtttggatgtggttgagacctatgtcttttggaatgttcatgagccttctcctggcaattacaattttgaaggaagatatgacctggtgaggtttgtaaaaacgattcagaaagcagggctgtatgctcatcttcgaattggcccttacgtttgtgcagagtggaattttggagggtttccagtatggctgaagtatgtacctggcattagcttcagagctgataatgaacctttcaagaacgcaatgaaagggtatgctgagaaaattgttaacttgatgaagagtcataatcttttcgagtctcagggtggtccaatcatactctcacagattgagaatgagtatgggcctcaagccaaggtacttggagcaccgggacatcagtattcaacatgggctgcaaatatggcagttggattagacacaggcgtcccatgggtgatgtgcaaggaagaagatgcaccagatcctgtgatcaacacatgcaatggtttctactgtgataatttcttcccaaacaaaccatacaaacctgcaatttggactgaagcttggagtggatggttctcggaatttggcggtccccttcatcagagaccagttcaggatttggcatttgctgttgcccaatttatacaaagaggaggatcttttgttaactattacatgtaccatgggggcacgaactttggacgcactgcgggtgggccattcatcactaccagctatgattatgatgctccaattgatgagtatggtttgatcagacagccaaaatatggtcatctcaaggagcttcatagagctgtcaagatgtgtgagaaatccatagtttctgcagatccagcaatcacatccttgggaaaccttcaacaagcatatgtgtactcttcagagacagggggttgtgcagctttcttgtcaaacaatgactggaaatctgctgctagagtaatgttcaataatatgcactataatttgcctccttggtccattagcatccttcctgattgcagaaatgtagtcttcaacacggccaaagttggagttcaaacatcaaagatggaaatgctaccgactaactcagagatgctatcttgggagacttacagtgaagatatatctgcattagatgacagctcatcaattaggtcttttggtctcttggagcaaataaatgtaaccagagatacaagtgattatttgtggtacataactagtgttgatattggttcaactgagtccttcttgcatggtggagagctccctactcttatagttgagacaacaggccatgctatgcatgtgtttattaatggtcagctctcaggttctgcatttgggacaaggaagaataggagatttgtatttaaaggaaaagtcaatcttcgtgctggctcaaacagaattgcactgcttagcgtggctgtcgggctgccgaacattggtggacattttgaaacatggagcactggcgtactaggtcctgttgcaattcaaggacttgaccatggaaaatgggacttgtcctgggcaaagtggacatatcaggttgggcttaaaggggaggccatgaatcttgtttcgacaaatggtatttctgccgttgactggatgcaaggttcattaattgcacagaaacaacaacccttgacatggcataaggcttacttcaatacacccgaaggagatgagccattggctttggacatgagcagtatgggtaaaggtcaagtatggattaatggccagagtattggtagatattggactgcatatgctactggagattgcaatgggtgccaatattctggagtttttcggcctccaaaatgtcaactgggatgtggggaaccaactcaaaaatggtaccatgttcctcggtcttggttaaaacccactcaaaatctgttggtgctttttgaagaacttggcggagatcccacaagaatttctctcgtgaaaagatcagtcactaacgtatgttcgaatgtagctgaatatcatccaaacattaagaactggcaaattgagaactacggcaaaaccgaagagtttcacctacccaaagttcgcattcactgtgctcctggtcaatctatttcctccattaaatttgcaagcttcggaactcctctcggaacctgtgggagcttcaagcaaggaacttgccatgctcccgactcacacgccgttgtagaaaagaaatgtctaggacgccaaacatgtgctgtgacaatatcaaatagcaactttggagaagatccctgccccaacgtactgaagcggttgtctgtagaagcacattgtacacctactcagaattga

SEQ ID NO: 161 - DNA, 1302 bp, Solanum lycopersicum
  source 1..1302: mol_type = genomic DNA
atgtcgacacttatttgtttagcaagtccgtcgacactggtgctaagtatctcaaagttgaaaaacccaaaaacagcaagtctttctgtgttatgttggacgaaggagaagaaatcattgatgaattcgaaggagctacattctgctggcgctctcacattgaaacgttcgatgataattctcaggggagtaaaaaaaaaaacccagcctagtcagaagaaaagttacataattacattcaataagcaacatcgagagattgtcatcggaaaatacttgaaacatgtgattgaagaaggcagggcaattgagttcaagaataagaagcagaagatttattgtaacgatagaatgccttcttggtgggcgtgttacaactcaatgtggaagcatatcaattttgagcaccctgcaacttttgatactctggctatggatcctaagaagaaagcggaaataatcgacgatctcattgctctcaccaacgggaaggattattattccaaagttggaaaggcgtggaagcgcggttatcttctttatggcccaccaggaactggaaaatcaactatgattgcagcaattgcaaattacttgaactatgatatttatgatcttgagctaacttctgtaaaggataacttagaacttaagaagttgctcatggaaataacaagcaagtctattattgtcattgaagacattgattgttccgttgatctcactggaaagagaaagaaggcaaaaaagaataagaagaaggatgactcagattccgactcagattccgattctgattcagactcagactcagactatgaaggcaaaaaaaaactcacactatcagggttgttgaatttcattgatggcatttggtcagcttgtggtgaagagagaattattatatttaccactaatcatgtggacaaacttgatccagctttgatacgtagaggacgcatggatatgcacattgagatgtcgtattgcagatacgaagcattcaaagtgttggctaagaattacttgggcattgatacacatcctttgtttcaagagattcaacctttactagaggaagtaaacttgagtccttgtgatgtcgctgagaccttgatactaaagaatacttcgggagggattgagatttgcttgaataacttgattctggctttgaaagattccaaggagaaggcaatacaagaggccagggagaaggaaaaacaagattcggaagaaaaaccaaagaagaataaaaagttaaaaaaaatttatggccgttttaaaaagcttttcagatga

SEQ ID NO: 162 - DNA, 1062 bp, Solanum lycopersicum
  source 1..1062: mol_type = genomic DNA
atgtctctcaagattaatctgttgatctgtttcttcattcttcatctatgggctgctacaggacacaagataggaatttatgagatcaagaaaggagatttctctgttaaggtcactaattatggtgccagaatcatctctgttcttcttcctgataagaatggaaaaataggtgatattgttcttggatatgacaccattggggaatacgtgaacgatacgagttcctttggagctattgttggaagagttgttaaccggattgctggtgctcgatttactctaaatggaaccctttataagcttatcgccaatgatggcaagaacacaatacacggtggccctaaaggatttagccatgttgtttggaaggtgagcaagtacaagaaagatggtcctcatcctcacattactttaacctaccacagtactgatggtgaacaaggatttcctggtgatcttcttgcctctgttacctatgcattggaagacccttacaagcttagtgtggtaatgaaagcaaaagctctaaacaaggccactccagttaacttggctcaacatacttattggaaccttggtggacacaacagtggcgatgttttgtccaacgatattcaaatctttgcttcacacatcaccccattagatcaacatttcattccaacaggaaaaatcgcccctgtcaagaacacgccctatgatttccttaaaccccgtaaaattgggagccagttcaagaaactccaaaatggatatgacataaactatccactagacagcactgaaaaactgaagcccacggcaacagttcacgataataagtcaggaagagtgatgagtttacaaacgactgcacctgctgtccaattatacactgcaaacacccttgtaaagaaagggaaaggtggatttgtctatcagccttattcagccgtgtgtttggagactcaaggattccctgatgctgtgaatcacccaaatttcccatcaacaattgtgactccggaacatccctactttcacaaaatgctgtttacgttctcaattaagaaatattag

SEQ ID NO: 163 - DNA, 1233 bp, Solanum lycopersicum
  source 1..1233: mol_type = genomic DNA
atgacgactgtggttcctacttctgaggaagatcccgtcctctccgtcgtccggtttacggcagagatgtcatgggcggacgccggcgccgagactgcagagcctgaagttaatagattgtgtgtcgaggcccagaaatgcatgattgaaggaaggtggttggatttggtttcgcttatgctcgcttctgcagatataatattttctaaagcttctgaaaaagatcttgagtgcgttttcacaataatgtgcaaccttgtcaagaagcctgagagcttggaccaagtacatgagatggcagagctcatatccgctaaaatcattcaacaacccaatgataaacccgcattgcgcttgaaaatcttgttcaatctctacaatctgttggagaacccatacagccgtttctgcgtttacttgaagtccctcaagttggccactgccgggaaggttactgaacacattttgtcgtccatcaaaatgatggataacttcttgaaggagtggaatcttggagtcaaagatcagagagagctgtttcttgcaatctccaacattttaaaggaaagcaaaggctctacaaaagattctttcatgtttcttacaaagtatctggaaaccttttcgtctgaagatgcttctaatatgactgaggctaaggaagaagctgccagtgcaattgtcgattttgtcagatcacctgatatgtttaagtgtgacttgctagatatgccggctatagtccagttggagaaagatggtcaatatggtccagtatatcaacttctgaagatctttctcactcagaggctggatgcttacttggaatttgaggcagcaaattcagcattattaaaaacctatggacttgtccatgatgactgtatatcaaagatgaggttgatgtcgttggtagatcttggcttgaatgaatctagtcaaattccttattctgtcatcaaacaggcattgcagattgatgacattgaggtagaatcgtgggttgttaaggcaattacagctaagttacttgattgcaagattgatcaaatgaatcaagtagtaatagtgagccgatgtacggagcgtgtgtttggtgtgcatcagtggcaagatctccgcacgaaactagtgacttggaggggcaacattgctggtgttatcagcacagttcaagccaataagattacagaagatagcacacaggcagtgcaaggtttagcaatccgctga

SEQ ID NO: 164 - DNA, 1101 bp, Solanum lycopersicum
  source 1..1101: mol_type = genomic DNA
atgttaagctcctgttttgggagctgctttggtggggacgatggtgatgatcttttatggcatatggacttgaagccacatgccacaggtgaatattctgttgctgtagttcaggccaattctttgcttgaagatcaaggtcaagtttttacaacccctagtgctacttatgtgggtgtttatgatggtcatggtggcccgcaagcttctcgcttcatcaacaacaatctcttccctcaactacaaaagctggctttggaagaaggaggattgtctgaggacgtgatcaggaaagcatttgatgctattgaggagggattcttgcgtttggtgaaacagtcttggctggatcaacctcagattgcatctgcagggtcttgttgtctatttggagcaatctcaaaagatgaattgtatgtggcaaatcttggggactccagggcagtgcttggcaggagagcagagacaggccagacgggtgattcagcagtagtagtggcagaacgtttgtcgatagatcataatgttggggttgaggaggttaggaaggaggtagaggcactgcatccagatgattctcatgttgtggtctacgttcgaggggtttggagaattaagggtataattcaggtttctagatcaattggtgatgtctatctgaagaaacctgagtttaatagagatcctatgttcatacaatatggatatcctattcctttaaaaagagctgtaatgtcagcagagccctccatattgatccgaaagataagaccagaagacttatttttgatatttgcatcggatggcctatgggatcaactaaccgatgacgaagctaaggatattgttttgaagaatcctagaattggaatagcaaaacgattagtgagagctgcccttgagggtgctgcagagaagaaagaaatgagatatgaggacatcaaacgactagagaagggggaaaggcggcacgttcatgatgatatcacagttattgtgatatatttggatcaccctggaaggtcctcaaattctggaattaacattgttaagggcacgaatgtccctcttgacatatactccttgaactcgggtcattaa

SEQ ID NO: 165 - DNA, 1071 bp, Solanum lycopersicum
  source 1..1071: mol_type = genomic DNA
atggatgggaaggagaataatggtgttaatggttcaaacaaagttggtattcctaaatgttgcttgaaggctagggtatttgatcctgaacttgaagccaattgtcactctactgttgtttctggatggttctctcaacctcctacatcatttgatggtaaagagaaggtactgtacttcaacaatccaatgtggccaggagaagcacattccctgaaagtagaaaaggttttgttcaagggaaagtcagaattccaggaggttttggtttttgagtctgcatcctatggaaaagttctagtgctagatggcattgttcagttgactgagaaagatgaatgtgcctatcaggagatgatagcccatcttcctctttgttcaatcaaatctcctaagaaggttctggttgttgggggtggtgatggtggagttctaagggaaatttctcgacatagctctgtggagctgattgatatttgtgagattgataaaatggttatagatgtcagtaaaaagtattttccagatttggctattggctttgaggatcctcgtgtcaatcttcatgttggtgatgctgttgaatttctgaagaacacacctgaagggaagtatgatgctatcatagttgattcatcagatcctgtaggtcctgcaatagagcttgttgaaggaccattctttgcaaagatagccagggcattaaggcctggcggtgtactttgcaacatggcagagagcatgtggcttcatactcatcttattcaagatatgatctcaatttgccgtgaaacatttagttctgttcattatgcatgggccagtgttcctacatatcctagtggcgttattgggtttcttctgtgctcaactgagggaccatttgttgattttaagcaccctgttaatcctattgagaagttagagggcgctctccagcatcagcgggaactcaagttttacaattctgagatgcatgaagctgcttttgcattgccatgctttttaaggagggaggtgagcggtctccgcgatagcccacgttccgcagggagtggagtcaagatttaa

SEQ ID NO: 166 - DNA, 879 bp, Solanum lycopersicum
  source 1..879: mol_type = genomic DNA
atgggtgaagaaacgaaacaggagccaccaaaggaagaagtaaaaacagaggaagagcaaacagagccaaaaccaccttcgccttttgttttgtacgttgacttgcattgtgttggatgtgcaaagaaaattgagaggtccatttcaaagattagaggggttgagggagtagtaatagaaatggcgaaaaacacagtgacaataaagggagtaatagagccacaagcagtgtgcgacagaattacgaagaaaactaaaagagttgcaaaagtgttatcacccctgccggctgctgaaggtgaaccaattcctcaagtggttgcttcacaggtaagtggattgactactgtggagcttaatgtcaacatgcattgtgaggcttgcgccgagcaacttaaaaaaaagattctcagaatgaaaggtgttagaagtgcggaaacagaagccagcactggcaaagtaacggtgacgggcactatggacgctaacaaactcgtcgactacgtctatcgacgcaccaaaaaacaagccaaaattgtccctcagcccgaacctgagcccgaaaagcccgttgaagagaccaagcccgacgaggcgaagcccgttgaagaaccaacgcccgaagataagaaagatgaaggtacacccccgactgaaaataaagaagggggcgatggtgaaaaaccaccggagggagaaaaaaacgaaggcggtggtggaggagaagcgatgatcattccaattgaagatcattatgatgaacatacgattaataagttgatgtactatcatccttatcagccactttacgtcattgaaagaattccacctccgcagttattctcagatgaaaatcccaatgcatgttgcattacataa

SEQ ID NO: 167 - DNA, 1152 bp, Solanum lycopersicum
  source 1..1152: mol_type = genomic DNA
gtgaaaatcctcaacgaccgcatacttcggtatccggtcaccaaccagtttatatttaaagccttcatcaccaccaacaactccactactctgttctacaaaactctgcatcaactcaaatacacacaaaaaaaatcaatggctaaagttaagattggaattaatggattcggaagaattggccgattggttgctcgggttgctctccaaagaaatgatgttgaactcgtcgcagtgaacgatcccttcatttcagttgattacatgacatatatgtttaagtatgatagtgtacacggccagtggaagcatcatgagctcaaggttaaagatgagaagactcttctctttggtgagaaggctgttactgtttttgggtttaggaacccagaggaaattccatgggcacagactggagctgattacattgtggagtcgaccggtgttttcactgataaggacaaagctgcagctcatttgaagggtggcgccaagaaagttatcatttctgcccctagtaaggatgctcctatgtttgttgttggtgtcaatgagaaggaatacaagccagagctcaatatcgtttcaaatgctagctgtaccacaaattgccttgctcccttggccaaggttataaatgacagatttggaattgttgagggtctcatgaccacagtccactccatcacagccactcagaagaccgttgatggaccatcagccaaggattggagaggtggaagagctgcttcgttcaacattattcctagcagtactggagcagccaaggctgtggggaaagtgctaccagcattgaatggaaagttaactggaatggctttccgagtcccaactgttgatgtgtccgtggttgacctcacagtcaggttggaaaaagaagctacctatgatgaaatcaaagctgccatcaaggaggaatctgaaggaaaattgaagggaattctaggttacactgaagatgatgtggtttccacagactttgtgggagacaatagatcaagcatatttgatgccaaggctggaattgctttgagcaagaattttgtcaagcttgtagcatggtacgacaacgaatggggttacagtacacgagtggtggacttgattattcacatgtcatcagttcagtaa

SEQ ID NO: 168 - DNA, 960 bp, Solanum lycopersicum
  source 1..960: mol_type = genomic DNA
atggggtatgcaaatggtcttctgtgtccactagggaagcagccacttctctcttttggtgtcatctcagatgtccagtatgctgatattgatgacggcacctcattccttggtgttcctaggtattaccggcacagtgttagcgtgttgcaaagggcagttaagaaatggaatcaagaaaagcctaagtttgttcttaattttggggacattgtcgacgggtattgtcccaaagaccagtcaatgattgctgtcaagaaaattgttgatgaatttgacaaattcaatggtactgtctatcacatgattggaaatcactgcctttacaatcttcctcgaaaagacttgcttccattgctgagaatccctggccatgatgatcatgcctattttgatttttctccaattccggagtacagatttgtaatcctagatgcttatgatatcagtgccattggttggccggaagatcacccaaatactttgaaggccttgaaagttcttcaggagaaaaacccaaattcagacaaaaacagtccaagtggacttgtgggaccggcaagaaggtttctcatgttcaatggaggtgttgggaaagaacaattggaatggctggatcatgtccttcaggaagctaccaagttgaatcaaaatgtaatagtgtgttgccatttgcctttggatcctggagcttcatctcttgcagcattgttgtggaactatgatgaagtgatggatgtgattcatcgctatagctgtgtgaaggtctgtatgggtggacacgaccataaaggtggacaatcagtcgactcccatggtgtacatcatcgcgtccttgaagcagctcttgagtgccctcctggtactgattctttcggacatattgatgcttttgatgatagattattactttttggtactgatagaatgaagagcactgagatggtttttcgtcactag

SEQ ID NO: 169 - DNA, 1233 bp, Solanum lycopersicum
  source 1..1233: mol_type = genomic DNA
atgaatacttcttcttcaatcattttcaaatttgttgctttccttgttattttttcttcatttagtactcatgttttgtctactctccaagcaaatcaaacattcaggccaaaggaagaattgttgaaactcaagaaaataaagggttatctcaagaaaatcaacaagcgtcctgtcagaacgattaagagtccagatggagatttaatagattgtgttttatctcatcttcaaccagcttttgatcatcctcaacttaaaggaatgaagccattggaacctccaaagaggcctaaagctaatgaatcaatgaatataatggaagaaagtttacaactttggacagtatctggagaatcatgcccagaaggaactgttcctattaggagaacaacagagaaagatgtattgagaagtaattcattaagaagatttggtagaaaaattagaagaggtgttagacatgacacatcaagcaatgaccatgagcatgcagtagcatttgtgaatggagaacaatattatggagcaaaagcaagtattaatgtatggacaccaagagttacagatcaatatgaattcagtctgtcacaactatgggtcatttctggttcatttggtaatgatcttaataccattgaagctggttggcaggtcagcccggaattatacggagacaactatcctaggttctttacttattggacgacggatgcataccaagctacaggatgttacaacttattatgctcaggttttgttcaaataaataatagaattgctatgggagctgcaatatctccaagatcatcagttaatggtagacaatttgatattggcataatgatatggaaggatccaaagcatggacactggtggctagaattcgggtcgggtctattagttggatactggccagatttcttgtttagccatttaagaggacatgcaagtatggtacaatttggaggtgaagtagtgaattcaagatcaatggggtatcacacttcaacacaaatgggaagtggacattttgctgatgaaggatttggaaaagcttcttattttagaaatttgcaagttgttgactgggataataacttgattcctttgtcaaatcttcatctattagctgaccatccaaattgctatgatattagagctggaaaaaataatgtttggggtaattatttttactatggtggtcctggaagaagttcaaggtgtccataa

SEQ ID NO: 170 - DNA, 1092 bp, Solanum lycopersicum
  source 1..1092: mol_type = genomic DNA
atgaggaggattcatctatacatcgccattgcttatgttgtaatatttcaaactagttgttcagctcatttctttccaaatatttcatcaatcccttcttcattactcaaacctaatgccactgcttggacttcttttcaaaagttgttaggatgtcaacctggtcagaaagtcgatggcattgctaaaatcaaaaaatattttcaacattttggttacattaataatttgactagttttaatttcactgatgaatttgatgatactcttgaatctgctctcaagacgtatcagagaaatttcaacctcaaagccaccggtgtgctcgatgcgcccaccattcagcatctcataaaacccagatgtggaaatgccgatctagttaacggtactagtactatgaacgctggaaagccgcacacggtggctcactactccttctttcctggaagaccaaagtggcccgagggtaagactgatttgacttatgcctttctaccggcaaacaatttgacggatgatattaagagtgtcttctcacgtgcgtttgatcggtggtcggaggtaaccccgttgagcttcacggaaataccgtcatttcaatcggctgatatcaagatcggatttttaaccggagatcacaatgatggagagccgtttgatggtccgatggggacattagcgcacgcgttttcaccgccggcggggcattttcacttggacggcgaggaaaattgggtcgtcgacggtgtgccggtaaatgaagggaactttttttctatattgtcggctgtggatctcgaatcggttgcggttcatgaaatcgggcatttattgggtttgggtcattcatcggtagaagattcgattatgtatccgagtttagaatcgggtattcgaagagtggagctggtggaggatgatattaagggggttcaggaattatacgggtcaaacccgaattatactgggaccaatacaacattgactccgagcggcctggataatgatacgaatggagccccgattcgtagctcagtatggattcatgggtttttattggtggttggatttttcatttattcaatttaa

SEQ ID NO: 171 - DNA, 1071 bp, Solanum lycopersicum
  source 1..1071: mol_type = genomic DNA
atggctcatctttcagatcttgtcaatcttaatctctctgattcctctgagaaaatcattgctgaatacatatggattggtggatcaggaatggatgtaaggagcaaagccaggactctatctggtcctgttgatgatccttcaaagcttcccaaatggaattatgatggttctagcacaggtcaagctcctggagaagacagtgaagtgatcctatatcctcaagcaattttcaaggatccattcaggaggggcaacaatatcttggtcatctgtgattgttacaccccagctggtgaaccaattccaacaaacaagaggcacaatgctgctaaaatatttagcaaccctaatgttgttgttgaggaaccatggtatggtcttgagcaagaatacaccttgctacaaaaggaaattaattggcctcttggatggcctattggtggctttcctggaccacagggaccatactactgtggaattggatgcggaaaggcttttggacgcgatattgttgatgctcattacaaggcatgtatctatgccgggattaacattagtggtatcaacggagaagtgatgcctggacagtgggaatttcaagttggaccttcagttggcattgcatcaggtgacgagttgtgggcagctcgttacattctcgagaggattacagagattgctggagttgttgtgtcattcgaccctaaacctattccgggtgactggaatggtgcaggagcgcatacaaattacagtactaagtctatgagaaatgagggagggtatgaagttatcaaaaaggctattgagaagcttggacttaggcacaaggagcacattgcagcatatggtgaaggcaatgaacgtcgtctcactggaagacacgaaacagctgacatcaacacattcaaatggggtgttgcaaatcgtggtgcatctattcgtgtgggaagagacacggagaaggaaggaaagggatactttgaggacaggaggcctgcatcgaacatggatccatatgttgtgacctctatgatcgcggagactaccatcctgtggaacccttga

SEQ ID NO: 172 - DNA, 1797 bp, Solanum lycopersicum
  source 1..1797: mol_type = genomic DNA
atgtgcacaaaattcgtgaaggaaatggagaagatggcgttgtacgtcgtcgttttgctaatcatctgctatgcatcgccggcgagatcagacggttccgatcataagtataaatccggagatcaggttccactttatgctaacaaagtcggtccgttccataatccgagtgaaacataccgcttctttgaccttcctttttgtactccagatcatgtaaaagagaagaaggaagcccttggtgaggtcttgaatggtgaccgattggtcagtgccccttacaagcttgactttttgtatgataaggactcagaaatagtttgcaagaagaagttgtccaaggaagaagtttctcaattccgcagcgctgttgctaaagactactacttccaaatgtattacgatgacctgcccatttggggtttcttagggaaagttgataaggaaggaaagtctgaccccagcgagtacaaatactacttgtttaaacatcttcattttgagatcttttacaatgatgatcgtgtgattgagatcaatgcacgaactgatcctaatgccctggtagatattacagaggataaggaggttgatgttgacttcatgtatactgtaaaatggaaagagacaaaaacaccttttgataagaggatggagaagtactcacagtcttcctcgttgccgcatcacttggaaatccattggttctcaattatcaattcttgtgtgacagttcttctcttaactggttttcttgccacaattcttatgcgggtccttaagaatgactttgtcaagtatgcacatgatgaggaggcggctgatgaccaagaagaaactgggtggaaatacatccatggtgatgtgtttaggtacccaagtcacaaatccttgatggctgcagcacttgggagtggcacacagttattcacattgaccatctttattttccttctctcacttgttggtgttttctatccatacaaccgtggagctttattcacggcactggtcgtcatatatgctctcacatctggtattgcaggctatactgctgcctctttctattgccagctagaagggacaaactgggtcaggaatttgatattgaccggagccttgttttgtggacccctgtttctcacgttctgtttccttaacagcgtagcaattgcatacagtgctactgctgcactgccttttggaaccattgtggtcatctttctcatatgggctcttgtgacatcacctttacttgtattgggagggatagctggaaagaacagcagggctgagttccaagctccttgtcgaaccaccaaatacccaagagagatcccaccattaccttggtaccgcggaactctacctcagatggcaatggctgggtttttgcctttcagtgccatatatatcgaactttactatatatttgcaagtgtttggggtcacagaatttacaccatatacagcatcttatttatagtcttcatcattcttataattgtgactgcttttattaccgtggccttgacatactttcaacttgctgctgaagatcatgaatggtggtggagggctttcctttgtggtggatcgactggacttttcatctatggctactgcttgtattactattatgcccgttccgatatgtctggcttcatgcaaacctcattcttctttggatacatggcttgcatctgctatggtttcttcctcatgctggggactgtgggtttccgtgcagccctattttttgtccgtcatatataccggtcaatcaagtgcgagtag

SEQ ID NO: 173 - DNA, 894 bp, Solanum lycopersicum
  source 1..894: mol_type = genomic DNA
atggcggtggagagattcgcctgcggcgacgaactgccggaaggttgtatagctacggcactgtcgttgacgagccccaaggacgcctgccggctggcgctggtggcttcaactttccggtcagcagctgaatccgacgccgtttgggaacggtttctgccgccggattattatgatatcatttcccggtccattgacggtccagattcacttaatttcggttcaaagaaagacctttacttatacctatgtgataaccctattctcatcgacggtggtaccaggagcttctcattggagaaagggagtggaaagaaatgttacatgttagctgcaaggtcgttgcaaattgtttgggtcgatacgcctaggcattggagatggatttcccttcctgagtcgaggttttcagaagttgccgagcttcttgatgtctgctggtttgacatatctggcaaaataaacacaaatatgctctcccctgacactacatatggcgcttaccttgtgttcacgacaaaaaccaggacctgtggatttgaaaaccaacctgcagagagttcagtagggattgctgggcatgaaagatatacacatacattttatttgaaccctcagggaggaaggagacagtacagataccaagtagtcccgcgacggttaggaatattcacccaccacatggctcgcatattgagagaggaaacggaagatgcaccaaaagggtgtcgtcctaagcagagaagtgatgggtggatggaaatagagttgggagaattctttgtcaagaggggacaagaagctgaagtagagattagtttaacagaggtaaagggaggtgacaggaaagctggcttaattattgaagggattgatatcaggcctaaggagtaa

SEQ ID NO: 174 - DNA, 1080 bp, Solanum lycopersicum
  source 1..1080: mol_type = genomic DNA
atcattgtacaaattaactatctagctatagccatggtgtcaactactctatcaagttttctacttgttcttgttctttctttctttgtctcagaagctcaaaggcctgctttaactaaaggtctttcatggtcattttatcaatctagttgtcctcaacttgaatccattattagaaacaggcttcaaaaacaaatcaaggatgatgttggccaagctgctggcttacttcgtcttcatttccacgattgctttgttcaggggtgtgatggttcagtgttgctagatggatcagcaggagggccaagtgagcaaactgcaattccaaatttgaccctaagaaaaaggtcattcaagataattgatgatcttagaaaaaggatccaagatgaatgtggtcaagttgtgtcttgctctgatattgttgccattgctgctagggactctgttgtcttgacgggtgggcccaactacgatgtacccttaggaagaaaggacggagtcaactttgcaacggagcaagcgacaattgacaacctagtcgcaccctctgccaacaccacaaccgtcctctctctcctcgcaaccaaaggcctcgatgcgaccgatgcggttgcgttatctggggcccacactattggtatcagccactgtccttccttcaccgaccgtctctacccaaaccaagactccactatggacaagacatttgctaacaatcttaaaggtagttgtcccacggctgactcgaacaacacggtcaacatggacattcgtagccctaacgttttcgacaacaagtactacgttgatctcatgaatagacaagggctttttacgtccgatcaagatttgtatacggatcgaagaacacgagggattgtaacgagctttgcggtgaatcaatcgttgttttatgaaaaatttgttattggtatgataaaaatgggacaaatgaatgtgttgactggtggacaaggtgaaattaggaataggtgtgataggaggaataaggctaaaaaggttgatattgctactgttgttgaagaattggaggaaactttttctgctttgttttaa

SEQ ID NO: 175 - DNA, 3435 bp, Solanum lycopersicum
  source 1..3435: mol_type = genomic DNA
atgaagaggttatgtgctttcagctttgctcgtcgactcttcaaacctcgtacaacacattcccaggaatcaaaggagaaaatggagcaatcaccaattgtcaatgtcgaggacaactttgatttgcaggaatcaaaggagagcatggagaagtcaccaacggtcactgccaacaacaactttgatactcagagtgcaacagaaaatgagaaagagaatgggagttatgctgctactaacatcatcctaaaccatgaattctcggatgggctgcactcgtggcatcccaattgttgcgatgcttttgtggttccagcaggttctggataccataaaggattagcggcgaaggaaggctgttgttatgctgttgtctcaaatcgtaaggaatgttggcaaggcttagagcaagacattacaagcagggtatcagcaggttccacttatacagtttctgcttgtgttggggccactggtacttttcagggttctgttgaagtcctcgccacgttaaaactagtgtatcaaaattcagaaacaagttatctattcgttgcaaaaaaagctgcttcggaggagtgttgggagacattagaaggttcattttctctgtcaactatgcccgatcaagttatattctatctcgagggacctccagctggaactgacctgctcataaagtcagtagtgatatcatgtcctagttcaactgcttctgatagttctggcacatcatctgtatatattgatgatgataacatcataataaaccctcaatttgatgatggcctcaatagttggtctggaagaggctgcaaggtggccttgcatgactctatggcagatggcaagatcactccaatgtctggaaaatcttttgcatctgcaacagaacgcacccagagctggaatggaattcagcaagatgtgacaggaagagtgaagcgaaaactagcttacgaggtatctgctgttgtccgtatctatggtaacaatgtcactactgcagatcttcgcagtacattatatgttaaagcagctgacaaccgtgaacggtatataggcattgccagcgtccaagccacagataaagactgggtgaagttgcaagggaagtttcttataaatgactccccgtctcaggttgttgtctttttagaaggtccacctgcaggaacggatatcctccttaacaatttagtcataaagcatgcagctaaagctcctccttcttctccaccagttattgaggatgctggttttggtgttaatataatcacaaatacaagtttgaatgatggcacaaatggctggtttccacttggaaactgtacaatgagtgttcaaacaggctcaccacatattatgcctcctatggctagagatactcttggtgctcatgagcctttaagtggccgttatattctggtggctaatcgtactcaaaactggatgggtcctgctcagatgatcacggaaaaagtaaaactctatttgacataccaagtatctgcatgggttaaaattggacaaacatcaggacctcagaatgtaaatgttgcccttggagtagacagccaatgggtaaatggaggccaagctgagatcagcgatgatagatggcatgaaattggaggatcttttagaattgagaagcaagctgctaagattatggtttatattcagggtcctgttgctggtgtagacttaatggttgctgggcttcaaatttttccggttgacagacgtgaacggtttagacacttgaagaaacaaactgcaaagttacgcaagcgagacgtcatgttgaagttctctggatcagattcaggcaatttgtttggcacatttgttagagttaaacaactgcaaaacagctttcccttcggatcagccataagcagaacaaatatggacaatgaagacttcaatgccttctttgtcaagaatttcaattgggctgtgtttggaaatgagctcaaatggtactggacagaagcacagcaagggaatctcaactacaaagatgctgatgagctcctggacttttgcacaaaaaacaacatccaagttcgaggtcactgtatcttttgggaggtggtgggcacggttcaggcatgggtacaatccttgaacaaaaatgacttgatgacggctgttcaaaaccgtttaacagggctgctgacacggtataaggggaagttcccgcattatgatgtgaataatgagatgatgcatggttctttctatcaagatagattgggtaaagacatcagggtaaacatgtttaaaactgcacatcaattggacccttcccccatcctatttgtaaatgattaccatgttgaggatggctgtgacacccgatcttaccctgagaaatacattgagcacattcttgatctccaagagcacggtgcacctgttggaggtataggtattcagggacatattgacagtccagttggtcccattgtatgttctgctctggacaaacttggtactcttggacttccaatctggttcactgaggttgacgtgtcttcagataatgaatacgttagagctgatgatctagaagttatgcttcgagaagcttatgctcaccctgctgtagaaggtataatgttgtggggattctgggagctgttcatgagtcgaacaaatgcacatttagtggatgcagaaggagacatcaatgaagctggaaaaaggtaccttgctcttaagcatgagtggtcatctcatccgcatggtcatattgatgaacaagggcaattcagctttagtggattccatggctcctatgaagtggaagtcgtaacagtttcaaagaaaattactaagaaatttgtggttgacaagggcgataatgcactgagttcaaaggaaaatggaaacatcatccttaaccatgaattcttggatggtctgcacttgtggcgcccaaattgctgtcatgcttttgtggttccagcaggttctggataccgtaatggattgactatggctgttgtgacaaaaagtacgatatggtggcaaggcttagagcaagacattacaagcagagtatcagcaggttccagttgtactgcttctgatgaatcaaagcaggaaatggagaaggcaccgccaacaattactttgattctcagttcaaaggaagatgagaaagacactgggaacatcattttaaaccatgacttctcatatgggctttacttgtggaacccaaattgctgtgaggcatttgtggttccatcaggttaccataaaggattagcagctgctattgtaacaaatcgtagagaatgttggcatggcttggagcaagacattacaagcaaagtatcagaaggttctacttatacagtttctgcttgtgttggagcctctggtacttgtccgggttctgttgaactgacctgctcataa

SEQ ID NO: 176 - DNA, 651 bp, Solanum lycopersicum
  source 1..651: mol_type = genomic DNA
atgaagcggcaattcgacgccggagaaaccgattgggccaatttcaccggcggcgatcatgtgaagaagatgaagaaggagatgttagagttggaggaagagaggaagttaccgatgaagaggcaatttgaagctaactgtgcagctaaagtctccggcgatttgaaaaagatgaagcaggttatagagtcgccggatgcggatgcggatgcggatacggaggcggtgttgttgaatgagaatctactgtacgaagtactgaaacacgttgacgggcgcacgttagctacggcggcgtgcgttagcaaacagtggaatcaaacggccctagatgagcggctatgggagctgatctgcacaagacacagctgtaaccaacagcaacagctccgagccgtcgtcctcgctctcggcggcttccggcgtctctactcactctacctctggccactctcaaagccgtcgccgtctccatcggcgtcttcttcgaatccaccggcttccgcctggccttgccttcctccggcgcctactcggccgttgaaatacgctgctgcaaacaccagatggggaaaagacgaagttaatctctcgctatcgctgctatcaattcggtactacgagaagatgaatttcaaagctaataaataa

SEQ ID NO: 177 - DNA, 1458 bp, Solanum lycopersicum
  source 1..1458: mol_type = genomic DNA
ccatcttctttttccatacctcaactaaaacaatcaaacatggcgattcatctctccgctctaacaatctcgatcctcgtaatggtcgtgtcctccgccgccgtaacatcggcggcggcagaggacatgtcgattataagctacaacgaaaaacatcacacgatcggcgccggtcggaccgatgacgaggtcatgtctatgtacgaatcgtggttagttgaacacaaaaaagtgtacaacgctttgggagaaaaagataaacggttccagatctttaaggacaaccttaaatacatcgatgaacataacgctatgccggagaaaagttataagctcggtttgactaagtttgctgatctgaccaacgaggagtataggtcggtttatttgggtacgaagccggatgctagtcgcaggttgtcgagtagacaaagtgatcggtatgctccgaaggtcggagataggttgccggaatctgttgattgggtgaagaaaggtgtgcttgttggtgttaaggatcaaggacaatgtgggagttgctgggctttctcagcagttgctgccattgaagcagtaaacaaaataaagactggagactcgatctctttatccgagcaggagctagtagattgtgatacttcttctaacaatggttgtgacggtggtctcatggactatgcttttgaattcgttatcaaaaatggaggactagacactgaggaagattacccatatactggcgaggatggaagatgtgacctaacaaggaaaaatgccaaggttgtcaccattgacgggtatgaagatgttcctgctaatgatgaaaacgcaatgaaaaaggctattgcaagtcagcctgttagcgttgctattgaagctggtggcaaagacttccagcactataaatcgggcatctttactgggaagtgtggcgcagcagtggaccatggtgtggttgcagtgggatatggtagcgaaaatggcatggattattggattgtgaggaactcatggggtgcttcctggggagaacacggctacctcaggatgcagcgtaacattgctaacccaaagggtttgtgtggtattgctacagttgtttcttaccctgtcaagacaggccaaaatcccccgaaaccagctccatctcctccatcgccagtcaagccacctactacttgtgatgatatgtacagttgcccatcaggaacaacgtgttgctgtgtctacgagtactatcacatgtgctttgcttggggttgctgccctatggaaggagctacttgctgtaaagaccataacagttgctgcccacatgattatcctgtctgcaatgttaaagccggcacctgctcaatcagcgagaataaccctctttcagtcaaagcaatgtcacacattcttgccaaacctattggatccttcagcaatcagggaatgaaaaacactatctcttga

SEQ ID NO: 178 - DNA, 1104 bp, Solanum lycopersicum
  source 1..1104: mol_type = genomic DNA
atattgaaaattgaaaacaacaccacccaatttttctctctcttcttcttttcccccctttcactttctctctctaaaaacctagctatggcgcttcctcatcaccatctccaactacacattcaacaacaaccacatcaacaacagcaacagtccaaatcttacagagatttgtacaacaatatggatggtcagataacaaacccagttgtttatttcaatggctctaatcttcctgaacagtctcaacaccctccttatatacctccctttcaagttgtggggttggctcctggtacagctgatgatggagggttggatttgcagtggaattatgggttggagcctaagaagaagaggcctaaagagcaagattttatggagaataacaactctcaaatatcttctgttgatctgttgcagcggcgatcggtgtccactggcttggggttgtctttggataatggacgtttggcttcgtcgtgtgactcagcgtttctagggctcgtgggtgatgatattgagcgagaattgcagaggcaagatgctgagattgatagatatatcaaagttcagggtgatcgcttaaggcaagctgtattggagaaggttcaagctaatcagattcaggctatcacctatgttgaagagaaggtccttcagaaacttcgagaaagagacacagaggttgatgatatcaacaagaaaaacatggaacttgagttgcggatggaacaattggctttggaagccaatgcttggcaacagcgtgccaaatacaatgagaacttgattaacacacttaaggtaaatctacaacatgtttatgcacagagcagagatagtaaagaaggttgtggtgacagtgaggtagatgacacagcatcatgctgtaatggacgggctacagatctccacctgctttgccgggatagcaaggaaatgaaggagttgatgacgtgtagggtctgtagaaccaatgaagtttgcatgctattgttaccatgtaagcatctctgcctgtgtaaagagtgtgagagtaagcttagtctttgtcccttgtgtcagtccactaagtatataggtatggaagtttacatgtag

SEQ ID NO: 179 - DNA, 639 bp, Solanum lycopersicum
  source 1..639: mol_type = genomic DNA
atgaaggcttcattcaaagcacgatatgagcccgacaaagccgccgccaacgctactgttgcattcaacgccggcgaccttaagctccgagcctccatgaccgatgctactgttgtcaaaggtcccagcttaaacggattagctctggccgttgagaaacctggattgttcatcgtcgactacaacgtcccgaagaaggacattaggtttcaatttatgaactcaatcaaggttctggagaagccattgaatttgaattacattcattttcatggagacaagcgaacaatattggatggaactttggtggttgattctgccaacaaggtgtcggctaatcacgtcatgggctcaggaagttgtaaattaaagtacacatatgtccacggaggaatcactactttcgagcccatctatgatacagcaaagaatgcgtgggacttcatggtatcacgtaaagtttatggggatgacgtgtttaaggctacttatcagacaacaagcaaaaatttggggctcgaatggtccaggagttctaagttaaatggatcatttaagatttgtgcatctctctgtttgcttgaggaacgcaaaatcccaaagttaagtgctgagacgtcgtgggactttgaaatgtga

SEQ ID NO: 180 - DNA, 975 bp, Solanum lycopersicum
  source 1..975: mol_type = genomic DNA
atgttaaacgccgagcatcacttttcactttttaaggctaagtttggaaagatctatgcttctcaggaggaacatgaccatagattgaaggtattcaaggctaatcttcaccgtgcgaagcgtcaccagctccttgacccttccgctgagcacggtattacgcagttctccgatttgactccgtcggagtttcgccggacttaccttggacttaacaagccccgacctaatcttaatgccgagaaagctccaatcctcccgactaaagatcttccatctgattttgactggcgtgaaaaaggagccgtcaccgatgttaaaaatcagggctcatgtggatcatgctggtcattcagtacgactggagcagtggagggagctcactttttggcgaccggagagcttgtgagccttagtgaacagcagcttgtggattgtgaccatgagtgtgacccggtggaaaaaaatgattgtgatgcaggttgcaacggtggtcttatgactactgccttcgagtatacccttaaggctggaggtctccaactggagaaagactatccttacactggcaggaatggcaaatgccactttgacaagagcagaattgctgcctctgtatccaacttcagtgttgttggtcttgatgaagaccaaattgctgctaacctgctgaaacatggtcctcttgcagtggggatcaatgctgcttggatgcagacatatgttagaggagtttcatgcccattaatttgcttcaagcgtcaggatcatggtgtcctcctagtcggttatggatctgaaggttttgcacctatacgtctcaagaataagccttactggatcatcaagaattcttggggaaagacttggggagaacacggatattacaagatctgcaggggtcacaatatctgcggagttgatgccatggtctctactgtgactgccacacatactactaatcctaatctttaa

SEQ ID NO: 181 - DNA, 1296 bp, Solanum lycopersicum
  source 1..1296: mol_type = genomic DNA
atggccggccaaacagtcattgtttccggtttgaatccggcggccattcttcagtccacaatcggtggagctccggtggcagcggcggcggagaacggccatacgagaaaagttgttcctctgtcaaaagatgcccttcaggatttcatggtttcaatcataacacagaaattacaggacgataaacaacccttttatgttctagatttgggtgaagttgtttcccttatggaacaatggaactctgctttaccaaatatccgtccgttttatgctgttaaatgtaaccctgaaccttcatttctctccatgttgtctgctatgggttccaattttgattgtgctagccgtgctgaaattgagtatgttttgtctcatggtatctcgccggaccggattgttttcgctaacccttgtaagccggaatccgatattatcttcgccgagaaaattggggttaatctcactacttacgattccgaggatgaagtttacaagatccgtaaacatcacccaaaatgcgagcttttgcttcgaatcaaacccatgaccgacggcaacgcgagatgcccaatgggtccaaaatacggcgcattaccggaggaaatcgagccgctcctccgtacagctcaagccgctagactcaccgtctccggcgtgtccttccatatcggcagcggagacgccgattccaacgcttatctaggagccatagccgcagcgaaacaggttttcgaaacagcagctcaactcggaatgccaaaaatgacggtactcgacatcggcggcggatttacctccggccaccaattcaccaccgctgctcccgccgttaaatcagctctggaaacccacttccacgacttccctgagctaaccatcatcgctgaaccaggtcgtttcttcgccgaaacagcgttcactttagcaacaaccatcatcggcaaaagagtcagaggtgaattaaaagaatactggatcaacgatgggttatacggatcgatgaactgtgtactttacgaccatgcgacggtgacggcgacgccattagcgtgcatgtcgaatcgtaataacctcaactgcggcgggtcgaaaacgtttccgtcgactgtattcgggcccacctgtgatgcacttgataccgtgttaagggattaccagttaccggagctacaggttaatgactggctgatttttcctaatatgggtgcttatacaaaagctgctggatcaaatttcaatggatttaacacatccgccattgttactcaccttgcttatgcttatccaaactaa

SEQ ID NO: 182 - DNA, 1218 bp, Solanum lycopersicum
  source 1..1218: mol_type = genomic DNA
aacatggctaaaatagcctatcttcttaccactcttttagtggctttagtgtcacttagcttcccttcagaatgcaaagcaaattactactacacatctccacctccaccaactccagtttataagtataagtctccgccaccaccagtttataagtacaagtcaccaccaccacctcctccaatctataaatccccaccgccacccatttataagtataaatctccaccaccacctccagtttacaagtacaaatctccaccaccacccgtctacaagtacaagtctcctccaccaccacctccagtttacaagtacaaatctccaccaccacctgtctacaagtacaagtctcctcctcctccacctccagtttataagtacaaatctccaccaccacccatctacaagtacaagtctcccccaccacccgtttacaagtacaagtctccaccaccacctgtctataagtacaagtctccacctccacctgtctacaagtacaagtctccaccaccaccagtatacaagtacaagtctccaccacctccagtatacaagtacaagtctccaccacctccatctataagtacaagtctccacctccacccgtttacaaatacaagtctccaccacctccagtctataagtacaagtctccacctccacccgtttacaaatacaagtctccaccacctccattctacaagtacaagtctccacctccacccgtttacaaatacaagtctccaccacctccagtctataagtacaagtctccaccacctccagtctataagtacaagtctccaccacctccagtctacaagtacaaatctccaccaccacccgtttacaaatacaagtctccaccacctccggtctacaagtacaaatctccaccaccacccgtctacaagtacaagtccccaccacctccagtctacaagtacaagtctccaccaccacccgtctataagtacaagtctccaccacctccagtctacaagtacaagtctccaccaccacctatttacaagtacaaatctcctccaccaccagtttacaaatacaagtcaccaccaccacccatttacaaatacaaatcacccccacctcccgttcacaagtctccagccccttactattacacttctccacctcctcctagccactactagtttcacaatctcgaacgaggtcaagaggaataattaa

SEQ ID NO: 183 - DNA, 651 bp, Solanum lycopersicum
  source 1..651: mol_type = genomic DNA
atgggatcggaggggccaaaatctgtgaccattcatgtcacggggttcaagaaatttcacggtgtagctcagaatcctacggagacggcagttagcaacttgaaatattatgttgaaaagaaaggattacctgctggtgtgaccttgggtagctgtactgttcttgaaacagctggagagggtggacttccaacattgttgaaggttatagaggcgtcctctgctagtaacagttcaaataatggacaagtcatatggcttcaccttggggtaaacagtggagcaacaaagtttgcaattgagaggctggcagttaatgaagccacatttcgttgtgcagatgagcttggttggcaacctcagcgagtgcctattgttccagaagatggaggaataaatcaaataagaaagacatgttgccctaccgagtcaatcctggagttgttaaagaagaaagggtttgatgtgacgttgtcggacgatgcaggtcgttttgtgtgcaattatgtgtactatcattctctccgatttgcagaggaaaaaggtcacaagtgtctgtttgtccatgttcctaccttccatagagttcatcaagagaaacaaatggaatttgttgctgctctgctggaggctattgcatcaacttgttga

SEQ ID NO: 184 - DNA, 372 bp, Solanum lycopersicum
  source 1..372: mol_type = genomic DNA
atggaccctaacgacctagcgaaggcatttgtggagcactactactccaccttcgatagcaacagggctgcgcttggaaatctgtaccaagaaggttccatgttgacatttgaaggcattaaaactcaaggttcacaaaatattgtcgctaaacttactagccttccctttcagcagtgtcatcataccatcactaccattgattgtcagcattctggtcctgctggtggcatgattgttgtcgtctccggtaatctccagctagccggcgaacagcatgctcttaagtttggccagatgtttcatctgatgccaacgccacaaggaagcttctacgtcttcaatgacattttccggttgaattatggctga

SEQ ID NO: 185 - DNA, 768 bp, Solanum lycopersicum
  source 1..768: mol_type = genomic DNA
atggcggcgaatcaaaatgtaattgtaatgagacacggtgaccggttggataactttgaggaattgtgggtgatgaaagcggaaagaccgtgggatccacctttgcatcaggatggtaagatccgggcattttgtacgggtcaaaagatccgatccagtattgatttcccgatccatcgggttttcgtttctccgtttctccgatgtgtgcagactgctgcagaggtcgtacgtgctctttgtgacgttgctgatcacaacggcgagtctaatgtcttgtcatccaattccgattcagtcattattgatccatctaaagtcaaagtatctattgaatatggtttgtgtgaaatgctgaatttagtagctgtaagagcttctgcagctcctaaggatggggatttcaaattcagcatctcacaatatgaatctgagttgcctgctggaactgtagataacactgttgaacctgtgtataagaagctgccagagtgggaagagtcgttggaaagtgcaagggctagatatgtgaaggtagttaaggcccttgctgataagtatccttctgagaacctgttgttagtcacacatggtgaaggagtaggttccatctttactgagcttaacaaggattcaactgtattagaggtagcgtattgtggacatctgcacgtaaagagaagtatccaatccggagagaaacaaccatttactgctggagagtttgtatacgagaaacaaacgggtataatatctgctgctaaataa

SEQ ID NO: 186 - DNA, 1635 bp, Solanum lycopersicum
  source 1..1635: mol_type = genomic DNA
atggagtcggagaaaccccaaaatccatctccaatggaaccacaagaagaaggcagcaacaaaatctccaaaaaagaagccgccaagctggagcgtcagcgccgccgtcaagaggcagccgccgccgccgccgccactgctgtctccactgtctccattgaagaatctgatccactttccggaaactacggtgacgtagagctgaacgatctgcaatctaaggccgtgacgggtcgaaagtggactgaagttgggtccatcgcgccagaaatgaaggaccgggtggtcttggttcgggggagggtacagacgattcgtcctgttgggaaaaagatagcttttgtggttgtgagggaacgaggatttacggtgcagtgtgtattgactgtgaagcctgaattggttagttcacaaatggtgaaatatgctactagtttgagcaaagagtccattattgatgttgaaggtgttgttacaataccagagaagccaattactggtgccactcaacaacaggtggaagttcaaataagcaaactttattgtgtcaacagggctgtgcccactctaccgattaatattgaggatgctgctagaagcgaggtggagatagaacaagctttagagaaaggagagcagcttgttcgtgtaaatcaggatactcgcttaaacttcagaatactagacatgcgcacacctgcaaatcaaggaatcttccgcattcaatgtcaaatcgaaaatatattcaggcagtttttgctctctgaaggatttgtaggaatccatactccaaaattgattggagggtctagtgaaggaggttcagctgttttccgattagattataaaggtcaacctgcatgtctggcgcagtcacctcaacttcacaaacaaatggcaatatgtggtgattttggccgtgtatttgaagtaggccccgtctttagggcagaagattcttttacacatagacatctgtgcgagtttacaggtcttgatgttgaaatggagatcaaggaacattattcagaggtcatggatattgttgaccgcttatttgtggccatgtttgatagcttgaatgagaaatgcaaaaaggaacttgaagccgttggaaagcaatacccttttgaacctctgaagtatttgcgaaagaccttacgacttacatttgctgaggggattcaaatgctaaaggaagctggtgttgaagttgatcctcttggggacctaaatacagaatcagaaagaaagcttgggcagcttgttgcggagaagtatggaactgaattctatatacttcacaagtacccacaggctgttcgtccattctataccatgccttgtcatgacaatccatcttacagcaattcttttgatgtctttatcagaggggaggaaattatctcaggagctcagcgtgtccatgttcctgaactcttggaaaaacgtgccggagaatgtggaattgatgtgaagacaatatcaacatacattgattctttccggtatggtgcacctccccatggtggatttggagtcggattggagcgtgtggtgatgcttttctgtgctctcaataacattcgcaagacatcacttttccctcgcgacccacaaaggattgtcccatga

SEQ ID NO: 187 - DNA, 2415 bp, Solanum lycopersicum
  source 1..2415: mol_type = genomic DNA
atgagtggtactccgaacaaaagacctcacgaggatggtggaaatggtgggagtagtaaccatagttactcttctgctccaaaatactcacatgatgactctggtgcatttcccaaggtgatgagctcaggaacacctgaatatcatgcctcctttgatgtgggccagaatgctcggatgccgaagattcaacggactgaatcttcacgagatgcagatagaagatctcctgtgcttccaatgtaccgtgtctcatcatgtccagttgtttcacatcctgatcattctgttgcttcagaaaataggttggagcccaaggaagttaacaaggacgtcaaggttgagaatcgtgatgccaaaagtgaaataagggagttgtaccaagggactaaatctgacaaggatgatagatttgagaacagagctgatgatggtaaggacattaaaaatagtagggatacttaccctgaatacaagggagatgtgaagacagataaggacaggtttagcggagtgagttggaaagatccgaaagaacagaccaggggaaaaagatatcctgatctccctgttcctgtcgggaacatggatccatggcatgcgtcaagaacccatggtgctgctgagataggaaaagaagtctcaaattctgagaacagggattttgctaaagtgcgtgaagccgttgctgaaaataagatggatttgaaaggtgacgataaatacaaagataaagagagaaaaaggaaagaagggaagcaccgggaatggggagaaagggataaagagagaaatgattgtcggaacaatttacaactagggaatagcacttctgataacaaggaattgcttaaagaggaaagggaatctgagcggtgggagaaggaaagaaatgatctttcgaaggataaggacagaccaaaggactgggaaaaggaccatgcaaagagggaagtgtggaatggagtggagagggaggttttgcagagtgagaaagaagtgattgatgttcctggaaaaacaaacgagccggaaaactcaacagtggagcagaagaaacagaaagatcatgataactggaaaaatactgacagggatggaagtgagaggagaaaggaaagagatactgatttggaaggagagaggcctgagaaacgtgtcaggtgtcatgataaagaaccagaggaaggggacctggatactgaaggaggaggagaaagggaaagagaagcttttaattatggagttcagcagcgcaagagaatgtcgcggccaagagggagccccatggccaatcgcgatcctcgttttaggtcgcacactcatgaaaatgaaggatctcaagtgaagcatgatgtatctgctgtcaattacagagttggtgagtgtatgccagaactgattaaattatggaaggaatatgaatcatccaaagcagatgaagcatctgatagctctccaagtgatcctactctagaaattaggattccagctgaacacgtatcagctacaaatcggcaggtgagaggtggccaactatggggaacagatatatacaccaatgactcggatcttgtcgcagttcttatgcacacaggttactgtcgtacaactgcgtctcctcttttgcctactattacggagttacgtgctactatcagggtactacctccacaaaattgctacatatctactctgaggaacaatgtgcgatcacgtgcgtggggagctgcagttggctgcagctatcgtattgagcggtgctctgttgtgaagaaaggaggtggaacaatcgatcttgaaccttgtctaacacattcctcaaccttggagcctactcttgctccggtggcggtagagcgcactatgaccactcgagctgcagcttcgaatgcactacgacaacagaggtttgtacgtgaagtgacaattcagttcaacttatgcaatgagccttggctcaaatacagtatcagtgttgttgctgacaagggtctaaaaaaggccctttttacatcttcacgcctgaagaagggagaagttctttacttggaaactcattctaagaggtatgagctctgttttagtggtgaaaagatggttaaggctacaacttctctgatgcatgaaatggatgttgacaaacctcaaagtcacaatttacacatggcaaacggagaaaaaaatggagtgaatggtgagaatacgatggtagatatgttccgactgtctcgttgtaagaagcccctgcctcagaaactaatgcaatcagttggaattcctttgccccttgaacatgttgaggttttggaggagaatctggagtgggaaaacattcaatggtcacaaactggtgtttggattgctggaaaagaatatcctcttactagagcgcattttctttccccaaattag

SEQ ID NO: 188 - DNA, 855 bp, Solanum lycopersicum
  source 1..855: mol_type = genomic DNA
ttcttcacttcaaattactcaaccaaaacgatgtcgtttgatgaagatgaagaatccttcgagcacacgctcctagtagtgcgtgaagttaacgtcttcaaaattcctcctcggccaacctccggcggctacaaatgcggcgagtggcttcaaaccgacaagatctggtcgggtaggctccgggtcgtgtcatgtaaggatcggtgcgagatccgattagaggatcccaattccggcgagttgttcgcagcttgtttcgtgccgccgggacagagggagaattcggtggaatcggtgcttgattcgtcgaggtactttgtgttgaagatcgaagatggtacagggaagcatgcgtttattgggttagggtttagcgagaggaatgaagcgtttgatttcaatgtagcgttatcggatcatgagaagtatgtgaagagagatattgagaaagatggtgatgaagctgagacgagtagtgatggtcatattgatattcatcctgctgttaaccatcgattaaaggaaggtgaaaccatcaggataaacgtgaagaacaaatcatctagtggaatgggcatgctttctgctgctgttcaatcaaaggccattaccattgctccaccccctagcggagctaacaagatcaggtctcctcttccaccccctccgaatgatccagctatttctcggaagacttctaccactgctagtattgcactcaaggggccaaatggaagctcaatgcagtccagtgatccaatttccgatctctctcaacttgagaggagccttccttcagccactggatctggttcgagcaagaagaccacagcagctggatgggcagcattttga

SEQ ID NO: 189 - DNA, 870 bp, Solanum lycopersicum
  source 1..870: mol_type = genomic DNA
atgggggatcaaatagcaagaggtgaggatttcgagagcaaggctgagaagaaactcggcggatgggccattttcggttccaaatacgaagacgcagctgatctgttcgataaagctggcaattgctttaaactcgccaaatcatgggataaagctggagcagtatatgttaaattggctaattgtcatcttaagttggatagcaaacatgaggctgcaaatgcttatgcagatgctgctcattgctacaagaagtcaaatattaaagaggcagtatcatgtttagagcaggcagtgaacttatttctggacattgggaggctaaatatgtctgcaagatattacaaggaaattgctgaattatatgagcaagagcaaaatttggatcaggctattaattactatgagaaggcagctgatcttttccagagtgaagatgtaactacaactgcaaatcaatgcaaacagaaagttgcacaattctctgctcaaatcgagaaatatccgagagcgatcgagatctttgaggagattgcacgtcactcagtcaataacaacttgttgaagtatggagttagaggacatcttcttaatgctggtatttgccaactttgtaaaggtgatgttgttgcaatcaacaatgcgttggagcgatatcaggagctggatccaacattttcagggacacgtgaatgcaaattgctagtggatttggcagctgcaattgatgaggaagatgttgcaaagttcactgatgctgtcaaagagtacgatagcatgacacaactggatgcttggaggacaacactacttctaagagtcaaggaaacattgaaggcgaaggagcttgaggaagatgatcttacttaa

SEQ ID NO: 190 - DNA, 1023 bp, Solanum lycopersicum
  source 1..1023: mol_type = genomic DNA
atggaggtcataatgaacaatcacaacaatggcaccatcaacaccaacaccaaaattattcacaaaaatggtagcatttgcaatggcaatggcaatgttaatggcaactcccacacccataacaatgaaaataagcttgtagagttcactaactctatcaaaccgggttggttttctgagttcagcgcactctggccaggtgaagcattttcacttaaaattgaaaaattactattccaaggaaagtctgattatcaagatgttatgctctttgagtcagcaacgtatgggaaggtattaacattggatggggcaattcaacacacagagaatggtggatttccctacactgagatgattgttcatctaccacttggttccattccatcccctaaaaaggtgttaatcatcggtggagggattggtttcacgttgtttgaggtctctcgctatccaagtattgaaaaaatagacatagttgagatcgatgacgtggttgtagacgtatctagaaagtttttcccatacctagcagcaggatttgatgatccaagagtcagccttattattggcgatggagctgcatttgtgaaagctgctcaacctggatactatgatgcaattattgttgactcttctgatcctattggtccagcaaaagacttgtttgaaaggccatttttcgaagcagtggcaaaagccctaaggccaggaggagtagtgtgtacacaggcagaaagtatttggcttcacatgcatctaattaaaaaaattattgcaaattgtcgacaggtctttaagggttctgtcaattatgcatggactaccgttcctacttaccctactggtgtgattggttacatgctttgctctacggagggaccagaagttaatttcaagaatccagtgaactctattgacaaatatgtcaaatccaagggacctttgaagttctataactctgatattcataaatcagcttttattttgccgtcttttgcaagggatttgattgaatcttga

SEQ ID NO: 191 - DNA, 840 bp, Solanum lycopersicum
  source 1..840: mol_type = genomic DNA
atgggacgttcaccttgttgtgaaaaagcacatacaaataaaggagcatggactaaagaagaagaccaacgcctcatcaattatatacgtgctcatggggaaggttgctggcgttctcttcctaaagctgcaggattgtcaagatgtggaaagagttgcagattgagatggataaattatttaaggcctgatctcaaaagagggaattttacagaagaagaagatgaattgataatcaaacttcatagtttgcttggaaacaaatggtcagttatagccggaagattacctggaagaacggataacgaaatcaagaactattggaatacacatattaaacgaaaactcattagtcgtggcattgatcctcaaactcaccgtccactcaacaacaacgccactaactcccacaccaccaccaatatcaccaccgcagtcacaaccaaaaacatcaacttggatttcacaaacgttgaccaaaaacaacccaatattatgattgccacgtcatcgtcatatgatgaaacaaaatgcaacagtggtacaactgaggaaacaaagccactagaaattattattccaaaaataccctcacaagttatgattaatcttgaactttcaattgggttgccgttacatactgatcatatttcttcaccagagtcaacggcctcatacaacttcttgaccaccgtagcaccgccgccaactgcggcggtaccggcggcggagatgatggcgaagactgtttgtttgtgttggcaaattggatatcatggtggtggcggtcagtggtgtggtaaatgcaaaaacacaaatggattttacagatattgctga

SEQ ID NO: 192 - DNA, 891 bp, Solanum lycopersicum
  source 1..891: mol_type = genomic DNA
atggcgacgcaagatgtaaggactctttcgactaaagaagctgacattcagatgatgttggctgctgaagttcacctcggtactaagaattgtgacttccaaatggagcgatacgctttcaagcgccgtaacgatggtatctacatcatcaaccttggaaagacatgggaaaagcttcaaatggctgccagggtgattgttgctattgagaatcctcaggacatcattgttcaatctgccaggccttatggacagagagctgtcttgaaatttgctcaatatactggtgcacatgctattgctggacgtcacacccctggaacttttaccaatcagcttcagacctcatacagtgagccacgactcttgattctgactgatccaagaactgaccatcagccaatcaaggaagctgcacttggaaacatcccaactattgctttctgtgacactgattctccaatgcgttatgttgacattggtatccctgccaataacaaaggaaagcacagcattggtgttcttttctggatcttagcaaggatggtactccagatgcgtggggccattaatcaaggacctaaatgggatgttatggttgatctcttcttttacagagagcctgaagaggcaaaggagcaagaagaggaagtgccagcaattgcagattatgcagactactctgcgagtgctgcccttggtggtgactggactagtagccaaatccctgaggcgcaatggactgcagatgctgcagcacctgcagtaggtggtggttgggctggagatggagcagctgatggcggatgggatgcagcagcagcccctgcaccagttcctctaccagtaccagatgttgcccctacctctggtgctacaggctgggaatga

SEQ ID NO: 193 - DNA, 1104 bp, Solanum lycopersicum
  source 1..1104: mol_type = genomic DNA
atggactctctgaattcttacgcatcgtcggcggcgatggcgcagcaaacctgggagttagagaacaacatcgtaacaatcgacgcgccgtcggggtcgaaaccggaaaactccgcgtcggacgctatattccactacgacgatgcggcacagacgaagtttcagcgggagaagccgtgggcgagtgaccctcactacttcaagcgtgtgaagatctctgctcttgctcttctcaagatggttgttcacgcgcgttccggaggtacaattgaggtcatgggactcatgcagggtaagactgacggcgatgccatcattgtaatggacgcttttgccctccccgttgaaggcactgaaactagggttaatgctcaagctgatgcgtatgaatatatggtcgaatattcacagaccaacaagcaggctggtcgtttggaaaatgtggtcgggtggtaccattcccatcctggttatggatgctggctctctggcattgatgtatctacccaaatgctcaaccagcaatatcaggaaccctttcttgcagttgttattgatccaacaagaactgtttctgctggaaaagttgagattggtgcctttcgaacatatcctgaaggatataaacctccagatgaccccatctcggagtaccagaccattcctttgaacaaaatcgaagactttggagtacattgcaagcagtattattcattggatattacatattttaagtcttctctcgattgtcgtctcttggacctactatggaacaagtattgggtgaacacactttcatcatctcctttgcttggaaatggagactatgttgctggacagatatctgatcttgctgaaaagttggagcatgctgaaaatcagctgtctaattcacgttatgcatccttaatggcaccccctcaaaggaagaaggaggaagaatctcagcttgctaagattacacgtgatagtgctaagattactgtcgaacaagttcatggcttaatgtcgcaggtaattaaagacattcttttcaatagtgttggtaagtcaagcaagtcacagacggaatcctcaggtcccgagcccatgatcgaaagctga

SEQ ID NO: 194 - DNA, 897 bp, Solanum lycopersicum
  source 1..897: mol_type = genomic DNA
atggcatccatgattgaatctgcttggacgtatctgattacaaatttcaatgacttccaattggcttgtcttggaagtttctttcttcatgaaagtgtcttcttcttgtctggacttcctttcattcttctagaaagggcaggatggttgagcaaatataaaattcagacaaaaaataatactcctgcagctcaggagaaatgtatcactcgcctgttgctgtatcatttttgtgttaatctcccgcttatgatggcatcctatccagtcttcaaattaatggggatgcgaaatactcttccgttgccgtcctggaaagtgatctctacgcaagtattattctactttatcctggaggattttgtattctattggggacacaggattttacatactaaatggctctacaagcatgtccacagtgtccatcacgagtatgcaacaccatttggattgacctctgaatatgctcaccctgctgaaattctttttcttggatttgctacaatagtgggtccaatcattactgggccccatttgataacattatggttgtggatgatagttagagtccttgagacagttgaagcacattgtggttaccatttcccatggagcctatcaaacttcttgccattatatgggggggctgattttcatgattatcatcaccgactgctctacacaaagtctggaaattattcttcaacatttgtttacatggactggttatttggtactgacaagggttacagaagattgaaggtgctgaagagtgatgcatgtgaagctgagggtaaagaaatgtagagatgtgaaagggtaagttttggctggtctggcattgcaagacgggcttatgtacaagagcgtataggtgaaggtttctgttgttga

SEQ ID NO: 195 - DNA, 603 bp, Solanum lycopersicum
  source 1..603: mol_type = genomic DNA
atggaaaccactgctacgccggaggagtggccgccggacgggcaatttccatttgatctttttgtatcgaagaagtataagtccttctgggatgctggaaacatccaatttaccgatgctgttggtaatttgttcttcagcgttgaccgtcgtcaacctcagaattcggcagcccagacccaccctcaaaaactaattctcgatgcatcggacaataccctgattcgcttagttcgattaactaaaggatcatggcaaggctttatggtaagtgataatgaagaaaaggcgttgatgttcagtgtgaataagacactaaatacattcactaaactagagtttgacatattccttggtgatggacatgttgaaggcgcagaggctgatcttaagatgaaaggttctgccttcaagagatcatgcaccatctacaaaggcaattccatagtagctgagactagccttatgtacacacttgggttcaggaagcatttcattccaaggaacagatttcgagtaaccatatttcccggtttgactgagcttagccttgttgtggctttggttgtagtattttttgacaaacagaaattttggctataa

SEQ ID NO: 196 - DNA, 660 bp, Solanum lycopersicum
  source 1..660: mol_type = genomic DNA
atggggagaagacctgcaagatgttatcgtcagattaagaacaagccttacccaaaatcaaggttttgccgtggtgtccctgatccaaagatcaggatttatgatgtgggcatgaagaaaaagggagttgatgagttccctttctgtgtgcatttggtcagttgggagaaggagaatgtctcaagtgaggcacttgaagctgcccgtattgcttgcaacaagtacatgaccaagtctgccgggaaagatgcttttcacctaagggtcagggttcatccattccatgttttgcgaattaacaagatgttgtcgtgtgctggagctgatagactccaaactggtatgaggggagcttttggtaagccccagggtgtttgtgctcgtgttgcaattggtcaggttcttctatctgttcgctgcaaagatggaaacagtaaccatgcccaagaggctctgcgccgtgccaagttcaagtttcctggacgtcaaaagatcattgtcagcaggaagtggggattcaccaagttcagccgaactgattatctgagatacaagtcagagaaccgtattctcccagatggtgtgaatgcaaagcttcttggcaaccatggtcgacttgctgcacgtcaacctggaagggccttccttacatctgcttag

SEQ ID NO: 197 - DNA, 1314 bp, Solanum lycopersicum
  source 1..1314: mol_type = genomic DNA
atggaaagatatagaatattgaaggagcttggagatggaacatgtggtaatgtatataaggccattaataccgaaacatctgaaattgttgcagtaaagaaaatgaagaggaagttctatttttgggaagaatgcattaatctacgtgaaataaagtccctccgtaaattgaatcatcctaacatcatcaaactgaaagagattgtcagggaaaacaatgagcttttcttcatacttgagtacatggaatgtaacttgtaccaactaatgaaagatcggcaaagacctttcctagaggaagagattcgaggattgatgtctcaggtgttgcaaggacttacccatatgcataaaaatggttactttcatcgggacttaaaacctgagaatttactggtgacgaatgacgtaattaaaattgctgactttgggttggctcgtgaagtgtcttcatcacctcctttcactgactatgtttcaactcgttggtaccgagcaccagaagttttgttgcaatcttcatcatacacaccttctatcgatatgtgggcagttggtgcggtacttgctgagcttttcactctttgcccaatattccctggtgaaagtgaaattgatcagttgtataagatatgctgtgttcttggaccgccagattggactagcttcccagaactgagaagtgcttcacggttgtttgacattaattgttttgatattacaccagccaatctgtctgatttcattccaaatgctagcttggaagctatcgacttgatcaagcaactctgctcgtgggacccattgagaagaccaactgctgaccaatgcttacagcacccattttttcatgttgatatgtcaattcctcgtcctctagaggatccactgcagatgaatttaagtagtgttggacctgagcctaatcttgagttgaacctgtgggattttgggacagaaaaagatgactgctatcttggtttgactttggctgtgaacccaactccctcttgtctagatttatttccagcaaagatggcaagtaaaagtcaaggtgcaggaacggatatgttctgctctggtttccaagatcactcgcaacactcagttttctggtcattgtttcctactgatcgccatcagatatctactccggtggactcctcattgtcattatcattcagcacgattccacagtcaactattggagctccacaatcaactagttttggcatgacatctctgcagagtaacttcttggagcgcccctttctggctatgtcatcctcctttcagcagggacgttgcctttga

SEQ ID NO: 198 - DNA, 1431 bp, Solanum lycopersicum
  source 1..1431: mol_type = genomic DNA
atggcagcggaagcagcagtagcagtagcggcagtagcaccagtttctgctcaagttgttgcgaatgcttttgtgcagcaatactatcatatattgcatcattcgccggggcttgtattcaggttttaccaggatataagcaagcttggacgtcctgaagaggatggatccatgggtatcactacgacaatgcaagcaataaatgataagatactctcactcaactatggagatctcagagcagagatcaagtctgtggatgcccaagaatcctttaatggcggggtgcatgtccttgtaactggatatttgactgggaaagacaacttggtccggaatttctgtcaaactttcttcctagcaccacaagatagggggtactttgtcttgaatgatatgtttcgatatgtagagactgccagtcagcatgatacagtccaggttccagtaactgatgctgtggctcctgtcacaccagagcaaattgttgcagatccacctacagcaccacagaatcacattcctgagcaaagcactccaccagcagaggaagctgacgtgggagaagtttacaacccatctgagaatggtgatgtgcttattgttgaggaggaagtgcctgtgtctgaggtcgtccatgaagtccaagatggctctgaagtggtagcggaatctgacaacaaaactggggaagttcctaagaagtcatatgcttcaattgtcatgcatctcaaggaaagtgctgcaaccttctcccctcctccagcacctgctgctaggaagcctatgacaaagagtgttgtgcaagtgaatcagcctgctgccacaacaactgatggcccatattccagctcagagaccattgataacataaatgacacagaagctgatggctactcaatctatataaaaggcctgcctatgagtgcaactgtggcattacttgcggatgagttcaagaagtttgggccaataaaaaatggaggaattcaagtgagaagtaacaggcaacagggattttgttttggattcgtggaatttgaagtggaaagtgctgtgcaaaaagcaatcgaggcttctccagttctcattggtgatcggcaagcagtggtcgaggaaaagaggtctaccaattctcgacttaatgccagaggtggtggtaggtttcaatctggaaggggcggtggattcaggaacgatgttggaggaagagggcgaggaaactatttaggtggcaggggttatggtcgagatgatttccatggaagaaatgagttcaataacaggggtgggaatcgtgggggttcatcaaaccgtggaggtgaaggatatcggcgagctgacaatacaggtgggcgaatgagtcgtggtggtggaatgtctaatggaacagccaaaggcccggctcctcgctattctgctacagcttga

SEQ ID NO: 199 - DNA, 2628 bp, Solanum lycopersicum
  source 1..2628: mol_type = genomic DNA
atgcaattgagcttcagcagaccaaaatcaccaatgatgtttgatttgatttctatttctagccatcaccctaattccatcatcaatactataaataggccaatccaaaaccccaaaatcccatcattcttctcttctacaacaacccatttttcatactcattcaacaaatatagtaaccctttacaattctctgtttcatcacctaccacttcagttcttgctcacaaaggaagtgaagtagaatttgagaaagggctagaagaggaggaagcagcagcaaaaacagtagttgacgctgccatgacgattactccagcgataaggatttctgacagaaaactgatggtgaaagacagaacaatattaacaaatgtacccgacaacgtactcaccactcccggtgctgcatcaggtccattagagggtgtatttcttggtgcagaatttgatcaagacaacaaccgccatgttgtgcctctcgggaaattacaggatgttcggttcttgtcttgtttcaggttcaagttatggtggatggctcagaaaatgggtgataagggaagtgaaatcccaatggaaacacaatttcttcttgttgaaacaacagatggatctcatctcggatccaacgacaacaaaaatgatgataatattgtgtatgctgttttccttccattaattgaaggttcatttcgagctgttcttcagggtaatgctgaagacgagctcgaattatgccttgaaagtggcgataaggacactgttggatcagcatttaatcaagcagtttatattcatgctggcagtgatccgtttatcgtaatcacggaggcgataagggcggtgaagttacatctcaagactttccgccaacggcacgagaagaagctccccaaaattgttgattactttgggtggtgtacttgggatgctttttatcaagaagttactcaagaaggcgttgaagccggactcaaaagtcttacagctggtggtattccgccaaaattcatcatcatcgacgatggctggcagtcagtcggtggtgatccagaagttgataaaccattgatgagacttacaggattaaaagaaaacgaaaaatttcagaagaaagaagatccaacattggggattaaaaacattgtgaatatagctaaagagaaatacggcctgaactatgtgtatgtatggcacgcaataacgggttattggggcggagtccgacccggagttaaggggatggaggaatacggatcggttgtgaagtacccagatattacaaaaggagtgatggagaatgaaccgggatggaaaacggatgcaattgcggttcaaggtttgggtttggttaacccgaaaagtgcttataagttttataacgaaatgcatagttatttggcatcagctggagtggatgggttgaaggtggatgtgcagtgtatactggagactttagggggtggtttagggggtcgggttgagctcactaaacagtatcatcaagctcttgatgcttctgttgctcggaattttcctgataatggttgcattgcttgcatgagccacaatactgatgcactttactgttcaaagcagacggcagttgtgagagcatcagatgacttctatccaagagatccagcttcgcacaccattcacattgcttgtgtggcatacaacagtgtgttccttggagaaattatgctgcctgattgggacatgttccactcccttcatccagcagccgagtatcatggctcagcgagggcgttgagtggtggacctgtctatgtgagtgatgcacctggcaagcacaactttgatgttctgaggaagcttgttcttcccgatggttcaattcttcgtgctcgtttgcctggtcgacctacaaaggactcccttttcactgatccttctcgtgatggtgttagccttttgaagatatggaacatgaacaaatacactggtgttcttggaatatacaactgccaaggtgctgcgtggagcacggttgagaggaagaccacattccacaaaaccaactcagaggcgataacaggctatatcaggggctgtgatgtccatttcatatctgaagctgccttggaccccaactggagtggagacacagttctttactctcatggaagtgctgagcttgttgttctcccgtataatgcagcaatgcctgtttcttttaagatccttgagcacgagacatacactgtcacgcctattaaggtcttggctcctggcttcagctttgcacctttggggcttattgatatgtacaatgctggtggggcaattgaaggactaaaatacgaggtgaaggcaggcgcagaattgtctgaactcgaggctggatatcaaggtgaaggaaatcttgtggctgaagataaaattgagaacttgagcacagaagcagttgcagtagtgtcgatggaagtaaggggatgtggccgatttggtgtttactcatctgtcaagccgaggaagtgcagtgtgggtggagatatggttgattttgcctataactcagaatctggtttgttaactttgaatcttgatgctatgcctccagcagatcagaaagtgcacatcattgaagttgaagtatag

SEQ ID NO: 200 - DNA, 1575 bp, Solanum lycopersicum
  source 1..1575: mol_type = genomic DNA
atggcttcaacttgtctggatgtgagctttcttgggaatttttgcaacaactggagtttcagaccttgtacaagactaacaccttgcatttcccaagtaaggaatagtttattgcttgggaaaaacaagaactatactagctctgtaggatataagggaattccgttaacagaaatttcttgtgttccaagtgctgttgcgagttctagtgaacttcctccacttgttagtgctttaaaagcttcagctgaagaaaatgctgcaagttttcattttcctggccataatagaggacaagcagcgccatcatcactaactcagctcattggtgcaaaaccgtttcttcatgatatgcctgagcttccggagcttgacaatctgttttcaccagaaggtcccattctcgaagcacaaaaagaagcagcaagactttttggagcattggaaacatggttccttgttggcggtagtacatgtggagttcacgcggccattatggcgacttgttcacctggagatactcttattctcccacggaattctcatatctcagccatatctgccatggtattgtctggtgcacttcctaagtatatcgtccctgaatatgatttcaaatgggatgttgctggtggcatcactccatcacaggtgaatgatgcaatcaaagagctagaaatggaaggccgaaaagcagcagcccttttggtcgtctcccctacctatcatgagttgcctagctcatccctcagccagggagctgatcttgctgtacagtctactcacaaagttttgtgttcgcttacgcagtcatctatgttgcacatgcaaggaaatcttgttaatagagaaagaatcagcaaaagccttcaaatgcttcaaagtagtagccctagttatctgcttttagcatctctggatgctgccagggcccaattaagtgaaaatagagaggctgtttttgacaaagcaatggacttggctctggaagcaagaagcttgattagtaaaattcctggaatctcaatcattgagttcccaagtttctcaagttttttccagatcgacccattgcgactcaccattggtgtatggcagttaggtctttcaggttttgaagcagacgatatcttgtgcaatgattttggtgttgtttgtgaacttgttgggaccaaatcttttactttggcatttaacttagggactcaaagagatcatgtccttcggcttgtagctggtctaaaccatctatcacaaacttctcactttcctcaacctgtgaaagaagaaggcaaaaatgtaaatcattttgtatgccttgatgatattagaataagaatgagtcctagagaggccttctttgctacaaaacgtaaagtcagcataagagatagcctcggtgaaatatgtggagagcttgtatgtccttatccacctggtattccagttctaattcctggtgagatcatcacagcggaagcattgaattatctcctggagatcagaagcaagggtgctgttattaccggagcggctgatttctccctatcatcttttgttgtatgtgttacctaa

SEQ ID NO: 201 - DNA, 1770 bp, Solanum lycopersicum
  source 1..1770: mol_type = genomic DNA
atgagaagatctccaaatatgttccccaaatcgttcttcactctcctatttatcttcattttatctaccgccgtgtacggcggccgggtaatcgtcgatcagtcatcgtcggatctcatctccgatggagttcacggcggcggaggcagaaatagtactagtattggccgtgaaagaggaattatctcgctatttgctgcaccagtagaagaggcatgtgaacagagctatggatttttgccgtgcactaaaactgctctaggaaatgtgttccttattgttgtgtatggttaccttatgtttacggcggctacgtacctctcctccggtagcgagttattgcttgaaattttggggccgggtctaatcggtggacttttcctgccgattctcggtgctcttcctgatgctatgcttattctcgtatccgggattactggaagtgctgcagaagctcagagccaggtctctgtaggaataggcttgttagctgggtcgacagtgatgcttcttactgcgatatggggaacctgttgcactgttggcaagtgtgatatagtaaattctattgctgtggacttgaaagacactaaagggctcagttttactggttctggtgttagtactgatatctggacaagctatgctgcaatgatcatggccgtatctgttcttccatttattgttgtgcagttaccacagatcttccattcaaattcaggaagacatttatctgtcttgatagctcttatcatatcattgtcgctgctcctctcttattgtctgtatcaggtctttcagccttggatacaaacacgaaaacttgattatgtaaaacataagcatgttatatcaggaatcttgaagcatttaaaaaagcacgcattagggaggcttttcacagatcatggaacaccaaatatagaagttttagaaaagctgttcaatgcaattgatgaaaatggtgataaacatctttcccattctgaattaagagctctggtcgtaggaatccgtcttgaggagattaacttagatgaaaatgatgcaatagcaaaggttatgaaggacttcgatacgtctcttgatgaacaagttgacctgcatgaattcattactggagttgaaaaatggctttatgaagccaagagtactaatggttcttctgctcaggctggtcctaataccatcaactacattgatgattttcatgaggaaacaaggagagagcacagtctcttgggggaggatcaaagtgatgaaattgttgagggcgtcgagaatcctcgaaaagttgctattaaggccgcgtcattgttgttgttgggtaccattattgctgctgtctttgctgatcctttggtggatgctgttaacaatttctccagtgccacaagtattccatctttctttatctcattcattgcactgcctctggcaacaaactccagtgaggcagtgtcggctataatttttgctacccggaaaaagcagagatccgcgtcattaacattttctgagttatatggagctgtgactatgaacaatctcctttgtctctcagtcttcttggctattgtttatgcccgaggtctaacatggaacttctcatctgaagtgctggttatcctaattgtttgcattgttattggggtcttagggagcatccgcaccacctttcctctgtggactagtttgatagcttttgctttttatccgttctcccttgtactcgtgtatgttcttgactacgtctttggttggtcatag

SEQ ID NO: 202 - DNA, 1374 bp, Solanum lycopersicum
  source 1..1374: mol_type = genomic DNA
atgcagatctttgtgaaaactctcaccggaaagactatcaccctagaggtggaaagttctgatacaatcgacaacgttaaggctaagatccaggataaggaaggaattcccccggatcagcaaaggcttatcttcgctggaaagcagttggaggacggacgtactctagctgattacaacatccagaaggagtccaccctccatttggtgctccgtctacgtggtggtatgcagatcttcgtgaagactctcacgggtaagacgattacccttgaggtcgaaagctcagacaccattgacaacgtcaaggctaagatccaggataaggaaggcattcccccagaccagcagaggttgatctttgcaggaaagcagttggaagatggccgcaccctagctgactacaacatccagaaggagtccaccctccatttggtgctccgtctccgtggtggtatgcagatcttcgttaagactcttaccggaaagaccatcactttggaggtggaaagctccgacaccattgacaacgtgaaggctaagatccaggataaggaagggatccccccagaccagcagaggttgatcttcgctggaaagcagctcgaggatggtcgcaccctggctgactacaacatccagaaggagtctaccctccatcttgtcctccgtctccgtggtggtatgcagatttttgttaagaccctcaccggaaagaccatcactttggaggtggaaagctccgacaccattgataatgttaaggctaagatccaggacaaggagggaattccttcagaccagcagaggttgattttcgctggtaagcagctcgaggacggccgcacccttgccgactacaacatccagaaggagtcgacccttcaccttgtcctccgtctacgtggtggtatgcaaatctttgtgaagacccttaccgggaaaaccatcaccctggaggttgagagctccgacaccattgacaatgtcaaggccaagatccaagacaaggagggtattcccccagaccagcagaggttgatttttgctggcaagcagctcgaggatggccgcactttggcggactataacatccaaaaggagtcgaccctgcacttggtgcttaggctgaggggaggaatgcagatctttgtgaagaccttgaccgggaagaccatcactttggaggtggagagttctgacaccatcgacaatgtgaaagctaagattcaggacaaggaggggatcccaccagaccagcagaggttgattttcgctggtaagcagcttgaggatggccgcacccttgctgactacaatatccagaaggagtccaccctgcaccttgtcctccgtctccgtggtggtttttaa

SEQ ID NO: 203 - DNA, 1152 bp, Solanum lycopersicum
  source 1..1152: mol_type = genomic DNA
atgggctggctgcggtatgtggaatctgggaggaaaattgactgcattagcaatcctaacaccgcataccccattccagtccttgttgaagaagttgatgatattattagcgttgctcaatacgttatagttgtggagaaggaatcagtattccagcgactaggaaggggttatcctgatgttcccacaaggaggtttctgcgtctcctcattgacaagctgcatttgcctgtgtacggcctggtagattgtgatccatatggctttgacatcttaactacatacaaattcggttctttgcaaatggcttatgacgcaacatttctacgagtctctgagatacaatggcttggagtatttgtccaggattgtgacaattatagcattccacagcaatgtctcctgcctttaactgctgaagataagaggaaaatcgaagcaatgttacctagatgttatttgcaaagagaagtaccacaatggaggtttgaactggagctgctgttgcacaaaggagttaagtttgagattgaagcattgtctgtgcattcactcactttcttgtcccatgagtatctaccatcaaagatccacaatggagggtatatagtggacgttcattgtgatgcatgcaagatgaagatggtagatgttttgagttctatatgcggtgtttattctgtcacaatagactctgaagatgggacagccaagatttctggtgaagttgatccgaaccttctcttgagggcgttgtcaagatcaggccttggcaaccatgcagaggtgaagtgggtgagacttaagcacccaatgttgagtaatagtcatatgactgatggatacggttcctacaatcatggatatggctcttataatcatggatatggctctcataatcacggatatggttcgtataatcctggctacggttataacagttatggccacggtcatggccatggccatggctatagttctattggtggtccttttatgcaaagaagatcgttgcctgaatataattatggctatgagggacaccacaattaccaatatccttttagggggactgcacaagactcctatgccaccaactattactccggtgctctgccatcacctaggccgtacaacagctattatatgtag

SEQ ID NO: 204 - DNA, 2100 bp, Solanum lycopersicum
  source 1..2100: mol_type = genomic DNA
atgtcggacgtagagacgtttgctttccaggctgagatcaatcagcttctcagtcttatcatcaacactttctacagcaacaaggagatctttctccgtgaactcatcagcaattcttccgatgctctagacaagatccgctttgagagtttaaccgacaagagcaagctagatggtcaaccagagctcttcatccatattattccagacaaggccaataatactctcactatcattgatagtggtattggtatgacaaaggctgatctggtgaataacctgggtacaattgcaaggtcaggaaccaaggagttcatggaagctcttgcagctggtgctgatgttagcatgattggtcaattcggtgtaggtttctactctgcttacttggtagctgagaaggttgttgtgaccacaaagcacaatgatgatgagcaatatgtctgggagtctcaagccggtggctctttcactgttaccagggatacatctggtgagaaccttggtaggggtaccaagatggtcctttatctcaaggaggatcagcttgaataccttgaagaacgtaggctcaaggacctgattaagaagcactctgagttcattagctatcctatttctctgtgggttgagaagaccatagagaaggaaatttctgatgatgaggaggaagaggagaagaaagatgaggagggaaaggtagaggaggtcgatgaggaaaaggagaaggaagagaagaaaaagaagaaggtcaaagaagtttccaatgagtggtcactggtgaacaagcagaagcctatttggatgagaaagccagaagagatcacaaaggaagagtatgctgctttctacaagagcttgactaatgattgggaagagcatcttgctgtgaagcacttctctgttgagggtcagttggagttcaaggctgtcctttttgttccaaaaagggctccttttgacctctttgacacaaagaagaagcccaacaacatcaagttgtatgttcgccgtgtgtttatcatggataactgtgatgagttgattcctgaatatttgagctttgtgaagggtattgtggattctgaggaccttcctctcaacatctccagagagacattgcagcagaacaagatcctaaaggttattcgcaagaatttggtgaagaagtgtgttgagcttttctttgaaattgctgagaacaaggaggactacaataagttctatgaggcgttctctaaaaacctcaagcttggaatccatgaggattctcagaacagggcaaagtttgctgaactgctgaggtaccactccactaagagtggtgatgagatgaccagcttgaaggactatgtgaccagaatgaaggagggccagaatgatatttactacattactggtgagagcaagaaggctgttgagaactctcccttcctggagaaactgaagaagaagggatatgaggtgctttacatggttgatgccattgatgagtattcaattggtcagctgaaggaatttgagggcaaaaagcttgtttctgctaccaaggaaggcctcaagcttgatgagagtgaagatgagaagaaaaagcaggaagaattgaaggagaagtttgagggactgtgtaaggtgatgaaggatgtgctaggagacaaagttgaaaaggtcattgtttctgaccgtgttgttgactctccctgctgtttggtcactggtgagtatggctggactgctaacatggagagaattatgaaggcacaggcacttagggactccagcatggctggatacatgtctagcaagaagaccatggagatcaacccagagaactccatcatggatgagctaaggaagagggctgatgcagacaagaatgacaagtctgtgaaggacttggttctcttgctttttgagactgcccttctcacctcaggtttcagcctcgaggagccaaacacctttggcaacagaattcacaggatgttgaaactcggtttgagcattgatgaggaaagcggagatgctgatgctgacatgccagcattggaggatcctgaagctgatgctgagggcagcaagatggaggaggttgattaa

SEQ ID NO: 205 - DNA, 1614 bp, Solanum lycopersicum
  source 1..1614: mol_type = genomic DNA
ccatttgaactcggagataatagttacaagcataataaatacataatccaaacaattaatttctccatcatttcttctttgttcaccatggctactccaatcacaaaacccccatcacctgcttgtaccaaaatcatagaagaaatcacaaggatttacaaatcactaccaccaagaccatccattttagagattgaagcttcaatttctgttgtaaaatcagtagaaacagaagaaaaaatagaacttgatgaaatttcaaagagaagagttgagatagaagaaagtgttccagctgagctttactctgtgttagaaaaagtgagaaaagctatggttctgttccaaagcaaagaacagaagaaagaggctgttgagttgattgagcttgataagacttatcaagattttgatgagttgattcaggaagctacagagttggtttgtgaagatacccaaatgggaaaaatcaattcttttgaggacccaatggttgaaattggaaaaaaggatgaggttttgaagaagggtttggttacaagtggtgaattgattaatgtttcttcttcagggatcaagcacaaagagaagtacagcttgatgaaagtagctgccctaattgaaaatgccgccaaaactcgagcaagagttgttgatcttcagaacaagttaatggacaagattgaatggcttccattgtcacttgggaaattggtgaatgtcactgagctaaacgttgccgataaccagatcatggctcttccaactaccattggtagccttaatggcttaacgaagctcgatcttcactccaaccaaattataaaccttcctgattcatttggtgagctaatcaatttgactgatcttgacctccacgcgaacaggttgaaatcgctgccagcttctttcaggaacttagtaaatctgatcgatcttgatttgggttcgaacaggttcactcatttgcctgatttcgttgggaacttaacctccttgaagagactaaacgtggaaacaaatcagctcgaggaacttccttacactataggattttgttcttcccttgtagaactgagattggattttaatcagcttaaagctctccccgaggcaatgggaatgctcgaacacttggagattcttacgctacacattaacagaatcaaaggattaccaacaacaatggggaatctttctcgcctaagggaacttgatgttagcttcaacgaagttgagaacatacccgaaacattctgttttgctgtcagcttggaaaagcttaatcttgcaaacaactttgcggacttgaaaactttaccaagatcaatcggaaaccttgagaatcttgaagagcttgatattagcaacagtcaaataagaacgttgcccgactccttcaggctcttgtcaaagttgaaaacttttcgagctgatgagactccgctagaagtaccaccaaggcaaattataaagctcggggctcaggttgttgtagagtacatggctgaattcgtaactaagaacgagcttcaattgcagcggcctaagaggagacctttcttttgtccaagttgcctgtttcctaaaaccgaggggaagaggcggatctag

SEQ ID NO: 206 - DNA, 876 bp, Solanum lycopersicum
  source 1..876: mol_type = genomic DNA
atggagaaaatcaggaaagcatctcatgctggttcttggtacactgataatcccctagaactagaagaacagcttgatggctggctgagagctgctggcttagtcaagtcttctgatgtacgaggtgtaattgcacctcatgcaggctattcatattctggtcgtgcagcagcttatgcatttgggaacatagacccgaccaatatttctcggatatttctactcggtccatctcaccactattatactccaaaatgtgcactttcaagagccacagtttacaagactcctataggagatttacccattgatgaagaagtaaatgacgagctaaaagctacggggcactttgaatacatggatcttagagttgatgaagctgaacatagcatggaaatgcaccttccatatcttgctaaagttttccaagggtatccagtgaaaattgtgcccattctggttggggctcttagtgctgaaagtgaagccctttatggacggtttctcgcaaaatatgtggatgactcgaagaatttcttctcagtgtcttcggatttttgtcattggggatcaaggttcaactacatacattatgacaaaagccatggagctatttacaaatctattgaagtcttggataaaatgggtatggatattatagaaacaggagatccagatgcattcaaactgtacctgtcagagacggacaacaccatttgtggacgccatccaatcagtgtttttcttcatatgttgaaaaactcgtcgactaagataaagattagatttcttcgatatgaacaatcgagccagtgcaaatctatgagggacagcagcgtaagctatgcatcggcagtgggaaaagttgatggttga

SEQ ID NO: 207 - DNA, 3111 bp, Solanum lycopersicum
  source 1..3111: mol_type = genomic DNA
atggagcgtgctagaaaattggcaaacagagcaattctgaaacgtttggtttcacaatcaaaacagagtcgttctaatgaaatcccatcaccatcatcattatacagaccttcaaggtatgtatcttcactgtcaccatacacatttcaggctagaaatagtgtgaaatcttttaatactcaacaagttagatcaatttctgttgaggctttgaaaccaagtgacacattcccacgccgacataactcagccacccctgaagaacaaactaaaatggctgagttttgtgggtttcaaagcctagatgcacttattgatgctactgtacctcaatcgattcgtagtgaatcgatgaagttacctaagtttgatggtggattgactgagtcacagatgattgagcatatgcagaaattggcatctaagaataaggtttttaagtcatatattgggatggggtattataatacttatgtaccacctgttatattgaggaatcttttggagaatcctgcttggtatactcagtatactccttatcaggctgaaatttcccagggacgtcttgagtccctgctgaattatcagaccatgattacggatcttactggtttgccaatgtctaatgcatctttactagatgaagggaccgccgcagctgaggctatggctatgtgtaacaatattcttaagggaaaaaagaaaactttccttattgctaacaattgtcaccctcagactatcgagatttgtaagactagagctgatggatttgatcttaaggtagttactgtagatcttaaggatattgattataagtctggtgatgtttgtggggtactagttcagtatccggggaccgaaggtgaaatcttggattatggggagtttattaagaatgcacatgctcatggggtgaaggttgttatggcatctgatcttttggccttgacaatgttgaaaccacctggtgagcttggagcggatattgttgttggttctgctcagaggtttggagtgcctatgggttatggaggccctcatgcagctttcttggcaacttctcaagaatacaagagaatgatgcctggaagaattattggtctcagtgttgattcaacagggaaacctgctctgcgtatggcaatgcagactagggaacaacacatccgcagggacaaggctacgagtaacatttgcacagcacaggctttgcttgccaacatggctgccatgtatgctgtctatcatggacccgaggggctaaaaaccattggccaacgtgttcatggtcttgctggaacattttctgctggtctcaaaaagctaggaacagtagaagttcaggatcttccattctttgacactgtgaaggtgaagtgttctgatgcaaaggcaattgctgatgttgctaccaagaatgacattaacgtgcggattgtggacaacaatactataactgtatcttttgatgaaactactaccctagaagatgtggacgatctattcaaagtttttgccttgggaaagccagtcccgttcactgctcaatcaattgcacaagaggttgagaacctgattccttctggacttacaagggagactccatttttgactcaccaaatattcaactcgtaccatactgagcacgagttgctgagatacctccataagctgcaatcaaaggatctctccttgtgccatagcatgattcctttgggctcctgcacaatgaaattgaatgccacaacagagatgatgccggtgacatggcctagctttgcaaatattcacccttttgcacccactgaacaggcagctggctatcaggaaatgttcgacgatttgggcgccctactgtgtacaataacaggttttgattccttctcgttgcagcctaacgctggtgctgctggagaatatgctggattgatggttattcgtgcatatcatatgtcaaggggtgaccatcaccgcaatgtatgcatcattcctgtatcagctcatggaacaaatcctgcaagtgctgcaatgtgtgggatgaaaattgttgctgtcgggacagatgcaaaaggaaacattaatattgaagagttgaggaaggctgctgaggcacataaggataacctttctgctctcatggttacatacccatcaacacatggagtgtatgaggaaggaattgatgagatatgtaagattatccatgacaatggtggtcaggtgtacatggatggagctaacatgaatgcacaggttggtttgacaagccctggctttattggtgctgatgtttgtcatctaaatcttcacaaaacattctgcattcctcatggtggaggaggtcctggaatgggtccaattggagtgaagaaacacttggcaccatatttaccatcacaccctgtggtatcaactggagggatcccatcccccgatcagagtaagccacttggtgctatttctgctgcaccctggggttcagcacttattttgccgatttcatatacctacattgccatgatggggtctaagggacttacagatgcatcaaagatagctatcctgagcgcaaactacatggcgaaacgtttggagaagcactacccagtcctcttccgaggtgtcaatggaacatgtgcccatgagtttatcattgacctgaggggctttaagaacactgctgggatagaacctgaagatgttgctaaacgtcttattgactacggatttcatggacctacaatgtcttggccagttcctggtacacttatgattgaacctactgaaagtgaaagcaaggcggaactagacaggttttgtgatgcactcatctccatcagagaagaaatcgctcagattgagaaagggaatgtcgatattaacaacaatgttctcaagggggctcctcatccaccatcaatgctcatggcagatgcatggacaaagccatattctcgggaatatgctgcataccctgctccctggctaaggagtgccaaattctggccaactacaggacgagtggacaatgtgtatggagatcgcaacctgatctgcacccttcttccagtgtcagaaatggcggaagaaaaagctgcaactgcttaa

SEQ ID NO: 208 - DNA, 456 bp, Solanum lycopersicum
  source 1..456: mol_type = genomic DNA
atgcatgttgaaccccacaggcgagccctgagcgttgagcgttgggtgtgcttagggcgtgcaatcggacgcttagggccccactttttgtttcaacagaacaccaaatcgagctctttcttcttcactgcactgttgttttccgctagggtttcaggtattagtaggtgtgtattgaaaatgtcaagagtctatgttggaaatctggaccctagggtcagtgaaagagagcttgaagatgaattccgcatctttggagttataagaagtgtttgggttgcaagacgccccctggctacgcttttattgacttcgatgatcggcgggatgcacaagatgcaatcaaagagctggatggtaagaatggatggagagtggagctttcacataattctagaggtggaggtggtgggggccgtggaggaggtcgaggtcgatctggaggctctgatttga

SEQ ID NO: 209 - DNA, 1293 bp, Solanum lycopersicum
  source 1..1293: mol_type = genomic DNA
atggactatggttgcttaaaaaaaatagccacaaaatcccccaccactacgatctttcttccctatttgctccgatctgtgttaaccacacgctttcaattgcttgtggccggctcattctgcaactcctccggcgaccatcggaattcggaagtttttacctctttttccggatcaaaagaaatggagttcttcttcagaacacagactgaggagacatctgattgctccttcaatggccaggatattcaaagatgcccgttcttgagtaacatcaacaagccaacaaacttttctttcttttctgctctgaactttcccagccctgtgaagggaggtaaaggtccaatttttgaagatggccccaacttcgatatggcatttaaagtcttccatggaaaagatggggttgtcccactttctggaaggtctcaatttttcaatgacaacattgaagtagagtctgcccctcaatttaatcctttagctgcaaaagctgccaccataagtttgtcttcattcggagcaggaggtccttttagctttgattctttctctcggaaatggaattcacagaagaagaagccggagtcatccaaaaagaaaaagccttcttctcaggacaaatcttctaagcacgaggcaatgggaaatgagtggttggagtcaggcaactgtcctatcgccaaatcttatagagctgttagcggtgttctcccacttgtggcctcagcttttcagctgcctcccggtatgaagctcaaatgcccgcctgcagttgttgcagccagggctgccctcgccaggactgcttttgtgaagactgtgcggccgcaaccactatcttcaaagatgcttgttattggagctttgggcatggcagctaatattccactaggcatatggagagagcacactgagaagttctcactttcatggtttactgcagtccatgctgctgttcccttcatagctatgctgaggaagtcagttgtgatgcccaaaacagctatggcattaaccattgctgcttctatcttgggacaggtcattggctcaagagcagagagacttcgaatgaaagcaaaagccgagagtgttaaattggtagcgcagactggctcagatggggttattgcacagagtagcacagagggtgttattgcaggtctcaactcaatccaggtcagtggtatgcccggggttcattgtggcacacagggtatgctaaaagaccaaccctcgaaagaatctgccaatactatatctccatctgccagtctctgtttctaa

SEQ ID NO: 210 - DNA, 957 bp, Solanum lycopersicum
  source 1..957: mol_type = genomic DNA
caaagttttctcactcaaataataaatataataaatagttgtttcagtatgaaaacggtccttttccgaaccggttccggctcaattccggcggttgttcccggttctcagagagcagtctctgtttccactcgtgaatccatcggtaaagtgtttaatggagagaagaagaaaggatgttcttctcctatgatctctctgtattcggaagtcaatcgtcgcggtattcgccgggtttcatctgactccgacgtgatccggtcgggatttgaaggttcgagtgtaactagaaagttgagtaagattggatcgaggtcattatcgtcaataataccggaggaggattgcggttctgaacagaatgagttggaggttttgcgaagtattggatcactgagtttgacggaagatcgacggagttacgctggagactggcataggaacagtattccagtcgatgagcttggattttccggcggtggaatcggttccaccaaaaacaaaaagtttggcggcaatggtgacggtagagatgagatcgatgcagatagtttaaccggcggaaagtcagatccaaggaaaattggagcctattataaggaaatgctgaattcagatcctatgaatcctcttcttcttagaaactacggcaagtatttgcatgaggtggagagagactttgtgaaggctgaagaatgctacggaagagctatacttgcaagtcctggtgacggagaagtgctttctctgtatggcaaattagtttgggagacgcagcgagacgaaaacagagccaaatcttactttgatcaagctgttcacgcttctcctaatgactgcactgtgttgggatcatatgcacacttcatgtgggaagccgaaggagaccaagacgaagacgaagacgaagaaagagaaacagtagcagctggagcatcaatggttgcagcttactag

SEQ ID NO: 211 - DNA, 1458 bp, Solanum lycopersicum
  source 1..1458: mol_type = genomic DNA
atgagtagtattacttattctaatttgatattgaagaggagagaatcaagaatcaatggtggatctcatttacaatataatggacttagagctattgaaactgtgaaattgacagcaaactctgcttcttgtaagccaaaagctgtaaaatgtggaagaatcagagccatggcctctccagctgtttcagctcccaagagagagacagacccgaagaaaagggttgtcataaccggaatgggacttgtttcagtctttggaagtgatatcgacaatttttacaacaaacttcttgaagggcagagtggaatcagtttaatagactcttttgatgcctcaagttacccagttaggttcgcgggacagattcgtgatttctcttccgaaggttacatagacggaaagaatgatcgtcgtttagataactgctggagatactgtctcgttgctggaaagagagcccttgacgatgctaaccttggccaacaagttcttgaaactatggacaaaacgaggatcggtgtcttggttgggtcggggatgggaggtttaaatgttttcagtgacggggtggaagcgttgttgcaaagaggatacaagaaagttagtccatttttcattccttactctatcaccaacatgggatcagcattgttggctatagaaaccggactaatgggaccgaattactctatttcaacggcttgtgcatctgccaatcacagcttttgcgcagctgcgaatcatataagaaggggtgatgcagatattatggtaacaggtggaacggaagctggtgttacagctactggtgttggtggcttcatagcttgtcgggccttgtctcaaagaaatgatgattatgaaaaggcttcaaggccatgggacacgaatcgtgatggatttgtcgttggtgaaggctctggtatcctggtcatggaaagtttggagcacgcgttgaaaagaggtgctagtatcatcgcagagtacttgggaggagcagctacttgtgatgctcatcacatgaccgatcctcggcctgatggacttggagtttggtcttgcattactaagggtttgttagatgccagaatttctccagaagaggtgaactatataaatgctcatgcaacatcaactcttgctggagatttggctgaggtcaatgccatcaagaaggtcttcaaagatacctcggaaatcaaaatgaacgggacaaagtcgatgatcggtcatggacttggggctgctggtggaattgaagccattgtaaccatcaaagcaatcacaacaggatggcttcatccaaccatcaatcaacatgaattagaaccccaggtcacgattgacaccgttccaaatgtgaagaaacaacatgaagtgaacgttggtatctccaactcgttcggattcggaggacataattcagtggttgtttttgcaccttacaggccttga

SEQ ID NO: 212 - protein, 1173 aa, Solanum lycopersicum
ATGGAAACTTTCTTGTTTACCTCAGAGTCAGTCAACGAAGGTCACCCCGACAAGCTCTGTGACCAGGTCTCAGATGCCATTCTTGATGCTTGCCTGGAGCAGGATCCCGAAAGCAAGGTTGCATGTGAAACCTGCACAAAGACAAACATGGTTATGGTCTTTGGAGAGATCACAACCAAGGCCACCGTAGACTACGAGAAGATCGTACGTGACACATGCAGAGGCATTGGGTTCGTCTCAGCAGATGTTGGTCTTGATGCTGACAACTGCAAGGTCCTTGTTAACATTGAACAACAGAGCCCTGACATCGCCCAAGGAGTTCACGGTCATCTTACCAAGAAACCAGAAGAAATTGGAGCTGGTGACCAAGGTCATATGTTTGGGTATGCCACAGATGAAACTCCTGAGCTCATGCCCCTCACCCATGTTTTGGCCACCAAGCTTGGTGCCAAGCTTACTGAAGTGAGGAAGAACAAAACCTGCCCATGGCTCAGGCCCGATGGCAAGACCCAAGTTACTGTTGAGTACAAGAACGACAATGGTGCCATGGTCCCTATTAGAGTTCACACTGTTCTCATCTCAACCCAGCATGACGAAACTGTCACCAACGACCAGATCGCCCAGGACTTGAAAGAGCATGTGATCAAGCCTGTGATCCCAGCTAAGTACCTTGACGAGAACACCATCTTCCACCTCAACCCATCAGGTCGCTTCGTCATTGGTGGTCCACATGGAGATGCTGGTCTTACTGGCAGGAAAATCATCATTGATACCTACGGAGGCTGGGGTGCTCACGGTGGAGGTGCCTTCTCAGGAAAGGACCCCACTAAGGTGGACAGGAGTGGTGCTTACATTGTTAGGCAGGCAGCAAAGAGTGTGGTCGCCTCAGGACTTGCTCGCCGCTGTATTGTGCAGGTTTCTTATGCTATCGGTGTGGCTGAACCACTTTCCGTGTTTGTTGACACATACAAGACAGGAACAATTCCCGACAAGGACATTTTGGTTCTGATCAAGGAGAACTTTGACTTCAGGCCTGGAATGATGTCAATCAACCTTGATTTGTTGAGAGGAGGCAACTACAGGTACCAGAAGACTGCAGCTTACGGTCACTTTGGCCGTGATGACCCCGATTTCACCTGGGAAACTGTCAAGGTCCTCAAGCCAAAAGCTTGA

SEQ ID NO: 213 - protein, 1335 aa, Solanum lycopersicum
ATGGCAACTATCAAATCCATTAAAGCTCGTCAGATCTTTGACAGCCGTGGTAACCCCACAGTCGAGGTTGATGTACATATCTCAAATGGTGTGTTTGCCAGAGCTGCTGTTCCAAGTGGTGCATCCACCGGAATTTACGAAGCCCTTGAATTGAGGGATGGAGGATCTGACTACCTCGGAAAGGGTGTTTCGAAGGCTGTTAACAATGTCAACTCAATTATTGGCCCTGCTCTTGTTGGCAAGGACCCAACTGATCAGACTGGTCTTGATAACTTCATGGTTCATCAGTTAGATGGAACTCAAAATGAGTGGGGTTGGTGCAAAGAAAAGCTTGGTGCAAATGCCATCCTTGCTGTGTCACTTGCTGTGTGTAAAGCTGGCGCTGCTGTCAGGAATGTGCCATTGTACAAGCACATTGCTGATTTGGCTGGTAACAAGAAGTTGGTATTACCAGTTCCTGCCTTCAATGTGATTAATGGTGGATCTCATGCCGGAAACAAACTCGCAATGCAGGAATTTATGATCCTTCCTGTTGGAGCTGCTAATTTCAAGGAGGCCATGAAGATGGGCTGTGAAGTCTATCACCATTTGAAGGCTGTGATTAAGAAGAAATATGGTCAGGATGCCACAAATGTCGGTGATGAGGGTGGTTTTGCTCCTAATATCCAGGAGAACAAGGAAGGTCTTGAATTGCTCAAGACAGCCATTGAGAAAGCAGGTTACACTGGAAAGGTGGTCATTGGAATGGATGTTGCAGCATCTGAATTTTACGGAAAGGACAAATCTTATGACCTGAACTTCAAAGAAGAGAGCAATGACGGCTCACAAAAGATATCAGGTGACCAACTCAAGGACTTATACAAGTCATTTGTGTCCGAGTACCCTATTGTTTCAATTGAAGATCCATTTGACCAAGATGACTGGGAGACCTATGCTAAGCTCACTGCTGAGATTGGGGAGCAAGTACAGATTGTCGGTGATGACCTTCTTGTCACCAACCCTAAGAGGGTCGCCAAGGCAATTGCAGAGAAGACTTGCAATGCTCTTCTTCTCAAGGTTAACCAAATCGGTAGTGTGACCGAGAGTATTGAAGCTGTGAAGATGTCCAAGAAGGCAGGTTGGGGTGTAATGACCAGTCACCGCAGTGGAGAAACAGAAGATACCTTCATTGCTGATCTTGCTGTCGGTTTGTCAACGGGACAAATCAAGACTGGAGCTCCTTGCAGGTCAGAGCGTCTCGCCAAGTACAACCAGCTGTTGAGGATCGAAGAGGAACTCGGATCAGAGGCTGTTTATGCAGGAGCAAGCTTCCGCAAGCCCGTTGAGCCCTACTAA

SEQ ID NO: 214 - protein, 330 aa, Solanum lycopersicum
ATGGATGCTCAATCCAAATCAGACTCTTTTTCATCTTCAAACCTAAGAATCATTGTCCAAAACAACCCTTCAGAATCCCAACTCTCTGAATTGGGCATAAAATCTTGGCCAAAATGGGCTTGCTCACCAGGGAAATACCAATTAAAATTTGATGCAGAAGAAACATGTTATTTGCTAAGAGGAAAAGTGAAAGTGTATCCAAAGAACACAATAGAGATGTCACCAGTGGAGTTTGGTGCTGGGGATCTTGTCATTATTCCCAAAGGACTTTGTTGCACTTGGGATGTAACCCTTCCTGTTGATAAACACTATAAGTTTCATTCTTCCTAA

SEQ ID NO: 215 - protein, 1917 aa, Solanum lycopersicum
ATGTTCCCTTTGCTTCATCTCTTCATTTTTCTTTGCCTTTCATTCTCTTATTGCTGTTATGGACAAATCATCAATTCTTCCTCTTCTTCATGTCCTCTCGATTTCACTTCACTTCGCAAAATCATAGATCAAAATTCAAAAAGACCCACTTTGAATTCCACCGCACAGTGTCAGTACATTCGTCAGGGTTTACGTCTAGTTCAATCGGAGTATCTCCGCCGTACCAACTCTTTCTTCCCGCCTATGAGTTCCGCGGATTCTTGCTGGAACTCTTATCAGTCTCTGGTCAACGATTATGTCCAAAACTTTGATATTCGGAGTTCATGTGGGTTTCAAACAGCTTGGATCTCTCAGGGTTGTATGAATATCACAACCCGTTTCGAGTTCGAGTCTAAGGTATCCCCTGCTGCGATTACTAATATTGTTTCTTCCTGTAATCAGTCTTTAGAGAACAATTCCCCTTGTGCTACTTGTACAACTAGTTTATCTGGGCTTGCTTCGTTTCTACCGGGTCCTTCTGTTGGAAATTTGTTCGATTGTGCGGCTTATCCTTCGGTTTATGCTGCTGCTTTTGCTAATCAGTTTGGACCTACTGATAAGGGTACTGCAAAATGTTTGTTTCTTCTTGATGTTAGTGTTGGTAGCTCAGGGAAGGGTAAGAAGAGTGTGGTTGTTATTGTGGTGGTGGTGGTTTGTGTGGTGGTTTTCGCAGTTGTTGTGTTTGGTTATTGGTTTATGTGGAGGAAGAGGAAAATTGGGTTGGCAAAAAAGTGGAATACTAAGAGATTGGAGAGTAACTTGAGTTCTCGTTTGGATTCGATTAGTGGAAGTACTACTTTGATTAGGTATAGTTTTGATGAGATTAAGGCGGCTACAAAGAATTTCTCTAGGTTGAATATAGTTGGGACAGGAGGGTATGGGAATGTGTATAAGGGTGTGTTGCCGGGTGGGATTGAAGTAGCATTGAAGAGGTTCAAGAATTGTTCGGTTGCTGGTGATGCGAATTTCACTCATGAAGTGGAGGTTATTGCTAGTGTTAGGCATGTGAATTTGGTGGCTTTGAGAGGGTACTGCACAGCGACAACCCCTTTTGAGGGTCACCAGAGGATCATTGTTTGTGATTTGATGAAGAATGGGAGTCTTCATGATCATTTGTTTGGAACGAGACATGAAAAATTGAGTTGGGGTATTAGGCAGAAGGTTGCAATTGGCACTGCTAGAGGATTGGCTTACCTGCATTATGGAGCACAACCGGGTATTATTCACAGGGATATTAAGGCTAGTAATATACTTCTAGATGAGAGTTTTGAACCTAAGGTGGCGGATTTTGGATTGGCTAAGTTCACTCCTGAAGGGATGACTCATTTGAGCACTCGTGTAGCAGGAACGATGGGGTATGTAGCACCTGAGTATGCTTTGTATGGCCAATTGACTGAAAGGAGCGATGTTTATAGCTTTGGAGTTGTGCTTCTTGAGCTTTTGAGTGGAAAGAAAGCAATAATGGAGTTTAAGGATGGGCAGCCGACGCTTGTGACTGATTGGGCCTGGTCATTGGTTAGGGAAGGCAGAGCATTAGATGTTCTTGAAGACAGCATTCCGCATTTGGGGCCTCCAGAAGTTATGGAGAAGTATGTATTGGTAGCTGTTCTTTGTTCTCATCCAGAATTGTACGCGAGGCCAACAATGGATCAGGTTGTGAACATGTTGGACGCTGAGATACCTGTTCCAACCATACCGGAGAGACCAATTTCTCTCATTGCTGATCTTGATGACATTGAAAGGTCTGTTAGCAGTGGTGGTAACTCAGGCAATCTGTCCACTGCTGCAGGCTATCAGCCTTACATATTTGAGCGCGAGACACCTCTGGCAGATAGTGATTCTGTCAGGGCAAGAACATTGGATCAGAGATCGCGATAG

SEQ ID NO: 216 - protein, 918 aa, Solanum lycopersicum
ATGAGGAACATGCTGTTTGCTTCACTATATCCCGCCCGACTTCATTTCCTTAAAACCCTAATCTTCAAAATTTCCTCTTCTTCTACGCTTTCTGCTCTCGATCTTCCCAGTCGGTCGAGTAGGACACTTTATGTTGGCAATCTTCCTGGTGATGTTCGTGAGCGTGAAGTGGAAGATCTCTTTTACAAGTATGGACCGATTGCACATATTGAGCTGAAAATTCCACCAAGACCCCCTGGTTATGCTTTTGTTGAGTTTGAAGAGGCTCGTGATGCTGAAGATGCTATTCGTGGTCGTGATGGCTATGATTTTGATGGGCACCGTCTGCGGGTTGAGCTTGCACATGGCGGGCGTGGTAACTCATCATCAAATGATCGTTATGGTGGTGGCGGTGGCGGCCGTGGTCAGCGTGGTGGAGGAGTGTCAAGACGTTCAGATTATCGAGTGTTAGTTACAGGATTACCCCATTCAGCATCGTGGCAGGATCTCAAGGATCACATGCGCCGTGCTGGGGATGTTTGTTTCTCCCAAGTTTTCCGTGACGGGAGTGGAACCACTGGTATAATTGATTATACAAACTATGATGACATGAAATATGCTATCAAAAAGCTTGATGACTCTGAGTTTCGAAATGCCTTTTCTCGTGCGACAATTCGAGTTAAAGAATATGACCGCAGTCGAAGTCGCAGCCGCAGTCGCAGTCGCTCTTACTCCAGAGGGAAGAGTGTTAGTCGAAGCCGAAGCCGTAGCCGAAGTAGAAGCCGAAGCAAGAGCAAAAGCAAATCTCCTAAAGTTAAGTCTTCAAAGCGCTCAAGATCTCGTTCAAGGTCTGTGTCTTCTCAGTCTCGTTCTGGGTCAAAAGGACGTCCTGTCTCAAGATTTTGGATATGTGCGGCGGTTTGTGGATTTGCATATTAG

SEQ ID NO: 217 - protein, 1458 aa, Solanum lycopersicum
ATGGCTCTACTCGTTGAGAAGACCACCTCTGGCCGCGAGTACAAGGTGAAGGACATGTCTCAGGCTGACTTCGGCAGGCTCGAAATCGAGCTTGCTGAAGTTGAAATGCCTGGTCTCATGGCTTCACGTGCTGAATTTGGCCCTTCACAGCCCTTTAAAGGTGCAAAGATCACTGGATCTCTGCATATGACTATCCAAACTGCTGTCCTTATTGAAACCCTAACTGCTTTGGGTGCTGAAGTTAGATGGTGTTCTTGCAACATCTTCTCAACTCAGGACCATGCTGCAGCAGCCATTGCACGTGACAGTGCTGCTGTCTTTGCCTGGAAAGGTGAGACTTTGCAGGAGTACTGGTGGTGTACTGAGAGGGCACTTGACTGGGGTCCAGGTGGTGGTCCTGATCTGATTGTTGATGATGGAGGTGATGCTACTCTGTTGATTCATGAGGGAGTTAAGGCTGAAGAGGAGTTTGCTAAGAATGGAACAGTCCCAGATCCCACTTCTACTGACAATGTTGAGTTTCAACTTGTGCTTACTATTATTAAGGAGAGCTTAAAGACTGATCCATTAAGGTACACTAAGATGAAGGAGAGACTTGTTGGTGTTTCTGAGGAAACTACCACTGGTGTTAAGAGGCTTTACCAAATGCAGGCTAATGGATCTTTGCTTTTTCCTGCTATCAATGTTAATGACTCTGTTACCAAGAGCAAGTTTGACAACTTGTATGGATGCCGCCACTCACTTCCCGATGGTCTCATGAGGGCTACTGATGTTATGATTGCTGGAAAGGTTGCTCTTGTTGCTGGTTATGGAGATGTCGGCAAGGGATGTGCTGCTGCCATGAAACAAGCTGGTGCCCGTGTGATTGTGACTGAGATTGACCCAATCTGTGCTCTCCAGGCTACCATGGAAGGCCTCCAGGTTCTTCCTCTTGAGGATGTTGTTTCTGAGGTTGATATCTTTGTGACCACCACCGGTAACAAGGACATCATCATGGTTGACCACATGAGGAAGATGAAGAACAATGCCATTGTCTGCAACATTGGTCACTTTGACAACGAAATCGACATGCATGGTCTTGAAACCTTCCCTGGTGTGAAGAGGATCACAATCAAGCCACAAACCGACAGATGGGTCTTCCCAGACACCAACAGTGGCATCATTGTGTTGGCCGAGGGTCGTCTCATGAACTTGGGATGTGCCACTGGACACCCCAGTTTTGTGATGTCTTGCTCTTTCACTAACCAAGTCATTGCCCAACTCGAGTTGTGGAATGAGAGGAGCAGTGGCAAATACGAGAAGAAGGTGTACGTCTTGCCAAAGCACCTTGACGAGAAGGTTGCTGCCCTTCATCTTGGAAAGCTCGGAGCCAAGCTTACCAAACTCACCAAGGATCAAGCTGACTACATTAGCGTACCTGTTGAGGGTCCTTACAAGCCTGCTCACTACAGGTACTGA

SEQ ID NO: 218 - protein, 1041 aa, Solanum lycopersicum
ATGACGAAAGAGGGAAAGAATGTGAAAAGAATCAAATTGGGTTCAGAGGGACTTGAAGTATCTGTTCAAGGACTTGGTTGTCTGGGTATGTCCGATTTCTACGGTCCGCCCAAACCCGAACCCGATATGATTGAACTAATACACCATGCCATTAACTCCGGCATAACATTTTTCGATACTTCAAATAGTTATGGGCCTTACACTAATGAAATCCTCCTTGGCAAGGCTTTGAAGGGAGGAATGAGGGAACGAGTCGAGTTAGCAACAAAATTTGGTATACATTTTGCAGATGGAAAGATAGAAGTACGTGGAGAGCCAGAATATGTAAGGGCAGCATGCGAGGCTAGCTTAAAGCGACTTGATGTTGATTGCATAGACTTGTACTACCAGCACCGGATTGATACACGCGTGCCCATTGAAGTAACGGTTGGAGAACTTAAGAAGCTGGTTGAAGAGGGTAAAATAAAATATATAGGTCTATCAGAGGCTTCAGCATCAACAATTAGAAGAGCACACGCTGTTCATCCAATAACAGCAGTACAATTAGAATGGTCACTATGGTCGAGAGATGTAGAGGAAGAAATAATCCCTACTTGCAGAGAACTCGGAATTGGGATTGTAGCATACAGTCCACTAGGCCGGGGCTTTTTGTCATCCGGTCCAAAGCTGGTTGAGGACTTGACAAATGAAGATTTCCGAAAGCATCTACCGAGGTTTCAGGGCGAGAATCTTGAGCATAACAAAAAATTGTACGAGAGGATTTGTGAATTGGCAACTAAGAAGGGATGCACCCCGTCTCAACTGGCGTTGGCGTGGGTACATCACCAAGGAAACGACGTGTGTCCCATCCCGGGCACCACTAAGATTGAAAATTTCAATCAGAACATTGGAGCTCTGTCCATAAAGCTAACATCAGAAGACATGGCAGAGCTTGAATCTATTGCTTCAGCTAATGCAGTCCAGGGTGATAGGTACGGTTCGGCTGCAGCAACCTACAAAGATTCAGAAACTCCTCCTATTTCAGCATGGAAGGCTACATAA

SEQ ID NO: 219 - protein, 1218 aa, Solanum lycopersicum
ATGAAGAACGGTATGGCAGAATGCGCTGTCTGTCATTCCAAGCTTGTTTCTCCATCTGTGAAAACTATTTCAAGGGCATATGATCGACACAGAAGCAAGATTTCATCAAAGCAACGTGCTCTCAATGTCCTATTGGTTGTAGGAGATTGTATGCTGGTTGGTTTACAGCCTGTTTTGGTATTTATGTCGAAAGTGGATGGAAAATTCAAGTTTAGTCCCGTAAGTGTCAACTTCTTGACAGAGGCGACAAAAGTCGTCTTTGCAATAATAATGCTTTTAATCCAGGCCAGGCATCAAAAGGTTGGAGAAAAGCCACTTCTTTCAATATCCACATTTGTCCAGGCTGCAAGAAACAATGTCCTTCTTGCTGTACCAGCACTACTCTATGCCATAAACAACTACCTAAAGTTCATCATGCAGTTATATTTCAATCCAGCAACAGTGAAGATGCTAAGCAATCTGAAGGTTTTGGTTATTGCAGTGCTGTTGAAATTTATCATGAAAAGGCGATTTTCCGTAATTCAGTGGGAGGCTCTTGCTTTGCTGCTAATTGGGATAAGCATTAATCAACTTCGATCCCTTCCTGAAGGCACCACTTCTTTGGCTCTTCCAGTTACCACAATTGCCTACATCTATACACTGATTTTTGTAACTGTGCCTTCTATGGCCTCAGTATTCAATGAGTATGCTTTGAAGAGTCAATATGACACTAGCATATATCTCCAGAACTTATTTCTCTATGGTTATGGTGCTATATTCAACTTTCTAGCCATTCTTGGGATTGCTGTATTTAAAGGTCCTGGTAGCTTGGATATTTTTCAGGGGCATTCGAAGGCAACCATGCTTCTAATTGTTAACAATGCAGCGCAAGGAATTCTGTCGTCCTTTTTTTTCAAATATGCTGATACAATTCTGAAGAAGTATTCTTCGACCGTTGCAACAATATTTACAGGCATAGCATCTGCTCTGCTGTTTGGTCATACACTGACCGTAAACTTTCTTCTTGGGATTTCTGTTGTTTTCATTTCAATGCATCAGTTCTTTTCACCTCTTTCAAAAGTTAAAGATGATCAACAAAACGGGACAGTGGAGTTGATTGACGTCCGGGAGAACCATAGCCCTAAAGATTCATCCTTTGTAAATATGGCAGCAGGAGCAAATGAGGAGGCAAGTCATCGAGTAGGACCTGATGAAAGGCAGCCACTTCTTCCAAGATGA

SEQ ID NO: 220 - protein, 1260 aa, Solanum lycopersicum
ATGGCTTTTTCTTCTCTTCTCAAACCTGCCGCCTCCTTTGTTCGACCTTCACATCGATCTCAGGTATCATGTGCAGGACTTCATCATAGTAGTAATTCTGTTAAATTACAATCGTCTATCTTCGGAGATGCTGTGACAATTTTGCAATCTTCGTCTTTACAAAAATCTGGTGCCTGCAGTATTCAACCTATCAGAGCAACTGCTACTGAGCTGCCTCCAACAGTTCCAAGGTCACAAACTGGTGGGAAGACAAGGGTTGGCATCAATGGTTTTGGACGTATTGGGAGATTGGTATTACGAATTGCAACATTCAGGGATGATATTGAAGTGGTCGCAGTTAATGACCCATTTATCGATGCAAAGTATATGGCTTACATGCTCAAGTATGATTCCACTCATGGAGTCTACAGTGGATCCATCAGTGTCCTGGATGACTCTACTTTGGAAATCAATGGGAAGCAGATTAAAGTCAGTAGCAAAAGGGATCCCGCAGATATTCCATGGGGTGATTTAGGTGCAGATTATGTTGTTGAATCTTCTGGTGTCTTCACAACTATTGATAAGGCATCAGCACATAAGAAGGGTGGTGCAAAAAAGGTCGTAATCTCAGCACCATCAGCTGATGCACCTATGTTTGTGGTAGGAGTGAATGAGAAAACTTACAAAGCCAACATGGATGTTGTTTCTAACGCTAGCTGTACTACCAATTGCCTTGCTCCCCTTGCCAAGGTGGTTCATGAAGAGTTTGGCATTGTTGAGGGATTAATGACTACTGTGCATGCAACAACAGCTACCCAAAAGACTGTTGATGGGCCATCAATGAAGGATTGGCGAGGAGGCCGTGGTGCTGGACAAAACATCATTCCTAGTTCAACTGGTGCTGCTAAGGCTGTAGGAAAAGTTCTGCCAGAATTGAATGGGAAGCTCACTGGAATGGCTTTCCGTGTCCCAACACCTAATGTCTCTGTTGTTGACCTGACTTGCCGGCTAGATAAAAGTGCTTCTTATGATGATGTGAAAGCAGCGATAAAGTATGCATCGGAGGGTCCACTTAAAGGCATTTTGGGTTACACAGACGAGGATGTTGTCTCAAATGATTTTGTTGGCGATTCCAGATCAAGCATATTTGATGCTAAAGCTGGTATAGGCCTGAGCAAATCATTCGTGAAGCTCGTCTCCTGGTATGATAACGAGTGGGGTTACAGCAACCGAGTATTGGACCTCATCGAGCACATGGCGTTGGTTGCATCCATCCATTGA

SEQ ID NO: 221 - protein, 1581 aa, Solanum lycopersicum
ATGGCGGGTAAGCAATTAGATGTCCCGAGGACAAGTGGTGGTAACTTGAAAGAGCAATTAGTGAGGAGAACTTTGCAGAATGTGAGGTCTCAAGGGCACATATATGTTGAGCTTAGAGAGGATGGGAAGAGGCTGATCTTTTTCTGCACGTTATGTCATTCTCCATGTTATAGCGATTCTGTTCTGTTTAATCATTTGAAAGGCAATCTCCATACTGAAATGCTTGCTGCTGCTAAAGCCACATTGTTGAAACCTAATCCTTGGCCCTTCAATGATGGTGTACTTTTCTTTAATGATCCGGAGCAAGATAAGCAAGATAAGCAATCACCTAATGTGAATGTTGGGAAAAGCAGACTGGTTGACACTTGTCTTGAAGATGAAAGTAGCGTTGCTATTGTTGAGTATGATGACAACTTGAGACATAATGAGGATACATATGTTTCCGAGTATGAGTACGGCCTGCTTGATAGTGAGTTAATTGGTAATGAAGAGAGTGATTACTTGGTTATTCCGGGTGTGTTGTGTAAAGACGAATTATCTGATCTAGAAGTGAAACATATTGGTATTGGAAAAATTGCCGCTCGTATTAGTGTACGTGGCATTGACTCAAAAAGTATTCGTAGAATATGGTGTGAATGGTTGGCAAAGAAGGATTCTGATGATATGGATACCTCTGTGGTTCCAGATCATGATTTTGCGGTTGTTACATTTCCTTATAACTATAATTTGGGTAGGTCCCCGTTACTTGATGACAGATTTTTGCTCCCTTCCAGTCCTTATTCAGAATCTGAGGAGACCAGTGTTACTGGAAAGAGGAAGAGGAAGTCATTTTCAGACCCCGAGGATTTCAGCGAGTCTCTTAGTAATCATTGTGATTCGTCTGGGGAAGAATCTCAATCAACAAATAACTCAAATATGAAACTGATCTTGGGAACATGTGATGATCAACTAGTGAGTTCAAGAATTATATCCAGCAAAACTATGAGGAGAGAGTTGAGGAAACAGCAGCGAGTGGCTTCCGAGAGAATGTGTGATATCTGTCAACAGAAAATGCTTCCTGGTAAGGATGTGGCAACACTTCTGAGCTGGAAATCTGGAAAACTTATGTGCAGCAGTAGAAATATGTCTGGGGCATTTCATTTGTTTCACGTATCTTGCCTCATTCATTGGATACTTCAATGTGAATTGCAAACCTCTGTGAAACCTGTTGACGAACCAAAAATGGAACCAAAAGCAAAACGAAGATCTAAGAAGAAGACTGGAACCAAACACAATGCAAAAGAGAAGGAAGATGAGACAAAATCAGCGAGACGGATCAACTCAGTGTTCTGTCCGGAATGCCAGGGCACTGGTATCTGCATCGAGGGAGATGAGCTGGAGAAGCCTCCTGTCTCACTTTCTGAGGTGTACAGGCTCAAAATTAAACTTAGTGATGCACGAAAAGCATGGATGAAGAATCCTGAGGTGTTGCAAAATTGCTCAACAGGTTTTGATTTACCCCCTGAGCATGACGATCTTCTCCAGGAATATGTATCACCACTCAAATTGCTGCACTTTTATCGCGCCAATGTTTCACACTGA

SEQ ID NO: 222 - protein, 2025 aa, Solanum lycopersicum
ATGGAGTCAAGGCATTCCATTCATCACGTACTGCTTCTAGTTTTGTTTGGTTTGTCTGCCCTGTGCCAATGCTCTGCTACTGGGGTTGAAGCAAACCAGACAGCAGTACTGCTTGTGAATGCATCCGAAGCATCAGCAAGGAGAATACCTGATACCCTTTTTGGTATATTCTTTGAGGAGATCAATCATGCTGGTGCTGGTGGATTGTGGGCTGAGCTTGTCAACAATAGAGGTTTTGAAGGTGGAGGCCCAAACGTACCTTCAAATATTGATCCTTGGTCTATCATTGGAGATGAGTCCAAAGTGATTGTATCAACAGACCGTTCATCATGCTTTGATCGGAATAAAATTGCAGTTCAAGTGCAGGTGCTCTGTGACCATACAGGTGCCAATATCTGTCCAGATGGAGGAGTTGGCATCTACAACCCGGGATTCTGGGGCATGAATATTGAGCAGGGAAAGAGTTACAAACTAGTGCTTTATGTTCGTTCCGAAGAATCAGTCAATGTATCTGTCGCTTTAACTGGTTCAAATGGATTGCAAAAGCTGGCTGCTGCCAACATTGTAGCTGCTGATGTTTCAAGTTGGACGAAGGTGGAAATTTTGTTAGAAGCAACAGGAACAGATCCCAATTCAAGACTGGAATTGAGATCATCTAAGAAAGGTGTTATTTGGTTTGATCAAGTCTCATTAATGCCTACTGACACATACAAGGGGCATGGCTTCAGGAAAGATCTCTTTGGAATGCTTAAGGATTTAAAGCCAGCATTCATTAGATTTCCAGGTGGATGTTTTGTTGAAGGTGACTGGTTAAGAAATGCATTTCGGTGGAAAGAAACTATTGGACAATGGGAAGAGAGGCCCGGACATTTCGGTGATGTTTGGAATTACTGGACTGATGATGGACTTGGACATTTTGAGTTCCTGCAGCTTGCCGAAGACTTAGATTCACTGCCCGTGTGGGTTTTCAACAATGGAGTCAGTCACCATGATCAAGTTGACACTTCCAGTATTTTACCTTTTGTGCAAGAGATATTAGATGGTCTTGAGTTTGCAAGAGGTGATCCTACTTCAACATGGGGTTCTATTCGAGCCAAAATGGGACATCCAGAGCCTTTTGATTTGAGATATGTGGCTATCGGAAATGAGGATTGTGGAAAAACACAATACCGTGGAAATTACCTCAAGTTCTATACGGCCATCAAAGATAAATATCCAGATATTAAAATAATCTCAAACTGTGATGGTTCTACGAGACCACTGGATCACCCAGCTGATTTATATGATTTTCATATTTATAGCAGCGCAAGTTCTGTATTTTCTAATGCTCGCCATTTTGATAGTGCACCACGCAGAGGACCAAAGGCTTTTGTGAGTGAGTATGCCGTGACTGGAAATGATGCTGGAAAAGGTAGTCTTTTAGCAGCATTGGGTGAAGCTGGGTTCCTCATTGGGGTAGAAAAGAACAGCGAAGCAATTGAAATGGCAAGTTACGCACCCCTATTTGTTAATGACAATGACCGGAGGTGGAACCCAGATGCAATTGTCTTCACCTCTTCACAGATGTATGGAACGCCTAGTTATTGGATGCAGCACTTCTTCAAAGAGTCAAATGGCGCTACTCTTCTGAGTTCGTCATTACAAGCTAATCCTTCAAATTCACTTATAGCATCTGCCATCACTTGGCGAAATTCACTTGATAACAATGATTATTTGAGAATAAAGGTCGTGAACTTTGGAACCACTGCGGTTATAACTAAAATCTCTCTCACTGGATTGGGGCAGAACTCGTTAGAGACGTTATTTGGGGCTGTAATGACCGAGTTAACATCCAACAATGTGATGGATGAAAATTCCTTCAGAGAGCCTAATAAGGTAATACCTGTTAAAACACAAGTTGAGAAAGTTAGTGACAACATGGATGTTGTACTAGCTCCAAGATCTCTGAATTCAATTGACTTTTTATTAAGAAAATCAATAAACAACAATGTTGATACTGCTTCTGTCCTTAAATCTTCATGCTAA

SEQ ID NO: 223 - protein, 1266 aa, Solanum lycopersicum
AAAAAGTTAATCAAAGCCCTTGAATCTGCAAGAGGAAATGGTACCAGTATGATTTCTCTTATTATTCCTCCTGGTGATCAGATATCTAGGATTACCAAGATGTTGGCGGAAGAATATGGTACTGCATCAAATATTAAGAGCAGAGTGAATCGTCAGTCTGTCCTTGGTGCAATTACGTCTGCCCAGCAGAGGCTTAAACTGTATAATAAGGTACCTCCGAATGGTTTGGTCCTTTATACTGGAACTATAATGACTGAAGATGGGAAGGAAAAGAAGGTAACCTTTGACCTTACACCTTTTAAGCCCATTAATGCGTCTCTATACCTATGTGACAACAAGTTTCACACGGGACCTCTGGGTGAGCTTTTGGAATCAGATGAGAAGTTTGGTTTCATCGTCATGGATGGTAACGGTACTCTTTTTGGGACCTTAAGTGGCAACACTAGGGAAATCCTTCACAAATTTACTGTTGACCTTCCCAAGAAGCATGGAAGAGGAGGTCAATCAGCTCTGCGATTTGCTCGTCTTCGAATGGAGAAACGCCATAACTATGTGAGGAAGACAGCAGAGCTTGCTACTCAGTTCTTCATTAATCCAGCCACTAGTCAGCCAAATGTATCTGGACTAATACTTGCTGGGTCAGCTGATTTCAAGACGGAGCTGAGTCAGTCTGATATGTTTGATCCACGTCTTCAGGCAAAGATACTTAATGTGGTTGATGTGTCCTATGGTGGGGAAAATGGATTCAGTCAGGCCATCGAGCTATCTGCTGAGATTCTCTCGAATGTGAAGTTCATACAAGAGAAGCGCTTGATAGGAAAATTCTTTGAGGAGATCAGTCAAGATACTGGAAAGTATGTATTTGGTGTGGATGACACAATAAAAGCTCTGGAGATGGGAGCTGTTGAAACTCTTGTCGTTTGGGAAAATCTTGATATAAACCGTTATGTGCTGAAAAATAGTGTTACCAATGAGATTGTCATTAAGCACCTAAACAAGGACCAAGAGGCTGATCAAAGCAACTTCAAGGATTCTGAAAATTCAGCTGAATTGGAGGTCCAGGACAAAATGCCGCTATTGGAGTGGTTCGCCAATGAGTACAAAACCTTTGGTTGTTCACTTGAGTTTGTCACTAACAGGTCTCAAGAGGGGTCGCAGTTCTGCCGAGGATTTGGTGGCATTGGGGGCATCCTTCGTTACCAGCTTGACATGCGTTCGTTTGATGAGCCATCTGATGAAGGGGAATATTTTGAAGATTCTGATTAA

SEQ ID NO: 224 - protein, 1137 aa, Solanum lycopersicum
ATGGCAAAGAATTCATTTTTATTACACATTTTGTTGTTTTTTGTTCTTATTTTGAGTTTATGTGATGGGATTTTATCACAATATGAGTTGGAGGAACTTACATGGTTAGATGATAAAGATGATGAAATTTCAATGTTTCATAGCCAACATTCTTCTATGAGGAGATGTGATTTTACTTCTGGGAAATGGGTTTTTGATCAAAGTTATCCTCTATATGATTCAACTTGTCCTTATCTTAGTACTGCTGTTACTTGTACCAAAAATGGTAGACCTGATTCTGATTATGAAAAATGGAGGTGGAAACCTCATGGTTGTGAAATTCCAAGGTTTAATGCATTGGAATTTCTTGGAAGGATGAGGAAAAAGAGAATTATGCTTGTTGGTGATTCTATAATGAGGAATCAATGGGAATCTCTTGTTTGCTTAGTTCAATCTGTTATCCCTATGGCTAGAAAAACAGTAACTTATGTTGGTCCTACTATGGCCTTCCATGCTATGGATTTCGAGACAACAATAGAATTCTGCTGGGCTCCTTTCTTGGTGGAACTGAAGAAAGGACCAGAAAACAAGAGAATATTGCATCTGGACATGATTGAAGAGAATGCAAAGTATTGGAGAGGAGCTGATGTTCTTGTTTTTGATTCAGCTCATTGGTGGACTCACTCCGATAAATATAGTTCTTGGGACTTGATTATGGAAGGGAACAGTTTCTATAGAAACATGAACCCAATGGTTGCCTATGAGAAAGGACTAATGACATGGGCTAAGTGGGTTGATTTGAATCTTGATCCTCGAAAAACTCGAGTTTTCTTCAGAAGCATGTCACCTCGACATAACAGGGAAAATGGATGGAAATGCTTCAACCAGAGGGAGCCTCTGGAGTTCTTTAGCCATCCGCATGTACCAGAACCACTGTTGGTGCTAAAAGAGGTGCTACGAGGGATGAGTTTTCCGGTATCTTTCCAGGATATAACAACAATGACAGCTCTTCGAAGAGACGGGCATCCTTCTGTCTATAGTAAGTTTGTAAGCCAAACAGGTAAGCAGCACTTAGGAGACTATAAATCTGATTGTAGTCATTGGTGCCTTCCTGGAGTACCTGATACCTGGAACGAAATGCTGAATGTCATGCTCTAG

SEQ ID NO: 225 - protein, 1065 aa, Solanum lycopersicum
ATGTCTCTGCTTTCAGATCTTATCAACCTCAATCTCTCAGGTGATACTCAGAAGATCATTGCTGAATACATATGGATTGGTGGATCAGGCATGGACATGAGGAGCAAAGCCAGGACTCTCCCTGGTCCAGTTACTAGTCCTGCAGAACTACCCAAATGGAACTACGATGGATCGAGCACTGGTCAAGCTCCCGGAGAAGACAGTGAAGTGATCTTATATCCACAAGCAATCTTCAAGGACCCATTCAGAAGAGGCAACAACATCTTGGTCATGTGTGATGCCTATACTCCTGCTGGTGAGCCCATCCCAACAAACAAGAGGCACGCCGCCGCCAAGGTCTTCAGCCACCCTGATGTGGCTGCTGAGGAAACTTGGTATGGTATTGAACAAGAATATACCTTGCTGCAAAGGGAGGTCAACTGGCCTCTTGGATGGCCCATTGGCGGTTTTCCTGGCCCCCAGGGACCATACTACTGTGGAACCGGAGCTGACAAGGCCTTTGGACGTGACATTGTTGACGCCCATTACAAGGCTTGTCTCTATGCTGGGATTAACATCAGCGGGATCAATGGTGAAGTCATGCCGGGACAGTGGGAATTTCAAGTTGGACCTTCTGTTGGCATCTCAGCTGGTGATGAAGTGTGGGTAGCTCGTTACATTCTAGAGAGGATTGCAGAGATTGCTGGGGTGGTCGTGTCATTCGACCCCAAGCCTATTCCGGGCGACTGGAATGGTGCAGGTGCTCACACAAATTACAGCACCAAGTCGATGAGGGAAGACGGAGGCTATGAAATAATCTTAAAGGCTATTGAGAAGCTTGGCTTGAAGCACAAAGAACACATAGCTGCATATGGTGAAGGCAACGAGCGTCGTCTCACTGGAAAGCACGAAACAGCCAACATCAACACATTCAAATGGGGGGTTGCAAACCGTGGTGCATCTGTCCGTGTTGGAAGAGACACAGAGAAGGCAGGCAAGGGATACTTTGAGGACAGAAGGCCAGCCTCAAATATGGACCCATACGTCGTTACCTCCATGATTGCAGAAACCACCATCATCGGTTAA

SEQ ID NO: 226 - protein, 504 aa, Solanum lycopersicum
AAGATGATCCAAGAAAATATTGTGATATTATTATCCTTATTATTAATTGTGCCAATAAGTTGGGCAATTAATAATCCATTAATAGAACTTTCAAGTAGAGAAGAATTGGCTAAGATAGCTGGTTATGGTGAAGAAAAACTCTCTACTGTTTTCCTACATGGTACTCTTCTTTGTGGTCACACACCACATCCAGTTCCAGGTGCATCTGTGGCGGTGTATTGTGGAACTAGTGGGCCGGGTCGGGTAAGGAAATCATCATGGGCCAAAAATGTAACAAATGAAAGTGGAGATTTTATAATAGATGTCCCTTCTCATCTCCATGCTAATATTCATGATCCAAATCTATGTCACATTCAAATTCTTCACCTCCCAAAGGACTACTCACTTTGTCATCATCATTCTACAAAAAGGAAAACATTCACACTGACATCGTCCAATATTGGCGTTCGCGTTTACACAACTCCAACAATTTACCTGACCCCTAAAGCTAGCTCCCGAGTATAA

SEQ ID NO: 227 - protein, 648 aa, Solanum lycopersicum
ATGTTAGCGCATTGCACAAAGTCATCGCTGCCGGCGGCGGACGCCCACCTACCAACGCTGCGGCTCCCAAATCCACGGCGGAGTCCGGCCCCGGAGCTTCATTTCGGCTTCAATAGTTTTTCTACTAGAACATCGAAACTGCAAGTCAAAGCTTTGGACCGGAACAGTAACAACAGTAGCACCGGTGACGGCGTCGGCGCTAGTAAGAACGCCGCTTCATCGCCGTCATCATCGGTTCCGAACACGAACTACGTGGTGCCACTAGACAAGGCTTCGTCTTGCATTACTCGTCCCTTGGCCGAGATCCTACGTGACCTGAACAAGAGAATTCCTGATAACATCATCAAAGCTGGTAACAATGATGATCACTCCACCTTTATCACCTGGTACCATGCTAACCGGATGTTGAGCTTCTACGCTCCAGGCTGGTGCGGGGAAGTACGAGATGTCATTTTCTCAGATAATGGAAGTGTGACAGTTGTTTATCGTGTCACTATACGCGGATCAGATGGAGAGGCACACCGTGAATCAACTGGAACAGTATCACCAGGCAATGGACAGTACACAGATGTGGTTGCTGCTGCTGAGGAGATAGCATTTTGCAGAGCATGTGCTCGTTTCGGGCTTGGCTTGTATCTGTATCATTAA

SEQ ID NO: 228 - protein, 759 aa, Solanum lycopersicum
ATGGCTTCTTCTTCTGCAAAAATTCTACTTCCCCTCTCTCTCCTCTTCACCCTTTTATCTCTCTCCCAATCCACTAACCCTAATTTCATTTTAACCCTAGTTAACAACTGTCCTTACACCATCTGGCCCGCAATTCAACCCAATGCCGGTCACCCTGTTCTCGAACGAGGTGGATTTACCCTTCACTCACTTACCCACCGCTCTTTCCCCGCTCCCAACGCTCACTGGTCCGGCCGAATCTGGGCTCGTACCGGCTGCAATTACCAGCACGGAAAATTTTACTGCGCCACCGGAGACTGCGGCGGACGAATCGAGTGCGATGGACTCGGTGGTGCCGCCCCAGCTACATTAGCCCAGTTTGTTCTACATCACGGACATGCCGATTTTTCTACTTACGGTGTTAGTCTAGTGGATGGTTTCAACATTCCTCTTACTGTAACTCCACACGAAGGTAAAGGTGTTTGTCCAGTTGTTGGATGCCGGGCGAATTTGTTAGAGTCCTGCCCAGCAGTTTTGCAGTTTAGGTCACATGGTGGTCATGGACCTGTGGTTGGTTGTAAAAGTGCTTGTGAAGCATTTAAATCGGATGAGTTTTGTTGTAGGAATCATTATAATAGTCCACAGACCTGTAAACCATCGAGTTATTCCCAGTTCTTTAAGCATGCTTGTCCTGCTACATTCACATATGCACATGACAGTCCTTCACTTATGCATGAATGTTCGTCTCCGCGTGAACTAAAGGTCATTTTCTGCCATTAA

SEQ ID NO: 229 - protein, 1485 aa, Solanum lycopersicum
ATGATCGGTTGGTTTAAGCAGTTACATACTGCTTTTGGGTTTTCCTTTCTATGGCTTGTTTGCCTCATTTACTTCACACAGGGGTTCAGATCTTTTGTCTGGACTGCAGTTTCATATCAGTTGAAAGACAACTTGAAGCTATCACCATCAGCCTCCCAATTTGTCACTTCAATAGCATTCTTTCCGTGGAGCATAAAACCTTTATATGGGATATTATCTGATTGCTTTCCAATTAGAGGAAGGAAAAGAGTCCCCTACCTTATGCTGGCAACTTTGCTTTCGCTGTTGCCATGGCTCATTTTAGGGTTAAGTGATTTCCTGAGAAATACGAGGACAGAACTCATGATTCTTTTGACACTGCAGAACCTGGGTTCTGCAATGGCTGATGTTGTAATTGACGCAATGATCGCTGAGGCAGTAAGATTTGAGAGGGCATCATTTGCTGGTGATCTTCAGTCAGTATCTTGGCTGTCTATGGCATTCGGAGGAATCTGTGGTAGTTTGCTGGGAGGATATGCATTAGACAATATACGGATAGATATGATTTTCCTCCTTTTCTCTGTTTTACCTGCTTTACAGCTCTTTTCGTGTGGTTTGGTTGAGGAGGGTTCTGTACAAGGTAAAGCATTACCCGAAGGTTCTGCATTAAATGGTTCTGATATGACGAGTGGAATTGTTGATGAAGAAAATAAATTCTCTGGTGAAAAATCAAAAGTTAGTACTTTGAGGAGAAAGAAGAGCCAGAATAATACAAAAAGAGTGCCAACAACGGAAAGTAAATTTCAGGCTCCTGACAAAAAGGGATCATCATTGTCTCCGTGGTATCAATCACTGAAAATGGCCACTTCCACATTATTTAGGGCGTTCCGCCAGCCAGCCATTTTGAGACCAATGGCTTGGTTCTTTTTGGCTCATGTGACAGTTCCAAATCTCTCGACGGTTATGTTCTATTATCAGACAGAGTTCTTAAAACTGGAGGCATCTTTCCTTGGAACAGCACGTGTTCTTGGATGGTTGAGTCTCATGATCGGGACCTTGACTTACAATCGCTATTTAAAAAGGATGAGGTTGCGGCATATTCTCATGTTTACTCAAGTTTGCTTGGCCATTCTGACCATTCTAGATATGGTTTTGGTTTCTCGAGCTAATGTTTCATTAGGTATCTCAGACAAGGTTACAGTGCTATTTGGTTCTGCCCTTTCTGATGGCATCAATCAATTCAAGTTCATGCCCTTTCTTATCTTATCGGGGCAGCTTTGTCCTCCAGGGGTTGAAGGAACTTTATTTGCATTGTTCATGTCAATAAACAACTTAGGTTCTACGGTGGGATCCTTTGTAGGAGCAGGGTTGGCTTCAGTTCTAAACATCTCCTCAGGATCTTTTGACAATCTCTTCTTAGGCATTGGTATACAGGTTCTCTGCACTTTCATCCCAGTGGCCTTCCTTTTCTTGATTCCAAAGGAAGCTACAGGAATATCAGCATAG

SEQ ID NO: 230 - protein, 2001 aa, Solanum lycopersicum
ATGGCACAGATTCAGGTTCAGCATCAAACACCAGTGGCCGGAGGAAATGGTGTGGCGGTTGCTGCTGCTGCAGCCGGTGCTGCCGCTGCTGTAGCGGCACCAGGTGGAGGAGCCACGAGTCAGTTTACGTCCACGTCGTTGTATGTTGGAGACTTGGACTTTAACATCACTGATTCACAGCTCTATGATCTGTTCAACCATGTCGGTCAGGTTGTTTCTGTTAGGGTTTGTAGGGATCTTAGTACTCGGAGATCTCTTGGATATGGATATGTTAACTACAGTAACCCTTCCGATGCTGCAAGGGCAATGGAGTTGTTGAACTTCACTCCTGTGAATGGGAAGTCCATTAGGGTGATGTATTCTCATAGGGATCCCACTCTTCGTAAGAGTGGATCAGCAAATATATTTATTAAGAATTTGGACAAGTCGATTGACAACAAAGCATTACATGACACCTTTTCAAGCTTTGGCAACATTCTTTCTTGCAAGATAGCTACTGATTCTAATGGTCAGTCAAAGGGTTATGGTTTTGTACAATTTGACAACGAAGAATCTGCTCAAGGTGCCATAGATAAGTTAAATGGTATGTTGATGAATGATAAGCAAGTGTATGTTGGACACTTTCTTCGCAAACAGGAAAGAGAATCTACAACTGGCATGACAAAATTCAACAATGTCTATGTGAAAAATTTGGCTGAATCTGCTACCGATGATGAGTTGAAGAATGTTTTTGGTGAGTTTGGGACTATTACTAGTGCAGTAGTGATGAGGGATGCAGATGGAAAATCCAAGGGCTTTGGGTTTGTCAATTTTGAGAATGCAGATGATGCTGCTAAGGCTGTTGAAGCATTAAATGGAAAGAAATTTGACGAAAAAGAATGGTATGTTGGGAAAGCACAGAAAAAATCTGAAAGAGAGCAAGAATTGAAAAACAAGTTTGAGCAAACTGCCAAGGAGGCTGTTGATAAATACCAAGGTGTTAATTTGTATGTAAAAAATTTGGATGACACCATTGATGATGAAAAGCTCAAGGAACTTTTTGCCGAGTTTGGTACTATTACCTCATGCAAGGTTATGCGGGATCCAAGTGGAATCAGCAGGGGGTCTGGGTTTGTTGCTTTCTCTACTCCTGAAGAAGCTTCCAGAGCTCTTTCTGAGATGAACGGCAAAATGATAGTGAGCAAGCCACTCTATGTTGCACTAGCACAGCGGAAAGAGGAGAGAAGGGCAAAGTTGCAGGCACAATTTTCACAACTGCGACCAGTGGCAATGCCTCCATCACTTGCTCCTCGCATGCCAATTTACCCTCCTGGTGCTCCTGGGATTGGACAGCAACTATTTTATGGGCAAGGACCCCCTGCTCTAATTCCTCCCCAGGCTGGGTTTGGCTATCAACAACAGCTTGTTCCTGGAATGCGCCCTGGAGGAGCCCCTATGCCAAACTTCTTCATGCCGATGGTCCAGCAAGGACAACAGGGTCAGCGCCCAGGTGGTCGACGAGGAGCAGGACCTGTGCAACAATCTCAACAGCCTATGCCCTTGATGCAGCAGCAGATGATGCCAAGAGGAAGAATGTACCGCTATCCACCTGGACGCAATGTGCCAGATGGGCCAATGCCAGGTGTTGCTGGTGGTATGCTCTCTGTTCCATATGATATGGGTGGTATGCTTCCTCGTGATGCTGCTGCTATGGGACAGCCTGTGCCGATCTCTACATTGGCTACTGCCCTTGCAAATGCTCCACCTGAGCAGCAGAGGACGATGTTGGGTGAAAGTTTGTACCCACTTGTTGATCAGCTAGAGCACGAGCATGCTGCCAAGGTTACGGGCATGCTCCTTGAAATGGACCAGACCGAGGTTTTGCATCTGCTTGAATCACCGGAGGCTTTGAAGGCTAAAGTTTCCGAGGCTATGGATGTGCTGAGGAATGTTCAGCAGGCAACCAGCAGCCCGGCTGACCAACTTGCATCTCTTTCCCTTAATGACAACCTTGTTTCTTAG

SEQ ID NO: 231 - protein, 1458 aa, Solanum lycopersicum
ATGGGTACTGAGAGTAATTACCAGATGAAGGTGGTGAAAGGAGATCATGGTTATGTTCTTGAAGATGTTCCTCATTTGACTGATTATATCCCTGATCTTCCTACTCATGACAATCCATTGCGGTCCAATCCTGCATATTCAGTTGTGAAGCAGTACTTTGTTGACATGGATGATACTGTCCCGCAAAAGGTTGTTGTTCACAAGGACAGTCCCAGAGGGGTGCATTTCCGACGTGCTGGTCCACGTCAGAAGGTGTATTTCAATTCGGATGATGTTCGTGCTTGTATTGTAACTTGTGGTGGTTTGTGCCCTGGGCTAAACACAGTGATCAGAGAGATTGTACATAGCCTTGATTATATGTATGGGGTCAGCAAAGTCTTTGGGATCGATGGAGGCTACAGGGGTTTCTATTCCAAGAATATCATCAATTTGACACCAAAGACTGTTAATGACATTCATAAACGTGGTGGTACAATTCTTGGATCATCACGAGGAGGCCATGATACCACAAAGATTGTTGACAGCATACAGGACCGTGGAATTAATCAGGTTTATATAATTGGTGGTGATGGAACTCAGAAAGGAGCAGCTGTAATATACGAGGAAATCAGGCGGCGTGATCTCAAAGTAATTGTTGCTGGAATCCCAAAGACAATTGATAATGATATCCCTGTTATCGACAAGTCATTTGGTTTTGATACTGCTGTAGAGGAGGCTCAACGTGCCATAAATGCAGCTCATGTTGAAGCTGAAAGTGCTGAAAATGGTATTGGTGTGGTGAAGCTAATGGGACGCTATAGTGGATTCATCGCAATGTATGCCACTTTGGCGAGCAGAGATGTTGATCTCTGTCTAATTCCAGAGTCTCCATTTTATCTTGAAGGAGATGGCGGACTCTTCGAATACATTGAAAAACGGCTCAAAGAAAATGGGCACATGGTTATTGTGATAGCTGAAGGAGCAGGGCAAGAACTTCTTGCAGAAGAGAATGCGCATGCCAAAAACGAGCAAGATGCTTCGGGTAACAAGCTTCTTCAGGATGTTGGTTTGTGGATTTCCCAGAAAATCAGGGATCATTTTGCTAAAAAAACTAAGATGCCCATTACTCTTAAATATATAGATCCGACTTACATGATTCGTGCTGTTCCAAGTAATGCCTCTGATAATGTATATTGCACTCTTCTTGCTCAAAGTTGTGTTCATGGAGCAATGGCAGGATACACAGGTTTCACCTCAGGACTTGTCAATGGTCGCCAGACTTATATTCCATTCAATCGTATTACCGAGAAACAAAATATGGTGGTTATAACTGACAGGATGTGGGCACGTCTTCTTTCGTCAACCAATCAGCCAAGCTTCTTGCGCGTGAAAGACATTGAAGAGATTAAAAAGGAGGAGCAGCCGCAAACTCAATTATTGGACGGGGATAACAATGTACATGAGAACTCAGGTCACTGA

SEQ ID NO: 232 - protein, 870 aa, Solanum lycopersicum
ATGGGGAATGCGAACGCCAGAGAAGACGGCGCCGCCGGAGACGGCGACGGCGACGGTCAGGTATCGGGAAGAAGATCTAATGTTGAATCTGGAATAGTAGAAGATCACCACGCTCTGAACTCGCGAGTGCCTTCGGCTGACTTGATGGTCAATTCTCCTCCGCAGAGTCCACATCGTTCAGCTTCACCTCTCTTGTTCGGACCTCAGGTGCCAGTAGTTCCTTTACAAGGAGGGGATGGCAATCCTGTTAGCAATCAAATGTGGGGCAATGAATGCGAGGATGCCTCTGATCATTCACTAGAGGGCGGCATACCTACTTTGATAACATGGAGTTATGGTGGTAATAACGTTGCTATCCAAGGATCTTGGGACAACTGGACATCAAGGAAAATTCTCCAAAGATCAGGCAAGGACTATACCGTTCTTTTGGTCCTTCCATCGGGTATATATCATTACAAATTCATTGTGGATGGGGAAGTTAGATATATTCCAGAACTTCCATGTGTAGCAGATGAGACAGGCGTTGTCTTTAATCTTCTTGATGTTAATGACAATGTTCCAGAGAACCTCGAAAGTGTTGCAGAGTTTGAGGCCCCACCATCACCTGACTCTAGCTATGCGCAAGCTTTGATGGGAGAGGAGGATTTTGAAAAGGAGCCAGTGGCAGTTCCACCGCAACTTCATCTAACTGTTCTTGGTTCTGAAAACTCAGAAGAAGCACCTTCTTCTCCAAAACCCCAGCACGTAGTACTTAATCACCTCTTTATAGAGAAAGGATGGGCTTCTCAATCCGTTGTTGCTCTTGGTTTAACGCATAGATTCCAGTCCAAATATGTCACAGTTGTTCTCTACAAGCCACTGAAGAGGTGA

SEQ ID NO: 233 - protein, 1797 aa, Solanum lycopersicum
ATGTGCACAAAATTCGTGAAGGAAATGGAGAAGATGGCGTTGTACGTCGTCGTTTTGCTAATCATCTGCTATGCATCGCCGGCGAGATCAGACGGTTCCGATCATAAGTATAAATCCGGAGATCAGGTTCCACTTTATGCTAACAAAGTCGGTCCGTTCCATAATCCGAGTGAAACATACCGCTTCTTTGACCTTCCTTTTTGTACTCCAGATCATGTAAAAGAGAAGAAGGAAGCCCTTGGTGAGGTCTTGAATGGTGACCGATTGGTCAGTGCCCCTTACAAGCTTGACTTTTTGTATGATAAGGACTCAGAAATAGTTTGCAAGAAGAAGTTGTCCAAGGAAGAAGTTTCTCAATTCCGCAGCGCTGTTGCTAAAGACTACTACTTCCAAATGTATTACGATGACCTGCCCATTTGGGGTTTCTTAGGGAAAGTTGATAAGGAAGGAAAGTCTGACCCCAGCGAGTACAAATACTACTTGTTTAAACATCTTCATTTTGAGATCTTTTACAATGATGATCGTGTGATTGAGATCAATGCACGAACTGATCCTAATGCCCTGGTAGATATTACAGAGGATAAGGAGGTTGATGTTGACTTCATGTATACTGTAAAATGGAAAGAGACAAAAACACCTTTTGATAAGAGGATGGAGAAGTACTCACAGTCTTCCTCGTTGCCGCATCACTTGGAAATCCATTGGTTCTCAATTATCAATTCTTGTGTGACAGTTCTTCTCTTAACTGGTTTTCTTGCCACAATTCTTATGCGGGTCCTTAAGAATGACTTTGTCAAGTATGCACATGATGAGGAGGCGGCTGATGACCAAGAAGAAACTGGGTGGAAATACATCCATGGTGATGTGTTTAGGTACCCAAGTCACAAATCCTTGATGGCTGCAGCACTTGGGAGTGGCACACAGTTATTCACATTGACCATCTTTATTTTCCTTCTCTCACTTGTTGGTGTTTTCTATCCATACAACCGTGGAGCTTTATTCACGGCACTGGTCGTCATATATGCTCTCACATCTGGTATTGCAGGCTATACTGCTGCCTCTTTCTATTGCCAGCTAGAAGGGACAAACTGGGTCAGGAATTTGATATTGACCGGAGCCTTGTTTTGTGGACCCCTGTTTCTCACGTTCTGTTTCCTTAACAGCGTAGCAATTGCATACAGTGCTACTGCTGCACTGCCTTTTGGAACCATTGTGGTCATCTTTCTCATATGGGCTCTTGTGACATCACCTTTACTTGTATTGGGAGGGATAGCTGGAAAGAACAGCAGGGCTGAGTTCCAAGCTCCTTGTCGAACCACCAAATACCCAAGAGAGATCCCACCATTACCTTGGTACCGCGGAACTCTACCTCAGATGGCAATGGCTGGGTTTTTGCCTTTCAGTGCCATATATATCGAACTTTACTATATATTTGCAAGTGTTTGGGGTCACAGAATTTACACCATATACAGCATCTTATTTATAGTCTTCATCATTCTTATAATTGTGACTGCTTTTATTACCGTGGCCTTGACATACTTTCAACTTGCTGCTGAAGATCATGAATGGTGGTGGAGGGCTTTCCTTTGTGGTGGATCAACTGGACTGTTCATCTACGGATACTGCTTGTATTACTATTATGCACGTTCCGATATGACAGGGTTCATGCAAACCTCATTCTTCTTCGGGTACATGGCTTGCATCTGCTATGGTTTCTTTCTCATGCTCGGGTCTGTTGGTTTCCGTGCAGCCCTATTCTTTGTCCGTCACATATATCGATCGATCAAATGCGAGTAA

SEQ ID NO: 234 - protein, 1801 aa, Solanum lycopersicum
ATGTGCACAAAATTCGTGAAGGAAATGGAGAAGATGGCGTTGTACGTCGTCGTTTTGCTAATCATCTGCTATGCATCGCCGGCGAGATCAGACGGTTCCGATCATAAGTATAAATCCGGAGATCAGGTTCCACTTTATGCTAACAAAGTCGGTCCGTTCCATAATCCGAGTGAAACATACCGCTTCTTTGACCTTCCTTTTTGTACTCCAGATCATGTAAAAGAGAAGAAGGAAGCCCTTGGTGAGGTCTTGAATGGTGACCGATTGGTCAGTGCCCCTTACAAGCTTGACTTTTTGTATGATAAGGACTCAGAAATAGTTTGCAAGAAGAAGTTGTCCAAGGAAGAAGTTTCTCAATTCCGCAGCGCTGTTGCTAAAGACTACTACTTCCAAATGTATTACGATGACCTGCCCATTTGGGGTTTCTTAGGGAAAGTTGATAAGGAAGGAAAGTCTGACCCCAGCGAGTACAAATACTACTTGTTTAAACATCTTCATTTTGAGATCTTTTACAATGATGATCGTGTGATTGAGATCAATGCACGAACTGATCCTAATGCCCTGGTAGATATTACAGAGGATAAGGAGGTTGATGTTGACTTCATGTATACTGTAAAATGGAAAGAGACAAAAACACCTTTTGATAAGAGGATGGAGAAGTACTCACAGTCTTCCTCGTTGCCGCATCACTTGGAAATCCATTGGTTCTCAATTATCAATTCTTGTGTGACAGTTCTTCTCTTAACTGGTTTTCTTGCCACAATTCTTATGCGGGTCCTTAAGAATGACTTTGTCAAGTATGCACATGATGAGGAGGCGGCTGATGACCAAGAAGAAACTGGGTGGAAATACATCCATGGTGATGTGTTTAGGTACCCAAGTCACAAATCCTTGATGGCTGCAGCACTTGGGAGTGGCACACAGTTATTCACATTGACCATCTTTATTTTCCTTCTCTCACTTGTTGGTGTTTTCTATCCATACAACCGTGGAGCTTTATTCACGGCACTGGTCGTCATATATGCTCTCACATCTGGTATTGCAGGCTATACTGCTGCCTCTTTCTATTGCCAGCTAGAAGGGACAAACTGGGTCAGGAATTTGATATTGACCGGAGCCTTGTTTTGTGGACCCCTGTTTCTCACGTTCTGTTTCCTTAACAGCGTAGCAATTGCATACAGTGCTACTGCTGCACTGCCTTTTGGAACCATTGTGGTCATCTTTCTCATATGGGCTCTTGTGACATCACCTTTACTTGTATTGGGAGGGATAGCTGGAAAGAACAGCAGGGCTGAGTTCCAAGCTCCTTGTCGAACCACCAAATACCCAAGAGAGATCCCACCATTACCTTGGTACCGCGGAACTCTACCTCAGATGGCAATGGCTGGGTTTTTGCCTTTCAGTGCCATATATATCGAACTTTACTATATATTTGCAAGTGTTTGGGGTCACAGAATTTACACCATATACAGCATCTTATTTATAGTCTTCATCATTCTTATAATTGTGACTGCTTTTATTACCGTGGCCTTGACATACTTTCAACTTGCTGCTGAAGATCATGAATGGTGGTGGAGGGCTGTCATTCGTTTGTGGTGGATCAACCGGCTTGTTCATCTATGGCTACTGCCTGTATTACTATTATGCCCGATCAGACATGTCAGGTTTCATGCAAACCTCATTCTTCTTTGGTTACATGGCTTGCATTTGCTATGCCTTCTTTCTCATGCTTGGAACTGTTGGTTTCCGAGCTGCTTTGTTTTTCGTCCGCCACATATACCGTTCAATCAAGTGCGAGTAA

SEQ ID NO: 235 - protein, 1458 aa, Solanum lycopersicum
ATGGCTCTACTCGTTGAGAAGACCACCTCTGGCCGCGAGTACAAGGTGAAGGACATGTCCCAGGCTGACTTCGGCAGGCTCGAAATCGAGCTTGCCGAAGTTGAAATGCCTGGTCTCATGGCTTCCCGTGCTGAATTTGGCCCTTCACAGCCCTTTAAAGGTGCAAAGATCACTGGATCTCTGCATATGACTATCCAAACTGCTGTTCTTATTGAAACCCTAACTGCTTTGGGTGCTGAAGTTAGATGGTGTTCTTGCAACATCTTCTCAACTCAGGACCATGCTGCAGCAGCCATTGCACGTGACAGTGCTGCTGTTTTTGCCTGGAAAGGTGAGACTTTGCAGGAGTACTGGTGGTGTACTGAGAGGGCACTTGATTGGGGTCCAGGTGGTGGTCCTGATCTGATTGTTGATGATGGAGGTGATGCTACTCTGTTGATTCATGAGGGTGTTAAGGCTGAAGAGGAGTTTGCTAAGAATGGAACAATCCCAGATCCTAGTTCTACTGATAATGTTGAGTTTCAACTTGTGCTTACTATTATTAAGGAGAGTTTAAAGACTGATCCTTTAAAATACACTAAGATGAAGGAGAGACTTGTTGGTGTTTCTGAGGAAACTACCACTGGTGTTAAGAGGCTTTACCAAATGCAGGCTAATGGAACTTTGCTTTTCCCTGCTATTAATGTTAATGACTCTGTTACCAAGAGCAAGTTTGACAACTTGTACGGATGCCGCCACTCACTTCCCGATGGTCTCATGAGGGCTACTGATGTTATGATTGCTGGAAAGGTTGCCCTTGTTGCTGGTTATGGAGATGTCGGCAAGGGATGTGCTGCTGCCATGAAACAAGCTGGTGCACGCGTTATTGTGACTGAGATTGATCCAATCTGTGCTCTCCAGGCTACCATGGAAGGTCTCCAGGTTCTTCCTCTTGAGGATGTTGTTTCTGAGGTTGATATCTTTGTGACCACCACTGGTAACAAGGACATCATCATGGTTGACCACATGAGGAAGATGAAGAACAATGCCATCGTCTGCAACATTGGTCACTTTGACAATGAAATCGACATGCATGGTCTTGAGACCTTCCCTGGTGTGAAGAGGATCACAATCAAGCCTCAAACCGACAGATGGGTTTTCCCAGACACCAACAGTGGCATCATTGTGTTGGCCGAGGGTCGTCTCATGAACTTGGGATGTGCCACAGGACACCCCAGTTTTGTGATGTCTTGCTCTTTCACTAACCAAGTCATTGCCCAACTCGAGTTGTGGAATGAGAAGAGCAGTGGTAAATACGAGAAGAAGGTATACGTTTTGCCAAAGCACCTTGACGAGAAGGTTGCTGCCCTTCATCTTGGAAAGCTCGGAGCCAAACTTACCAAACTTACCAAGGATCAAGCTGACTACATTAGCGTACCAGTTGAGGGTCCTTACAAGCCTGCTCACTACAGGTACTGA

SEQ ID NO: 236 - protein, 372 aa, Solanum lycopersicum
ATGGCAGCATATGCAGCAATGAAGCCAACCAAGCCTGGGCTTGAGGAGCCTGCAGAGATGATTCACAAGATTAGGATCACTCTCTCCTCCAAGAACGTTAAGAATCTCGAGAAAGTGTGCGCTGATTTGGTTCGTGGTGCCAAGGATAAGAGGCTCAGGGTGAAGGGACCAGTGAGAATGCCCACAAAGGTCCTTAACATCACCACTAGAAAGTCTCCTTGTGGAGAAGGTACAAATACATGGGACAGATTTGAGCTTCGTGTCCACAAGCGGGTGATTGACCTTTTCAGCTCACCAGATGTTGTGAAACAAATCACTTCAATCACCATTGAACCCGGTGTTGAGGTTGAGGTCACTATTGCGGAATCTTAG

SEQ ID NO: 237 - protein, 1017 aa, Solanum lycopersicum
ATGGCAAACGGAAAGATCAAAATCGGAATCAACGGATTCGGTAGAATTGGTCGTTTGGTGGCTAGAGTTGCTCTACAGAGAGATGATGTTGAACTAGTTGCAGTGAATGATCCATTTATTTCCACTGATTACATGACATATATGTTTAAGTATGATTCAGTACATGGACAATGGAAGCATCATGAGCTAAAGGTCAAGGATGAGAAGACACTTCTCTTTGGAGAGAAGGCTGTTACAGTTTTTGGAATCAGGAACCCTGAAGATATCCCATGGGGTGAAGCTGGTGCTGACTTCGTTGTTGAATCAACCGGTGTCTTCACTGACAAGGACAAGGCTGCTGCTCACTTGAAGGGTGGTGCCAAGAAGGTTGTGATCTCTGCTCCTAGCAAAGATGCTCCCATGTTTGTTGTGGGTGTCAACGAGAATGAATACAAGCCAGAGCTGGACATTGTCTCCAATGCTAGTTGCACAACGAACTGCCTTGCACCTTTGGCTAAGGTTATCAATGATAGGTTTGGCATTGTTGAGGGTCTCATGACCACTGTCCACGCCATGACTGCCACCCAGAAAACTGTTGATGGTCCATCCATGAAGGACTGGAGAGGTGGAAGAGCTGCTTCATTCAACATCATCCCTAGCAGCACTGGTGCAGCCAAGGCTGTTGGAAAAGTGCTCCCACAACTTAACGGCAAATTGACTGGAATGGCCTTCAGAGTACCAACTGCTGATGTCTCCGTTGTCGATCTTACTGTAAGACTCGAGAAAGAAGCCTCCTATGAAGACATTAAGGCTGCAATCAAGGAGGAATCAGAGGGTAAATTGAAGGGTATCTTGGGATACACTGAAGATGATGTGGTTTCCACAGACTTTGTTGGTGACAGCAGGTCAAGCATTTTTGATGCCAAGGCTGGAATTGCTTTGAGCAAGAATTTTGTGAAAGTTGTGTCATGGTATGACAACGAATGGGGTTACAGTTCCCGTGTGATTGATTTGATCTGCCATATGGCTAAGGCTTGA

SEQ ID NO: 238 - protein, 693 aa, Solanum lycopersicum
ATGGCTTCTAATTCAGCCTTTTCTCTGTTCTTGATTTTGTTAATTATTACACAATGTTTATCAGTCCTCAATGCTGCTAAAGATTTTGACTTTTTCTACTTTGTTCAACAGTGGCCAGGGTCATACTGTGACACAAAACAAAGTTGTTGTTACCCTACAACTGGAAAACCAGCAGCAGATTTTGGAATTCATGGACTTTGGCCTAATAATAATGATGGGACTTATCCATCAAATTGTGATCCAAACAGTCCTTATGACCAATCTCAGATTTCTGACTTAATTAGTAGTATGCAACAAAATTGGCCAACACTAGCGTGCCCAAGTGGTAGTGGCTCAACATTTTGGTCACATGAATGGGAAAAGCATGGCACTTGTGCTGAATCCGTTCTCACAAACCAACACGCTTATTTTAAGAAGGCTCTTGATCTCAAGAATCAAATTGATCTTTTGTCAATTCTTCAAGGTGCTGACATTCATCCTGACGGCGAATCTTATGACCTGGTCAATATTAGAAATGCAATTAAAAGCGCGATTGGATATACTCCTTGGATCCAATGTAATGTAGACCAGTCGGGTAACAGTCAGCTATACCAGGTTTACATTTGTGTTGATGGCTCGGGTTCAAGTCTCATTGAGTGCCCTATTTTCCCTGGAGGGAAATGTGGCACAAGCATTGAGTTCCCAACATTTTAA

SEQ ID NO: 239 - protein, 1464 aa, Solanum lycopersicum
ATGGTAGATACATTGACTGAATTATGCTCCAAACTTGCTGGACTGATGTTCATTTTGGGTACAATTCAGCAACTTGTCCCTCATTCTTTCTACAAGCGCATCAAAACATTGTGGCATCGAATAGAAAATTATTTCTACCCTTATGTCCAAATTACCATCGATGAATACTCCAACTATAAAAGAAATGAAGTTTACTCTCTTATAAAAGTTTATTTAGGTACTAAGTCCACTAACGATGCAAAGTATCTTAAAGCTGAAATGTTCAAGAAGAGCAAGTTTTTAGCTGTTAGCTTGGATGAAGGAGAAGAGGTAATTGATGAATTTCATGGTGTGAAGCTCAAATGGAACTCTTACACGGAAACATTCCCTGATAATTCTTCAGGGGGGCGTTCTCAACTTCCGATTGAAAAGAAATGTTATACGCTAACTTTCAATAAGAAAAATCAAGAGATGGTCACCGGACAATACTTGAAACATGTGATGGAAGAAGGGAAGGCCATTGAGTTCAAGAACAAGAAGCAGAAGATCTACTCTAACGAAGAAGGGGAGAGGTATTGGTATGGTAAAGGTATGTGGAGGAACATAAATTTCGAGCACCCTGCAACATTTGATACACTGGCTATGGATCCTACGAAGAAGGAAGAAATAATCAATGATCTCGTTGCTTTCAGCAAGGGGAAGGACTACTATTCCAAGGTTGGAAAGGCGTGGAAGCGCGGTTATCTTCTTTATGGTCCACCAGGGACTGGAAAATCAACTATGATTGCAGCAATTGCGAATTACTTGAACTATGATATTTATGATCTCGAGCTCACCTCAGTTAAGGATAACTCAGAGCTTAAGAAATTGCTCATGGAAACAACAAGCAAGTCCATCATTGTCATTGAAGATATCGATTGTTCTATTGATCTCACGGGCAAGAGAAAGAAGAAAAATAAAAAGGAAACAACGAATGAGGAGGAGGAAGAAGATTCAGACTCGACCACAGAGAAAAAGAACGACAAGAATGAATCTAGCAAACTTACTCTTTCGGGGCTTTTGAATTTCATAGATGGAATATGGTCAGCTTGTGGTCAAGAGAGAATTATTATATTTACGACTAATCATGTGGACAAACTTGATCCAGCTCTGATACGTAGAGGACGGATGGATATGCACATTGAGATGTCGTATTGCAAATATGAAGCATTCAAAGTGTTGGCTAAGAATTACTTGGGAATCGAAACACATCCTCTGTTTCAAGAGATTCAAAGTTTATTAGAGGAAGTAGACGTGAGTCCTTGTGATGTGGCTGAGAACTTGATGCCCAAAAATGCTTCAGGAAACCCTGAGATTTGCTTGGAAAGCTTAGTCAAAGTTTTGATAAAGGCCAAGAGAAAGGCCAGCTTGAATTCCCACAAGAACAAAAAATCCTCCACAAAACTAATCATAACTTATGGCCGTTTCAAAAAGCTGTTTCAATGA

SEQ ID NO: 240 - protein, 387 aa, Solanum lycopersicum
ATGCAGATCTTCGTGAAAACTTTGACGGGTAAAACCATAACCCTCGAGGTTGAATCCAGTGACACTATTGACAATGTCAAGGCCAAGATTCAAGACAAGGAAGGAATTCCTCCAGACCAGCAAAGGCTGATTTTTGCTGGTAAGCAGCTAGAAGATGGCCGCACTCTTGCTGACTATAACATTCAGAAAGAGTCGACTTTGCATTTGGTGCTGAGGCTTCGTGGAGGAATTATTGAGCCTTCTTTGATGGCTTTGGCTAGGAAGTACAATCAGGAAAAGATGATTTGCCGCAAGTGCTACGCTCGCCTACATCCCCGTGCTGTCAACTGCCGCAAGAAGAAGTGTGGGCACAGCAACCAGTTGAGGCCTAAGAAGAAGATCAAGTAG

SEQ ID NO: 241 - protein, 438 aa, Solanum lycopersicum
ATGCCGAAGAACAAGGGTAAGGGAGGTAAGAACAGGAAGAGAGGAAAGAATGAAGCCGATGATGAGAAAAGAGAGCTTGTTTTCAAAGAAGATGGACAGGAATATGCTCAGGTCCTTCGTATGCTTGGTAATGGACGATGTGAAGCTATGTGTATTGATGGAACTAAGCGTCTTTGTCACATACGTGGTAAGATGCATAAGAAAGTATGGATCGCTGCTGGTGATATCATCCTTGTTGGCCTACGTGACTATCAGGATGACAAAGCTGATGTGATCTTGAAGTACATGCCTGATGAGGCCAGGTTATTGAAGGCATATGGAGAGTTACCAGAGAACACAAGACTTAATGAGGGCATTGCTAATGGTCTTGATGAAGAGGATGAGGGTGCTGGTGATGATTACATTGAGTTTGAGGATGAAGACATTGATAAAATCTAA

SEQ ID NO: 242 - protein, 1605 aa, Solanum lycopersicum
ATGCAGATATTTGTTAAGACACTCACCGGAAAGACCATCACTCTTGAGGTTGAGAGTTCTGACACCATTGATAATGTCAAAGCTAAGATTCAAGACAAGGAAGGCATTCCTCCAGATCAGCAGAGACTGATCTTTGCTGGGAAACAGCTTGAAGATGGCCGAACACTTGCTGATTACAACATCCAAAAAGAGTCTACCCTTCATCTTGTCCTCCGTCTACGTGGTGGGATGCAAATCTTTGTTAAAACTCTGACCGGAAAGACTATAACTCTTGAGGTCGAGAGCTCAGACACCATCGATAATGTCAAAGCTAAAATTCAAGACAAGGAAGGTATTCCCCCAGACCAGCAGAGGCTGATCTTTGCTGGGAAACAGCTTGAAGATGGCCGAACACTTGCTGATTACAACATCCAAAAGGAGTCCACCCTTCACCTTGTCCTTCGCCTTCGTGGTGGTATGCAGATCTTTGTCAAGACACTTACTGGAAAGACCATCACCCTTGAAGTTGAAAGTTCCGATACTATTGACAATGTGAAGGCCAAAATTCAGGATAAGGAAGGGATTCCTCCAGACCAGCAGAGACTGATCTTTGCCGGCAAGCAACTTGAGGATGGAAGGACCCTGGCTGACTACAATATTCAGAAAGAGTCTACCTTACATCTTGTTCTTCGACTGAGGGGTGGCATGCAAATATTTGTCAAGACATTGACAGGGAAGACAATTACTTTGGAGGTTGAGAGTTCAGATACTATCGACAATGTTAAAGCAAAGATACAAGACAAGGAGGGTATTCCTCCAGACCAGCAGAGATTGATCTTTGCTGGAAAGCAACTTGAGGATGGAAGGACGTTGGCAGATTACAACATTCAGAAAGAATCAACCCTGCACCTTGTTCTTCGCCTTAGAGGTGGCATGCAAATTTTTGTCAAGACTTTGACTGGGAAGACAATTACATTGGAGGTTGAGAGTTCAGATACCATTGACAATGTGAAAGCAAAGATTCAAGACAAGGAGGGTATTCCCCCAGACCAACAGCGTTTGATCTTTGCTGGTAAACAACTTGAGGACGGGAGGACTCTTGCAGACTACAACATCCAGAAGGAGTCAACTCTCCATCTGGTGTTGCGTTTGAGAGGAGGGATGCAGATCTTTGTGAAGACTTTGACTGGGAAGACAATCACATTGGAGGTTGAGAGTTCAGATACCATTGACAATGTGAAAGCAAAGATTCAAGACAAGGAGGGTATTCCCCCAGACCAACAGCGTTTGATCTTTGCTGGTAAACAACTTGAGGATGGGAGGACTCTTGCAGACTACAACATCCAGAAGGAGTCAACTCTCCATCTGGTGTTGCGTTTGAGAGGAGGGATGCAGATCTTTGTGAAGACTCTTACTGGGAAGACAATCACATTGGAGGTGGAGAGCTCTGATACTATTGACAATGTGAAAGCAAAGATACAGGACAAGGAAGGGATCCCACCAGATCAACAGAGGCTCATCTTTGCTGGTAAGCAGCTTGAGGATGGCCGCACCCTTGCAGACTATAACATCCAGAAAGAGTCTACTCTTCATCTTGTCCTGAGGCTCCGTGGCGGTGACTTGTGA

SEQ ID NO: 243 - protein, 1374 aa, Solanum lycopersicum
ATGCAGATATTTGTTAAAACCCTCACTGGAAAGACTATCACCCTTGAGGTGGAAAGCTCAGACACCATTGACAATGTTAAAGCCAAGATCCAGGACAAGGAAGGCATTCCCCCAGACCAGCAGAGGCTGATCTTTGCAGGAAAGCAGCTTGAAGACGGTCGTACTCTAGCTGATTACAACATTCAGAAAGAGTCAACTCTCCACTTGGTGCTCCGTCTTCGTGGTGGCATGCAAATTTTCGTGAAAACTTTGACAGGAAAGACCATCACCCTTGAGGTTGAAAGCTCTGACACCATTGACAATGTTAAGGCTAAGATCCAAGACAAGGAAGGCATTCCCCCAGACCAGCAAAGGTTGATCTTTGCGGGCAAGCAGCTTGAGGACGGCAGGACCCTTGCTGATTACAACATCCAGAAGGAATCAACCCTTCACTTGGTGCTCCGTCTTCGTGGTGGAATGCAAATTTTTGTGAAGACTTTGACTGGAAAGACCATCACCCTTGAGGTTGAAAGCTCCGACACCATTGACAATGTTAAGGCTAAGATCCAAGACAAGGAAGGCATTCCCCCAGACCAGCAAAGGTTGATCTTTGCGGGCAAGCAGCTTGAGGACGGCAGGACCCTTGCTGATTACAACATCCAGAAGGAGTCAACCCTTCACTTGGTCCTTCGTCTTCGCGGTGGGATGCAAATCTTTGTGAAGACCTTGACTGGCAAGACAATCACCCTTGAGGTGGAAAGTTCTGACACCATTGACAATGTTAAGGCCAAAATTCAGGACAAAGAAGGTATTCCACCAGACCAGCAGAGGTTGATCTTTGCCGGAAAGCAACTTGAGGACGGCCGTACCCTTGCTGATTACAACATCCAGAAGGAGTCAACCCTTCACTTGGTCCTGCGTCTCCGAGGTGGAATGCAAATCTTCGTGAAGACCTTGACTGGCAAGACTATTACCCTTGAGGTGGAAAGCTCCGACACCATTGACAACGTAAAGGCCAAGATTCAGGACAAAGAGGGTATCCCACCAGACCAGCAAAGGTTGATTTTTGCTGGCAAGCAGTTGGAAGATGGAAGGACATTAGCCGATTACAATATTCAGAAGGAATCAACCCTTCATCTTGTGTTGAGGCTCAGGGGAGGTATGCAGATTTTTGTGAAGACCTTGACTGGGAAAACCATAACTTTGGAGGTGGAGAGTTCTGATACCATTGACAATGTGAAGGCTAAAATTCAGGACAAGGAGGGAATCCCACCGGACCAGCAGAGACTGATCTTTGCAGGGAAGCAACTTGAAGATGGAAGGACTCTGGCGGACTACAACATCCAAAAGGAGTCTACTTTGCACCTTGTCCTCCGTCTCCGTGGTGGTTTTTGA

SEQ ID NO: 244 - protein, 1182 aa, Solanum lycopersicum
ATGGAGACTTTCTTATTCACATCTGAATCAGTCAACGAGGGACATCCCGACAAGCTCTGTGACCAGGTCTCTGATGCAGTGCTCGATGCCTGTCTAGCTCAGGACCCTGAAAGCAAAGTTGCATGTGAGACTTGTACCAAGACCAACTTAGTTATGGTCTTTGGAGAGATCACCACCAAGGCCAATATTGATTATGAGAAGATTGTACGTGACACTTGCCGGGAAATTGGATTTGTGTCCCCTGATGTTGGTTTGGATGCTGACAATTGCAGAGTCCTTGTGAACATTGAGCAGCAGAGCCCTGATATTGCTCAAGGTGTTCATGGTCATTTGACTAAGCGACCTGAGGAGATTGGTGCTGGTGACCAGGGTCACATGTTTGGCTATGCCACTGACGAGACACCGGAGTTGATGCCCCTTAGCCATGTTCTTGCTACCAAACTTGGAGCTCGCCTTACTGAGGTCCGCAAGAATGGAACTTGCTCTTGGCTTAGACCTGATGGTAAAACACAAGTGACTGTTGAGTATCACAATGACAATGGTGCTATGGTTCCTCTACGTGTTCACACTGTTTTAATCTCCACTCAGCATGATGAGACTGTTACCAATGATGAAATTGCTCGTGATCTCAAAGAGCATGTCATCAAGCCTGTCATCCCCGAGAAGTATCTTGATGAGAACACCATTTTCCACCTCAACCCTTCAGGCCGTTTTGTCATTGGTGGACCTCACGGTGATGCTGGTCTCACTGGCCGTAAGATCATCATTGACACTTACGGAGGTTGGGGTGCTCACGGAGGTGGTGCTTTCTCTGGGAAGGATCCTACCAAGGTCGACAGAAGTGGAGCCTATATCGTGAGGCAAGCAGCTAAGAGCATTGTTGCTAACGGTCTTGCCAGAAGGTGCATTGTTCAGGTTTCATATGCCATCGGTGTGCCTGAGCCATTGTCCGTCTTTGTGGACACTTATGGAACTGGGAAGATCCCTGACAAGGAGATTCTCAACATTGTGAAGGAGAACTTTGATTTCAGGCCCGGAATGATTTCCATTAACCTCGATCTACTGAGGGGTGGCAATGGTCGATTCTTGAAGACTGCTGCCTATGGGCATTTTGGTAGAGATGATCCTGACTTCACATGGGAAGTAGTTAAGCCTCTCAAGTGGGACAAGCCTGAAGCTTAA

SEQ ID NO: 245 - protein, 600 aa, Solanum lycopersicum
ATGAATGATCCTTACAGATCTAAACCCAGTAGCGGGCGAACAAATTTTGCTTCATGTATTTGTGCCACTGTCTTCTTGATCTTTCTTATTGCAGGCATTGTTGTCGCTTACTTCTTCCTCTTCAAACCCAAAACTCCCAAAATCGCCGTTGAGGCAGTTCGATTCCCTACTTTCTCTGTTACCAACGGTACTGTTAACTTCACCTTCTTCCAGTACGTCTCCGTCACAAACCCAAATCGGGATGAGTTTACTCACTATGACAGTTCGCTTCAGCTCAGTTACTCAGGGGAGCCGGTGGGTCTTGTTTTTATTCCCGCCGGGAAGATCGACGGTGGTAGGACCCAGCATATGTCGGCGAAATTTGATGTTCAGTCTTACCCACTTCCAGCCAAGCTGAAAGCTGATGTCTCCGGCGGGATGATTCCGACGACGGCGGTGAACGTCTTTGATGAGGGACCCACAATGGAGGTGGAAACGAGGATGAAGTTGGTGGGAAGAGTTAGGGTATTGAAAGTGTTTACACATAGGGTGGACAGTGGAGTGAAATGTGGAGTAATGATTCAAGTCAGTAGTGGCTCTGTTTTAGGCGTCCGTTGCTGA

SEQ ID NO: 246 - protein, 1374 aa, Solanum lycopersicum; identical to SEQ ID NO: 243

SEQ ID NO: 247 - protein, 600 aa, Solanum lycopersicum; identical to SEQ ID NO: 245

(19) United States
(12) Patent Application Publication
(10) Pub. No.: US 2024/0417750 A1
(43) Pub. Date: Dec. 19, 2024

(54) RESISTANCE GENES AGAINST ROOT KNOT-NEMATODES IN TOMATO

(71) Applicant: UNIVERSITY OF TENNESSEE RESEARCH FOUNDATION, KNOXVILLE, TN (US)

(72) Inventor: TAREK ABDELFATTAH HEWEZI, KNOXVILLE, TN (US)

(21) Appl. No.: 18/694,018

(22) PCT Filed: Sep. 22, 2022
(86) PCT No.: PCT/US2022/076830

§ 371 Date: Mar. 21, 2024

Related U.S. Application Data
(60) Provisional application No. 63/246,814, filed on Sep. 22, 2021.

(51) Int. Cl. C12N 15/82; C12Q 1/6874; C12Q 1/6895

(52) U.S. Cl. CPC C12N 15/8285; C12N 15/8205; C12Q 1/6874; C12Q 1/6895; C12Q 2600/13; C12Q 2600/158

(57) ABSTRACT

The invention relates to genes which may be utilized to induce resistance to root-knot nematode (RKN). More specifically the present disclosure provides genes that, when inactivated, silenced, underexpressed, and/or overexpressed in a plant, particularly, a tomato plant, can confer upon the plant resistance to RKN. Methods of using these genes to obtain plants, particularly tomato plants, that are resistant to RKN are also provided.

### CROSS-REFERENCE TO RELATED APPLICATION

This application claims the benefit of U.S. Provisional Application Ser. No. 63/246,814, filed Sep. 22, 2021, the disclosure of which is hereby incorporated by reference in its entirety, including all figures, tables and amino acid or nucleic acid sequences.

### STATEMENT REGARDING SEQUENCE LISTING

The Sequence Listing for this application is labeled “Seq-List.xml” which was created on Jul. 29, 2022 and is 405,595 bytes. The entire content of the sequence listing is incorporated herein by reference in its entirety.

### BACKGROUND OF THE INVENTION

Root knot nematodes (RKN; Meloidogyne spp.) are very devastating plant pathogens, which are estimated to cause world-wide annual yield loss of more than $170 billion (Elling, 2013). The Meloidogyne species, M. arenaria, M. hapla, M. incognita, and M. javanica are considered the species of major economic significance (Elling, 2013; Jones et al., 2013). Among these species, M. incognita is inarguably the most devastating plant-parasitic nematode species. RKN are biotrophic, sedentary, obligate endoparasites. The second stage juveniles (J2) is the only infective stage of the RKN life cycle. The J2 penetrates the root tip epidermis at the elongation zone and migrates intercellularly searching for compatible cells to induce the formation of giant-cells as a permanent feeding site. Once the compatible cells, typically between three and seven, were identified, the infective J2 became sedentary and stimulates the selected cells to enlarge via recurrent mitosis without cytokinesis to form the giant-cells (Hewezi, 2020). The formation of giant-cells also stimulates surrounding cells to divide and enlarge in size, resulting in the formation of knot-like structures known as galls. Gall formation on plant roots is considered the hallmark characteristic of RKN infections. The sedentary juveniles feed from the giant-cells for an extended period of time and experience three molts, and finally develop into adult males or females, which lay hundreds of eggs embedded in a gelatinous matrix. RKN species of agricultural importance reproduce solely through mitotic parthenogenesis. Although parthenogenesis is generally believed to reduce genetic diversity, and hence nematode pathogenicity, RKN have maintained a high level of genetic variation that allows rapid adaptation to hostile environments and resistant hosts (Castagnone-Sereno, 2006). This may have contributed towards the extremely wide host range and geographic distribution of RKN. Indeed, RKN can infect more than 2000 plant species, including those belonging to the economically important families such as Solanaceae (tomato, potato, pepper, eggplant, tobacco), Fabaceae (soybean), Malvaceae (cotton) and Poaceae (rice, wheat, maize), for instance. Gall formation on the roots of infected plants dramatically impacts root growth, development, and nutrient uptake, resulting in subsequent stunted growth, leaf chlorosis, wilting, and significant yield losses. In addition, RKN infection can increase plant susceptibility to other pathogens, such as Fusarium crown and root rot for example.

Tomato (Lycopersicon esculentum Mill.) is an important, high-value crop with world-wide production of 180 million MT. In the United States, tomato is the second most important fruit vegetable in term of consumption, and in 2020 the United States produced around 1.4 million metric tons of tomatoes, worth more than $2.5 billion (Abrahamian et al., 2021). The top tomato producing states are California, Florida, Indiana, Ohio, Michigan, Tennessee, South Carolina, New Jersey, North Carolina, and Virginia. RKN, particularly M. incognita, can cause significant losses in tomato fruit yield. It has been estimated that under heavy RKN infection, the tomato yield loss could be as high as 50% particularly in regions with warm climate (Regmi and Desaeger, 2020).

Once introduced, RKNs are almost impossible to eradicate from the infested crops. Various management approaches to reduce RKN populations seem to be ineffective. Because the wide host ranges of RKN, including many vegetable and agronomic crops and weed species, crop rotation frequently fails to reduce population density. Chemical nematicides have proven effective. However, the use of methyl bromide was phased out in 2005, and the use of other nematicides is highly discouraged. Soil fumigation has been considered the main strategy for RKN management for decades. However, preplant soil fumigation is very costly and has been proven ineffective in many cases. For example, soil type, temperature, and organic matter content have been shown to dramatically reduce the effectiveness of fumigants (Hafez and Sundararaj, 2009). In addition, because several generations of nematode can occur in one season, preplant soil fumigation does not provide season-long protection. The development and use of RKN-resistant varieties are viewed as the foundation of a successful integrated nematode management strategy with fewer chemical inputs. In tomato, for example, commercially available nematode-resistant varieties are very limited and all rely on a single, dominant gene named Mi-1. Mi-1 is the only known source of resistance against RKN. This gene was first identified in the wild tomato Solanum peruvianum (Smith, 1944) and has been introduced into several tomato cultivars. However, resistance mediated by the Mi-1 gene has frequently become ineffective as a result of the temperature sensitivity of this gene in commercially available tomato cultivars. It has been shown that resistance is gradually reduced with increasing soil temperature, with a complete loss of resistance at 33° C. (Noling, 2016). Also, the frequent use of Mi-1 gene as the sole source of resistance led to genetic shift in RKN populations and development of Mi-1-virulent field populations capable of reproduction on resistant tomato cultivars and causing significant yield loss.

A key feature of RKN interactions with host plants is the release of nematode effector proteins into root cells and tissues. These effector proteins are synthesized in the nematode's esophageal gland cells, which consist of two subventral cells and one dorsal cell. These three large secretory cells differ in their activity throughout nematode parasitic stages. While the two subventral gland cells appear to be more active during the early stage of parasitism (i.e. root penetration, migration, and giant-cell formation), the single dorsal gland seems to be more active during the sedentary parasitic stages, when nematodes feed from the giant cells (Hussey 1989; Hussey and Mims 1990). Effector secretion from the esophageal gland cells to plant cells have been shown to be mediated through the stylet, a needle-like structure at nematode head (Hussey 1989; Hussey and Mims 1990). During the last two decades, great progress has been made in identifying nematode effectors from cyst, root-knot, and migratory nematodes (Bellafiore et al., 2008; Maier et al., 2013; Lilley et al., 2014; Gardner et al., 2018; Vieira et al., 2018). Functional studies of a limited number of nematode effectors provided insights into how parasitic nematodes reprogram host cells to their advantage to initiate feeding cells and establish successful interactive relationships with host plants (Hewezi and Baum 2013; Goverse and Smant, 2014; Hewezi, 2015; Fosu-Nyarkoand Jones, 2016; Siddique and Grundler, 2018; Hu and Hewezi, 2018; Gheysen and Mitchum, 2019; Mejias et al., 2019; Vieira and Gleason, 2019). Effector proteins often exert their parasitic functions via physical interactions with host proteins. Nematode effector proteins were found to interact with a wide range of host proteins with distinct functions, including transcription factors, protein kinases, stress- and defense-related proteins, cell wall-modifying enzymes, proteases, cytoskeletal proteins, RNA binding proteins, chromatin modifiers, and small signaling peptides, for instance (Hewezi and Baum 2013; Goverse and Smant 2014; Hewezi, 2015; Fosu-Nyarko and Jones, 2016; Siddique and Grundler, 2018; Hu and Hewezi, 2018; Gheysen and Mitchum, 2019; Mejias et al., 2019; Vieira and Gleason 2019). These interactions may alter enzymatic activity, signaling, cellular localization, protein association and stability of host interacting proteins.

Despite the functional characterization of a limited number of nematode effectors, the emerging picture is that nematode effectors target and manipulate numerous cellular processes and molecular functions to establish an infection. However, the complexity of effector-host protein associations is frequently masked due to the analysis of an individual effector and lack of large-scale interactome screens between nematode effectors and host proteins. Large-scale screens of effector-plant protein interactions have been recently performed in a limited number of studies (Mukhtar et al., 2011; Weßling et al., 2014; Petre et al., 2015; Petre et al., 2016, González-Fuente et al., 2020; Petre et al., 2021). These studies aimed to identify host proteins targeted by effectors from Pseudomonas syringae and Hyaloperonospora arabidopsidis (Mukhtar et al., 2011, Weßling et al., 2014), Melampsora larici (Petre et al., 2015), Puccinia striiformis f. sp. tritici (Petre et al., 2016), Ralstonia pseudosolanacearum and Xanthomonas campestris (González-Fuente et al., 2020), and Phytophthora infestans (Petre et al., 2021). These studies revealed that evolutionarily distant plant pathogens evolved independently to converge into a small number of host proteins. Also, it has been shown that host proteins targeted by two or more effectors of individual pathogen are of biological significance as mutants in genes encoding the host targeting proteins exhibit disease phenotypes of increased disease resistance or increased disease susceptibility (Mukhtar et al., 2011, Weßling et al., 2014). Thus, identifying host proteins interacting with nematode effectors will not only elucidate the molecular mechanisms underlying nematode parasitism of host plants but will point into host factors that can be targeted to enhance plant resistance to parasitic nematodes.

Therefore, discovery of new sources of genetic resistance to root-knot nematode is imperative to inhibit RKN infection in crops.

### BRIEF SUMMARY OF THE INVENTION

The instant invention pertains to the method of altering the synthesis of plant proteins that interact with effector proteins of Root knot nematodes (RKN) in establishing RKN infection. In certain embodiments, 11 different effectors from Meloidogyne incognita can be used as probes to identify plant proteins interacting with these effectors. In certain embodiments, the identified effector-interacting plant proteins can be modified to alter the expression of said the encoding genes. The mutations can completely abolish or significantly reduce (significantly inhibit), silence, or significantly increase the activity of these proteins.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1: Examples of positive yeast colonies growing on the selective quadruple dropout SD/-Ade/-His/-Leu/-Trp containing X-alpha-Gal. Blue color indicates the activity of alpha-galactosidase encoded by the MEL1 reporter gene, which is used for detecting GAL4-based yeast two-hybrid protein interactions.

FIG. 2: Number of tomato proteins interacting with each of the 11 tested Meloidogyne incognita effectors.

FIG. 3: Number of Meloidogyne incognita effectors interacting with each of the 20 tomato proteins targeted by at least two effectors.

FIG. 4: Meloidogyne incognita-tomato interactome network. Lines between effectors and tomato proteins indicate interactions.

FIG. 5: Classification of tomato interacting proteins into 12 functional Gene Ontology categories of biological processes.

FIG. 6: Virus-induced gene silencing of resistance gene candidates impacted tomato susceptibility to Meloidogyne incognita. Seven-day-old tomato seedlings were infiltrated with Agrobacterium strains containing each of the 7 constructed Tobacco rattle virus (TRV)-RNA1 (TRV2) TRV2 vectors mixed separately with the Agrobacterium strain containing the empty Tobacco rattle virus (TRV)-RNA1 (TRV1) TRV1 vector in a 1:1 ratio, and then inoculated with M. incognita second-stage juveniles 10 days later. The number of galls per plant root was determined 3 weeks after inoculation and used to determine susceptibly levels. Bars represent mean of at least 15 treated plants ±SE. Statistically significant differences between silenced plants and the control were determined using t-tests (*P<0.05). Solyc09g092380 and Solyc04g014570 significantly increased tomato susceptibility, whereas Solyc01g111520, Solyc08g069120, Solyc02g081210, Solyc05g056340, and Solyc01g100870 significantly enhanced plant resistance to M. incognita.

### BRIEF DESCRIPTION OF THE SEQUENCES

SEQ ID NOs: 1 to 118 provide amino acid sequences of tomato proteins that interact with RKN effector proteins that can confer RKN resistance when overexpressed, silenced, inactivated, or overexpressed and inactivated.

SEQ ID NOs: 119 to 129 provide amino acid sequences of Meloidogyne incognita effector proteins.

SEQ ID NOs: 130 to 247 provide DNA sequences of tomato proteins that encode amino acid sequences of SEQ ID NOs: 1 to 118 that interact with RKN effector proteins.

### DETAILED DISCLOSURE OF THE INVENTION

The present invention relates to novel and useful methods for introducing, in a reliable and predictable manner, RKN resistance into plants. The method involves identifying plant-effector interacting genes.

In this disclosure, the term “isolated nucleic acid” molecule means a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a variant protein used in the method of this invention has at least 80% sequence identity, preferably 85%, 90%, 91%, 92%, 93, 94%, 95%, 96%, 97%, 98%, 99%, or 100% identity, to a reference sequence), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence. The comparison window, in certain embodiments, refers to the full length sequence of a given polypeptide, for example a tomato-effector interacting protein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X ±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

An endogenous nucleic acid is a nucleic acid that is naturally present in a cell. For example, a nucleic acid present in the genomic DNA of a cell is an endogenous nucleic acid.

An exogenous nucleic acid is any nucleic acid that is not naturally present in a cell. For example, a nucleic acid vector introduced into a cell constitutes an exogenous nucleic acid. Other examples of an exogenous nucleic acid include the vectors comprising a heterologous promoter linked to an endogenous nucleic acid.

The subject invention provides for the use of “homologous nucleic acid sequences” or “homologs of nucleic acid sequences”. Homologs of nucleic acid sequences will be understood to mean any nucleotide sequence obtained by mutagenesis according to techniques well known to persons skilled in the art, and exhibiting modifications in relation to the parent sequences. For example, mutations in the regulatory and/or promoter sequences for the expression of a polypeptide that result in a modification of the level of expression of a polypeptide according to the invention provide for a “homolog of a nucleotide sequence”. Likewise, substitutions, deletions, or additions of nucleic acid to the polynucleotides of the invention provide for “homologs” of nucleotide sequences. In various embodiments, “homologs” of nucleic acid sequences have substantially the same biological activity as the corresponding reference gene, i.e., a gene homologous to a native gene would encode for a protein having the same biological activity as the corresponding protein encoded by the naturally occurring gene. Typically, a homolog of a gene shares a sequence identity with the gene of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

In certain embodiments, genetic modifications are made to plants or plant cells that “significantly reduces or abolishes the expression of a gene.” This phrase refers to a reduction of gene expression in an amount of at least (or at least about) 30% as compared to a non-genetically modified plant from which the genetically modified plant was derived (e.g., a tomato plant). Thus, plants that are genetically modified to exhibit significantly reduced or abolished expression of a gene exhibit a reduction in gene expression or expression of an active protein that can range from about 30% to about 99.99% about 40% to about 99.99%, about 50% to about 99.99%, about 60% to about 99.99%, about 70% to about 99.99%, about 80% to about 99.99%, about 90% to about 99.99% or are devoid of expression (expression is abolished) of the gene or an active protein encoded by the gene.

In certain embodiments, genetic modifications are made to plants or plant cells that “significantly increases the expression of a gene.” This phrase refers to an increase of gene expression in an amount of at least (or at least about) 30% as compared to a non-genetically modified plant from which the genetically modified plant was derived (e.g., a tomato plant). Thus, plants that are genetically modified to exhibit significantly increased expression of a gene exhibit an increase in gene expression or expression of an active protein that can range from 30% to about 99.99% about 40% to about 99.99%, about 50% to about 99.99%, about 60% to about 99.99%, about 70% to about 99.99%, about 80% to about 99.99%, or about 90% to about 99.99%.

As used herein, “co-expressed” or “co-expression” is when the expression patterns of two or more genes are correlated across multiple tissues and/or with the same RKN effector protein.

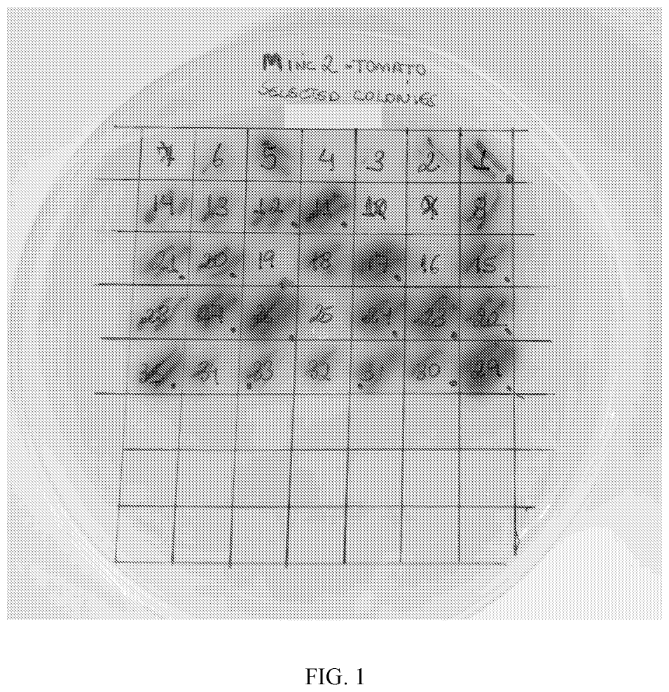

To determine the interactome of RKN effector proteins with plant proteins, yeast two-hybrid (Y2H) screens can be performed for each RKN effector separately. In certain embodiments, the RKN effectors are SEQ ID NOs: 119-129. The Y2H screen pipeline can include primary screens using the stringent quadruple dropout SD/-Ade/-His/-Leu/-Trp medium, secondary phenotyping screens using the quadruple dropout SD/-Ade/-His/-Leu/-Trp/X-alpha-Gal medium to visualize the activity of alpha-galactosidase encoded by the MEL1 reporter gene (positive yeast colonies expressing the Mell reporter gene turn blue in the presence of the chromogenic substrate X-alpha-Gal) (FIG. 1), and finally growth rate in the SD/-Ade/-His/-Leu/-Trp liquid medium. Additional confirmation including yeast co-transformation assays of bait and prey plasmids in the Y2HGold yeast strain as previously described (Hewezi et al., 2008; Hewezi et al., 2010; Hewezi et al., 2015; Hewezi et al., 2016, each of which are hereby incorporated by reference in its entirety).

The disclosure provides that the amino acid sequences listed in Table 1 (SEQ ID NOS: 1 to 118) provide resistance or susceptibility to a pathogen in a plant cell or a plant, particularly a tomato plant cell or a tomato plant. For example, overexpressing in a plant cell or a plant, particularly, a tomato plant cell or a tomato plant, one or more genes that encode an amino acid sequence selected from SEQ ID NOs: 1 to 118 or homologs thereof renders the plant cell or the plant resistant to RKN. Also, inactivating in a plant cell or a plant, particularly, a tomato plant cell or a tomato plant, one or more genes that encode an amino acid sequence selected from SEQ ID NOs: 1 to 118 or homologs thereof renders the plant cell or the plant, particularly resistant to RKN. Also, silencing in a plant cell or a plant, particularly, a tomato plant cell or a tomato plant, one or more genes that encode an amino acid sequence selected from SEQ ID NOs: 1 to 118 or homologs thereof renders the plant cell or the plant, particularly resistant to RKN. Also, decreasing the expression in a plant cell or a plant, particularly, a tomato plant cell or a tomato plant, of one or more genes that encode an amino acid sequence selected from SEQ ID NOs: 1 to 118 or homologs thereof renders the plant cell or the plant, particularly resistant to RKN.

TABLE 1
SEQ ID NOs: 1 to 118 providing amino acid
sequences that confer RKN resistance when
overexpressed, inactivated, silenced, or
overexpressed and inactivated.

| | Protein Name | Protein Sequence |
|---|---|---|
| SEQ | Solyc01g008960.3 | MAEEETNGAAAEGLPPPPPVPPDFT |
| ID | | PAKTEIEPVKKKILRVPMARRGVGN |
| NO: | | KGQKIQILTNHFKVNVNNVDGHFFH |
| 1 | | YSVALFYEDGRPVDGKGVGRKVLDT |
| | | VHETYDTELAGKDFAYDGEKSLFTI |
| | | GALPRNKMEFTVVLDDVISNRNNGN |
| | | SSPGGHGSPNEADRKRLRRPYQSKT |
| | | FKVEISFAAKIPMQAIANALRGQES |
| | | ENSQEALRVLDIILRQHAAKQGCLL |
| | | VRQSFFHNDPKNFVDVGAGVLGCRG |
| | | FHSSFRTTQSGLSLNIDVSTTMIIQ |
| | | PGPVVDFLIANQNAKDPFSLDWAKA |
| | | KRVLKNLRVKTTPTNQEYKITGLSD |
| | | RPCREQLFTLKQKGKDADGEVQTTE |
| | | VTVFDYFVNHRNIELRYSADLPCIN |
| | | VGKPKRPTFFPIELCSLVSLQRYTK |
| | | SLSTFQRSSLVEKSRQKPQERMQVL |
| | | SNALKINQYDAEPLLRSCGISISNN |
| | | FTQIEGRVLPPPKLKTGGDDFVPRN |
| | | GRWNFNNKRLVDPTKIERWAVVNFS |
| | | ARCNVQGLVSDLIKCGKQKGIMVED |
| | | PFDVFEESPQVRRAPPLVRVEKMFE |
| | | QVQSKLPGAPKFLLCLLPERKNCDV |
| | | YGPWKRKNLAEYGIVTQCIAPTRVN |
| | | DQYITNVLLKINAKLGGLNSMLTVE |
| | | HSPAIPMVSKVPTIILGMDVSHGSP |
| | | GQSDVPSIAAVVSSRQWPSISRYRA |
| | | SVRTQSPKVEMIDNLFKRTSDTEDD |
| | | GIMREALLDFYVSSGKRKPEHIIIF |
| | | RDGVSESQFSQVLNVELDQIIEACK |
| | | FLDEKWSPKFVVIVAQKNHHTKFFQ |
| | | PNDPNNVPPGTIIDNKVCHPRNYDF |
| | | YLCAHAGMIGTTRPTHYHVLYDELG |
| | | FSADDLQELVHNLSYVYQRSTTAIS |
| | | VVAPICYAHLAATQMGQWMKFEDAS |
| | | ETSSSHNGVTNAGPVSVPQLPKLEE |
| | | KVSSSMFFC* |
| SEQ | Solyc01g014840.3 | MSSICPQNWKNDVFLSFRGKDTRKT |
| ID | | FVGHLNYALKQKGIHTFKDDERLER |
| NO: | | GKTISPELVKAIEESRFAIVVFSKN |
| 2 | | YASSTWCLDELVKIMECKKELGQTV |
| | | IPIFYDVDPSHVTKQSETFAKSFAI |
| | | HEENLKDDVEKVLCWRDAFRQAGKI |
| | | AGYDLPNGYDGYESNCIQHVVEGIL |
| | | SKLCQVISTIENDLVGMESRVQEVS |
| | | SLLRMETLDVRFIGIWGMGGIGKTT |
| | | IARAVFGKYWGQFEGVCFLDNVVEM |
| | | QKTHGLQYLQGVLLSKILKVSITIA |
| | | SVYEGMRIIKERLRSMKVLIILDDV |
| | | IQKDQLEMLVGGRDWFGIGSRILIT |
| | | TTDKHLLHNHVVDEVYSVNLMTRDE |
| | | AIELFSLHAFKQRIPDKDFEELLNQ |
| | | VVQTAGFLPLALKILGSSLCGLDKI |
| | | VWKCTVDRLKDMPDDVLAKLKISFD |
| | | GLSPSDQRLFLDIGCFYRGKLRSSV |
| | | GEMLESCDIGSTIRVLIEKSLLFVS |
| | | PYDTIEMHDLIQEMAWHIVRQGDTR |
| | | RSRIWLPEDIEDLFTGNLEAEAVEG |
| | | LSIPRDYVRKRDISCYNINETFRKM |
| | | KRLRVLEVRATNFCSIDPITHLPSS |
| | | IRWLDWEGCPLNSLPQSFEPSKLLR |
| | | LDILESTTLQKLWSISKGLDKLKAL |
| | | YLSYCEKLEEVPSFDIMPNLERVKL |
| | | EGCNSLREVSPSFGVLMKLTSMELI |
| | | DCPSLKKIPSCVEMGSLKSLKLSCL |
| | | PKLRELPETKGMHRLLTLELTNCQY |
| | | LEKLPNFDQMESLETLKLSCLPKVT |
| | | IFPPPDGMHCLQDIVIEYVPIVELP |
| | | ASIGNLRSLKQLRLSHCKDLVSIPN |
| | | SFCCLKNLRALLIYNCKRLIYLPEK |
| | | IGDLKLLKKLVVSGTSISQIPTSVT |
| | | DLRELSFLSFSRWSGYRQGATFLFP |
| | | PSSGVSLPLRVLKLKKNRLCSAEHF |
| | | PDVGGLSSLAHLDLTRNDESTNQLF |
| | | HYLDITFCEKLALPELPPFIKELYA |
| | | CDPLILKSIPDFLTKYSDLYSVSFT |
| | | RHIENKGELTDLLLFVLRMISVASQ |
| | | CEKR |
| | | PPLSLFFPGDIKWKWFTYYRKEQTK |
| | | RFSTQLDPRWYESKFIKGFVLCFRV |
| | | PSVIGQNQKCSNDKSQRGSSRRFGC |
| | | TKVTAKLVQRYNRQEHDVLQKKCLI |
| | | VARQTFCSHGSKYSICFNYIPFVAC |
| | | EKGKKLLNEYCFFEASLEPDIATKW |
| | | GLLLVYEKKIQQIDQSTIAVQCDVD |
| | | SPNSDLFRECNDDQGHRKEDVSVKR |
| | | RRLDLRQVMKDSCSTSIVPDQQMET |
| | | PSSSAAQSFQHRDESCSSGQPQTTQ |
| | | IPQADDPRVDEAIDEATSCVVVDQP |
| | | DVFDVSPDEHKDNSMTTGSGCSVVS |
| | | QQLETQCTSGKSQTLRLFPDHS* |
| SEQ | Solyc01g073710.2 | MGVVIIDGSTIRDFVNDEAAFTKSI |
| ID | | DKGFTDLDLNNDGVLSRSELRKAFE |
| NO: | | SLHLIESHFGVDVAATPEELTDLYD |
| 3 | | SIFEKFDCDHNGTVDREEFGNEMRK |
| | | IMQAIADGLGSSPIQMALDDSEQSL |
| | | IKQAADLEASKLPA* |
| SEQ | Solyc01g087240.3 | MPTIAKHDGRQLETGLRITSLSTTQ |
| ID | | MQPWWHGYGDNTMPLASENAVAQEK |
| NO: | | SEGGNQDKETKALAMESGSDGNNEQ |
| 4 | | YKQHLKHFAPTTAAIMAEQQKELTG |
| | | HSAMLASYPYPDMQYGGMMTYGAPV |
| | | HPHLFEIHHARMPLPLDMEEEPVYV |
| | | NAKQYHGILRRRQIRAKAELERKAI |
| | | KARKPYLHESRHQHAMRRARGTGGR |
| | | FLNTKKLNDMDCTPTETQKYGATIA |
| | | THSGNSSGSGSSDQGGKEGSTVQDM |
| | | HKGHSQSFTTGNGHGSSVYFSASSG |
| | | SEQGNGHYGHGSWSLLVNQASQGAA |
| | | SSN* |
| SEQ | Solyc01g087900.3 | MVCIRKATIDDLLAMQACNLLCLPE |
| ID | | NYQMKYYFYHILSWPQLLYVAEDYN |
| NO: | | GKIVGYVLAKMEEETTECHGHITSL |
| 5 | | AVVRTHRKLGLATKLMTAAQNAMEQ |
| | | VFGAEYVSLHVRKSNRAAFKLYTET |
| | | LGYKIHDMEAKYYADGEDAYDMRKE |
| | | LKGKKHHQHHHHHHHHGGGCCSGEV |
| | | KVGPKEGAAETKAA* |
| SEQ | Solyc01g090710.3 | MYCGSILTVSNFNYL*ILFLIIKLM |
| ID | | EYWGSIEKPIVLLGCAYLSWKIVTH |
| NO: | | IWSFMNIEKDPITVLVTGAAGQIGY |
| 6 | | ALVPMIARGAMLGLDQPVIIHMLDI |
| | | EPAAESLKGVKMELIDAAFPLLKDV |
| | | VATTNVVEACKGVNVAVMVGGFPRK |
| | | EGMERKDVMTKNVSIYKAQASALEQ |
| | | HAAPDCKVLVVANPANTNALILKEF |
| | | APSFPAKNITCLTRLDHNRALGQIS |
| | | EKLNVHVGDVKNVAIWGNHSSTQYP |
| | | DVNHATVKTAAGEKPVRELVANDQW |
| | | LNTEFITTVQQRGAAIIKARKLSSA |
| | | LSAASSACDHIHDWVLGTPKGTWVS |
| | | MGVYSDGSYGIPAGLIYSFPVTCEK |
| | | GEWSIVQGLKIDEFSRAKMDATAKE |
| | | LAEEKSLAYSCLN* |
| SEQ | Solyc01g091620.3 | MNVMRRLKSIASGRSSVSDPGGDSS |
| ID | | IKRVKVEKEVDQRVVGETQMEEGCT |
| NO: | | TTTVPKEDMASTSMETTAGSTSTMD |
| 7 | | IRLENSELDELPKEMHEMKIKDEKD |
| | | DKADSLDDNLKDMEPAVVSGNGTET |
| | | GQIIVTTVSGRNGQEKQTLSYMAER |
| | | VVGTGSFGVVFQAKCLETGESVAIK |
| | | KVLQDRRYKNRELQIMRTLDHPNVV |
| | | KLRHCFYSTTEKNEVYLNLVLEYVS |
| | | ETVYRVSRHYSRMNQHMPNIYVQLY |
| | | TYQICRALNYMHGVLGVCHRDIKPQ |
| | | NLLVNPHSHQLKLCDFGSAKMLVPG |
| | | EPNISYICSRYYRAPELIFGATEYT |
| | | TAIDMWSAGCVMAELLLGQPLFPGE |
| | | SGVDQLVEIIKILGTPTREEIRCMN |
| | | PNYTEFKFPQIKAHPWHKIFHKRMP |
| | | PEAVDLVSRLLQYSPTLRCTALEAC |
| | | AHPFFDSLREPNACLPNGRPLPPLF |
| | | NFSPQELSGVPAELRKRLIPEHLRK |
| | | * |
| SEQ | Solyc01g095410.3 | MPKNKGKGGKNRKRGKNEADDEKRE |
| ID | | LVFKEDGQEYAQVLRMLGNGRCEAM |
| NO: | | CIDGTKRLCHIRGKMHKKVWIAAGD |
| 8 | | IILVGLRDYQDDKADVILKYMPDEA |
| | | RLLKAYGELPENTRLNEGIANLDEE |
| | | DENAADDYIEFEDEDIDKI* |
| SEQ | Solyc01g095700.3 | MESQFIERYHSHQPSEHQCSSSLVK |
| ID | | HIKAPVDIVWSLVRRFDQPQKYKPF |
| NO: | | ISRCTVKGDLTIGSVREVNVKSGLP |
| 9 | | ATTSTERLELLDDEEHILGIRIVGG |
| | | DHRLKNYSSVITVHPETLDGRPGTL |
| | | VIESFVVDVPEGNTKEETCYFVKAL |
| | | INCNLKSLADVSERMAMQGGVLPVS |
| | | VNWSSSNQIEI* |
| SEQ | Solyc01g096580.3 | MAAYAAMKPTKPGLEEPAEMVHKIR |
| ID | | ITLSSKNVKNLEKVCADLVRGAKDK |
| NO: | | RLRVKGPVRMPTKVLNITTRKSPCG |
| 10 | | EGTNTWDRFELRVHKRVIDLFSSPD |
| | | VVKQITSITIEPGVEVEVTIAES* |
| SEQ | Solyc01g096760.3 | MDGGSSNAPVQQLTAEAAAQLQEGI |
| ID | | GLVLTRWTALQMAIENEWGGRDTRE |
| NO: | | KSNQLNVDIFSAFTQSKEKVYMDDI |
| 11 | | EEILDEFMISLNTEVNDGSLEEVAE |
| | | KLMYMHEECLEGDENSIKVLRETNV |
| | | GRRPATYVRQDASDDDDSSNDGDDR |
| | | NENFGNNSSDMAIDSMESQPSLGQD |
| | | MIVEPVRKQPAEVDPDGWTTVSTKR |
| | | NKGRRN* |
| SEQ | Solyc01g097520.4 | MAEANSYAALTKAFSGLGVDENLFI |
| ID | | STLGKWNRHQRESYRVSTPGFFRED |
| NO: | | KRQFQRWDDQHILQLRQEFLRLKDA |
| 12 | | VVLYTMHPWERDARLFKEALLLQVP |
| | | KHDVLIEIACTRSSEDLLGARKAYH |
| | | SLFEHSIEEDIAFHVRSSERKLLVA |
| | | LVSSYRYEGPRVNDDLAKSEAKIFV |
| | | NAIKNANKKKLIDEDEEIVRILSTR |
| | | SKLHLKAIYSHYKEITGNFLDEDLE |
| | | CDLTLKQVVQCLCIPKAYFSKILIA |
| | | SLRLDVDESAKDSVTRVIVSRADDD |
| | | DMQQIKEEFQSKYGTTLAEKIAEVA |
| | | NGSYKDFLLTIIAKSE |
| SEQ | Solyc01g100870.3 | MGLSFGKLFSRLFAKKEMRILMVGL |
| ID | | DAAGKTTILYKLKLGEIVTTIPTIG |
| NO: | | FNVETVEYKNISFTVWDVGGQDKIR |
| 13 | | PLWRHYFQNTQGLIFVVDSNDRDRV |
| | | VEARDELHRMLNEDELRDAVLLVFA |
| | | NKQDLPNAMNAAEITDKLGLHSLRQ |
| | | RHWTGTFRAHVLPLEKGCMRGLTGS |
| | | QTTLQTRVEHEIHDFCSYIFVNIDF |
| | | KQCLEHLCTC* |
| SEQ | Solyc01g101060.4 | METFLFTSESVNEGHPDKLCDQISD |
| ID | | AVLDACLEQDPESKVACETCTKTNL |
| NO: | | VMVFGEITTKAIVDYEKIVRDTCRN |
| 14 | | IGFVSDDVGLDADNCKVLVYIEQQS |
| | | PDIAQGVHGHLTKRPEEIGAGDQGH |
| | | MFGYATDETPELMPLSHVLATKLGA |
| | | RLTEVRKNGTCAWLRPDGKTQVTVE |
| | | YSNDNGAMVPIRVHTVLISTQHDET |
| | | VTNDEIARDLKEHVIKPVIPEKYLD |
| | | ENTIFHLNPSGRFVIGGPHGDAGLT |
| | | GRKIIIDTYGGWGAHGGGAFSGKDP |
| | | TKVDRSGAYIVRQAAKSIVASGLAR |
| | | RCIVQVSYAIGVPEPLSVFVDTYGT |
| | | GKIPDREILKIVKENFDFRPGMMSI |
| | | NLDLKRGGNRRFLKTAAYGHFGRDD |
| | | PDFTWEVVKPLKWEKPQD* |
| SEQ | Solyc01g103490.3 | MALKLSAAEAAKVAIQSIGCGYDIS |
| ID | | LDLRLKYCKGHPDNSRLIEIDENES |
| NO: | | GEVVLPGGISIPNVSKSIKCDKGEH |
| 15 | | TRFGSDVLSFQQMSEQFNQEVSVNG |
| | | KIPSGLFNTMFEFSGSWQKDAAYTK |
| | | TLAFDGVFITLYSVALEKSQMVLCD |
| | | YVKKEVPSTWDPAALARFIEKFGTH |
| | | VIVGIKMGGKDVIYMKQQHSSSLLP |
| | | ADVQKRLKTMADKRFSGANEQGGVE |
| | | SQQTHQNEKFEIREHRLRFADPNIS |
| | | GSYAHKEDTVCICKRRGGSDSRGLM |
| | | HNQWLQSVDQEPDVISMSFIPITSL |
| | | LNGISGSGFLSHAINLYLRYKPPIE |
| | | ELHQFLEFQLPRQWAPVFGDLPLGP |
| | | QRRHLSFPSLQFSLLGPRLYVNTTP |
| | | VDVGKRPVTGLRLYLEGRRSNRLAV |
| | | HLQHLSSLPKVFQLEDDPKGNFRRE |
| | | SYDRKYYEKVQWKNFSHVCTAPVES |
| | | DEDLNIVTGAQLELGDYGFKKVLFL |
| | | RLRFSTVMGATMVKHAEWDGSPGLA |
| | | RKSGLISTLISQHFTSVQKPPPRPD |
| | | DVNINSAIYPGGPPVPVQAPKLLKF |
| | | VDTTEMTRGPQEPPGYWVVSGARLM |
| | | VEKGRISVRVKYSLLTVIQPDDEIS |
| | | E* |
| SEQ | Solyc01g105070.3 | MAYYNYNLINKMVTIIFILVLVIVD |
| ID | | VTMVFGQGTRVGFYSSTCPRAESIV |
| NO: | | QSTVRSHFQSDPTVAPGLLRMHFHD |
| 16 | | CFVQGCDGSILISGTGTERTAPPNS |
| | | NLRGFEVIDDAKQQIEAVCPGVVSC |
| | | ADILALAARDSVLVTKGLTWSVPTG |
| | | RRDGRVSSASDTSNLPGFTESVDAQ |
| | | KQKFAAKGLNTQDLVTLVGGHTIGT |
| | | SACQFFSYRLYNFNSTGGPDPSIDA |
| | | TFLSQLQALCPQNGDGSKRVALDTG |
| | | SVNNFDTSYFSNLRNGRGILESDQI |
| | | LWTDASTKVFVQRYLGLRGFLGLRF |
| | | GLEFGKSMVKMSNIEVLTGTNGEIR |
| | | KVCSAFN* |
| SEQ | Solyc01g105340.4 | MLANQKSKAVTYRKLARSYHPDVNK |
| ID | | EPGAEQKFKEISNAYEVLSDDEKRS |
| NO: | | IYDKYGEAGLKGAGAGMGDFSNAFD |
| 17 | | LFESLFDGFGGMGGMGGMGGRGSRS |
| | | RATEGEDQGYNLVLNFKEAVFGVEK |
| | | EIEISRLETCGTCDGSGAKPGTKPS |
| | | TCNTCGGQGQVVSSARTPLGVFQQV |
| | | TTCSSCGGTGEISTPCGTCNGDGRV |
| | | RKSKRISLKVPPGVDSGSRLRVRSE |
| | | GNAGRRGGPPGDLFVMIEVLPDPVL |
| | | KRDDTNILYNCKVSYIDAILGTTMK |
| | | VPTVDGMVDLKIPAGTQPGTTLVMA |
| | | KKGVPFLSKPNMRGDQLVRVQVEIP |
| | | KRLSSEERKLIEELANLNKPKAATN |
| | | SKR* |
| SEQ | Solyc01g109660.2 | MADVEYRCFVGGLAWATTDQTLSEA |
| ID | | FSQYGEVVESKIINDRETGRSRGFG |
| NO: | | FVTFKDEQAMRDAIEGMNGQDLDGR |
| 18 | | NITVNEAQSRGGGGGGGGRGGGGYG |
| | | GGRREGGGGGYGGGGGYGGGRREGG |
| | | GGGYGGGGYGGGRREGGYGGGGGGY |
| | | GGGDRYNDRSSRGGGGGGSDGNWRN |
| | | * |
| SEQ | Solyc01g111330.4 | MDFSYSSPSHFILSLCFALLILSRG |
| ID | | VLGATFTFUNKCEYTVWPGILANAG |
| NO: | | SPTLQSTGFELPQESSRVFTAPTGW |
| 19 | | SGRFWGRTSCTFEGSGSGSCGTGDC |
| | | GSGEVECNGAGAAPPATLAEFTLGT |
| | | VGQDFYDVSLVDGYNLPMVVEASGG |
| | | SGMCATTGCVTDLNQICPSELRSGG |
| | | GEACRSACEAFNKPEYCCSGAYNTP |
| | | STCKPSMYSQMFKSACPRSYSYAYD |
| | | DPTSTFTCSGADYTITFCPSTPSQK |
| | | STKDTTTPNGDGSYGATGTGVVDPA |
| | | TGGIESSSGQMGTGSGTGFEFGAGT |
| | | GYGSGTETQPGSGSQAMLADGSYLA |
| | | GLAMGESSRAFFPLNLAGSALATTL |
| | | VSLVFL* |
| SEQ | Solyc01g111450.3 | MARYDRAITVFSPDGHLFQVEYAME |
| ID | | AVRKGNAAVGVRGTDTVVLGVEKKS |
| NO: | | TPKLQDSRSVRKIVNLDDHIALACA |
| 20 | | GLKADARVLVNKARIECQSHRLTVE |
| | | DPVTVEYITRYIAGLQQKYTQSGGV |
| | | RPFGLSTLIIGFDPHTGVPSLYQTD |
| | | PSGTFSAWKANATGRNSNSTREFLE |
| | | KNYKETSGQETVKLAIRALLEVVES |
| | | GGKNIEVAVMTKEHGLKQLEEAEID |
| | | AIVAEIEAEKAAAEAAKKAPPKET* |
| SEQ | Solyc01g111520.3 | MGFVSTILGFCGFGVGVSCGLTIGY |
| ID | | YLFIYFQPCDVKDPVIRPLVERDSK |
| NO: | | SLQQLLSEIPLWVKCPDYDRVDWLN |
| 21 | | KFIEYMWPYLDKAICRTAKDIAAPI |
| | | IAEQIPKYKIDSVEFETLTLGSLPP |
| | | TFQGMKVYVTEEKELIMEPSIKWAG |
| | | NPNVTVAVKAFGLKATVQVVDLQVF |
| | | AAPRITLKPLVPSFPCFANIFVSLM |
| | | EKPHVDFGLKLLGADLMSIPGLYRF |
| | | VQETIKDQVANMYLWPKSLEVQILD |
| | | PSKAMKKPVGVLHVKILRAMNLKKK |
| | | DLLGASDPYVKLKLTESKLPSKKTP |
| | | VRHKNLNPEWNEEFNMVVKDPESQA |
| | | LELSVYDWEKIGKHDKMGMNVIPLK |
| | | DLTPDETKTMTLSLLKNMDANDSQN |
| | | DKDRGQIMVELTYKPFKEDELPKDF |
| | | EDNDAAHKVPEGTPPGGGVLMIIVH |
| | | EAQDVEGKHHTNPYVKILFKGEERK |
| | | TKQVKKNRDPRWEEEFTFVLEEPPV |
| | | NDRVHMEVVSTSTRIGLLHPKETLG |
| | | YVDINLSDVVSNKRINEKYHLIDSK |
| | | NGRLQVELQWRTAS* |
| SEQ | Solyc01g112000.4 | MSLFFLVLLLFVSSVTACDRCVHRS |
| ID | | KVAYFSSSSALLSGACGYGNLAIGF |
| NO: | | NGGRLAAAVPKLYDNGAGCGACYQI |
| 22 | | RCTDPKICSKHGTTVMVTDLNTNNQ |
| | | TDFVISSRAFMAMAIEGKAQDVHKL |
| | | GIADVEYKRVPCDYKGKNLAVRVDE |
| | | SSQKPHYLAISIFYQGGQTEIVSVD |
| | | VAQVGSSNWNFLSRNHGAIWDTSRV |
| | | PSGALQFRFVVTAGYDGKWIWAKSV |
| | | LPSDWKNGVVYDTGLQITDIAQEGC |
| | | SPCDDGSWKLN* |
| SEQ | Solyc02g021640.3 | MACMKLGSKTDAFQRKGQAWFCTTG |
| ID | | LPSDIVVEVGEMTFHLHKFPLLSRS |
| NO: | | GVMEKRIAEASEGEDGCVIELNDIP |
| 23 | | GGAKTFELVAKFCYGVKLELTAGNA |
| | | VYLRCAAEHLDMTEEYGEGNLISQT |
| | | EIFLNQVVLRSWKDSLKALQTCDDV |
| | | LPYAEELRITKRCIDSLAVKACTDP |
| | | NLFGWPVMEHVGPLQSPGGSILWNG |
| | | ISTGARPKHSSSDWWYEDASTLSLP |
| | | LYKRLISAMESQGVKQDIVAGSLSY |
| | | YAKKYLPGLNRRQTSSESTNRLAPV |
| | | GLGSSLSEEDQKLLLEEVDNLLPMQ |
| | | KGLVPTKFLFGLLKTALILRACPSC |
| | | ISNLEKRIGMQLDQATLEDLLMPNF |
| | | SYSMETLYNVDCVQRILEHFLAMDQ |
| | | GTGGESPCSIDDEQLIGSPSLTPIT |
| | | MVAKLIDGYLAEVAPDVNLKLPKFQ |
| | | TLAASVPEYARPLDDGLYRAIDIYL |
| | | KSHPWLGESDREQLCRLMDCQKLSL |
| | | EACTHAAQNERLPLRIIVQVLFFEQ |
| | | LQLRTSIAGCFLVSENLDEGSRQLR |
| | | SGTVGPNEGGWATAVRENQVLKVGM |
| | | DSMRVRVSELEKECSTMRQEIEKLG |
| | | RSKGSSTWGNVSKKFGFKMKSQMCS |
| | | AQEGSVSNQNKINSKAIKDDKAKEN |
| | | YRKH* |
| SEQ | Solyc02g067030.4 | FFYGPLWPKKVHYWLAVADSVEGKH |
| ID | | QRKMDGTAVQGTSSVDSFLRNYKLG |
| NO: | | KTLGIGSFGKVKIAEHTLTGHKVAV |
| 24 | | KILNRRKIRNMDMEEKVRREIKILR |
| | | LFMHPHIIRLYEVIETPSDIYVVME |
| | | YVKSGELFDYIVEKGRLQEDEARNF |
| | | FQQIISGVEYCHRNMVVHRDLKPEN |
| | | LLLDSKWNVKIADFGLSNIMRDGHF |
| | | LKTSCGSPNYAAPEVISGKLYAGPE |
| | | VDVWSCGVILYALLCGTLPFDDENI |
| | | PNLFKKIKGGIYTLPSHLSAGARDL |
| | | IPRMLIVDPMKRMTIPEIRLHPWFQ |
| | | AHLPRYLAVPPPDTTQQAKKIDEEI |
| | | LQEVVKMGFDRNNLTESLRNRVQNE |
| | | GTVAYYLLLDNRHRVSTGYLGAEFQ |
| | | ESMEYGYNRINSNETAASPVGQRFP |
| | | GIMDYQQAGARQFPIERKWALGLQS |
| | | RAHPREIMTEVLKALQELNVCWKKI |
| | | GQYNMKCRWVPSLPGHHEGMGVNSM |
| | | HGNQFFGDDSSIIENDGATKLTNVV |
| | | KFEVQLYKTREEKYLLDLQRLQGPQ |
| | | FLFLDLCAAFLAQLRVL* |
| SEQ | Solyc02g067860.4 | MSSNIIHDADGSASRIVLKEHTGST |
| ID | | AEVLFQGGQVVSWKNERREELLFRS |
| NO: | | NKTLWKPPKAFRGGIPISFPQFGTF |
| 25 | | GSLERHGFARNRVWELDDSPSPLPA |
| | | ANNQSTVDLILKSTEEDLKTWPHRF |
| | | EVRLRISLSAGKLTLIPRVRNIDNK |
| | | PFSFTFSLRNYLSVSDISEVRVEGL |
| | | ETLDYFDNLQRGERYTEQADAITFD |
| | | SETDRVYLSTPTKTAIIDHEKKRTY |
| | | VLQKEGMADAVVWNPWDKKAKSLPD |
| | | LGDQDYKKMLCVDSAAVETPIILKP |
| | | SEEWRGRQELSTVSSSYCSGQLDPR |
| | | KVIYG* |
| SEQ | Solyc02g068130.4 | MEMQSEFDRLLFFEHARKSAETAYA |
| ID | | QNPLDADNLTRWGGALLELSQFQPV |
| NO: | | AESKQMISDATSKLEEALTVNPEKH |
| 26 | | DALWCLGNAHTSHAFLTPDMDEAKV |
| | | YFEKATQCFQQAFDADPSNDLYRKS |
| | | LEVTAKAPELHMEIHRHGPMQQPMA |
| | | AEPSTSTSTKSSKKKKSSDLKYDIF |
| | | GWVILAVGIVAWVGFAKSNMPPPPP |
| | | PHPQ* |
| SEQ | Solyc02g069090.3 | MKHIATFLLLVSVSTLVLQVVAENP |
| ID | | ISQAKAESAILQDSIVKQVNENEKA |
| NO: | | GWRAALNPQFSNFTVSQFKRLLGVK |
| 27 | | PTRKGDLKGIPILTHPKLLKLPQEF |
| | | DARVAWPQCSTIGRILDQGHCGSCW |
| | | AFGAAESLSDRFCIHYGLNISLSAN |
| | | DIIACCGYLCGDGCDGGYPLEAWKY |
| | | FVRKGVVTEECDPYFDNKGCSHPGC |
| | | EPGYPTPQCKRKCVKENLLWSKSKH |
| | | FGINAYLINSDPYSIMTEVYKNGPV |
| | | EVSFTVYEDFAHYKSGVYKHINGEE |
| | | MGGHAVKLIGWGTSEDGEDYWLLAN |
| | | QWNRGWGDDGYFKIRRGTNECGIEE |
| | | EVVAGMPSAKNLNVELDVSDALLDA |
| | | SM* |
| SEQ | Solyc02g069100.4 | MALTLKSLITPLLFGAFFILILQVA |
| ID | | AEKPITEAKLESAILQDSIVKQVNE |
| NO: | | NAEAGWKAAFNPQLSNFTVSQFKRL |
| 28 | | LGVKPAREGDLEGIPVLTHPKLKEL |
| | | PKEFDARKAWPQCSTIGRILDQGHC |
| | | GSCWAFGAVESLSDRFCIHYNLSIS |
| | | LSVNDLLACCGFLCGSGCDGGYPIA |
| | | AWRYFKRRGVVTEECDPYFDTTGCS |
| | | HPGCEPLYPTPKCHRKCVKGNVLWR |
| | | KSKHYGVNAYRVSHDPQSIMAEVYK |
| | | NGPVEVSFTVYEDFAHYKSGVYKHV |
| | | TGGNMGGHAVKLIGWGTSEQGEDYW |
| | | LIANSWNRGWGEDGYFKIRRGTNEC |
| | | GIEHSVVAGLPSARNLNVELGDAVL |
| | | DASM* |
| SEQ | Solyc02g080630.4 | MGFSVFLFTFLLQHLLIFNHVFYLC |
| ID | | STDRCSNRFFTLPRLISLSLNLNMA |
| NO: | | EASAPAVPSTELLEWPKKDKRRMLH |
| 29 | | AVYRVGDLERTIKFYTECFGMKLLR |
| | | QRDIPEEKYSNAFLGFGPEESHFVV |
| | | ELTYNYGVDKYDIGTGFGHFAIATP |
| | | DVYKLVEEIKAKGGTVTREPGPVKG |
| | | GSSVIAFVKDPDGYLFEIIQRESTP |
| | | EPLCQVMLRVGDLERAIKFYEKALG |
| | | MQVVKKVDRPEHKYTIAMMGYAPEK |
| | | ETTVIELTYNYGVTEYTKGNAYAQI |
| | | AIGTDDVYKSAKVVNLAIQELGGKI |
| | | TRQPGSIPGLNTKITSFLDPDGWKT |
| | | VLVDNKDFLKELESSK* |
| SEQ | Solyc02g081210.3 | MLLLSLLVAFALFSPTPFVHSHYNW |
| ID | | SPSSSSTSNFQSEWRPARATYYAAA |
| NO: | | DPRDVVGGACGYGDLERNGYGKATA |
| 30 | | GLSTVLFDKGQICGACYEVRCVEDL |
| | | RWCIPGTSIIVTATNFCAPNYGFDL |
| | | DGGGHCNPPNAHFVLPIEAFEKIAI |
| | | WKASNMPIQYRRIKCRKEGGVRFTL |
| | | GGSGIFLSVLISNVAGSGDIVAAKV |
| | | KGSRTGWLPMGRNWGQNWHINADLK |
| | | NQPLSFEMTSGDGATLTSYNVAPKN |
| | | WNFGQTFEGKQFGS* |
| SEQ | Solyc02g084720.3 | MEVNSLQKWVLLWCIVLFISSGLVH |
| ID | | CDVTYDRKAIVINGQRRLLFSGSIH |
| NO: | | YPRSTPEMWEDLINKAKEGGLDVVE |
| 31 | | TYVFWNVHEPSPGNYNFEGRYDLVR |
| | | FVKTIQKAGLYAHLRIGPYVCAEWN |
| | | FGGFPVWLKYVPGISFRADNEPFKN |
| | | AMKGYAEKIVNLMKSHNLFESQGGP |
| | | IILSQIENEYGPQAKVLGAPGHQYS |
| | | TWAANMAVGLDTGVPWVMCKEEDAP |
| | | DPVINTCNGFYCDNFFPNKPYKPAI |
| | | WTEAWSGWFSEFGGPLHQRPVQDLA |
| | | FAVAQFIQRGGSFVNYYMYHGGTNF |
| | | GRTAGGPFITTSYDYDAPIDEYGLI |
| | | RQPKYGHLKELHRAVKMCEKSIVSA |
| | | DPAITSLGNLQQAYVYSSETGGCAA |
| | | FLSNNDWKSAARVMFNNMHYNLPPW |
| | | SISILPDCRNVVFNTAKVGVQTSKM |
| | | EMLPTNSEMLSWETYSEDISALDDS |
| | | SSIRSFGLLEQINVTRDTSDYLWYI |
| | | TSVDIGSTESFLHGGELPTLIVETT |
| | | GHAMHVFINGQLSGSAFGTRKNRRF |
| | | VFKGKVNLRAGSNRIALLSVAVGLP |
| | | NIGGHFETWSTGVLGPVAIQGLDHG |
| | | KWDLSWAKWTYQVGLKGEAMNLVST |
| | | NGISAVDWMQGSLIAQKQQPLTWHK |
| | | AYFNTPEGDEPLALDMSSMGKGQVW |
| | | INGQSIGRYWTAYATGDCNGCQYSG |
| | | VFRPPKCQLGCGEPTQKWYHVPRSW |
| | | LKPTQNLLVLFEELGGDPTRISLVK |
| | | RSVTNVCSNVAEYHPNIKNWQIENY |
| | | GKTEEFHLPKVRIHCAPGQSISSIK |
| | | FASFGTPLGTCGSFKQGTCHAPDSH |
| | | AVVEKKCLGRQTCAVTISNSNFGED |
| | | PCPNVLKRLSVEAHCTPTQN* |
| SEQ | Solyc02g087530.2 | MSTLICLASPSTLVLSISKLKNPKT |
| ID | | ASLSVLCWTKEKKSLMNSKELHSAG |
| NO: | | ALTLKRSMIILRGVKKKTQPSQKKS |
| 32 | | YIITFNKQHREIVIGKYLKHVIEEG |
| | | RAIEFKNKKQKIYCNDRMPSWWACY |
| | | NSMWKHINFEHPATFDTLAMDPKKK |
| | | AEIIDDLIALTNGKDYYSKVGKAWK |
| | | RGYLLYGPPGTGKSTMIAAIANYLN |
| | | YDIYDLELTSVKDNLELKKLLMEIT |
| | | SKSIIVIEDIDCSVDLTGKRKKAKK |
| | | NKKKDDSDSDSDSDSDSDSDSDYEG |
| | | KKKLTLSGLLNFIDGIWSACGEERI |
| | | IIFTTNHVDKLDPALIRRGRMDMHI |
| | | EMSYCRYEAFKVLAKNYLGIDTHPL |
| | | FQEIQPLLEEVNLSPCDVAETLILK |
| | | NTSGGIEICLNNLILALKDSKEKAI |
| | | QEAREKEKQDSEEKPKKNKKLKKIY |
| | | GRFKKLFR* |
| SEQ | Solyc02g087770.3 | MSLKINLLICFFILHLWAATGHKIG |
| ID | | IYEIKKGDFSVKVTNYGARIISVLL |
| NO: | | PDKNGKIGDIVLGYDTIGEYVNDTS |
| 33 | | SFGAIVGRVVNRIAGARFTLNGTLY |
| | | KLIANDGKNTIHGGPKGFSHVVWKV |
| | | SKYKKDGPHPHITLTYHSTDGEQGF |
| | | PGDLLASVTYALEDPYKLSVVMKAK |
| | | ALNKATPVNLAQHTYWNLGGHNSGD |
| | | VLSNDIQIFASHITPLDQHFIPTGK |
| | | IAPVKNTPYDFLKPRKIGSQFKKLQ |
| | | NGYDINYPLDSTEKLKPTATVHDNK |
| | | SGRVMSLQTTAPAVQLYTANTLVKK |
| | | GKGGFVYQPYSAVCLETQGFPDAVN |
| | | HPNFPSTIVTPEHPYFHKMLFTFSI |
| | | KKY* |
| SEQ | Solyc02g089070.3 | MTTVVPTSEEDPVLSVVRFTAEMSW |
| ID | | ADAGAETAEPEVNRLCVEAQKCMIE |
| NO: | | GRWLDLVSLMLASADIIFSKASEKD |
| 34 | | LECVFTIMCNLVKKPESLDQVHEMA |
| | | ELISAKIIQQPNDKPALRLKILFNL |
| | | YNLLENPYSRFCVYLKSLKLATAGK |
| | | VTEHILSSIKMMDNFLKEWNLGVKD |
| | | QRELFLAISNILKESKGSTKDSFMF |
| | | LTKYLETFSSEDASNMTEAKEEAAS |
| | | AIVDFVRSPDMFKCDLLDMPAIVQL |
| | | EKDGQYGPVYQLLKIFLTQRLDAYL |
| | | EFEAANSALLKTYGLVHDDCISKMR |
| | | LMSLVDLGLNESSQIPYSVIKQALQ |
| | | IDDIEVESWVVKAITAKLLDCKIDQ |
| | | MNQVVIVSRCTERVFGVHQWQDLRT |
| | | KLVTWRGNIAGVISTVQANKITEDS |
| | | TQAVQGLAIR* |
| SEQ | Solyc02g092750.3 | MLSSCFGSCFGGDDGDDLLWHMDLK |
| ID | | PHATGEYSVAVVQANSLLEDQGQVF |
| NO: | | TTPSATYVGVYDGHGGPQASRFINN |
| 35 | | NLFPQLQKLALEEGGLSEDVIRKAF |
| | | DAIEEGFLRLVKQSWLDQPQIASAG |
| | | SCCLFGAISKDELYVANLGDSRAVL |
| | | GRRAETGQTGDSAVVVAERLSIDHN |
| | | VGVEEVRKEVEALHPDDSHVVVYVR |
| | | GVWRIKGIIQVSRSIGDVYLKKPEF |
| | | NRDPMFIQYGYPIPLKRAVMSAEPS |
| | | ILIRKIRPEDLFLIFASDGLWDQLT |
| | | DDEAKDIVLKNPRIGIAKRLVRAAL |
| | | EGAAEKKEMRYEDIKRLEKGERRHV |
| | | HDDITVIVIYLDHPGRSSNSGINIV |
| | | KGTNVPLDIYSLNSGH* |
| SEQ | Solyc03g007240.3 | MDGKENNGVNGSNKVGIPKCCLKAR |
| ID | | VFDPELEANCHSTVVSGWFSQPPTS |
| NO: | | FDGKEKVLYFNNPMWPGEAHSLKVE |
| 36 | | KVLFKGKSEFQEVLVFESASYGKVL |
| | | VLDGIVQLTEKDECAYQEMIAHLPL |
| | | CSIKSPKKVLVVGGGDGGVLREISR |
| | | HSSVELIDICEIDKMVIDVSKKYFP |
| | | DLAIGFEDPRVNLHVGDAVEFLKNT |
| | | PEGKYDAIIVDSSDPVGPAIELVEG |
| | | PFFAKIARALRPGGVLCNMAESMWL |
| | | HTHLIQDMISICRETFSSVHYAWAS |
| | | VPTYPSGVIGFLLCSTEGPFVDFKH |
| | | PVNPIEKLEGALQHQRELKFYNSEM |
| | | HEAAFALPCFLRREVSGLRDSPRSA |
| | | GSGVKI* |
| SEQ | Solyc03g080100.4 | MGEETKQEPPKEEVKTEEEQTEPKP |
| ID | | PSPFVLYVDLHCVGCAKKIERSISK |
| NO: | | IRGVEGVVIEMAKNTVTIKGVIEPQ |
| 37 | | AVCDRITKKTKRVAKVLSPLPAAEG |
| | | EPIPQVVASQVSGLTTVELNVNMHC |
| | | EACAEQLKKKILRMKGVRSAETEAS |
| | | TGKVTVTGTMDANKLVDYVYRRTKK |
| | | QAKIVPQPEPEPEKPVEETKPDEAK |
| | | PVEEPTPEDKKDEGTPPTENKEGGD |
| | | GEKPPEGEKNEGGGGGEAMIIPIED |
| | | HYDEHTINKLMYYHPYQPLYVIERI |
| | | PPPQLFSDENPNACCIT* |
| SEQ | Solyc03g111010.4 | MAKVKIGINGFGRIGRLVARVALQR |
| ID | | NDVELVAVNDPFISVDYMTYMFKYD |
| NO: | | SVHGQWKHHELKVKDEKTLLFGEKA |
| 38 | | VTVFGFRNPEEIPWAQTGADYIVES |
| | | TGVFTDKDKAAAHLKGGAKKVIISA |
| | | PSKDAPMFVVGVNEKEYKPELNIVS |
| | | NASCTTNCLAPLAKVINDRFGIVEG |
| | | LMTTVHSITATQKTVDGPSAKDWRG |
| | | GRAASFNIIPSSTGAAKAVGKVLPA |
| | | LNGKLTGMAFRVPTVDVSVVDLTVR |
| | | LEKEATYDEIKAAIKEESEGKLKGI |
| | | LGYTEDDVVSTDFVGDNRSSIFDAK |
| | | AGIALSKNFVKLVAWYDNEWGYSTR |
| | | VVDLIIHMSSVQ* |
| SEQ | Solyc03g112080.1 | MGYANGLLCPLGKQPLLSFGVISDV |
| ID | | QYADIDDGTSFLGVPRYYRHSVSVL |
| NO: | | QRAVKKWNQEKPKFVLNFGDIVDGY |
| 39 | | CPKDQSMIAVKKIVDEFDKFNGTVY |
| | | HMIGNHCLYNLPRKDLLPLLRIPGH |
| | | DDHAYFDFSPIPEYRFVILDAYDIS |
| | | AIGWPEDHPNTLKALKVLQEKNPNS |
| | | DKNSPSGLVGPARRFLMFNGGVGKE |
| | | QLEWLDHVLQEATKLNQNVIVCCHL |
| | | PLDPGASSLAALLWNYDEVMDVIHR |
| | | YSCVKVCMGGHDHKGGQSVDSHGVH |
| | | HRVLEAALECPPGTDSFGHIDAFDD |
| | | RLLLFGTDRMKSTEMVFRH* |
| SEQ | Solyc03g112620.4 | MNTSSSIIFKFVAFLVIFSSFSTHV |
| ID | | LSTLQANQTFRPKEELLKLKKIKGY |
| NO: | | LKKINKRPVRTIKSPDGDLIDCVLS |
| 40 | | HLQPAFDHPQLKGMKPLEPPKRPKA |
| | | NESMNIMEESLQLWTVSGESCPEGT |
| | | VPIRRTTEKDVLRSNSLRRFGRKIR |
| | | RGVRHDTSSNDHEHAVAFVNGEQYY |
| | | GAKASINVWTPRVTDQYEFSLSQLW |
| | | VISGSFGNDLNTIEAGWQVSPELYG |
| | | DNYPRFFTYWTTDAYQATGCYNLLC |
| | | SGFVQINNRIAMGAAISPRSSVNGR |
| | | QFDIGIMIWKDPKHGHWWLEFGSGL |
| | | LVGYWPDFLFSHLRGHASMVQFGGE |
| | | VVNSRSMGYHTSTQMGSGHFADEGF |
| | | GKASYFRNLQVVDWDNNLIPLSNLH |
| | | LLADHPNCYDIRAGKNNVWGNYFYY |
| | | GGPGRSSRCP* |
| SEQ | Solyc04g005040.1 | MRRIHLYIAIAYVVIFQTSCSAHFF |
| ID | | PNISSIPSSLLKPNATAWTSFQKLL |
| NO: | | GCQPGQKVDGIAKIKKYFQHFGYIN |
| 41 | | NLTSFNFTDEFDDTLESALKTYQRN |
| | | FNLKATGVLDAPTIQHLIKPRCGNA |
| | | DLVNGTSTMNAGKPHTVAHYSFFPG |
| | | RPKWPEGKTDLTYAFLPANNLTDDI |
| | | KSVFSRAFDRWSEVTPLSFTEIPSF |
| | | QSADIKIGFLTGDHNDGEPFDGPMG |
| | | TLAHAFSPPAGHFHLDGEENWVVDG |
| | | VPVNEGNFFSILSAVDLESVAVHEI |
| | | GHLLGLGHSSVEDSIMYPSLESGIR |
| | | RVELVEDDIKGVQELYGSNPNYTGT |
| | | NTTLTPSGLDNDTNGAPIRSSVWIH |
| | | GFLLVVGFFIYSI* |
| SEQ | Solyc04g014510.3 | MAHLSDLVNLNLSDSSEKIIAEYIW |
| ID | | IGGSGMDVRSKARTLSGPVDDPSKL |
| NO: | | PKWNYDGSSTGQAPGEDSEVILYPQ |
| 42 | | AIFKDPFRRGNNILVICDCYTPAGE |
| | | PIPTNKRHNAAKIFSNPNVVVEEPW |
| | | YGLEQEYTLLQKEINWPLGWPIGGF |
| | | PGPQGPYYCGIGCGKAFGRDIVDAH |
| | | YKACIYAGINISGINGEVMPGQWEF |
| | | QVGPSVGIASGDELWAARYILERIT |
| | | EIAGVVVSFDPKPIPGDWNGAGAHT |
| | | NYSTKSMRNEGGYEVIKKAIEKLGL |
| | | RHKEHIAAYGEGNERRLTGRHETAD |
| | | INTFKWGVANRGASIRVGRDTEKEG |
| | | KGYFEDRRPASNMDPYVVTSMIAET |
| | | TILWNP* |
| SEQ | Solyc04g014570.3 | MCTKFVKEMEKMALYVVVLLIICYA |
| ID | | SPARSDGSDHKYKSGDQVPLYANKV |
| NO: | | GPFHNPSETYRFFDLPFCTPDHVKE |
| 43 | | KKEALGEVLNGDRLVSAPYKLDFLY |
| | | DKDSEIVCKKKLSKEEVSQFRSAVA |
| | | KDYYFQMYYDDLPIWGFLGKVDKEG |
| | | KSDPSEYKYYLFKHLHFEIFYNDDR |
| | | VIEINARTDPNALVDITEDKEVDVD |
| | | FMYTVKWKETKTPFDKRMEKYSQSS |
| | | SLPHHLEIHWFSIINSCVTVLLLTG |
| | | FLATILMRVLKNDFVKYAHDEEAAD |
| | | DQEETGWKYIHGDVFRYPSHKSLMA |
| | | AALGSGTQLFTLTIFIFLLSLVGVF |
| | | YPYNRGALFTALVVIYALTSGIAGY |
| | | TAASFYCQLEGTNWVRNLILTGALF |
| | | CGPLFLTFCFLNSVAIAYSATAALP |
| | | FGTIVVIFLIWALVTSPLLVLGGIA |
| | | GKNSRAEFQAPCRTTKYPREIPPLP |
| | | WYRGTLPQMAMAGFLPFSAIYIELY |
| | | YIFASVWGHRIYTIYSILFIVFIIL |
| | | IIVTAFITVALTYFQLAAEDHEWWW |
| | | RAFLCGGSTGLFIYGYCLYYYYARS |
| | | DMSGFMQTSFFFGYMACICYGFFLM |
| | | LGTVGFRAALFFVRHIYRSIKCE* |
| SEQ | Solyc04g050990.3 | MAVERFACGDELPEGCIATALSLTS |
| ID | | PKDACRLALVASTFRSAAESDAVWE |
| NO: | | RFLPPDYYDIISRSIDGPDSLNFGS |
| 44 | | KKDLYLYLCDNPILIDGGTRSFSLE |
| | | KGSGKKCYMLAARSLQIVWVDTPRH |
| | | WRWISLPESRFSEVAELLDVCWFDI |
| | | SGKINTNMLSPDTTYGAYLVFTTKT |
| | | RTCGFENQPAESSVGIAGHERYTHT |
| | | FYLNPQGGRRQYRYQVVPRRLGIFT |
| | | HHMARILREETEDAPKGCRPKQRSD |
| | | GWMEIELGEFFVKRGQEAEVEISLT |
| | | EVKGGDRKAGLIIEGIDIRPKE* |
| SEQ | Solyc04g071890.3 | MVSTTLSSFLLVLVLSFFVSEAQRP |
| ID | | ALTKGLSWSFYQSSCPQLESIIRNR |
| NO: | | LQKQIKDDVGQAAGLLRLHFHDCFV |
| 45 | | QGCDGSVLLDGSAGGPSEQTAIPNL |
| | | TLRKRSFKIIDDLRKRIQDECGQVV |
| | | SCSDIVAIAARDSVVLTGGPNYDVP |
| | | LGRKDGVNFATEQATIDNLVAPSAN |
| | | TTTVLSLLATKGLDATDAVALSGAH |
| | | TIGISHCPSFTDRLYPNQDSTMDKT |
| | | FANNLKGSCPTADSNNTVNMDIRSP |
| | | NVFDNKYYVDLMNRQGLFTSDQDLY |
| | | TDRRTRGIVTSFAVNQSLFYEKFVI |
| | | GMIKMGQMNVLTGGQGEIRNRCDRR |
| | | NKAKKVDIATVVEELEETFSALF* |
| SEQ | Solyc04g077190.4 | MKRLCAFSFARRLFKPRTTHSQESK |
| ID | | EKMEQSPIVNVEDNFDLQESKESME |
| NO: | | KSPTVTANNNFDTQSATENEKENGS |
| 46 | | YAATNIILNHEFSDGLHSWHPNCCD |
| | | AFVVPAGSGYHKGLAAKEGCCYAVV |
| | | SNRKECWQGLEQDITSRVSAGSTYT |
| | | VSACVGATGTFQGSVEVLATLKLVY |
| | | QNSETSYLFVAKKAASEECWETLEG |
| | | SFSLSTMPDQVIFYLEGPPAGTDLL |
| | | IKSVVISCPSSTASDSSGTSSVYID |
| | | DDNIIINPQFDDGLNSWSGRGCKVA |
| | | LHDSMADGKITPMSGKSFASATERT |
| | | QSWNGIQQDVTGRVKRKLAYEVSAV |
| | | VRIYGNNVTTADLRSTLYVKAADNR |
| | | ERYIGIASVQATDKDWVKLQGKFLI |
| | | NDSPSQVVVFLEGPPAGTDILLNNL |
| | | VIKHAAKAPPSSPPVIEDAGFGVNI |
| | | ITNTSLNDGTNGWFPLGNCTMSVQT |
| | | GSPHIMPPMARDTLGAHEPLSGRYI |
| | | LVANRTQNWMGPAQMITEKVKLYLT |
| | | YQVSAWVKIGQTSGPQNVNVALGVD |
| | | SQWVNGGQAEISDDRWHEIGGSFRI |
| | | EKQAAKIMVYIQGPVAGVDLMVAGL |
| | | QIFPVDRRERFRHLKKQTAKLRKRD |
| | | VMLKFSGSDSGNLFGTFVRVKQLQN |
| | | SFPFGSAISRTNMDNEDFNAFFVKN |
| | | FNWAVFGNELKWYWTEAQQGNLNYK |
| | | DADELLDFCTKNNIQVRGHCIFWEV |
| | | VGTVQAWVQSLNKNDLMTAVQNRLT |
| | | GLLTRYKGKFPHYDVNNEMMHGSFY |
| | | QDRLGKDIRVNMFKTAHQLDPSPIL |
| | | FVNDYHVEDGCDTRSYPEKYIEHIL |
| | | DLQEHGAPVGGIGIQGHIDSPVGPI |
| | | VCSALDKLGTLGLPIWFTEVDVSSD |
| | | NEYVRADDLEVMLREAYAHPAVEGI |
| | | MLWGFWELFMSRTNAHLVDAEGDIN |
| | | EAGKRYLALKHEWSSHPHGHIDEQG |
| | | QFSFSGFHGSYEVEVVTVSKKITKK |
| | | FVVDKGDNALSSKENGNIILNHEFL |
| | | DGLHLWRPNCCHAFVVPAGSGYRNG |
| | | LTMAVVTKSTIWWQGLEQDITSRVS |
| | | AGSSCTASDESKQEMEKAPPTITLI |
| | | LSSKEDEKDTGNIILNHDFSYGLYL |
| | | WNPNCCEAFVVPSGYHKGLAAAIVT |
| | | NRRECWHGLEQDITSKVSEGSTYTV |
| | | SACVGASGTCPGSVELTCS* |
| SEQ | Solyc04g078390.3 | MKRQFDAGETDWANFTGGDHVKKMK |
| ID | | KEMLELEEERKLPMKRQFEANCAAK |
| NO: | | VSGDLKKMKQVIESPDADADADTEA |
| 47 | | VLLNENLLYEVLKHVDGRTLATAAC |
| | | VSKQWNQTALDERLWELICTRHSCN |
| | | QQQQLRAVVLALGGFRRLYSLYLWP |
| | | LSKPSPSPSASSSNPPASAWPCLPP |
| | | APTRPLKYAAANTRWGKDEVNLSLS |
| | | LLSIRYYEKMNFKANK* |
| SEQ | Solyc04g078540.4 | PSSFSIPQLKQSNMAIHLSALTISI |
| ID | | LVMVVSSAAVTSAAAEDMSIISYNE |
| NO: | | KHHTIGAGRTDDEVMSMYESWLVEH |
| 48 | | KKVYNALGEKDKRFQIFKDNLKYID |
| | | EHNAMPEKSYKLGLTKFADLTNEEY |
| | | RSVYLGTKPDASRRLSSRQSDRYAP |
| | | KVGDRLPESVDWVKKGVLVGVKDQG |
| | | QCGSCWAFSAVAAIEAVNKIKTGDS |
| | | ISLSEQELVDCDTSSNNGCDGGLMD |
| | | YAFEFVIKNGGLDTEEDYPYTGEDG |
| | | RCDLTRKNAKVVTIDGYEDVPANDE |
| | | NAMKKAIASQPVSVAIEAGGKDFQH |
| | | YKSGIFTGKCGAAVDHGVVAVGYGS |
| | | ENGMDYWIVRNSWGASWGEHGYLRM |
| | | QRNIANPKGLCGIATVVSYPVKTGQ |
| | | NPPKPAPSPPSPVKPPTTCDDMYSC |
| | | PSGTTCCCVYEYYHMCFAWGCCPME |
| | | GATCCKDHNSCCPHDYPVCNVKAGT |
| | | CSISENNPLSVKAMSHILAKPIGSF |
| | | SNQGMKNTIS* |
| SEQ | Solyc04g078760.4 | MALPHHHLQLHIQQQPHQQQQQSKS |
| ID | | YRDLYNNMDGQITNPVVYFNGSNLP |
| NO: | | EQSQHPPYIPPFQVVGLAPGTADDG |
| 49 | | GLDLQWNYGLEPKKKRPKEQDFMEN |
| | | NNSQISSVDLLQRRSVSTGLGLSLD |
| | | NGRLASSCDSAFLGLVGDDIERELQ |
| | | RQDAEIDRYIKVQGDRLRQAVLEKV |
| | | QANQIQAITYVEEKVLQKLRERDTE |
| | | VDDINKKNMELELRMEQLALEANAW |
| | | QQRAKYNENLINTLKVNLQHVYAQS |
| | | RDSKEGCGDSEVDDTASCCNGRATD |
| | | LHLLCRDSKEMKELMTCRVCRTNEV |
| | | CMLLLPCKHLCLCKECESKLSLCPL |
| | | CQSTKYIGMEVYM* |
| SEQ | Solyc04g080190.3 | MKASFKARYEPDKAAANATVAFNAG |
| ID | | DLKLRASMTDATVVKGPSLNGLALA |
| NO: | | VEKPGLFIVDYNVPKKDIRFQFMNS |
| 50 | | IKVLEKPLNLNYIHFHGDKRTILDG |
| | | TLVVDSANKVSANHVMGSGSCKLKY |
| | | TYVHGGITTFEPIYDTAKNAWDFMV |
| | | SRKVYGDDVFKATYQTTSKNLGLEW |
| | | SRSSKLNGSFKICASLCLLEERKIP |
| | | KLSAETSWDFEM* |
| SEQ | Solyc04g080960.4 | MLNAEHHFSLFKAKFGKIYASQEEH |
| ID | | DHRLKVFKANLHRAKRHQLLDPSAE |
| NO: | | HGITQFSDLTPSEFRRTYLGLNKPR |
| 51 | | PNLNAEKAPILPTKDLPSDFDWREK |
| | | GAVTDVKNQGSCGSCWSFSTTGAVE |
| | | GAHFLATGELVSLSEQQLVDCDHEC |
| | | DPVEKNDCDAGCNGGLMTTAFEYTL |
| | | KAGGLQLEKDYPYTGRNGKCHFDKS |
| | | RIAASVSNFSVVGLDEDQIAANLLK |
| | | HGPLAVGINAAWMQTYVRGVSCPLI |
| | | CFKRQDHGVLLVGYGSEGFAPIRLK |
| | | NKPYWIIKNSWGKTWGEHGYYKICR |
| | | GHNICGVDAMVSTVTATHTTNPNL* |
| SEQ | Solyc04g082030.1 | MAGQTVIVSGLNPAAILQSTIGGAP |
| ID | | VAAAAENGHTRKVVPLSKDALQDFM |
| NO: | | VSIITQKLQDDKQPFYVLDLGEVVS |
| 52 | | LMEQWNSALPNIRPFYAVKCNPEPS |
| | | FLSMLSAMGSNFDCASRAEIEYVLS |
| | | HGISPDRIVFANPCKPESDIIFAEK |
| | | IGVNLTTYDSEDEVYKIRKHHPKCE |
| | | LLLRIKPMTDGNARCPMGPKYGALP |
| | | EEIEPLLRTAQAARLTVSGVSFHIG |
| | | SGDADSNAYLGAIAAAKQVFETAAQ |
| | | LGMPKMTVLDIGGGFTSGHQFTTAA |
| | | PAVKSALETHFHDFPELTIIAEPGR |
| | | FFAETAFTLATTIIGKRVRGELKEY |
| | | WINDGLYGSMNCVLYDHATVTATPL |
| | | ACMSNRNNLNCGGSKTFPSTVFGPT |
| | | CDALDTVLRDYQLPELQVNDWLIFP |
| | | NMGAYTKAAGSNFNGFNTSAIVTHL |
| | | AYAYPN* |
| SEQ | Solyc04g150161.1 | MAKIAYLLTTLLVALVSLSFPSECK |
| ID | | ANYYYTSPPPPTPVYKYKSPPPPVY |
| NO: | | KYKSPPPPPPVYKSPPPPVYKYKSP |
| 53 | | PPPVYKYKSPPPPPPVYKYKSPPPP |
| | | VYKYKSPPPPPPVYKYKSPPPPVYK |
| | | YKSPPPPPPVYKYKSPPPPVYKYKS |
| | | PPPPPPVYKYKSPPPPVYKYKSPPP |
| | | PPPVYKYKSPPPPVYKYKSPPPPTP |
| | | VYKYKSPPPPVYKYKSPPPPTPVYK |
| | | YKSPPPPVYKYKSPPPPTPVYKYKS |
| | | PPPPVYKYKSPPPPTPVYKYKSPPP |
| | | PVYKYKSPPPPTPVYKYKSPPPPVY |
| | | KYKSPPPPVYKYKSPPPPVYKYKSP |
| | | PPPVYKYKSPPPPVYKYKSPPPPVY |
| | | KYKSPPPPVYKYKSPPPPVYKYKSP |
| | | PPPVYKYKSPPPPPPVYKYKSPPPP |
| | | VYKYKSPPPPVYKYKSPPPPVHKSP |
| | | APYYYTSPPPPSHY* |
| SEQ | Solyc05g005200.3 | MGSEGPKSVTIHVTGFKKFHGVAQN |
| ID | | PTETAVSNLKYYVEKKGLPAGVTLG |
| NO: | | SCTVLETAGEGGLPTLLKVIEASSA |
| 54 | | SNSSNNGQVIWLHLGVNSGATKFAI |
| | | ERLAVNEATFRCADELGWQPQRVPI |
| | | VPEDGGINQIRKTCCPTESILELLK |
| | | KKGFDVTLSDDAGRFVCNYVYYHSL |
| | | RFAEEKGHKCLFVHVPTFHRVHQEK |
| | | QMEFVAALLEAIASTC* |
| SEQ | Solyc05g015630.3 | MDPNDLAKAFVEHYYSTFDSNRAAL |
| ID | | GNLYQEGSMLTFEGIKTQGSQNIVA |
| NO: | | KLTSLPFQQCHHTITTIDCQHSGPA |
| 55 | | GGMIVVVSGNLQLAGEQHALKFGQM |
| | | FHLMPTPQGSFYVFNDIFRLNYG* |
| SEQ | Solyc05g050800.3 | MAANQNVIVMRHGDRLDNFEELWVM |
| ID | | KAERPWDPPLHQDGKIRAFCTGQKI |
| NO: | | RSSIDFPIHRVFVSPFLRCVQTAAE |
| 56 | | VVRALCDVADHNGESNVLSSNSDSV |
| | | IIDPSKVKVSIEYGLCEMLNLVAVR |
| | | ASAAPKDGDFKFSISQYESELPAGT |
| | | VDNTVEPVYKKLPEWEESLESARAR |
| | | YVKVVKALADKYPSENLLLVTHGEG |
| | | VGSIFTELNKDSTVLEVAYCGHLHV |
| | | KRSIQSGEKQPFTAGEFVYEKQTGI |
| | | ISAAK* |
| SEQ | Solyc05g056250.3 | MESEKPQNPSPMEPQEEGSNKISKK |
| ID | | EAAKLERQRRRQEAAAAAAATAVST |
| NO: | | VSIEESDPLSGNYGDVELNDLQSKA |
| 57 | | VTGRKWTEVGSIAPEMKDRVVLVRG |
| | | RVQTIRPVGKKIAFVVVRERGFTVQ |
| | | CVLTVKPELVSSQMVKYATSLSKES |
| | | IIDVEGVVTIPEKPITGATQQQVEV |
| | | QISKLYCVNRAVPTLPINIEDAARS |
| | | EVEIEQALEKGEQLVRVNQDTRLNF |
| | | RILDMRTPANQGIFRIQCQIENIFR |
| | | QFLLSEGFVGIHTPKLIGGSSEGGS |
| | | AVFRLDYKGQPACLAQSPQLHKQMA |
| | | ICGDFGRVFEVGPVFRAEDSFTHRH |
| | | LCEFTGLDVEMEIKEHYSEVMDIVD |
| | | RLFVAMFDSLNEKCKKELEAVGKQY |
| | | PFEPLKYLRKTLRLTFAEGIQMLKE |
| | | AGVEVDPLGDLNTESERKLGQLVAE |
| | | KYGTEFYILHKYPQAVRPFYTMPCH |
| | | DNPSYSNSFDVFIRGEEIISGAQRV |
| | | HVPELLEKRAGECGIDVKTISTYID |
| | | SFRYGAPPHGGFGVGLERVVMLFCA |
| | | LNNIRKTSLFPRDPQRIVP* |
| SEQ | Solyc05g056340.3 | MSGTPNKRPHEDGGNGGSSNHSYSS |
| ID | | APKYSHDDSGAFPKVMSSGTPEYHA |
| NO: | | SFDVGQNARMPKIQRTESSRDADRR |
| 58 | | SPVLPMYRVSSCPVVSHPDHSVASE |
| | | NRLEPKEVNKDVKVENRDAKSEIRE |
| | | LYQGTKSDKDDRFENRADDGKDIKN |
| | | SRDTYPEYKGDVKTDKDRFSGVSWK |
| | | DPKEQTRGKRYPDLPVPVGNMDPWH |
| | | ASRTHGAAEIGKEVSNSENRDFAKV |
| | | REAVAENKMDLKGDDKYKDKERKRK |
| | | EGKHREWGERDKERNDCRNNLQLGN |
| | | STSDNKELLKEERESERWEKERNDL |
| | | SKDKDRPKDWEKDHAKREVWNGVER |
| | | EVLQSEKEVIDVPGKTNEPENSTVE |
| | | QKKQKDHDNWKNTDRDGSERRKERD |
| | | TDLEGERPEKRVRCHDKEPEEGDLD |
| | | TEGGGEREREAFNYGVQQRKRMSRP |
| | | RGSPMANRDPRFRSHTHENEGSQVK |
| | | HDVSAVNYRVGECMPELIKLWKEYE |
| | | SSKADEASDSSPSDPTLEIRIPAEH |
| | | VSATNRQVRGGQLWGTDIYTNDSDL |
| | | VAVLMHTGYCRTTASPLLPTITELR |
| | | ATIRVLPPQNCYISTLRNNVRSRAW |
| | | GAAVGCSYRIERCSVVKKGGGTIDL |
| | | EPCLTHSSTLEPTLAPVAVERTMTT |
| | | RAAASNALRQQRFVREVTIQFNLCN |
| | | EPWLKYSISVVADKGLKKALFTSSR |
| | | LKKGEVLYLETHSKRYELCFSGEKM |
| | | VKATTSLMHEMDVDKPQSHNLHMAN |
| | | GEKNGVNGENTMVDMFRLSRCKKPL |
| | | PQKLMQSVGIPLPLEHVEVLEENLE |
| | | WENIQWSQTGVWIAGKEYPLTRAHF |
| | | LSPN* |
| SEQ | Solyc06g008340.4 | MSFDEDEESFEHTLLVVREVNVFKI |
| ID | | PPRPTSGGYKCGEWLQTDKIWSGRL |
| NO: | | RVVSCKDRCEIRLEDPNSGELFAAC |
| 59 | | FVPPGQRENSVESVLDSSRYFVLKI |
| | | EDGTGKHAFIGLGFSERNEAFDFNV |
| | | ALSDHEKYVKRDIEKDGDEAETSSD |
| | | GHIDIHPAVNHRLKEGETIRINVKN |
| | | KSSSGMGMLSAAVQSKAITIAPPPS |
| | | GANKIRSPLPPPPNDPAISRKTSTT |
| | | ASIALKGPNGSSMQSSDPISDLSQL |
| | | ERSLPSATGSGSSKKTTAAGWAAF* |
| SEQ | Solyc06g050770.3 | MGDQIARGEDFESKAEKKLGGWAIF |
| ID | | GSKYEDAADLFDKAGNCFKLAKSWD |
| NO: | | KAGAVYVKLANCHLKLDSKHEAANA |
| 60 | | YADAAHCYKKSNIKEAVSCLEQAVN |
| | | LFLDIGRLNMSARYYKEIAELYEQE |
| | | QNLDQAINYYEKAADLFQSEDVTTT |
| | | ANQCKQKVAQFSAQIEKYPRAIEIF |
| | | EEIARHSVNNNLLKYGVRGHLLNAG |
| | | ICQLCKGDVVAINNALERYQELDPT |
| | | FSGTRECKLLVDLAAAIDEEDVAKF |
| | | TDAVKEYDSMTQLDAWRTTLLLRVK |
| | | ETLKAKELEEDDLT* |
| SEQ | Solyc06g053510.3 | MEVIMNNHNNGTINTNTKIIHKNGS |
| ID | | ICNGNGNVNGNSHTHNNENKLVEFT |
| NO: | | NSIKPGWFSEFSALWPGEAFSLKIE |
| 61 | | KLLFQGKSDYQDVMLFESATYGKVL |
| | | TLDGAIQHTENGGFPYTEMIVHLPL |
| | | GSIPSPKKVLIIGGGIGFTLFEVSR |
| | | YPSIEKIDIVEIDDVVVDVSRKFFP |
| | | YLAAGFDDPRVSLIIGDGAAFVKAA |
| | | QPGYYDAIIVDSSDPIGPAKDLFER |
| | | PFFEAVAKALRPGGVVCTQAESIWL |
| | | HMHLIKKIIANCRQVFKGSVNYAWT |
| | | TVPTYPTGVIGYMLCSTEGPEVNFK |
| | | NPVNSIDKYVKSKGPLKFYNSDIHK |
| | | SAFILPSFARDLIES* |
| SEQ | Solyc06g065100.3 | MGRSPCCEKAHTNKGAWTKEEDQRL |
| ID | | INYIRAHGEGCWRSLPKAAGLSRCG |
| NO: | | KSCRLRWINYLRPDLKRGNFTEEED |
| 62 | | ELIIKLHSLLGNKWSVIAGRLPGRT |
| | | DNEIKNYWNTHIKRKLISRGIDPQT |
| | | HRPLNNNATNSHTTTNITTAVTTKN |
| | | INLDFTNVDQKQPNIMIATSSSYDE |
| | | TKCNSGTTEETKPLEIIIPKIPSQV |
| | | MINLELSIGLPLHTDHISSPESTAS |
| | | YNFLTTVAPPPTAAVPAAEMMAKTV |
| | | CLCWQIGYHGGGGQWCGKCKNINGF |
| | | YRYC* |
| SEQ | Solyc06g072120.3 | MATQDVRTLSTKEADIQMMLAAEVH |
| ID | | LGTKNCDFQMERYAFKRRNDGIYII |
| NO: | | NLGKTWEKLQMAARVIVAIENPQDI |
| 63 | | IVQSARPYGQRAVLKFAQYTGAHAI |
| | | AGRHTPGTFTNQLQTSYSEPRLLIL |
| | | TDPRTDHQPIKEAALGNIPTIAFCD |
| | | TDSPMRYVDIGIPANNKGKHSIGVL |
| | | FWILARMVLQMRGAINQGPKWDVMV |
| | | DLFFYREPEEAKEQEEEVPAIADYA |
| | | DYSASAALGGDWTSSQIPEAQWTAD |
| | | AAAPAVGGGWAGDGAADGGWDAAAA |
| | | PAPVPLPVPDVAPTSGATGWE* |
| SEQ | Solyc06g073150.3 | MDSLNSYASSAAMAQQTWELENNIV |
| ID | | TIDAPSGSKPENSASDAIFHYDDAA |
| NO: | | QTKFQREKPWASDPHYFKRVKISAL |
| 64 | | ALLKMVVHARSGGTIEVMGLMQGKT |
| | | DGDAIIVMDAFALPVEGTETRVNAQ |
| | | ADAYEYMVEYSQTNKQAGRLENVVG |
| | | WYHSHPGYGCWLSGIDVSTQMLNQQ |
| | | YQEPFLAVVIDPTRTVSAGKVEIGA |
| | | FRTYPEGYKPPDDPISEYQTIPLNK |
| | | IEDFGVHCKQYYSLDITYFKSSLDC |
| | | RLLDLLWNKYWVNTLSSSPLLGNGD |
| | | YVAGQISDLAEKLEHAENQLSNSRY |
| | | ASLMAPPQRKKEEESQLAKITRDSA |
| | | KITVEQVHGLMSQVIKDILFNSVGK |
| | | SSKSQTESSGPEPMIES* |
| SEQ | Solyc06g076410.4 | MASMIESAWTYLITNFNDFQLACLG |
| ID | | SFFLHESVFFLSGLPFILLERAGWL |
| NO: | | SKYKIQTKNNTPAAQEKCITRLLLY |
| 65 | | HFCVNLPLMMASYPVFKLMGMRNTL |
| | | PLPSWKVISTQVLFYFILEDFVFYW |
| | | GHRILHTKWLYKHVHSVHHEYATPF |
| | | GLTSEYAHPAEILFLGFATIVGPII |
| | | TGPHLITLWLWMIVRVLETVEAHCG |
| | | YHFPWSLSNFLPLYGGADFHDYHHR |
| | | LLYTKSGNYSSTFVYMDWLFGTDKG |
| | | YRRLKVLKSDACEAEGKEM*RCERV |
| | | SFGWSGIARRAYVQERIGEGFCC* |
| SEQ | Solyc06g082380.3 | METTATPEEWPPDGQFPFDLFVSKK |
| ID | | YKSFWDAGNIQFTDAVGNLFFSVDR |
| NO: | | RQPQNSAAQTHPQKLILDASDNTLI |
| 66 | | RLVRLTKGSWQGFMVSDNEEKALMF |
| | | SVNKTLNTFTKLEFDIFLGDGHVEG |
| | | AEADLKMKGSAFKRSCTIYKGNSIV |
| | | AETSLMYTLGFRKHFIPRNRFRVTI |
| | | FPGLTELSLVVALVVVFFDKQKFWL |
| | | * |
| SEQ | Solyc06g082670.3 | MGRRPARCYRQIKNKPYPKSRFCRG |
| ID | | VPDPKIRIYDVGMKKKGVDEFPFCV |
| NO: | | HLVSWEKENVSSEALEAARIACNKY |
| 67 | | MTKSAGKDAFHLRVRVHPFHVLRIN |
| | | KMLSCAGADRLQTGMRGAFGKPQGV |
| | | CARVAIGQVLLSVRCKDGNSNHAQE |
| | | ALRRAKFKFPGRQKIIVSRKWGFTK |
| | | FSRTDYLRYKSENRILPDGVNAKLL |
| | | GNHGRLAARQPGRAFLTSA* |
| SEQ | Solyc06g083210.3 | MERYRILKELGDGTCGNVYKAINTE |
| ID | | TSEIVAVKKMKRKFYFWEECINLRE |
| NO: | | IKSLRKLNHPNIIKLKEIVRENNEL |
| 68 | | FFILEYMECNLYQLMKDRQRPFLEE |
| | | EIRGLMSQVLQGLTHMHKNGYFHRD |
| | | LKPENLLVTNDVIKIADFGLAREVS |
| | | SSPPFTDYVSTRWYRAPEVLLQSSS |
| | | YTPSIDMWAVGAVLAELFTLCPIFP |
| | | GESEIDQLYKICCVLGPPDWTSFPE |
| | | LRSASRLFDINCFDITPANLSDFIP |
| | | NASLEAIDLIKQLCSWDPLRRPTAD |
| | | QCLQHPFFHVDMSIPRPLEDPLQMN |
| | | LSSVGPEPNLELNLWDFGTEKDDCY |
| | | LGLTLAVNPTPSCLDLFPAKMASKS |
| | | QGAGTDMFCSGFQDHSQHSVFWSLF |
| | | PTDRHQISTPVDSSLSLSFSTIPQS |
| | | TIGAPQSTSFGMTSLQSNFLERPFL |
| | | AMSSSFQQGRCL* |
| SEQ | Solyc06g083300.4 | MAAEAAVAVAAVAPVSAQVVANAFV |
| ID | | QQYYHILHHSPGLVFRFYQDISKLG |
| NO: | | RPEEDGSMGITTTMQAINDKILSLN |
| 69 | | YGDLRAEIKSVDAQESFNGGVHVLV |
| | | TGYLTGKDNLVRNFCQTFFLAPQDR |
| | | GYFVLNDMFRYVETASQHDTVQVPV |
| | | TDAVAPVTPEQIVADPPTAPQNHIP |
| | | EQSTPPAEEADVGEVYNPSENGDVL |
| | | IVEEEVPVSEVVHEVQDGSEVVAES |
| | | DNKTGEVPKKSYASIVMHLKESAAT |
| | | FSPPPAPAARKPMTKSVVQVNQPAA |
| | | TTTDGPYSSSETIDNINDTEADGYS |
| | | IYIKGLPMSATVALLADEFKKFGPI |
| | | KNGGIQVRSNRQQGFCFGFVEFEVE |
| | | SAVQKAIEASPVLIGDRQAVVEEKR |
| | | STNSRLNARGGGRFQSGRGGGFRND |
| | | VGGRGRGNYLGGRGYGRDDFHGRNE |
| | | FNNRGGNRGGSSNRGGEGYRRADNT |
| | | GGRMSRGGGMSNGTAKGPAPRYSAT |
| | | A* |
| SEQ | Solyc07g007930.3 | MQLSFSRPKSPMMFDLISISSHHPN |
| ID | | SIINTINRPIQNPKIPSFFSSTTTH |
| NO: | | FSYSFNKYSNPLQFSVSSPTTSVLA |
| 70 | | HKGSEVEFEKGLEEEEAAAKTVVDA |
| | | AMTITPAIRISDRKLMVKDRTILTN |
| | | VPDNVLTTPGAASGPLEGVFLGAEF |
| | | DQDNNRHVVPLGKLQDVRFLSCFRF |
| | | KLWWMAQKMGDKGSEIPMETQFLLV |
| | | ETTDGSHLGSNDNKNDDNIVYAVFL |
| | | PLIEGSFRAVLQGNAEDELELCLES |
| | | GDKDTVGSAFNQAVYIHAGSDPFIV |
| | | ITEAIRAVKLHLKTFRQRHEKKLPK |
| | | IVDYFGWCTWDAFYQEVTQEGVEAG |
| | | LKSLTAGGIPPKFIIIDDGWQSVGG |
| | | DPEVDKPLMRLTGLKENEKFQKKED |
| | | PTLGIKNIVNIAKEKYGLNYVYVWH |
| | | AITGYWGGVRPGVKGMEEYGSVVKY |
| | | PDITKGVMENEPGWKTDAIAVQGLG |
| | | LVNPKSAYKFYNEMHSYLASAGVDG |
| | | LKVDVQCILETLGGGLGGRVELTKQ |
| | | YHQALDASVARNFPDNGCIACMSHN |
| | | TDALYCSKQTAVVRASDDFYPRDPA |
| | | SHTIHIACVAYNSVFLGEIMLPDWD |
| | | MFHSLHPAAEYHGSARALSGGPVYV |
| | | SDAPGKHNFDVLRKLVLPDGSILRA |
| | | RLPGRPTKDSLFTDPSRDGVSLLKI |
| | | WNMNKYTGVLGIYNCQGAAWSTVER |
| | | KTTFHKTNSEAITGYIRGCDVHFIS |
| | | EAALDPNWSGDTVLYSHGSAELVVL |
| | | PYNAAMPVSFKILEHETYTVTPIKV |
| | | LAPGFSFAPLGLIDMYNAGGAIEGL |
| | | KYEVKAGAELSELEAGYQGEGNLVA |
| | | EDKIENLSTEAVAVVSMEVRGCGRF |
| | | GVYSSVKPRKCSVGGDMVDFAYNSE |
| | | SGLLTLNLDAMPPADQKVHIIEVEV |
| | | * |
| SEQ | Solyc07g015980.4 | MASTCLDVSFLGNFCNNWSFRPCTR |
| ID | | LTPCISQVRNSLLLGKNKNYTSSVG |
| NO: | | YKGIPLTEISCVPSAVASSSELPPL |
| 71 | | VSALKASAEENAASFHFPGHNRGQA |
| | | APSSLTQLIGAKPFLHDMPELPELD |
| | | NLFSPEGPILEAQKEAARLFGALET |
| | | WFLVGGSTCGVHAAIMATCSPGDTL |
| | | ILPRNSHISAISAMVLSGALPKYIV |
| | | PEYDFKWDVAGGITPSQVNDAIKEL |
| | | EMEGRKAAALLVVSPTYHELPSSSL |
| | | SQGADLAVQSTHKVLCSLTQSSMLH |
| | | MQGNLVNRERISKSLQMLQSSSPSY |
| | | LLLASLDAARAQLSENREAVFDKAM |
| | | DLALEARSLISKIPGISIIEFPSFS |
| | | SFFQIDPLRLTIGVWQLGLSGFEAD |
| | | DILCNDFGVVCELVGTKSFTLAFNL |
| | | GTQ |
| | | RDHVLRLVAGLNHLSQTSHFPQPVK |
| | | EEGKNVNHFVCLDDIRIRMSPREAF |
| | | FATKRKVSIRDSLGEICGELVCPYP |
| | | PGIPVLIPGEIITAEALNYLLEIRS |
| | | KGAVITGAADFSLSSFVVCVT* |
| SEQ | Solyc07g062700.3 | MRRSPNMFPKSFFTLLFIFILSTAV |
| ID | | YGGRVIVDQSSSDLISDGVHGGGGR |
| NO: | | NSTSIGRERGIISLFAAPVEEACEQ |
| 72 | | SYGFLPCTKTALGNVFLIVVYGYLM |
| | | FTAATYLSSGSELLLEILGPGLIGG |
| | | LFLPILGALPDAMLILVSGITGSAA |
| | | EAQSQVSVGIGLLAGSTVMLLTAIW |
| | | GTCCTVGKCDIVNSIAVDLKDTKGL |
| | | SFTGSGVSTDIWTSYAAMIMAVSVL |
| | | PFIVVQLPQIFHSNSGRHLSVLIAL |
| | | IISLSLLLSYCLYQVFQPWIQTRKL |
| | | DYVKHKHVISGILKHLKKHALGRLF |
| | | TDHGTPNIEVLEKLFNAIDENGDKH |
| | | LSHSELRALVVGIRLEEINLDENDA |
| | | IAKVMKDFDTSLDEQVDLHEFITGV |
| | | EKWLYEAKSTNGSSAQAGPNTINYI |
| | | DDFHEETRREHSLLGEDQSDEIVEG |
| | | VENPRKVAIKAASLLLLGTIIAAVF |
| | | ADPLVDAVNNFSSATSIPSFFISFI |
| | | ALPLATNSSEAVSAIIFATRKKQRS |
| | | ASLTFSELYGAVTMNNLLCLSVFLA |
| | | IVYARGLTWNFSSEVLVILIVCIVI |
| | | GVLGSIRTTFPLWTSLIAFAFYPFS |
| | | LVLVYVLDYVFGWS* |
| SEQ | Solyc07g064130.2 | MQIFVKTLTGKTITLEVESSDTIDN |
| ID | | VKAKIQDKEGIPPDQQRLIFAGKQL |
| NO: | | EDGRTLADYNIQKESTLHLVLRLRG |
| 73 | | GMQIFVKTLTGKTITLEVESSDTID |
| | | NVKAKIQDKEGIPPDQQRLIFAGKQ |
| | | LEDGRTLADYNIQKESTLHLVLRLR |
| | | GGMQIFVKTLTGKTITLEVESSDTI |
| | | DNVKAKIQDKEGIPPDQQRLIFAGK |
| | | QLEDGRTLADYNIQKESTLHLVLRL |
| | | RGGMQIFVKTLTGKTITLEVESSDT |
| | | IDNVKAKIQDKEGIPSDQQRLIFAG |
| | | KQLEDGRTLADYNIQKESTLHLVLR |
| | | LRGGMQIFVKTLTGKTITLEVESSD |
| | | TIDNVKAKIQDKEGIPPDQQRLIFA |
| | | GKQLEDGRTLADYNIQKESTLHLVL |
| | | RLRGGMQIFVKTLTGKTITLEVESS |
| | | DTIDNVKAKIQDKEGIPPDQQRLIF |
| | | AGKQLEDGRTLADYNIQKESTLHLV |
| | | LRLRGGF* |
| SEQ | Solyc07g065430.2 | MGWLRYVESGRKIDCISNPNTAYPI |
| ID | | PVLVEEVDDIISVAQYVIVVEKESV |
| NO: | | FQRLGRGYPDVPTRRFLRLLIDKLH |
| 74 | | LPVYGLVDCDPYGFDILTTYKFGSL |
| | | QMAYDATFLRVSEIQWLGVFVQDCD |
| | | NYSIPQQCLLPLTAEDKRKIEAMLP |
| | | RCYLQREVPQWRFELELLLHKGVKF |
| | | EIEALSVHSLTFLSHEYLPSKIHNG |
| | | GYIVDVHCDACKMKMVDVLSSICGV |
| | | YSVTIDSEDGTAKISGEVDPNLLLR |
| | | ALSRSGLGNHAEVKWVRLKHPMLSN |
| | | SHMTDGYGSYNHGYGSYNHGYGSHN |
| | | HGYGSYNPGYGYNSYGHGHGHGHGY |
| | | SSIGGPFMQRRSLPEYNYGYEGHHN |
| | | YQYPFRGTAQDSYATNYYSGALPSP |
| | | RPYNSYYM* |
| SEQ | Solyc07g065840.2 | MSDVETFAFQAEINQLLSLIINTFY |
| ID | | SNKEIFLRELISNSSDALDKIRFES |
| NO: | | LTDKSKLDGQPELFIHIIPDKANNT |
| 75 | | LTIIDSGIGMTKADLVNNLGTIARS |
| | | GTKEFMEALAAGADVSMIGQFGVGF |
| | | YSAYLVAEKVVVTTKHNDDEQYVWE |
| | | SQAGGSFTVTRDTSGENLGRGTKMV |
| | | LYLKEDQLEYLEERRLKDLIKKHSE |
| | | FISYPISLWVEKTIEKEISDDEEEE |
| | | EKKDEEGKVEEVDEEKEKEEKKKKK |
| | | VKEVSNEWSLVNKQKPIWMRKPEEI |
| | | TKEEYAAFYKSLTNDWEEHLAVKHF |
| | | SVEGQLEFKAVLFVPKRAPFDLFDT |
| | | KKKPNNIKLYVRRVFIMDNCDELIP |
| | | EYLSFVKGIVDSEDLPLNISRETLQ |
| | | QNKILKVIRKNLVKKCVELFFEIAE |
| | | NKEDYNKFYEAFSKNLKLGIHEDSQ |
| | | NRAKFAELLRYHSTKSGDEMTSLKD |
| | | YVTRMKEGQNDIYYITGESKKAVEN |
| | | SPFLEKLKKKGYEVLYMVDAIDEYS |
| | | IGQLKEFEGKKLVSATKEGLKLDES |
| | | EDEKKKQEELKEKFEGLCKVMKDVL |
| | | GDKVEKVIVSDRVVDSPCCLVTGEY |
| | | GWTANMERIMKAQALRDSSMAGYMS |
| | | SKKTMEINPENSIMDELRKRADADK |
| | | NDKSVKDLVLLLFETALLTSGFSLE |
| | | EPNTFGNRIHRMLKLGLSIDEESGD |
| | | ADADMPALEDPEADAEGSKMEEVD* |
| SEQ | Solyc08g006970.4 | PFELGDNSYKHNKYIIQTINFSIIS |
| ID | | SLFTMATPITKPPSPACTKIIEEIT |
| NO: | | RIYKSLPPRPSILEIEASISVVKSV |
| 76 | | ETEEKIELDEISKRRVEIEESVPAE |
| | | LYSVLEKVRKAMVLFQSKEQKKEAV |
| | | ELIELDKTYQDFDELIQEATELVCE |
| | | DTQMGKINSFEDPMVEIGKKDEVLK |
| | | KGLVTSGELINVSSSGIKHKEKYSL |
| | | MKVAALIENAAKTRARVVDLQNKLM |
| | | DKIEWLPLSLGKLVNVTELNVADNQ |
| | | IMALPTTIGSLNGLTKLDLHSNQII |
| | | NLPDSFGELINLTDLDLHANRLKSL |
| | | PASFRNLVNLIDLDLGSNRFTHLPD |
| | | FVGNLTSLKRLNVETNQLEELPYTI |
| | | GFCSSLVELRLDFNQLKALPEAMGM |
| | | LEHLEILTLHINRIKGLPTTMGNLS |
| | | RLRELDVSFNEVENIPETFCFAVSL |
| | | EKLNLANNFADLKTLPRSIGNLENL |
| | | EELDISNSQIRTLPDSFRLLSKLKT |
| | | FRADETPLEVPPRQIIKLGAQVVVE |
| | | YMAEFVTKNELQLQRPKRRPFFCPS |
| | | CLFPKTEGKRRI* |
| SEQ | Solyc08g029110.3 | MEKIRKASHAGSWYTDNPLELEEQL |
| ID | | DGWLRAAGLVKSSDVRGVIAPHAGY |
| NO: | | SYSGRAAAYAFGNIDPTNISRIFLL |
| 77 | | GPSHHYYTPKCALSRATVYKTPIGD |
| | | LPIDEEVNDELKATGHFEYMDLRVD |
| | | EAEHSMEMHLPYLAKVFQGYPVKIV |
| | | PILVGALSAESEALYGRFLAKYVDD |
| | | SKNFFSVSSDFCHWGSRFNYIHYDK |
| | | SHGAIYKSIEVLDKMGMDIIETGDP |
| | | DAFKLYLSETDNTICGRHPISVFLH |
| | | MLKNSSTKIKIRFLRYEQSSQCKSM |
| | | RDSSVSYASAVGKVDG* |
| SEQ | Solyc08g065220.3 | MERARKLANRAILKRLVSQSKQSRS |
| ID | | NEIPSPSSLYRPSRYVSSLSPYTFQ |
| NO: | | ARNSVKSFNTQQVRSISVEALKPSD |
| 78 | | TFPRRHNSATPEEQTKMAEFCGFQS |
| | | LDALIDATVPQSIRSESMKLPKFDG |
| | | GLTESQMIEHMQKLASKNKVFKSYI |
| | | GMGYYNTYVPPVILRNLLENPAWYT |
| | | QYTPYQAEISQGRLESLLNYQTMIT |
| | | DLTGLPMSNASLLDEGTAAAEAMAM |
| | | CNNILKGKKKTFLIANNCHPQTIEI |
| | | CKTRADGFDLKVVTVDLKDIDYKSG |
| | | DVCGVLVQYPGTEGEILDYGEFIKN |
| | | AHAHGVKVVMASDLLALTMLKPPGE |
| | | LGADIVVGSAQRFGVPMGYGGPHAA |
| | | FLATSQEYKRMMPGRIIGLSVDSTG |
| | | KPALRMAMQTREQHIRRDKATSNIC |
| | | TAQALLANMAAMYAVYHGPEGLKTI |
| | | GQRVHGLAGTFSAGLKKLGTVEVQD |
| | | LPFFDTVKVKCSDAKAIADVATKND |
| | | INVRIVDNNTITVSFDETTTLEDVD |
| | | DLFKVFALGKPVPFTAQSIAQEVEN |
| | | LIPSGLTRETPFLTHQIFNSYHTEH |
| | | ELLRYLHKLQSKDLSLCHSMIPLGS |
| | | CTMKLNATTEMMPVTWPSFANIHPF |
| | | APTEQAAGYQEMFDDLGALLCTITG |
| | | FDSFSLQPNAGAAGEYAGLMVIRAY |
| | | HMSRGDHHRNVCIIPVSAHGTNPAS |
| | | AAMCGMKIVAVGTDAKGNINIEELR |
| | | KAAEAHKDNLSALMVTYPSTHGVYE |
| | | EGIDEICKIIHDNGGQVYMDGANMN |
| | | AQVGLTSPGFIGADVCHLNLHKTFC |
| | | IPHGGGGPGMGPIGVKKHLAPYLPS |
| | | HPVVSTGGIPSPDQSKPLGAISAAP |
| | | WGSALILPISYTYIAMMGSKGLTDA |
| | | SKIAILSANYMAKRLEKHYPVLFRG |
| | | VNGTCAHEFIIDLRGFKNTAGIEPE |
| | | DVAKRLIDYGFHGPTMSWPVPGTLM |
| | | IEPTESESKAELDRFCDALISIREE |
| | | IAQIEKGNVDINNNVLKGAPHPPSM |
| | | LMADAWTKPYSREYAAYPAPWLRSA |
| | | KFWPTTGRVDNVYGDRNLICTLLPV |
| | | SEMAEEKAATA* |
| SEQ | Solyc08g069120.4 | MHVEPHRRALSVERWVCLGRAIGRL |
| ID | | GPHFLFQQNTKSSSFFFTALLFSAR |
| NO: | | VSGISRCVLKMSRVYVGNLDPRVSE |
| 79 | | RELEDEFRIFGVIRSVWVARRPLAT |
| | | LLLTSMIGGMHKMQSKSWMVRMDGE |
| | | WSFHIILEVEVVGAVEEVEVDLEAL |
| | | I* |
| SEQ | Solyc08g075860.3 | MDYGCLKKIATKSPTTTIFLPYLLR |
| ID | | SVLTTRFQLLVAGSFCNSSGDHRNS |
| NO: | | EVFTSFSGSKEMEFFFRTQTEETSD |
| 80 | | CSFNGQDIQRCPFLSNINKPTNFSF |
| | | FSALNFPSPVKGGKGPIFEDGPNFD |
| | | MAFKVFHGKDGVVPLSGRSQFFNDN |
| | | IEVESAPQFNPLAAKAATISLSSFG |
| | | AGGPFSFDSFSRKWNSQKKKPESSK |
| | | KKKPSSQDKSSKHEAMGNEWLESGN |
| | | CPIAKSYRAVSGVLPLVASAFQLPP |
| | | GMKLKCPPAVVAARAALARTAFVKT |
| | | VRPQPLSSKMLVIGALGMAANIPLG |
| | | IWREHTEKFSLSWFTAVHAAVPFIA |
| | | MLRKSVVMPKTAMALTIAASILGQV |
| | | IGSRAERLRMKAKAESVKLVAQTGS |
| | | DGVIAQSSTEGVIAGLNSIQVSGMP |
| | | GVHCGTQGMLKDQPSKESANTISPS |
| | | ASLCF* |
| SEQ | Solyc08g076730.4 | MKTVLFRTGSGSIPAVVPGSQRAVS |
| ID | | VSTRESIGKVFNGEKKKGCSSPMIS |
| NO: | | LYSEVNRRGIRRVSSDSDVIRSGFE |
| 81 | | GSSVTRKLSKIGSRSLSSIIPEEDC |
| | | GSEQNELEVLRSIGSLSLTEDRRSY |
| | | AGDWHRNSIPVDELGFSGGGIGSTK |
| | | NKKFGGNGDGRDEIDADSLTGGKSD |
| | | PRKIGAYYKEMLNSDPMNPLLLRNY |
| | | GKYLHEVERDFVKAEECYGRAILAS |
| | | PGDGEVLSLYGKLVWETQRDENRAK |
| | | SYFDQAVHASPNDCTVLGSYAHFMW |
| | | EAEGDQDEDEDEERETVAAGASMVA |
| | | AY* |
| SEQ | Solyc08g082620.3 | MSSITYSNLILKRRESRINGGSHLQ |
| ID | | YNGLRAIETVKLTANSASCKPKAVK |
| NO: | | CGRIRAMASPAVSAPKRETDPKKRV |
| 82 | | VITGMGLVSVFGSDIDNFYNKLLEG |
| | | QSGISLIDSFDASSYPVRFAGQIRD |
| | | FSSEGYIDGKNDRRLDNCWRYCLVA |
| | | GKRALDDANLGQQVLETMDKTRIGV |
| | | LVGSGMGGLNVFSDGVEALLQRGYK |
| | | KVSPFFIPYSITNMGSALLAIETGL |
| | | MGPNYSISTACASANHSFCAAANHI |
| | | RRGDADIMVTGGTEAGVTATGVGGF |
| | | IACRALSQRNDDYEKASRPWDTNRD |
| | | GFVVGEGSGILVMESLEHALKRGAS |
| | | IIAEYLGGAATCDAHHMTDPRPDGL |
| | | GVWSCITKGLLDARISPEEVNYINA |
| | | HATSTLAGDLAEVNAIKKVFKDTSE |
| | | IKMNGTKSMIGHGLGAAGGIEAIVT |
| | | IKAITTGWLHPTINQHELEPQVTID |
| | | TVPNVKKQHEVNVGISNSFGFGGHN |
| | | SVVVFAPYRP* |
| SEQ | Solyc09g008280.2 | METFLFTSESVNEGHPDKLCDQVSD |
| ID | | AILDACLEQDPESKVACETCTKTNM |
| NO: | | VMVFGEITTKATVDYEKIVRDTCRG |
| 83 | | IGFVSADVGLDADNCKVLVNIEQQS |
| | | PDIAQGVHGHLTKKPEEIGAGDQGH |
| | | MFGYATDETPELMPLTHVLATKLGA |
| | | KLTEVRKNKTCPWLRPDGKTQVTVE |
| | | YKNDNGAMVPIRVHTVLISTQHDET |
| | | VINDQIAQDLKEHVIKPVIPAKYLD |
| | | ENTIFHLNPSGRFVIGGPHGDAGLT |
| | | GRKIIIDTYGGWGAHGGGAFSGKDP |
| | | TKVDRSGAYIVRQAAKSVVASGLAR |
| | | RCIVQVSYAIGVAEPLSVFVDTYKT |
| | | GTIPDKDILVLIKENFDFRPGMMSI |
| | | NLDLLRGGNYRYQKTAAYGHFGRDD |
| | | PDFTWETVKVLKPKA* |
| SEQ | Solyc09g009020.3 | MATIKSIKARQIFDSRGNPTVEVDV |
| ID | | HISNGVFARAAVPSGASTGIYEALE |
| NO: | | LRDGGSDYLGKGVSKAVNNVNSIIG |
| 84 | | PALVGKDPTDQTGLDNFMVHQLDGT |
| | | QNEWGWCKEKLGANAILAVSLAVCK |
| | | AGAAVRNVPLYKHIADLAGNKKLVL |
| | | PVPAFNVINGGSHAGNKLAMQEFMI |
| | | LPVGAANFKEAMKMGCEVYHHLKAV |
| | | IKKKYGQDATNVGDEGGFAPNIQEN |
| | | KEGLELLKTAIEKAGYTGKVVIGMD |
| | | VAASEFYGKDKSYDLNFKEESNDGS |
| | | QKISGDQLKDLYKSFVSEYPIVSIE |
| | | DPFDQDDWETYAKLTAEIGEQVQIV |
| | | GDDLLVTNPKRVAKAIAEKTCNALL |
| | | LKVNQIGSVTESIEAVKMSKKAGWG |
| | | VMTSHRSGETEDTFIADLAVGLSTG |
| | | QIKTGAPCRSERLAKYNQLLRIEEE |
| | | LGSEAVYAGASFRKPVEPY* |
| SEQ | Solyc09g064330.2 | MDAQSKSDSFSSSNLRIIVQNNPSE |
| ID | | SQLSELGIKSWPKWACSPGKYQLKF |
| NO: | | DAEETCYLLRGKVKVYPKNTIEMSP |
| 85 | | VEFGAGDLVIIPKGLCCTWDVTLPV |
| | | DKHYKFHSS* |
| SEQ | Solyc09g074240.1 | MFPLLHLFIFLCLSFSYCCYGQIIN |
| ID | | SSSSSCPLDFTSLRKIIDQNSKRPT |
| NO: | | LNSTAQCQYIRQGLRLVQSEYLRRT |
| 86 | | NSFFPPMSSADSCWNSYQSLVNDYV |
| | | QNFDIRSSCGFQTAWISQGCMNITT |
| | | RFEFESKVSPAAITNIVSSCNQSLE |
| | | NNSPCATCTTSLSGLASFLPGPSVG |
| | | NLFDCAAYPSVYAAAFANQFGPTDK |
| | | GTAKCLFLLDVSVGSSGKGKKSVVV |
| | | IVVVVVCVVVFAVVVFGYWFMWRKR |
| | | KIGLAKKWNTKRLESNLSSRLDSIS |
| | | GSTTLIRYSFDEIKAATKNFSRLNI |
| | | VGTGGYGNVYKGVLPGGIEVALKRF |
| | | KNCSVAGDANFTHEVEVIASVRHVN |
| | | LVALRGYCTATTPFEGHQRIIVCDL |
| | | MKNGSLHDHLFGTRHEKLSWGIRQK |
| | | VAIGTARGLAYLHYGAQPGIIHRDI |
| | | KASNILLDESFEPKVADFGLAKFTP |
| | | EGMTHLSTRVAGTMGYVAPEYALYG |
| | | QLTERSDVYSFGVVLLELLSGKKAI |
| | | MEFKDGQPTLVTDWAWSLVREGRAL |
| | | DVLEDSIPHLGPPEVMEKYVLVAVL |
| | | CSHPELYARPTMDQVVNMLDAEIPV |
| | | PTIPERPISLIADLDDIERSVSSGG |
| | | NSGNLSTAAGYQPYIFERETPLADS |
| | | DSVRARTLDQRSR* |
| SEQ | Solyc09g075090.1 | MRNMLFASLYPARLHFLKTLIFKIS |
| ID | | SSSTLSALDLPSRSSRTLYVGNLPG |
| NO: | | DVREREVEDLFYKYGPIAHIELKIP |
| 87 | | PRPPGYAFVEFEEARDAEDAIRGRD |
| | | GYDFDGHRLRVELAHGGRGNSSSND |
| | | RYGGGGGGRGQRGGGVSRRSDYRVL |
| | | VTGLPHSASWQDLKDHMRRAGDVCF |
| | | SQVFRDGSGTTGIIDYTNYDDMKYA |
| | | IKKLDDSEFRNAFSRATIRVKEYDR |
| | | SRSRSRSRSRSYSRGKSVSRSRSRS |
| | | RSRSRSKSKSKSPKVKSSKRSRSRS |
| | | RSVSSQSRSGSKGRPVSRFWICAAV |
| | | CGFAY* |
| SEQ | Solyc09g092380.3 | MALLVEKTTSGREYKVKDMSQADFG |
| ID | | RLEIELAEVEMPGLMASRAEFGPSQ |
| NO: | | PFKGAKITGSLHMTIQTAVLIETLT |
| 88 | | ALGAEVRWCSCNIFSTQDHAAAAIA |
| | | RDSAAVFAWKGETLQEYWWCTERAL |
| | | DWGPGGGPDLIVDDGGDATLLIHEG |
| | | VKAEEEFAKNGTVPDPTSTDNVEFQ |
| | | LVLTIIKESLKTDPLRYTKMKERLV |
| | | GVSEETTTGVKRLYQMQANGSLLFP |
| | | AINVNDSVTKSKFDNLYGCRHSLPD |
| | | GLMRATDVMIAGKVALVAGYGDVGK |
| | | GCAAAMKQAGARVIVTEIDPICALQ |
| | | ATMEGLQVLPLEDVVSEVDIFVTTT |
| | | GNKDIIMVDHMRKMKNNAIVCNIGH |
| | | FDNEIDMHGLETFPGVKRITIKPQT |
| | | DRWVFPDTNSGIIVLAEGRLMNLGC |
| | | ATGHPSFVMSCSFTNQVIAQLELWN |
| | | ERSSGKYEKKVYVLPKHLDEKVAAL |
| | | HLGKLGAKLTKLTKDQADYISVPVE |
| | | GPYKPAHYRY* |
| SEQ | Solyc09g097960.3 | MTKEGKNVKRIKLGSEGLEVSVQGL |
| ID | | GCLGMSDFYGPPKPEPDMIELIHHA |
| NO: | | INSGITFFDTSNSYGPYTNEILLGK |
| 89 | | ALKGGMRERVELATKFGIHFADGKI |
| | | EVRGEPEYVRAACEASLKRLDVDCI |
| | | DLYYQHRIDTRVPIEVTVGELKKLV |
| | | EEGKIKYIGLSEASASTIRRAHAVH |
| | | PITAVQLEWSLWSRDVEEEIIPTCR |
| | | ELGIGIVAYSPLGRGFLSSGPKLVE |
| | | DLTNEDFRKHLPRFQGENLEHNKKL |
| | | YERICELATKKGCTPSQLALAWVHH |
| | | QGNDVCPIPGTTKIENFNQNIGALS |
| | | IKLTSEDMAELESIASANAVQGDRY |
| | | GSAAATYKDSETPPISAWKAT* |
| SEQ | Solyc09g098520.4 | MKNGMAECAVCHSKLVSPSVKTISR |
| ID | | AYDRHRSKISSKQRALNVLLVVGDC |
| NO: | | MLVGLQPVLVFMSKVDGKFKFSPVS |
| 90 | | VNFLTEATKVVFAIIMLLIQARHQK |
| | | VGEKPLLSISTFVQAARNNVLLAVP |
| | | ALLYAINNYLKFIMQLYFNPATVKM |
| | | LSNLKVLVIAVLLKFIMKRRFSVIQ |
| | | WEALALLLIGISINQLRSLPEGTTS |
| | | LALPVTTIAYIYTLIFVTVPSMASV |
| | | FNEYALKSQYDTSIYLQNLFLYGYG |
| | | AIFNFLAILGIAVFKGPGSLDIFQG |
| | | HSKATMLLIVNNAAQGILSSFFFKY |
| | | ADTILKKYSSTVATIFTGIASALLF |
| | | GHTLTVNFLLGISVVFISMHQFFSP |
| | | LSKVKDDQQNGTVELIDVRENHSPK |
| | | DSSFVNMAAGANEEASHRVGPDERQ |
| | | PLLPR* |
| SEQ | Solyc10g005510.3 | MAFSSLLKPAASFVRPSHRSQVSCA |
| ID | | GLHHSSNSVKLQSSIFGDAVTILQS |
| NO: | | SSLQKSGACSIQPIRATATELPPTV |
| 91 | | PRSQTGGKTRVGINGFGRIGRLVLR |
| | | IATFRDDIEVVAVNDPFIDAKYMAY |
| | | MLKYDSTHGVYSGSISVLDDSTLEI |
| | | NGKQIKVSSKRDPADIPWGDLGADY |
| | | VVESSGVFTTIDKASAHKKGGAKKV |
| | | VISAPSADAPMFVVGVNEKTYKANM |
| | | DVVSNASCTTNCLAPLAKVVHEEFG |
| | | IVEGLMTTVHATTATQKTVDGPSMK |
| | | DWRGGRGAGQNIIPSSTGAAKAVGK |
| | | VLPELNGKLTGMAFRVPTPNVSVVD |
| | | LTCRLDKSASYDDVKAAIKYASEGP |
| | | LKGILGYTDEDVVSNDFVGDSRSSI |
| | | FDAKAGIGLSKSFVKLVSWYDNEWG |
| | | YSNRVLDLIEHMALVASIH* |
| SEQ | Solyc10g008420.3 | MAGKQLDVPRTSGGNLKEQLVRRTL |
| ID | | QNVRSQGHIYVELREDGKRLIFFCT |
| NO: | | LCHSPCYSDSVLFNHLKGNLHTEML |
| 92 | | AAAKATLLKPNPWPFNDGVLFFNDP |
| | | EQDKQDKQSPNVNVGKSRLVDTCLE |
| | | DESSVAIVEYDDNLRHNEDTYVSEY |
| | | EYGLLDSELIGNEESDYLVIPGVLC |
| | | KDELSDLEVKHIGIGKIAARISVRG |
| | | IDSKSIRRIWCEWLAKKDSDDMDTS |
| | | VVPDHDFAVVTFPYNYNLGRSPLLD |
| | | DRFLLPSSPYSESEETSVTGKRKRK |
| | | SFSDPEDFSESLSNHCDSSGEESQS |
| | | TNNSNMKLILGTCDDQLVSSRIISS |
| | | KTMRRELRKQQRVASERMCDICQQK |
| | | MLPGKDVATLLSWKSGKLMCSSRNM |
| | | SGAFHLFHVSCLIHWILQCELQTSV |
| | | KPVDEPKMEPKAKRRSKKKTGTKHN |
| | | AKEKEDETKSARRINSVFCPECQGT |
| | | GICIEGDELEKPPVSLSEVYRLKIK |
| | | LSDARKAWMKNPEVLQNCSTGFDLP |
| | | PEHDDLLQEYVSPLKLLHFYRANVS |
| | | H* |
| SEQ | Solyc10g081120.3 | MESRHSIHHVLLLVLFGLSALCQCS |
| ID | | ATGVEANQTAVLLVNASEASARRIP |
| NO: | | DTLFGIFFEEINHAGAGGLWAELVN |
| 93 | | NRGFEGGGPNVPSNIDPWSIIGDES |
| | | KVIVSTDRSSCFDRNKIAVQVQVLC |
| | | DHTGANICPDGGVGIYNPGFWGMNI |
| | | EQGKSYKLVLYVRSEESVNVSVALT |
| | | GSNGLQKLAAANIVAADVSSWTKVE |
| | | ILLEATGTDPNSRLELRSSKKGVIW |
| | | FDQVSLMPTDTYKGHGFRKDLFGML |
| | | KDLKPAFIRFPGGCFVEGDWLRNAF |
| | | RWKETIGQWEERPGHFGDVWNYWTD |
| | | DGLGHFEFLQLAEDLDSLPVWVFNN |
| | | GVSHHDQVDTSSILPFVQEILDGLE |
| | | FARGDPTSTWGSIRAKMGHPEPFDL |
| | | RYVAIGNEDCGKTQYRGNYLKFYTA |
| | | IKDKYPDIKIISNCDGSTRPLDHPA |
| | | DLYDFHIYSSASSVFSNARHFDSAP |
| | | RRGPKAFVSEYAVTGNDAGKGSLLA |
| | | ALGEAGFLIGVEKNSEAIEMASYAP |
| | | LFVNDNDRRWNPDAIVFTSSQMYGT |
| | | PSYWMQHFFKESNGATLLSSSLQAN |
| | | PSNSLIASAITWRNSLDNNDYLRIK |
| | | VVNFGTTAVITKISLTGLGQNSLET |
| | | LFGAVMTELTSNNVMDENSFREPNK |
| | | VIPVKTQVEKVSDNMDVVLAPRSLN |
| | | SIDFLLRKSINNNVDTASVLKSSC* |
| SEQ | Solyc11g006660.3 | MADGQENDKNIEIWKMKKLIKALES |
| ID | | ARGNGTSMISLIIPPGDQISRITKM |
| NO: | | LAEEYGTASNIKSRVNRQSVLGAIT |
| 94 | | SAQQRLKLYNKVPPNGLVLYTGTIM |
| | | TEDGKEKKVTFDLTPFKPINASLYL |
| | | CDNKFHTGPLGELLESDEKFGFIVM |
| | | DGNGTLFGTLSGNTREILHKFTVDL |
| | | PKKHGRGGQSALRFARLRMEKRHNY |
| | | VRKTAELATQFFINPATSQPNVSGL |
| | | ILAGSADFKTELSQSDMFDPRLQAK |
| | | ILNVVDVSYGGENGFSQAIELSAEI |
| | | LSNVKFIQEKRLIGKFFEEISQDTG |
| | | KYVFGVDDTIKALEMGAVETLVVWE |
| | | NLDINRYVLKNSVTNEIVIKHLNKD |
| | | QEADQSNFKDSENSAELEVQDKMPL |
| | | LEWFANEYKTFGCSLEFVTNRSQEG |
| | | SQFCRGFGGIGGILRYQLDMRSFDE |
| | | PSDEGEYFEDSD* |
| SEQ | Solyc11g006990.2 | MAKNSFLLHILLFFVLILSLCDGIL |
| ID | | SQYELEELTWLDDKDDEISMFHSQH |
| NO: | | SSMRRCDFTSGKWVFDQSYPLYDST |
| 95 | | CPYLSTAVTCTKNGRPDSDYEKWRW |
| | | KPHGCEIPRFNALEFLGRMRKKRIM |
| | | LVGDSIMRNQWESLVCLVQSVIPMA |
| | | RKTVTYVGPTMAFHAMDFETTIEFC |
| | | WAPFLVELKKGPENKRILHLDMIEE |
| | | NAKYWRGADVLVFDSAHWWTHSDKY |
| | | SSWDLIMEGNSFYRNMNPMVAYEKG |
| | | LMTWAKWVDLNLDPRKTRVFFRSMS |
| | | PRHNRENGWKCFNQREPLEFFSHPH |
| | | VPEPLLVLKEVLRGMSFPVSFQDIT |
| | | TMTALRRDGHPSVYSKFVSQTGKQH |
| | | LGDYKSDCSHWCLPGVPDTWNEMLN |
| | | VML* |
| SEQ | Solyc11g011380.2 | MSLLSDLINLNLSGDTQKIIAEYIW |
| ID | | IGGSGMDMRSKARTLPGPVTSPAEL |
| NO: | | PKWNYDGSSTGQAPGEDSEVILYPQ |
| 96 | | AIFKDPFRRGNNILVMCDAYTPAGE |
| | | PIPTNKRHAAAKVFSHPDVAAEETW |
| | | YGIEQEYTLLQREVNWPLGWPIGGF |
| | | PGPQGPYYCGTGADKAFGRDIVDAH |
| | | YKACLYAGINISGINGEVMPGQWEF |
| | | QVGPSVGISAGDEVWVARYILERIA |
| | | EIAGVVVSFDPKPIPGDWNGAGAHT |
| | | NYSTKSMREDGGYEIILKAIEKLGL |
| | | KHKEHIAAYGEGNERRLTGKHETAN |
| | | INTFKWGVANRGASVRVGRDTEKAG |
| | | KGYFEDRRPASNMDPYVVTSMIAET |
| | | TIIG* |
| SEQ | Solyc11g012705.2 | MIQENIVILLSLLLIVPISWAINNP |
| ID | | LIELSSREELAKIAGYGEEKLSTVF |
| NO: | | LHGTLLCGHTPHPVPGASVAVYCGT |
| 97 | | SGPGRVRKSSWAKNVTNESGDFIID |
| | | VPSHLHANIHDPNLCHIQILHLPKD |
| | | YSLCHHHSTKRKTFTLTSSNIGVRV |
| | | YTTPTIYLTPKASSRV* |
| SEQ | Solyc11g040110.2 | MLAHCTKSSLPAADAHLPTLRLPNP |
| ID | | RRSPAPELHFGFNSFSTRTSKLQVK |
| NO: | | ALDRNSNNSSTGDGVGASKNAASSP |
| 98 | | SSSVPNTNYVVPLDKASSCITRPLA |
| | | EILRDLNKRIPDNIIKAGNNDDHST |
| | | FITWYHANRMLSFYAPGWCGEVRDV |
| | | IFSDNGSVTVVYRVTIRGSDGEAHR |
| | | ESTGTVSPGNGQYTDVVAAAEEIAF |
| | | CRACARFGLGLYLYH* |
| SEQ | Solyc11g066130.1 | MASSSAKILLPLSLLFTLLSLSQST |
| ID | | NPNFILTLVNNCPYTIWPAIQPNAG |
| NO: | | HPVLERGGFTLHSLTHRSFPAPNAH |
| 99 | | WSGRIWARTGCNYQHGKFYCATGDC |
| | | GGRIECDGLGGAAPATLAQFVLHHG |
| | | HADFSTYGVSLVDGFNIPLTVTPHE |
| | | GKGVCPVVGCRANLLESCPAVLQFR |
| | | SHGGHGPVVGCKSACEAFKSDEFCC |
| | | RNHYNSPQTCKPSSYSQFFKHACPA |
| | | TFTYAHDSPSLMHECSSPRELKVIF |
| | | CH* |
| SEQ | Solyc12g005140.2 | MIGWFKQLHTAFGFSFLWLVCLIYF |
| ID | | TQGFRSFVWTAVSYQLKDNLKLSPS |
| NO: | | ASQFVTSIAFFPWSIKPLYGILSDC |
| 100 | | FPIRGRKRVPYLMLATLLSLLPWLI |
| | | LGLSDFLRNTRTELMILLTLQNLGS |
| | | AMADVVIDAMIAEAVRFERASFAGD |
| | | LQSVSWLSMAFGGICGSLLGGYALD |
| | | NIRIDMIFLLFSVLPALQLFSCGLV |
| | | EEGSVQGKALPEGSALNGSDMTSGI |
| | | VDEENKFSGEKSKVSTLRRKKSQNN |
| | | TKRVPTTESKFQAPDKKGSSLSPWY |
| | | QSLKMATSTLFRAFRQPAILRPMAW |
| | | FFLAHVTVPNLSTVMFYYQTEFLKL |
| | | EASFLGTARVLGWLSLMIGTLTYNR |
| | | YLKRMRLRHILMFTQVCLAILTILD |
| | | MVLVSRANVSLGISDKVTVLFGSAL |
| | | SDGINQFKFMPFLILSGQLCPPGVE |
| | | GTLFALFMSINNLGSTVGSFVGAGL |
| | | ASVLNISSGSFDNLFLGIGIQVLCT |
| | | FIPVAFLFLIPKEATGISA* |
| SEQ | Solyc12g088720.2 | MAQIQVQHQTPVAGGNGVAVAAAAA |
| ID | | GAAAAVAAPGGGATSQFTSTSLYVG |
| NO: | | DLDFNITDSQLYDLFNHVGQVVSVR |
| 101 | | VCRDLSTRRSLGYGYVNYSNPSDAA |
| | | RAMELLNFTPVNGKSIRVMYSHRDP |
| | | TLRKSGSANIFIKNLDKSIDNKALH |
| | | DTFSSFGNILSCKIATDSNGQSKGY |
| | | GFVQFDNEESAQGAIDKLNGMLMND |
| | | KQVYVGHFLRKQERESTTGMTKFNN |
| | | VYVKNLAESATDDELKNVFGEFGTI |
| | | TSAVVMRDADGKSKGFGFVNFENAD |
| | | DAAKAVEALNGKKFDEKEWYVGKAQ |
| | | KKSEREQELKNKFEQTAKEAVDKYQ |
| | | GVNLYVKNLDDTIDDEKLKELFAEF |
| | | GTITSCKVMRDPSGISRGSGFVAFS |
| | | TPEEASRALSEMNGKMIVSKPLYVA |
| | | LAQRKEERRAKLQAQFSQLRPVAMP |
| | | PSLAPRMPIYPPGAPGIGQQLFYGQ |
| | | GPPALIPPQAGFGYQQQLVPGMRPG |
| | | GAPMPNFFMPMVQQGQQGQRPGGRR |
| | | GAGPVQQSQQPMPLMQQQMMPRGRM |
| | | YRYPPGRNVPDGPMPGVAGGMLSVP |
| | | YDMGGMLPRDAAAMGQPVPISTLAT |
| | | ALANAPPEQQRTMLGESLYPLVDQL |
| | | EHEHAAKVTGMLLEMDQTEVLHLLE |
| | | SPEALKAKVSEAMDVLRNVQQATSS |
| | | PADQLASLSLNDNLVS* |
| SEQ | Solyc12g095880.2 | MGTESNYQMKVVKGDHGYVLEDVPH |
| ID | | LTDYIPDLPTHDNPLRSNPAYSVVK |
| NO: | | QYFVDMDDTVPQKVVVHKDSPRGVH |
| 102 | | FRRAGPRQKVYFNSDDVRACIVTCG |
| | | GLCPGLNTVIREIVHSLDYMYGVSK |
| | | VFGIDGGYRGFYSKNIINLTPKTVN |
| | | DIHKRGGTILGSSRGGHDTTKIVDS |
| | | IQDRGINQVYIIGGDGTQKGAAVIY |
| | | EEIRRRDLKVIVAGIPKTIDNDIPV |
| | | IDKSFGFDTAVEEAQRAINAAHVEA |
| | | ESAENGIGVVKLMGRYSGFIAMYAT |
| | | LASRDVDLCLIPESPFYLEGDGGLF |
| | | EYIEKRLKENGHMVIVIAEGAGQEL |
| | | LAEENAHAKNEQDASGNKLLQDVGL |
| | | WISQKIRDHFAKKTKMPITLKYIDP |
| | | TYMIRAVPSNASDNVYCTLLAQSCV |
| | | HGAMAGYTGFTSGLVNGRQTYIPFN |
| | | RITEKQNMVVITDRMWARLLSSTNQ |
| | | PSFLRVKDIEEIKKEEQPQTQLLDG |
| | | DNNVHENSGH* |
| SEQ | Solyc12g099970.2 | MGNANAREDGAAGDGDGDGQVSGRR |
| ID | | SNVESGIVEDHHALNSRVPSADLMV |
| NO: | | NSPPQSPHRSASPLLFGPQVPVVPL |
| 103 | | QGGDGNPVSNQMWGNECEDASDHSL |
| | | EGGIPTLITWSYGGNNVAIQGSWDN |
| | | WTSRKILQRSGKDYTVLLVLPSGIY |
| | | HYKFIVDGEVRYIPELPCVADETGV |
| | | VFNLLDVNDNVPENLESVAEFEAPP |
| | | SPDSSYAQALMGEEDFEKEPVAVPP |
| | | QLHLTVLGSENSEEAPSSPKPQHVV |
| | | LNHLFIEKGWASQSVVALGLTHRFQ |
| | | SKYVTVVLYKPLKR* |
| SEQ | Solyc05g054040.4 | MCTKFVKEMEKMALYVVVLLIICYA |
| ID | | SPARSDGSDHKYKSGDQVPLYANKV |
| NO: | | GPFHNPSETYRFFDLPFCTPDHVKE |
| 104 | | KKEALGEVLNGDRLVSAPYKLDFLY |
| | | DKDSEIVCKKKLSKEEVSQFRSAVA |
| | | KDYYFQMYYDDLPIWGFLGKVDKEG |
| | | KSDPSEYKYYLFKHLHFEIFYNDDR |
| | | VIEINARTDPNALVDITEDKEVDVD |
| | | FMYTVKWKETKTPFDKRMEKYSQSS |
| | | SLPHHLEIHWFSIINSCVTVLLLTG |
| | | FLATILMRVLKNDFVKYAHDEEAAD |
| | | DQEETGWKYIHGDVFRYPSHKSLMA |
| | | AALGSGTQLFTLTIFIFLLSLVGVF |
| | | YPYNRGALFTALVVIYALTSGIAGY |
| | | TAASFYCQLEGTNWVRNLILTGALF |
| | | CGPLFLTFCFLNSVAIAYSATAALP |
| | | FGTIVVIFLIWALVTSPLLVLGGIA |
| | | GKNSRAEFQAPCRTTKYPREIPPLP |
| | | WYRGTLPQMAMAGFLPFSAIYIELY |
| | | YIFASVWGHRIYTIYSILFIVFIIL |
| | | IIVTAFITVALTYFQLAAEDHEWWW |
| | | RAFLCGGSTGLFIYGYCLYYYYARS |
| | | DMTGFMQTSFFFGYMACICYGFFLM |
| | | LGSVGFRAALFFVRHIYRSIKCE* |
| SEQ | Solyc11g011910.2 | MCTKFVKEMEKMALYVVVLLIICYA |
| ID | | SPARSDGSDHKYKSGDQVPLYANKV |
| NO: | | GPFHNPSETYRFFDLPFCTPDHVKE |
| 105 | | KKEALGEVLNGDRLVSAPYKLDFLY |
| | | DKDSEIVCKKKLSKEEVSQFRSAVA |
| | | KDYYFQMYYDDLPIWGFLGKVDKEG |
| | | KSDPSEYKYYLFKHLHFEIFYNDDR |
| | | VIEINARTDPNALVDITEDKEVDVD |
| | | FMYTVKWKETKTPFDKRMEKYSQSS |
| | | SLPHHLEIHWFSIINSCVTVLLLTG |
| | | FLATILMRVLKNDFVKYAHDEEAAD |
| | | DQEETGWKYIHGDVFRYPSHKSLMA |
| | | AALGSGTQLFTLTIFIFLLSLVGVF |
| | | YPYNRGALFTALVVIYALTSGIAGY |
| | | TAASFYCQLEGTNWVRNLILTGALF |
| | | CGPLFLTFCFLNSVAIAYSATAALP |
| | | FGTIVVIFLIWALVTSPLLVLGGIA |
| | | GKNSRAEFQAPCRTTKYPREIPPLP |
| | | WYRGTLPQMAMAGFLPFSAIYIELY |
| | | YIFASVWGHRIYTIYSILFIVFIIL |
| | | IIVTAFITVALTYFQLAAEDHEWWW |
| | | RAVIRLWWINRLVHLWLLPVLLLCP |
| | | IRHVRFHANLILLWLHGLHLLCLLS |
| | | HAWNCWFPSCFVFRPPHIPFNQVRV |
| | | X |
| SEQ | Solyc09g092390.2 | MALLVEKTTSGREYKVKDMSQADFG |
| ID | | RLEIELAEVEMPGLMASRAEFGPSQ |
| NO: | | PFKGAKITGSLHMTIQTAVLIETLT |
| 106 | | ALGAEVRWCSCNIFSTQDHAAAAIA |
| | | RDSAAVFAWKGETLQEYWWCTERAL |
| | | DWGPGGGPDLIVDDGGDATLLIHEG |
| | | VKAEEEFAKNGTIPDPSSTDNVEFQ |
| | | LVLTIIKESLKTDPLKYTKMKERLV |
| | | GVSEETTTGVKRLYQMQANGTLLFP |
| | | AINVNDSVTKSKFDNLYGCRHSLPD |
| | | GLMRATDVMIAGKVALVAGYGDVGK |
| | | GCAAAMKQAGARVIVTEIDPICALQ |
| | | ATMEGLQVLPLEDVVSEVDIFVTTT |
| | | GNKDIIMVDHMRKMKNNAIVCNIGH |
| | | FDNEIDMHGLETFPGVKRITIKPQT |
| | | DRWVFPDTNSGIIVLAEGRLMNLGC |
| | | ATGHPSFVMSCSFTNQVIAQLELWN |
| | | EKSSGKYEKKVYVLPKHLDEKVAAL |
| | | HLGKLGAKLTKLTKDQADYISVPVE |
| | | GPYKPAHYRY* |
| SEQ | Solyc01g096590.3 | MAAYAAMKPTKPGLEEPAEMIHKIR |
| ID | | ITLSSKNVKNLEKVCADLVRGAKDK |
| NO: | | RLRVKGPVRMPTKVLNITTRKSPCG |
| 107 | | EGTNTWDRFELRVHKRVIDLFSSPD |
| | | VVKQITSITIEPGVEVEVTIAES* |
| SEQ | Solyc05g014470.3 | MANGKIKIGINGFGRIGRLVARVAL |
| ID | | QRDDVELVAVNDPFISTDYMTYMFK |
| NO: | | YDSVHGQWKHHELKVKDEKTLLFGE |
| 108 | | KAVTVFGIRNPEDIPWGEAGADFVV |
| | | ESTGVFTDKDKAAAHLKGGAKKVVI |
| | | SAPSKDAPMFVVGVNENEYKPELDI |
| | | VSNASCTTNCLAPLAKVINDRFGIV |
| | | EGLMTTVHAMTATQKTVDGPSMKDW |
| | | RGGRAASFNIIPSSTGAAKAVGKVL |
| | | PQLNGKLTGMAFRVPTADVSVVDLT |
| | | VRLEKEASYEDIKAAIKEESEGKLK |
| | | GILGYTEDDVVSTDFVGDSRSSIFD |
| | | AKAGIALSKNFVKVVSWYDNEWGYS |
| | | SRVIDLICHMAKA* |
| SEQ | Solyc05g007950.4 | MASNSAFSLFLILLIITQCLSVLNA |
| ID | | AKDFDFFYFVQQWPGSYCDTKQSCC |
| NO: | | YPTTGKPAADFGIHGLWPNNNDGTY |
| 109 | | PSNCDPNSPYDQSQISDLISSMQQN |
| | | WPTLACPSGSGSTFWSHEWEKHGTC |
| | | AESVLTNQHAYFKKALDLKNQIDLL |
| | | SILQGADIHPDGESYDLVNIRNAIK |
| | | SAIGYTPWIQCNVDQSGNSQLYQVY |
| | | ICVDGSGSSLIECPIFPGGKCGTSI |
| | | EFPTF* |
| SEQ | Solyc02g087540.3 | MVDTLTELCSKLAGLMFILGTIQQL |
| ID | | VPHSFYKRIKTLWHRIENYFYPYVQ |
| NO: | | ITIDEYSNYKRNEVYSLIKVYLGTK |
| 110 | | STNDAKYLKAEMFKKSKFLAVSLDE |
| | | GEEVIDEFHGVKLKWNSYTETFPDN |
| | | SSGGRSQLPIEKKCYTLTFNKKNQE |
| | | MVTGQYLKHVMEEGKAIEFKNKKQK |
| | | IYSNEEGERYWYGKGMWRNINFEHP |
| | | ATFDTLAMDPTKKEEIINDLVAFSK |
| | | GKDYYSKVGKAWKRGYLLYGPPGTG |
| | | KSTMIAAIANYLNYDIYDLELTSVK |
| | | DNSELKKLLMETTSKSIIVIEDIDC |
| | | SIDLTGKRKKKNKKETTNEEEEEDS |
| | | DSTTEKKNDKNESSKLTLSGLLNFI |
| | | DGIWSACGQERIIIFTTNHVDKLDP |
| | | ALIRRGRMDMHIEMSYCKYEAFKVL |
| | | AKNYLGIETHPLFQEIQSLLEEVDV |
| | | SPCDVAENLMPKNASGNPEICLESL |
| | | VKVLIKAKRKASLNSHKNKKSSTKL |
| | | IITYGRFKKLFQ* |
| SEQ | Solyc08g083120.2 | MQIFVKTLTGKTITLEVESSDTIDN |
| ID | | VKAKIQDKEGIPPDQQRLIFAGKQL |
| NO: | | EDGRTLADYNIQKESTLHLVLRLRG |
| 111 | | GIIEPSLMALARKYNQEKMICRKCY |
| | | ARLHPRAVNCRKKKCGHSNQLRPKK |
| | | KIK* |
| SEQ | Solyc05g050200.4 | MPKNKGKGGKNRKRGKNEADDEKRE |
| ID | | LVFKEDGQEYAQVLRMLGNGRCEAM |
| NO: | | CIDGTKRLCHIRGKMHKKVWIAAGD |
| 112 | | IILVGLRDYQDDKADVILKYMPDEA |
| | | RLLKAYGELPENTRLNEGIANGLDE |
| | | EDEGAGDDYIEFEDEDIDKI* |
| SEQ | Solyc11g005670.2 | MQIFVKTLTGKTITLEVESSDTIDN |
| ID | | VKAKIQDKEGIPPDQQRLIFAGKQL |
| NO: | | EDGRTLADYNIQKESTLHLVLRLRG |
| 113 | | GMQIFVKTLTGKTITLEVESSDTID |
| | | NVKAKIQDKEGIPPDQQRLIFAGKQ |
| | | LEDGRTLADYNIQKESTLHLVLRLR |
| | | GGMQIFVKTLTGKTITLEVESSDTI |
| | | DNVKAKIQDKEGIPPDQQRLIFAGK |
| | | QLEDGRTLADYNIQKESTLHLVLRL |
| | | RGGMQIFVKTLTGKTITLEVESSDT |
| | | IDNVKAKIQDKEGIPPDQQRLIFAG |
| | | KQLEDGRTLADYNIQKESTLHLVLR |
| | | LRGGMQIFVKTLTGKTITLEVESSD |
| | | TIDNVKAKIQDKEGIPPDQQRLIFA |
| | | GKQLEDGRTLADYNIQKESTLHLVL |
| | | RLRGGMQIFVKTLTGKTITLEVESS |
| | | DTIDNVKAKIQDKEGIPPDQQRLIF |
| | | AGKQLEDGRTLADYNIQKESTLHLV |
| | | LRLRGGMQIFVKTLTGKTITLEVES |
| | | SDTIDNVKAKIQDKEGIPPDQQRLI |
| | | FAGKQLEDGRTLADYNIQKESTLHL |
| | | VLRLRGGDL* |
| SEQ | Solyc12g088210.3 | MQIFVKTLTGKTITLEVESSDTIDN |
| ID | | VKAKIQDKEGIPPDQQRLIFAGKQL |
| NO: | | EDGRTLADYNIQKESTLHLVLRLRG |
| 114 | | GMQIFVKTLTGKTITLEVESSDTID |
| | | NVKAKIQDKEGIPPDQQRLIFAGKQ |
| | | LEDGRTLADYNIQKESTLHLVLRLR |
| | | GGMQIFVKTLTGKTITLEVESSDTI |
| | | DNVKAKIQDKEGIPPDQQRLIFAGK |
| | | QLEDGRTLADYNIQKESTLHLVLRL |
| | | RGGMQIFVKTLTGKTITLEVESSDT |
| | | IDNVKAKIQDKEGIPPDQQRLIFAG |
| | | KQLEDGRTLADYNIQKESTLHLVLR |
| | | LRGGMQIFVKTLTGKTITLEVESSD |
| | | TIDNVKAKIQDKEGIPPDQQRLIFA |
| | | GKQLEDGRTLADYNIQKESTLHLVL |
| | | RLRGGMQIFVKTLTGKTITLEVESS |
| | | DTIDNVKAKIQDKEGIPPDQQRLIF |
| | | AGKQLEDGRTLADYNIQKESTLHLV |
| | | LRLRGGF* |
| SEQ | Solyc12g099000.3 | METFLFTSESVNEGHPDKLCDQVSD |
| ID | | AVLDACLAQDPESKVACETCTKTNL |
| NO: | | VMVFGEITTKANIDYEKIVRDTCRE |
| 115 | | IGFVSPDVGLDADNCRVLVNIEQQS |
| | | PDIAQGVHGHLTKRPEEIGAGDQGH |
| | | MFGYATDETPELMPLSHVLATKLGA |
| | | RLTEVRKNGTCSWLRPDGKTQVTVE |
| | | YHNDNGAMVPLRVHTVLISTQHDET |
| | | VTNDEIARDLKEHVIKPVIPEKYLD |
| | | ENTIFHLNPSGRFVIGGPHGDAGLT |
| | | GRKIIIDTYGGWGAHGGGAFSGKDP |
| | | TKVDRSGAYIVRQAAKSIVANGLAR |
| | | RCIVQVSYAIGVPEPLSVFVDTYGT |
| | | GKIPDKEILNIVKENFDFRPGMISI |
| | | NLDLLRGGNGRFLKTAAYGHFGRDD |
| | | PDFTWEVVKPLKWDKPEA* |
| SEQ | Solyc01g096000.2 | MNDPYRSKPSSGRTNFASCICATVF |
| ID | | LIFLIAGIVVAYFFLFKPKTPKIAV |
| NO: | | EAVRFPTFSVTNGTVNFTFFQYVSV |
| 116 | | TNPNRDEFTHYDSSLQLSYSGEPVG |
| | | LVFIPAGKIDGGRTQHMSAKFDVQS |
| | | YPLPAKLKADVSGGMIPTTAVNVFD |
| | | EGPTMEVETRMKLVGRVRVLKVFTH |
| | | RVDSGVKCGVMIQVSSGSVLGVRC* |
| SEQ | Solyc10g006480.2 | MQIFVKTLTGKTITLEVESSDTIDN |
| ID | | VKAKIQDKEGIPPDQQRLIFAGKQL |
| NO: | | EDGRTLADYNIQKESTLHLVLRLRG |
| 117 | | GMQIFVKTLTGKTITLEVESSDTID |
| | | NVKAKIQDKEGIPPDQQRLIFAGKQ |
| | | LEDGRTLADYNIQKESTLHLVLRLR |
| | | GGMQIFVKTLTGKTITLEVESSDTI |
| | | DNVKAKIQDKEGIPPDQQRLIFAGK |
| | | QLEDGRTLADYNIQKESTLHLVLRL |
| | | RGGMQIFVKTLTGKTITLEVESSDT |
| | | IDNVKAKIQDKEGIPPDQQRLIFAG |
| | | KQLEDGRTLADYNIQKESTLHLVLR |
| | | LRGGMQIFVKTLTGKTITLEVESSD |
| | | TIDNVKAKIQDKEGIPPDQQRLIFA |
| | | GKQLEDGRTLADYNIQKESTLHLVL |
| | | RLRGGMQIFVKTLTGKTITLEVESS |
| | | DTIDNVKAKIQDKEGIPPDQQRLIF |
| | | AGKQLEDGRTLADYNIQKESTLHLV |
| | | LRLRGGF* |
| SEQ | Solyc01g095995.2 | MNDPYRSKPSSGRTNFASCICATVF |
| ID | | LIFLIAGIVVAYFFLFKPKTPKIAV |
| NO: | | EAVRFPTFSVTNGTVNFTFFQYVSV |
| 118 | | TNPNRDEFTHYDSSLQLSYSGEPVG |
| | | LVFIPAGKIDGGRTQHMSAKFDVQS |
| | | YPLPAKLKADVSGGMIPTTAVNVFD |
| | | EGPTMEVETRMKLVGRVRVLKVFTH |
| | | RVDSGVKCGVMIQVSSGSVLGVRC* |

Accordingly, certain embodiments of the invention provide a method of producing an RKN resistant plant cell or a plant comprising expressing, underexpressing, silencing, or overexpressing in the plant one or more genes encoding a sequence selected from SEQ ID NOs: 1 to 118 or homologs thereof. In preferred embodiments, the plant cell or a plant is a tomato plant cell or tomato plant.

TABLE 2
SEQ ID NOs: 119 to 129 providing amino acid
sequences of RKN effector proteins.

| | Protein Name | Protein Sequence |
|---|---|---|
| SEQ | Minc 13292 | MRSSTLFVLAVVLLAVIVQLHADPK |
| ID | (Effector 1) | AGQKPAATKEKAGEGKPTAKVSSDQ |
| NO: | | NKGKPKGQASNKIAHAAKEGAQHVK |
| 119 | | DAVSKLSHGNKPEVIGDDKLKTEIK |
| | | TVLDGMIKHPEKIKQGNKAKLVKAV |
| | | TCQVCKKINQDKNPKPKDQKLSKNQ |
| | | KRELKKEKKEKTKKLNAQIKKLLNS |
| | | DAALKKEMDDLFNAVKKAHEKKAAA |
| | | LPKTNSGHLKKPAIAHPAVPKNAQA |
| | | KPGHINVKQAHGQHNTNQAAALPIS |
| | | KSGPLKKLAGLKADLHKIPGHLSGK |
| | | LHTTQHVSGTKSAETPPKHSPINFE |
| | | NLLRNVSSPLARKNVENKLPNSSNP |
| | | SVPGALPSNMFGGKRKTEMDSEEDD |
| | | WDVGDVDDFEEFSNRPIGNKGLETS |
| | | GGKLSFPLKLAEDDYDNFEDDSLGD |
| | | DSFDNDSLGDVDEDGDLPNSSLGKA |
| | | ESDENGKGASEEDKSDELDVDDFKE |
| | | NELNSNLLKTDKDKKLLSEGNGDLD |
| | | EDLKKPSNEEVRKATELLQGAFSGL |
| | | QKNIESSKVNSALPMNDSLVKQIGN |
| | | PNLVDPVGA* |
| SEQ | Minc 18861 | MKSFIFFLFIALTIYSTTFAIPIKD |
| ID | ((Effector 2) | NDRPEFSLKNEENENAGKELILQKS |
| NO: | | KKDSKNVVNRATCYDNNLATYINNG |
| 120 | | LYYYPQNMGQLSNYILDRIKSARYS |
| | | GYWFVHAAIITGQTQGLHWQSSATG |
| | | DIFSPTSRHGCYYHDSYTYVVILKY |
| | | * |
| SEQ | Minc00801 | MRFDIFLLVAFSLGVAVNCGVVKRN |
| ID | ((Effector 3) | QNSYGDEAGAGGAAGAAPAAPAEAA |
| NO: | | APSGGEAAPAAAPAPEAAPAAAPAP |
| 121 | | EQAAPAVAQPAPAAAASQASGYRKK |
| | | RSQNSYGDEAGHAAGAAPAEAAPAP |
| | | AESAAPSGGEAAPAAAPAPEAAPAP |
| | | EAAPAAAPAPEQAAPAVAQPAPAAA |
| | | ASQASGYRKKRSQNSYGDEAGAGGA |
| | | AGAAPAESAAPAGGEAAAPAATGCT |
| | | ENCAAGGESAAPAAAPAPEAAPAAA |
| | | PAPEQAAPAVAQPAPAAAPSQASGY |
| | | RKKRSQNSYGDEAGHAAGAAPAESA |
| | | APSGGEAAAPSGGEAAAPAPEAAPA |
| | | PAPAEAAPAAAPAPEQAAPAVAQPA |
| | | PAAAPSQASGY* |
| SEQ | Minc02097 | MKLFVLLIGVLAFTVPKQQIQQVEY |
| ID | (Effector 4) | KQFPQPYKFVPISNIVKCDRKTKEC |
| NO: | | SVKLENLDPTNNYSLYTAKNDKGRG |
| 122 | | DKVKLTKVADIDLDKCQLDKNVKPE |
| | | VNGEEICNQIVKGIDDNAKAETIEV |
| | | NSGEIEFGSELEGTEDYAIVEKAMN |
| | | EKKEHKNQQAVEHVHIPGPGEQPVE |
| | | HNQPTIEYPTNSKQVHPADKYQHKL |
| | | EERAKKFGLSDFKHGDLYEDYRQQK |
| | | TVQEDEKDKRYQKVLGTLGDHKHPS |
| | | LVDQYNEDKGKFNQRVKSDPTGNKV |
| | | EKAKNSDSNGLEQKLEKLALSDFKH |
| | | GDLYKDYQQQITVRDDEKDKRYQKV |
| | | LGTLGDHKHPSLVEQYNRDKGKFNQ |
| | | RVKSDPTSNWHEDLFGKDYQRAMSD |
| | | FDHLKAKQREKILGTLEDHKHPSLI |
| | | DQYNKGSLNQRAKSDPTGNNIGKAK |
| | | NSNFNGSEQKLEKLALSDFKHGDLL |
| | | GRKGGIKQRTINVLAGKKIEHEKGS |
| | | DFNANVEEMIGAENGKANQVNPKLT |
| | | GRKLAEFNHIPAVDRILGFKRGGHA |
| | | LEEPHKN* |
| SEQ | Minc18033 | MSPSSFTLTAVLLEAIVFLYNRQVA |
| ID | ((Effector 5) | AMLSMHPSCSGRSSTIENKLKMSGG |
| NO: | | GNGINKFTPGNVSFPVACQYHSKNL |
| 123 | | KATNKKEYKISEDLPMNQEKLTNSK |
| | | EDDLIHKVKKIDKGNGAAVPYKTNK |
| | | NNEIGDGAENGKAVKIREIIFTEEQ |
| | | KKMTSEEFEHYLYSVPYDKNKKNKI |
| | | GKNENGEKVDKPNKEGGDTMFYSKA |
| | | GIIAKKIKEYVPTNGEFKIQTGLVY |
| | | RNNSFNASQDDSKNLLNISHILMAL |
| | | NENERDSQENLRNAADLFVALHECY |
| | | QLFSAIPLVFEVEMVLKKLEEEGNK |
| | | DDPIKLLEYFRLPTIKYPLLDLIKI |
| | | ENSTVSPDELIENVTKTIHKADNFI |
| | | AKNIHAFFINDNETFFNEIISRLET |
| | | ADMVLASIKKILKMFNNFNEKIPEN |
| | | FSMLKRLKPIEMHDLFENSKLLQKL |
| | | HAAILPGDEMKF* |
| SEQ | Minc01696 | MKSKAAAILMALMALYAEVVFGKAL |
| ID | ((Effector 6) | PIREIYSDLPPECKTLNLETVESIP |
| NO: | | TVITVNGKQYNIKGQIQHGATSRVF |
| 124 | | HAFEQHDNSPKRCMALKVVDLEKIE |
| | | HKQRNQATNEAKILGELNQKKVERI |
| | | PKLFEIEQHPTNKTISMVMELAHHD |
| | | LFDEITPHMGLGKHEWVRNTFKEIL |
| | | QAINPLHKIGFVHGDLKPENMAYFR |
| | | APRVINDQHFPTKDDPLVLKVIDFG |
| | | GVMKKQELSKACGEIVTGTVMYMSP |
| | | EQVAWCAAHDEEEFLARRKQFPLST |
| | | KSDTWSAGVILYQMLHGKSPYYWIT |
| | | KKYEHLDEEERGEKADAELRQTIFD |
| | | GKKPIEFDEKYQDLPLLEIVKATVR |
| | | CLARNPDDRPEIEQLLHEMDKLGHR |
| | | VEQLEGKIEKLEEEAEDLREEKEER |
| | | EEEKERDEEEKKDEEEEDEKYEEER |
| | | EEEEHRLAEERMKKQAQENARLQQE |
| | | ARFRYEQEMARRRQAQQANLQFFWN |
| | | NFLGGMNNGYGFFPLRNQFQNNQVG |
| | | QNFYPNNNNNNFAPPRMPQLAMPPP |
| | | LNVFLPQTMSPLPPPKPVQTQQQAY |
| | | PQTQKFIFPGLIEHLKEIHAIRASE |
| | | K* |
| SEQ | Minc00344 | MKFLIAVFIGFVAFEFCLAEDVSTS |
| ID | (Effector 7) | GYDEVPIDDLLPVLPPFCSDVNLNK |
| NO: | | NEKVANCCNAIIRESKIVVYNTTVG |
| 125 | | PVPNECSLSKCGRVCNRYFSQRRPE |
| | | SNTTEEPGCECQKKQTTKKPKHSCQ |
| | | SEPEASTPPPRNPYQSGPEPSTQQP |
| | | GGQVASHKPPSGPAAPSSRPPRNRY |
| | | PTEPETKPPRNPYQSGPEPSTQQPG |
| | | GIRQRSQASTPPPRNPYQSGPEPST |
| | | QQPGGQVASHKPPSGPAAPSSRPPR |
| | | NRYPTEPETKPPRNPYQSGPEPSTQ |
| | | QPGGQVASHKPPSGPAAPSSRPPRN |
| | | RYPTEPETKPPRNPYQSGPEPSTQQ |
| | | PGGQVASHKPPSGPAAPSSKPPRNL |
| | | YQSGPEPSTKPPRNLYPSGPEEQPG |
| | | ATTESEIVRL* |
| SEQ | Minc00469 | MMKLNLIFCILVLIAIIFYVNEIEA |
| ID | ((Effector 8) | RAKYADPSDVQGCGTGCCVGVDTVT |
| NO: | | CDTGKQSCNCDLCTC* |
| 126 | | |
| SEQ | Minc 15401 | MYFKAFLLLLISLLSAFVVNNARTI |
| ID | ((Effector 9) | SNMDEVGDVSATMTEKKNNLGEKSN |
| NO: | | QVLPQGEEEMDDLTEEYDDSDDSEE |
| 127 | | DEDNDDEEDDMDEDNDANNYDYLNS |
| | | RIDHMDDIDSFETYEELPHMDNYGK |
| | | MNEAKQMMPVMHMNAGENEHYKQLP |
| | | SIATQPINEANPIMPPMPVVQPEKM |
| | | EQAPEAMKTEATPQASQPSSPKTAA |
| | | PKAKTSKPKTELKKPSAKKVQATKG |
| | | AAKVTKKDAKKPKDVKKSKDPKAKT |
| | | AKGKVPKAKAAKGKPAKGKPAKPGK |
| | | GKPLKGKTAKASKAGSKKQPAKKGG |
| | | KKSGSKKVTAKGGKKH* |
| SEQ | Minc 12639 | MRQQIIENNLLSLAFLLFFLISLIN |
| ID | (Effector 11) | TINSKPHPNGLPMVRRDGNGGDLDD |
| NO: | | LLTEFRAKGSRMRFGKRSSSSSPRF |
| 128 | | SSSSADVDFIEAPIYYIPDRFMWFQ |
| | | * |
| SEQ | Minc03328 | MLKAASMFYSMIPFLILLCSLFENC |
| ID | (Effector 12) | GAGVPHAAVSSSQGVNIPHKISASM |
| NO: | | TKLGNPRVTHVTFPVKQNSSKSGIS |
| 129 | | LTVSDPKQPVLRASAKHDNQDLFSN |
| | | DENNDDDELQYYIKKYEEKERDEVA |
| | | RINLEDVEEHNKYMEEQEKLYEQTK |
| | | GKEGLDKISKNLLDRKHASYSKISS |
| | | YELIKEFYGIDLEYKIPKTIEQRKN |
| | | LVTKLLEGFIKMINKRNKEKFDIMI |
| | | SALGNENHKEKKGEFDEFLQYLELF |
| | | APDTILVSFLQIPDEGRQLLFETFD |
| | | EILTDLKKSKNKKDEHGKIIKGSSQ |
| | | HPAVHLKKYHDKLNNSDLDADTKER |
| | | IDQFFTIVQNDFNQLPIYLTENFAS |
| | | LFRKIATLGLPGLKEALIRYLEVWN |
| | | HADPQDQQMMANAFPKLAFFYNDSK |
| | | SMKMNDWAYRSVKGNFFFKIPLLIL |
| | | LKMIFLNKPKLENDSALYKELREKL |
| | | TKDFEANKELIDEIGKFFKPAKPLV |
| | | GSVKILN* |

As used herein, the term “overexpressing a gene” or grammatical variations thereof refer to a condition in a genetically modified plant cell or a genetically modified plant wherein the gene encodes for a protein at a level higher than the parent plant cell or the plant without the genetic modification. Thus, a parent plant cell or a parent plant is genetically modified to produce a modified plant cell or modified plant that expresses a gene to produce a protein at a higher level compared to the parent plant cell or parent plant.

As used herein, the term “reducing the expression of a gene,” “underexpressing a gene,” or grammatical variations thereof refer to a condition in a genetically modified plant cell or a genetically modified plant wherein the gene encodes for a protein at a level lower than the parent plant cell or the plant without the genetic modification. Thus, a parent plant cell or a parent plant is genetically modified to produce a modified plant cell or modified plant that expresses a gene to produce a protein at a lower level compared to the parent plant cell or parent plant.

As used herein, the term “silencing of a gene” or grammatical variations thereof refer to a condition in a plant cell or a plant wherein the gene is expressed at a lower level than the parent plant cell or the plant without the genetic modification without genetically modifying said gene. Gene silencing can be accomplished by transcriptional and/or post-transcriptional methods, including, for example, introducing an inhibitory compound, such as, for example, an inhibitory oligonucleotide.

Typically, overexpressing or underexpressing a gene in a plant cell or a plant comprises introducing into the plant cell or a plant, a nucleic acid construct comprising the gene. The nucleic acid construct is designed to induce or reduce the expression of the protein encoded by the gene. Methods of producing and introducing various nucleic acid constructs comprising genes of interest into a plant cell or a plant to overexpress or underexpress the genes are well known to a person of ordinary skill in the art and such embodiments are within the purview of the invention. Certain such embodiments are identified below.

A gene is referred to as “operably linked” when it is placed into a functional relationship with another DNA segment (for example, a promoter that is operably linked to a nucleic acid sequence encoding any one of SEQ ID NOs: 1 to 118 or homologs thereof). Enhancers may be operably linked to another DNA segment but need not be contiguous with the coding sequences whose transcription they control. Linking is accomplished by ligation at convenient restriction sites or at adapters or linkers inserted in lieu thereof. The expression cassette can include one or more enhancers in addition to the promoter. By “enhancer” is intended a cis-acting sequence that increases the utilization of a promoter. Such enhancers can be native to a gene or from a heterologous gene. Further, it is recognized that some promoters can contain one or more native, enhancers or enhancer-like elements. An example of one such enhancer is the 35S enhancer, which can be a single enhancer, or duplicated. See for example, McPherson et al., U.S. Pat. No. 5,322,938, which is hereby incorporated by reference in its entirety.

The promoter for driving expression of the genes of interest may be selected based on a number of criteria including, but not limited to, what the desired use is for the operably linked polynucleotide, what location in the plant is expression of the gene of interest desired, and at what level is expression of gene of interest desired or whether it needs to be controlled in another spatial or temporal manner. In one aspect, a promoter that directs expression to particular tissue may be desirable. When referring to a promoter that directs expression to a particular tissue is meant to include promoters referred to as tissue specific or tissue preferred. Included within the scope of the invention are promoters that express highly in the plant tissue, express more in the plant tissue than in other plant tissue, express poorly in the plant tissue, express less in the plant tissue than in other plant tissue, or express exclusively in the plant tissue. For example, “seed-specific” promoters may be employed to drive expression. Specific-seed promoters include those promoters active during seed development, promoters active during seed germination, and/or that are expressed only in the seed. Seed-specific promoters, such as annexin, P34, beta-phaseolin, alpha subunit of beta-conglycinin, oleosin, zein, napin promoters have been identified in many plant species such as maize, wheat, rice and barley. See U.S. Pat. Nos. 7,157,629, 7, 129,089, and 7,109,392. Such seed-preferred promoters further include, but are not limited to, Cim1 (cytokinin-induced message); cZ19B1 (maize 19 kDa zein); and milps (myo-inositol-1-phosphate synthase); (see WO 00/11177, herein incorporated by reference). The 27 kDa gamma-zein promoter is a preferred endosperm-specific promoter. The maize globulin-1 and oleosin promoters are preferred embryo-specific promoters. For dicots, seed-specific promoters include, but are not limited to, bean beta phaseolin, napin, beta-conglycinin, soybean lectin, cruciferin, and the like. For monocots, seed-specific promoters include, but are not limited to, promoters of the 15 kDa beta-zein, 22 kDa alpha-zein, 27 kDa gamma-zein, waxy, shrunken 1, shrunken 2, globulin 1, an LtpI, an Ltp2, and oleosin genes. See also WO 00/12733, where seed-preferred promoters from end1 and end2 genes are disclosed; herein incorporated by reference. Each of these aforementioned references is hereby incorporated by reference in its entirety, particularly as it relates to the promoters disclosed within the reference.

The promoters useful in the present invention can also include constitutive, inducible or tissue-specific (preferred) promoters that are operably linked to a gene encoding a protein comprising of any one of SEQ ID NOs: 1 to 118 or homologs thereof and are heterologous to the nucleic acid sequences to which they are operably linked. In other words, the promoters are not those found operably linked to a gene encoding SEQ ID NOs: 1 to 118 or homologs thereof in their native context within a plant. Constitutive promoters, generally, are active in most or all tissues of a plant; inducible promoters, which generally are inactive or exhibit a low basal level of expression, and can be induced to a relatively high activity upon contact of cells with an appropriate inducing agent; tissue-specific (or tissue-preferred) promoters, which generally are expressed in only one or a few particular cell types (e.g., root cells); and developmental- or stage-specific promoters, which are active only during a defined period during the growth or development of a plant. Often promoters can be modified, if necessary, to vary the expression level. Certain embodiments comprise promoters exogenous to the species being manipulated.

Non-limiting examples of root-specific promoters (a subset of tissue-specific promoters) include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664). Each of these aforementioned references is hereby incorporated by reference in its entirety, particularly as it relates to the promoters disclosed within the reference.

Exemplary constitutive promoters include soybean ubiquitin promoters, for example, the promoters for soybean ubiquitin B (UBB)/ubiquitin C (UBC) gene (certain examples of soybean ubiquitin promoters that could be used in the present invention are described in United States patent application publication numbers 20140053296 and 20100186119), the 35S cauliflower mosaic virus (CaMV) promoter (Odell et al. (1985) Nature 313:810-812), the maize ubiquitin promoter (Christensen et al. (1989) Plant Mol. Biol. 12:619-632 and Christensen et al. (1992) Plant Mol. Biol. 18:675-689); the core promoter of the Rsyn7 promoter and other constitutive promoters disclosed in WO 99/43838 and U.S. Pat. No. 6,072,050; rice actin (McElroy et al. (1990) Plant Cell 2:163-171); pEMU (Last et al. (1991) Theor. Appl. Genet. 81:581-588); MAS (Velten et al. (1984) EMBO J. 3:2723-2730); ALS promoter (U.S. Pat. No. 5,659,026); rice actin promoter (U.S. Pat. No. 5,641,876; WO 00/70067), maize histone promoter (Brignon et al., Plant Mol Bio 22 (6): 1007-1015 (1993); Rasco-Gaunt et al., Plant Cell Rep. 21 (6): 569-576 (2003)) and the like. Other constitutive promoters include, for example, those described in U.S. Pat. Nos. 5,608,144 and 6,177,611, and PCT publication WO 03/102198. Each of these aforementioned references is hereby incorporated by reference in its entirety, particularly as it relates to the promoters disclosed within the reference.

An inducible promoter/regulatory element is one that is capable of directly or indirectly activating transcription of a gene encoding one or more of SEQ ID NOs: 1 to 118 in response to an inducer. The inducer can be a chemical agent such as a protein, metabolite, growth regulator, herbicide or phenolic compound; or a physiological stress, such as that imposed directly by heat, cold, salt, or toxic elements, or indirectly through the action of a pathogen or disease agent such as a virus; or other biological or physical agent or environmental condition. A plant cell containing an inducible promoter/regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression.

Any inducible promoter/regulatory element can be used in the instant invention (See Ward et al., Plant Mol. Biol. 22:361-366, 1993). Non-limiting examples of such promoters/regulatory elements include: a metallothionein regulatory element, a copper-inducible regulatory element, or a tetracycline-inducible regulatory element, the transcription from which can be affected in response to divalent metal ions, copper or tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994). Inducible promoters/regulatory elements also include an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991; U.S. Pat. No. 6,504,082); a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84 (19): 6624-6628 (1987)), inducible by anaerobic conditions; and the light-inducible promoter derived from the pea rbeS gene or pea psalb gene (Yamamoto et al. (1997) Plant J. 12 (2): 255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90 (20): 9586-9590; Orozco et al. (1993) Plant Mol. Bio. 23 (6): 1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible promoter/regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genet. 227:229-237, 1991). Stress inducible promoters include salt/water stress-inducible promoters such as P5C'S (Zang et al. (1997) Plant Sciences 129:81-89); cold-inducible promoters, such as, cor15a (Hajela et al. (1990) Plant Physiol. 93:1246-1252), cor15b (Wlihelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338), and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331-340). Certain promoters are inducible by wounding, including the Agrobacterium pmas promoter (Guevara-Garcia et al. (1993) Plant J. 4 (3): 495-505) and the Agrobacterium ORF13 promoter (Hansen et al., (1997) Mol. Gen. Genet. 254 (3): 337-343). Each of these aforementioned references is hereby incorporated by reference in its entirety, particularly as it relates to the promoters disclosed within the reference.

Overexpression or underexpression of a gene comprising a nucleic acid sequence encoding any one of SEQ ID NOs: 1 to 118 or homologs thereof can also be achieved by one or one or more mutations in the endogenous promoter of the gene, wherein the one or more mutations increase or decrease the expression of the gene. For a particular gene, a skilled artisan can identify one or more mutations that would increase or decrease the expression of the gene and such embodiments are within the purview of the invention. In certain preferred embodiments, the overexpression is of a gene encoding an inactive protein.

Certain preferred embodiments of the invention provide a method of producing a plant cell or a plant that is resistant to RKN, the method comprising overexpressing or underexpressing in the plant cell or the plant a gene comprising a nucleic acid sequence encoding a protein, particularly a protein selected from SEQ ID NOs: 1 to 118 or homologs thereof. The plant cell or the plant can be a tomato plant cell or tomato plant.

Further embodiments of the invention provide a method of producing an RKN resistant plant cell or a plant comprising inactivating, overexpressing, silencing, or underexpressing in the plant one or more genes comprising a nucleic acid sequence encoding any one of SEQ ID NOs: 1 to 118 or homologs thereof. In preferred embodiments, the plant cell or a plant is a tomato plant cell or tomato plant.

Typically, inactivating a gene in a plant cell or a plant comprises introducing into the gene one or more mutations that inhibit, significantly reduce, or abolish the expression of an active protein from the gene. Mutations in a gene that inhibit, significantly reduce or abolish the expression of a protein from the gene can be achieved either by deleting the entire coding region of the gene or a portion of the coding region of the gene, by introducing a frame shift mutation within the coding region of the gene, by introducing a missense mutation, by introducing a stop codon or any combination of the aforementioned gene mutations. Inactivating a gene can also be performed by using molecular markers or other traditional breeding methods to integrate activated or inhibited genes in any plant germplasm, particularly the tomato germplasm. Further, inactivating one or more genes can be performed by introducing and/or expressing the one or more genes under endogenous promoters and/or any exogenous promoters.

In preferred embodiments, inactivating a gene in a plant cell or a plant comprises introducing into the gene one or more mutations that alters at least one, two, three, four, five, or more amino acid residues of the encoded protein. The introduced changes can either increase the expression, decrease the expression, or do not alter the expression of the gene An alternation of the amino acid sequence can comprise the deletion of amino acid, addition of an amino acid, or a change of an amino acid residue. Mutations of the nucleotide sequence can be achieved either by deleting the entire coding region of the gene or a portion of the coding region of the gene, by introducing a frame shift mutation within the coding region of the gene, by introducing a missense mutation, insertion of sequences that disrupt the activity of the protein encoded by the gene, by introducing a stop codon or any combination of the aforementioned gene mutations. Inactivating a gene can also be performed by using molecular markers or other traditional breeding methods to integrate activated or inhibited genes in any plant germplasm, particularly the tomato germplasm. Further, inactivating one or more genes can be performed by introducing and/or expressing the one or more genes under endogenous promoters and/or any exogenous promoters. The result of the alteration of at least one, two, three, four, five, or more amino acid resides can inactivate the protein.

Methods of inactivating a gene of interest in a plant cell or a plant to inhibit, significantly reduce, or abolish the expression of an active protein are well known to a person of ordinary skill in the art and such embodiments are within the purview of the invention. Certain such embodiments are identified below.

Sanagala et al. (2017), Journal of Genetic Engineering and Biotechnology; 15 (2): 317-321, describe several methods of inactivating a gene, for example, by implementing homologous recombination, zinc finger nucleases, Transcription activator-like effector nucleases (TALENs), and the clustered regularly interspaced short palindromic repeats/CRISPR-associated (CRISPR/Cas) system. The Sanagala et al. reference is incorporated herein by reference in its entirety.

In preferred embodiments, inactivating a gene, increasing the expression of a gene, or decreasing the expression of a gene of interest is performed using the CRISPR/Cas system. An example of such system to inactivate genes in a plant cell or a plant is provided by Ordon et al. (2017), The Plant Journal; 89:155-168. The Ordon et al. reference is incorporated herein by reference in its entirety.

Typically, a CRISPR/Cas system mediated inactivation of a gene involves the use of a guide RNA targeted to a gene of interest. A DNA oligomer targeted to a gene of interest can be transcribed into single guide RNA (sgRNA). sgRNA guides the Cas9 DNA endonuclease to the gene of interest by sgRNA hybridization to the target site. The endonuclease Cas9 makes a double strand break 3 bp upstream of Palindromic Adjacent Motif (PAM). The DNA breakage engages the repair mechanism, such as homologous recombination (HR) or the non-homologous end joining (NHEJ) mechanism. The NHEJ mechanism is a major double strand break repair pathway in plants and is known to be error prone. NHEJ DNA repair process introduces errors in the DNA repair, which causes irreversible mutations at the gene of interest. The chances of errors in DNA repair can be increased by providing multiple sgRNA. Based on the sequence of a gene comprising a nucleic acid sequence encoding any one of SEQ ID NOs: 1 to 118 or homologs thereof, a person of ordinary skill in the art can design and perform an inactivation of the gene using the CRISPR/Cas system and such embodiments are within the purview of the invention.

Methods of inactivating or silencing a gene of interest in a plant cell or a plant to inhibit, significantly reduce, or abolish the expression of an active protein also include introduction into the plant cell or the plant one or more inhibitory oligonucleotides, such as small interfering RNA (siRNA) or short hairpin RNAs (shRNA). Methods of producing and introducing inhibitory RNA are also well known to a person of ordinary skill in the art and such embodiments are within the purview of the invention.

Certain preferred embodiments of the invention provide a method of producing a plant cell or a plant that is resistant to RKN, the method comprising inactivating in the plant cell or the plant a gene encoding a protein.

Additional embodiments of the invention also provide a plant or a plant cell comprising an inactivated gene comprising a nucleic acid sequence encoding SEQ ID NOs: 1 to 118 or homologs thereof.

In the methods of producing a plant cell described herein the plant cell can be in a plant part, for example, a root. The plant can be a tomato plant.

Further embodiments of the invention provide methods for identifying a gene that induces RKN resistance when overexpressed, underexpressed, inactivated, silenced or overexpressed and inactivated in a plant cell or a plant, the method comprising the steps of: (a) overexpressing a gene in a plant cell or a plant, compared to the expression in a plant cell or a plant known to interact with RKN effector proteins, silencing a gene in a plant cell or a plant known to interact with RKN effector proteins, or inactivating or decreasing the expression a gene in a plant cell or a plant known to interact with RKN effector proteins, (b) testing the RKN resistance in the plant cell or the plant comprising the overexpressed, underexpressed, inactivated, silenced, or the overexpressed and inactivated gene, and (c) identifying the gene that induces resistance in the plant cell or the plant when overexpressed, underexpressed, inactivated, silenced or both overexpressed and inactivated in the plant cell or the plant when overexpressed or inactivated. In preferred embodiments, an overexpressed, underexpressed, silenced, and/or inactivated gene comprises a nucleic acid sequence of SEQ ID NOs: 130-247 or encoding any one of SEQ ID NOs: 1-118 or homologs thereof. In some embodiments, one or more genes comprising nucleic acid sequences of SEQ ID NOs: 130-247 or encoding SEQ ID NOs: 1-118 or homologs thereof are overexpressed or one or more genes comprising nucleic acid sequences of SEQ ID NOs: 130-247 (Table 3) or encoding SEQ ID NOs: 1-118 or homologs thereof are underexpressed, silenced, or inactivated. In certain embodiments, one or more genes comprising nucleic acid sequences of SEQ ID NOs: 130-247 or encoding SEQ ID NOs: 1-118 or homologs thereof are overexpressed and one or more genes comprising nucleic acid sequences of SEQ ID NOs: 130-247 or encoding SEQ ID NOs: 1-118 or homologs thereof are inactivated, silenced, or underexpressed. The plant cell or the plant can be a tomato plant cell or a tomato plant.

TABLE 3
SEQ ID NOs: 130 to 247 providing nucleotide sequences that encode proteins that can
confer RKN resistance when overexpressed, inactivated, silenced, or overexpressed
and inactivated.

| | Protein Name | Gene Sequence |
|---|---|---|
| SEQ | Solyc01g008960.3 | ATGGCTGAAGAAGAAACAAATGGAGCAGCAGCAGAGGGTC |
| ID | | TGCCACCTCCTCCCCCTGTTCCACCAGATTTCACTCCTGC |
| NO: | | TAAAACTGAAATTGAGCCTGTGAAGAAAAAGATCTTGCGC |
| 130 | | GTTCCCATGGCCAGGCGTGGTGTTGGAAATAAGGGACAAA |
| | | AGATTCAAATACTTACTAATCACTTTAAAGTGAATGTAAA |
| | | CAATGTGGATGGACACTTCTTCCACTACAGTGTTGCCTTA |
| | | TTCTATGAAGATGGTCGACCAGTTGATGGGAAGGGAGTTG |
| | | GAAGGAAGGTGTTAGACACAGTGCACGAAACTTATGATAC |
| | | AGAATTAGCTGGAAAGGACTTTGCCTATGATGGTGAAAAA |
| | | AGCTTGTTTACCATTGGAGCACTCCCTAGGAATAAAATGG |
| | | AGTTCACTGTTGTCCTTGATGACGTGATATCAAATAGGAA |
| | | CAATGGTAACAGCAGCCCCGGTGGGCATGGTAGTCCTAAT |
| | | GAAGCTGACAGGAAGAGGTTACGTCGTCCTTACCAGTCAA |
| | | AAACTTTCAAGGTGGAGATCAGTTTTGCTGCCAAAATCCC |
| | | GATGCAGGCGATTGCAAATGCTCTGCGTGGTCAAGAGTCA |
| | | GAGAACTCTCAAGAAGCCTTGCGAGTCTTGGATATTATTT |
| | | TGAGGCAGCATGCTGCAAAGCAGGGTTGCCTGCTTGTTCG |
| | | ACAATCCTTTTTCCACAATGATCCCAAGAACTTTGTTGAT |
| | | GTCGGCGCTGGTGTGCTTGGTTGTCGAGGATTCCATTCCA |
| | | GCTTCCGGACTACCCAGAGTGGCTTGTCTTTGAACATTGA |
| | | TGTGTCTACAACAATGATTATCCAGCCTGGGCCTGTCGTG |
| | | GACTTCTTGATTGCAAATCAAAATGCAAAGGATCCCTTTT |
| | | CACTGGATTGGGCAAAGGCAAAACGTGTGCTCAAGAATTT |
| | | GAGGGTGAAGACAACCCCCACTAATCAAGAGTACAAGATT |
| | | ACTGGACTGAGTGACCGTCCTTGTCGTGAGCAACTATTTA |
| | | CCCTGAAGCAGAAAGGAAAAGATGCAGATGGTGAGGTCCA |
| | | AACTACTGAAGTTACTGTGTTCGATTACTTTGTCAACCAC |
| | | CGCAACATAGAGTTGCGTTATTCTGCTGATTTACCCTGCA |
| | | TAAATGTTGGGAAACCAAAGCGTCCCACCTTTTTCCCAAT |
| | | TGAGCTATGTTCCTTGGTGTCTTTGCAAAGATACACAAAA |
| | | TCCTTGTCCACATTCCAGAGGTCTTCACTAGTTGAGAAAT |
| | | CAAGGCAAAAGCCTCAGGAGAGGATGCAAGTCTTAAGCAA |
| | | TGCTCTCAAAATCAACCAATATGATGCTGAGCCTCTGCTT |
| | | CGTTCCTGTGGAATATCCATTAGCAATAACTTCACTCAGA |
| | | TTGAAGGACGTGTTCTTCCTCCCCCAAAGCTGAAGACGGG |
| | | CGGTGATGACTTTGTCCCTCGTAATGGCAGATGGAATTTC |
| | | AATAATAAGAGACTAGTTGATCCCACTAAGATAGAACGCT |
| | | GGGCCGTTGTCAATTTTTCTGCACGCTGCAATGTACAAGG |
| | | GCTAGTCAGTGATCTGATCAAATGTGGAAAACAGAAAGGA |
| | | ATTATGGTGGAAGACCCATTTGATGTCTTTGAGGAGTCTC |
| | | CACAAGTCAGGAGGGCTCCACCACTTGTGAGGGTTGAGAA |
| | | GATGTTTGAGCAGGTTCAATCCAAGCTACCCGGGGCACCT |
| | | AAATTCCTGCTTTGTTTGCTTCCTGAGAGGAAAAACTGTG |
| | | ATGTATACGGACCATGGAAGCGAAAGAATCTTGCTGAGTA |
| | | TGGCATTGTTACCCAGTGTATAGCTCCAACAAGAGTCAAT |
| | | GATCAATATATCACCAATGTGCTCCTGAAGATAAACGCGA |
| | | AGCTTGGAGGTTTGAACTCTATGTTAACTGTTGAACATTC |
| | | TCCTGCAATCCCTATGGTGTCTAAGGTCCCTACCATTATT |
| | | CTTGGGATGGATGTGTCCCATGGCTCTCCTGGCCAATCTG |
| | | ATGTCCCTTCAATTGCAGCGGTTGTCAGTTCAAGGCAGTG |
| | | GCCATCAATATCTCGTTACAGAGCTTCAGTTCGTACTCAG |
| | | TCTCCTAAAGTGGAGATGATAGACAACTTGTTTAAACGTA |
| | | CCTCTGATACTGAAGATGATGGAATAATGAGGGAGGCGTT |
| | | GCTAGATTTTTATGTGAGCTCTGGAAAAAGGAAGCCCGAG |
| | | CATATAATAATATTCAGGGATGGTGTCAGTGAATCTCAGT |
| | | TTAGTCAAGTTCTGAATGTTGAGCTGGACCAGATTATTGA |
| | | GGCTTGTAAGTTTCTTGATGAGAAGTGGTCACCCAAGTTT |
| | | GTGGTGATAGTTGCCCAGAAGAACCATCATACCAAGTTTT |
| | | TCCAGCCTAATGATCCTAACAATGTTCCTCCAGGCACAAT |
| | | CATAGACAACAAAGTCTGTCACCCAAGGAATTATGACTTT |
| | | TACCTGTGTGCTCATGCTGGAATGATTGGTACAACTCGTC |
| | | CTACTCACTATCACGTCTTGTATGATGAGCTGGGTTTCTC |
| | | TGCTGATGATCTTCAAGAGCTTGTTCATAATCTCTCTTAT |
| | | GTGTACCAGAGAAGCACCACTGCAATATCTGTTGTTGCTC |
| | | CTATCTGTTATGCCCATCTTGCTGCTACTCAAATGGGACA |
| | | GTGGATGAAGTTTGAAGATGCATCAGAGACTTCCTCAAGT |
| | | CATAATGGAGTAACCAATGCTGGACCAGTCTCCGTTCCTC |
| | | AGCTTCCAAAGCTCGAGGAGAAAGTTTCCAGTTCTATGTT |
| | | CTTTTGCTAA |
| SEQ | Solyc01g014840.3 | ATGTCTTCTATTTGCCCACAAAATTGGAAGAATGATGTTT |
| ID | | TCTTGAGTTTTAGAGGTAAAGATACGCGTAAAACTTTTGT |
| NO: | | TGGTCATCTCAACTATGCTCTAAAACAAAAAGGAATTCAT |
| 131 | | ACATTCAAAGATGATGAAAGATTGGAAAGAGGAAAAACCA |
| | | TTTCACCTGAACTTGTTAAAGCTATTGAAGAATCAAGATT |
| | | TGCTATTGTTGTATTCTCTAAGAATTATGCATCATCCACT |
| | | TGGTGTTTAGATGAACTTGTGAAGATCATGGAATGTAAAA |
| | | AAGAATTAGGACAAACTGTGATACCTATATTCTATGATGT |
| | | AGATCCATCACATGTAACTAAACAAAGTGAAACTTTTGCT |
| | | AAATCCTTTGCTATACATGAGGAAAATTTGAAAGATGATG |
| | | TTGAGAAGGTGTTATGCTGGAGGGATGCATTTCGTCAAGC |
| | | AGGCAAAATAGCTGGATATGATTTGCCAAATGGCTATGAC |
| | | GGGTATGAATCTAATTGCATTCAGCATGTAGTTGAAGGCA |
| | | TACTGAGTAAATTATGTCAAGTTATTTCAACCATAGAAAA |
| | | TGATTTGGTGGGGATGGAATCTCGAGTGCAGGAAGTAAGT |
| | | TCATTACTAAGGATGGAAACACTCGATGTTCGTTTTATTG |
| | | GAATTTGGGGGATGGGAGGCATTGGTAAGACAACAATTGC |
| | | AAGAGCTGTGTTTGGCAAATATTGGGGCCAATTTGAAGGA |
| | | GTTTGTTTTCTGGATAATGTTGTAGAAATGCAAAAGACAC |
| | | ATGGACTGCAATATCTGCAAGGTGTTCTCCTCTCGAAAAT |
| | | CCTCAAAGTAAGTATAACTATAGCAAGTGTGTATGAAGGC |
| | | ATGAGAATCATAAAGGAAAGACTACGCTCGATGAAAGTTT |
| | | TGATCATTCTTGATGATGTAATTCAAAAGGACCAATTAGA |
| | | AATGTTAGTTGGAGGGCGTGATTGGTTTGGTATCGGCAGT |
| | | AGAATTTTGATTACTACAACAGATAAACATTTGTTACATA |
| | | ATCATGTGGTGGATGAAGTGTATTCAGTCAACTTGATGAC |
| | | TCGCGATGAAGCGATAGAGCTATTCAGCCTACACGCCTTT |
| | | AAGCAAAGAATTCCAGATAAAGATTTTGAGGAGCTATTAA |
| | | ATCAAGTTGTACAGACTGCCGGTTTTCTCCCTTTAGCTCT |
| | | GAAAATTCTAGGTTCGTCTCTCTGCGGGCTAGACAAGATT |
| | | GTGTGGAAATGCACAGTGGACAGACTTAAGGATATGCCAG |
| | | ATGATGTTCTTGCTAAGCTTAAGATAAGTTTTGATGGGCT |
| | | GAGTCCTTCTGATCAGAGACTATTTCTTGATATCGGATGC |
| | | TTTTATAGAGGAAAATTGAGGAGTTCTGTAGGGGAAATGC |
| | | TTGAGAGTTGTGATATTGGATCTACAATAAGAGTCTTGAT |
| | | TGAAAAGTCTCTCTTATTTGTCTCACCATATGACACAATC |
| | | GAAATGCATGATTTGATACAAGAAATGGCCTGGCACATCG |
| | | TGAGACAAGGCGACACAAGAAGGAGCAGAATATGGCTTCC |
| | | CGAGGACATCGAGGATTTGTTTACTGGAAATTTGGAGGCA |
| | | GAAGCTGTGGAGGGCCTATCGATCCCAAGGGATTACGTAC |
| | | GAAAACGGGATATATCATGTTACAACATAAATGAAACATT |
| | | CAGGAAAATGAAAAGATTAAGGGTGCTTGAAGTAAGAGCA |
| | | ACAAATTTCTGCTCTATTGATCCGATCACTCATCTTCCTA |
| | | GCAGCATAAGGTGGCTTGATTGGGAAGGTTGCCCTTTAAA |
| | | TTCATTGCCACAGAGTTTTGAACCTTCAAAGCTTCTTCGT |
| | | CTCGATATACTTGAAAGTACTACACTCCAGAAGCTCTGGT |
| | | CAATTTCAAAGGGATTGGACAAACTTAAGGCTTTGTACCT |
| | | CAGCTACTGTGAGAAGTTGGAAGAAGTTCCAAGCTTCGAT |
| | | ATAATGCCAAATTTAGAGAGAGTAAAGCTAGAGGGATGTA |
| | | ACAGTTTGAGAGAAGTCAGCCCATCTTTTGGAGTTCTCAT |
| | | GAAACTCACTTCAATGGAGCTAATTGATTGTCCGAGCCTC |
| | | AAGAAAATTCCAAGTTGTGTTGAGATGGGATCCCTTAAGA |
| | | GTCTCAAACTTTCTTGTCTTCCAAAATTGAGGGAATTGCC |
| | | AGAAACCAAGGGGATGCACCGTTTATTGACGTTAGAGCTG |
| | | ACTAACTGTCAATACCTTGAGAAGCTTCCAAATTTTGATC |
| | | AGATGGAATCCCTTGAGACTCTCAAACTTTCTTGTCTTCC |
| | | AAAAGTCACTATATTTCCGCCACCAGACGGAATGCACTGT |
| | | TTACAGGACATTGTTATCGAATATGTCCCGATAGTAGAGC |
| | | TTCCAGCATCAATCGGGAATCTTCGTTCCCTCAAACAACT |
| | | TAGATTAAGTCATTGTAAAGATCTAGTAAGCATTCCAAAC |
| | | AGCTTTTGTTGTCTGAAGAATCTAAGAGCCCTTCTTATCT |
| | | ACAATTGCAAAAGACTAATCTATTTGCCAGAAAAGATTGG |
| | | TGACTTGAAACTGTTAAAGAAGCTAGTAGTATCTGGTACT |
| | | TCAATTTCCCAGATACCAACTTCAGTTACAGATCTTCGTG |
| | | AGCTAAGCTTCTTATCGTTTTCTCGTTGGTCTGGATACAG |
| | | ACAAGGTGCAACTTTTCTGTTCCCGCCTTCATCAGGTGTA |
| | | TCGTTGCCGTTGAGGGTGTTAAAGCTAAAAAAAAACAGGC |
| | | TATGTAGTGCAGAACATTTTCCAGATGTTGGAGGTTTATC |
| | | TTCGTTAGCTCACTTGGATTTGACTAGAAATGATGAGAGT |
| | | ACCAATCAACTCTTTCATTACTTAGACATAACATTTTGTG |
| | | AGAAGCTTGCATTGCCTGAACTTCCGCCATTTATAAAGGA |
| | | GTTATATGCATGTGATCCTCTAATCTTGAAAAGCATCCCT |
| | | GATTTCCTTACCAAATATTCAGATCTCTATTCAGTGTCAT |
| | | TCACTCGCCATATCGAGAACAAAGGTGAACTGACTGATTT |
| | | ATTGCTTTTTGTCCTCCGCATGATTAGTGTGGCATCTCAG |
| | | TGTGAGAAAAGGCCGCCCTTGAGCCTTTTCTTCCCTGGAG |
| | | ATATAAAATGGAAATGGTTCACTTATTATCGGAAAGAACA |
| | | AACGAAAAGATTCTCCACTCAACTTGATCCACGTTGGTAC |
| | | GAAAGTAAATTCATCAAAGGATTTGTTTTATGCTTTCGTG |
| | | TACCATCGGTGATTGGTCAGAACCAGAAATGTTCAAATGA |
| | | TAAATCACAACGAGGTAGTAGTCGTAGGTTCGGATGCACT |
| | | AAGGTTACTGCCAAGTTAGTGCAAAGATATAACAGGCAAG |
| | | AACATGATGTACTCCAGAAAAAATGTCTGATTGTTGCTCG |
| | | CCAAACTTTTTGCTCTCATGGTAGTAAATACTCCATTTGT |
| | | TTTAACTACATACCTTTTGTAGCATGTGAGAAAGGGAAGA |
| | | AGCTGCTAAATGAGTATTGCTTCTTCGAGGCATCGTTAGA |
| | | ACCAGACATTGCAACAAAATGGGGGCTTCTTCTGGTGTAT |
| | | GAGAAGAAAATTCAACAGATAGATCAATCAACCATCGCGG |
| | | TCCAATGTGATGTTGATTCTCCGAATTCTGATCTGTTTAG |
| | | AGAATGTAATGATGACCAAGGCCACAGAAAAGAAGATGTT |
| | | TCTGTTAAGAGAAGACGCCTTGATCTTCGTCAAGTGATGA |
| | | AGGATTCGTGCTCCACCAGCATAGTTCCTGATCAACAAAT |
| | | GGAAACACCAAGCTCTTCTGCAGCACAAAGCTTCCAGCAT |
| | | AGGGATGAGTCATGCTCTTCAGGACAGCCCCAAACTACAC |
| | | AGATCCCTCAAGCTGATGATCCGCGAGTTGATGAAGCAAT |
| | | AGATGAGGCTACCTCATGTGTGGTAGTTGACCAGCCTGAC |
| | | GTTTTTGATGTCTCTCCAGATGAGCACAAAGATAATTCAA |
| | | TGACAACTGGTTCTGGCTGCTCTGTAGTATCCCAACAATT |
| | | GGAGACACAATGCACTTCTGGAAAATCTCAAACTCTCCGG |
| | | CTCTTCCCCGATCATTCATGA |
| SEQ | Solyc01g073710.2 | ATGGGAGTGGTGATTATCGACGGATCAACAATTCGAGATT |
| ID | | TCGTCAACGACGAAGCGGCTTTCACAAAGTCAATTGATAA |
| NO: | | GGGATTTACCGATTTGGACCTCAATAACGACGGCGTTTTG |
| 132 | | TCACGATCCGAGCTCCGCAAAGCCTTCGAATCTCTCCATC |
| | | TCATTGAATCTCACTTCGGCGTCGACGTCGCCGCCACGCC |
| | | GGAGGAACTCACCGACCTCTACGATTCCATTTTCGAGAAG |
| | | TTCGATTGCGACCACAACGGCACTGTCGACCGAGAGGAAT |
| | | TTGGGAACGAGATGAGGAAAATTATGCAGGCGATCGCTGA |
| | | TGGACTTGGTTCATCGCCGATTCAAATGGCGCTTGATGAT |
| | | AGTGAACAGAGTTTGATTAAGCAAGCTGCTGATCTCGAAG |
| | | CTTCTAAGCTTCCTGCCTAA |
| SEQ | Solyc01g087240.3 | ATGCCTACTATTGCTAAACATGATGGTCGGCAGCTGGAGA |
| ID | | CTGGGCTGCGAATTACCTCACTATCAACTACCCAAATGCA |
| NO: | | ACCATGGTGGCACGGTTATGGTGATAATACTATGCCTTTG |
| 133 | | GCTAGTGAGAATGCCGTTGCCCAGGAGAAATCTGAAGGTG |
| | | GCAACCAGGATAAAGAAACAAAGGCTCTAGCCATGGAATC |
| | | TGGATCAGATGGAAATAACGAGCAATACAAGCAGCACCTC |
| | | AAGCATTTTGCACCCACAACTGCTGCCATCATGGCTGAGC |
| | | AGCAGAAGGAACTCACAGGTCATTCAGCTATGTTGGCGTC |
| | | ATATCCATATCCAGATATGCAGTATGGGGGAATGATGACT |
| | | TATGGCGCACCTGTACATCCCCACTTATTTGAAATTCATC |
| | | ATGCCCGAATGCCTTTGCCTCTCGACATGGAAGAGGAACC |
| | | TGTCTACGTGAATGCAAAACAGTACCATGGAATATTAAGG |
| | | CGAAGACAGATAAGAGCTAAAGCAGAGCTTGAGAGGAAAG |
| | | CTATTAAAGCGAGAAAGCCATATCTGCATGAATCTCGGCA |
| | | TCAGCATGCTATGAGAAGGGCAAGGGGAACTGGAGGCCGC |
| | | TTTCTGAATACTAAAAAGCTCAATGACATGGACTGTACAC |
| | | CAACGGAGACTCAAAAATATGGGGCAACCATTGCAACTCA |
| | | CTCTGGCAACTCTTCTGGTTCCGGGAGTTCTGATCAAGGG |
| | | GGAAAGGAAGGATCTACAGTTCAAGACATGCACAAAGGAC |
| | | ACAGCCAGAGTTTCACAACTGGCAATGGCCATGGTTCATC |
| | | TGTGTATTTTTCCGCGAGCAGTGGAAGTGAGCAAGGGAAT |
| | | GGCCATTACGGTCATGGAAGTTGGAGCCTGCTGGTCAATC |
| | | AGGCTTCTCAGGGGGCAGCTTCCAGCAATTAA |
| SEQ | Solyc01g087900.3 | ATGGTGTGCATACGAAAGGCGACGATAGATGATTTGCTTG |
| ID | | CTATGCAAGCATGCAATTTGTTATGCCTTCCTGAGAATTA |
| NO: | | CCAGATGAAGTACTACTTCTACCACATACTTTCGTGGCCT |
| 134 | | CAGCTTCTGTACGTTGCTGAAGATTACAATGGTAAAATTG |
| | | TGGGATATGTGTTAGCAAAAATGGAAGAGGAAACCACTGA |
| | | GTGCCATGGGCACATCACTTCCCTTGCCGTAGTCAGGACT |
| | | CACCGGAAACTTGGTTTGGCTACTAAGCTCATGACCGCTG |
| | | CTCAGAATGCTATGGAACAGGTGTTTGGAGCAGAGTATGT |
| | | GTCTCTGCATGTTAGGAAGAGCAACAGGGCCGCATTTAAG |
| | | CTGTATACAGAGACATTAGGATATAAGATTCATGATATGG |
| | | AAGCCAAGTACTATGCAGATGGGGAAGATGCTTATGATAT |
| | | GAGGAAGGAATTGAAGGGCAAGAAGCACCACCAGCATCAC |
| | | CATCACCACCACCACCACGGTGGTGGGTGTTGCTCTGGTG |
| | | AGGTGAAGGTAGGACCAAAGGAAGGAGCAGCAGAAACAAA |
| | | AGCAGCATAA |
| SEQ | Solyc01g090710.3 | ATGTATTGTGGTAGCATTTTAACAGTGTCTAATTTCAATT |
| ID | | ATTTGTGAATTTTATTCTTAATTATTAAATTAATGGAATA |
| NO: | | TTGGGGTTCAATTGAAAAGCCTATTGTTTTGTTGGGTTGT |
| 135 | | GCATATTTGTCATGGAAGATTGTTACGCATATATGGAGTT |
| | | TCATGAATATAGAAAAGGATCCCATTACAGTGCTTGTTAC |
| | | TGGTGCTGCAGGTCAAATTGGCTATGCTCTTGTTCCTATG |
| | | ATCGCGAGAGGAGCCATGTTAGGCCTTGATCAACCTGTTA |
| | | TCATACACATGCTTGATATTGAACCAGCTGCTGAGTCCTT |
| | | AAAAGGAGTGAAAATGGAATTGATTGATGCAGCTTTTCCT |
| | | CTTCTCAAAGATGTTGTTGCTACCACGAACGTTGTTGAAG |
| | | CTTGTAAAGGCGTCAATGTTGCTGTTATGGTTGGTGGATT |
| | | TCCGCGAAAAGAAGGGATGGAAAGAAAGGACGTGATGACG |
| | | AAAAATGTTTCAATTTACAAAGCACAAGCCTCTGCCTTAG |
| | | AACAGCATGCTGCCCCTGATTGCAAGGTTTTGGTTGTTGC |
| | | TAATCCAGCAAACACTAATGCACTTATTTTGAAAGAATTT |
| | | GCCCCTTCATTTCCAGCGAAAAACATCACTTGCCTAACAA |
| | | GGCTTGATCACAACAGAGCTTTAGGTCAGATTTCGGAGAA |
| | | ACTAAACGTTCACGTTGGTGATGTGAAGAATGTGGCAATA |
| | | TGGGGAAATCACTCATCAACTCAGTATCCAGATGTGAATC |
| | | ATGCAACTGTGAAAACCGCTGCTGGAGAAAAGCCTGTCAG |
| | | AGAACTTGTTGCTAATGATCAATGGTTGAACACGGAATTC |
| | | ATCACCACAGTGCAACAACGAGGAGCCGCTATCATAAAAG |
| | | CTCGAAAGCTATCAAGTGCATTATCTGCAGCTAGCTCAGC |
| | | TTGTGATCATATACATGATTGGGTTCTCGGCACTCCAAAG |
| | | GGGACATGGGTTTCAATGGGGGTTTATTCTGATGGATCTT |
| | | ATGGCATTCCAGCTGGTTTAATTTACTCTTTCCCTGTTAC |
| | | TTGTGAAAAAGGAGAATGGTCAATTGTGCAAGGTCTAAAG |
| | | ATAGACGAGTTTTCAAGGGCCAAGATGGATGCAACCGCGA |
| | | AGGAGTTGGCTGAGGAAAAATCCTTAGCGTATTCATGCCT |
| | | AAACTGA |
| SEQ | Solyc01g091620.3 | ATGAATGTCATGCGTCGCCTTAAGAGCATTGCTTCTGGAC |
| ID | | GTTCTTCTGTTTCAGATCCTGGTGGTGATTCAAGCATAAA |
| NO: | | GAGGGTGAAGGTTGAGAAAGAAGTAGATCAAAGAGTGGTT |
| 136 | | GGTGAAACTCAAATGGAAGAGGGATGTACAACTACTACAG |
| | | TTCCAAAGGAAGACATGGCTTCTACATCTATGGAAACCAC |
| | | TGCTGGAAGTACCTCAACAATGGATATTAGACTTGAAAAT |
| | | TCTGAACTTGATGAACTTCCTAAAGAAATGCATGAAATGA |
| | | AAATTAAGGATGAAAAAGATGATAAAGCTGATAGTCTAGA |
| | | TGATAATTTGAAGGATATGGAACCTGCTGTTGTTAGTGGA |
| | | AATGGAACAGAAACTGGTCAGATTATTGTGACTACTGTGA |
| | | GTGGTAGGAATGGACAAGAGAAACAGACATTGTCTTACAT |
| | | GGCGGAGCGCGTGGTTGGCACTGGTTCATTTGGAGTTGTC |
| | | TTTCAGGCTAAGTGCTTGGAAACTGGTGAATCTGTTGCAA |
| | | TAAAGAAGGTCTTACAGGATAGGAGATACAAGAACAGGGA |
| | | ACTTCAGATTATGCGCACACTTGATCATCCTAATGTTGTT |
| | | AAACTACGACACTGCTTCTATTCTACTACTGAGAAGAATG |
| | | AAGTCTACCTTAACCTTGTCCTGGAATACGTGTCTGAAAC |
| | | TGTTTACCGAGTATCAAGGCACTACAGCAGAATGAACCAA |
| | | CACATGCCCAATATATATGTGCAGCTATACACATACCAGA |
| | | TATGCCGTGCTCTGAATTACATGCATGGTGTCCTTGGTGT |
| | | ATGCCATCGTGATATTAAACCACAGAATCTTTTGGTTAAT |
| | | CCCCACTCGCATCAGCTAAAGCTCTGTGATTTTGGTAGTG |
| | | CAAAGATGTTGGTGCCTGGAGAGCCCAACATATCATACAT |
| | | TTGTTCTCGTTATTATCGGGCTCCTGAATTGATCTTTGGT |
| | | GCTACTGAGTACACAACTGCAATTGACATGTGGTCTGCTG |
| | | GTTGCGTTATGGCTGAGCTACTTTTGGGACAGCCTCTTTT |
| | | CCCTGGAGAAAGTGGCGTTGATCAGCTGGTCGAAATCATC |
| | | AAGATATTGGGGACACCAACAAGAGAGGAGATTAGGTGCA |
| | | TGAATCCAAATTACACAGAGTTCAAGTTTCCCCAGATCAA |
| | | AGCTCACCCATGGCACAAGATATTTCATAAAAGAATGCCC |
| | | CCTGAAGCAGTTGATTTGGTGTCGAGGCTTCTCCAATATT |
| | | CTCCAACTCTACGCTGCACTGCTTTGGAAGCATGTGCACA |
| | | CCCTTTCTTTGATTCTTTAAGGGAACCAAATGCTTGCTTG |
| | | CCAAATGGGCGACCTCTGCCTCCCCTATTCAACTTTTCAC |
| | | CTCAAGAGCTCTCTGGCGTGCCTGCTGAACTGCGAAAGCG |
| | | CCTTATTCCTGAACACTTGCGCAAGTGA |
| SEQ | Solyc01g095410.3 | ATGCCGAAGAATAAGGGAAAGGGAGGAAAGAACAGGAAGA |
| ID | | GAGGGAAGAACGAAGCTGATGATGAAAAGAGAGAGCTTGT |
| NO: | | TTTTAAGGAAGATGGACAGGAATATGCTCAAGTTCTTCGT |
| 137 | | ATGCTTGGAAATGGCCGATGTGAAGCTATGTGTATTGATG |
| | | GGACGAAACGTCTATGTCACATACGTGGTAAGATGCATAA |
| | | GAAAGTTTGGATTGCCGCCGGTGATATCATCCTTGTCGGA |
| | | CTCCGTGATTATCAGGATGACAAGGCTGATGTTATTCTGA |
| | | AGTATATGCCAGATGAGGCAAGGTTGTTGAAGGCATACGG |
| | | AGAGTTACCAGAGAACACTAGGCTCAATGAGGGTATTGCT |
| | | AATTTGGATGAAGAAGATGAGAATGCCGCTGATGATTACA |
| | | TTGAGTTTGAAGATGAAGACATTGACAAAATCTAA |
| SEQ | Solyc01g095700.3 | ATGGAATCACAGTTTATTGAAAGATACCATAGTCATCAGC |
| ID | | CTAGTGAACATCAGTGTTCTTCATCTCTTGTTAAACACAT |
| NO: | | CAAAGCACCAGTTGATATTGTTTGGTCACTAGTGAGGAGA |
| 138 | | TTTGATCAGCCTCAGAAGTATAAGCCATTTATTAGCAGGT |
| | | GTACTGTGAAGGGTGATCTTACAATTGGTAGTGTTAGAGA |
| | | GGTGAATGTCAAGTCTGGTCTTCCAGCCACCACTAGCACT |
| | | GAAAGATTGGAACTTCTTGATGATGAGGAGCACATCCTTG |
| | | GCATCAGAATCGTTGGTGGCGATCACAGGCTAAAGAACTA |
| | | TTCTTCGGTTATTACAGTCCATCCAGAGACACTTGATGGT |
| | | AGACCGGGGACACTGGTGATTGAGTCATTTGTAGTGGATG |
| | | TGCCTGAAGGGAACACTAAAGAGGAGACTTGCTACTTTGT |
| | | GAAGGCCTTAATTAATTGCAATCTGAAATCACTAGCTGAT |
| | | GTATCTGAGAGGATGGCTATGCAGGGAGGTGTTCTACCCG |
| | | TCAGTGTGAACTGGTCTTCGAGTAACCAGATCGAGATATG |
| | | A |
| SEQ | Solyc01g096580.3 | ATGGCAGCATATGCAGCAATGAAGCCAACCAAGCCTGGGC |
| ID | | TTGAGGAGCCTGCAGAGATGGTTCACAAGATTAGGATCAC |
| NO: | | TCTCTCCTCCAAGAATGTTAAGAATCTCGAGAAAGTGTGC |
| 139 | | GCTGATTTGGTTCGTGGTGCCAAGGATAAGAGGCTCAGGG |
| | | TGAAGGGACCAGTGAGAATGCCCACAAAGGTCCTTAACAT |
| | | CACCACTAGAAAGTCTCCTTGTGGAGAAGGTACAAATACA |
| | | TGGGACAGATTTGAGCTTCGTGTCCACAAGCGGGTGATTG |
| | | ACCTTTTCAGCTCACCAGATGTTGTGAAACAAATCACTTC |
| | | AATCACCATTGAACCCGGTGTTGAGGTTGAGGTCACTATT |
| | | GCTGAATCTTAG |
| SEQ | Solyc01g096760.3 | ATGGACGGCGGCAGCAGCAACGCACCAGTTCAGCAGCTGA |
| ID | | CGGCGGAGGCAGCTGCACAGCTACAGGAAGGTATCGGTTT |
| NO: | | GGTGTTAACTAGATGGACTGCTCTTCAGATGGCTATCGAG |
| 140 | | AACGAGTGGGGTGGCCGAGATACTCGTGAGAAATCCAATC |
| | | AACTCAATGTTGACATTTTTTCCGCTTTTACACAATCCAA |
| | | AGAAAAAGTATACATGGATGATATAGAAGAAATTCTAGAT |
| | | GAGTTCATGATATCTCTAAATACTGAAGTCAATGATGGCA |
| | | GCCTTGAGGAGGTAGCAGAAAAATTGATGTATATGCATGA |
| | | AGAGTGTCTGGAAGGGGATTTCAATTCAATCAAAGTGCTA |
| | | AGGGAAACAAATGTTGGGAGGAGACCTGCTACTTATGTCA |
| | | GACAAGATGCTAGTGATGACGATGACAGTAGTAATGATGG |
| | | GGATGACCGAAATGAGAACTTTGGCAATAATTCATCCGAT |
| | | ATGGCAATTGATTCTATGGAAAGCCAGCCAAGTTTGGGTC |
| | | AGGATATGATAGTTGAACCCGTGAGAAAGCAGCCAGCTGA |
| | | AGTGGACCCAGACGGGTGGACGACAGTATCTACAAAGCGT |
| | | AACAAAGGTAGAAGGAATTAA |
| SEQ | Solyc01g097520.4 | TCATCTATCAGCTCTCCTCTCTCTCACAGGCACACAAAGA |
| ID | | GACAAAAACTCAAAAACAAAGAAAATTAACAGAAAAGAAA |
| NO: | | AATGGCAGAGGCTAATTCGTATGCAGCTCTTACCAAAGCT |
| 141 | | TTCTCAGGACTTGGTGTGGATGAGAACTTATTCATCTCAA |
| | | CACTGGGGAAATGGAATCGGCATCAGAGGGAATCGTACAG |
| | | GGTAAGCACTCCTGGATTCTTCAGAGAAGACAAACGACAG |
| | | TTCCAGAGATGGGATGATCAACATATCCTCCAGCTTCGTC |
| | | AAGAGTTTTTGCGTCTTAAGGATGCAGTGGTTTTGTACAC |
| | | AATGCACCCATGGGAAAGAGATGCTCGTTTGTTCAAGGAA |
| | | GCATTATTATTGCAAGTACCTAAACATGATGTTCTCATTG |
| | | AGATTGCTTGCACTCGATCCTCTGAAGATCTTCTTGGAGC |
| | | CAGAAAAGCTTACCATTCCCTTTTTGAACACTCAATTGAG |
| | | GAAGACATTGCCTTCCATGTTCGAAGTTCTGAAAGAAAGC |
| | | TTCTAGTTGCTCTTGTAAGTTCCTACCGTTATGAAGGTCC |
| | | ACGAGTAAACGATGATCTTGCAAAATCTGAGGCTAAGATT |
| | | TTTGTAAATGCAATAAAAAATGCCAACAAGAAGAAGCTGA |
| | | TTGATGAGGATGAAGAGATTGTGAGGATCCTTTCGACAAG |
| | | AAGCAAGCTCCATCTTAAGGCCATCTATAGCCATTACAAG |
| | | GAAATCACTGGCAACTTCTTGGACGAGGATCTTGAGTGTG |
| | | ACTTGACCTTGAAACAAGTTGTTCAATGCCTTTGTATACC |
| | | TAAAGCATACTTTAGCAAGATTTTGATCGCGTCCCTAAGA |
| | | TTGGATGTGGATGAATCTGCTAAAGACTCAGTTACCCGAG |
| | | TCATAGTGTCAAGAGCAGACGATGATGATATGCAGCAAAT |
| | | CAAAGAAGAATTTCAGAGCAAATATGGAACTACTTTGGCT |
| | | GAAAAGATTGCAGAAGTTGCTAATGGAAGCTACAAGGATT |
| | | TCTTGCTTACTATAATTGCAAAATCTGAGTAA |
| SEQ | Solyc01g100870.3 | ATGGGTTTATCATTCGGGAAGCTTTTCAGCCGGCTGTTTG |
| ID | | CCAAGAAGGAGATGCGTATTCTGATGGTTGGTCTTGATGC |
| NO: | | AGCTGGTAAAACCACCATATTGTACAAGCTCAAGCTGGGA |
| 142 | | GAGATTGTTACCACCATTCCTACCATTGGATTCAATGTGG |
| | | AGACTGTTGAATACAAGAACATAAGCTTCACCGTCTGGGA |
| | | TGTCGGGGGTCAGGACAAGATCAGACCATTGTGGAGGCAT |
| | | TACTTCCAAAACACACAAGGACTTATCTTTGTGGTTGATA |
| | | GTAATGATCGTGATCGTGTTGTTGAGGCCAGAGACGAGCT |
| | | GCATCGGATGTTGAATGAGGATGAACTGAGAGATGCTGTG |
| | | CTGCTTGTGTTTGCTAACAAGCAAGATCTTCCAAATGCTA |
| | | TGAATGCTGCTGAGATCACTGACAAGCTTGGTTTGCACTC |
| | | CCTCCGTCAACGTCACTGGACAGGTACATTCAGAGCACAT |
| | | GTGCTACCTCTGGAGAAGGGTTGTATGAGGGGCTTGACTG |
| | | GCTCTCAAACAACATTGCAAACAAGGGTTGAACACGAGAT |
| | | CCATGATTTTTGCAGCTATATTTTTGTCAATATAGACTTC |
| | | AAACAATGTCTAGAGCACTTGTGCACCTGCTGA |
| SEQ | Solyc01g101060.4 | ATGGAAACTTTCTTATTCACCTCCGAGTCTGTGAACGAGG |
| ID | | GTCACCCAGACAAGCTCTGTGATCAGATCTCTGATGCAGT |
| NO: | | TCTTGATGCCTGCCTTGAGCAAGATCCCGAGAGCAAAGTT |
| 143 | | GCATGTGAAACTTGCACCAAGACCAACTTGGTCATGGTCT |
| | | TTGGTGAGATCACAACCAAGGCTATTGTAGACTATGAGAA |
| | | GATTGTGCGTGACACATGCCGTAATATTGGATTTGTTTCT |
| | | GATGATGTTGGTCTTGATGCTGACAACTGCAAGGTCCTTG |
| | | TTTACATTGAGCAGCAAAGTCCTGATATTGCTCAAGGTGT |
| | | CCACGGCCATCTGACCAAACGCCCCGAGGAGATTGGTGCT |
| | | GGTGACCAGGGCCACATGTTTGGCTATGCAACAGATGAGA |
| | | CCCCTGAATTAATGCCTCTCAGTCACGTGCTTGCAACTAA |
| | | ACTTGGTGCCCGTCTTACAGAAGTCCGCAAGAATGGCACC |
| | | TGCGCCTGGTTGAGGCCTGATGGCAAGACCCAAGTTACTG |
| | | TTGAGTATAGCAATGACAATGGTGCCATGGTTCCAATTAG |
| | | GGTACACACTGTTCTTATCTCCACCCAACACGATGAGACC |
| | | GTTACCAATGATGAGATTGCCCGCGACCTTAAGGAGCATG |
| | | TCATCAAACCAGTCATCCCAGAGAAGTACCTTGATGAGAA |
| | | TACTATTTTCCACCTTAACCCATCTGGCCGATTCGTTATT |
| | | GGTGGACCTCATGGTGATGCTGGTCTCACTGGTCGTAAAA |
| | | TCATCATCGACACTTATGGTGGTTGGGGTGCTCATGGTGG |
| | | TGGTGCTTTCTCGGGCAAAGACCCAACCAAGGTCGACAGG |
| | | AGTGGTGCATACATTGTAAGGCAGGCTGCAAAGAGTATCG |
| | | TAGCTAGTGGACTTGCTCGTAGATGCATCGTGCAGGTATC |
| | | TTATGCCATCGGTGTGCCTGAGCCATTGTCTGTATTCGTT |
| | | GACACCTATGGCACTGGAAAGATCCCTGACAGGGAAATTT |
| | | TGAAGATCGTTAAGGAGAACTTTGACTTCAGACCTGGAAT |
| | | GATGTCCATTAACTTGGATTTGAAGAGGGGTGGCAATAGA |
| | | AGATTCTTGAAAACTGCTGCCTATGGTCACTTTGGACGTG |
| | | ATGACCCCGATTTCACATGGGAAGTTGTCAAGCCCCTCAA |
| | | GTGGGAAAAGCCCCAAGACTAA |
| SEQ | Solyc01g103490.3 | ATGGCGTTGAAGTTATCTGCTGCTGAAGCAGCTAAGGTTG |
| ID | | CGATTCAGTCTATAGGATGTGGATATGATATATCATTGGA |
| NO: | | TTTGAGGCTCAAGTATTGTAAAGGTCATCCAGATAATAGT |
| 144 | | CGTTTAATTGAAATTGATGAAAATGAGAGTGGGGAGGTTG |
| | | TGCTTCCTGGTGGAATTTCAATACCTAATGTGTCAAAATC |
| | | TATCAAATGTGATAAAGGAGAACACACACGGTTTGGCTCT |
| | | GATGTTCTTTCTTTCCAGCAGATGTCAGAGCAGTTCAATC |
| | | AGGAAGTATCCGTGAATGGTAAAATTCCTTCGGGGCTCTT |
| | | CAATACTATGTTCGAGTTTTCTGGTTCTTGGCAGAAGGAT |
| | | GCTGCATACACTAAAACCCTTGCTTTTGATGGTGTGTTCA |
| | | TCACGTTGTACTCTGTTGCTCTAGAAAAATCTCAAATGGT |
| | | GCTGTGTGATTATGTTAAAAAGGAAGTTCCATCAACATGG |
| | | GATCCTGCAGCATTAGCAAGGTTTATTGAAAAATTTGGTA |
| | | CTCATGTCATTGTTGGTATAAAGATGGGGGGAAAAGATGT |
| | | AATATATATGAAACAACAGCACTCCTCATCGCTTCTGCCC |
| | | GCTGACGTGCAAAAACGTTTGAAGACAATGGCAGATAAGA |
| | | GGTTTTCAGGTGCAAATGAACAAGGGGGAGTAGAATCGCA |
| | | GCAGACACATCAGAATGAGAAGTTTGAAATCAGGGAGCAC |
| | | CGACTGAGGTTTGCCGATCCCAATATATCAGGTTCATATG |
| | | CACATAAGGAGGATACTGTTTGTATTTGCAAGAGGAGAGG |
| | | TGGAAGTGATAGCAGAGGCCTGATGCATAATCAGTGGTTA |
| | | CAAAGTGTTGATCAGGAGCCTGATGTGATCTCAATGTCCT |
| | | TCATTCCTATAACTTCACTGTTGAATGGCATCTCAGGAAG |
| | | TGGATTTTTAAGTCATGCTATAAATTTGTACTTACGCTAT |
| | | AAACCACCAATTGAAGAGCTTCACCAGTTTTTGGAATTTC |
| | | AGCTGCCAAGGCAATGGGCACCAGTATTCGGTGATCTTCC |
| | | GCTTGGTCCACAGAGAAGGCATTTAAGTTTTCCGTCTTTG |
| | | CAGTTCAGTTTACTGGGTCCCAGGCTTTATGTGAACACCA |
| | | CTCCGGTTGATGTTGGCAAGAGGCCGGTGACTGGACTTCG |
| | | TTTGTACCTTGAAGGTAGAAGGAGTAACCGCTTAGCCGTC |
| | | CATTTACAGCATCTTTCATCTCTTCCTAAAGTCTTCCAAC |
| | | TTGAAGATGATCCAAAAGGCAACTTTCGCCGAGAATCATA |
| | | TGATCGCAAATACTATGAAAAAGTTCAATGGAAGAACTTT |
| | | TCTCACGTTTGCACCGCACCTGTTGAATCTGATGAAGACC |
| | | TTAACATAGTTACTGGAGCACAATTGGAATTAGGGGATTA |
| | | TGGGTTTAAAAAGGTCCTCTTTCTGAGACTCCGTTTCTCG |
| | | ACTGTCATGGGAGCTACTATGGTAAAGCACGCTGAGTGGG |
| | | ATGGTTCCCCTGGGTTGGCTCGCAAATCTGGCCTGATATC |
| | | AACTCTCATTAGCCAGCACTTTACATCAGTTCAGAAGCCA |
| | | CCTCCTCGGCCAGATGATGTGAACATAAATTCTGCCATTT |
| | | ACCCTGGTGGTCCTCCAGTTCCTGTGCAAGCCCCTAAGCT |
| | | CTTGAAATTTGTCGATACTACTGAGATGACTCGAGGACCA |
| | | CAGGAACCTCCTGGTTATTGGGTTGTTTCAGGTGCAAGGC |
| | | TTATGGTTGAGAAAGGAAGGATCTCTGTGAGGGTCAAATA |
| | | TTCCTTGCTAACTGTAATACAACCCGATGATGAAATATCA |
| | | GAGTAA |
| SEQ | Solyc01g105070.3 | ATGGCGTATTACAATTACAATTTAATCAACAAAATGGTTA |
| ID | | CAATTATTTTTATTCTGGTGCTAGTAATTGTTGATGTGAC |
| NO: | | TATGGTATTCGGCCAAGGGACTCGTGTTGGATTTTATTCC |
| 145 | | AGTACGTGCCCAAGGGCCGAATCCATAGTTCAATCAACGG |
| | | TGAGGTCTCATTTTCAGTCTGATCCAACAGTGGCACCAGG |
| | | ATTGTTGAGAATGCACTTTCATGATTGTTTCGTACAAGGT |
| | | TGTGACGGTTCCATCCTCATTTCTGGTACTGGCACTGAGA |
| | | GAACAGCTCCTCCGAATTCCAATTTGAGAGGATTCGAGGT |
| | | TATTGACGATGCTAAGCAGCAAATTGAAGCTGTTTGTCCT |
| | | GGAGTTGTTTCATGTGCTGACATTCTTGCTCTTGCTGCTC |
| | | GTGATTCCGTTCTCGTGACTAAAGGATTGACCTGGTCTGT |
| | | CCCCACGGGACGCAGAGATGGACGAGTTTCAAGCGCATCA |
| | | GACACATCTAATCTGCCAGGTTTTACTGAATCTGTTGATG |
| | | CTCAAAAGCAAAAGTTTGCTGCAAAGGGTCTCAACACTCA |
| | | AGATCTTGTCACCCTTGTTGGTGGGCACACAATTGGAACC |
| | | TCAGCATGCCAATTCTTCAGCTACAGGCTATACAATTTCA |
| | | ACTCCACTGGTGGCCCTGACCCTTCAATAGATGCAACCTT |
| | | TCTTTCTCAGCTTCAAGCATTATGTCCACAAAACGGAGAT |
| | | GGCTCGAAGCGTGTGGCACTGGACACTGGAAGCGTGAACA |
| | | ATTTTGACACCTCGTATTTCTCTAACTTGAGGAATGGTCG |
| | | GGGAATTCTGGAATCAGACCAGATATTGTGGACCGATGCT |
| | | TCGACCAAGGTGTTTGTCCAAAGGTATTTAGGCCTCAGGG |
| | | GATTTCTTGGATTGAGATTCGGCTTAGAATTTGGAAAGTC |
| | | CATGGTTAAAATGAGCAATATTGAAGTTTTGACAGGGACT |
| | | AATGGTGAAATTCGTAAAGTTTGCTCTGCATTCAACTGA |
| SEQ | Solyc01g105340.4 | ATGCTAGCAAATCAGAAATCAAAAGCTGTGACTTATCGAA |
| ID | | AGCTTGCAAGGAGTTACCATCCAGATGTGAACAAAGAACC |
| NO: | | TGGAGCTGAACAGAAATTTAAGGAGATCAGCAATGCGTAT |
| 146 | | GAGGTTTTGTCTGATGATGAGAAACGTTCCATATATGATA |
| | | AATATGGGGAGGCTGGTCTTAAAGGAGCTGGTGCGGGCAT |
| | | GGGGGATTTTAGCAACGCCTTTGATTTATTCGAGTCTTTG |
| | | TTCGACGGCTTTGGTGGTATGGGTGGCATGGGTGGTATGG |
| | | GAGGTAGAGGTTCTCGGAGCAGGGCCACAGAAGGTGAAGA |
| | | CCAGGGCTATAATCTGGTTTTGAATTTCAAAGAAGCTGTA |
| | | TTTGGGGTTGAGAAGGAGATTGAAATTAGCAGGCTTGAAA |
| | | CTTGTGGTACCTGTGATGGATCAGGTGCAAAACCTGGGAC |
| | | TAAACCGTCGACCTGTAATACTTGTGGTGGGCAAGGGCAG |
| | | GTTGTGTCCTCAGCAAGGACCCCACTGGGTGTTTTCCAGC |
| | | AGGTGACAACATGCTCTTCTTGTGGGGGGACTGGAGAGAT |
| | | TTCTACTCCTTGCGGCACCTGCAACGGTGATGGCCGAGTG |
| | | AGGAAGTCAAAACGCATTAGCTTGAAAGTTCCACCTGGTG |
| | | TAGATTCCGGTAGCCGTTTAAGGGTTCGTTCAGAAGGTAA |
| | | TGCAGGAAGGAGAGGTGGACCCCCCGGAGATCTTTTTGTC |
| | | ATGATTGAAGTCCTCCCGGATCCAGTATTAAAACGGGATG |
| | | ACACCAATATTCTCTACAATTGCAAAGTTTCTTACATTGA |
| | | TGCGATATTGGGTACCACTATGAAGGTTCCTACAGTAGAT |
| | | GGGATGGTTGATCTAAAGATTCCGGCAGGTACCCAGCCAG |
| | | GGACAACATTGGTGATGGCCAAAAAAGGGGTGCCATTCCT |
| | | GAGCAAACCAAACATGAGAGGGGATCAATTGGTGAGAGTG |
| | | CAAGTTGAGATTCCAAAACGGTTGAGTAGTGAAGAAAGAA |
| | | AGCTCATAGAAGAACTTGCCAATCTAAACAAGCCTAAAGC |
| | | TGCTACCAACAGCAAGAGGTAG |
| SEQ | Solyc01g109660.2 | ATGGCCGACGTTGAATACAGGTGCTTCGTCGGTGGTCTGG |
| ID | | CATGGGCCACCACCGACCAAACACTTTCGGAAGCTTTTTC |
| NO: | | TCAGTACGGCGAAGTGGTCGAATCCAAGATCATCAATGAC |
| 147 | | CGGGAAACTGGTAGATCTAGAGGATTTGGATTCGTTACCT |
| | | TCAAGGATGAGCAAGCCATGAGGGATGCTATTGAAGGGAT |
| | | GAACGGCCAGGATCTTGATGGTCGCAACATCACCGTGAAC |
| | | GAAGCTCAATCACGCGGAGGCGGTGGAGGTGGAGGTGGAA |
| | | GAGGCGGGGGTGGTTACGGAGGTGGCCGACGTGAAGGCGG |
| | | TGGCGGAGGCTACGGTGGTGGCGGCGGCTACGGAGGTGGC |
| | | CGACGTGAAGGCGGCGGCGGCGGCTACGGTGGAGGCGGCT |
| | | ACGGAGGTGGTCGTCGTGAAGGTGGTTACGGTGGAGGCGG |
| | | TGGTGGTTATGGAGGAGGTGACCGCTACAACGATCGCTCT |
| | | TCAAGAGGTGGAGGCGGTGGTGGTTCCGATGGGAACTGGA |
| | | GGAATTAG |
| SEQ | Solyc01g111330.4 | ATGGATTTTTCATATTCTTCCCCTTCACATTTCATTCTTA |
| ID | | GTTTATGTTTTGCTCTCCTCATTTTATCCAGAGGTGTTTT |
| NO: | | AGGGGCTACATTTACATTTGTGAACAAATGTGAGTACACA |
| 148 | | GTATGGCCAGGGATTCTAGCAAATGCAGGTAGTCCAACTC |
| | | TGCAAAGTACTGGATTTGAGCTTCCTCAAGAGTCTTCACG |
| | | TGTATTCACAGCACCCACTGGTTGGTCCGGTAGGTTTTGG |
| | | GGACGAACCAGTTGTACGTTTGAGGGATCCGGGTCGGGTT |
| | | CTTGCGGAACAGGGGATTGCGGCTCCGGTGAGGTAGAATG |
| | | TAACGGTGCTGGAGCTGCTCCGCCTGCTACTCTAGCGGAG |
| | | TTTACATTGGGTACTGTTGGGCAGGATTTCTATGACGTGA |
| | | GTTTGGTGGACGGGTACAATTTACCTATGGTGGTAGAAGC |
| | | GTCGGGCGGGTCGGGTATGTGTGCGACAACGGGTTGCGTA |
| | | ACGGATCTGAATCAAATCTGCCCGTCGGAGCTGAGGAGTG |
| | | GCGGTGGAGAGGCATGTAGAAGCGCGTGTGAGGCGTTTAA |
| | | CAAGCCGGAGTATTGCTGCAGCGGCGCGTACAATACACCG |
| | | TCCACTTGTAAACCGTCAATGTACTCACAGATGTTCAAAT |
| | | CTGCGTGTCCAAGATCATACAGCTACGCTTATGATGATCC |
| | | TACCAGTACTTTCACTTGCAGTGGTGCAGATTATACCATC |
| | | ACATTTTGTCCCTCTACCCCAAGTCAAAAATCTACAAAAG |
| | | ATACTACTACCCCCAATGGTGATGGGTCATATGGTGCTAC |
| | | TGGTACAGGAGTTGTGGACCCTGCAACAGGAGGAATCGAG |
| | | TCAAGTTCGGGTCAAATGGGAACCGGATCAGGCACCGGGT |
| | | TTGAGTTTGGTGCAGGAACAGGATATGGGTCAGGAACCGA |
| | | AACACAGCCTGGATCGGGTTCACAAGCTATGCTAGCAGAT |
| | | GGATCCTATTTAGCTGGACTGGCTATGGGAGAATCAAGTA |
| | | GGGCATTTTTTCCATTGAATTTAGCAGGCAGTGCTTTGGC |
| | | TACAACTTTGGTTTCATTAGTTTTCTTGTAG |
| SEQ | Solyc01g111450.3 | ATGGCTAGATACGACAGAGCGATCACCGTTTTCTCGCCGG |
| ID | | ACGGCCACTTATTCCAAGTGGAGTACGCCATGGAAGCCGT |
| NO: | | ACGCAAAGGAAACGCCGCCGTCGGTGTACGTGGAACTGAC |
| 149 | | ACTGTTGTCCTCGGCGTCGAGAAGAAGTCCACTCCCAAGC |
| | | TTCAAGACTCCAGGTCAGTAAGAAAGATAGTGAATCTAGA |
| | | TGATCACATTGCGTTGGCGTGTGCTGGGCTGAAAGCAGAT |
| | | GCACGAGTTCTTGTGAATAAAGCACGCATTGAGTGTCAGA |
| | | GTCATAGGCTTACAGTTGAAGATCCTGTTACTGTTGAGTA |
| | | CATAACTCGTTACATTGCTGGTCTTCAGCAAAAGTACACC |
| | | CAAAGTGGTGGGGTGAGGCCATTTGGTCTATCCACCTTGA |
| | | TCATTGGTTTTGACCCACACACAGGTGTCCCCTCACTTTA |
| | | CCAAACAGATCCATCAGGTACATTCTCAGCTTGGAAAGCC |
| | | AATGCGACTGGTAGAAACTCCAACTCTACTCGGGAATTCT |
| | | TGGAGAAGAATTACAAAGAAACATCTGGCCAGGAAACTGT |
| | | GAAACTAGCAATACGTGCATTGCTTGAAGTTGTTGAAAGT |
| | | GGTGGAAAAAACATAGAAGTTGCTGTGATGACAAAAGAGC |
| | | ATGGGCTTAAGCAACTTGAGGAAGCTGAAATTGATGCCAT |
| | | TGTTGCTGAGATCGAAGCAGAGAAAGCTGCTGCAGAAGCA |
| | | GCCAAAAAGGCCCCACCGAAGGAAACCTAG |
| SEQ | Solyc01g111520.3 | ATGGGTTTTGTGAGTACGATTTTGGGTTTTTGTGGATTTG |
| ID | | GAGTTGGGGTTTCATGTGGATTGACGATCGGTTACTATCT |
| NO: | | CTTCATCTATTTCCAACCCTGTGATGTTAAGGACCCTGTT |
| 150 | | ATTCGTCCATTGGTTGAGCGAGATTCTAAAAGCTTGCAGC |
| | | AGTTGCTGTCTGAAATTCCTCTCTGGGTCAAATGTCCAGA |
| | | TTATGACCGTGTGGACTGGCTCAACAAATTTATCGAGTAT |
| | | ATGTGGCCTTACCTGGACAAGGCAATTTGCAGGACTGCAA |
| | | AAGATATTGCGGCACCAATTATTGCTGAACAAATACCGAA |
| | | GTATAAAATTGATTCTGTTGAATTTGAGACACTAACTTTG |
| | | GGGTCCTTACCCCCTACTTTCCAGGGGATGAAGGTCTATG |
| | | TTACTGAAGAAAAAGAATTGATCATGGAACCATCAATAAA |
| | | GTGGGCTGGAAATCCTAATGTTACCGTTGCGGTCAAAGCA |
| | | TTCGGATTGAAAGCAACTGTTCAGGTTGTGGACTTGCAGG |
| | | TTTTTGCAGCTCCACGTATTACTCTGAAGCCTCTGGTTCC |
| | | AAGCTTTCCATGTTTTGCCAATATCTTTGTGTCCCTCATG |
| | | GAAAAGCCACATGTTGACTTTGGATTGAAGCTTTTGGGGG |
| | | CTGATCTTATGTCCATCCCTGGCTTGTACAGGTTTGTCCA |
| | | GGAGACTATTAAAGATCAGGTTGCTAACATGTATCTTTGG |
| | | CCTAAATCTCTAGAAGTGCAGATATTGGATCCATCCAAAG |
| | | CAATGAAAAAACCTGTTGGGGTCCTGCATGTGAAGATTCT |
| | | GAGGGCTATGAATCTGAAAAAAAAAGATCTACTGGGTGCA |
| | | TCTGATCCTTACGTAAAACTTAAGCTCACTGAGTCAAAAC |
| | | TTCCTTCAAAGAAAACCCCTGTAAGGCATAAGAATTTAAA |
| | | CCCAGAGTGGAATGAGGAATTTAATATGGTTGTTAAAGAT |
| | | CCAGAATCGCAAGCATTGGAGCTCTCTGTTTATGATTGGG |
| | | AGAAGATTGGCAAGCATGATAAGATGGGCATGAACGTTAT |
| | | TCCACTGAAAGATTTGACTCCTGATGAAACAAAAACGATG |
| | | ACACTGAGTCTCCTAAAGAACATGGATGCGAATGATTCTC |
| | | AAAATGATAAGGACCGTGGTCAGATCATGGTGGAATTAAC |
| | | CTACAAACCATTTAAGGAGGATGAGTTGCCAAAAGATTTT |
| | | GAAGATAATGATGCAGCACATAAGGTTCCAGAAGGAACAC |
| | | CACCAGGGGGAGGCGTCCTTATGATTATTGTCCACGAAGC |
| | | TCAAGATGTTGAAGGAAAGCACCATACTAATCCATATGTC |
| | | AAGATTCTTTTCAAAGGAGAGGAGAGAAAAACTAAGCAAG |
| | | TCAAGAAGAACAGGGATCCAAGATGGGAAGAGGAGTTTAC |
| | | TTTTGTGTTGGAGGAGCCTCCTGTGAATGATAGGGTGCAT |
| | | ATGGAAGTTGTCAGCACCTCTACAAGGATTGGCCTATTGC |
| | | ATCCTAAGGAGACATTGGGTTATGTTGATATAAATCTTTC |
| | | CGATGTTGTTAGCAACAAAAGGATCAATGAGAAGTACCAC |
| | | CTCATTGATTCAAAGAACGGTCGCCTTCAAGTCGAGCTGC |
| | | AATGGCGAACCGCATCTTGA |
| SEQ | Solyc01g112000.4 | TACAGCAAGAAAATAAATATGTCTCTCTTCTTCCTTGTCT |
| ID | | TGCTTCTTTTCGTCTCTTCAGTTACAGCCTGTGATCGCTG |
| NO: | | TGTTCACCGATCCAAAGTTGCTTATTTTTCCAGTTCTTCC |
| 151 | | GCGCTTCTCTCGGGTGCTTGTGGTTATGGAAATTTAGCAA |
| | | TAGGATTCAATGGAGGTCGACTTGCCGCTGCTGTTCCTAA |
| | | ACTTTACGATAATGGTGCTGGTTGTGGTGCTTGTTATCAG |
| | | ATAAGATGTACGGACCCAAAAATTTGTTCAAAACATGGAA |
| | | CAACTGTGATGGTTACTGATCTAAATACGAATAACCAGAC |
| | | TGATTTTGTCATCAGTAGCAGAGCTTTTATGGCTATGGCC |
| | | ATTGAGGGAAAAGCTCAAGATGTTCACAAATTGGGAATCG |
| | | CCGATGTTGAATACAAAAGAGTTCCTTGTGACTACAAAGG |
| | | CAAGAATTTGGCTGTTCGAGTAGATGAATCAAGCCAGAAG |
| | | CCGCATTACCTAGCAATTAGCATCTTCTACCAGGGTGGTC |
| | | AAACTGAAATTGTCAGTGTTGATGTTGCTCAGGTTGGATC |
| | | GTCGAACTGGAACTTCTTGAGCCGAAACCACGGAGCAATT |
| | | TGGGATACAAGCAGAGTGCCAAGTGGGGCATTGCAATTCA |
| | | GGTTTGTAGTGACAGCAGGGTACGATGGCAAGTGGATTTG |
| | | GGCTAAATCAGTGCTGCCATCAGATTGGAAAAATGGGGTG |
| | | GTTTATGATACTGGACTTCAAATCACTGACATTGCTCAAG |
| | | AAGGTTGTTCCCCATGTGATGATGGAAGTTGGAAACTTAA |
| | | TTAG |
| SEQ | Solyc02g021640.3 | ATGGCATGTATGAAGTTGGGATCCAAAACAGATGCATTCC |
| ID | | AAAGAAAAGGCCAGGCCTGGTTCTGCACAACTGGCCTCCCA |
| NO: | | AGTGACATTGTTGTTGAAGTTGGGGAAATGACTTTCCATCT |
| 152 | | CCACAAGTTCCCATTGCTTTCTAGAAGTGGGGTTATGGAAA |
| | | AACGGATTGCAGAAGCATCTGAAGGAGAAGATGGCTGTGT |
| | | TATTGAACTCAATGACATCCCTGGTGGTGCTAAAACATTT |
| | | GAACTAGTAGCAAAATTTTGTTATGGGGTTAAACTAGAGC |
| | | TTACAGCAGGTAATGCTGTATACCTTCGCTGTGCCGCAGA |
| | | GCATCTTGATATGACTGAAGAATATGGGGAGGGGAATCTG |
| | | ATATCACAGACTGAGATTTTTCTGAATCAGGTAGTTCTCC |
| | | GTAGCTGGAAAGACTCTTTAAAGGCACTCCAAACCTGTGA |
| | | TGATGTTCTTCCATATGCTGAAGAACTCAGAATTACAAAA |
| | | AGGTGCATCGATTCATTAGCTGTTAAGGCATGTACTGATC |
| | | CGAACCTGTTTGGTTGGCCTGTGATGGAGCACGTTGGCCC |
| | | CTTGCAGAGTCCTGGAGGGAGTATCCTATGGAATGGAATA |
| | | AGCACTGGAGCTAGGCCAAAACATTCAAGTTCAGATTGGT |
| | | GGTATGAGGATGCATCAACTTTAAGTTTACCTCTTTACAA |
| | | GAGGCTAATTTCTGCTATGGAATCTCAAGGCGTCAAACAG |
| | | GATATTGTTGCTGGTTCCCTCAGTTATTATGCTAAAAAGT |
| | | ACCTGCCTGGGCTAAACAGGCGGCAGACCTCTTCTGAGTC |
| | | CACTAACCGACTTGCACCAGTTGGTTTAGGTTCTTCTCTA |
| | | TCAGAAGAAGATCAGAAGCTTTTACTTGAAGAGGTGGATA |
| | | ATTTACTTCCCATGCAAAAAGGCCTAGTTCCAACTAAATT |
| | | CCTCTTTGGCCTACTGAAAACAGCTTTGATTCTTCGAGCC |
| | | TGTCCCTCTTGCATATCAAACTTGGAGAAAAGGATAGGAA |
| | | TGCAGCTTGATCAGGCCACTCTAGAAGATCTCTTAATGCC |
| | | TAATTTCTCTTACTCGATGGAAACACTTTATAATGTTGAC |
| | | TGCGTACAGAGAATTCTAGAGCACTTCTTAGCCATGGATC |
| | | AAGGAACTGGTGGTGAATCACCTTGTTCCATTGATGATGA |
| | | GCAATTGATTGGTTCACCATCTCTGACACCAATCACTATG |
| | | GTGGCAAAACTAATCGATGGATATCTTGCTGAGGTTGCCC |
| | | CAGATGTTAACTTGAAGCTTCCCAAATTTCAGACTCTTGC |
| | | TGCTTCTGTTCCTGAATATGCAAGGCCCTTGGATGATGGC |
| | | CTTTATCGTGCAATTGACATTTATCTCAAGTCACATCCAT |
| | | GGTTGGGAGAATCTGATAGGGAACAACTCTGCAGGCTCAT |
| | | GGATTGCCAGAAACTCTCCTTGGAAGCTTGTACACATGCT |
| | | GCACAGAATGAACGGCTCCCTTTAAGGATTATAGTACAAG |
| | | TACTCTTCTTTGAACAGCTGCAGTTGAGAACATCCATTGC |
| | | AGGATGCTTCCTAGTGTCTGAAAACCTTGATGAGGGGTCA |
| | | AGGCAGTTAAGAAGTGGCACGGTGGGACCAAATGAGGGGG |
| | | GCTGGGCTACTGCTGTGAGAGAAAACCAGGTGTTGAAGGT |
| | | GGGAATGGATAGTATGAGGGTGCGCGTGTCAGAGCTCGAG |
| | | AAGGAGTGCTCAACCATGAGGCAGGAGATTGAGAAGTTGG |
| | | GTCGATCAAAGGGATCAAGCACTTGGGGAAATGTATCCAA |
| | | GAAGTTTGGTTTCAAGATGAAATCTCAAATGTGCAGTGCT |
| | | CAGGAGGGATCTGTTAGTAACCAGAACAAGATAAATAGTA |
| | | AGGCTATAAAGGATGACAAGGCAAAAGAAAACTATCGAAA |
| | | GCACTAG |
| SEQ | Solyc02g067030.4 | TTTTTTTATGGACCACTTTGGCCCAAAAAGGTACACTACT |
| ID | | GGTTAGCTGTAGCAGATTCTGTGGAAGGAAAACATCAAAG |
| NO: | | GAAAATGGACGGAACAGCAGTGCAGGGCACCAGCAGTGTT |
| 153 | | GACTCATTTTTACGGAACTATAAACTCGGGAAAACACTTG |
| | | GCATTGGATCGTTCGGCAAAGTTAAAATAGCTGAACATAC |
| | | GTTAACAGGGCACAAAGTTGCTGTCAAGATTCTTAATCGT |
| | | CGAAAAATCAGGAATATGGACATGGAGGAGAAAGTCCGTA |
| | | GAGAAATCAAAATATTGAGATTGTTCATGCATCCTCATAT |
| | | TATACGGCTTTATGAGGTCATAGAGACACCATCAGATATA |
| | | TATGTTGTGATGGAGTATGTGAAATCTGGCGAGTTATTTG |
| | | ATTACATTGTTGAGAAGGGCAGATTGCAGGAGGATGAAGC |
| | | TCGTAACTTTTTTCAGCAGATAATTTCTGGTGTGGAGTAC |
| | | TGCCATAGAAACATGGTGGTTCATAGAGACCTTAAGCCTG |
| | | AAAACCTCCTTCTGGACTCCAAATGGAATGTGAAGATCGC |
| | | AGATTTTGGTTTGAGCAATATCATGCGCGATGGTCATTTT |
| | | CTGAAGACAAGTTGCGGAAGCCCAAACTATGCTGCCCCAG |
| | | AGGTTATATCAGGTAAATTGTATGCTGGCCCTGAGGTAGA |
| | | TGTATGGAGCTGTGGTGTTATTCTTTATGCTCTTCTCTGT |
| | | GGCACCCTTCCGTTTGACGATGAAAACATACCCAATCTTT |
| | | TTAAGAAAATAAAGGGTGGAATATATACTCTGCCCAGCCA |
| | | TTTATCAGCTGGTGCGAGGGATTTGATTCCGAGGATGCTT |
| | | ATAGTCGACCCAATGAAGCGAATGACTATTCCTGAGATTC |
| | | GCCTGCACCCTTGGTTCCAAGCTCATTTGCCACGCTATTT |
| | | GGCCGTGCCTCCACCAGATACAACCCAACAAGCAAAGAAG |
| | | ATCGATGAAGAGATTCTTCAAGAGGTGGTTAAGATGGGAT |
| | | TTGACAGGAACAACCTTACTGAGTCTCTTCGCAATAGAGT |
| | | TCAAAATGAGGGCACTGTTGCATACTATCTGCTCCTGGAC |
| | | AATCGCCATCGTGTTTCCACTGGCTATCTTGGAGCTGAAT |
| | | TTCAGGAGTCCATGGAATATGGTTACAACCGGATCAATTC |
| | | TAATGAAACCGCTGCTTCCCCTGTTGGTCAACGTTTCCCA |
| | | GGAATAATGGATTATCAGCAAGCTGGTGCAAGACAGTTCC |
| | | CCATTGAAAGAAAATGGGCTCTTGGCCTCCAGTCTCGAGC |
| | | GCATCCACGTGAAATAATGACTGAAGTTTTGAAAGCTCTG |
| | | CAAGAACTGAATGTATGTTGGAAAAAGATTGGTCAGTATA |
| | | ACATGAAATGTCGATGGGTTCCTAGCTTACCTGGTCATCA |
| | | TGAAGGCATGGGTGTTAATTCCATGCATGGGAATCAGTTC |
| | | TTTGGAGATGATTCATCCATCATTGAGAATGATGGGGCCA |
| | | CAAAGTTAACAAATGTGGTCAAGTTTGAAGTTCAGCTTTA |
| | | CAAAACCAGGGAGGAGAAGTACTTGCTTGACCTTCAGAGA |
| | | CTTCAGGGTCCACAATTCCTCTTCCTGGATCTCTGTGCTG |
| | | CTTTTCTTGCTCAGCTTCGAGTACTTTAA |
| SEQ | Solyc02g067860.4 | ATGTCGTCGAACATAATTCACGACGCCGATGGATCGGCTT |
| ID | | CGCGAATCGTATTGAAGGAGCATACTGGATCAACTGCAGA |
| NO: | | GGTGCTTTTTCAGGGTGGTCAAGTTGTTTCTTGGAAGAAT |
| 154 | | GAACGAAGAGAAGAACTGCTGTTCAGGAGTAATAAGACCC |
| | | TGTGGAAACCTCCTAAAGCCTTCAGAGGTGGAATACCTAT |
| | | CAGCTTCCCACAGTTTGGAACTTTTGGTTCTTTAGAGAGA |
| | | CATGGTTTTGCAAGAAATAGAGTGTGGGAATTGGACGATT |
| | | CTCCTTCTCCTCTACCTGCAGCAAACAATCAGTCAACTGT |
| | | TGATCTCATTTTGAAATCTACCGAAGAGGATCTGAAGACC |
| | | TGGCCGCATAGGTTTGAGGTGCGGCTACGCATTTCTCTCA |
| | | GTGCTGGCAAGCTGACTTTGATTCCTCGTGTCAGGAATAT |
| | | AGATAACAAGCCATTCTCATTCACATTCTCTCTGCGTAAT |
| | | TATTTATCTGTGTCTGATATCAGTGAAGTGCGGGTTGAGG |
| | | GCTTGGAGACACTTGACTACTTCGATAATTTGCAGCGGGG |
| | | AGAAAGATACACAGAGCAGGCAGATGCAATTACATTTGAT |
| | | AGTGAGACGGATAGAGTATATCTTAGCACACCAACAAAAA |
| | | CTGCCATAATAGATCACGAGAAGAAGAGAACTTATGTCCT |
| | | TCAGAAAGAAGGAATGGCAGATGCTGTTGTTTGGAATCCT |
| | | TGGGACAAAAAGGCCAAGTCTCTCCCAGATTTGGGTGATC |
| | | AAGATTACAAGAAAATGTTGTGTGTGGATTCTGCTGCTGT |
| | | TGAAACTCCGATTATCTTAAAACCTTCTGAAGAGTGGAGG |
| | | GGGCGGCAAGAGCTCTCTACGGTATCTTCAAGTTATTGCA |
| | | GTGGACAATTGGACCCTAGGAAAGTTATTTATGGCTAA |
| SEQ | Solyc02g068130.4 | ATGGAGATGCAAAGCGAATTCGATCGTCTCCTTTTCTTCG |
| ID | | AACACGCTCGGAAGAGTGCTGAAACCGCCTATGCTCAGAA |
| NO: | | TCCCCTAGATGCTGATAACTTGACGAGGTGGGGAGGTGCA |
| 155 | | TTGCTGGAATTATCACAGTTTCAGCCCGTAGCTGAATCGA |
| | | AACAAATGATTTCTGATGCAACATCAAAGTTGGAGGAGGC |
| | | ATTGACAGTGAACCCTGAAAAGCATGATGCGCTTTGGTGT |
| | | TTGGGAAATGCTCATACATCACATGCTTTTTTAACTCCTG |
| | | ATATGGATGAGGCTAAGGTTTACTTTGAAAAAGCAACTCA |
| | | GTGCTTTCAACAGGCATTTGATGCGGATCCCTCAAATGAC |
| | | CTTTACCGCAAATCTCTGGAAGTTACTGCTAAGGCTCCAG |
| | | AATTGCACATGGAAATCCACAGACATGGTCCCATGCAACA |
| | | ACCTATGGCAGCAGAACCTTCAACATCGACAAGCACAAAG |
| | | AGTTCAAAAAAGAAGAAGAGCAGTGATCTCAAGTATGACA |
| | | TTTTTGGGTGGGTCATACTTGCCGTTGGCATTGTTGCATG |
| | | GGTGGGATTTGCAAAATCTAATATGCCTCCACCACCACCA |
| | | CCGCATCCACAGTGA |
| SEQ | Solyc02g069090.4 | CTTGTATCTATAAATATATTTTCTGGGGTTCCTCCATTTT |
| ID | | TAAGCCACCATTACAGAACCCAAGAAAAAGGGCCCAAAAA |
| NO: | | TATGAAGCACATAGCCACTTTTTTGCTTTTGGTTTCTGTC |
| 156 | | TCCACCCTTGTTTTGCAGGTTGTTGCAGAAAATCCAATAT |
| | | CCCAAGCTAAAGCGGAATCTGCAATCCTTCAGGACTCAAT |
| | | CGTCAAACAGGTTAATGAAAATGAAAAAGCTGGATGGAGA |
| | | GCTGCACTAAACCCTCAATTCTCGAATTTCACGGTTTCCC |
| | | AATTTAAGCGCCTTCTTGGAGTTAAGCCCACAAGAAAGGG |
| | | TGATTTGAAGGGCATCCCTATTTTAACTCATCCAAAACTT |
| | | TTGAAGTTACCCCAAGAGTTTGATGCACGAGTGGCTTGGC |
| | | CTCAGTGTAGCACTATTGGGAGAATTCTTGATCAGGGACA |
| | | TTGTGGTTCTTGTTGGGCTTTTGGTGCTGCTGAGTCGTTG |
| | | TCTGATCGTTTCTGTATTCATTATGGATTGAATATCTCTC |
| | | TGTCAGCAAATGATATCATAGCATGCTGTGGCTATTTATG |
| | | CGGGGATGGTTGTGATGGTGGATATCCGTTAGAAGCTTGG |
| | | AAATACTTTGTTCGCAAGGGTGTGGTCACTGAAGAGTGTG |
| | | ACCCTTACTTTGATAATAAGGGATGTTCTCACCCTGGATG |
| | | TGAACCTGGATATCCCACTCCACAGTGCAAGAGGAAGTGT |
| | | GTTAAAGAGAATCTACTCTGGAGTAAATCCAAGCATTTTG |
| | | GTATTAATGCATACCTGATTAACTCCGACCCATACAGTAT |
| | | CATGACAGAAGTTTACAAGAATGGACCAGTTGAGGTTTCT |
| | | TTTACTGTTTACGAGGATTTCGCTCACTACAAGTCTGGTG |
| | | TTTACAAGCACATAAATGGTGAGGAAATGGGAGGCCATGC |
| | | TGTTAAGCTCATTGGATGGGGAACCTCTGAGGATGGAGAG |
| | | GACTATTGGCTCCTTGCCAATCAATGGAACAGAGGATGGG |
| | | GTGATGATGGTTACTTCAAGATCAGAAGAGGAACAAACGA |
| | | GTGTGGCATTGAAGAAGAAGTGGTTGCAGGAATGCCTTCA |
| | | GCCAAAAACCTGAACGTGGAACTTGATGTCTCTGATGCTT |
| | | TACTTGACGCCTCAATGTGA |
| SEQ | Solyc02g069100.4 | AATTGGAAAGCTTTAAAGAAAGAAAAAAAAAACATGGCCT |
| ID | | TGACTTTGAAGTCATTAATAACTCCTTTGCTTTTTGGTGC |
| NO: | | TTTCTTTATCCTGATCTTACAGCAGGTTGCTGCAGAGAAG |
| 157 | | CCAATAACTGAAGCAAAACTTGAATCTGCAATCCTTCAAG |
| | | ACTCGATCGTCAAACAAGTCAATGAGAATGCCGAAGCTGG |
| | | ATGGAAAGCTGCATTCAACCCTCAATTATCGAATTTCACG |
| | | GTTTCCCAATTTAAGCGCCTTCTTGGAGTTAAGCCTGCAA |
| | | GAGAAGGTGATTTGGAGGGAATTCCTGTTCTAACTCATCC |
| | | AAAACTTAAGGAGTTACCAAAAGAGTTTGATGCACGAAAG |
| | | GCTTGGCCTCAATGCAGCACAATTGGAAGAATTCTAGGTC |
| | | AGGACATTGCGGTTCTTGTTGGGCTTTTGGTGCTGTTGAG |
| | | TCGCTGTCTGATCGTTTCTGTATCCATTATAATTTGTATC |
| | | TCTCTGTCTGTAAATGATCTGTTAGCGTGCTGTGGCTTTT |
| | | TATGCGGGAGTGGTTGTGATGGTGGATATCCTATAGCAGC |
| | | ATGGCGATACTTTAAACGTAGGGGAGTGGTCACAGAAGAG |
| | | TGTGATCCTTACTTCGACACTACTGGATGTTCCCACCCTG |
| | | GTTGCGAACCACTATACCCCACTCCAAAATGTCATAGGAA |
| | | GTGTGTCAAGGGGAATGTGTTATGGAGGAAATCAAAGCAT |
| | | TACGGTGTAAATGCATACAGGGTCAGCCATGATCCCCAGA |
| | | GTATCATGGCGGAAGTATACAAAAACGGACCAGTTGAGGT |
| | | CTCTTTTACTGTCTACGAGGATTTTGCTCACTACAAATCA |
| | | GGAGTTTACAAGCATGTAACAGGGGGAAACATGGGAGGAC |
| | | ACGCTGTTAAGCTTATCGGATGGGGAACTAGCGAGCAAGG |
| | | AGAGGACTATTGGCTCATTGCTAATTCTTGGAATAGAGGC |
| | | TGGGGTGAAGATGGATACTTCAAGATTAGAAGAGGAACAA |
| | | ATGAGTGTGGCATTGAACATAGTGTGGTTGCAGGATTGCC |
| | | TTCAGCAAGAAATTTGAATGTGGAACTTGGTGATGCTGTC |
| | | CTTGATGCCTCAATGTAA |
| SEQ | Solyc02g080630.4 | ATGGGGTTTTCCGTCTTTTTATTCACCTTTCTCTTACAAC |
| ID | | ACCTTCTAATATTCAACCACGTGTTCTATCTCTGTAGTAC |
| NO: | | CGACCGTTGTTCAAATCGATTCTTCACTTTACCACGACTG |
| 158 | | ATCTCACTCTCTCTCAATCTGAACATGGCGGAAGCAAGTG |
| | | CACCAGCTGTACCAAGTACTGAGTTGTTGGAGTGGCCAAA |
| | | GAAAGATAAGCGCAGAATGTTGCATGCTGTATATCGTGTC |
| | | GGTGACCTTGAACGTACCATCAAGTTTTACACAGAATGTT |
| | | TTGGGATGAAATTGTTGAGGCAGAGAGATATTCCAGAGGA |
| | | GAAGTATTCGAATGCTTTTCTTGGTTTTGGCCCTGAAGAG |
| | | TCTCACTTTGTGGTTGAGTTGACATATAATTATGGAGTTG |
| | | ATAAGTATGACATCGGAACCGGCTTTGGGCATTTTGCTAT |
| | | TGCCACACCAGATGTTTACAAACTGGTTGAGGAGATAAAG |
| | | GCCAAGGGTGGAACTGTCACAAGGGAGCCTGGTCCTGTCA |
| | | AGGGTGGATCTAGTGTTATTGCTTTTGTCAAAGATCCTGA |
| | | TGGCTACCTATTTGAAATCATCCAGAGAGAGTCTACTCCT |
| | | GAACCACTTTGCCAAGTGATGCTTCGTGTGGGGGATCTTG |
| | | AACGTGCGATCAAATTCTACGAAAAGGCACTTGGGATGCA |
| | | AGTGGTGAAGAAAGTTGATAGACCAGAGCATAAGTATACC |
| | | ATTGCTATGATGGGATATGCTCCAGAGAAAGAGACGACTG |
| | | TTATTGAGTTGACATACAACTATGGTGTGACTGAATACAC |
| | | AAAGGGAAATGCATACGCACAGATTGCAATAGGCACTGAT |
| | | GATGTCTACAAAAGCGCGAAGGTTGTCAATCTTGCTATCC |
| | | AAGAGCTCGGAGGAAAGATAACTAGACAGCCAGGATCAAT |
| | | TCCTGGACTTAACACCAAGATAACTTCATTTCTGGATCCA |
| | | GATGGCTGGAAAACAGTGCTGGTTGACAATAAGGATTTTC |
| | | TGAAGGAGCTAGAATCGTCCAAGTAA |
| SEQ | Solyc02g081210.3 | ATGCTACTACTGTCACTTTTAGTAGCATTCGCACTCTTTT |
| ID | | CTCCAACCCCATTTGTCCATTCTCACTACAACTGGTCACC |
| NO: | | TTCTTCTTCCTCCACCTCCAATTTTCAGTCCGAGTGGCGT |
| 159 | | CCTGCACGCGCCACCTATTACGCCGCCGCCGACCCACGTG |
| | | ACGTTGTGGGTGGCGCGTGCGGATATGGGGATTTGGAGAG |
| | | AAATGGGTATGGAAAAGCTACAGCTGGGTTAAGTACTGTG |
| | | CTTTTTGACAAGGGTCAGATCTGTGGTGCTTGTTATGAAG |
| | | TGAGGTGTGTGGAGGATCTGCGGTGGTGCATTCCGGGTAC |
| | | TTCTATAATTGTTACAGCCACTAATTTTTGTGCACCAAAT |
| | | TATGGGTTTGATCTAGATGGTGGTGGTCACTGTAATCCAC |
| | | CTAATGCCCATTTTGTGCTACCCATTGAGGCTTTTGAGAA |
| | | GATTGCCATTTGGAAAGCTTCTAATATGCCCATTCAATAC |
| | | CGCAGGATAAAGTGCAGAAAGGAAGGTGGAGTTCGATTCA |
| | | CTCTTGGTGGCTCTGGTATATTCTTGTCAGTTCTAATCAG |
| | | TAATGTTGCAGGTTCGGGTGATATAGTGGCAGCAAAAGTT |
| | | AAGGGTTCAAGAACAGGCTGGCTTCCTATGGGTAGGAACT |
| | | GGGGCCAAAACTGGCACATTAATGCTGACTTAAAGAATCA |
| | | GCCGCTTTCTTTTGAGATGACTAGTGGTGATGGGGCCACC |
| | | TTAACATCTTACAATGTAGCTCCCAAGAATTGGAATTTTG |
| | | GTCAGACCTTTGAAGGCAAGCAGTTTGGGTCTTAG |
| SEQ | Solyc02g084720.3 | ATGGAGGTTAACTCACTTCAGAAGTGGGTTCTGCTATGGT |
| ID | | GTATAGTATTGTTTATTAGCTCTGGGTTGGTTCACTGTGA |
| NO: | | TGTTACATATGATAGGAAAGCTATTGTGATTAATGGGCAA |
| 160 | | AGAAGATTACTTTTCTCTGGTTCTATACATTACCCTAGAA |
| | | GTACCCCTGAGATGTGGGAAGATCTGATAAACAAGGCAAA |
| | | AGAAGGGGGTTTGGATGTGGTTGAGACCTATGTCTTTTGG |
| | | AATGTTCATGAGCCTTCTCCTGGCAATTACAATTTTGAAG |
| | | GAAGATATGACCTGGTGAGGTTTGTAAAAACGATTCAGAA |
| | | AGCAGGGCTGTATGCTCATCTTCGAATTGGCCCTTACGTT |
| | | TGTGCAGAGTGGAATTTTGGAGGGTTTCCAGTATGGCTGA |
| | | AGTATGTACCTGGCATTAGCTTCAGAGCTGATAATGAACC |
| | | TTTCAAGAACGCAATGAAAGGGTATGCTGAGAAAATTGTT |
| | | AACTTGATGAAGAGTCATAATCTTTTCGAGTCTCAGGGTG |
| | | GTCCAATCATACTCTCACAGATTGAGAATGAGTATGGGCC |
| | | TCAAGCCAAGGTACTTGGAGCACCGGGACATCAGTATTCA |
| | | ACATGGGCTGCAAATATGGCAGTTGGATTAGACACAGGCG |
| | | TCCCATGGGTGATGTGCAAGGAAGAAGATGCACCAGATCC |
| | | TGTGATCAACACATGCAATGGTTTCTACTGTGATAATTTC |
| | | TTCCCAAACAAACCATACAAACCTGCAATTTGGACTGAAG |
| | | CTTGGAGTGGATGGTTCTCGGAATTTGGCGGTCCCCTTCA |
| | | TCAGAGACCAGTTCAGGATTTGGCATTTGCTGTTGCCCAA |
| | | TTTATACAAAGAGGAGGATCTTTTGTTAACTATTACATGT |
| | | ACCATGGGGGCACGAACTTTGGACGCACTGCGGGTGGGCC |
| | | ATTCATCACTACCAGCTATGATTATGATGCTCCAATTGAT |
| | | GAGTATGGTTTGATCAGACAGCCAAAATATGGTCATCTCA |
| | | AGGAGCTTCATAGAGCTGTCAAGATGTGTGAGAAATCCAT |
| | | AGTTTCTGCAGATCCAGCAATCACATCCTTGGGAAACCTT |
| | | CAACAAGCATATGTGTACTCTTCAGAGACAGGGGGTTGTG |
| | | CAGCTTTCTTGTCAAACAATGACTGGAAATCTGCTGCTAG |
| | | AGTAATGTTCAATAATATGCACTATAATTTGCCTCCTTGG |
| | | TCCATTAGCATCCTTCCTGATTGCAGAAATGTAGTCTTCA |
| | | ACACGGCCAAAGTTGGAGTTCAAACATCAAAGATGGAAAT |
| | | GCTACCGACTAACTCAGAGATGCTATCTTGGGAGACTTAC |
| | | AGTGAAGATATATCTGCATTAGATGACAGCTCATCAATTA |
| | | GGTCTTTTGGTCTCTTGGAGCAAATAAATGTAACCAGAGA |
| | | TACAAGTGATTATTTGTGGTACATAACTAGTGTTGATATT |
| | | GGTTCAACTGAGTCCTTCTTGCATGGTGGAGAGCTCCCTA |
| | | CTCTTATAGTTGAGACAACAGGCCATGCTATGCATGTGTT |
| | | TATTAATGGTCAGCTCTCAGGTTCTGCATTTGGGACAAGG |
| | | AAGAATAGGAGATTTGTATTTAAAGGAAAAGTCAATCTTC |
| | | GTGCTGGCTCAAACAGAATTGCACTGCTTAGCGTGGCTGT |
| | | CGGGCTGCCGAACATTGGTGGACATTTTGAAACATGGAGC |
| | | ACTGGCGTACTAGGTCCTGTTGCAATTCAAGGACTTGACC |
| | | ATGGAAAATGGGACTTGTCCTGGGCAAAGTGGACATATCA |
| | | GGTTGGGCTTAAAGGGGAGGCCATGAATCTTGTTTCGACA |
| | | AATGGTATTTCTGCCGTTGACTGGATGCAAGGTTCATTAA |
| | | TTGCACAGAAACAACAACCCTTGACATGGCATAAGGCTTA |
| | | CTTCAATACACCCGAAGGAGATGAGCCATTGGCTTTGGAC |
| | | ATGAGCAGTATGGGTAAAGGTCAAGTATGGATTAATGGCC |
| | | AGAGTATTGGTAGATATTGGACTGCATATGCTACTGGAGA |
| | | TTGCAATGGGTGCCAATATTCTGGAGTTTTTCGGCCTCCA |
| | | AAATGTCAACTGGGATGTGGGGAACCAACTCAAAAATGGT |
| | | ACCATGTTCCTCGGTCTTGGTTAAAACCCACTCAAAATCT |
| | | GTTGGTGCTTTTTGAAGAACTTGGCGGAGATCCCACAAGA |
| | | ATTTCTCTCGTGAAAAGATCAGTCACTAACGTATGTTCGA |
| | | ATGTAGCTGAATATCATCCAAACATTAAGAACTGGCAAAT |
| | | TGAGAACTACGGCAAAACCGAAGAGTTTCACCTACCCAAA |
| | | GTTCGCATTCACTGTGCTCCTGGTCAATCTATTTCCTCCA |
| | | TTAAATTTGCAAGCTTCGGAACTCCTCTCGGAACCTGTGG |
| | | GAGCTTCAAGCAAGGAACTTGCCATGCTCCCGACTCACAC |
| | | GCCGTTGTAGAAAAGAAATGTCTAGGACGCCAAACATGTG |
| | | CTGTGACAATATCAAATAGCAACTTTGGAGAAGATCCCTG |
| | | CCCCAACGTACTGAAGCGGTTGTCTGTAGAAGCACATTGT |
| | | ACACCTACTCAGAATTGA |
| SEQ | Solyc02g087530.2 | ATGTCGACACTTATTTGTTTAGCAAGTCCGTCGACACTGG |
| ID | | TGCTAAGTATCTCAAAGTTGAAAAACCCAAAAACAGCAAG |
| NO: | | TCTTTCTGTGTTATGTTGGACGAAGGAGAAGAAATCATTG |
| 161 | | ATGAATTCGAAGGAGCTACATTCTGCTGGCGCTCTCACAT |
| | | TGAAACGTTCGATGATAATTCTCAGGGGAGTAAAAAAAAA |
| | | AACCCAGCCTAGTCAGAAGAAAAGTTACATAATTACATTC |
| | | AATAAGCAACATCGAGAGATTGTCATCGGAAAATACTTGA |
| | | AACATGTGATTGAAGAAGGCAGGGCAATTGAGTTCAAGAA |
| | | TAAGAAGCAGAAGATTTATTGTAACGATAGAATGCCTTCT |
| | | TGGTGGGCGTGTTACAACTCAATGTGGAAGCATATCAATT |
| | | TTGAGCACCCTGCAACTTTTGATACTCTGGCTATGGATCC |
| | | TAAGAAGAAAGCGGAAATAATCGACGATCTCATTGCTCTC |
| | | ACCAACGGGAAGGATTATTATTCCAAAGTTGGAAAGGCGT |
| | | GGAAGCGCGGTTATCTTCTTTATGGCCCACCAGGAACTGG |
| | | AAAATCAACTATGATTGCAGCAATTGCAAATTACTTGAAC |
| | | TATGATATTTATGATCTTGAGCTAACTTCTGTAAAGGATA |
| | | ACTTAGAACTTAAGAAGTTGCTCATGGAAATAACAAGCAA |
| | | GTCTATTATTGTCATTGAAGACATTGATTGTTCCGTTGAT |
| | | CTCACTGGAAAGAGAAAGAAGGCAAAAAAGAATAAGAAGA |
| | | AGGATGACTCAGATTCCGACTCAGATTCCGATTCTGATTC |
| | | AGACTCAGACTCAGACTATGAAGGCAAAAAAAAACTCACA |
| | | CTATCAGGGTTGTTGAATTTCATTGATGGCATTTGGTCAG |
| | | CTTGTGGTGAAGAGAGAATTATTATATTTACCACTAATCA |
| | | TGTGGACAAACTTGATCCAGCTTTGATACGTAGAGGACGC |
| | | ATGGATATGCACATTGAGATGTCGTATTGCAGATACGAAG |
| | | CATTCAAAGTGTTGGCTAAGAATTACTTGGGCATTGATAC |
| | | ACATCCTTTGTTTCAAGAGATTCAACCTTTACTAGAGGAA |
| | | GTAAACTTGAGTCCTTGTGATGTCGCTGAGACCTTGATAC |
| | | TAAAGAATACTTCGGGAGGGATTGAGATTTGCTTGAATAA |
| | | CTTGATTCTGGCTTTGAAAGATTCCAAGGAGAAGGCAATA |
| | | CAAGAGGCCAGGGAGAAGGAAAAACAAGATTCGGAAGAAA |
| | | AACCAAAGAAGAATAAAAAGTTAAAAAAAATTTATGGCCG |
| | | TTTTAAAAAGCTTTTCAGATGA |
| SEQ | Solyc02g087770.3 | ATGTCTCTCAAGATTAATCTGTTGATCTGTTTCTTCATTC |
| ID | | TTCATCTATGGGCTGCTACAGGACACAAGATAGGAATTTA |
| NO: | | TGAGATCAAGAAAGGAGATTTCTCTGTTAAGGTCACTAAT |
| 162 | | TATGGTGCCAGAATCATCTCTGTTCTTCTTCCTGATAAGA |
| | | ATGGAAAAATAGGTGATATTGTTCTTGGATATGACACCAT |
| | | TGGGGAATACGTGAACGATACGAGTTCCTTTGGAGCTATT |
| | | GTTGGAAGAGTTGTTAACCGGATTGCTGGTGCTCGATTTA |
| | | CTCTAAATGGAACCCTTTATAAGCTTATCGCCAATGATGG |
| | | CAAGAACACAATACACGGTGGCCCTAAAGGATTTAGCCAT |
| | | GTTGTTTGGAAGGTGAGCAAGTACAAGAAAGATGGTCCTC |
| | | ATCCTCACATTACTTTAACCTACCACAGTACTGATGGTGA |
| | | ACAAGGATTTCCTGGTGATCTTCTTGCCTCTGTTACCTAT |
| | | GCATTGGAAGACCCTTACAAGCTTAGTGTGGTAATGAAAG |
| | | CAAAAGCTCTAAACAAGGCCACTCCAGTTAACTTGGCTCA |
| | | ACATACTTATTGGAACCTTGGTGGACACAACAGTGGCGAT |
| | | GTTTTGTCCAACGATATTCAAATCTTTGCTTCACACATCA |
| | | CCCCATTAGATCAACATTTCATTCCAACAGGAAAAATCGC |
| | | CCCTGTCAAGAACACGCCCTATGATTTCCTTAAACCCCGT |
| | | AAAATTGGGAGCCAGTTCAAGAAACTCCAAAATGGATATG |
| | | ACATAAACTATCCACTAGACAGCACTGAAAAACTGAAGCC |
| | | CACGGCAACAGTTCACGATAATAAGTCAGGAAGAGTGATG |
| | | AGTTTACAAACGACTGCACCTGCTGTCCAATTATACACTG |
| | | CAAACACCCTTGTAAAGAAAGGGAAAGGTGGATTTGTCTA |
| | | TCAGCCTTATTCAGCCGTGTGTTTGGAGACTCAAGGATTC |
| | | CCTGATGCTGTGAATCACCCAAATTTCCCATCAACAATTG |
| | | TGACTCCGGAACATCCCTACTTTCACAAAATGCTGTTTAC |
| | | GTTCTCAATTAAGAAATATTAG |
| SEQ | Solyc02g089070.3 | ATGACGACTGTGGTTCCTACTTCTGAGGAAGATCCCGTCC |
| ID | | TCTCCGTCGTCCGGTTTACGGCAGAGATGTCATGGGCGGA |
| NO: | | CGCCGGCGCCGAGACTGCAGAGCCTGAAGTTAATAGATTG |
| 163 | | TGTGTCGAGGCCCAGAAATGCATGATTGAAGGAAGGTGGT |
| | | TGGATTTGGTTTCGCTTATGCTCGCTTCTGCAGATATAAT |
| | | ATTTTCTAAAGCTTCTGAAAAAGATCTTGAGTGCGTTTTC |
| | | ACAATAATGTGCAACCTTGTCAAGAAGCCTGAGAGCTTGG |
| | | ACCAAGTACATGAGATGGCAGAGCTCATATCCGCTAAAAT |
| | | CATTCAACAACCCAATGATAAACCCGCATTGCGCTTGAAA |
| | | ATCTTGTTCAATCTCTACAATCTGTTGGAGAACCCATACA |
| | | GCCGTTTCTGCGTTTACTTGAAGTCCCTCAAGTTGGCCAC |
| | | TGCCGGGAAGGTTACTGAACACATTTTGTCGTCCATCAAA |
| | | ATGATGGATAACTTCTTGAAGGAGTGGAATCTTGGAGTCA |
| | | AAGATCAGAGAGAGCTGTTTCTTGCAATCTCCAACATTTT |
| | | AAAGGAAAGCAAAGGCTCTACAAAAGATTCTTTCATGTTT |
| | | CTTACAAAGTATCTGGAAACCTTTTCGTCTGAAGATGCTT |
| | | CTAATATGACTGAGGCTAAGGAAGAAGCTGCCAGTGCAAT |
| | | TGTCGATTTTGTCAGATCACCTGATATGTTTAAGTGTGAC |
| | | TTGCTAGATATGCCGGCTATAGTCCAGTTGGAGAAAGATG |
| | | GTCAATATGGTCCAGTATATCAACTTCTGAAGATCTTTCT |
| | | CACTCAGAGGCTGGATGCTTACTTGGAATTTGAGGCAGCA |
| | | AATTCAGCATTATTAAAAACCTATGGACTTGTCCATGATG |
| | | ACTGTATATCAAAGATGAGGTTGATGTCGTTGGTAGATCT |
| | | TGGCTTGAATGAATCTAGTCAAATTCCTTATTCTGTCATC |
| | | AAACAGGCATTGCAGATTGATGACATTGAGGTAGAATCGT |
| | | GGGTTGTTAAGGCAATTACAGCTAAGTTACTTGATTGCAA |
| | | GATTGATCAAATGAATCAAGTAGTAATAGTGAGCCGATGT |
| | | ACGGAGCGTGTGTTTGGTGTGCATCAGTGGCAAGATCTCC |
| | | GCACGAAACTAGTGACTTGGAGGGGCAACATTGCTGGTGT |
| | | TATCAGCACAGTTCAAGCCAATAAGATTACAGAAGATAGC |
| | | ACACAGGCAGTGCAAGGTTTAGCAATCCGCTGA |
| SEQ | Solyc02g092750.3 | ATGTTAAGCTCCTGTTTTGGGAGCTGCTTTGGTGGGGACG |
| ID | | ATGGTGATGATCTTTTATGGCATATGGACTTGAAGCCACA |
| NO: | | TGCCACAGGTGAATATTCTGTTGCTGTAGTTCAGGCCAAT |
| 164 | | TCTTTGCTTGAAGATCAAGGTCAAGTTTTTACAACCCCTA |
| | | GTGCTACTTATGTGGGTGTTTATGATGGTCATGGTGGCCC |
| | | GCAAGCTTCTCGCTTCATCAACAACAATCTCTTCCCTCAA |
| | | CTACAAAAGCTGGCTTTGGAAGAAGGAGGATTGTCTGAGG |
| | | ACGTGATCAGGAAAGCATTTGATGCTATTGAGGAGGGATT |
| | | CTTGCGTTTGGTGAAACAGTCTTGGCTGGATCAACCTCAG |
| | | ATTGCATCTGCAGGGTCTTGTTGTCTATTTGGAGCAATCT |
| | | CAAAAGATGAATTGTATGTGGCAAATCTTGGGGACTCCAG |
| | | GGCAGTGCTTGGCAGGAGAGCAGAGACAGGCCAGACGGGT |
| | | GATTCAGCAGTAGTAGTGGCAGAACGTTTGTCGATAGATC |
| | | ATAATGTTGGGGTTGAGGAGGTTAGGAAGGAGGTAGAGGC |
| | | ACTGCATCCAGATGATTCTCATGTTGTGGTCTACGTTCGA |
| | | GGGGTTTGGAGAATTAAGGGTATAATTCAGGTTTCTAGAT |
| | | CAATTGGTGATGTCTATCTGAAGAAACCTGAGTTTAATAG |
| | | AGATCCTATGTTCATACAATATGGATATCCTATTCCTTTA |
| | | AAAAGAGCTGTAATGTCAGCAGAGCCCTCCATATTGATCC |
| | | GAAAGATAAGACCAGAAGACTTATTTTTGATATTTGCATC |
| | | GGATGGCCTATGGGATCAACTAACCGATGACGAAGCTAAG |
| | | GATATTGTTTTGAAGAATCCTAGAATTGGAATAGCAAAAC |
| | | GATTAGTGAGAGCTGCCCTTGAGGGTGCTGCAGAGAAGAA |
| | | AGAAATGAGATATGAGGACATCAAACGACTAGAGAAGGGG |
| | | GAAAGGCGGCACGTTCATGATGATATCACAGTTATTGTGA |
| | | TATATTTGGATCACCCTGGAAGGTCCTCAAATTCTGGAAT |
| | | TAACATTGTTAAGGGCACGAATGTCCCTCTTGACATATAC |
| | | TCCTTGAACTCGGGTCATTAA |
| SEQ | Solyc03g007240.3 | ATGGATGGGAAGGAGAATAATGGTGTTAATGGTTCAAACA |
| ID | | AAGTTGGTATTCCTAAATGTTGCTTGAAGGCTAGGGTATT |
| NO: | | TGATCCTGAACTTGAAGCCAATTGTCACTCTACTGTTGTT |
| 165 | | TCTGGATGGTTCTCTCAACCTCCTACATCATTTGATGGTA |
| | | AAGAGAAGGTACTGTACTTCAACAATCCAATGTGGCCAGG |
| | | AGAAGCACATTCCCTGAAAGTAGAAAAGGTTTTGTTCAAG |
| | | GGAAAGTCAGAATTCCAGGAGGTTTTGGTTTTTGAGTCTG |
| | | CATCCTATGGAAAAGTTCTAGTGCTAGATGGCATTGTTCA |
| | | GTTGACTGAGAAAGATGAATGTGCCTATCAGGAGATGATA |
| | | GCCCATCTTCCTCTTTGTTCAATCAAATCTCCTAAGAAGG |
| | | TTCTGGTTGTTGGGGGTGGTGATGGTGGAGTTCTAAGGGA |
| | | AATTTCTCGACATAGCTCTGTGGAGCTGATTGATATTTGT |
| | | GAGATTGATAAAATGGTTATAGATGTCAGTAAAAAGTATT |
| | | TTCCAGATTTGGCTATTGGCTTTGAGGATCCTCGTGTCAA |
| | | TCTTCATGTTGGTGATGCTGTTGAATTTCTGAAGAACACA |
| | | CCTGAAGGGAAGTATGATGCTATCATAGTTGATTCATCAG |
| | | ATCCTGTAGGTCCTGCAATAGAGCTTGTTGAAGGACCATT |
| | | CTTTGCAAAGATAGCCAGGGCATTAAGGCCTGGCGGTGTA |
| | | CTTTGCAACATGGCAGAGAGCATGTGGCTTCATACTCATC |
| | | TTATTCAAGATATGATCTCAATTTGCCGTGAAACATTTAG |
| | | TTCTGTTCATTATGCATGGGCCAGTGTTCCTACATATCCT |
| | | AGTGGCGTTATTGGGTTTCTTCTGTGCTCAACTGAGGGAC |
| | | CATTIGTTGATTTTAAGCACCCTGTTAATCCTATTGAGAA |
| | | GTTAGAGGGCGCTCTCCAGCATCAGCGGGAACTCAAGTTT |
| | | TACAATTCTGAGATGCATGAAGCTGCTTTTGCATTGCCAT |
| | | GCTTTTTAAGGAGGGAGGTGAGCGGTCTCCGCGATAGCCC |
| | | ACGTTCCGCAGGGAGTGGAGTCAAGATTTAA |
| SEQ | Solyc03g080100.4 | ATGGGTGAAGAAACGAAACAGGAGCCACCAAAGGAAGAAG |
| ID | | TAAAAACAGAGGAAGAGCAAACAGAGCCAAAACCACCTTC |
| NO: | | GCCTTTTGTTTTGTACGTTGACTTGCATTGTGTTGGATGT |
| 166 | | GCAAAGAAAATTGAGAGGTCCATTTCAAAGATTAGAGGGG |
| | | TTGAGGGAGTAGTAATAGAAATGGCGAAAAACACAGTGAC |
| | | AATAAAGGGAGTAATAGAGCCACAAGCAGTGTGCGACAGA |
| | | ATTACGAAGAAAACTAAAAGAGTTGCAAAAGTGTTATCAC |
| | | CCCTGCCGGCTGCTGAAGGTGAACCAATTCCTCAAGTGGT |
| | | TGCTTCACAGGTAAGTGGATTGACTACTGTGGAGCTTAAT |
| | | GTCAACATGCATTGTGAGGCTTGCGCCGAGCAACTTAAAA |
| | | AAAAGATTCTCAGAATGAAAGGTGTTAGAAGTGCGGAAAC |
| | | AGAAGCCAGCACTGGCAAAGTAACGGTGACGGGCACTATG |
| | | GACGCTAACAAACTCGTCGACTACGTCTATCGACGCACCA |
| | | AAAAACAAGCCAAAATTGTCCCTCAGCCCGAACCTGAGCC |
| | | CGAAAAGCCCGTTGAAGAGACCAAGCCCGACGAGGCGAAG |
| | | CCCGTTGAAGAACCAACGCCCGAAGATAAGAAAGATGAAG |
| | | GTACACCCCCGACTGAAAATAAAGAAGGGGGCGATGGTGA |
| | | AAAACCACCGGAGGGAGAAAAAAACGAAGGCGGTGGTGGA |
| | | GGAGAAGCGATGATCATTCCAATTGAAGATCATTATGATG |
| | | AACATACGATTAATAAGTTGATGTACTATCATCCTTATCA |
| | | GCCACTTTACGTCATTGAAAGAATTCCACCTCCGCAGTTA |
| | | TTCTCAGATGAAAATCCCAATGCATGTTGCATTACATAA |
| SEQ | Solyc03g111010.4 | GTGAAAATCCTCAACGACCGCATACTTCGGTATCCGGTCA |
| ID | | CCAACCAGTTTATATTTAAAGCCTTCATCACCACCAACAA |
| NO: | | CTCCACTACTCTGTTCTACAAAACTCTGCATCAACTCAAA |
| 167 | | TACACACAAAAAAAATCAATGGCTAAAGTTAAGATTGGAA |
| | | TTAATGGATTCGGAAGAATTGGCCGATTGGTTGCTCGGGT |
| | | TGCTCTCCAAAGAAATGATGTTGAACTCGTCGCAGTGAAC |
| | | GATCCCTTCATTTCAGTTGATTACATGACATATATGTTTA |
| | | AGTATGATAGTGTACACGGCCAGTGGAAGCATCATGAGCT |
| | | CAAGGTTAAAGATGAGAAGACTCTTCTCTTTGGTGAGAAG |
| | | GCTGTTACTGTTTTTGGGTTTAGGAACCCAGAGGAAATTC |
| | | CATGGGCACAGACTGGAGCTGATTACATTGTGGAGTCGAC |
| | | CGGTGTTTTCACTGATAAGGACAAAGCTGCAGCTCATTTG |
| | | AAGGGTGGCGCCAAGAAAGTTATCATTTCTGCCCCTAGTA |
| | | AGGATGCTCCTATGTTTGTTGTTGGTGTCAATGAGAAGGA |
| | | ATACAAGCCAGAGCTCAATATCGTTTCAAATGCTAGCTGT |
| | | ACCACAAATTGCCTTGCTCCCTTGGCCAAGGTTATAAATG |
| | | ACAGATTTGGAATTGTTGAGGGTCTCATGACCACAGTCCA |
| | | CTCCATCACAGCCACTCAGAAGACCGTTGATGGACCATCA |
| | | GCCAAGGATTGGAGAGGTGGAAGAGCTGCTTCGTTCAACA |
| | | TTATTCCTAGCAGTACTGGAGCAGCCAAGGCTGTGGGGAA |
| | | AGTGCTACCAGCATTGAATGGAAAGTTAACTGGAATGGCT |
| | | TTCCGAGTCCCAACTGTTGATGTGTCCGTGGTTGACCTCA |
| | | CAGTCAGGTTGGAAAAAGAAGCTACCTATGATGAAATCAA |
| | | AGCTGCCATCAAGGAGGAATCTGAAGGAAAATTGAAGGGA |
| | | ATTCTAGGTTACACTGAAGATGATGTGGTTTCCACAGACT |
| | | TTGTGGGAGACAATAGATCAAGCATATTTGATGCCAAGGC |
| | | TGGAATTGCTTTGAGCAAGAATTTTGTCAAGCTTGTAGCA |
| | | TGGTACGACAACGAATGGGGTTACAGTACACGAGTGGTGG |
| | | ACTTGATTATTCACATGTCATCAGTTCAGTAA |
| SEQ | Solyc03g112080.1 | ATGGGGTATGCAAATGGTCTTCTGTGTCCACTAGGGAAGC |
| ID | | AGCCACTTCTCTCTTTTGGTGTCATCTCAGATGTCCAGTA |
| NO: | | TGCTGATATTGATGACGGCACCTCATTCCTTGGTGTTCCT |
| 168 | | AGGTATTACCGGCACAGTGTTAGCGTGTTGCAAAGGGCAG |
| | | TTAAGAAATGGAATCAAGAAAAGCCTAAGTTTGTTCTTAA |
| | | TTTTGGGGACATTGTCGACGGGTATTGTCCCAAAGACCAG |
| | | TCAATGATTGCTGTCAAGAAAATTGTTGATGAATTTGACA |
| | | AATTCAATGGTACTGTCTATCACATGATTGGAAATCACTG |
| | | CCTTTACAATCTTCCTCGAAAAGACTTGCTTCCATTGCTG |
| | | AGAATCCCTGGCCATGATGATCATGCCTATTTTGATTTTT |
| | | CTCCAATTCCGGAGTACAGATTTGTAATCCTAGATGCTTA |
| | | TGATATCAGTGCCATTGGTTGGCCGGAAGATCACCCAAAT |
| | | ACTTTGAAGGCCTTGAAAGTTCTTCAGGAGAAAAACCCAA |
| | | ATTCAGACAAAAACAGTCCAAGTGGACTTGTGGGACCGGC |
| | | AAGAAGGTTTCTCATGTTCAATGGAGGTGTTGGGAAAGAA |
| | | CAATTGGAATGGCTGGATCATGTCCTTCAGGAAGCTACCA |
| | | AGTTGAATCAAAATGTAATAGTGTGTTGCCATTTGCCTTT |
| | | GGATCCTGGAGCTTCATCTCTTGCAGCATTGTTGTGGAAC |
| | | TATGATGAAGTGATGGATGTGATTCATCGCTATAGCTGTG |
| | | TGAAGGTCTGTATGGGTGGACACGACCATAAAGGTGGACA |
| | | ATCAGTCGACTCCCATGGTGTACATCATCGCGTCCTTGAA |
| | | GCAGCTCTTGAGTGCCCTCCTGGTACTGATTCTTTCGGAC |
| | | ATATTGATGCTTTTGATGATAGATTATTACTTTTTGGTAC |
| | | TGATAGAATGAAGAGCACTGAGATGGTTTTTCGTCACTAG |
| SEQ | Solyc03g112620.4 | ATGAATACTTCTTCTTCAATCATTTTCAAATTTGTTGCTT |
| ID | | TCCTTGTTATTTTTTCTTCATTTAGTACTCATGTTTTGTC |
| NO: | | TACTCTCCAAGCAAATCAAACATTCAGGCCAAAGGAAGAA |
| 169 | | TTGTTGAAACTCAAGAAAATAAAGGGTTATCTCAAGAAAA |
| | | TCAACAAGCGTCCTGTCAGAACGATTAAGAGTCCAGATGG |
| | | AGATTTAATAGATTGTGTTTTATCTCATCTTCAACCAGCT |
| | | TTTGATCATCCTCAACTTAAAGGAATGAAGCCATTGGAAC |
| | | CTCCAAAGAGGCCTAAAGCTAATGAATCAATGAATATAAT |
| | | GGAAGAAAGTTTACAACTTTGGACAGTATCTGGAGAATCA |
| | | TGCCCAGAAGGAACTGTTCCTATTAGGAGAACAACAGAGA |
| | | AAGATGTATTGAGAAGTAATTCATTAAGAAGATTTGGTAG |
| | | AAAAATTAGAAGAGGTGTTAGACATGACACATCAAGCAAT |
| | | GACCATGAGCATGCAGTAGCATTTGTGAATGGAGAACAAT |
| | | ATTATGGAGCAAAAGCAAGTATTAATGTATGGACACCAAG |
| | | AGTTACAGATCAATATGAATTCAGTCTGTCACAACTATGG |
| | | GTCATTTCTGGTTCATTTGGTAATGATCTTAATACCATTG |
| | | AAGCTGGTTGGCAGGTCAGCCCGGAATTATACGGAGACAA |
| | | CTATCCTAGGTTCTTTACTTATTGGACGACGGATGCATAC |
| | | CAAGCTACAGGATGTTACAACTTATTATGCTCAGGTTTTG |
| | | TTCAAATAAATAATAGAATTGCTATGGGAGCTGCAATATC |
| | | TCCAAGATCATCAGTTAATGGTAGACAATTTGATATTGGC |
| | | ATAATGATATGGAAGGATCCAAAGCATGGACACTGGTGGC |
| | | TAGAATTCGGGTCGGGTCTATTAGTTGGATACTGGCCAGA |
| | | TTTCTTGTTTAGCCATTTAAGAGGACATGCAAGTATGGTA |
| | | CAATTTGGAGGTGAAGTAGTGAATTCAAGATCAATGGGGT |
| | | ATCACACTTCAACACAAATGGGAAGTGGACATTTTGCTGA |
| | | TGAAGGATTTGGAAAAGCTTCTTATTTTAGAAATTTGCAA |
| | | GTTGTTGACTGGGATAATAACTTGATTCCTTTGTCAAATC |
| | | TTCATCTATTAGCTGACCATCCAAATTGCTATGATATTAG |
| | | AGCTGGAAAAAATAATGTTTGGGGTAATTATTTTTACTAT |
| | | GGTGGTCCTGGAAGAAGTTCAAGGTGTCCATAA |
| SEQ | Solyc04g005040.1 | ATGAGGAGGATTCATCTATACATCGCCATTGCTTATGTTG |
| ID | | TAATATTTCAAACTAGTTGTTCAGCTCATTTCTTTCCAAA |
| NO: | | TATTTCATCAATCCCTTCTTCATTACTCAAACCTAATGCC |
| 170 | | ACTGCTTGGACTTCTTTTCAAAAGTTGTTAGGATGTCAAC |
| | | CTGGTCAGAAAGTCGATGGCATTGCTAAAATCAAAAAATA |
| | | TTTTCAACATTTTGGTTACATTAATAATTTGACTAGTTTT |
| | | AATTTCACTGATGAATTTGATGATACTCTTGAATCTGCTC |
| | | TCAAGACGTATCAGAGAAATTTCAACCTCAAAGCCACCGG |
| | | TGTGCTCGATGCGCCCACCATTCAGCATCTCATAAAACCC |
| | | AGATGTGGAAATGCCGATCTAGTTAACGGTACTAGTACTA |
| | | TGAACGCTGGAAAGCCGCACACGGTGGCTCACTACTCCTT |
| | | CTTTCCTGGAAGACCAAAGTGGCCCGAGGGTAAGACTGAT |
| | | TTGACTTATGCCTTTCTACCGGCAAACAATTTGACGGATG |
| | | ATATTAAGAGTGTCTTCTCACGTGCGTTTGATCGGTGGTC |
| | | GGAGGTAACCCCGTTGAGCTTCACGGAAATACCGTCATTT |
| | | CAATCGGCTGATATCAAGATCGGATTTTTAACCGGAGATC |
| | | ACAATGATGGAGAGCCGTTTGATGGTCCGATGGGGACATT |
| | | AGCGCACGCGTTTTCACCGCCGGCGGGGCATTTTCACTTG |
| | | GACGGCGAGGAAAATTGGGTCGTCGACGGTGTGCCGGTAA |
| | | ATGAAGGGAACTTTTTTTCTATATTGTCGGCTGTGGATCT |
| | | CGAATCGGTTGCGGTTCATGAAATCGGGCATTTATTGGGT |
| | | TTGGGTCATTCATCGGTAGAAGATTCGATTATGTATCCGA |
| | | GTTTAGAATCGGGTATTCGAAGAGTGGAGCTGGTGGAGGA |
| | | TGATATTAAGGGGGTTCAGGAATTATACGGGTCAAACCCG |
| | | AATTATACTGGGACCAATACAACATTGACTCCGAGCGGCC |
| | | TGGATAATGATACGAATGGAGCCCCGATTCGTAGCTCAGT |
| | | ATGGATTCATGGGTTTTTATTGGTGGTTGGATTTTTCATT |
| | | TATTCAATTTAA |
| SEQ | Solyc04g014510.3 | ATGGCTCATCTTTCAGATCTTGTCAATCTTAATCTCTCTG |
| ID | | ATTCCTCTGAGAAAATCATTGCTGAATACATATGGATTGG |
| NO: | | TGGATCAGGAATGGATGTAAGGAGCAAAGCCAGGACTCTA |
| 171 | | TCTGGTCCTGTTGATGATCCTTCAAAGCTTCCCAAATGGA |
| | | ATTATGATGGTTCTAGCACAGGTCAAGCTCCTGGAGAAGA |
| | | CAGTGAAGTGATCCTATATCCTCAAGCAATTTTCAAGGAT |
| | | CCATTCAGGAGGGGCAACAATATCTTGGTCATCTGTGATT |
| | | GTTACACCCCAGCTGGTGAACCAATTCCAACAAACAAGAG |
| | | GCACAATGCTGCTAAAATATTTAGCAACCCTAATGTTGTT |
| | | GTTGAGGAACCATGGTATGGTCTTGAGCAAGAATACACCT |
| | | TGCTACAAAAGGAAATTAATTGGCCTCTTGGATGGCCTAT |
| | | TGGTGGCTTTCCTGGACCACAGGGACCATACTACTGTGGA |
| | | ATTGGATGCGGAAAGGCTTTTGGACGCGATATTGTTGATG |
| | | CTCATTACAAGGCATGTATCTATGCCGGGATTAACATTAG |
| | | TGGTATCAACGGAGAAGTGATGCCTGGACAGTGGGAATTT |
| | | CAAGTTGGACCTTCAGTTGGCATTGCATCAGGTGACGAGT |
| | | TGTGGGCAGCTCGTTACATTCTCGAGAGGATTACAGAGAT |
| | | TGCTGGAGTTGTTGTGTCATTCGACCCTAAACCTATTCCG |
| | | GGTGACTGGAATGGTGCAGGAGCGCATACAAATTACAGTA |
| | | CTAAGTCTATGAGAAATGAGGGAGGGTATGAAGTTATCAA |
| | | AAAGGCTATTGAGAAGCTTGGACTTAGGCACAAGGAGCAC |
| | | ATTGCAGCATATGGTGAAGGCAATGAACGTCGTCTCACTG |
| | | GAAGACACGAAACAGCTGACATCAACACATTCAAATGGGG |
| | | TGTTGCAAATCGTGGTGCATCTATTCGTGTGGGAAGAGAC |
| | | ACGGAGAAGGAAGGAAAGGGATACTTTGAGGACAGGAGGC |
| | | CTGCATCGAACATGGATCCATATGTTGTGACCTCTATGAT |
| | | CGCGGAGACTACCATCCTGTGGAACCCTTGA |
| SEQ | Solyc04g014570.3 | ATGTGCACAAAATTCGTGAAGGAAATGGAGAAGATGGCGT |
| ID | | TGTACGTCGTCGTTTTGCTAATCATCTGCTATGCATCGCC |
| NO: | | GGCGAGATCAGACGGTTCCGATCATAAGTATAAATCCGGA |
| 172 | | GATCAGGTTCCACTTTATGCTAACAAAGTCGGTCCGTTCC |
| | | ATAATCCGAGTGAAACATACCGCTTCTTTGACCTTCCTTT |
| | | TTGTACTCCAGATCATGTAAAAGAGAAGAAGGAAGCCCTT |
| | | GGTGAGGTCTTGAATGGTGACCGATTGGTCAGTGCCCCTT |
| | | ACAAGCTTGACTTTTTGTATGATAAGGACTCAGAAATAGT |
| | | TTGCAAGAAGAAGTIGTCCAAGGAAGAAGTTTCTCAATTC |
| | | CGCAGCGCTGTTGCTAAAGACTACTACTTCCAAATGTATT |
| | | ACGATGACCTGCCCATTTGGGGTTTCTTAGGGAAAGTTGA |
| | | TAAGGAAGGAAAGTCTGACCCCAGCGAGTACAAATACTAC |
| | | TTGTTTAAACATCTTCATTTTGAGATCTTTTACAATGATG |
| | | ATCGTGTGATTGAGATCAATGCACGAACTGATCCTAATGC |
| | | CCTGGTAGATATTACAGAGGATAAGGAGGTTGATGTTGAC |
| | | TTCATGTATACTGTAAAATGGAAAGAGACAAAAACACCTT |
| | | TTGATAAGAGGATGGAGAAGTACTCACAGTCTTCCTCGTT |
| | | GCCGCATCACTTGGAAATCCATTGGTTCTCAATTATCAAT |
| | | TCTTGTGTGACAGTTCTTCTCTTAACTGGTTTTCTTGCCA |
| | | CAATTCTTATGCGGGTCCTTAAGAATGACTTTGTCAAGTA |
| | | TGCACATGATGAGGAGGCGGCTGATGACCAAGAAGAAACT |
| | | GGGTGGAAATACATCCATGGTGATGTGTTTAGGTACCCAA |
| | | GTCACAAATCCTTGATGGCTGCAGCACTTGGGAGTGGCAC |
| | | ACAGTTATTCACATTGACCATCTTTATTTTCCTTCTCTCA |
| | | CTTGTTGGTGTTTTCTATCCATACAACCGTGGAGCTTTAT |
| | | TCACGGCACTGGTCGTCATATATGCTCTCACATCTGGTAT |
| | | TGCAGGCTATACTGCTGCCTCTTTCTATTGCCAGCTAGAA |
| | | GGGACAAACTGGGTCAGGAATTTGATATTGACCGGAGCCT |
| | | TGTTTTGTGGACCCCTGTTTCTCACGTTCTGTTTCCTTAA |
| | | CAGCGTAGCAATTGCATACAGTGCTACTGCTGCACTGCCT |
| | | TTTGGAACCATTGTGGTCATCTTTCTCATATGGGCTCTTG |
| | | TGACATCACCTTTACTTGTATTGGGAGGGATAGCTGGAAA |
| | | GAACAGCAGGGCTGAGTTCCAAGCTCCTTGTCGAACCACC |
| | | AAATACCCAAGAGAGATCCCACCATTACCTTGGTACCGCG |
| | | GAACTCTACCTCAGATGGCAATGGCTGGGTTTTTGCCTTT |
| | | CAGTGCCATATATATCGAACTTTACTATATATTTGCAAGT |
| | | GTTTGGGGTCACAGAATTTACACCATATACAGCATCTTAT |
| | | TTATAGTCTTCATCATTCTTATAATTGTGACTGCTTTTAT |
| | | TACCGTGGCCTTGACATACTTTCAACTTGCTGCTGAAGAT |
| | | CATGAATGGTGGTGGAGGGCTTTCCTTTGTGGTGGATCGA |
| | | CTGGACTTTTCATCTATGGCTACTGCTTGTATTACTATTA |
| | | TGCCCGTTCCGATATGTCTGGCTTCATGCAAACCTCATTC |
| | | TTCTTTGGATACATGGCTTGCATCTGCTATGGTTTCTTCC |
| | | TCATGCTGGGGACTGTGGGTTTCCGTGCAGCCCTATTTTT |
| | | TGTCCGTCATATATACCGGTCAATCAAGTGCGAGTAG |
| SEQ | Solyc04g050990.3 | ATGGCGGTGGAGAGATTCGCCTGCGGCGACGAACTGCCGG |
| ID | | AAGGTTGTATAGCTACGGCACTGTCGTTGACGAGCCCCAA |
| NO: | | GGACGCCTGCCGGCTGGCGCTGGTGGCTTCAACTTTCCGG |
| 173 | | TCAGCAGCTGAATCCGACGCCGTTTGGGAACGGTTTCTGC |
| | | CGCCGGATTATTATGATATCATTTCCCGGTCCATTGACGG |
| | | TCCAGATTCACTTAATTTCGGTTCAAAGAAAGACCTTTAC |
| | | TTATACCTATGTGATAACCCTATTCTCATCGACGGTGGTA |
| | | CCAGGAGCTTCTCATTGGAGAAAGGGAGTGGAAAGAAATG |
| | | TTACATGTTAGCTGCAAGGTCGTTGCAAATTGTTTGGGTC |
| | | GATACGCCTAGGCATTGGAGATGGATTTCCCTTCCTGAGT |
| | | CGAGGTTTTCAGAAGTTGCCGAGCTTCTTGATGTCTGCTG |
| | | GTTTGACATATCTGGCAAAATAAACACAAATATGCTCTCC |
| | | CCTGACACTACATATGGCGCTTACCTTGTGTTCACGACAA |
| | | AAACCAGGACCTGTGGATTTGAAAACCAACCTGCAGAGAG |
| | | TTCAGTAGGGATTGCTGGGCATGAAAGATATACACATACA |
| | | TTTTATTTGAACCCTCAGGGAGGAAGGAGACAGTACAGAT |
| | | ACCAAGTAGTCCCGCGACGGTTAGGAATATTCACCCACCA |
| | | CATGGCTCGCATATTGAGAGAGGAAACGGAAGATGCACCA |
| | | AAAGGGTGTCGTCCTAAGCAGAGAAGTGATGGGTGGATGG |
| | | AAATAGAGTTGGGAGAATTCTTTGTCAAGAGGGGACAAGA |
| | | AGCTGAAGTAGAGATTAGTTTAACAGAGGTAAAGGGAGGT |
| | | GACAGGAAAGCTGGCTTAATTATTGAAGGGATTGATATCA |
| | | GGCCTAAGGAGTAA |
| SEQ | Solyc04g071890.3 | ATCATTGTACAAATTAACTATCTAGCTATAGCCATGGTGT |
| ID | | CAACTACTCTATCAAGTTTTCTACTTGTTCTTGTTCTTTC |
| NO: | | TTTCTTTGTCTCAGAAGCTCAAAGGCCTGCTTTAACTAAA |
| 174 | | GGTCTTTCATGGTCATTTTATCAATCTAGTTGTCCTCAAC |
| | | TTGAATCCATTATTAGAAACAGGCTTCAAAAACAAATCAA |
| | | GGATGATGTTGGCCAAGCTGCTGGCTTACTTCGTCTTCAT |
| | | TTCCACGATTGCTTTGTTCAGGGGTGTGATGGTTCAGTGT |
| | | TGCTAGATGGATCAGCAGGAGGGCCAAGTGAGCAAACTGC |
| | | AATTCCAAATTTGACCCTAAGAAAAAGGTCATTCAAGATA |
| | | ATTGATGATCTTAGAAAAAGGATCCAAGATGAATGTGGTC |
| | | AAGTTGTGTCTTGCTCTGATATTGTTGCCATTGCTGCTAG |
| | | GGACTCTGTTGTCTTGACGGGTGGGCCCAACTACGATGTA |
| | | CCCTTAGGAAGAAAGGACGGAGTCAACTTTGCAACGGAGC |
| | | AAGCGACAATTGACAACCTAGTCGCACCCTCTGCCAACAC |
| | | CACAACCGTCCTCTCTCTCCTCGCAACCAAAGGCCTCGAT |
| | | GCGACCGATGCGGTTGCGTTATCTGGGGCCCACACTATTG |
| | | GTATCAGCCACTGTCCTTCCTTCACCGACCGTCTCTACCC |
| | | AAACCAAGACTCCACTATGGACAAGACATTTGCTAACAAT |
| | | CTTAAAGGTAGTTGTCCCACGGCTGACTCGAACAACACGG |
| | | TCAACATGGACATTCGTAGCCCTAACGTTTTCGACAACAA |
| | | GTACTACGTTGATCTCATGAATAGACAAGGGCTTTTTACG |
| | | TCCGATCAAGATTTGTATACGGATCGAAGAACACGAGGGA |
| | | TTGTAACGAGCTTTGCGGTGAATCAATCGTTGTTTTATGA |
| | | AAAATTTGTTATTGGTATGATAAAAATGGGACAAATGAAT |
| | | GTGTTGACTGGTGGACAAGGTGAAATTAGGAATAGGTGTG |
| | | ATAGGAGGAATAAGGCTAAAAAGGTTGATATTGCTACTGT |
| | | TGTTGAAGAATTGGAGGAAACTTTTTCTGCTTTGTTTTAA |
| SEQ | Solyc04g077190.4 | ATGAAGAGGTTATGTGCTTTCAGCTTTGCTCGTCGACTCT |
| ID | | TCAAACCTCGTACAACACATTCCCAGGAATCAAAGGAGAA |
| NO: | | AATGGAGCAATCACCAATTGTCAATGTCGAGGACAACTTT |
| 175 | | GATTTGCAGGAATCAAAGGAGAGCATGGAGAAGTCACCAA |
| | | CGGTCACTGCCAACAACAACTTTGATACTCAGAGTGCAAC |
| | | AGAAAATGAGAAAGAGAATGGGAGTTATGCTGCTACTAAC |
| | | ATCATCCTAAACCATGAATTCTCGGATGGGCTGCACTCGT |
| | | GGCATCCCAATTGTTGCGATGCTTTTGTGGTTCCAGCAGG |
| | | TTCTGGATACCATAAAGGATTAGCGGCGAAGGAAGGCTGT |
| | | TGTTATGCTGTTGTCTCAAATCGTAAGGAATGTTGGCAAG |
| | | GCTTAGAGCAAGACATTACAAGCAGGGTATCAGCAGGTTC |
| | | CACTTATACAGTTTCTGCTTGTGTTGGGGCCACTGGTACT |
| | | TTTCAGGGTTCTGTTGAAGTCCTCGCCACGTTAAAACTAG |
| | | TGTATCAAAATTCAGAAACAAGTTATCTATTCGTTGCAAA |
| | | AAAAGCTGCTTCGGAGGAGTGTTGGGAGACATTAGAAGGT |
| | | TCATTTTCTCTGTCAACTATGCCCGATCAAGTTATATTCT |
| | | ATCTCGAGGGACCTCCAGCTGGAACTGACCTGCTCATAAA |
| | | GTCAGTAGTGATATCATGTCCTAGTTCAACTGCTTCTGAT |
| | | AGTTCTGGCACATCATCTGTATATATTGATGATGATAACA |
| | | TCATAATAAACCCTCAATTTGATGATGGCCTCAATAGTTG |
| | | GTCTGGAAGAGGCTGCAAGGTGGCCTTGCATGACTCTATG |
| | | GCAGATGGCAAGATCACTCCAATGTCTGGAAAATCTTTTG |
| | | CATCTGCAACAGAACGCACCCAGAGCTGGAATGGAATTCA |
| | | GCAAGATGTGACAGGAAGAGTGAAGCGAAAACTAGCTTAC |
| | | GAGGTATCTGCTGTTGTCCGTATCTATGGTAACAATGTCA |
| | | CTACTGCAGATCTTCGCAGTACATTATATGTTAAAGCAGC |
| | | TGACAACCGTGAACGGTATATAGGCATTGCCAGCGTCCAA |
| | | GCCACAGATAAAGACTGGGTGAAGTTGCAAGGGAAGTTTC |
| | | TTATAAATGACTCCCCGTCTCAGGTTGTTGTCTTTTTAGA |
| | | AGGTCCACCTGCAGGAACGGATATCCTCCTTAACAATTTA |
| | | GTCATAAAGCATGCAGCTAAAGCTCCTCCTTCTTCTCCAC |
| | | CAGTTATTGAGGATGCTGGTTTTGGTGTTAATATAATCAC |
| | | AAATACAAGTTTGAATGATGGCACAAATGGCTGGTTTCCA |
| | | CTTGGAAACTGTACAATGAGTGTTCAAACAGGCTCACCAC |
| | | ATATTATGCCTCCTATGGCTAGAGATACTCTTGGTGCTCA |
| | | TGAGCCTTTAAGTGGCCGTTATATTCTGGTGGCTAATCGT |
| | | ACTCAAAACTGGATGGGTCCTGCTCAGATGATCACGGAAA |
| | | AAGTAAAACTCTATTTGACATACCAAGTATCTGCATGGGT |
| | | TAAAATTGGACAAACATCAGGACCTCAGAATGTAAATGTT |
| | | GCCCTTGGAGTAGACAGCCAATGGGTAAATGGAGGCCAAG |
| | | CTGAGATCAGCGATGATAGATGGCATGAAATTGGAGGATC |
| | | TTTTAGAATTGAGAAGCAAGCTGCTAAGATTATGGTTTAT |
| | | ATTCAGGGTCCTGTTGCTGGTGTAGACTTAATGGTTGCTG |
| | | GGCTTCAAATTTTTCCGGTTGACAGACGTGAACGGTTTAG |
| | | ACACTTGAAGAAACAAACTGCAAAGTTACGCAAGCGAGAC |
| | | GTCATGTTGAAGTTCTCTGGATCAGATTCAGGCAATTTGT |
| | | TTGGCACATTTGTTAGAGTTAAACAACTGCAAAACAGCTT |
| | | TCCCTTCGGATCAGCCATAAGCAGAACAAATATGGACAAT |
| | | GAAGACTTCAATGCCTTCTTTGTCAAGAATTTCAATTGGG |
| | | CTGTGTTTGGAAATGAGCTCAAATGGTACTGGACAGAAGC |
| | | ACAGCAAGGGAATCTCAACTACAAAGATGCTGATGAGCTC |
| | | CTGGACTTTTGCACAAAAAACAACATCCAAGTTCGAGGTC |
| | | ACTGTATCTTTTGGGAGGTGGTGGGCACGGTTCAGGCATG |
| | | GGTACAATCCTTGAACAAAAATGACTTGATGACGGCTGTT |
| | | CAAAACCGTTTAACAGGGCTGCTGACACGGTATAAGGGGA |
| | | AGTTCCCGCATTATGATGTGAATAATGAGATGATGCATGG |
| | | TTCTTTCTATCAAGATAGATTGGGTAAAGACATCAGGGTA |
| | | AACATGTTTAAAACTGCACATCAATTGGACCCTTCCCCCA |
| | | TCCTATTTGTAAATGATTACCATGTTGAGGATGGCTGTGA |
| | | CACCCGATCTTACCCTGAGAAATACATTGAGCACATTCTT |
| | | GATCTCCAAGAGCACGGTGCACCTGTTGGAGGTATAGGTA |
| | | TTCAGGGACATATTGACAGTCCAGTTGGTCCCATTGTATG |
| | | TTCTGCTCTGGACAAACTTGGTACTCTTGGACTTCCAATC |
| | | TGGTTCACTGAGGTTGACGTGTCTTCAGATAATGAATACG |
| | | TTAGAGCTGATGATCTAGAAGTTATGCTTCGAGAAGCTTA |
| | | TGCTCACCCTGCTGTAGAAGGTATAATGTTGTGGGGATTC |
| | | TGGGAGCTGTTCATGAGTCGAACAAATGCACATTTAGTGG |
| | | ATGCAGAAGGAGACATCAATGAAGCTGGAAAAAGGTACCT |
| | | TGCTCTTAAGCATGAGTGGTCATCTCATCCGCATGGTCAT |
| | | ATTGATGAACAAGGGCAATTCAGCTTTAGTGGATTCCATG |
| | | GCTCCTATGAAGTGGAAGTCGTAACAGTTTCAAAGAAAAT |
| | | TACTAAGAAATTTGTGGTTGACAAGGGCGATAATGCACTG |
| | | AGTTCAAAGGAAAATGGAAACATCATCCTTAACCATGAAT |
| | | TCTTGGATGGTCTGCACTTGTGGCGCCCAAATTGCTGTCA |
| | | TGCTTTTGTGGTTCCAGCAGGTTCTGGATACCGTAATGGA |
| | | TTGACTATGGCTGTTGTGACAAAAAGTACGATATGGTGGC |
| | | AAGGCTTAGAGCAAGACATTACAAGCAGAGTATCAGCAGG |
| | | TTCCAGTTGTACTGCTTCTGATGAATCAAAGCAGGAAATG |
| | | GAGAAGGCACCGCCAACAATTACTTTGATTCTCAGTTCAA |
| | | AGGAAGATGAGAAAGACACTGGGAACATCATTTTAAACCA |
| | | TGACTTCTCATATGGGCTTTACTTGTGGAACCCAAATTGC |
| | | TGTGAGGCATTTGTGGTTCCATCAGGTTACCATAAAGGAT |
| | | TAGCAGCTGCTATTGTAACAAATCGTAGAGAATGTTGGCA |
| | | TGGCTTGGAGCAAGACATTACAAGCAAAGTATCAGAAGGT |
| | | TCTACTTATACAGTTTCTGCTTGTGTTGGAGCCTCTGGTA |
| | | CTTGTCCGGGTTCTGTTGAACTGACCTGCTCATAA |
| SEQ | Solyc04g078390.3 | ATGAAGCGGCAATTCGACGCCGGAGAAACCGATTGGGCCA |
| ID | | ATTTCACCGGCGGCGATCATGTGAAGAAGATGAAGAAGGA |
| NO: | | GATGTTAGAGTTGGAGGAAGAGAGGAAGTTACCGATGAAG |
| 176 | | AGGCAATTTGAAGCTAACTGTGCAGCTAAAGTCTCCGGCG |
| | | ATTTGAAAAAGATGAAGCAGGTTATAGAGTCGCCGGATGC |
| | | GGATGCGGATGCGGATACGGAGGCGGTGTTGTTGAATGAG |
| | | AATCTACTGTACGAAGTACTGAAACACGTTGACGGGCGCA |
| | | CGTTAGCTACGGCGGCGTGCGTTAGCAAACAGTGGAATCA |
| | | AACGGCCCTAGATGAGCGGCTATGGGAGCTGATCTGCACA |
| | | AGACACAGCTGTAACCAACAGCAACAGCTCCGAGCCGTCG |
| | | TCCTCGCTCTCGGCGGCTTCCGGCGTCTCTACTCACTCTA |
| | | CCTCTGGCCACTCTCAAAGCCGTCGCCGTCTCCATCGGCG |
| | | TCTTCTTCGAATCCACCGGCTTCCGCCTGGCCTTGCCTTC |
| | | CTCCGGCGCCTACTCGGCCGTTGAAATACGCTGCTGCAAA |
| | | CACCAGATGGGGAAAAGACGAAGTTAATCTCTCGCTATCG |
| | | CTGCTATCAATTCGGTACTACGAGAAGATGAATTTCAAAG |
| | | CTAATAAATAA |
| SEQ | Solyc04g078540.4 | CCATCTTCTTTTTCCATACCTCAACTAAAACAATCAAACA |
| ID | | TGGCGATTCATCTCTCCGCTCTAACAATCTCGATCCTCGT |
| NO: | | AATGGTCGTGTCCTCCGCCGCCGTAACATCGGCGGCGGCA |
| 177 | | GAGGACATGTCGATTATAAGCTACAACGAAAAACATCACA |
| | | CGATCGGCGCCGGTCGGACCGATGACGAGGTCATGTCTAT |
| | | GTACGAATCGTGGTTAGTTGAACACAAAAAAGTGTACAAC |
| | | GCTTTGGGAGAAAAAGATAAACGGTTCCAGATCTTTAAGG |
| | | ACAACCTTAAATACATCGATGAACATAACGCTATGCCGGA |
| | | GAAAAGTTATAAGCTCGGTTTGACTAAGTTTGCTGATCTG |
| | | ACCAACGAGGAGTATAGGTCGGTTTATTTGGGTACGAAGC |
| | | CGGATGCTAGTCGCAGGTTGTCGAGTAGACAAAGTGATCG |
| | | GTATGCTCCGAAGGTCGGAGATAGGTTGCCGGAATCTGTT |
| | | GATTGGGTGAAGAAAGGTGTGCTTGTTGGTGTTAAGGATC |
| | | AAGGACAATGTGGGAGTTGCTGGGCTTTCTCAGCAGTTGC |
| | | TGCCATTGAAGCAGTAAACAAAATAAAGACTGGAGACTCG |
| | | ATCTCTTTATCCGAGCAGGAGCTAGTAGATTGTGATACTT |
| | | CTTCTAACAATGGTTGTGACGGTGGTCTCATGGACTATGC |
| | | TTTTGAATTCGTTATCAAAAATGGAGGACTAGACACTGAG |
| | | GAAGATTACCCATATACTG |
| | | GCGAGGATGGAAGATGTGACCTAACAAGGAAAAATGCCAA |
| | | GGTTGTCACCATTGACGGGTATGAAGATGTTCCTGCTAAT |
| | | GATGAAAACGCAATGAAAAAGGCTATTGCAAGTCAGCCTG |
| | | TTAGCGTTGCTATTGAAGCTGGTGGCAAAGACTTCCAGCA |
| | | CTATAAATCGGGCATCTTTACTGGGAAGTGTGGCGCAGCA |
| | | GTGGACCATGGTGTGGTTGCAGTGGGATATGGTAGCGAAA |
| | | ATGGCATGGATTATTGGATTGTGAGGAACTCATGGGGTGC |
| | | TTCCTGGGGAGAACACGGCTACCTCAGGATGCAGCGTAAC |
| | | ATTGCTAACCCAAAGGGTTTGTGTGGTATTGCTACAGTTG |
| | | TTTCTTACCCTGTCAAGACAGGCCAAAATCCCCCGAAACC |
| | | AGCTCCATCTCCTCCATCGCCAGTCAAGCCACCTACTACT |
| | | TGTGATGATATGTACAGTTGCCCATCAGGAACAACGTGTT |
| | | GCTGTGTCTACGAGTACTATCACATGTGCTTTGCTTGGGG |
| | | TTGCTGCCCTATGGAAGGAGCTACTTGCTGTAAAGACCAT |
| | | AACAGTTGCTGCCCACATGATTATCCTGTCTGCAATGTTA |
| | | AAGCCGGCACCTGCTCAATCAGCGAGAATAACCCTCTTTC |
| | | AGTCAAAGCAATGTCACACATTCTTGCCAAACCTATTGGA |
| | | TCCTTCAGCAATCAGGGAATGAAAAACACTATCTCTTGA |
| SEQ | Solyc04g078760.4 | ATATTGAAAATTGAAAACAACACCACCCAATTTTTCTCTC |
| ID | | TCTTCTTCTTTTCCCCCCTTTCACTTTCTCTCTCTAAAAA |
| NO: | | CCTAGCTATGGCGCTTCCTCATCACCATCTCCAACTACAC |
| 178 | | ATTCAACAACAACCACATCAACAACAGCAACAGTCCAAAT |
| | | CTTACAGAGATTTGTACAACAATATGGATGGTCAGATAAC |
| | | AAACCCAGTTGTTTATTTCAATGGCTCTAATCTTCCTGAA |
| | | CAGTCTCAACACCCTCCTTATATACCTCCCTTTCAAGTTG |
| | | TGGGGTTGGCTCCTGGTACAGCTGATGATGGAGGGTTGGA |
| | | TTTGCAGTGGAATTATGGGTTGGAGCCTAAGAAGAAGAGG |
| | | CCTAAAGAGCAAGATTTTATGGAGAATAACAACTCTCAAA |
| | | TATCTTCTGTTGATCTGTTGCAGCGGCGATCGGTGTCCAC |
| | | TGGCTTGGGGTTGTCTTTGGATAATGGACGTTTGGCTTCG |
| | | TCGTGTGACTCAGCGTTTCTAGGGCTCGTGGGTGATGATA |
| | | TTGAGCGAGAATTGCAGAGGCAAGATGCTGAGATTGATAG |
| | | ATATATCAAAGTTCAGGGTGATCGCTTAAGGCAAGCTGTA |
| | | TTGGAGAAGGTTCAAGCTAATCAGATTCAGGCTATCACCT |
| | | ATGTTGAAGAGAAGGTCCTTCAGAAACTTCGAGAAAGAGA |
| | | CACAGAGGTTGATGATATCAACAAGAAAAACATGGAACTT |
| | | GAGTTGCGGATGGAACAATTGGCTTTGGAAGCCAATGCTT |
| | | GGCAACAGCGTGCCAAATACAATGAGAACTTGATTAACAC |
| | | ACTTAAGGTAAATCTACAACATGTTTATGCACAGAGCAGA |
| | | GATAGTAAAGAAGGTTGTGGTGACAGTGAGGTAGATGACA |
| | | CAGCATCATGCTGTAATGGACGGGCTACAGATCTCCACCT |
| | | GCTTTGCCGGGATAGCAAGGAAATGAAGGAGTTGATGACG |
| | | TGTAGGGTCTGTAGAACCAATGAAGTTTGCATGCTATTGT |
| | | TACCATGTAAGCATCTCTGCCTGTGTAAAGAGTGTGAGAG |
| | | TAAGCTTAGTCTTTGTCCCTTGTGTCAGTCCACTAAGTAT |
| | | ATAGGTATGGAAGTTTACATGTAG |
| SEQ | Solyc04g080190.3 | ATGAAGGCTTCATTCAAAGCACGATATGAGCCCGACAAAG |
| ID | | CCGCCGCCAACGCTACTGTTGCATTCAACGCCGGCGACCT |
| NO: | | TAAGCTCCGAGCCTCCATGACCGATGCTACTGTTGTCAAA |
| 179 | | GGTCCCAGCTTAAACGGATTAGCTCTGGCCGTTGAGAAAC |
| | | CTGGATTGTTCATCGTCGACTACAACGTCCCGAAGAAGGA |
| | | CATTAGGTTTCAATTTATGAACTCAATCAAGGTTCTGGAG |
| | | AAGCCATTGAATTTGAATTACATTCATTTTCATGGAGACA |
| | | AGCGAACAATATTGGATGGAACTTTGGTGGTTGATTCTGC |
| | | CAACAAGGTGTCGGCTAATCACGTCATGGGCTCAGGAAGT |
| | | TGTAAATTAAAGTACACATATGTCCACGGAGGAATCACTA |
| | | CTTTCGAGCCCATCTATGATACAGCAAAGAATGCGTGGGA |
| | | CTTCATGGTATCACGTAAAGTTTATGGGGATGACGTGTTT |
| | | AAGGCTACTTATCAGACAACAAGCAAAAATTTGGGGCTCG |
| | | AATGGTCCAGGAGTTCTAAGTTAAATGGATCATTTAAGAT |
| | | TTGTGCATCTCTCTGTTTGCTTGAGGAACGCAAAATCCCA |
| | | AAGTTAAGTGCTGAGACGTCGTGGGACTTTGAAATGTGA |
| SEQ | Solyc04g080960.4 | ATGTTAAACGCCGAGCATCACTTTTCACTTTTTAAGGCTA |
| ID | | AGTTTGGAAAGATCTATGCTTCTCAGGAGGAACATGACCA |
| NO: | | TAGATTGAAGGTATTCAAGGCTAATCTTCACCGTGCGAAG |
| 180 | | CGTCACCAGCTCCTTGACCCTTCCGCTGAGCACGGTATTA |
| | | CGCAGTTCTCCGATTTGACTCCGTCGGAGTTTCGCCGGAC |
| | | TTACCTTGGACTTAACAAGCCCCGACCTAATCTTAATGCC |
| | | GAGAAAGCTCCAATCCTCCCGACTAAAGATCTTCCATCTG |
| | | ATTTTGACTGGCGTGAAAAAGGAGCCGTCACCGATGTTAA |
| | | AAATCAGGGCTCATGTGGATCATGCTGGTCATTCAGTACG |
| | | ACTGGAGCAGTGGAGGGAGCTCACTTTTTGGCGACCGGAG |
| | | AGCTTGTGAGCCTTAGTGAACAGCAGCTTGTGGATTGTGA |
| | | CCATGAGTGTGACCCGGTGGAAAAAAATGATTGTGATGCA |
| | | GGTTGCAACGGTGGTCTTATGACTACTGCCTTCGAGTATA |
| | | CCCTTAAGGCTGGAGGTCTCCAACTGGAGAAAGACTATCC |
| | | TTACACTGGCAGGAATGGCAAATGCCACTTTGACAAGAGC |
| | | AGAATTGCTGCCTCTGTATCCAACTTCAGTGTTGTTGGTC |
| | | TTGATGAAGACCAAATTGCTGCTAACCTGCTGAAACATGG |
| | | TCCTCTTGCAGTGGGGATCAATGCTGCTTGGATGCAGACA |
| | | TATGTTAGAGGAGTTTCATGCCCATTAATTTGCTTCAAGC |
| | | GTCAGGATCATGGTGTCCTCCTAGTCGGTTATGGATCTGA |
| | | AGGTTTTGCACCTATACGTCTCAAGAATAAGCCTTACTGG |
| | | ATCATCAAGAATTCTTGGGGAAAGACTTGGGGAGAACACG |
| | | GATATTACAAGATCTGCAGGGGTCACAATATCTGCGGAGT |
| | | TGATGCCATGGTCTCTACTGTGACTGCCACACATACTACT |
| | | AATCCTAATCTTTAA |
| SEQ | Solyc04g082030.1 | ATGGCCGGCCAAACAGTCATTGTTTCCGGTTTGAATCCGG |
| ID | | CGGCCATTCTTCAGTCCACAATCGGTGGAGCTCCGGTGGC |
| NO: | | AGCGGCGGCGGAGAACGGCCATACGAGAAAAGTTGTTCCT |
| 181 | | CTGTCAAAAGATGCCCTTCAGGATTTCATGGTTTCAATCA |
| | | TAACACAGAAATTACAGGACGATAAACAACCCTTTTATGT |
| | | TCTAGATTTGGGTGAAGTTGTTTCCCTTATGGAACAATGG |
| | | AACTCTGCTTTACCAAATATCCGTCCGTTTTATGCTGTTA |
| | | AATGTAACCCTGAACCTTCATTTCTCTCCATGTTGTCTGC |
| | | TATGGGTTCCAATTTTGATTGTGCTAGCCGTGCTGAAATT |
| | | GAGTATGTTTTGTCTCATGGTATCTCGCCGGACCGGATTG |
| | | TTTTCGCTAACCCTTGTAAGCCGGAATCCGATATTATCTT |
| | | CGCCGAGAAAATTGGGGTTAATCTCACTACTTACGATTCC |
| | | GAGGATGAAGTTTACAAGATCCGTAAACATCACCCAAAAT |
| | | GCGAGCTTTTGCTTCGAATCAAACCCATGACCGACGGCAA |
| | | CGCGAGATGCCCAATGGGTCCAAAATACGGCGCATTACCG |
| | | GAGGAAATCGAGCCGCTCCTCCGTACAGCTCAAGCCGCTA |
| | | GACTCACCGTCTCCGGCGTGTCCTTCCATATCGGCAGCGG |
| | | AGACGCCGATTCCAACGCTTATCTAGGAGCCATAGCCGCA |
| | | GCGAAACAGGTTTTCGAAACAGCAGCTCAACTCGGAATGC |
| | | CAAAAATGACGGTACTCGACATCGGCGGCGGATTTACCTC |
| | | CGGCCACCAATTCACCACCGCTGCTCCCGCCGTTAAATCA |
| | | GCTCTGGAAACCCACTTCCACGACTTCCCTGAGCTAACCA |
| | | TCATCGCTGAACCAGGTCGTTTCTTCGCCGAAACAGCGTT |
| | | CACTTTAGCAACAACCATCATCGGCAAAAGAGTCAGAGGT |
| | | GAATTAAAAGAATACTGGATCAACGATGGGTTATACGGAT |
| | | CGATGAACTGTGTACTTTACGACCATGCGACGGTGACGGC |
| | | GACGCCATTAGCGTGCATGTCGAATCGTAATAACCTCAAC |
| | | TGCGGCGGGTCGAAAACGTTTCCGTCGACTGTATTCGGGC |
| | | CCACCTGTGATGCACTTGATACCGTGTTAAGGGATTACCA |
| | | GTTACCGGAGCTACAGGTTAATGACTGGCTGATTTTTCCT |
| | | AATATGGGTGCTTATACAAAAGCTGCTGGATCAAATTTCA |
| | | ATGGATTTAACACATCCGCCATTGTTACTCACCTTGCTTA |
| | | TGCTTATCCAAACTAA |
| SEQ | Solyc04g150161.1 | AACATGGCTAAAATAGCCTATCTTCTTACCACTCTTTTAG |
| ID | | TGGCTTTAGTGTCACTTAGCTTCCCTTCAGAATGCAAAGC |
| NO: | | AAATTACTACTACACATCTCCACCTCCACCAACTCCAGTT |
| 182 | | TATAAGTATAAGTCTCCGCCACCACCAGTTTATAAGTACA |
| | | AGTCACCACCACCACCTCCTCCAATCTATAAATCCCCACC |
| | | GCCACCCATTTATAAGTATAAATCTCCACCACCACCTCCA |
| | | GTTTACAAGTACAAATCTCCACCACCACCCGTCTACAAGT |
| | | ACAAGTCTCCTCCACCACCACCTCCAGTTTACAAGTACAA |
| | | ATCTCCACCACCACCTGTCTACAAGTACAAGTCTCCTCCT |
| | | CCTCCACCTCCAGTTTATAAGTACAAATCTCCACCACCAC |
| | | CCATCTACAAGTACAAGTCTCCCCCACCACCCGTTTACAA |
| | | GTACAAGTCTCCACCACCACCTGTCTATAAGTACAAGTCT |
| | | CCACCTCCACCTGTCTACAAGTACAAGTCTCCACCACCAC |
| | | CAGTATACAAGTACAAGTCTCCACCACCTCCAGTATACAA |
| | | GTACAAGTCTCCACCACCTCCATCTATAAGTACAAGTCTC |
| | | CACCTCCACCCGTTTACAAATACAAGTCTCCACCACCTCC |
| | | AGTCTATAAGTACAAGTCTCCACCTCCACCCGTTTACAAA |
| | | TACAAGTCTCCACCACCTCCATTCTACAAGTACAAGTCTC |
| | | CACCTCCACCCGTTTACAAATACAAGTCTCCACCACCTCC |
| | | AGTCTATAAGTACAAGTCTCCACCACCTCCAGTCTATAAG |
| | | TACAAGTCTCCACCACCTCCAGTCTACAAGTACAAATCTC |
| | | CACCACCACCCGTTTACAAATACAAGTCTCCACCACCTCC |
| | | GGTCTACAAGTACAAATCTCCACCACCACCCGTCTACAAG |
| | | TACAAGTCCCCACCACCTCCAGTCTACAAGTACAAGTCTC |
| | | CACCACCACCCGTCTATAAGTACAAGTCTCCACCACCTCC |
| | | AGTCTACAAGTACAAGTCTCCACCACCACCTATTTACAAG |
| | | TACAAATCTCCTCCACCACCAGTTTACAAATACAAGTCAC |
| | | CACCACCACCCATTTACAAATACAAATCACCCCCACCTCC |
| | | CGTTCACAAGTCTCCAGCCCCTTACTATTACACTTCTCCA |
| | | CCTCCTCCTAGCCACTACTAGTTTCACAATCTCGAACGAG |
| | | GTCAAGAGGAATAATTAA |
| SEQ | Solyc05g005200.3 | ATGGGATCGGAGGGGCCAAAATCTGTGACCATTCATGTCA |
| ID | | CGGGGTTCAAGAAATTTCACGGTGTAGCTCAGAATCCTAC |
| NO: | | GGAGACGGCAGTTAGCAACTTGAAATATTATGTTGAAAAG |
| 183 | | AAAGGATTACCTGCTGGTGTGACCTTGGGTAGCTGTACTG |
| | | TTCTTGAAACAGCTGGAGAGGGTGGACTTCCAACATTGTT |
| | | GAAGGTTATAGAGGCGTCCTCTGCTAGTAACAGTTCAAAT |
| | | AATGGACAAGTCATATGGCTTCACCTTGGGGTAAACAGTG |
| | | GAGCAACAAAGTTTGCAATTGAGAGGCTGGCAGTTAATGA |
| | | AGCCACATTTCGTTGTGCAGATGAGCTTGGTTGGCAACCT |
| | | CAGCGAGTGCCTATTGTTCCAGAAGATGGAGGAATAAATC |
| | | AAATAAGAAAGACATGTTGCCCTACCGAGTCAATCCTGGA |
| | | GTTGTTAAAGAAGAAAGGGTTTGATGTGACGTTGTCGGAC |
| | | GATGCAGGTCGTTTTGTGTGCAATTATGTGTACTATCATT |
| | | CTCTCCGATTTGCAGAGGAAAAAGGTCACAAGTGTCTGTT |
| | | TGTCCATGTTCCTACCTTCCATAGAGTTCATCAAGAGAAA |
| | | CAAATGGAATTTGTTGCTGCTCTGCTGGAGGCTATTGCAT |
| | | CAACTTGTTGA |
| SEQ | Solyc05g015630.3 | ATGGACCCTAACGACCTAGCGAAGGCATTTGTGGAGCACT |
| ID | | ACTACTCCACCTTCGATAGCAACAGGGCTGCGCTTGGAAA |
| NO: | | TCTGTACCAAGAAGGTTCCATGTTGACATTTGAAGGCATT |
| 184 | | AAAACTCAAGGTTCACAAAATATTGTCGCTAAACTTACTA |
| | | GCCTTCCCTTTCAGCAGTGTCATCATACCATCACTACCAT |
| | | TGATTGTCAGCATTCTGGTCCTGCTGGTGGCATGATTGTT |
| | | GTCGTCTCCGGTAATCTCCAGCTAGCCGGCGAACAGCATG |
| | | CTCTTAAGTTTGGCCAGATGTTTCATCTGATGCCAACGCC |
| | | ACAAGGAAGCTTCTACGTCTTCAATGACATTTTCCGGTTG |
| | | AATTATGGCTGA |
| SEQ | Solyc05g050800.3 | ATGGCGGCGAATCAAAATGTAATTGTAATGAGACACGGTG |
| ID | | ACCGGTTGGATAACTTTGAGGAATTGTGGGTGATGAAAGC |
| NO: | | GGAAAGACCGTGGGATCCACCTTTGCATCAGGATGGTAAG |
| 185 | | ATCCGGGCATTTTGTACGGGTCAAAAGATCCGATCCAGTA |
| | | TTGATTTCCCGATCCATCGGGTTTTCGTTTCTCCGTTTCT |
| | | CCGATGTGTGCAGACTGCTGCAGAGGTCGTACGTGCTCTT |
| | | TGTGACGTTGCTGATCACAACGGCGAGTCTAATGTCTTGT |
| | | CATCCAATTCCGATTCAGTCATTATTGATCCATCTAAAGT |
| | | CAAAGTATCTATTGAATATGGTTTGTGTGAAATGCTGAAT |
| | | TTAGTAGCTGTAAGAGCTTCTGCAGCTCCTAAGGATGGGG |
| | | ATTTCAAATTCAGCATCTCACAATATGAATCTGAGTTGCC |
| | | TGCTGGAACTGTAGATAACACTGTTGAACCTGTGTATAAG |
| | | AAGCTGCCAGAGTGGGAAGAGTCGTTGGAAAGTGCAAGGG |
| | | CTAGATATGTGAAGGTAGTTAAGGCCCTTGCTGATAAGTA |
| | | TCCTTCTGAGAACCTGTTGTTAGTCACACATGGTGAAGGA |
| | | GTAGGTTCCATCTTTACTGAGCTTAACAAGGATTCAACTG |
| | | TATTAGAGGTAGCGTATTGTGGACATCTGCACGTAAAGAG |
| | | AAGTATCCAATCCGGAGAGAAACAACCATTTACTGCTGGA |
| | | GAGTTTGTATACGAGAAACAAACGGGTATAATATCTGCTG |
| | | CTAAATAA |
| SEQ | Solyc05g056250.3 | ATGGAGTCGGAGAAACCCCAAAATCCATCTCCAATGGAAC |
| ID | | CACAAGAAGAAGGCAGCAACAAAATCTCCAAAAAAGAAGC |
| NO: | | CGCCAAGCTGGAGCGTCAGCGCCGCCGTCAAGAGGCAGCC |
| 186 | | GCCGCCGCCGCCGCCACTGCTGTCTCCACTGTCTCCATTG |
| | | AAGAATCTGATCCACTTTCCGGAAACTACGGTGACGTAGA |
| | | GCTGAACGATCTGCAATCTAAGGCCGTGACGGGTCGAAAG |
| | | TGGACTGAAGTTGGGTCCATCGCGCCAGAAATGAAGGACC |
| | | GGGTGGTCTTGGTTCGGGGGAGGGTACAGACGATTCGTCC |
| | | TGTTGGGAAAAAGATAGCTTTTGTGGTTGTGAGGGAACGA |
| | | GGATTTACGGTGCAGTGTGTATTGACTGTGAAGCCTGAAT |
| | | TGGTTAGTTCACAAATGGTGAAATATGCTACTAGTTTGAG |
| | | CAAAGAGTCCATTATTGATGTTGAAGGTGTTGTTACAATA |
| | | CCAGAGAAGCCAATTACTGGTGCCACTCAACAACAGGTGG |
| | | AAGTTCAAATAAGCAAACTTTATTGTGTCAACAGGGCTGT |
| | | GCCCACTCTACCGATTAATATTGAGGATGCTGCTAGAAGC |
| | | GAGGTGGAGATAGAACAAGCTTTAGAGAAAGGAGAGCAGC |
| | | TTGTTCGTGTAAATCAGGATACTCGCTTAAACTTCAGAAT |
| | | ACTAGACATGCGCACACCTGCAAATCAAGGAATCTTCCGC |
| | | ATTCAATGTCAAATCGAAAATATATTCAGGCAGTTTTTGC |
| | | TCTCTGAAGGATTTGTAGGAATCCATACTCCAAAATTGAT |
| | | TGGAGGGTCTAGTGAAGGAGGTTCAGCTGTTTTCCGATTA |
| | | GATTATAAAGGTCAACCTGCATGTCTGGCGCAGTCACCTC |
| | | AACTTCACAAACAAATGGCAATATGTGGTGATTTTGGCCG |
| | | TGTATTTGAAGTAGGCCCCGTCTTTAGGGCAGAAGATTCT |
| | | TTTACACATAGACATCTGTGCGAGTTTACAGGTCTTGATG |
| | | TTGAAATGGAGATCAAGGAACATTATTCAGAGGTCATGGA |
| | | TATTGTTGACCGCTTATTTGTGGCCATGTTTGATAGCTTG |
| | | AATGAGAAATGCAAAAAGGAACTTGAAGCCGTTGGAAAGC |
| | | AATACCCTTTTGAACCTCTGAAGTATTTGCGAAAGACCTT |
| | | ACGACTTACATTTGCTGAGGGGATTCAAATGCTAAAGGAA |
| | | GCTGGTGTTGAAGTTGATCCTCTTGGGGACCTAAATACAG |
| | | AATCAGAAAGAAAGCTTGGGCAGCTTGTTGCGGAGAAGTA |
| | | TGGAACTGAATTCTATATACTTCACAAGTACCCACAGGCT |
| | | GTTCGTCCATTCTATACCATGCCTTGTCATGACAATCCAT |
| | | CTTACAGCAATTCTTTTGATGTCTTTATCAGAGGGGAGGA |
| | | AATTATCTCAGGAGCTCAGCGTGTCCATGTTCCTGAACTC |
| | | TTGGAAAAACGTGCCGGAGAATGTGGAATTGATGTGAAGA |
| | | CAATATCAACATACATTGATTCTTTCCGGTATGGTGCACC |
| | | TCCCCATGGTGGATTTGGAGTCGGATTGGAGCGTGTGGTG |
| | | ATGCTTTTCTGTGCTCTCAATAACATTCGCAAGACATCAC |
| | | TTTTCCCTCGCGACCCACAAAGGATTGTCCCATGA |
| SEQ | Solyc05g056340.3 | ATGAGTGGTACTCCGAACAAAAGACCTCACGAGGATGGTG |
| ID | | GAAATGGTGGGAGTAGTAACCATAGTTACTCTTCTGCTCC |
| NO: | | AAAATACTCACATGATGACTCTGGTGCATTTCCCAAGGTG |
| 187 | | ATGAGCTCAGGAACACCTGAATATCATGCCTCCTTTGATG |
| | | TGGGCCAGAATGCTCGGATGCCGAAGATTCAACGGACTGA |
| | | ATCTTCACGAGATGCAGATAGAAGATCTCCTGTGCTTCCA |
| | | ATGTACCGTGTCTCATCATGTCCAGTTGTTTCACATCCTG |
| | | ATCATTCTGTTGCTTCAGAAAATAGGTTGGAGCCCAAGGA |
| | | AGTTAACAAGGACGTCAAGGTTGAGAATCGTGATGCCAAA |
| | | AGTGAAATAAGGGAGTTGTACCAAGGGACTAAATCTGACA |
| | | AGGATGATAGATTTGAGAACAGAGCTGATGATGGTAAGGA |
| | | CATTAAAAATAGTAGGGATACTTACCCTGAATACAAGGGA |
| | | GATGTGAAGACAGATAAGGACAGGTTTAGCGGAGTGAGTT |
| | | GGAAAGATCCGAAAGAACAGACCAGGGGAAAAAGATATCC |
| | | TGATCTCCCTGTTCCTGTCGGGAACATGGATCCATGGCAT |
| | | GCGTCAAGAACCCATGGTGCTGCTGAGATAGGAAAAGAAG |
| | | TCTCAAATTCTGAGAACAGGGATTTTGCTAAAGTGCGTGA |
| | | AGCCGTTGCTGAAAATAAGATGGATTTGAAAGGTGACGAT |
| | | AAATACAAAGATAAAGAGAGAAAAAGGAAAGAAGGGAAGC |
| | | ACCGGGAATGGGGAGAAAGGGATAAAGAGAGAAATGATTG |
| | | TCGGAACAATTTACAACTAGGGAATAGCACTTCTGATAAC |
| | | AAGGAATTGCTTAAAGAGGAAAGGGAATCTGAGCGGTGGG |
| | | AGAAGGAAAGAAATGATCTTTCGAAGGATAAGGACAGACC |
| | | AAAGGACTGGGAAAAGGACCATGCAAAGAGGGAAGTGTGG |
| | | AATGGAGTGGAGAGGGAGGTTTTGCAGAGTGAGAAAGAAG |
| | | TGATTGATGTTCCTGGAAAAACAAACGAGCCGGAAAACTC |
| | | AACAGTGGAGCAGAAGAAACAGAAAGATCATGATAACTGG |
| | | AAAAATACTGACAGGGATGGAAGTGAGAGGAGAAAGGAAA |
| | | GAGATACTGATTTGGAAGGAGAGAGGCCTGAGAAACGTGT |
| | | CAGGTGTCATGATAAAGAACCAGAGGAAGGGGACCTGGAT |
| | | ACTGAAGGAGGAGGAGAAAGGGAAAGAGAAGCTTTTAATT |
| | | ATGGAGTTCAGCAGCGCAAGAGAATGTCGCGGCCAAGAGG |
| | | GAGCCCCATGGCCAATCGCGATCCTCGTTTTAGGTCGCAC |
| | | ACTCATGAAAATGAAGGATCTCAAGTGAAGCATGATGTAT |
| | | CTGCTGTCAATTACAGAGTTGGTGAGTGTATGCCAGAACT |
| | | GATTAAATTATGGAAGGAATATGAATCATCCAAAGCAGAT |
| | | GAAGCATCTGATAGCTCTCCAAGTGATCCTACTCTAGAAA |
| | | TTAGGATTCCAGCTGAACACGTATCAGCTACAAATCGGCA |
| | | GGTGAGAGGTGGCCAACTATGGGGAACAGATATATACACC |
| | | AATGACTCGGATCTTGTCGCAGTTCTTATGCACACAGGTT |
| | | ACTGTCGTACAACTGCGTCTCCTCTTTTGCCTACTATTAC |
| | | GGAGTTACGTGCTACTATCAGGGTACTACCTCCACAAAAT |
| | | TGCTACATATCTACTCTGAGGAACAATGTGCGATCACGTG |
| | | CGTGGGGAGCTGCAGTTGGCTGCAGCTATCGTATTGAGCG |
| | | GTGCTCTGTTGTGAAGAAAGGAGGTGGAACAATCGATCTT |
| | | GAACCTTGTCTAACACATTCCTCAACCTTGGAGCCTACTC |
| | | TTGCTCCGGTGGCGGTAGAGCGCACTATGACCACTCGAGC |
| | | TGCAGCTTCGAATGCACTACGACAACAGAGGTTTGTACGT |
| | | GAAGTGACAATTCAGTTCAACTTATGCAATGAGCCTTGGC |
| | | TCAAATACAGTATCAGTGTTGTTGCTGACAAGGGTCTAAA |
| | | AAAGGCCCTTTTTACATCTTCACGCCTGAAGAAGGGAGAA |
| | | GTTCTTTACTTGGAAACTCATTCTAAGAGGTATGAGCTCT |
| | | GTTTTAGTGGTGAAAAGATGGTTAAGGCTACAACTTCTCT |
| | | GATGCATGAAATGGATGTTGACAAACCTCAAAGTCACAAT |
| | | TTACACATGGCAAACGGAGAAAAAAATGGAGTGAATGGTG |
| | | AGAATACGATGGTAGATATGTTCCGACTGTCTCGTTGTAA |
| | | GAAGCCCCTGCCTCAGAAACTAATGCAATCAGTTGGAATT |
| | | CCTTTGCCCCTTGAACATGTTGAGGTTTTGGAGGAGAATC |
| | | TGGAGTGGGAAAACATTCAATGGTCACAAACTGGTGTTTG |
| | | GATTGCTGGAAAAGAATATCCTCTTACTAGAGCGCATTTT |
| | | CTTTCCCCAAATTAG |
| SEQ | Solyc06g008340.4 | TTCTTCACTTCAAATTACTCAACCAAAACGATGTCGTTTG |
| ID | | ATGAAGATGAAGAATCCTTCGAGCACACGCTCCTAGTAGT |
| NO: | | GCGTGAAGTTAACGTCTTCAAAATTCCTCCTCGGCCAACC |
| 188 | | TCCGGCGGCTACAAATGCGGCGAGTGGCTTCAAACCGACA |
| | | AGATCTGGTCGGGTAGGCTCCGGGTCGTGTCATGTAAGGA |
| | | TCGGTGCGAGATCCGATTAGAGGATCCCAATTCCGGCGAG |
| | | TTGTTCGCAGCTTGTTTCGTGCCGCCGGGACAGAGGGAGA |
| | | ATTCGGTGGAATCGGTGCTTGATTCGTCGAGGTACTTTGT |
| | | GTTGAAGATCGAAGATGGTACAGGGAAGCATGCGTTTATT |
| | | GGGTTAGGGTTTAGCGAGAGGAATGAAGCGTTTGATTTCA |
| | | ATGTAGCGTTATCGGATCATGAGAAGTATGTGAAGAGAGA |
| | | TATTGAGAAAGATGGTGATGAAGCTGAGACGAGTAGTGAT |
| | | GGTCATATTGATATTCATCCTGCTGTTAACCATCGATTAA |
| | | AGGAAGGTGAAACCATCAGGATAAACGTGAAGAACAAATC |
| | | ATCTAGTGGAATGGGCATGCTTTCTGCTGCTGTTCAATCA |
| | | AAGGCCATTACCATTGCTCCACCCCCTAGCGGAGCTAACA |
| | | AGATCAGGTCTCCTCTTCCACCCCCTCCGAATGATCCAGC |
| | | TATTTCTCGGAAGACTTCTACCACTGCTAGTATTGCACTC |
| | | AAGGGGCCAAATGGAAGCTCAATGCAGTCCAGTGATCCAA |
| | | TTTCCGATCTCTCTCAACTTGAGAGGAGCCTTCCTTCAGC |
| | | CACTGGATCTGGTTCGAGCAAGAAGACCACAGCAGCTGGA |
| | | TGGGCAGCATTTTGA |
| SEQ | Solyc06g050770.3 | ATGGGGGATCAAATAGCAAGAGGTGAGGATTTCGAGAGCA |
| ID | | AGGCTGAGAAGAAACTCGGCGGATGGGCCATTTTCGGTTC |
| NO: | | CAAATACGAAGACGCAGCTGATCTGTTCGATAAAGCTGGC |
| 189 | | AATTGCTTTAAACTCGCCAAATCATGGGATAAAGCTGGAG |
| | | CAGTATATGTTAAATTGGCTAATTGTCATCTTAAGTTGGA |
| | | TAGCAAACATGAGGCTGCAAATGCTTATGCAGATGCTGCT |
| | | CATTGCTACAAGAAGTCAAATATTAAAGAGGCAGTATCAT |
| | | GTTTAGAGCAGGCAGTGAACTTATTTCTGGACATTGGGAG |
| | | GCTAAATATGTCTGCAAGATATTACAAGGAAATTGCTGAA |
| | | TTATATGAGCAAGAGCAAAATTTGGATCAGGCTATTAATT |
| | | ACTATGAGAAGGCAGCTGATCTTTTCCAGAGTGAAGATGT |
| | | AACTACAACTGCAAATCAATGCAAACAGAAAGTTGCACAA |
| | | TTCTCTGCTCAAATCGAGAAATATCCGAGAGCGATCGAGA |
| | | TCTTTGAGGAGATTGCACGTCACTCAGTCAATAACAACTT |
| | | GTTGAAGTATGGAGTTAGAGGACATCTTCTTAATGCTGGT |
| | | ATTTGCCAACTTTGTAAAGGTGATGTTGTTGCAATCAACA |
| | | ATGCGTTGGAGCGATATCAGGAGCTGGATCCAACATTTTC |
| | | AGGGACACGTGAATGCAAATTGCTAGTGGATTTGGCAGCT |
| | | GCAATTGATGAGGAAGATGTTGCAAAGTTCACTGATGCTG |
| | | TCAAAGAGTACGATAGCATGACACAACTGGATGCTTGGAG |
| | | GACAACACTACTTCTAAGAGTCAAGGAAACATTGAAGGCG |
| | | AAGGAGCTTGAGGAAGATGATCTTACTTAA |
| SEQ | Solyc06g053510.3 | ATGGAGGTCATAATGAACAATCACAACAATGGCACCATCA |
| ID | | ACACCAACACCAAAATTATTCACAAAAATGGTAGCATTTG |
| NO: | | CAATGGCAATGGCAATGTTAATGGCAACTCCCACACCCAT |
| 190 | | AACAATGAAAATAAGCTTGTAGAGTTCACTAACTCTATCA |
| | | AACCGGGTTGGTTTTCTGAGTTCAGCGCACTCTGGCCAGG |
| | | TGAAGCATTTTCACTTAAAATTGAAAAATTACTATTCCAA |
| | | GGAAAGTCTGATTATCAAGATGTTATGCTCTTTGAGTCAG |
| | | CAACGTATGGGAAGGTATTAACATTGGATGGGGCAATTCA |
| | | ACACACAGAGAATGGTGGATTTCCCTACACTGAGATGATT |
| | | GTTCATCTACCACTTGGTTCCATTCCATCCCCTAAAAAGG |
| | | TGTTAATCATCGGTGGAGGGATTGGTTTCACGTTGTTTGA |
| | | GGTCTCTCGCTATCCAAGTATTGAAAAAATAGACATAGTT |
| | | GAGATCGATGACGTGGTTGTAGACGTATCTAGAAAGTTTT |
| | | TCCCATACCTAGCAGCAGGATTTGATGATCCAAGAGTCAG |
| | | CCTTATTATTGGCGATGGAGCTGCATTTGTGAAAGCTGCT |
| | | CAACCTGGATACTATGATGCAATTATTGTTGACTCTTCTG |
| | | ATCCTATTGGTCCAGCAAAAGACTTGTTTGAAAGGCCATT |
| | | TTTCGAAGCAGTGGCAAAAGCCCTAAGGCCAGGAGGAGTA |
| | | GTGTGTACACAGGCAGAAAGTATTTGGCTTCACATGCATC |
| | | TAATTAAAAAAATTATTGCAAATTGTCGACAGGTCTTTAA |
| | | GGGTTCTGTCAATTATGCATGGACTACCGTTCCTACTTAC |
| | | CCTACTGGTGTGATTGGTTACATGCTTTGCTCTACGGAGG |
| | | GACCAGAAGTTAATTTCAAGAATCCAGTGAACTCTATTGA |
| | | CAAATATGTCAAATCCAAGGGACCTTTGAAGTTCTATAAC |
| | | TCTGATATTCATAAATCAGCTTTTATTTTGCCGTCTTTTG |
| | | CAAGGGATTTGATTGAATCTTGA |
| SEQ | Solyc06g065100.3 | ATGGGACGTTCACCTTGTTGTGAAAAAGCACATACAAATA |
| ID | | AAGGAGCATGGACTAAAGAAGAAGACCAACGCCTCATCAA |
| NO: | | TTATATACGTGCTCATGGGGAAGGTTGCTGGCGTTCTCTT |
| 191 | | CCTAAAGCTGCAGGATTGTCAAGATGTGGAAAGAGTTGCA |
| | | GATTGAGATGGATAAATTATTTAAGGCCTGATCTCAAAAG |
| | | AGGGAATTTTACAGAAGAAGAAGATGAATTGATAATCAAA |
| | | CTTCATAGTTTGCTTGGAAACAAATGGTCAGTTATAGCCG |
| | | GAAGATTACCTGGAAGAACGGATAACGAAATCAAGAACTA |
| | | TTGGAATACACATATTAAACGAAAACTCATTAGTCGTGGC |
| | | ATTGATCCTCAAACTCACCGTCCACTCAACAACAACGCCA |
| | | CTAACTCCCACACCACCACCAATATCACCACCGCAGTCAC |
| | | AACCAAAAACATCAACTTGGATTTCACAAACGTTGACCAA |
| | | AAACAACCCAATATTATGATTGCCACGTCATCGTCATATG |
| | | ATGAAACAAAATGCAACAGTGGTACAACTGAGGAAACAAA |
| | | GCCACTAGAAATTATTATTCCAAAAATACCCTCACAAGTT |
| | | ATGATTAATCTTGAACTTTCAATTGGGTTGCCGTTACATA |
| | | CTGATCATATTTCTTCACCAGAGTCAACGGCCTCATACAA |
| | | CTTCTTGACCACCGTAGCACCGCCGCCAACTGCGGCGGTA |
| | | CCGGCGGCGGAGATGATGGCGAAGACTGTTTGTTTGTGTT |
| | | GGCAAATTGGATATCATGGTGGTGGCGGTCAGTGGTGTGG |
| | | TAAATGCAAAAACACAAATGGATTTTACAGATATTGCTGA |
| SEQ | Solyc06g072120.3 | ATGGCGACGCAAGATGTAAGGACTCTTTCGACTAAAGAAG |
| ID | | CTGACATTCAGATGATGTTGGCTGCTGAAGTTCACCTCGG |
| NO: | | TACTAAGAATTGTGACTTCCAAATGGAGCGATACGCTTTC |
| 192 | | AAGCGCCGTAACGATGGTATCTACATCATCAACCTTGGAA |
| | | AGACATGGGAAAAGCTTCAAATGGCTGCCAGGGTGATTGT |
| | | TGCTATTGAGAATCCTCAGGACATCATTGTTCAATCTGCC |
| | | AGGCCTTATGGACAGAGAGCTGTCTTGAAATTTGCTCAAT |
| | | ATACTGGTGCACATGCTATTGCTGGACGTCACACCCCTGG |
| | | AACTTTTACCAATCAGCTTCAGACCTCATACAGTGAGCCA |
| | | CGACTCTTGATTCTGACTGATCCAAGAACTGACCATCAGC |
| | | CAATCAAGGAAGCTGCACTTGGAAACATCCCAACTATTGC |
| | | TTTCTGTGACACTGATTCTCCAATGCGTTATGTTGACATT |
| | | GGTATCCCTGCCAATAACAAAGGAAAGCACAGCATTGGTG |
| | | TTCTTTTCTGGATCTTAGCAAGGATGGTACTCCAGATGCG |
| | | TGGGGCCATTAATCAAGGACCTAAATGGGATGTTATGGTT |
| | | GATCTCTTCTTTTACAGAGAGCCTGAAGAGGCAAAGGAGC |
| | | AAGAAGAGGAAGTGCCAGCAATTGCAGATTATGCAGACTA |
| | | CTCTGCGAGTGCTGCCCTTGGTGGTGACTGGACTAGTAGC |
| | | CAAATCCCTGAGGCGCAATGGACTGCAGATGCTGCAGCAC |
| | | CTGCAGTAGGTGGTGGTTGGGCTGGAGATGGAGCAGCTGA |
| | | TGGCGGATGGGATGCAGCAGCAGCCCCTGCACCAGTTCCT |
| | | CTACCAGTACCAGATGTTGCCCCTACCTCTGGTGCTACAG |
| | | GCTGGGAATGA |
| SEQ | Solyc06g073150.3 | ATGGACTCTCTGAATTCTTACGCATCGTCGGCGGCGATGG |
| ID | | CGCAGCAAACCTGGGAGTTAGAGAACAACATCGTAACAAT |
| NO: | | CGACGCGCCGTCGGGGTCGAAACCGGAAAACTCCGCGTCG |
| 193 | | GACGCTATATTCCACTACGACGATGCGGCACAGACGAAGT |
| | | TTCAGCGGGAGAAGCCGTGGGCGAGTGACCCTCACTACTT |
| | | CAAGCGTGTGAAGATCTCTGCTCTTGCTCTTCTCAAGATG |
| | | GTTGTTCACGCGCGTTCCGGAGGTACAATTGAGGTCATGG |
| | | GACTCATGCAGGGTAAGACTGACGGCGATGCCATCATTGT |
| | | AATGGACGCTTTTGCCCTCCCCGTTGAAGGCACTGAAACT |
| | | AGGGTTAATGCTCAAGCTGATGCGTATGAATATATGGTCG |
| | | AATATTCACAGACCAACAAGCAGGCTGGTCGTTTGGAAAA |
| | | TGTGGTCGGGTGGTACCATTCCCATCCTGGTTATGGATGC |
| | | TGGCTCTCTGGCATTGATGTATCTACCCAAATGCTCAACC |
| | | AGCAATATCAGGAACCCTTTCTTGCAGTTGTTATTGATCC |
| | | AACAAGAACTGTTTCTGCTGGAAAAGTTGAGATTGGTGCC |
| | | TTTCGAACATATCCTGAAGGATATAAACCTCCAGATGACC |
| | | CCATCTCGGAGTACCAGACCATTCCTTTGAACAAAATCGA |
| | | AGACTTTGGAGTACATTGCAAGCAGTATTATTCATTGGAT |
| | | ATTACATATTTTAAGTCTTCTCTCGATTGTCGTCTCTTGG |
| | | ACCTACTATGGAACAAGTATTGGGTGAACACACTTTCATC |
| | | ATCTCCTTTGCTTGGAAATGGAGACTATGTTGCTGGACAG |
| | | ATATCTGATCTTGCTGAAAAGTTGGAGCATGCTGAAAATC |
| | | AGCTGTCTAATTCACGTTATGCATCCTTAATGGCACCCCC |
| | | TCAAAGGAAGAAGGAGGAAGAATCTCAGCTTGCTAAGATT |
| | | ACACGTGATAGTGCTAAGATTACTGTCGAACAAGTTCATG |
| | | GCTTAATGTCGCAGGTAATTAAAGACATTCTTTTCAATAG |
| | | TGTTGGTAAGTCAAGCAAGTCACAGACGGAATCCTCAGGT |
| | | CCCGAGCCCATGATCGAAAGCTGA |
| SEQ | Solyc06g076410.4 | ATGGCATCCATGATTGAATCTGCTTGGACGTATCTGATTA |
| ID | | CAAATTTCAATGACTTCCAATTGGCTTGTCTTGGAAGTTT |
| NO: | | CTTTCTTCATGAAAGTGTCTTCTTCTTGTCTGGACTTCCT |
| 194 | | TTCATTCTTCTAGAAAGGGCAGGATGGTTGAGCAAATATA |
| | | AAATTCAGACAAAAAATAATACTCCTGCAGCTCAGGAGAA |
| | | ATGTATCACTCGCCTGTTGCTGTATCATTTTTGTGTTAAT |
| | | CTCCCGCTTATGATGGCATCCTATCCAGTCTTCAAATTAA |
| | | TGGGGATGCGAAATACTCTTCCGTTGCCGTCCTGGAAAGT |
| | | GATCTCTACGCAAGTATTATTCTACTTTATCCTGGAGGAT |
| | | TTTGTATTCTATTGGGGACACAGGATTTTACATACTAAAT |
| | | GGCTCTACAAGCATGTCCACAGTGTCCATCACGAGTATGC |
| | | AACACCATTTGGATTGACCTCTGAATATGCTCACCCTGCT |
| | | GAAATTCTTTTTCTTGGATTTGCTACAATAGTGGGTCCAA |
| | | TCATTACTGGGCCCCATTTGATAACATTATGGTTGTGGAT |
| | | GATAGTTAGAGTCCTTGAGACAGTTGAAGCACATTGTGGT |
| | | TACCATTTCCCATGGAGCCTATCAAACTTCTTGCCATTAT |
| | | ATGGGGGGGCTGATTTTCATGATTATCATCACCGACTGCT |
| | | CTACACAAAGTCTGGAAATTATTCTTCAACATTTGTTTAC |
| | | ATGGACTGGTTATTTGGTACTGACAAGGGTTACAGAAGAT |
| | | TGAAGGTGCTGAAGAGTGATGCATGTGAAGCTGAGGGTAA |
| | | AGAAATGTAGAGATGTGAAAGGGTAAGTTTTGGCTGGTCT |
| | | GGCATTGCAAGACGGGCTTATGTACAAGAGCGTATAGGTG |
| | | AAGGTTTCTGTTGTTGA |
| SEQ | Solyc06g082380.3 | ATGGAAACCACTGCTACGCCGGAGGAGTGGCCGCCGGACG |
| ID | | GGCAATTTCCATTTGATCTTTTTGTATCGAAGAAGTATAA |
| NO: | | GTCCTTCTGGGATGCTGGAAACATCCAATTTACCGATGCT |
| 195 | | GTTGGTAATTTGTTCTTCAGCGTTGACCGTCGTCAACCTC |
| | | AGAATTCGGCAGCCCAGACCCACCCTCAAAAACTAATTCT |
| | | CGATGCATCGGACAATACCCTGATTCGCTTAGTTCGATTA |
| | | ACTAAAGGATCATGGCAAGGCTTTATGGTAAGTGATAATG |
| | | AAGAAAAGGCGTTGATGTTCAGTGTGAATAAGACACTAAA |
| | | TACATTCACTAAACTAGAGTTTGACATATTCCTTGGTGAT |
| | | GGACATGTTGAAGGCGCAGAGGCTGATCTTAAGATGAAAG |
| | | GTTCTGCCTTCAAGAGATCATGCACCATCTACAAAGGCAA |
| | | TTCCATAGTAGCTGAGACTAGCCTTATGTACACACTTGGG |
| | | TTCAGGAAGCATTTCATTCCAAGGAACAGATTTCGAGTAA |
| | | CCATATTTCCCGGTTTGACTGAGCTTAGCCTTGTTGTGGC |
| | | TTTGGTTGTAGTATTTTTTGACAAACAGAAATTTTGGCTA |
| | | TAA |
| SEQ | Solyc06g082670.3 | ATGGGGAGAAGACCTGCAAGATGTTATCGTCAGATTAAGA |
| ID | | ACAAGCCTTACCCAAAATCAAGGTTTTGCCGTGGTGTCCC |
| NO: | | TGATCCAAAGATCAGGATTTATGATGTGGGCATGAAGAAA |
| 196 | | AAGGGAGTTGATGAGTTCCCTTTCTGTGTGCATTTGGTCA |
| | | GTTGGGAGAAGGAGAATGTCTCAAGTGAGGCACTTGAAGC |
| | | TGCCCGTATTGCTTGCAACAAGTACATGACCAAGTCTGCC |
| | | GGGAAAGATGCTTTTCACCTAAGGGTCAGGGTTCATCCAT |
| | | TCCATGTTTTGCGAATTAACAAGATGTTGTCGTGTGCTGG |
| | | AGCTGATAGACTCCAAACTGGTATGAGGGGAGCTTTTGGT |
| | | AAGCCCCAGGGTGTTTGTGCTCGTGTTGCAATTGGTCAGG |
| | | TTCTTCTATCTGTTCGCTGCAAAGATGGAAACAGTAACCA |
| | | TGCCCAAGAGGCTCTGCGCCGTGCCAAGTTCAAGTTTCCT |
| | | GGACGTCAAAAGATCATTGTCAGCAGGAAGTGGGGATTCA |
| | | CCAAGTTCAGCCGAACTGATTATCTGAGATACAAGTCAGA |
| | | GAACCGTATTCTCCCAGATGGTGTGAATGCAAAGCTTCTT |
| | | GGCAACCATGGTCGACTTGCTGCACGTCAACCTGGAAGGG |
| | | CCTTCCTTACATCTGCTTAG |
| SEQ | Solyc06g083210.3 | ATGGAAAGATATAGAATATTGAAGGAGCTTGGAGATGGAA |
| ID | | CATGTGGTAATGTATATAAGGCCATTAATACCGAAACATC |
| NO: | | TGAAATTGTTGCAGTAAAGAAAATGAAGAGGAAGTTCTAT |
| 197 | | TTTTGGGAAGAATGCATTAATCTACGTGAAATAAAGTCCC |
| | | TCCGTAAATTGAATCATCCTAACATCATCAAACTGAAAGA |
| | | GATTGTCAGGGAAAACAATGAGCTTTTCTTCATACTTGAG |
| | | TACATGGAATGTAACTTGTACCAACTAATGAAAGATCGGC |
| | | AAAGACCTTTCCTAGAGGAAGAGATTCGAGGATTGATGTC |
| | | TCAGGTGTTGCAAGGACTTACCCATATGCATAAAAATGGT |
| | | TACTTTCATCGGGACTTAAAACCTGAGAATTTACTGGTGA |
| | | CGAATGACGTAATTAAAATTGCTGACTTTGGGTTGGCTCG |
| | | TGAAGTGTCTTCATCACCTCCTTTCACTGACTATGTTTCA |
| | | ACTCGTTGGTACCGAGCACCAGAAGTTTTGTTGCAATCTT |
| | | CATCATACACACCTTCTATCGATATGTGGGCAGTTGGTGC |
| | | GGTACTTGCTGAGCTTTTCACTCTTTGCCCAATATTCCCT |
| | | GGTGAAAGTGAAATTGATCAGTTGTATAAGATATGCTGTG |
| | | TTCTTGGACCGCCAGATTGGACTAGCTTCCCAGAACTGAG |
| | | AAGTGCTTCACGGTTGTTTGACATTAATTGTTTTGATATT |
| | | ACACCAGCCAATCTGTCTGATTTCATTCCAAATGCTAGCT |
| | | TGGAAGCTATCGACTTGATCAAGCAACTCTGCTCGTGGGA |
| | | CCCATTGAGAAGACCAACTGCTGACCAATGCTTACAGCAC |
| | | CCATTTTTTCATGTTGATATGTCAATTCCTCGTCCTCTAG |
| | | AGGATCCACTGCAGATGAATTTAAGTAGTGTTGGACCTGA |
| | | GCCTAATCTTGAGTTGAACCTGTGGGATTTTGGGACAGAA |
| | | AAAGATGACTGCTATCTTGGTTTGACTTTGGCTGTGAACC |
| | | CAACTCCCTCTTGTCTAGATTTATTTCCAGCAAAGATGGC |
| | | AAGTAAAAGTCAAGGTGCAGGAACGGATATGTTCTGCTCT |
| | | GGTTTCCAAGATCACTCGCAACACTCAGTTTTCTGGTCAT |
| | | TGTTTCCTACTGATCGCCATCAGATATCTACTCCGGTGGA |
| | | CTCCTCATTGTCATTATCATTCAGCACGATTCCACAGTCA |
| | | ACTATTGGAGCTCCACAATCAACTAGTTTTGGCATGACAT |
| | | CTCTGCAGAGTAACTTCTTGGAGCGCCCCTTTCTGGCTAT |
| | | GTCATCCTCCTTTCAGCAGGGACGTTGCCTTTGA |
| SEQ | Solyc06g083300.4 | ATGGCAGCGGAAGCAGCAGTAGCAGTAGCGGCAGTAGCAC |
| ID | | CAGTTTCTGCTCAAGTTGTTGCGAATGCTTTTGTGCAGCA |
| NO: | | ATACTATCATATATTGCATCATTCGCCGGGGCTTGTATTC |
| 198 | | AGGTTTTACCAGGATATAAGCAAGCTTGGACGTCCTGAAG |
| | | AGGATGGATCCATGGGTATCACTACGACAATGCAAGCAAT |
| | | AAATGATAAGATACTCTCACTCAACTATGGAGATCTCAGA |
| | | GCAGAGATCAAGTCTGTGGATGCCCAAGAATCCTTTAATG |
| | | GCGGGGTGCATGTCCTTGTAACTGGATATTTGACTGGGAA |
| | | AGACAACTTGGTCCGGAATTTCTGTCAAACTTTCTTCCTA |
| | | GCACCACAAGATAGGGGGTACTTTGTCTTGAATGATATGT |
| | | TTCGATATGTAGAGACTGCCAGTCAGCATGATACAGTCCA |
| | | GGTTCCAGTAACTGATGCTGTGGCTCCTGTCACACCAGAG |
| | | CAAATTGTTGCAGATCCACCTACAGCACCACAGAATCACA |
| | | TTCCTGAGCAAAGCACTCCACCAGCAGAGGAAGCTGACGT |
| | | GGGAGAAGTTTACAACCCATCTGAGAATGGTGATGTGCTT |
| | | ATTGTTGAGGAGGAAGTGCCTGTGTCTGAGGTCGTCCATG |
| | | AAGTCCAAGATGGCTCTGAAGTGGTAGCGGAATCTGACAA |
| | | CAAAACTGGGGAAGTTCCTAAGAAGTCATATGCTTCAATT |
| | | GTCATGCATCTCAAGGAAAGTGCTGCAACCTTCTCCCCTC |
| | | CTCCAGCACCTGCTGCTAGGAAGCCTATGACAAAGAGTGT |
| | | TGTGCAAGTGAATCAGCCTGCTGCCACAACAACTGATGGC |
| | | CCATATTCCAGCTCAGAGACCATTGATAACATAAATGACA |
| | | CAGAAGCTGATGGCTACTCAATCTATATAAAAGGCCTGCC |
| | | TATGAGTGCAACTGTGGCATTACTTGCGGATGAGTTCAAG |
| | | AAGTTTGGGCCAATAAAAAATGGAGGAATTCAAGTGAGAA |
| | | GTAACAGGCAACAGGGATTTTGTTTTGGATTCGTGGAATT |
| | | TGAAGTGGAAAGTGCTGTGCAAAAAGCAATCGAGGCTTCT |
| | | CCAGTTCTCATTGGTGATCGGCAAGCAGTGGTCGAGGAAA |
| | | AGAGGTCTACCAATTCTCGACTTAATGCCAGAGGTGGTGG |
| | | TAGGTTTCAATCTGGAAGGGGCGGTGGATTCAGGAACGAT |
| | | GTTGGAGGAAGAGGGCGAGGAAACTATTTAGGTGGCAGGG |
| | | GTTATGGTCGAGATGATTTCCATGGAAGAAATGAGTTCAA |
| | | TAACAGGGGTGGGAATCGTGGGGGTTCATCAAACCGTGGA |
| | | GGTGAAGGATATCGGCGAGCTGACAATACAGGTGGGCGAA |
| | | TGAGTCGTGGTGGTGGAATGTCTAATGGAACAGCCAAAGG |
| | | CCCGGCTCCTCGCTATTCTGCTACAGCTTGA |
| SEQ | Solyc07g007930.3 | ATGCAATTGAGCTTCAGCAGACCAAAATCACCAATGATGT |
| ID | | TTGATTTGATTTCTATTTCTAGCCATCACCCTAATTCCAT |
| NO: | | CATCAATACTATAAATAGGCCAATCCAAAACCCCAAAATC |
| 199 | | CCATCATTCTTCTCTTCTACAACAACCCATTTTTCATACT |
| | | CATTCAACAAATATAGTAACCCTTTACAATTCTCTGTTTC |
| | | ATCACCTACCACTTCAGTTCTTGCTCACAAAGGAAGTGAA |
| | | GTAGAATTTGAGAAAGGGCTAGAAGAGGAGGAAGCAGCAG |
| | | CAAAAACAGTAGTTGACGCTGCCATGACGATTACTCCAGC |
| | | GATAAGGATTTCTGACAGAAAACTGATGGTGAAAGACAGA |
| | | ACAATATTAACAAATGTACCCGACAACGTACTCACCACTC |
| | | CCGGTGCTGCATCAGGTCCATTAGAGGGTGTATTTCTTGG |
| | | TGCAGAATTTGATCAAGACAACAACCGCCATGTTGTGCCT |
| | | CTCGGGAAATTACAGGATGTTCGGTTCTTGTCTTGTTTCA |
| | | GGTTCAAGTTATGGTGGATGGCTCAGAAAATGGGTGATAA |
| | | GGGAAGTGAAATCCCAATGGAAACACAATTTCTTCTTGTT |
| | | GAAACAACAGATGGATCTCATCTCGGATCCAACGACAACA |
| | | AAAATGATGATAATATTGTGTATGCTGTTTTCCTTCCATT |
| | | AATTGAAGGTTCATTTCGAGCTGTTCTTCAGGGTAATGCT |
| | | GAAGACGAGCTCGAATTATGCCTTGAAAGTGGCGATAAGG |
| | | ACACTGTTGGATCAGCATTTAATCAAGCAGTTTATATTCA |
| | | TGCTGGCAGTGATCCGTTTATCGTAATCACGGAGGCGATA |
| | | AGGGCGGTGAAGTTACATCTCAAGACTTTCCGCCAACGGC |
| | | ACGAGAAGAAGCTCCCCAAAATTGTTGATTACTTTGGGTG |
| | | GTGTACTTGGGATGCTTTTTATCAAGAAGTTACTCAAGAA |
| | | GGCGTTGAAGCCGGACTCAAAAGTCTTACAGCTGGTGGTA |
| | | TTCCGCCAAAATTCATCATCATCGACGATGGCTGGCAGTC |
| | | AGTCGGTGGTGATCCAGAAGTTGATAAACCATTGATGAGA |
| | | CTTACAGGATTAAAAGAAAACGAAAAATTTCAGAAGAAAG |
| | | AAGATCCAACATTGGGGATTAAAAACATTGTGAATATAGC |
| | | TAAAGAGAAATACGGCCTGAACTATGTGTATGTATGGCAC |
| | | GCAATAACGGGTTATTGGGGCGGAGTCCGACCCGGAGTTA |
| | | AGGGGATGGAGGAATACGGATCGGTTGTGAAGTACCCAGA |
| | | TATTACAAAAGGAGTGATGGAGAATGAACCGGGATGGAAA |
| | | ACGGATGCAATTGCGGTTCAAGGTTTGGGTTTGGTTAACC |
| | | CGAAAAGTGCTTATAAGTTTTATAACGAAATGCATAGTTA |
| | | TTTGGCATCAGCTGGAGTGGATGGGTTGAAGGTGGATGTG |
| | | CAGTGTATACTGGAGACTTTAGGGGGTGGTTTAGGGGGTC |
| | | GGGTTGAGCTCACTAAACAGTATCATCAAGCTCTTGATGC |
| | | TTCTGTTGCTCGGAATTTTCCTGATAATGGTTGCATTGCT |
| | | TGCATGAGCCACAATACTGATGCACTTTACTGTTCAAAGC |
| | | AGACGGCAGTTGTGAGAGCATCAGATGACTTCTATCCAAG |
| | | AGATCCAGCTTCGCACACCATTCACATTGCTTGTGTGGCA |
| | | TACAACAGTGTGTTCCTTGGAGAAATTATGCTGCCTGATT |
| | | GGGACATGTTCCACTCCCTTCATCCAGCAGCCGAGTATCA |
| | | TGGCTCAGCGAGGGCGTTGAGTGGTGGACCTGTCTATGTG |
| | | AGTGATGCACCTGGCAAGCACAACTTTGATGTTCTGAGGA |
| | | AGCTTGTTCTTCCCGATGGTTCAATTCTTCGTGCTCGTTT |
| | | GCCTGGTCGACCTACAAAGGACTCCCTTTTCACTGATCCT |
| | | TCTCGTGATGGTGTTAGCCTTTTGAAGATATGGAACATGA |
| | | ACAAATACACTGGTGTTCTTGGAATATACAACTGCCAAGG |
| | | TGCTGCGTGGAGCACGGTTGAGAGGAAGACCACATTCCAC |
| | | AAAACCAACTCAGAGGCGATAACAGGCTATATCAGGGGCT |
| | | GTGATGTCCATTTCATATCTGAAGCTGCCTTGGACCCCAA |
| | | CTGGAGTGGAGACACAGTTCTTTACTCTCATGGAAGTGCT |
| | | GAGCTTGTTGTTCTCCCGTATAATGCAGCAATGCCTGTTT |
| | | CTTTTAAGATCCTTGAGCACGAGACATACACTGTCACGCC |
| | | TATTAAGGTCTTGGCTCCTGGCTTCAGCTTTGCACCTTTG |
| | | GGGCTTATTGATATGTACAATGCTGGTGGGGCAATTGAAG |
| | | GACTAAAATACGAGGTGAAGGCAGGCGCAGAATTGTCTGA |
| | | ACTCGAGGCTGGATATCAAGGTGAAGGAAATCTTGTGGCT |
| | | GAAGATAAAATTGAGAACTTGAGCACAGAAGCAGTTGCAG |
| | | TAGTGTCGATGGAAGTAAGGGGATGTGGCCGATTTGGTGT |
| | | TTACTCATCTGTCAAGCCGAGGAAGTGCAGTGTGGGTGGA |
| | | GATATGGTTGATTTTGCCTATAACTCAGAATCTGGTTTGT |
| | | TAACTTTGAATCTTGATGCTATGCCTCCAGCAGATCAGAA |
| | | AGTGCACATCATTGAAGTTGAAGTATAG |
| SEQ | Solyc07g015980.4 | ATGGCTTCAACTTGTCTGGATGTGAGCTTTCTTGGGAATT |
| ID | | TTTGCAACAACTGGAGTTTCAGACCTTGTACAAGACTAAC |
| NO: | | ACCTTGCATTTCCCAAGTAAGGAATAGTTTATTGCTTGGG |
| 200 | | AAAAACAAGAACTATACTAGCTCTGTAGGATATAAGGGAA |
| | | TTCCGTTAACAGAAATTTCTTGTGTTCCAAGTGCTGTTGC |
| | | GAGTTCTAGTGAACTTCCTCCACTTGTTAGTGCTTTAAAA |
| | | GCTTCAGCTGAAGAAAATGCTGCAAGTTTTCATTTTCCTG |
| | | GCCATAATAGAGGACAAGCAGCGCCATCATCACTAACTCA |
| | | GCTCATTGGTGCAAAACCGTTTCTTCATGATATGCCTGAG |
| | | CTTCCGGAGCTTGACAATCTGTTTTCACCAGAAGGTCCCA |
| | | TTCTCGAAGCACAAAAAGAAGCAGCAAGACTTTTTGGAGC |
| | | ATTGGAAACATGGTTCCTTGTTGGCGGTAGTACATGTGGA |
| | | GTTCACGCGGCCATTATGGCGACTTGTTCACCTGGAGATA |
| | | CTCTTATTCTCCCACGGAATTCTCATATCTCAGCCATATC |
| | | TGCCATGGTATTGTCTGGTGCACTTCCTAAGTATATCGTC |
| | | CCTGAATATGATTTCAAATGGGATGTTGCTGGTGGCATCA |
| | | CTCCATCACAGGTGAATGATGCAATCAAAGAGCTAGAAAT |
| | | GGAAGGCCGAAAAGCAGCAGCCCTTTTGGTCGTCTCCCCT |
| | | ACCTATCATGAGTTGCCTAGCTCATCCCTCAGCCAGGGAG |
| | | CTGATCTTGCTGTACAGTCTACTCACAAAGTTTTGTGTTC |
| | | GCTTACGCAGTCATCTATGTTGCACATGCAAGGAAATCTT |
| | | GTTAATAGAGAAAGAATCAGCAAAAGCCTTCAAATGCTTC |
| | | AAAGTAGTAGCCCTAGTTATCTGCTTTTAGCATCTCTGGA |
| | | TGCTGCCAGGGCCCAATTAAGTGAAAATAGAGAGGCTGTT |
| | | TTTGACAAAGCAATGGACTTGGCTCTGGAAGCAAGAAGCT |
| | | TGATTAGTAAAATTCCTGGAATCTCAATCATTGAGTTCCC |
| | | AAGTTTCTCAAGTTTTTTCCAGATCGACCCATTGCGACTC |
| | | ACCATTGGTGTATGGCAGTTAGGTCTTTCAGGTTTTGAAG |
| | | CAGACGATATCTTGTGCAATGATTTTGGTGTTGTTTGTGA |
| | | ACTTGTTGGGACCAAATCTTTTACTTTGGCATTTAACTTA |
| | | GGGACTCAAAGAGATCATGTCCTTCGGCTTGTAGCTGGTC |
| | | TAAACCATCTATCACAAACTTCTCACTTTCCTCAACCTGT |
| | | GAAAGAAGAAGGCAAAAATGTAAATCATTTTGTATGCCTT |
| | | GATGATATTAGAATAAGAATGAGTCCTAGAGAGGCCTTCT |
| | | TTGCTACAAAACGTAAAGTCAGCATAAGAGATAGCCTCGG |
| | | TGAAATATGTGGAGAGCTTGTATGTCCTTATCCACCTGGT |
| | | ATTCCAGTTCTAATTCCTGGTGAGATCATCACAGCGGAAG |
| | | CATTGAATTATCTCCTGGAGATCAGAAGCAAGGGTGCTGT |
| | | TATTACCGGAGCGGCTGATTTCTCCCTATCATCTTTTGTT |
| | | GTATGTGTTACCTAA |
| SEQ | Solyc07g062700.3 | ATGAGAAGATCTCCAAATATGTTCCCCAAATCGTTCTTCA |
| ID | | CTCTCCTATTTATCTTCATTTTATCTACCGCCGTGTACGG |
| NO: | | CGGCCGGGTAATCGTCGATCAGTCATCGTCGGATCTCATC |
| 201 | | TCCGATGGAGTTCACGGCGGCGGAGGCAGAAATAGTACTA |
| | | GTATTGGCCGTGAAAGAGGAATTATCTCGCTATTTGCTGC |
| | | ACCAGTAGAAGAGGCATGTGAACAGAGCTATGGATTTTTG |
| | | CCGTGCACTAAAACTGCTCTAGGAAATGTGTTCCTTATTG |
| | | TTGTGTATGGTTACCTTATGTTTACGGCGGCTACGTACCT |
| | | CTCCTCCGGTAGCGAGTTATTGCTTGAAATTTTGGGGCCG |
| | | GGTCTAATCGGTGGACTTTTCCTGCCGATTCTCGGTGCTC |
| | | TTCCTGATGCTATGCTTATTCTCGTATCCGGGATTACTGG |
| | | AAGTGCTGCAGAAGCTCAGAGCCAGGTCTCTGTAGGAATA |
| | | GGCTTGTTAGCTGGGTCGACAGTGATGCTTCTTACTGCGA |
| | | TATGGGGAACCTGTTGCACTGTTGGCAAGTGTGATATAGT |
| | | AAATTCTATTGCTGTGGACTTGAAAGACACTAAAGGGCTC |
| | | AGTTTTACTGGTTCTGGTGTTAGTACTGATATCTGGACAA |
| | | GCTATGCTGCAATGATCATGGCCGTATCTGTTCTTCCATT |
| | | TATTGTTGTGCAGTTACCACAGATCTTCCATTCAAATTCA |
| | | GGAAGACATTTATCTGTCTTGATAGCTCTTATCATATCAT |
| | | TGTCGCTGCTCCTCTCTTATTGTCTGTATCAGGTCTTTCA |
| | | GCCTTGGATACAAACACGAAAACTTGATTATGTAAAACAT |
| | | AAGCATGTTATATCAGGAATCTTGAAGCATTTAAAAAAGC |
| | | ACGCATTAGGGAGGCTTTTCACAGATCATGGAACACCAAA |
| | | TATAGAAGTTTTAGAAAAGCTGTTCAATGCAATTGATGAA |
| | | AATGGTGATAAACATCTTTCCCATTCTGAATTAAGAGCTC |
| | | TGGTCGTAGGAATCCGTCTTGAGGAGATTAACTTAGATGA |
| | | AAATGATGCAATAGCAAAGGTTATGAAGGACTTCGATACG |
| | | TCTCTTGATGAACAAGTTGACCTGCATGAATTCATTACTG |
| | | GAGTTGAAAAATGGCTTTATGAAGCCAAGAGTACTAATGG |
| | | TTCTTCTGCTCAGGCTGGTCCTAATACCATCAACTACATT |
| | | GATGATTTTCATGAGGAAACAAGGAGAGAGCACAGTCTCT |
| | | TGGGGGAGGATCAAAGTGATGAAATTGTTGAGGGCGTCGA |
| | | GAATCCTCGAAAAGTTGCTATTAAGGCCGCGTCATTGTTG |
| | | TTGTTGGGTACCATTATTGCTGCTGTCTTTGCTGATCCTT |
| | | TGGTGGATGCTGTTAACAATTTCTCCAGTGCCACAAGTAT |
| | | TCCATCTTTCTTTATCTCATTCATTGCACTGCCTCTGGCA |
| | | ACAAACTCCAGTGAGGCAGTGTCGGCTATAATTTTTGCTA |
| | | CCCGGAAAAAGCAGAGATCCGCGTCATTAACATTTTCTGA |
| | | GTTATATGGAGCTGTGACTATGAACAATCTCCTTTGTCTC |
| | | TCAGTCTTCTTGGCTATTGTTTATGCCCGAGGTCTAACAT |
| | | GGAACTTCTCATCTGAAGTGCTGGTTATCCTAATTGTTTG |
| | | CATTGTTATTGGGGTCTTAGGGAGCATCCGCACCACCTTT |
| | | CCTCTGTGGACTAGTTTGATAGCTTTTGCTTTTTATCCGT |
| | | TCTCCCTTGTACTCGTGTATGTTCTTGACTACGTCTTTGG |
| | | TTGGTCATAG |
| SEQ | Solyc07g064130.2 | ATGCAGATCTTTGTGAAAACTCTCACCGGAAAGACTATCA |
| ID | | CCCTAGAGGTGGAAAGTTCTGATACAATCGACAACGTTAA |
| NO: | | GGCTAAGATCCAGGATAAGGAAGGAATTCCCCCGGATCAG |
| 202 | | CAAAGGCTTATCTTCGCTGGAAAGCAGTTGGAGGACGGAC |
| | | GTACTCTAGCTGATTACAACATCCAGAAGGAGTCCACCCT |
| | | CCATTTGGTGCTCCGTCTACGTGGTGGTATGCAGATCTTC |
| | | GTGAAGACTCTCACGGGTAAGACGATTACCCTTGAGGTCG |
| | | AAAGCTCAGACACCATTGACAACGTCAAGGCTAAGATCCA |
| | | GGATAAGGAAGGCATTCCCCCAGACCAGCAGAGGTTGATC |
| | | TTTGCAGGAAAGCAGTTGGAAGATGGCCGCACCCTAGCTG |
| | | ACTACAACATCCAGAAGGAGTCCACCCTCCATTTGGTGCT |
| | | CCGTCTCCGTGGTGGTATGCAGATCTTCGTTAAGACTCTT |
| | | ACCGGAAAGACCATCACTTTGGAGGTGGAAAGCTCCGACA |
| | | CCATTGACAACGTGAAGGCTAAGATCCAGGATAAGGAAGG |
| | | GATCCCCCCAGACCAGCAGAGGTTGATCTTCGCTGGAAAG |
| | | CAGCTCGAGGATGGTCGCACCCTGGCTGACTACAACATCC |
| | | AGAAGGAGTCTACCCTCCATCTTGTCCTCCGTCTCCGTGG |
| | | TGGTATGCAGATTTTTGTTAAGACCCTCACCGGAAAGACC |
| | | ATCACTTTGGAGGTGGAAAGCTCCGACACCATTGATAATG |
| | | TTAAGGCTAAGATCCAGGACAAGGAGGGAATTCCTTCAGA |
| | | CCAGCAGAGGTTGATTTTCGCTGGTAAGCAGCTCGAGGAC |
| | | GGCCGCACCCTTGCCGACTACAACATCCAGAAGGAGTCGA |
| | | CCCTTCACCTTGTCCTCCGTCTACGTGGTGGTATGCAAAT |
| | | CTTTGTGAAGACCCTTACCGGGAAAACCATCACCCTGGAG |
| | | GTTGAGAGCTCCGACACCATTGACAATGTCAAGGCCAAGA |
| | | TCCAAGACAAGGAGGGTATTCCCCCAGACCAGCAGAGGTT |
| | | GATTTTTGCTGGCAAGCAGCTCGAGGATGGCCGCACTTTG |
| | | GCGGACTATAACATCCAAAAGGAGTCGACCCTGCACTTGG |
| | | TGCTTAGGCTGAGGGGAGGAATGCAGATCTTTGTGAAGAC |
| | | CTTGACCGGGAAGACCATCACTTTGGAGGTGGAGAGTTCT |
| | | GACACCATCGACAATGTGAAAGCTAAGATTCAGGACAAGG |
| | | AGGGGATCCCACCAGACCAGCAGAGGTTGATTTTCGCTGG |
| | | TAAGCAGCTTGAGGATGGCCGCACCCTTGCTGACTACAAT |
| | | ATCCAGAAGGAGTCCACCCTGCACCTTGTCCTCCGTCTCC |
| | | GTGGTGGTTTTTAA |
| SEQ | Solyc07g065430.2 | ATGGGCTGGCTGCGGTATGTGGAATCTGGGAGGAAAATTG |
| ID | | ACTGCATTAGCAATCCTAACACCGCATACCCCATTCCAGT |
| NO: | | CCTTGTTGAAGAAGTTGATGATATTATTAGCGTTGCTCAA |
| 203 | | TACGTTATAGTTGTGGAGAAGGAATCAGTATTCCAGCGAC |
| | | TAGGAAGGGGTTATCCTGATGTTCCCACAAGGAGGTTTCT |
| | | GCGTCTCCTCATTGACAAGCTGCATTTGCCTGTGTACGGC |
| | | CTGGTAGATTGTGATCCATATGGCTTTGACATCTTAACTA |
| | | CATACAAATTCGGTTCTTTGCAAATGGCTTATGACGCAAC |
| | | ATTTCTACGAGTCTCTGAGATACAATGGCTTGGAGTATTT |
| | | GTCCAGGATTGTGACAATTATAGCATTCCACAGCAATGTC |
| | | TCCTGCCTTTAACTGCTGAAGATAAGAGGAAAATCGAAGC |
| | | AATGTTACCTAGATGTTATTTGCAAAGAGAAGTACCACAA |
| | | TGGAGGTTTGAACTGGAGCTGCTGTTGCACAAAGGAGTTA |
| | | AGTTTGAGATTGAAGCATTGTCTGTGCATTCACTCACTTT |
| | | CTTGTCCCATGAGTATCTACCATCAAAGATCCACAATGGA |
| | | GGGTATATAGTGGACGTTCATTGTGATGCATGCAAGATGA |
| | | AGATGGTAGATGTTTTGAGTTCTATATGCGGTGTTTATTC |
| | | TGTCACAATAGACTCTGAAGATGGGACAGCCAAGATTTCT |
| | | GGTGAAGTTGATCCGAACCTTCTCTTGAGGGCGTTGTCAA |
| | | GATCAGGCCTTGGCAACCATGCAGAGGTGAAGTGGGTGAG |
| | | ACTTAAGCACCCAATGTTGAGTAATAGTCATATGACTGAT |
| | | GGATACGGTTCCTACAATCATGGATATGGCTCTTATAATC |
| | | ATGGATATGGCTCTCATAATCACGGATATGGTTCGTATAA |
| | | TCCTGGCTACGGTTATAACAGTTATGGCCACGGTCATGGC |
| | | CATGGCCATGGCTATAGTTCTATTGGTGGTCCTTTTATGC |
| | | AAAGAAGATCGTTGCCTGAATATAATTATGGCTATGAGGG |
| | | ACACCACAATTACCAATATCCTTTTAGGGGGACTGCACAA |
| | | GACTCCTATGCCACCAACTATTACTCCGGTGCTCTGCCAT |
| | | CACCTAGGCCGTACAACAGCTATTATATGTAG |
| SEQ | Solyc07g065840.2 | ATGTCGGACGTAGAGACGTTTGCTTTCCAGGCTGAGATCA |
| ID | | ATCAGCTTCTCAGTCTTATCATCAACACTTTCTACAGCAA |
| NO: | | CAAGGAGATCTTTCTCCGTGAACTCATCAGCAATTCTTCC |
| 204 | | GATGCTCTAGACAAGATCCGCTTTGAGAGTTTAACCGACA |
| | | AGAGCAAGCTAGATGGTCAACCAGAGCTCTTCATCCATAT |
| | | TATTCCAGACAAGGCCAATAATACTCTCACTATCATTGAT |
| | | AGTGGTATTGGTATGACAAAGGCTGATCTGGTGAATAACC |
| | | TGGGTACAATTGCAAGGTCAGGAACCAAGGAGTTCATGGA |
| | | AGCTCTTGCAGCTGGTGCTGATGTTAGCATGATTGGTCAA |
| | | TTCGGTGTAGGTTTCTACTCTGCTTACTTGGTAGCTGAGA |
| | | AGGTTGTTGTGACCACAAAGCACAATGATGATGAGCAATA |
| | | TGTCTGGGAGTCTCAAGCCGGTGGCTCTTTCACTGTTACC |
| | | AGGGATACATCTGGTGAGAACCTTGGTAGGGGTACCAAGA |
| | | TGGTCCTTTATCTCAAGGAGGATCAGCTTGAATACCTTGA |
| | | AGAACGTAGGCTCAAGGACCTGATTAAGAAGCACTCTGAG |
| | | TTCATTAGCTATCCTATTTCTCTGTGGGTTGAGAAGACCA |
| | | TAGAGAAGGAAATTTCTGATGATGAGGAGGAAGAGGAGAA |
| | | GAAAGATGAGGAGGGAAAGGTAGAGGAGGTCGATGAGGAA |
| | | AAGGAGAAGGAAGAGAAGAAAAAGAAGAAGGTCAAAGAAG |
| | | TTTCCAATGAGTGGTCACTGGTGAACAAGCAGAAGCCTAT |
| | | TTGGATGAGAAAGCCAGAAGAGATCACAAAGGAAGAGTAT |
| | | GCTGCTTTCTACAAGAGCTTGACTAATGATTGGGAAGAGC |
| | | ATCTTGCTGTGAAGCACTTCTCTGTTGAGGGTCAGTTGGA |
| | | GTTCAAGGCTGTCCTTTTTGTTCCAAAAAGGGCTCCTTTT |
| | | GACCTCTTTGACACAAAGAAGAAGCCCAACAACATCAAGT |
| | | IGTATGTTCGCCGTGTGTTTATCATGGATAACTGTGATGA |
| | | GTTGATTCCTGAATATTTGAGCTTTGTGAAGGGTATTGTG |
| | | GATTCTGAGGACCTTCCTCTCAACATCTCCAGAGAGACAT |
| | | TGCAGCAGAACAAGATCCTAAAGGTTATTCGCAAGAATTT |
| | | GGTGAAGAAGTGTGTTGAGCTTTTCTTTGAAATTGCTGAG |
| | | AACAAGGAGGACTACAATAAGTTCTATGAGGCGTTCTCTA |
| | | AAAACCTCAAGCTTGGAATCCATGAGGATTCTCAGAACAG |
| | | GGCAAAGTTTGCTGAACTGCTGAGGTACCACTCCACTAAG |
| | | AGTGGTGATGAGATGACCAGCTTGAAGGACTATGTGACCA |
| | | GAATGAAGGAGGGCCAGAATGATATTTACTACATTACTGG |
| | | TGAGAGCAAGAAGGCTGTTGAGAACTCTCCCTTCCTGGAG |
| | | AAACTGAAGAAGAAGGGATATGAGGTGCTTTACATGGTTG |
| | | ATGCCATTGATGAGTATTCAATTGGTCAGCTGAAGGAATT |
| | | TGAGGGCAAAAAGCTTGTTTCTGCTACCAAGGAAGGCCTC |
| | | AAGCTTGATGAGAGTGAAGATGAGAAGAAAAAGCAGGAAG |
| | | AATTGAAGGAGAAGTTTGAGGGACTGTGTAAGGTGATGAA |
| | | GGATGTGCTAGGAGACAAAGTTGAAAAGGTCATTGTTTCT |
| | | GACCGTGTTGTTGACTCTCCCTGCTGTTTGGTCACTGGTG |
| | | AGTATGGCTGGACTGCTAACATGGAGAGAATTATGAAGGC |
| | | ACAGGCACTTAGGGACTCCAGCATGGCTGGATACATGTCT |
| | | AGCAAGAAGACCATGGAGATCAACCCAGAGAACTCCATCA |
| | | TGGATGAGCTAAGGAAGAGGGCTGATGCAGACAAGAATGA |
| | | CAAGTCTGTGAAGGACTTGGTTCTCTTGCTTTTTGAGACT |
| | | GCCCTTCTCACCTCAGGTTTCAGCCTCGAGGAGCCAAACA |
| | | CCTTTGGCAACAGAATTCACAGGATGTTGAAACTCGGTTT |
| | | GAGCATTGATGAGGAAAGCGGAGATGCTGATGCTGACATG |
| | | CCAGCATTGGAGGATCCTGAAGCTGATGCTGAGGGCAGCA |
| | | AGATGGAGGAGGTTGATTAA |
| SEQ | Solyc08g006970.4 | CCATTTGAACTCGGAGATAATAGTTACAAGCATAATAAAT |
| ID | | ACATAATCCAAACAATTAATTTCTCCATCATTTCTTCTTT |
| NO: | | GTTCACCATGGCTACTCCAATCACAAAACCCCCATCACCT |
| 2054 | | GCTTGTACCAAAATCATAGAAGAAATCACAAGGATTTACA |
| | | AATCACTACCACCAAGACCATCCATTTTAGAGATTGAAGC |
| | | TTCAATTTCTGTTGTAAAATCAGTAGAAACAGAAGAAAAA |
| | | ATAGAACTTGATGAAATTTCAAAGAGAAGAGTTGAGATAG |
| | | AAGAAAGTGTTCCAGCTGAGCTTTACTCTGTGTTAGAAAA |
| | | AGTGAGAAAAGCTATGGTTCTGTTCCAAAGCAAAGAACAG |
| | | AAGAAAGAGGCTGTTGAGTTGATTGAGCTTGATAAGACTT |
| | | ATCAAGATTTTGATGAGTTGATTCAGGAAGCTACAGAGTT |
| | | GGTTTGTGAAGATACCCAAATGGGAAAAATCAATTCTTTT |
| | | GAGGACCCAATGGTTGAAATTGGAAAAAAGGATGAGGTTT |
| | | TGAAGAAGGGTTTGGTTACAAGTGGTGAATTGATTAATGT |
| | | TTCTTCTTCAGGGATCAAGCACAAAGAGAAGTACAGCTTG |
| | | ATGAAAGTAGCTGCCCTAATTGAAAATGCCGCCAAAACTC |
| | | GAGCAAGAGTTGTTGATCTTCAGAACAAGTTAATGGACAA |
| | | GATTGAATGGCTTCCATTGTCACTTGGGAAATTGGTGAAT |
| | | GTCACTGAGCTAAACGTTGCCGATAACCAGATCATGGCTC |
| | | TTCCAACTACCATTGGTAGCCTTAATGGCTTAACGAAGCT |
| | | CGATCTTCACTCCAACCAAATTATAAACCTTCCTGATTCA |
| | | TTTGGTGAGCTAATCAATTTGACTGATCTTGACCTCCACG |
| | | CGAACAGGTTGAAATCGCTGCCAGCTTCTTTCAGGAACTT |
| | | AGTAAATCTGATCGATCTTGATTTGGGTTCGAACAGGTTC |
| | | ACTCATTTGCCTGATTTCGTTGGGAACTTAACCTCCTTGA |
| | | AGAGACTAAACGTGGAAACAAATCAGCTCGAGGAACTTCC |
| | | TTACACTATAGGATTTTGTTCTTCCCTTGTAGAACTGAGA |
| | | TTGGATTTTAATCAGCTTAAAGCTCTCCCCGAGGCAATGG |
| | | GAATGCTCGAACACTTGGAGATTCTTACGCTACACATTAA |
| | | CAGAATCAAAGGATTACCAACAACAATGGGGAATCTTTCT |
| | | CGCCTAAGGGAACTTGATGTTAGCTTCAACGAAGTTGAGA |
| | | ACATACCCGAAACATTCTGTTTTGCTGTCAGCTTGGAAAA |
| | | GCTTAATCTTGCAAACAACTTTGCGGACTTGAAAACTTTA |
| | | CCAAGATCAATCGGAAACCTTGAGAATCTTGAAGAGCTTG |
| | | ATATTAGCAACAGTCAAATAAGAACGTTGCCCGACTCCTT |
| | | CAGGCTCTTGTCAAAGTTGAAAACTTTTCGAGCTGATGAG |
| | | ACTCCGCTAGAAGTACCACCAAGGCAAATTATAAAGCTCG |
| | | GGGCTCAGGTTGTTGTAGAGTACATGGCTGAATTCGTAAC |
| | | TAAGAACGAGCTTCAATTGCAGCGGCCTAAGAGGAGACCT |
| | | TTCTTTTGTCCAAGTTGCCTGTTTCCTAAAACCGAGGGGA |
| | | AGAGGCGGATCTAG |
| SEQ | Solyc08g029110.3 | ATGGAGAAAATCAGGAAAGCATCTCATGCTGGTTCTTGGT |
| ID | | ACACTGATAATCCCCTAGAACTAGAAGAACAGCTTGATGG |
| NO: | | CTGGCTGAGAGCTGCTGGCTTAGTCAAGTCTTCTGATGTA |
| 206 | | CGAGGTGTAATTGCACCTCATGCAGGCTATTCATATTCTG |
| | | GTCGTGCAGCAGCTTATGCATTTGGGAACATAGACCCGAC |
| | | CAATATTTCTCGGATATTTCTACTCGGTCCATCTCACCAC |
| | | TATTATACTCCAAAATGTGCACTTTCAAGAGCCACAGTTT |
| | | ACAAGACTCCTATAGGAGATTTACCCATTGATGAAGAAGT |
| | | AAATGACGAGCTAAAAGCTACGGGGCACTTTGAATACATG |
| | | GATCTTAGAGTTGATGAAGCTGAACATAGCATGGAAATGC |
| | | ACCTTCCATATCTTGCTAAAGTTTTCCAAGGGTATCCAGT |
| | | GAAAATTGTGCCCATTCTGGTTGGGGCTCTTAGTGCTGAA |
| | | AGTGAAGCCCTTTATGGACGGTTTCTCGCAAAATATGTGG |
| | | ATGACTCGAAGAATTTCTTCTCAGTGTCTTCGGATTTTTG |
| | | TCATTGGGGATCAAGGTTCAACTACATACATTATGACAAA |
| | | AGCCATGGAGCTATTTACAAATCTATTGAAGTCTTGGATA |
| | | AAATGGGTATGGATATTATAGAAACAGGAGATCCAGATGC |
| | | ATTCAAACTGTACCTGTCAGAGACGGACAACACCATTTGT |
| | | GGACGCCATCCAATCAGTGTTTTTCTTCATATGTTGAAAA |
| | | ACTCGTCGACTAAGATAAAGATTAGATTTCTTCGATATGA |
| | | ACAATCGAGCCAGTGCAAATCTATGAGGGACAGCAGCGTA |
| | | AGCTATGCATCGGCAGTGGGAAAAGTTGATGGTTGA |
| SEQ | Solyc08g065220.3 | ATGGAGCGTGCTAGAAAATTGGCAAACAGAGCAATTCTGA |
| ID | | AACGTTTGGTTTCACAATCAAAACAGAGTCGTTCTAATGA |
| NO: | | AATCCCATCACCATCATCATTATACAGACCTTCAAGGTAT |
| 207 | | GTATCTTCACTGTCACCATACACATTTCAGGCTAGAAATA |
| | | GTGTGAAATCTTTTAATACTCAACAAGTTAGATCAATTTC |
| | | TGTTGAGGCTTTGAAACCAAGTGACACATTCCCACGCCGA |
| | | CATAACTCAGCCACCCCTGAAGAACAAACTAAAATGGCTG |
| | | AGTTTTGTGGGTTTCAAAGCCTAGATGCACTTATTGATGC |
| | | TACTGTACCTCAATCGATTCGTAGTGAATCGATGAAGTTA |
| | | CCTAAGTTTGATGGTGGATTGACTGAGTCACAGATGATTG |
| | | AGCATATGCAGAAATTGGCATCTAAGAATAAGGTTTTTAA |
| | | GTCATATATTGGGATGGGGTATTATAATACTTATGTACCA |
| | | CCTGTTATATTGAGGAATCTTTTGGAGAATCCTGCTTGGT |
| | | ATACTCAGTATACTCCTTATCAGGCTGAAATTTCCCAGGG |
| | | ACGTCTTGAGTCCCTGCTGAATTATCAGACCATGATTACG |
| | | GATCTTACTGGTTTGCCAATGTCTAATGCATCTTTACTAG |
| | | ATGAAGGGACCGCCGCAGCTGAGGCTATGGCTATGTGTAA |
| | | CAATATTCTTAAGGGAAAAAAGAAAACTTTCCTTATTGCT |
| | | AACAATTGTCACCCTCAGACTATCGAGATTTGTAAGACTA |
| | | GAGCTGATGGATTTGATCTTAAGGTAGTTACTGTAGATCT |
| | | TAAGGATATTGATTATAAGTCTGGTGATGTTTGTGGGGTA |
| | | CTAGTTCAGTATCCGGGGACCGAAGGTGAAATCTTGGATT |
| | | ATGGGGAGTTTATTAAGAATGCACATGCTCATGGGGTGAA |
| | | GGTTGTTATGGCATCTGATCTTTTGGCCTTGACAATGTTG |
| | | AAACCACCTGGTGAGCTTGGAGCGGATATTGTTGTTGGTT |
| | | CTGCTCAGAGGTTTGGAGTGCCTATGGGTTATGGAGGCCC |
| | | TCATGCAGCTTTCTTGGCAACTTCTCAAGAATACAAGAGA |
| | | ATGATGCCTGGAAGAATTATTGGTCTCAGTGTTGATTCAA |
| | | CAGGGAAACCTGCTCTGCGTATGGCAATGCAGACTAGGGA |
| | | ACAACACATCCGCAGGGACAAGGCTACGAGTAACATTTGC |
| | | ACAGCACAGGCTTTGCTTGCCAACATGGCTGCCATGTATG |
| | | CTGTCTATCATGGACCCGAGGGGCTAAAAACCATTGGCCA |
| | | ACGTGTTCATGGTCTTGCTGGAACATTTTCTGCTGGTCTC |
| | | AAAAAGCTAGGAACAGTAGAAGTTCAGGATCTTCCATTCT |
| | | TTGACACTGTGAAGGTGAAGTGTTCTGATGCAAAGGCAAT |
| | | TGCTGATGTTGCTACCAAGAATGACATTAACGTGCGGATT |
| | | GTGGACAACAATACTATAACTGTATCTTTTGATGAAACTA |
| | | CTACCCTAGAAGATGTGGACGATCTATTCAAAGTTTTTGC |
| | | CTTGGGAAAGCCAGTCCCGTTCACTGCTCAATCAATTGCA |
| | | CAAGAGGTTGAGAACCTGATTCCTTCTGGACTTACAAGGG |
| | | AGACTCCATTTTTGACTCACCAAATATTCAACTCGTACCA |
| | | TACTGAGCACGAGTTGCTGAGATACCTCCATAAGCTGCAA |
| | | TCAAAGGATCTCTCCTTGTGCCATAGCATGATTCCTTTGG |
| | | GCTCCTGCACAATGAAATTGAATGCCACAACAGAGATGAT |
| | | GCCGGTGACATGGCCTAGCTTTGCAAATATTCACCCTTTT |
| | | GCACCCACTGAACAGGCAGCTGGCTATCAGGAAATGTTCG |
| | | ACGATTTGGGCGCCCTACTGTGTACAATAACAGGTTTTGA |
| | | TTCCTTCTCGTTGCAGCCTAACGCTGGTGCTGCTGGAGAA |
| | | TATGCTGGATTGATGGTTATTCGTGCATATCATATGTCAA |
| | | GGGGTGACCATCACCGCAATGTATGCATCATTCCTGTATC |
| | | AGCTCATGGAACAAATCCTGCAAGTGCTGCAATGTGTGGG |
| | | ATGAAAATTGTTGCTGTCGGGACAGATGCAAAAGGAAACA |
| | | TTAATATTGAAGAGTTGAGGAAGGCTGCTGAGGCACATAA |
| | | GGATAACCTTTCTGCTCTCATGGTTACATACCCATCAACA |
| | | CATGGAGTGTATGAGGAAGGAATTGATGAGATATGTAAGA |
| | | TTATCCATGACAATGGTGGTCAGGTGTACATGGATGGAGC |
| | | TAACATGAATGCACAGGTTGGTTTGACAAGCCCTGGCTTT |
| | | ATTGGTGCTGATGTTTGTCATCTAAATCTTCACAAAACAT |
| | | TCTGCATTCCTCATGGTGGAGGAGGTCCTGGAATGGGTCC |
| | | AATTGGAGTGAAGAAACACTTGGCACCATATTTACCATCA |
| | | CACCCTGTGGTATCAACTGGAGGGATCCCATCCCCCGATC |
| | | AGAGTAAGCCACTTGGTGCTATTTCTGCTGCACCCTGGGG |
| | | TTCAGCACTTATTTTGCCGATTTCATATACCTACATTGCC |
| | | ATGATGGGGTCTAAGGGACTTACAGATGCATCAAAGATAG |
| | | CTATCCTGAGCGCAAACTACATGGCGAAACGTTTGGAGAA |
| | | GCACTACCCAGTCCTCTTCCGAGGTGTCAATGGAACATGT |
| | | GCCCATGAGTTTATCATTGACCTGAGGGGCTTTAAGAACA |
| | | CTGCTGGGATAGAACCTGAAGATGTTGCTAAACGTCTTAT |
| | | TGACTACGGATTTCATGGACCTACAATGTCTTGGCCAGTT |
| | | CCTGGTACACTTATGATTGAACCTACTGAAAGTGAAAGCA |
| | | AGGCGGAACTAGACAGGTTTTGTGATGCACTCATCTCCAT |
| | | CAGAGAAGAAATCGCTCAGATTGAGAAAGGGAATGTCGAT |
| | | ATTAACAACAATGTTCTCAAGGGGGCTCCTCATCCACCAT |
| | | CAATGCTCATGGCAGATGCATGGACAAAGCCATATTCTCG |
| | | GGAATATGCTGCATACCCTGCTCCCTGGCTAAGGAGTGCC |
| | | AAATTCTGGCCAACTACAGGACGAGTGGACAATGTGTATG |
| | | GAGATCGCAACCTGATCTGCACCCTTCTTCCAGTGTCAGA |
| | | AATGGCGGAAGAAAAAGCTGCAACTGCTTAA |
| SEQ | Solyc08g069120.4 | ATGCATGTTGAACCCCACAGGCGAGCCCTGAGCGTTGAGC |
| ID | | GTTGGGTGTGCTTAGGGCGTGCAATCGGACGCTTAGGGCC |
| NO: | | CCACTTTTTGTTTCAACAGAACACCAAATCGAGCTCTTTC |
| 208 | | TTCTTCACTGCACTGTTGTTTTCCGCTAGGGTTTCAGGTA |
| | | TTAGTAGGTGTGTATTGAAAATGTCAAGAGTCTATGTTGG |
| | | AAATCTGGACCCTAGGGTCAGTGAAAGAGAGCTTGAAGAT |
| | | GAATTCCGCATCTTTGGAGTTATAAGAAGTGTTTGGGTTG |
| | | CAAGACGCCCCCTGGCTACGCTTTTATTGACTTCGATGAT |
| | | CGGCGGGATGCACAAGATGCAATCAAAGAGCTGGATGGTA |
| | | AGAATGGATGGAGAGTGGAGCTTTCACATAATTCTAGAGG |
| | | TGGAGGTGGTGGGGGCCGTGGAGGAGGTCGAGGTCGATCT |
| | | GGAGGCTCTGATTTGA |
| SEQ | Solyc08g075860.3 | ATGGACTATGGTTGCTTAAAAAAAATAGCCACAAAATCCC |
| ID | | CCACCACTACGATCTTTCTTCCCTATTTGCTCCGATCTGT |
| NO: | | GTTAACCACACGCTTTCAATTGCTTGTGGCCGGCTCATTC |
| 209 | | TGCAACTCCTCCGGCGACCATCGGAATTCGGAAGTTTTTA |
| | | CCTCTTTTTCCGGATCAAAAGAAATGGAGTTCTTCTTCAG |
| | | AACACAGACTGAGGAGACATCTGATTGCTCCTTCAATGGC |
| | | CAGGATATTCAAAGATGCCCGTTCTTGAGTAACATCAACA |
| | | AGCCAACAAACTTTTCTTTCTTTTCTGCTCTGAACTTTCC |
| | | CAGCCCTGTGAAGGGAGGTAAAGGTCCAATTTTTGAAGAT |
| | | GGCCCCAACTTCGATATGGCATTTAAAGTCTTCCATGGAA |
| | | AAGATGGGGTTGTCCCACTTTCTGGAAGGTCTCAATTTTT |
| | | CAATGACAACATTGAAGTAGAGTCTGCCCCTCAATTTAAT |
| | | CCTTTAGCTGCAAAAGCTGCCACCATAAGTTTGTCTTCAT |
| | | TCGGAGCAGGAGGTCCTTTTAGCTTTGATTCTTTCTCTCG |
| | | GAAATGGAATTCACAGAAGAAGAAGCCGGAGTCATCCAAA |
| | | AAGAAAAAGCCTTCTTCTCAGGACAAATCTTCTAAGCACG |
| | | AGGCAATGGGAAATGAGTGGTTGGAGTCAGGCAACTGTCC |
| | | TATCGCCAAATCTTATAGAGCTGTTAGCGGTGTTCTCCCA |
| | | CTTGTGGCCTCAGCTTTTCAGCTGCCTCCCGGTATGAAGC |
| | | TCAAATGCCCGCCTGCAGTTGTTGCAGCCAGGGCTGCCCT |
| | | CGCCAGGACTGCTTTTGTGAAGACTGTGCGGCCGCAACCA |
| | | CTATCTTCAAAGATGCTTGTTATTGGAGCTTTGGGCATGG |
| | | CAGCTAATATTCCACTAGGCATATGGAGAGAGCACACTGA |
| | | GAAGTTCTCACTTTCATGGTTTACTGCAGTCCATGCTGCT |
| | | GTTCCCTTCATAGCTATGCTGAGGAAGTCAGTTGTGATGC |
| | | CCAAAACAGCTATGGCATTAACCATTGCTGCTTCTATCTT |
| | | GGGACAGGTCATTGGCTCAAGAGCAGAGAGACTTCGAATG |
| | | AAAGCAAAAGCCGAGAGTGTTAAATTGGTAGCGCAGACTG |
| | | GCTCAGATGGGGTTATTGCACAGAGTAGCACAGAGGGTGT |
| | | TATTGCAGGTCTCAACTCAATCCAGGTCAGTGGTATGCCC |
| | | GGGGTTCATTGTGGCACACAGGGTATGCTAAAAGACCAAC |
| | | CCTCGAAAGAATCTGCCAATACTATATCTCCATCTGCCAG |
| | | TCTCTGTTTCTAA |
| SEQ | Solyc08g076730.4 | CAAAGTTTTCTCACTCAAATAATAAATATAATAAATAGTT |
| ID | | GTTTCAGTATGAAAACGGTCCTTTTCCGAACCGGTTCCGG |
| NO: | | CTCAATTCCGGCGGTTGTTCCCGGTTCTCAGAGAGCAGTC |
| 210 | | TCTGTTTCCACTCGTGAATCCATCGGTAAAGTGTTTAATG |
| | | GAGAGAAGAAGAAAGGATGTTCTTCTCCTATGATCTCTCT |
| | | GTATTCGGAAGTCAATCGTCGCGGTATTCGCCGGGTTTCA |
| | | TCTGACTCCGACGTGATCCGGTCGGGATTTGAAGGTTCGA |
| | | GTGTAACTAGAAAGTTGAGTAAGATTGGATCGAGGTCATT |
| | | ATCGTCAATAATACCGGAGGAGGATTGCGGTTCTGAACAG |
| | | AATGAGTTGGAGGTTTTGCGAAGTATTGGATCACTGAGTT |
| | | TGACGGAAGATCGACGGAGTTACGCTGGAGACTGGCATAG |
| | | GAACAGTATTCCAGTCGATGAGCTTGGATTTTCCGGCGGT |
| | | GGAATCGGTTCCACCAAAAACAAAAAGTTTGGCGGCAATG |
| | | GTGACGGTAGAGATGAGATCGATGCAGATAGTTTAACCGG |
| | | CGGAAAGTCAGATCCAAGGAAAATTGGAGCCTATTATAAG |
| | | GAAATGCTGAATTCAGATCCTATGAATCCTCTTCTTCTTA |
| | | GAAACTACGGCAAGTATTTGCATGAGGTGGAGAGAGACTT |
| | | TGTGAAGGCTGAAGAATGCTACGGAAGAGCTATACTTGCA |
| | | AGTCCTGGTGACGGAGAAGTGCTTTCTCTGTATGGCAAAT |
| | | TAGTTTGGGAGACGCAGCGAGACGAAAACAGAGCCAAATC |
| | | TTACTTTGATCAAGCTGTTCACGCTTCTCCTAATGACTGC |
| | | ACTGTGTTGGGATCATATGCACACTTCATGTGGGAAGCCG |
| | | AAGGAGACCAAGACGAAGACGAAGACGAAGAAAGAGAAAC |
| | | AGTAGCAGCTGGAGCATCAATGGTTGCAGCTTACTAG |
| SEQ | Solyc08g082620.3 | ATGAGTAGTATTACTTATTCTAATTTGATATTGAAGAGGA |
| ID | | GAGAATCAAGAATCAATGGTGGATCTCATTTACAATATAA |
| NO: | | TGGACTTAGAGCTATTGAAACTGTGAAATTGACAGCAAAC |
| 211 | | TCTGCTTCTTGTAAGCCAAAAGCTGTAAAATGTGGAAGAA |
| | | TCAGAGCCATGGCCTCTCCAGCTGTTTCAGCTCCCAAGAG |
| | | AGAGACAGACCCGAAGAAAAGGGTTGTCATAACCGGAATG |
| | | GGACTTGTTTCAGTCTTTGGAAGTGATATCGACAATTTTT |
| | | ACAACAAACTTCTTGAAGGGCAGAGTGGAATCAGTTTAAT |
| | | AGACTCTTTTGATGCCTCAAGTTACCCAGTTAGGTTCGCG |
| | | GGACAGATTCGTGATTTCTCTTCCGAAGGTTACATAGACG |
| | | GAAAGAATGATCGTCGTTTAGATAACTGCTGGAGATACTG |
| | | TCTCGTTGCTGGAAAGAGAGCCCTTGACGATGCTAACCTT |
| | | GGCCAACAAGTTCTTGAAACTATGGACAAAACGAGGATCG |
| | | GTGTCTTGGTTGGGTCGGGGATGGGAGGTTTAAATGTTTT |
| | | CAGTGACGGGGTGGAAGCGTTGTTGCAAAGAGGATACAAG |
| | | AAAGTTAGTCCATTTTTCATTCCTTACTCTATCACCAACA |
| | | TGGGATCAGCATTGTTGGCTATAGAAACCGGACTAATGGG |
| | | ACCGAATTACTCTATTTCAACGGCTTGTGCATCTGCCAAT |
| | | CACAGCTTTTGCGCAGCTGCGAATCATATAAGAAGGGGTG |
| | | ATGCAGATATTATGGTAACAGGTGGAACGGAAGCTGGTGT |
| | | TACAGCTACTGGTGTTGGTGGCTTCATAGCTTGTCGGGCC |
| | | TTGTCTCAAAGAAATGATGATTATGAAAAGGCTTCAAGGC |
| | | CATGGGACACGAATCGTGATGGATTTGTCGTTGGTGAAGG |
| | | CTCTGGTATCCTGGTCATGGAAAGTTTGGAGCACGCGTTG |
| | | AAAAGAGGTGCTAGTATCATCGCAGAGTACTTGGGAGGAG |
| | | CAGCTACTTGTGATGCTCATCACATGACCGATCCTCGGCC |
| | | TGATGGACTTGGAGTTTGGTCTTGCATTACTAAGGGTTTG |
| | | TTAGATGCCAGAATTTCTCCAGAAGAGGTGAACTATATAA |
| | | ATGCTCATGCAACATCAACTCTTGCTGGAGATTTGGCTGA |
| | | GGTCAATGCCATCAAGAAGGTCTTCAAAGATACCTCGGAA |
| | | ATCAAAATGAACGGGACAAAGTCGATGATCGGTCATGGAC |
| | | TTGGGGCTGCTGGTGGAATTGAAGCCATTGTAACCATCAA |
| | | AGCAATCACAACAGGATGGCTTCATCCAACCATCAATCAA |
| | | CATGAATTAGAACCCCAGGTCACGATTGACACCGTTCCAA |
| | | ATGTGAAGAAACAACATGAAGTGAACGTTGGTATCTCCAA |
| | | CTCGTTCGGATTCGGAGGACATAATTCAGTGGTTGTTTTT |
| | | GCACCTTACAGGCCTTGA |
| SEQ | Solyc09g008280.2 | ATGGAAACTTTCTTGTTTACCTCAGAGTCAGTCAACGAAG |
| ID | | GTCACCCCGACAAGCTCTGTGACCAGGTCTCAGATGCCAT |
| NO: | | TCTTGATGCTTGCCTGGAGCAGGATCCCGAAAGCAAGGTT |
| 212 | | GCATGTGAAACCTGCACAAAGACAAACATGGTTATGGTCT |
| | | TTGGAGAGATCACAACCAAGGCCACCGTAGACTACGAGAA |
| | | GATCGTACGTGACACATGCAGAGGCATTGGGTTCGTCTCA |
| | | GCAGATGTTGGTCTTGATGCTGACAACTGCAAGGTCCTTG |
| | | TTAACATTGAACAACAGAGCCCTGACATCGCCCAAGGAGT |
| | | TCACGGTCATCTTACCAAGAAACCAGAAGAAATTGGAGCT |
| | | GGTGACCAAGGTCATATGTTTGGGTATGCCACAGATGAAA |
| | | CTCCTGAGCTCATGCCCCTCACCCATGTTTTGGCCACCAA |
| | | GCTTGGTGCCAAGCTTACTGAAGTGAGGAAGAACAAAACC |
| | | TGCCCATGGCTCAGGCCCGATGGCAAGACCCAAGTTACTG |
| | | TTGAGTACAAGAACGACAATGGTGCCATGGTCCCTATTAG |
| | | AGTTCACACTGTTCTCATCTCAACCCAGCATGACGAAACT |
| | | GTCACCAACGACCAGATCGCCCAGGACTTGAAAGAGCATG |
| | | TGATCAAGCCTGTGATCCCAGCTAAGTACCTTGACGAGAA |
| | | CACCATCTTCCACCTCAACCCATCAGGTCGCTTCGTCATT |
| | | GGTGGTCCACATGGAGATGCTGGTCTTACTGGCAGGAAAA |
| | | TCATCATTGATACCTACGGAGGCTGGGGTGCTCACGGTGG |
| | | AGGTGCCTTCTCAGGAAAGGACCCCACTAAGGTGGACAGG |
| | | AGTGGTGCTTACATTGTTAGGCAGGCAGCAAAGAGTGTGG |
| | | TCGCCTCAGGACTTGCTCGCCGCTGTATTGTGCAGGTTTC |
| | | TTATGCTATCGGTGTGGCTGAACCACTTTCCGTGTTTGTT |
| | | GACACATACAAGACAGGAACAATTCCCGACAAGGACATTT |
| | | TGGTTCTGATCAAGGAGAACTTTGACTTCAGGCCTGGAAT |
| | | GATGTCAATCAACCTTGATTTGTTGAGAGGAGGCAACTAC |
| | | AGGTACCAGAAGACTGCAGCTTACGGTCACTTTGGCCGTG |
| | | ATGACCCCGATTTCACCTGGGAAACTGTCAAGGTCCTCAA |
| | | GCCAAAAGCTTGA |
| SEQ | Solyc09g009020.3 | ATGGCAACTATCAAATCCATTAAAGCTCGTCAGATCTTTG |
| ID | | ACAGCCGTGGTAACCCCACAGTCGAGGTTGATGTACATAT |
| NO: | | CTCAAATGGTGTGTTTGCCAGAGCTGCTGTTCCAAGTGGT |
| 213 | | GCATCCACCGGAATTTACGAAGCCCTTGAATTGAGGGATG |
| | | GAGGATCTGACTACCTCGGAAAGGGTGTTTCGAAGGCTGT |
| | | TAACAATGTCAACTCAATTATTGGCCCTGCTCTTGTTGGC |
| | | AAGGACCCAACTGATCAGACTGGTCTTGATAACTTCATGG |
| | | TTCATCAGTTAGATGGAACTCAAAATGAGTGGGGTTGGTG |
| | | CAAAGAAAAGCTTGGTGCAAATGCCATCCTTGCTGTGTCA |
| | | CTTGCTGTGTGTAAAGCTGGCGCTGCTGTCAGGAATGTGC |
| | | CATTGTACAAGCACATTGCTGATTTGGCTGGTAACAAGAA |
| | | GTTGGTATTACCAGTTCCTGCCTTCAATGTGATTAATGGT |
| | | GGATCTCATGCCGGAAACAAACTCGCAATGCAGGAATTTA |
| | | TGATCCTTCCTGTTGGAGCTGCTAATTTCAAGGAGGCCAT |
| | | GAAGATGGGCTGTGAAGTCTATCACCATTTGAAGGCTGTG |
| | | ATTAAGAAGAAATATGGTCAGGATGCCACAAATGTCGGTG |
| | | ATGAGGGTGGTTTTGCTCCTAATATCCAGGAGAACAAGGA |
| | | AGGTCTTGAATTGCTCAAGACAGCCATTGAGAAAGCAGGT |
| | | TACACTGGAAAGGTGGTCATTGGAATGGATGTTGCAGCAT |
| | | CTGAATTTTACGGAAAGGACAAATCTTATGACCTGAACTT |
| | | CAAAGAAGAGAGCAATGACGGCTCACAAAAGATATCAGGT |
| | | GACCAACTCAAGGACTTATACAAGTCATTTGTGTCCGAGT |
| | | ACCCTATTGTTTCAATTGAAGATCCATTTGACCAAGATGA |
| | | CTGGGAGACCTATGCTAAGCTCACTGCTGAGATTGGGGAG |
| | | CAAGTACAGATTGTCGGTGATGACCTTCTTGTCACCAACC |
| | | CTAAGAGGGTCGCCAAGGCAATTGCAGAGAAGACTTGCAA |
| | | TGCTCTTCTTCTCAAGGTTAACCAAATCGGTAGTGTGACC |
| | | GAGAGTATTGAAGCTGTGAAGATGTCCAAGAAGGCAGGTT |
| | | GGGGTGTAATGACCAGTCACCGCAGTGGAGAAACAGAAGA |
| | | TACCTTCATTGCTGATCTTGCTGTCGGTTTGTCAACGGGA |
| | | CAAATCAAGACTGGAGCTCCTTGCAGGTCAGAGCGTCTCG |
| | | CCAAGTACAACCAGCTGTTGAGGATCGAAGAGGAACTCGG |
| | | ATCAGAGGCTGTTTATGCAGGAGCAAGCTTCCGCAAGCCC |
| | | GTTGAGCCCTACTAA |
| SEQ | Solyc09g064330.2 | ATGGATGCTCAATCCAAATCAGACTCTTTTTCATCTTCAA |
| ID | | ACCTAAGAATCATTGTCCAAAACAACCCTTCAGAATCCCA |
| NO: | | ACTCTCTGAATTGGGCATAAAATCTTGGCCAAAATGGGCT |
| 214 | | TGCTCACCAGGGAAATACCAATTAAAATTTGATGCAGAAG |
| | | AAACATGTTATTTGCTAAGAGGAAAAGTGAAAGTGTATCC |
| | | AAAGAACACAATAGAGATGTCACCAGTGGAGTTTGGTGCT |
| | | GGGGATCTTGTCATTATTCCCAAAGGACTTTGTTGCACTT |
| | | GGGATGTAACCCTTCCTGTTGATAAACACTATAAGTTTCA |
| | | TTCTTCCTAA |
| SEQ | Solyc09g074240.1 | ATGTTCCCTTTGCTTCATCTCTTCATTTTTCTTTGCCTTT |
| ID | | CATTCTCTTATTGCTGTTATGGACAAATCATCAATTCTTC |
| NO: | | CTCTTCTTCATGTCCTCTCGATTTCACTTCACTTCGCAAA |
| 215 | | ATCATAGATCAAAATTCAAAAAGACCCACTTTGAATTCCA |
| | | CCGCACAGTGTCAGTACATTCGTCAGGGTTTACGTCTAGT |
| | | TCAATCGGAGTATCTCCGCCGTACCAACTCTTTCTTCCCG |
| | | CCTATGAGTTCCGCGGATTCTTGCTGGAACTCTTATCAGT |
| | | CTCTGGTCAACGATTATGTCCAAAACTTTGATATTCGGAG |
| | | TTCATGTGGGTTTCAAACAGCTTGGATCTCTCAGGGTTGT |
| | | ATGAATATCACAACCCGTTTCGAGTTCGAGTCTAAGGTAT |
| | | CCCCTGCTGCGATTACTAATATTGTTTCTTCCTGTAATCA |
| | | GTCTTTAGAGAACAATTCCCCTTGTGCTACTTGTACAACT |
| | | AGTTTATCTGGGCTTGCTTCGTTTCTACCGGGTCCTTCTG |
| | | TTGGAAATTTGTTCGATTGTGCGGCTTATCCTTCGGTTTA |
| | | TGCTGCTGCTTTTGCTAATCAGTTTGGACCTACTGATAAG |
| | | GGTACTGCAAAATGTTTGTTTCTTCTTGATGTTAGTGTTG |
| | | GTAGCTCAGGGAAGGGTAAGAAGAGTGTGGTTGTTATTGT |
| | | GGTGGTGGTGGTTTGTGTGGTGGTTTTCGCAGTTGTTGTG |
| | | TTTGGTTATTGGTTTATGTGGAGGAAGAGGAAAATTGGGT |
| | | TGGCAAAAAAGTGGAATACTAAGAGATTGGAGAGTAACTT |
| | | GAGTTCTCGTTTGGATTCGATTAGTGGAAGTACTACTTTG |
| | | ATTAGGTATAGTTTTGATGAGATTAAGGCGGCTACAAAGA |
| | | ATTTCTCTAGGTTGAATATAGTTGGGACAGGAGGGTATGG |
| | | GAATGTGTATAAGGGTGTGTTGCCGGGTGGGATTGAAGTA |
| | | GCATTGAAGAGGTTCAAGAATTGTTCGGTTGCTGGTGATG |
| | | CGAATTTCACTCATGAAGTGGAGGTTATTGCTAGTGTTAG |
| | | GCATGTGAATTTGGTGGCTTTGAGAGGGTACTGCACAGCG |
| | | ACAACCCCTTTTGAGGGTCACCAGAGGATCATTGTTTGTG |
| | | ATTTGATGAAGAATGGGAGTCTTCATGATCATTTGTTTGG |
| | | AACGAGACATGAAAAATTGAGTTGGGGTATTAGGCAGAAG |
| | | GTTGCAATTGGCACTGCTAGAGGATTGGCTTACCTGCATT |
| | | ATGGAGCACAACCGGGTATTATTCACAGGGATATTAAGGC |
| | | TAGTAATATACTTCTAGATGAGAGTTTTGAACCTAAGGTG |
| | | GCGGATTTTGGATTGGCTAAGTTCACTCCTGAAGGGATGA |
| | | CTCATTTGAGCACTCGTGTAGCAGGAACGATGGGGTATGT |
| | | AGCACCTGAGTATGCTTTGTATGGCCAATTGACTGAAAGG |
| | | AGCGATGTTTATAGCTTTGGAGTTGTGCTTCTTGAGCTTT |
| | | TGAGTGGAAAGAAAGCAATAATGGAGTTTAAGGATGGGCA |
| | | GCCGACGCTTGTGACTGATTGGGCCTGGTCATTGGTTAGG |
| | | GAAGGCAGAGCATTAGATGTTCTTGAAGACAGCATTCCGC |
| | | ATTTGGGGCCTCCAGAAGTTATGGAGAAGTATGTATTGGT |
| | | AGCTGTTCTTTGTTCTCATCCAGAATTGTACGCGAGGCCA |
| | | ACAATGGATCAGGTTGTGAACATGTTGGACGCTGAGATAC |
| | | CTGTTCCAACCATACCGGAGAGACCAATTTCTCTCATTGC |
| | | TGATCTTGATGACATTGAAAGGTCTGTTAGCAGTGGTGGT |
| | | AACTCAGGCAATCTGTCCACTGCTGCAGGCTATCAGCCTT |
| | | ACATATTTGAGCGCGAGACACCTCTGGCAGATAGTGATTC |
| | | TGTCAGGGCAAGAACATTGGATCAGAGATCGCGATAG |
| SEQ | Solyc09g075090.1 | ATGAGGAACATGCTGTTTGCTTCACTATATCCCGCCCGAC |
| ID | | TTCATTTCCTTAAAACCCTAATCTTCAAAATTTCCTCTTC |
| NO: | | TTCTACGCTTTCTGCTCTCGATCTTCCCAGTCGGTCGAGT |
| 216 | | AGGACACTTTATGTTGGCAATCTTCCTGGTGATGTTCGTG |
| | | AGCGTGAAGTGGAAGATCTCTTTTACAAGTATGGACCGAT |
| | | TGCACATATTGAGCTGAAAATTCCACCAAGACCCCCTGGT |
| | | TATGCTTTTGTTGAGTTTGAAGAGGCTCGTGATGCTGAAG |
| | | ATGCTATTCGTGGTCGTGATGGCTATGATTTTGATGGGCA |
| | | CCGTCTGCGGGTTGAGCTTGCACATGGCGGGCGTGGTAAC |
| | | TCATCATCAAATGATCGTTATGGTGGTGGCGGTGGCGGCC |
| | | GTGGTCAGCGTGGTGGAGGAGTGTCAAGACGTTCAGATTA |
| | | TCGAGTGTTAGTTACAGGATTACCCCATTCAGCATCGTGG |
| | | CAGGATCTCAAGGATCACATGCGCCGTGCTGGGGATGTTT |
| | | GTTTCTCCCAAGTTTTCCGTGACGGGAGTGGAACCACTGG |
| | | TATAATTGATTATACAAACTATGATGACATGAAATATGCT |
| | | ATCAAAAAGCTTGATGACTCTGAGTTTCGAAATGCCTTTT |
| | | CTCGTGCGACAATTCGAGTTAAAGAATATGACCGCAGTCG |
| | | AAGTCGCAGCCGCAGTCGCAGTCGCTCTTACTCCAGAGGG |
| | | AAGAGTGTTAGTCGAAGCCGAAGCCGTAGCCGAAGTAGAA |
| | | GCCGAAGCAAGAGCAAAAGCAAATCTCCTAAAGTTAAGTC |
| | | TTCAAAGCGCTCAAGATCTCGTTCAAGGTCTGTGTCTTCT |
| | | CAGTCTCGTTCTGGGTCAAAAGGACGTCCTGTCTCAAGAT |
| | | TTTGGATATGTGCGGCGGTTTGTGGATTTGCATATTAG |
| SEQ | Solyc09g092380.3 | ATGGCTCTACTCGTTGAGAAGACCACCTCTGGCCGCGAGT |
| ID | | ACAAGGTGAAGGACATGTCTCAGGCTGACTTCGGCAGGCT |
| NO: | | CGAAATCGAGCTTGCTGAAGTTGAAATGCCTGGTCTCATG |
| 217 | | GCTTCACGTGCTGAATTTGGCCCTTCACAGCCCTTTAAAG |
| | | GTGCAAAGATCACTGGATCTCTGCATATGACTATCCAAAC |
| | | TGCTGTCCTTATTGAAACCCTAACTGCTTTGGGTGCTGAA |
| | | GTTAGATGGTGTTCTTGCAACATCTTCTCAACTCAGGACC |
| | | ATGCTGCAGCAGCCATTGCACGTGACAGTGCTGCTGTCTT |
| | | TGCCTGGAAAGGTGAGACTTTGCAGGAGTACTGGTGGTGT |
| | | ACTGAGAGGGCACTTGACTGGGGTCCAGGTGGTGGTCCTG |
| | | ATCTGATTGTTGATGATGGAGGTGATGCTACTCTGTTGAT |
| | | TCATGAGGGAGTTAAGGCTGAAGAGGAGTTTGCTAAGAAT |
| | | GGAACAGTCCCAGATCCCACTTCTACTGACAATGTTGAGT |
| | | TTCAACTTGTGCTTACTATTATTAAGGAGAGCTTAAAGAC |
| | | TGATCCATTAAGGTACACTAAGATGAAGGAGAGACTTGTT |
| | | GGTGTTTCTGAGGAAACTACCACTGGTGTTAAGAGGCTTT |
| | | ACCAAATGCAGGCTAATGGATCTTTGCTTTTTCCTGCTAT |
| | | CAATGTTAATGACTCTGTTACCAAGAGCAAGTTTGACAAC |
| | | TTGTATGGATGCCGCCACTCACTTCCCGATGGTCTCATGA |
| | | GGGCTACTGATGTTATGATTGCTGGAAAGGTTGCTCTTGT |
| | | TGCTGGTTATGGAGATGTCGGCAAGGGATGTGCTGCTGCC |
| | | ATGAAACAAGCTGGTGCCCGTGTGATTGTGACTGAGATTG |
| | | ACCCAATCTGTGCTCTCCAGGCTACCATGGAAGGCCTCCA |
| | | GGTTCTTCCTCTTGAGGATGTTGTTTCTGAGGTTGATATC |
| | | TTTGTGACCACCACCGGTAACAAGGACATCATCATGGTTG |
| | | ACCACATGAGGAAGATGAAGAACAATGCCATTGTCTGCAA |
| | | CATTGGTCACTTTGACAACGAAATCGACATGCATGGTCTT |
| | | GAAACCTTCCCTGGTGTGAAGAGGATCACAATCAAGCCAC |
| | | AAACCGACAGATGGGTCTTCCCAGACACCAACAGTGGCAT |
| | | CATTGTGTTGGCCGAGGGTCGTCTCATGAACTTGGGATGT |
| | | GCCACTGGACACCCCAGTTTTGTGATGTCTTG |
| | | CTCTTTCACTAACCAAGTCATTGCCCAACTCGAGTTGTGG |
| | | AATGAGAGGAGCAGTGGCAAATACGAGAAGAAGGTGTACG |
| | | TCTTGCCAAAGCACCTTGACGAGAAGGTTGCTGCCCTTCA |
| | | TCTTGGAAAGCTCGGAGCCAAGCTTACCAAACTCACCAAG |
| | | GATCAAGCTGACTACATTAGCGTACCTGTTGAGGGTCCTT |
| | | ACAAGCCTGCTCACTACAGGTACTGA |
| SEQ | Solyc09g097960.3 | ATGACGAAAGAGGGAAAGAATGTGAAAAGAATCAAATTGG |
| ID | | GTTCAGAGGGACTTGAAGTATCTGTTCAAGGACTTGGTTG |
| NO: | | TCTGGGTATGTCCGATTTCTACGGTCCGCCCAAACCCGAA |
| 218 | | CCCGATATGATTGAACTAATACACCATGCCATTAACTCCG |
| | | GCATAACATTTTTCGATACTTCAAATAGTTATGGGCCTTA |
| | | CACTAATGAAATCCTCCTTGGCAAGGCTTTGAAGGGAGGA |
| | | ATGAGGGAACGAGTCGAGTTAGCAACAAAATTTGGTATAC |
| | | ATTTTGCAGATGGAAAGATAGAAGTACGTGGAGAGCCAGA |
| | | ATATGTAAGGGCAGCATGCGAGGCTAGCTTAAAGCGACTT |
| | | GATGTTGATTGCATAGACTTGTACTACCAGCACCGGATTG |
| | | ATACACGCGTGCCCATTGAAGTAACGGTTGGAGAACTTAA |
| | | GAAGCTGGTTGAAGAGGGTAAAATAAAATATATAGGTCTA |
| | | TCAGAGGCTTCAGCATCAACAATTAGAAGAGCACACGCTG |
| | | TTCATCCAATAACAGCAGTACAATTAGAATGGTCACTATG |
| | | GTCGAGAGATGTAGAGGAAGAAATAATCCCTACTTGCAGA |
| | | GAACTCGGAATTGGGATTGTAGCATACAGTCCACTAGGCC |
| | | GGGGCTTTTTGTCATCCGGTCCAAAGCTGGTTGAGGACTT |
| | | GACAAATGAAGATTTCCGAAAGCATCTACCGAGGTTTCAG |
| | | GGCGAGAATCTTGAGCATAACAAAAAATTGTACGAGAGGA |
| | | TTTGTGAATTGGCAACTAAGAAGGGATGCACCCCGTCTCA |
| | | ACTGGCGTTGGCGTGGGTACATCACCAAGGAAACGACGTG |
| | | TGTCCCATCCCGGGCACCACTAAGATTGAAAATTTCAATC |
| | | AGAACATTGGAGCTCTGTCCATAAAGCTAACATCAGAAGA |
| | | CATGGCAGAGCTTGAATCTATTGCTTCAGCTAATGCAGTC |
| | | CAGGGTGATAGGTACGGTTCGGCTGCAGCAACCTACAAAG |
| | | ATTCAGAAACTCCTCCTATTTCAGCATGGAAGGCTACATA |
| | | A |
| SEQ | Solyc09g098520.4 | ATGAAGAACGGTATGGCAGAATGCGCTGTCTGTCATTCCA |
| ID | | AGCTTGTTTCTCCATCTGTGAAAACTATTTCAAGGGCATA |
| NO: | | TGATCGACACAGAAGCAAGATTTCATCAAAGCAACGTGCT |
| 219 | | CTCAATGTCCTATTGGTTGTAGGAGATTGTATGCTGGTTG |
| | | GTTTACAGCCTGTTTTGGTATTTATGTCGAAAGTGGATGG |
| | | AAAATTCAAGTTTAGTCCCGTAAGTGTCAACTTCTTGACA |
| | | GAGGCGACAAAAGTCGTCTTTGCAATAATAATGCTTTTAA |
| | | TCCAGGCCAGGCATCAAAAGGTTGGAGAAAAGCCACTTCT |
| | | TTCAATATCCACATTTGTCCAGGCTGCAAGAAACAATGTC |
| | | CTTCTTGCTGTACCAGCACTACTCTATGCCATAAACAACT |
| | | ACCTAAAGTTCATCATGCAGTTATATTTCAATCCAGCAAC |
| | | AGTGAAGATGCTAAGCAATCTGAAGGTTTTGGTTATTGCA |
| | | GTGCTGTTGAAATTTATCATGAAAAGGCGATTTTCCGTAA |
| | | TTCAGTGGGAGGCTCTTGCTTTGCTGCTAATTGGGATAAG |
| | | CATTAATCAACTTCGATCCCTTCCTGAAGGCACCACTTCT |
| | | TTGGCTCTTCCAGTTACCACAATTGCCTACATCTATACAC |
| | | TGATTTTTGTAACTGTGCCTTCTATGGCCTCAGTATTCAA |
| | | TGAGTATGCTTTGAAGAGTCAATATGACACTAGCATATAT |
| | | CTCCAGAACTTATTTCTCTATGGTTATGGTGCTATATTCA |
| | | ACTTTCTAGCCATTCTTGGGATTGCTGTATTTAAAGGTCC |
| | | TGGTAGCTTGGATATTTTTCAGGGGCATTCGAAGGCAACC |
| | | ATGCTTCTAATTGTTAACAATGCAGCGCAAGGAATTCTGT |
| | | CGTCCTTTTTTTTCAAATATGCTGATACAATTCTGAAGAA |
| | | GTATTCTTCGACCGTTGCAACAATATTTACAGGCATAGCA |
| | | TCTGCTCTGCTGTTTGGTCATACACTGACCGTAAACTTTC |
| | | TTCTTGGGATTTCTGTTGTTTTCATTTCAATGCATCAGTT |
| | | CTTTTCACCTCTTTCAAAAGTTAAAGATGATCAACAAAAC |
| | | GGGACAGTGGAGTTGATTGACGTCCGGGAGAACCATAGCC |
| | | CTAAAGATTCATCCTTTGTAAATATGGCAGCAGGAGCAAA |
| | | TGAGGAGGCAAGTCATCGAGTAGGACCTGATGAAAGGCAG |
| | | CCACTTCTTCCAAGATGA |
| SEQ | Solyc10g005510.3 | ATGGCTTTTTCTTCTCTTCTCAAACCTGCCGCCTCCTTTG |
| ID | | TTCGACCTTCACATCGATCTCAGGTATCATGTGCAGGACT |
| NO: | | TCATCATAGTAGTAATTCTGTTAAATTACAATCGTCTATC |
| 220 | | TTCGGAGATGCTGTGACAATTTTGCAATCTTCGTCTTTAC |
| | | AAAAATCTGGTGCCTGCAGTATTCAACCTATCAGAGCAAC |
| | | TGCTACTGAGCTGCCTCCAACAGTTCCAAGGTCACAAACT |
| | | GGTGGGAAGACAAGGGTTGGCATCAATGGTTTTGGACGTA |
| | | TTGGGAGATTGGTATTACGAATTGCAACATTCAGGGATGA |
| | | TATTGAAGTGGTCGCAGTTAATGACCCATTTATCGATGCA |
| | | AAGTATATGGCTTACATGCTCAAGTATGATTCCACTCATG |
| | | GAGTCTACAGTGGATCCATCAGTGTCCTGGATGACTCTAC |
| | | TTTGGAAATCAATGGGAAGCAGATTAAAGTCAGTAGCAAA |
| | | AGGGATCCCGCAGATATTCCATGGGGTGATTTAGGTGCAG |
| | | ATTATGTTGTTGAATCTTCTGGTGTCTTCACAACTATTGA |
| | | TAAGGCATCAGCACATAAGAAGGGTGGTGCAAAAAAGGTC |
| | | GTAATCTCAGCACCATCAGCTGATGCACCTATGTTTGTGG |
| | | TAGGAGTGAATGAGAAAACTTACAAAGCCAACATGGATGT |
| | | TGTTTCTAACGCTAGCTGTACTACCAATTGCCTTGCTCCC |
| | | CTTGCCAAGGTGGTTCATGAAGAGTTTGGCATTGTTGAGG |
| | | GATTAATGACTACTGTGCATGCAACAACAGCTACCCAAAA |
| | | GACTGTTGATGGGCCATCAATGAAGGATTGGCGAGGAGGC |
| | | CGTGGTGCTGGACAAAACATCATTCCTAGTTCAACTGGTG |
| | | CTGCTAAGGCTGTAGGAAAAGTTCTGCCAGAATTGAATGG |
| | | GAAGCTCACTGGAATGGCTTTCCGTGTCCCAACACCTAAT |
| | | GTCTCTGTTGTTGACCTGACTTGCCGGCTAGATAAAAGTG |
| | | CTTCTTATGATGATGTGAAAGCAGCGATAAAGTATGCATC |
| | | GGAGGGTCCACTTAAAGGCATTTTGGGTTACACAGACGAG |
| | | GATGTTGTCTCAAATGATTTTGTTGGCGATTCCAGATCAA |
| | | GCATATTTGATGCTAAAGCTGGTATAGGCCTGAGCAAATC |
| | | ATTCGTGAAGCTCGTCTCCTGGTATGATAACGAGTGGGGT |
| | | TACAGCAACCGAGTATTGGACCTCATCGAGCACATGGCGT |
| | | TGGTTGCATCCATCCATTGA |
| SEQ | Solyc10g008420.3 | ATGGCGGGTAAGCAATTAGATGTCCCGAGGACAAGTGGTG |
| ID | | GTAACTTGAAAGAGCAATTAGTGAGGAGAACTTTGCAGAA |
| NO: | | TGTGAGGTCTCAAGGGCACATATATGTTGAGCTTAGAGAG |
| 221 | | GATGGGAAGAGGCTGATCTTTTTCTGCACGTTATGTCATT |
| | | CTCCATGTTATAGCGATTCTGTTCTGTTTAATCATTTGAA |
| | | AGGCAATCTCCATACTGAAATGCTTGCTGCTGCTAAAGCC |
| | | ACATTGTTGAAACCTAATCCTTGGCCCTTCAATGATGGTG |
| | | TACTTTTCTTTAATGATCCGGAGCAAGATAAGCAAGATAA |
| | | GCAATCACCTAATGTGAATGTTGGGAAAAGCAGACTGGTT |
| | | GACACTTGTCTTGAAGATGAAAGTAGCGTTGCTATTGTTG |
| | | AGTATGATGACAACTTGAGACATAATGAGGATACATATGT |
| | | TTCCGAGTATGAGTACGGCCTGCTTGATAGTGAGTTAATT |
| | | GGTAATGAAGAGAGTGATTACTTGGTTATTCCGGGTGTGT |
| | | TGTGTAAAGACGAATTATCTGATCTAGAAGTGAAACATAT |
| | | TGGTATTGGAAAAATTGCCGCTCGTATTAGTGTACGTGGC |
| | | ATTGACTCAAAAAGTATTCGTAGAATATGGTGTGAATGGT |
| | | TGGCAAAGAAGGATTCTGATGATATGGATACCTCTGTGGT |
| | | TCCAGATCATGATTTTGCGGTTGTTACATTTCCTTATAAC |
| | | TATAATTTGGGTAGGTCCCCGTTACTTGATGACAGATTTT |
| | | TGCTCCCTTCCAGTCCTTATTCAGAATCTGAGGAGACCAG |
| | | TGTTACTGGAAAGAGGAAGAGGAAGTCATTTTCAGACCCC |
| | | GAGGATTTCAGCGAGTCTCTTAGTAATCATTGTGATTCGT |
| | | CTGGGGAAGAATCTCAATCAACAAATAACTCAAATATGAA |
| | | ACTGATCTTGGGAACATGTGATGATCAACTAGTGAGTTCA |
| | | AGAATTATATCCAGCAAAACTATGAGGAGAGAGTTGAGGA |
| | | AACAGCAGCGAGTGGCTTCCGAGAGAATGTGTGATATCTG |
| | | TCAACAGAAAATGCTTCCTGGTAAGGATGTGGCAACACTT |
| | | CTGAGCTGGAAATCTGGAAAACTTATGTGCAGCAGTAGAA |
| | | ATATGTCTGGGGCATTTCATTTGTTTCACGTATCTTGCCT |
| | | CATTCATTGGATACTTCAATGTGAATTGCAAACCTCTGTG |
| | | AAACCTGTTGACGAACCAAAAATGGAACCAAAAGCAAAAC |
| | | GAAGATCTAAGAAGAAGACTGGAACCAAACACAATGCAAA |
| | | AGAGAAGGAAGATGAGACAAAATCAGCGAGACGGATCAAC |
| | | TCAGTGTTCTGTCCGGAATGCCAGGGCACTGGTATCTGCA |
| | | TCGAGGGAGATGAGCTGGAGAAGCCTCCTGTCTCACTTTC |
| | | TGAGGTGTACAGGCTCAAAATTAAACTTAGTGATGCACGA |
| | | AAAGCATGGATGAAGAATCCTGAGGTGTTGCAAAATTGCT |
| | | CAACAGGTTTTGATTTACCCCCTGAGCATGACGATCTTCT |
| | | CCAGGAATATGTATCACCACTCAAATTGCTGCACTTTTAT |
| | | CGCGCCAATGTTTCACACTGA |
| SEQ | Solyc10g081120.3 | ATGGAGTCAAGGCATTCCATTCATCACGTACTGCTTCTAG |
| ID | | TTTTGTTTGGTTTGTCTGCCCTGTGCCAATGCTCTGCTAC |
| NO: | | TGGGGTTGAAGCAAACCAGACAGCAGTACTGCTTGTGAAT |
| 222 | | GCATCCGAAGCATCAGCAAGGAGAATACCTGATACCCTTT |
| | | TTGGTATATTCTTTGAGGAGATCAATCATGCTGGTGCTGG |
| | | TGGATTGTGGGCTGAGCTTGTCAACAATAGAGGTTTTGAA |
| | | GGTGGAGGCCCAAACGTACCTTCAAATATTGATCCTTGGT |
| | | CTATCATTGGAGATGAGTCCAAAGTGATTGTATCAACAGA |
| | | CCGTTCATCATGCTTTGATCGGAATAAAATTGCAGTTCAA |
| | | GTGCAGGTGCTCTGTGACCATACAGGTGCCAATATCTGTC |
| | | CAGATGGAGGAGTTGGCATCTACAACCCGGGATTCTGGGG |
| | | CATGAATATTGAGCAGGGAAAGAGTTACAAACTAGTGCTT |
| | | TATGTTCGTTCCGAAGAATCAGTCAATGTATCTGTCGCTT |
| | | TAACTGGTTCAAATGGATTGCAAAAGCTGGCTGCTGCCAA |
| | | CATTGTAGCTGCTGATGTTTCAAGTTGGACGAAGGTGGAA |
| | | ATTTTGTTAGAAGCAACAGGAACAGATCCCAATTCAAGAC |
| | | TGGAATTGAGATCATCTAAGAAAGGTGTTATTTGGTTTGA |
| | | TCAAGTCTCATTAATGCCTACTGACACATACAAGGGGCAT |
| | | GGCTTCAGGAAAGATCTCTTTGGAATGCTTAAGGATTTAA |
| | | AGCCAGCATTCATTAGATTTCCAGGTGGATGTTTTGTTGA |
| | | AGGTGACTGGTTAAGAAATGCATTTCGGTGGAAAGAAACT |
| | | ATTGGACAATGGGAAGAGAGGCCCGGACATTTCGGTGATG |
| | | TTTGGAATTACTGGACTGATGATGGACTTGGACATTTTGA |
| | | GTTCCTGCAGCTTGCCGAAGACTTAGATTCACTGCCCGTG |
| | | TGGGTTTTCAACAATGGAGTCAGTCACCATGATCAAGTTG |
| | | ACACTTCCAGTATTTTACCTTTTGTGCAAGAGATATTAGA |
| | | TGGTCTTGAGTTTGCAAGAGGTGATCCTACTTCAACATGG |
| | | GGTTCTATTCGAGCCAAAATGGGACATCCAGAGCCTTTTG |
| | | ATTTGAGATATGTGGCTATCGGAAATGAGGATTGTGGAAA |
| | | AACACAATACCGTGGAAATTACCTCAAGTTCTATACGGCC |
| | | ATCAAAGATAAATATCCAGATATTAAAATAATCTCAAACT |
| | | GTGATGGTTCTACGAGACCACTGGATCACCCAGCTGATTT |
| | | ATATGATTTTCATATTTATAGCAGCGCAAGTTCTGTATTT |
| | | TCTAATGCTCGCCATTTTGATAGTGCACCACGCAGAGGAC |
| | | CAAAGGCTTTTGTGAGTGAGTATGCCGTGACTGGAAATGA |
| | | TGCTGGAAAAGGTAGTCTTTTAGCAGCATTGGGTGAAGCT |
| | | GGGTTCCTCATTGGGGTAGAAAAGAACAGCGAAGCAATTG |
| | | AAATGGCAAGTTACGCACCCCTATTTGTTAATGACAATGA |
| | | CCGGAGGTGGAACCCAGATGCAATTGTCTTCACCTCTTCA |
| | | CAGATGTATGGAACGCCTAGTTATTGGATGCAGCACTTCT |
| | | TCAAAGAGTCAAATGGCGCTACTCTTCTGAGTTCGTCATT |
| | | ACAAGCTAATCCTTCAAATTCACTTATAGCATCTGCCATC |
| | | ACTTGGCGAAATTCACTTGATAACAATGATTATTTGAGAA |
| | | TAAAGGTCGTGAACTTTGGAACCACTGCGGTTATAACTAA |
| | | AATCTCTCTCACTGGATTGGGGCAGAACTCGTTAGAGACG |
| | | TTATTTGGGGCTGTAATGACCGAGTTAACATCCAACAATG |
| | | TGATGGATGAAAATTCCTTCAGAGAGCCTAATAAGGTAAT |
| | | ACCTGTTAAAACACAAGTTGAGAAAGTTAGTGACAACATG |
| | | GATGTTGTACTAGCTCCAAGATCTCTGAATTCAATTGACT |
| | | TTTTATTAAGAAAATCAATAAACAACAATGTTGATACTGC |
| | | TTCTGTCCTTAAATCTTCATGCTAA |
| SEQ | Solyc11g006660.3 | AAAAAGTTAATCAAAGCCCTTGAATCTGCAAGAGGAAATG |
| ID | | GTACCAGTATGATTTCTCTTATTATTCCTCCTGGTGATCA |
| NO: | | GATATCTAGGATTACCAAGATGTTGGCGGAAGAATATGGT |
| 223 | | ACTGCATCAAATATTAAGAGCAGAGTGAATCGTCAGTCTG |
| | | TCCTTGGTGCAATTACGTCTGCCCAGCAGAGGCTTAAACT |
| | | GTATAATAAGGTACCTCCGAATGGTTTGGTCCTTTATACT |
| | | GGAACTATAATGACTGAAGATGGGAAGGAAAAGAAGGTAA |
| | | CCTTTGACCTTACACCTTTTAAGCCCATTAATGCGTCTCT |
| | | ATACCTATGTGACAACAAGTTTCACACGGGACCTCTGGGT |
| | | GAGCTTTTGGAATCAGATGAGAAGTTTGGTTTCATCGTCA |
| | | TGGATGGTAACGGTACTCTTTTTGGGACCTTAAGTGGCAA |
| | | CACTAGGGAAATCCTTCACAAATTTACTGTTGACCTTCCC |
| | | AAGAAGCATGGAAGAGGAGGTCAATCAGCTCTGCGATTTG |
| | | CTCGTCTTCGAATGGAGAAACGCCATAACTATGTGAGGAA |
| | | GACAGCAGAGCTTGCTACTCAGTTCTTCATTAATCCAGCC |
| | | ACTAGTCAGCCAAATGTATCTGGACTAATACTTGCTGGGT |
| | | CAGCTGATTTCAAGACGGAGCTGAGTCAGTCTGATATGTT |
| | | TGATCCACGTCTTCAGGCAAAGATACTTAATGTGGTTGAT |
| | | GTGTCCTATGGTGGGGAAAATGGATTCAGTCAGGCCATCG |
| | | AGCTATCTGCTGAGATTCTCTCGAATGTGAAGTTCATACA |
| | | AGAGAAGCGCTTGATAGGAAAATTCTTTGAGGAGATCAGT |
| | | CAAGATACTGGAAAGTATGTATTTGGTGTGGATGACACAA |
| | | TAAAAGCTCTGGAGATGGGAGCTGTTGAAACTCTTGTCGT |
| | | TTGGGAAAATCTTGATATAAACCGTTATGTGCTGAAAAAT |
| | | AGTGTTACCAATGAGATTGTCATTAAGCACCTAAACAAGG |
| | | ACCAAGAGGCTGATCAAAGCAACTTCAAGGATTCTGAAAA |
| | | TTCAGCTGAATTGGAGGTCCAGGACAAAATGCCGCTATTG |
| | | GAGTGGTTCGCCAATGAGTACAAAACCTTTGGTTGTTCAC |
| | | TTGAGTTTGTCACTAACAGGTCTCAAGAGGGGTCGCAGTT |
| | | CTGCCGAGGATTTGGTGGCATTGGGGGCATCCTTCGTTAC |
| | | CAGCTTGACATGCGTTCGTTTGATGAGCCATCTGATGAAG |
| | | GGGAATATTTTGAAGATTCTGATTAA |
| SEQ | Solyc11g006990.2 | ATGGCAAAGAATTCATTTTTATTACACATTTTGTTGTTTT |
| ID | | TTGTTCTTATTTTGAGTTTATGTGATGGGATTTTATCACA |
| NO: | | ATATGAGTTGGAGGAACTTACATGGTTAGATGATAAAGAT |
| 224 | | GATGAAATTTCAATGTTTCATAGCCAACATTCTTCTATGA |
| | | GGAGATGTGATTTTACTTCTGGGAAATGGGTTTTTGATCA |
| | | AAGTTATCCTCTATATGATTCAACTTGTCCTTATCTTAGT |
| | | ACTGCTGTTACTTGTACCAAAAATGGTAGACCTGATTCTG |
| | | ATTATGAAAAATGGAGGTGGAAACCTCATGGTTGTGAAAT |
| | | TCCAAGGTTTAATGCATTGGAATTTCTTGGAAGGATGAGG |
| | | AAAAAGAGAATTATGCTTGTTGGTGATTCTATAATGAGGA |
| | | ATCAATGGGAATCTCTTGTTTGCTTAGTTCAATCTGTTAT |
| | | CCCTATGGCTAGAAAAACAGTAACTTATGTTGGTCCTACT |
| | | ATGGCCTTCCATGCTATGGATTTCGAGACAACAATAGAAT |
| | | TCTGCTGGGCTCCTTTCTTGGTGGAACTGAAGAAAGGACC |
| | | AGAAAACAAGAGAATATTGCATCTGGACATGATTGAAGAG |
| | | AATGCAAAGTATTGGAGAGGAGCTGATGTTCTTGTTTTTG |
| | | ATTCAGCTCATTGGTGGACTCACTCCGATAAATATAGTTC |
| | | TTGGGACTTGATTATGGAAGGGAACAGTTTCTATAGAAAC |
| | | ATGAACCCAATGGTTGCCTATGAGAAAGGACTAATGACAT |
| | | GGGCTAAGTGGGTTGATTTGAATCTTGATCCTCGAAAAAC |
| | | TCGAGTTTTCTTCAGAAGCATGTCACCTCGACATAACAGG |
| | | GAAAATGGATGGAAATGCTTCAACCAGAGGGAGCCTCTGG |
| | | AGTTCTTTAGCCATCCGCATGTACCAGAACCACTGTTGGT |
| | | GCTAAAAGAGGTGCTACGAGGGATGAGTTTTCCGGTATCT |
| | | TTCCAGGATATAACAACAATGACAGCTCTTCGAAGAGACG |
| | | GGCATCCTTCTGTCTATAGTAAGTTTGTAAGCCAAACAGG |
| | | TAAGCAGCACTTAGGAGACTATAAATCTGATTGTAGTCAT |
| | | TGGTGCCTTCCTGGAGTACCTGATACCTGGAACGAAATGC |
| | | TGAATGTCATGCTCTAG |
| SEQ | Solyc11g011380.2 | ATGTCTCTGCTTTCAGATCTTATCAACCTCAATCTCTCAG |
| ID | | GTGATACTCAGAAGATCATTGCTGAATACATATGGATTGG |
| NO: | | TGGATCAGGCATGGACATGAGGAGCAAAGCCAGGACTCTC |
| 225 | | CCTGGTCCAGTTACTAGTCCTGCAGAACTACCCAAATGGA |
| | | ACTACGATGGATCGAGCACTGGTCAAGCTCCCGGAGAAGA |
| | | CAGTGAAGTGATCTTATATCCACAAGCAATCTTCAAGGAC |
| | | CCATTCAGAAGAGGCAACAACATCTTGGTCATGTGTGATG |
| | | CCTATACTCCTGCTGGTGAGCCCATCCCAACAAACAAGAG |
| | | GCACGCCGCCGCCAAGGTCTTCAGCCACCCTGATGTGGCT |
| | | GCTGAGGAAACTTGGTATGGTATTGAACAAGAATATACCT |
| | | TGCTGCAAAGGGAGGTCAACTGGCCTCTTGGATGGCCCAT |
| | | TGGCGGTTTTCCTGGCCCCCAGGGACCATACTACTGTGGA |
| | | ACCGGAGCTGACAAGGCCTTTGGACGTGACATTGTTGACG |
| | | CCCATTACAAGGCTTGTCTCTATGCTGGGATTAACATCAG |
| | | CGGGATCAATGGTGAAGTCATGCCGGGACAGTGGGAATTT |
| | | CAAGTTGGACCTTCTGTTGGCATCTCAGCTGGTGATGAAG |
| | | TGTGGGTAGCTCGTTACATTCTAGAGAGGATTGCAGAGAT |
| | | TGCTGGGGTGGTCGTGTCATTCGACCCCAAGCCTATTCCG |
| | | GGCGACTGGAATGGTGCAGGTGCTCACACAAATTACAGCA |
| | | CCAAGTCGATGAGGGAAGACGGAGGCTATGAAATAATCTT |
| | | AAAGGCTATTGAGAAGCTTGGCTTGAAGCACAAAGAACAC |
| | | ATAGCTGCATATGGTGAAGGCAACGAGCGTCGTCTCACTG |
| | | GAAAGCACGAAACAGCCAACATCAACACATTCAAATGGGG |
| | | GGTTGCAAACCGTGGTGCATCTGTCCGTGTTGGAAGAGAC |
| | | ACAGAGAAGGCAGGCAAGGGATACTTTGAGGACAGAAGGC |
| | | CAGCCTCAAATATGGACCCATACGTCGTTACCTCCATGAT |
| | | TGCAGAAACCACCATCATCGGTTAA |
| SEQ | Solyc11g012705.2 | AAGATGATCCAAGAAAATATTGTGATATTATTATCCTTAT |
| ID | | TATTAATTGTGCCAATAAGTTGGGCAATTAATAATCCATT |
| NO: | | AATAGAACTTTCAAGTAGAGAAGAATTGGCTAAGATAGCT |
| 226 | | GGTTATGGTGAAGAAAAACTCTCTACTGTTTTCCTACATG |
| | | GTACTCTTCTTTGTGGTCACACACCACATCCAGTTCCAGG |
| | | TGCATCTGTGGCGGTGTATTGTGGAACTAGTGGGCCGGGT |
| | | CGGGTAAGGAAATCATCATGGGCCAAAAATGTAACAAATG |
| | | AAAGTGGAGATTTTATAATAGATGTCCCTTCTCATCTCCA |
| | | TGCTAATATTCATGATCCAAATCTATGTCACATTCAAATT |
| | | CTTCACCTCCCAAAGGACTACTCACTTTGTCATCATCATT |
| | | CTACAAAAAGGAAAACATTCACACTGACATCGTCCAATAT |
| | | TGGCGTTCGCGTTTACACAACTCCAACAATTTACCTGACC |
| | | CCTAAAGCTAGCTCCCGAGTATAA |
| SEQ | Solyc11g040110.2 | ATGTTAGCGCATTGCACAAAGTCATCGCTGCCGGCGGCGG |
| ID | | ACGCCCACCTACCAACGCTGCGGCTCCCAAATCCACGGCG |
| NO: | | GAGTCCGGCCCCGGAGCTTCATTTCGGCTTCAATAGTTTT |
| 227 | | TCTACTAGAACATCGAAACTGCAAGTCAAAGCTTTGGACC |
| | | GGAACAGTAACAACAGTAGCACCGGTGACGGCGTCGGCGC |
| | | TAGTAAGAACGCCGCTTCATCGCCGTCATCATCGGTTCCG |
| | | AACACGAACTACGTGGTGCCACTAGACAAGGCTTCGTCTT |
| | | GCATTACTCGTCCCTTGGCCGAGATCCTACGTGACCTGAA |
| | | CAAGAGAATTCCTGATAACATCATCAAAGCTGGTAACAAT |
| | | GATGATCACTCCACCTTTATCACCTGGTACCATGCTAACC |
| | | GGATGTTGAGCTTCTACGCTCCAGGCTGGTGCGGGGAAGT |
| | | ACGAGATGTCATTTTCTCAGATAATGGAAGTGTGACAGTI |
| | | GTTTATCGTGTCACTATACGCGGATCAGATGGAGAGGCAC |
| | | ACCGTGAATCAACTGGAACAGTATCACCAGGCAATGGACA |
| | | GTACACAGATGTGGTTGCTGCTGCTGAGGAGATAGCATTT |
| | | TGCAGAGCATGTGCTCGTTTCGGGCTTGGCTTGTATCTGT |
| | | ATCATTAA |
| SEQ | Solyc11g066130.1 | ATGGCTTCTTCTTCTGCAAAAATTCTACTTCCCCTCTCTC |
| ID | | TCCTCTTCACCCTTTTATCTCTCTCCCAATCCACTAACCC |
| NO: | | TAATTTCATTTTAACCCTAGTTAACAACTGTCCTTACACC |
| 228 | | ATCTGGCCCGCAATTCAACCCAATGCCGGTCACCCTGTTC |
| | | TCGAACGAGGTGGATTTACCCTTCACTCACTTACCCACCG |
| | | CTCTTTCCCCGCTCCCAACGCTCACTGGTCCGGCCGAATC |
| | | TGGGCTCGTACCGGCTGCAATTACCAGCACGGAAAATTTT |
| | | ACTGCGCCACCGGAGACTGCGGCGGACGAATCGAGTGCGA |
| | | TGGACTCGGTGGTGCCGCCCCAGCTACATTAGCCCAGTTT |
| | | GTTCTACATCACGGACATGCCGATTTTTCTACTTACGGTG |
| | | TTAGTCTAGTGGATGGTTTCAACATTCCTCTTACTGTAAC |
| | | TCCACACGAAGGTAAAGGTGTTTGTCCAGTTGTTGGATGC |
| | | CGGGCGAATTTGTTAGAGTCCTGCCCAGCAGTTTTGCAGT |
| | | TTAGGTCACATGGTGGTCATGGACCTGTGGTTGGTTGTAA |
| | | AAGTGCTTGTGAAGCATTTAAATCGGATGAGTTTTGTTGT |
| | | AGGAATCATTATAATAGTCCACAGACCTGTAAACCATCGA |
| | | GTTATTCCCAGTTCTTTAAGCATGCTTGTCCTGCTACATT |
| | | CACATATGCACATGACAGTCCTTCACTTATGCATGAATGT |
| | | TCGTCTCCGCGTGAACTAAAGGTCATTTTCTGCCATTAA |
| SEQ | Solyc12g005140.2 | ATGATCGGTTGGTTTAAGCAGTTACATACTGCTTTTGGGT |
| ID | | TTTCCTTTCTATGGCTTGTTTGCCTCATTTACTTCACACA |
| NO: | | GGGGTTCAGATCTTTTGTCTGGACTGCAGTTTCATATCAG |
| 229 | | TTGAAAGACAACTTGAAGCTATCACCATCAGCCTCCCAAT |
| | | TTGTCACTTCAATAGCATTCTTTCCGTGGAGCATAAAACC |
| | | TTTATATGGGATATTATCTGATTGCTTTCCAATTAGAGGA |
| | | AGGAAAAGAGTCCCCTACCTTATGCTGGCAACTTTGCTTT |
| | | CGCTGTTGCCATGGCTCATTTTAGGGTTAAGTGATTTCCT |
| | | GAGAAATACGAGGACAGAACTCATGATTCTTTTGACACTG |
| | | CAGAACCTGGGTTCTGCAATGGCTGATGTTGTAATTGACG |
| | | CAATGATCGCTGAGGCAGTAAGATTTGAGAGGGCATCATT |
| | | TGCTGGTGATCTTCAGTCAGTATCTTGGCTGTCTATGGCA |
| | | TTCGGAGGAATCTGTGGTAGTTTGCTGGGAGGATATGCAT |
| | | TAGACAATATACGGATAGATATGATTTTCCTCCTTTTCTC |
| | | TGTTTTACCTGCTTTACAGCTCTTTTCGTGTGGTTTGGTT |
| | | GAGGAGGGTTCTGTACAAGGTAAAGCATTACCCGAAGGTT |
| | | CTGCATTAAATGGTTCTGATATGACGAGTGGAATTGTTGA |
| | | TGAAGAAAATAAATTCTCTGGTGAAAAATCAAAAGTTAGT |
| | | ACTTTGAGGAGAAAGAAGAGCCAGAATAATACAAAAAGAG |
| | | TGCCAACAACGGAAAGTAAATTTCAGGCTCCTGACAAAAA |
| | | GGGATCATCATTGTCTCCGTGGTATCAATCACTGAAAATG |
| | | GCCACTTCCACATTATTTAGGGCGTTCCGCCAGCCAGCCA |
| | | TTTTGAGACCAATGGCTTGGTTCTTTTTGGCTCATGTGAC |
| | | AGTTCCAAATCTCTCGACGGTTATGTTCTATTATCAGACA |
| | | GAGTTCTTAAAACTGGAGGCATCTTTCCTTGGAACAGCAC |
| | | GTGTTCTTGGATGGTTGAGTCTCATGATCGGGACCTTGAC |
| | | TTACAATCGCTATTTAAAAAGGATGAGGTTGCGGCATATT |
| | | CTCATGTTTACTCAAGTTTGCTTGGCCATTCTGACCATTC |
| | | TAGATATGGTTTTGGTTTCTCGAGCTAATGTTTCATTAGG |
| | | TATCTCAGACAAGGTTACAGTGCTATTTGGTTCTGCCCTT |
| | | TCTGATGGCATCAATCAATTCAAGTTCATGCCCTTTCTTA |
| | | TCTTATCGGGGCAGCTTTGTCCTCCAGGGGTTGAAGGAAC |
| | | TTTATTTGCATTGTTCATGTCAATAAACAACTTAGGTTCT |
| | | ACGGTGGGATCCTTTGTAGGAGCAGGGTTGGCTTCAGTTC |
| | | TAAACATCTCCTCAGGATCTTTTGACAATCTCTTCTTAGG |
| | | CATTGGTATACAGGTTCTCTGCACTTTCATCCCAGTGGCC |
| | | TTCCTTTTCTTGATTCCAAAGGAAGCTACAGGAATATCAG |
| | | CATAG |
| SEQ | Solyc12g088720.2 | ATGGCACAGATTCAGGTTCAGCATCAAACACCAGTGGCCG |
| ID | | GAGGAAATGGTGTGGCGGTTGCTGCTGCTGCAGCCGGTGC |
| NO: | | TGCCGCTGCTGTAGCGGCACCAGGTGGAGGAGCCACGAGT |
| 230 | | CAGTTTACGTCCACGTCGTTGTATGTTGGAGACTTGGACT |
| | | TTAACATCACTGATTCACAGCTCTATGATCTGTTCAACCA |
| | | TGTCGGTCAGGTTGTTTCTGTTAGGGTTTGTAGGGATCTT |
| | | AGTACTCGGAGATCTCTTGGATATGGATATGTTAACTACA |
| | | GTAACCCTTCCGATGCTGCAAGGGCAATGGAGTTGTTGAA |
| | | CTTCACTCCTGTGAATGGGAAGTCCATTAGGGTGATGTAT |
| | | TCTCATAGGGATCCCACTCTTCGTAAGAGTGGATCAGCAA |
| | | ATATATTTATTAAGAATTTGGACAAGTCGATTGACAACAA |
| | | AGCATTACATGACACCTTTTCAAGCTTTGGCAACATTCTT |
| | | TCTTGCAAGATAGCTACTGATTCTAATGGTCAGTCAAAGG |
| | | GTTATGGTTTTGTACAATTTGACAACGAAGAATCTGCTCA |
| | | AGGTGCCATAGATAAGTTAAATGGTATGTTGATGAATGAT |
| | | AAGCAAGTGTATGTTGGACACTTTCTTCGCAAACAGGAAA |
| | | GAGAATCTACAACTGGCATGACAAAATTCAACAATGTCTA |
| | | TGTGAAAAATTTGGCTGAATCTGCTACCGATGATGAGTTG |
| | | AAGAATGTTTTTGGTGAGTTTGGGACTATTACTAGTGCAG |
| | | TAGTGATGAGGGATGCAGATGGAAAATCCAAGGGCTTTGG |
| | | GTTTGTCAATTTTGAGAATGCAGATGATGCTGCTAAGGCT |
| | | GTTGAAGCATTAAATGGAAAGAAATTTGACGAAAAAGAAT |
| | | GGTATGTTGGGAAAGCACAGAAAAAATCTGAAAGAGAGCA |
| | | AGAATTGAAAAACAAGTTTGAGCAAACTGCCAAGGAGGCT |
| | | GTTGATAAATACCAAGGTGTTAATTTGTATGTAAAAAATT |
| | | TGGATGACACCATTGATGATGAAAAGCTCAAGGAACTTTT |
| | | TGCCGAGTTTGGTACTATTACCTCATGCAAGGTTATGCGG |
| | | GATCCAAGTGGAATCAGCAGGGGGTCTGGGTTTGTTGCTT |
| | | TCTCTACTCCTGAAGAAGCTTCCAGAGCTCTTTCTGAGAT |
| | | GAACGGCAAAATGATAGTGAGCAAGCCACTCTATGTTGCA |
| | | CTAGCACAGCGGAAAGAGGAGAGAAGGGCAAAGTTGCAGG |
| | | CACAATTTTCACAACTGCGACCAGTGGCAATGCCTCCATC |
| | | ACTTGCTCCTCGCATGCCAATTTACCCTCCTGGTGCTCCT |
| | | GGGATTGGACAGCAACTATTTTATGGGCAAGGACCCCCTG |
| | | CTCTAATTCCTCCCCAGGCTGGGTTTGGCTATCAACAACA |
| | | GCTTGTTCCTGGAATGCGCCCTGGAGGAGCCCCTATGCCA |
| | | AACTTCTTCATGCCGATGGTCCAGCAAGGACAACAGGGTC |
| | | AGCGCCCAGGTGGTCGACGAGGAGCAGGACCTGTGCAACA |
| | | ATCTCAACAGCCTATGCCCTTGATGCAGCAGCAGATGATG |
| | | CCAAGAGGAAGAATGTACCGCTATCCACCTGGACGCAATG |
| | | TGCCAGATGGGCCAATGCCAGGTGTTGCTGGTGGTATGCT |
| | | CTCTGTTCCATATGATATGGGTGGTATGCTTCCTCGTGAT |
| | | GCTGCTGCTATGGGACAGCCTGTGCCGATCTCTACATTGG |
| | | CTACTGCCCTTGCAAATGCTCCACCTGAGCAGCAGAGGAC |
| | | GATGTTGGGTGAAAGTTTGTACCCACTTGTTGATCAGCTA |
| | | GAGCACGAGCATGCTGCCAAGGTTACGGGCATGCTCCTTG |
| | | AAATGGACCAGACCGAGGTTTTGCATCTGCTTGAATCACC |
| | | GGAGGCTTTGAAGGCTAAAGTTTCCGAGGCTATGGATGTG |
| | | CTGAGGAATGTTCAGCAGGCAACCAGCAGCCCGGCTGACC |
| | | AACTTGCATCTCTTTCCCTTAATGACAACCTTGTTTCTTA |
| | | G |
| SEQ | Solyc12g095880.2 | ATGGGTACTGAGAGTAATTACCAGATGAAGGTGGTGAAAG |
| ID | | GAGATCATGGTTATGTTCTTGAAGATGTTCCTCATTTGAC |
| NO: | | TGATTATATCCCTGATCTTCCTACTCATGACAATCCATTG |
| 231 | | CGGTCCAATCCTGCATATTCAGTTGTGAAGCAGTACTTTG |
| | | TTGACATGGATGATACTGTCCCGCAAAAGGTTGTTGTTCA |
| | | CAAGGACAGTCCCAGAGGGGTGCATTTCCGACGTGCTGGT |
| | | CCACGTCAGAAGGTGTATTTCAATTCGGATGATGTTCGTG |
| | | CTTGTATTGTAACTTGTGGTGGTTTGTGCCCTGGGCTAAA |
| | | CACAGTGATCAGAGAGATTGTACATAGCCTTGATTATATG |
| | | TATGGGGTCAGCAAAGTCTTTGGGATCGATGGAGGCTACA |
| | | GGGGTTTCTATTCCAAGAATATCATCAATTTGACACCAAA |
| | | GACTGTTAATGACATTCATAAACGTGGTGGTACAATTCTT |
| | | GGATCATCACGAGGAGGCCATGATACCACAAAGATTGTTG |
| | | ACAGCATACAGGACCGTGGAATTAATCAGGTTTATATAAT |
| | | TGGTGGTGATGGAACTCAGAAAGGAGCAGCTGTAATATAC |
| | | GAGGAAATCAGGCGGCGTGATCTCAAAGTAATTGTTGCTG |
| | | GAATCCCAAAGACAATTGATAATGATATCCCTGTTATCGA |
| | | CAAGTCATTTGGTTTTGATACTGCTGTAGAGGAGGCTCAA |
| | | CGTGCCATAAATGCAGCTCATGTTGAAGCTGAAAGTGCTG |
| | | AAAATGGTATTGGTGTGGTGAAGCTAATGGGACGCTATAG |
| | | TGGATTCATCGCAATGTATGCCACTTTGGCGAGCAGAGAT |
| | | GTTGATCTCTGTCTAATTCCAGAGTCTCCATTTTATCTTG |
| | | AAGGAGATGGCGGACTCTTCGAATACATTGAAAAACGGCT |
| | | CAAAGAAAATGGGCACATGGTTATTGTGATAGCTGAAGGA |
| | | GCAGGGCAAGAACTTCTTGCAGAAGAGAATGCGCATGCCA |
| | | AAAACGAGCAAGATGCTTCGGGTAACAAGCTTCTTCAGGA |
| | | TGTTGGTTTGTGGATTTCCCAGAAAATCAGGGATCATTTT |
| | | GCTAAAAAAACTAAGATGCCCATTACTCTTAAATATATAG |
| | | ATCCGACTTACATGATTCGTGCTGTTCCAAGTAATGCCTC |
| | | TGATAATGTATATTGCACTCTTCTTGCTCAAAGTTGTGTT |
| | | CATGGAGCAATGGCAGGATACACAGGTTTCACCTCAGGAC |
| | | TTGTCAATGGTCGCCAGACTTATATTCCATTCAATCGTAT |
| | | TACCGAGAAACAAAATATGGTGGTTATAACTGACAGGATG |
| | | TGGGCACGTCTTCTTTCGTCAACCAATCAGCCAAGCTTCT |
| | | TGCGCGTGAAAGACATTGAAGAGATTAAAAAGGAGGAGCA |
| | | GCCGCAAACTCAATTATTGGACGGGGATAACAATGTACAT |
| | | GAGAACTCAGGTCACTGA |
| SEQ | Solyc 12g099970.2 | ATGGGGAATGCGAACGCCAGAGAAGACGGCGCCGCCGGAG |
| ID | | ACGGCGACGGCGACGGTCAGGTATCGGGAAGAAGATCTAA |
| NO: | | TGTTGAATCTGGAATAGTAGAAGATCACCACGCTCTGAAC |
| 232 | | TCGCGAGTGCCTTCGGCTGACTTGATGGTCAATTCTCCTC |
| | | CGCAGAGTCCACATCGTTCAGCTTCACCTCTCTTGTTCGG |
| | | ACCTCAGGTGCCAGTAGTTCCTTTACAAGGAGGGGATGGC |
| | | AATCCTGTTAGCAATCAAATGTGGGGCAATGAATGCGAGG |
| | | ATGCCTCTGATCATTCACTAGAGGGCGGCATACCTACTTT |
| | | GATAACATGGAGTTATGGTGGTAATAACGTTGCTATCCAA |
| | | GGATCTTGGGACAACTGGACATCAAGGAAAATTCTCCAAA |
| | | GATCAGGCAAGGACTATACCGTTCTTTTGGTCCTTCCATC |
| | | GGGTATATATCATTACAAATTCATTGTGGATGGGGAAGTT |
| | | AGATATATTCCAGAACTTCCATGTGTAGCAGATGAGACAG |
| | | GCGTTGTCTTTAATCTTCTTGATGTTAATGACAATGTTCC |
| | | AGAGAACCTCGAAAGTGTTGCAGAGTTTGAGGCCCCACCA |
| | | TCACCTGACTCTAGCTATGCGCAAGCTTTGATGGGAGAGG |
| | | AGGATTTTGAAAAGGAGCCAGTGGCAGTTCCACCGCAACT |
| | | TCATCTAACTGTTCTTGGTTCTGAAAACTCAGAAGAAGCA |
| | | CCTTCTTCTCCAAAACCCCAGCACGTAGTACTTAATCACC |
| | | TCTTTATAGAGAAAGGATGGGCTTCTCAATCCGTTGTTGC |
| | | TCTTGGTTTAACGCATAGATTCCAGTCCAAATATGTCACA |
| | | GTTGTTCTCTACAAGCCACTGAAGAGGTGA |
| SEQ | Solyc05g054040.4 | ATGTGCACAAAATTCGTGAAGGAAATGGAGAAGATGGCGT |
| ID | | TGTACGTCGTCGTTTTGCTAATCATCTGCTATGCATCGCC |
| NO: | | GGCGAGATCAGACGGTTCCGATCATAAGTATAAATCCGGA |
| 233 | | GATCAGGTTCCACTTTATGCTAACAAAGTCGGTCCGTTCC |
| | | ATAATCCGAGTGAAACATACCGCTTCTTTGACCTTCCTTT |
| | | TTGTACTCCAGATCATGTAAAAGAGAAGAAGGAAGCCCTT |
| | | GGTGAGGTCTTGAATGGTGACCGATTGGTCAGTGCCCCTT |
| | | ACAAGCTTGACTTTTTGTATGATAAGGACTCAGAAATAGT |
| | | TTGCAAGAAGAAGTTGTCCAAGGAAGAAGTTTCTCAATTC |
| | | CGCAGCGCTGTTGCTAAAGACTACTACTTCCAAATGTATT |
| | | ACGATGACCTGCCCATTTGGGGTTTCTTAGGGAAAGTTGA |
| | | TAAGGAAGGAAAGTCTGACCCCAGCGAGTACAAATACTAC |
| | | TTGTTTAAACATCTTCATTTTGAGATCTTTTACAATGATG |
| | | ATCGTGTGATTGAGATCAATGCACGAACTGATCCTAATGC |
| | | CCTGGTAGATATTACAGAGGATAAGGAGGTTGATGTTGAC |
| | | TTCATGTATACTGTAAAATGGAAAGAGACAAAAACACCTT |
| | | TTGATAAGAGGATGGAGAAGTACTCACAGTCTTCCTCGTT |
| | | GCCGCATCACTTGGAAATCCATTGGTTCTCAATTATCAAT |
| | | TCTTGTGTGACAGTTCTTCTCTTAACTGGTTTTCTTGCCA |
| | | CAATTCTTATGCGGGTCCTTAAGAATGACTTTGTCAAGTA |
| | | TGCACATGATGAGGAGGCGGCTGATGACCAAGAAGAAACT |
| | | GGGTGGAAATACATCCATGGTGATGTGTTTAGGTACCCAA |
| | | GTCACAAATCCTTGATGGCTGCAGCACTTGGGAGTGGCAC |
| | | ACAGTTATTCACATTGACCATCTTTATTTTCCTTCTCTCA |
| | | CTTGTTGGTGTTTTCTATCCATACAACCGTGGAGCTTTAT |
| | | TCACGGCACTGGTCGTCATATATGCTCTCACATCTGGTAT |
| | | TGCAGGCTATACTGCTGCCTCTTTCTATTGCCAGCTAGAA |
| | | GGGACAAACTGGGTCAGGAATTTGATATTGACCGGAGCCT |
| | | TGTTTTGTGGACCCCTGTTTCTCACGTTCTGTTTCCTTAA |
| | | CAGCGTAGCAATTGCATACAGTGCTACTGCTGCACTGCCT |
| | | TTTGGAACCATTGTGGTCATCTTTCTCATATGGGCTCTTG |
| | | TGACATCACCTTTACTTGTATTGGGAGGGATAGCTGGAAA |
| | | GAACAGCAGGGCTGAGTTCCAAGCTCCTTGTCGAACCACC |
| | | AAATACCCAAGAGAGATCCCACCATTACCTTGGTACCGCG |
| | | GAACTCTACCTCAGATGGCAATGGCTGGGTTTTTGCCTTT |
| | | CAGTGCCATATATATCGAACTTTACTATATATTTGCAAGT |
| | | GTTTGGGGTCACAGAATTTACACCATATACAGCATCTTAT |
| | | TTATAGTCTTCATCATTCTTATAATTGTGACTGCTTTTAT |
| | | TACCGTGGCCTTGACATACTTTCAACTTGCTGCTGAAGAT |
| | | CATGAATGGTGGTGGAGGGCTTTCCTTTGTGGTGGATCAA |
| | | CTGGACTGTTCATCTACGGATACTGCTTGTATTACTATTA |
| | | TGCACGTTCCGATATGACAGGGTTCATGCAAACCTCATTC |
| | | TTCTTCGGGTACATGGCTTGCATCTGCTATGGTTTCTTTC |
| | | TCATGCTCGGGTCTGTTGGTTTCCGTGCAGCCCTATTCTT |
| | | TGTCCGTCACATATATCGATCGATCAAATGCGAGTAA |
| SEQ | Solyc11g011910.2 | ATGTGCACAAAATTCGTGAAGGAAATGGAGAAGATGGCGT |
| ID | | TGTACGTCGTCGTTTTGCTAATCATCTGCTATGCATCGCC |
| NO: | | GGCGAGATCAGACGGTTCCGATCATAAGTATAAATCCGGA |
| 234 | | GATCAGGTTCCACTTTATGCTAACAAAGTCGGTCCGTTCC |
| | | ATAATCCGAGTGAAACATACCGCTTCTTTGACCTTCCTTT |
| | | TTGTACTCCAGATCATGTAAAAGAGAAGAAGGAAGCCCTT |
| | | GGTGAGGTCTTGAATGGTGACCGATTGGTCAGTGCCCCTT |
| | | ACAAGCTTGACTTTTTGTATGATAAGGACTCAGAAATAGT |
| | | TTGCAAGAAGAAGTIGTCCAAGGAAGAAGTTTCTCAATTC |
| | | CGCAGCGCTGTTGCTAAAGACTACTACTTCCAAATGTATT |
| | | ACGATGACCTGCCCATTTGGGGTTTCTTAGGGAAAGTTGA |
| | | TAAGGAAGGAAAGTCTGACCCCAGCGAGTACAAATACTAC |
| | | TTGTTTAAACATCTTCATTTTGAGATCTTTTACAATGATG |
| | | ATCGTGTGATTGAGATCAATGCACGAACTGATCCTAATGC |
| | | CCTGGTAGATATTACAGAGGATAAGGAGGTTGATGTTGAC |
| | | TTCATGTATACTGTAAAATGGAAAGAGACAAAAACACCTT |
| | | TTGATAAGAGGATGGAGAAGTACTCACAGTCTTCCTCGTT |
| | | GCCGCATCACTTGGAAATCCATTGGTTCTCAATTATCAAT |
| | | TCTTGTGTGACAGTTCTTCTCTTAACTGGTTTTCTTGCCA |
| | | CAATTCTTATGCGGGTCCTTAAGAATGACTTTGTCAAGTA |
| | | TGCACATGATGAGGAGGCGGCTGATGACCAAGAAGAAACT |
| | | GGGTGGAAATACATCCATGGTGATGTGTTTAGGTACCCAA |
| | | GTCACAAATCCTTGATGGCTGCAGCACTTGGGAGTGGCAC |
| | | ACAGTTATTCACATTGACCATCTTTATTTTCCTTCTCTCA |
| | | CTTGTTGGTGTTTTCTATCCATACAACCGTGGAGCTTTAT |
| | | TCACGGCACTGGTCGTCATATATGCTCTCACATCTGGTAT |
| | | TGCAGGCTATACTGCTGCCTCTTTCTATTGCCAGCTAGAA |
| | | GGGACAAACTGGGTCAGGAATTTGATATTGACCGGAGCCT |
| | | TGTTTTGTGGACCCCTGTTTCTCACGTTCTGTTTCCTTAA |
| | | CAGCGTAGCAATTGCATACAGTGCTACTGCTGCACTGCCT |
| | | TTTGGAACCATTGTGGTCATCTTTCTCATATGGGCTCTTG |
| | | TGACATCACCTTTACTTGTATTGGGAGGGATAGCTGGAAA |
| | | GAACAGCAGGGCTGAGTTCCAAGCTCCTTGTCGAACCACC |
| | | AAATACCCAAGAGAGATCCCACCATTACCTTGGTACCGCG |
| | | GAACTCTACCTCAGATGGCAATGGCTGGGTTTTTGCCTTT |
| | | CAGTGCCATATATATCGAACTTTACTATATATTTGCAAGT |
| | | GTTTGGGGTCACAGAATTTACACCATATACAGCATCTTAT |
| | | TTATAGTCTTCATCATTCTTATAATTGTGACTGCTTTTAT |
| | | TACCGTGGCCTTGACATACTTTCAACTTGCTGCTGAAGAT |
| | | CATGAATGGTGGTGGAGGGCTGTCATTCGTTTGTGGTGGA |
| | | TCAACCGGCTTGTTCATCTATGGCTACTGCCTGTATTACT |
| | | ATTATGCCCGATCAGACATGTCAGGTTTCATGCAAACCTC |
| | | ATTCTTCTTTGGTTACATGGCTTGCATTTGCTATGCCTTC |
| | | TTTCTCATGCTTGGAACTGTTGGTTTCCGAGCTGCTTTGT |
| | | TTTTCGTCCGCCACATATACCGTTCAATCAAGTGCGAGTA |
| | | A |
| SEQ | Solyc09g092390.2 | ATGGCTCTACTCGTTGAGAAGACCACCTCTGGCCGCGAGT |
| ID | | ACAAGGTGAAGGACATGTCCCAGGCTGACTTCGGCAGGCT |
| NO: | | CGAAATCGAGCTTGCCGAAGTTGAAATGCCTGGTCTCATG |
| 235 | | GCTTCCCGTGCTGAATTTGGCCCTTCACAGCCCTTTAAAG |
| | | GTGCAAAGATCACTGGATCTCTGCATATGACTATCCAAAC |
| | | TGCTGTTCTTATTGAAACCCTAACTGCTTTGGGTGCTGAA |
| | | GTTAGATGGTGTTCTTGCAACATCTTCTCAACTCAGGACC |
| | | ATGCTGCAGCAGCCATTGCACGTGACAGTGCTGCTGTTTT |
| | | TGCCTGGAAAGGTGAGACTTTGCAGGAGTACTGGTGGTGT |
| | | ACTGAGAGGGCACTTGATTGGGGTCCAGGTGGTGGTCCTG |
| | | ATCTGATTGTTGATGATGGAGGTGATGCTACTCTGTTGAT |
| | | TCATGAGGGTGTTAAGGCTGAAGAGGAGTTTGCTAAGAAT |
| | | GGAACAATCCCAGATCCTAGTTCTACTGATAATGTTGAGT |
| | | TTCAACTTGTGCTTACTATTATTAAGGAGAGTTTAAAGAC |
| | | TGATCCTTTAAAATACACTAAGATGAAGGAGAGACTTGTT |
| | | GGTGTTTCTGAGGAAACTACCACTGGTGTTAAGAGGCTTT |
| | | ACCAAATGCAGGCTAATGGAACTTTGCTTTTCCCTGCTAT |
| | | TAATGTTAATGACTCTGTTACCAAGAGCAAGTTTGACAAC |
| | | TTGTACGGATGCCGCCACTCACTTCCCGATGGTCTCATGA |
| | | GGGCTACTGATGTTATGATTGCTGGAAAGGTTGCCCTTGT |
| | | TGCTGGTTATGGAGATGTCGGCAAGGGATGTGCTGCTGCC |
| | | ATGAAACAAGCTGGTGCACGCGTTATTGTGACTGAGATTG |
| | | ATCCAATCTGTGCTCTCCAGGCTACCATGGAAGGTCTCCA |
| | | GGTTCTTCCTCTTGAGGATGTTGTTTCTGAGGTTGATATC |
| | | TTTGTGACCACCACTGGTAACAAGGACATCATCATGGTTG |
| | | ACCACATGAGGAAGATGAAGAACAATGCCATCGTCTGCAA |
| | | CATTGGTCACTTTGACAATGAAATCGACATGCATGGTCTT |
| | | GAGACCTTCCCTGGTGTGAAGAGGATCACAATCAAGCCTC |
| | | AAACCGACAGATGGGTTTTCCCAGACACCAACAGTGGCAT |
| | | CATTGTGTTGGCCGAGGGTCGTCTCATGAACTTGGGATGT |
| | | GCCACAGGACACCCCAGTTTTGTGATGTCTTGCTCTTTCA |
| | | CTAACCAAGTCATTGCCCAACTCGAGTTGTGGAATGAGAA |
| | | GAGCAGTGGTAAATACGAGAAGAAGGTATACGTTTTGCCA |
| | | AAGCACCTTGACGAGAAGGTTGCTGCCCTTCATCTTGGAA |
| | | AGCTCGGAGCCAAACTTACCAAACTTACCAAGGATCAAGC |
| | | TGACTACATTAGCGTACCAGTTGAGGGTCCTTACAAGCCT |
| | | GCTCACTACAGGTACTGA |
| SEQ | Solyc01g096590.3 | ATGGCAGCATATGCAGCAATGAAGCCAACCAAGCCTGGGC |
| ID | | TTGAGGAGCCTGCAGAGATGATTCACAAGATTAGGATCAC |
| NO: | | TCTCTCCTCCAAGAACGTTAAGAATCTCGAGAAAGTGTGC |
| 236 | | GCTGATTTGGTTCGTGGTGCCAAGGATAAGAGGCTCAGGG |
| | | TGAAGGGACCAGTGAGAATGCCCACAAAGGTCCTTAACAT |
| | | CACCACTAGAAAGTCTCCTTGTGGAGAAGGTACAAATACA |
| | | TGGGACAGATTTGAGCTTCGTGTCCACAAGCGGGTGATTG |
| | | ACCTTTTCAGCTCACCAGATGTTGTGAAACAAATCACTTC |
| | | AATCACCATTGAACCCGGTGTTGAGGTTGAGGTCACTATT |
| | | GCGGAATCTTAG |
| SEQ | Solyc05g014470.3 | ATGGCAAACGGAAAGATCAAAATCGGAATCAACGGATTCG |
| ID | | GTAGAATTGGTCGTTTGGTGGCTAGAGTTGCTCTACAGAG |
| NO: | | AGATGATGTTGAACTAGTTGCAGTGAATGATCCATTTATT |
| 237 | | TCCACTGATTACATGACATATATGTTTAAGTATGATTCAG |
| | | TACATGGACAATGGAAGCATCATGAGCTAAAGGTCAAGGA |
| | | TGAGAAGACACTTCTCTTTGGAGAGAAGGCTGTTACAGTT |
| | | TTTGGAATCAGGAACCCTGAAGATATCCCATGGGGTGAAG |
| | | CTGGTGCTGACTTCGTTGTTGAATCAACCGGTGTCTTCAC |
| | | TGACAAGGACAAGGCTGCTGCTCACTTGAAGGGTGGTGCC |
| | | AAGAAGGTTGTGATCTCTGCTCCTAGCAAAGATGCTCCCA |
| | | TGTTTGTTGTGGGTGTCAACGAGAATGAATACAAGCCAGA |
| | | GCTGGACATTGTCTCCAATGCTAGTTGCACAACGAACTGC |
| | | CTTGCACCTTTGGCTAAGGTTATCAATGATAGGTTTGGCA |
| | | TTGTTGAGGGTCTCATGACCACTGTCCACGCCATGACTGC |
| | | CACCCAGAAAACTGTTGATGGTCCATCCATGAAGGACTGG |
| | | AGAGGTGGAAGAGCTGCTTCATTCAACATCATCCCTAGCA |
| | | GCACTGGTGCAGCCAAGGCTGTTGGAAAAGTGCTCCCACA |
| | | ACTTAACGGCAAATTGACTGGAATGGCCTTCAGAGTACCA |
| | | ACTGCTGATGTCTCCGTTGTCGATCTTACTGTAAGACTCG |
| | | AGAAAGAAGCCTCCTATGAAGACATTAAGGCTGCAATCAA |
| | | GGAGGAATCAGAGGGTAAATTGAAGGGTATCTTGGGATAC |
| | | ACTGAAGATGATGTGGTTTCCACAGACTTTGTTGGTGACA |
| | | GCAGGTCAAGCATTTTTGATGCCAAGGCTGGAATTGCTTT |
| | | GAGCAAGAATTTTGTGAAAGTTGTGTCATGGTATGACAAC |
| | | GAATGGGGTTACAGTTCCCGTGTGATTGATTTGATCTGCC |
| | | ATATGGCTAAGGCTTGA |
| SEQ | Solyc05g007950.4 | ATGGCTTCTAATTCAGCCTTTTCTCTGTTCTTGATTTTGT |
| ID | | TAATTATTACACAATGTTTATCAGTCCTCAATGCTGCTAA |
| NO: | | AGATTTTGACTTTTTCTACTTTGTTCAACAGTGGCCAGGG |
| 238 | | TCATACTGTGACACAAAACAAAGTTGTTGTTACCCTACAA |
| | | CTGGAAAACCAGCAGCAGATTTTGGAATTCATGGACTTTG |
| | | GCCTAATAATAATGATGGGACTTATCCATCAAATTGTGAT |
| | | CCAAACAGTCCTTATGACCAATCTCAGATTTCTGACTTAA |
| | | TTAGTAGTATGCAACAAAATTGGCCAACACTAGCGTGCCC |
| | | AAGTGGTAGTGGCTCAACATTTTGGTCACATGAATGGGAA |
| | | AAGCATGGCACTTGTGCTGAATCCGTTCTCACAAACCAAC |
| | | ACGCTTATTTTAAGAAGGCTCTTGATCTCAAGAATCAAAT |
| | | TGATCTTTTGTCAATTCTTCAAGGTGCTGACATTCATCCT |
| | | GACGGCGAATCTTATGACCTGGTCAATATTAGAAATGCAA |
| | | TTAAAAGCGCGATTGGATATACTCCTTGGATCCAATGTAA |
| | | TGTAGACCAGTCGGGTAACAGTCAGCTATACCAGGTTTAC |
| | | ATTTGTGTTGATGGCTCGGGTTCAAGTCTCATTGAGTGCC |
| | | CTATTTTCCCTGGAGGGAAATGTGGCACAAGCATTGAGTT |
| | | CCCAACATTTTAA |
| SEQ | Solyc02g087540.3 | ATGGTAGATACATTGACTGAATTATGCTCCAAACTTGCTG |
| ID | | GACTGATGTTCATTTTGGGTACAATTCAGCAACTTGTCCC |
| NO: | | TCATTCTTTCTACAAGCGCATCAAAACATTGTGGCATCGA |
| 239 | | ATAGAAAATTATTTCTACCCTTATGTCCAAATTACCATCG |
| | | ATGAATACTCCAACTATAAAAGAAATGAAGTTTACTCTCT |
| | | TATAAAAGTTTATTTAGGTACTAAGTCCACTAACGATGCA |
| | | AAGTATCTTAAAGCTGAAATGTTCAAGAAGAGCAAGTTTT |
| | | TAGCTGTTAGCTTGGATGAAGGAGAAGAGGTAATTGATGA |
| | | ATTTCATGGTGTGAAGCTCAAATGGAACTCTTACACGGAA |
| | | ACATTCCCTGATAATTCTTCAGGGGGGCGTTCTCAACTTC |
| | | CGATTGAAAAGAAATGTTATACGCTAACTTTCAATAAGAA |
| | | AAATCAAGAGATGGTCACCGGACAATACTTGAAACATGTG |
| | | ATGGAAGAAGGGAAGGCCATTGAGTTCAAGAACAAGAAGC |
| | | AGAAGATCTACTCTAACGAAGAAGGGGAGAGGTATTGGTA |
| | | TGGTAAAGGTATGTGGAGGAACATAAATTTCGAGCACCCT |
| | | GCAACATTTGATACACTGGCTATGGATCCTACGAAGAAGG |
| | | AAGAAATAATCAATGATCTCGTTGCTTTCAGCAAGGGGAA |
| | | GGACTACTATTCCAAGGTTGGAAAGGCGTGGAAGCGCGGT |
| | | TATCTTCTTTATGGTCCACCAGGGACTGGAAAATCAACTA |
| | | TGATTGCAGCAATTGCGAATTACTTGAACTATGATATTTA |
| | | TGATCTCGAGCTCACCTCAGTTAAGGATAACTCAGAGCTT |
| | | AAGAAATTGCTCATGGAAACAACAAGCAAGTCCATCATTG |
| | | TCATTGAAGATATCGATTGTTCTATTGATCTCACGGGCAA |
| | | GAGAAAGAAGAAAAATAAAAAGGAAACAACGAATGAGGAG |
| | | GAGGAAGAAGATTCAGACTCGACCACAGAGAAAAAGAACG |
| | | ACAAGAATGAATCTAGCAAACTTACTCTTTCGGGGCTTTT |
| | | GAATTTCATAGATGGAATATGGTCAGCTTGTGGTCAAGAG |
| | | AGAATTATTATATTTACGACTAATCATGTGGACAAACTTG |
| | | ATCCAGCTCTGATACGTAGAGGACGGATGGATATGCACAT |
| | | TGAGATGTCGTATTGCAAATATGAAGCATTCAAAGTGTTG |
| | | GCTAAGAATTACTTGGGAATCGAAACACATCCTCTGTTTC |
| | | AAGAGATTCAAAGTTTATTAGAGGAAGTAGACGTGAGTCC |
| | | TTGTGATGTGGCTGAGAACTTGATGCCCAAAAATGCTTCA |
| | | GGAAACCCTGAGATTTGCTTGGAAAGCTTAGTCAAAGTTT |
| | | TGATAAAGGCCAAGAGAAAGGCCAGCTTGAATTCCCACAA |
| | | GAACAAAAAATCCTCCACAAAACTAATCATAACTTATGGC |
| | | CGTTTCAAAAAGCTGTTTCAATGA |
| SEQ | Solyc08g083120.2 | ATGCAGATCTTCGTGAAAACTTTGACGGGTAAAACCATAA |
| ID | | CCCTCGAGGTTGAATCCAGTGACACTATTGACAATGTCAA |
| NO: | | GGCCAAGATTCAAGACAAGGAAGGAATTCCTCCAGACCAG |
| 240 | | CAAAGGCTGATTTTTGCTGGTAAGCAGCTAGAAGATGGCC |
| | | GCACTCTTGCTGACTATAACATTCAGAAAGAGTCGACTTT |
| | | GCATTTGGTGCTGAGGCTTCGTGGAGGAATTATTGAGCCT |
| | | TCTTTGATGGCTTTGGCTAGGAAGTACAATCAGGAAAAGA |
| | | TGATTTGCCGCAAGTGCTACGCTCGCCTACATCCCCGTGC |
| | | TGTCAACTGCCGCAAGAAGAAGTGTGGGCACAGCAACCAG |
| | | TTGAGGCCTAAGAAGAAGATCAAGTAG |
| SEQ | Solyc05g050200.4 | ATGCCGAAGAACAAGGGTAAGGGAGGTAAGAACAGGAAGA |
| ID | | GAGGAAAGAATGAAGCCGATGATGAGAAAAGAGAGCTTGT |
| NO: | | TTTCAAAGAAGATGGACAGGAATATGCTCAGGTCCTTCGT |
| 241 | | ATGCTTGGTAATGGACGATGTGAAGCTATGTGTATTGATG |
| | | GAACTAAGCGTCTTTGTCACATACGTGGTAAGATGCATAA |
| | | GAAAGTATGGATCGCTGCTGGTGATATCATCCTTGTTGGC |
| | | CTACGTGACTATCAGGATGACAAAGCTGATGTGATCTTGA |
| | | AGTACATGCCTGATGAGGCCAGGTTATTGAAGGCATATGG |
| | | AGAGTTACCAGAGAACACAAGACTTAATGAGGGCATTGCT |
| | | AATGGTCTTGATGAAGAGGATGAGGGTGCTGGTGATGATT |
| | | ACATTGAGTTTGAGGATGAAGACATTGATAAAATCTAA |
| SEQ | Solyc11g005670.2 | ATGCAGATATTTGTTAAGACACTCACCGGAAAGACCATCA |
| ID | | CTCTTGAGGTTGAGAGTTCTGACACCATTGATAATGTCAA |
| NO: | | AGCTAAGATTCAAGACAAGGAAGGCATTCCTCCAGATCAG |
| 242 | | CAGAGACTGATCTTTGCTGGGAAACAGCTTGAAGATGGCC |
| | | GAACACTTGCTGATTACAACATCCAAAAAGAGTCTACCCT |
| | | TCATCTTGTCCTCCGTCTACGTGGTGGGATGCAAATCTTT |
| | | GTTAAAACTCTGACCGGAAAGACTATAACTCTTGAGGTCG |
| | | AGAGCTCAGACACCATCGATAATGTCAAAGCTAAAATTCA |
| | | AGACAAGGAAGGTATTCCCCCAGACCAGCAGAGGCTGATC |
| | | TTTGCTGGGAAACAGCTTGAAGATGGCCGAACACTTGCTG |
| | | ATTACAACATCCAAAAGGAGTCCACCCTTCACCTTGTCCT |
| | | TCGCCTTCGTGGTGGTATGCAGATCTTTGTCAAGACACTT |
| | | ACTGGAAAGACCATCACCCTTGAAGTTGAAAGTTCCGATA |
| | | CTATTGACAATGTGAAGGCCAAAATTCAGGATAAGGAAGG |
| | | GATTCCTCCAGACCAGCAGAGACTGATCTTTGCCGGCAAG |
| | | CAACTTGAGGATGGAAGGACCCTGGCTGACTACAATATTC |
| | | AGAAAGAGTCTACCTTACATCTTGTTCTTCGACTGAGGGG |
| | | TGGCATGCAAATATTTGTCAAGACATTGACAGGGAAGACA |
| | | ATTACTTTGGAGGTTGAGAGTTCAGATACTATCGACAATG |
| | | TTAAAGCAAAGATACAAGACAAGGAGGGTATTCCTCCAGA |
| | | CCAGCAGAGATTGATCTTTGCTGGAAAGCAACTTGAGGAT |
| | | GGAAGGACGTTGGCAGATTACAACATTCAGAAAGAATCAA |
| | | CCCTGCACCTTGTTCTTCGCCTTAGAGGTGGCATGCAAAT |
| | | TTTTGTCAAGACTTTGACTGGGAAGACAATTACATTGGAG |
| | | GTTGAGAGTTCAGATACCATTGACAATGTGAAAGCAAAGA |
| | | TTCAAGACAAGGAGGGTATTCCCCCAGACCAACAGCGTTT |
| | | GATCTTTGCTGGTAAACAACTTGAGGACGGGAGGACTCTT |
| | | GCAGACTACAACATCCAGAAGGAGTCAACTCTCCATCTGG |
| | | TGTTGCGTTTGAGAGGAGGGATGCAGATCTTTGTGAAGAC |
| | | TTTGACTGGGAAGACAATCACATTGGAGGTTGAGAGTTCA |
| | | GATACCATTGACAATGTGAAAGCAAAGATTCAAGACAAGG |
| | | AGGGTATTCCCCCAGACCAACAGCGTTTGATCTTTGCTGG |
| | | TAAACAACTTGAGGATGGGAGGACTCTTGCAGACTACAAC |
| | | ATCCAGAAGGAGTCAACTCTCCATCTGGTGTTGCGTTTGA |
| | | GAGGAGGGATGCAGATCTTTGTGAAGACTCTTACTGGGAA |
| | | GACAATCACATTGGAGGTGGAGAGCTCTGATACTATTGAC |
| | | AATGTGAAAGCAAAGATACAGGACAAGGAAGGGATCCCAC |
| | | CAGATCAACAGAGGCTCATCTTTGCTGGTAAGCAGCTTGA |
| | | GGATGGCCGCACCCTTGCAGACTATAACATCCAGAAAGAG |
| | | TCTACTCTTCATCTTGTCCTGAGGCTCCGTGGCGGTGACT |
| | | TGTGA |
| SEQ | Solyc12g088210.3 | ATGCAGATATTTGTTAAAACCCTCACTGGAAAGACTATCA |
| ID | | CCCTTGAGGTGGAAAGCTCAGACACCATTGACAATGTTAA |
| NO: | | AGCCAAGATCCAGGACAAGGAAGGCATTCCCCCAGACCAG |
| 243 | | CAGAGGCTGATCTTTGCAGGAAAGCAGCTTGAAGACGGTC |
| | | GTACTCTAGCTGATTACAACATTCAGAAAGAGTCAACTCT |
| | | CCACTTGGTGCTCCGTCTTCGTGGTGGCATGCAAATTTTC |
| | | GTGAAAACTTTGACAGGAAAGACCATCACCCTTGAGGTTG |
| | | AAAGCTCTGACACCATTGACAATGTTAAGGCTAAGATCCA |
| | | AGACAAGGAAGGCATTCCCCCAGACCAGCAAAGGTTGATC |
| | | TTTGCGGGCAAGCAGCTTGAGGACGGCAGGACCCTTGCTG |
| | | ATTACAACATCCAGAAGGAATCAACCCTTCACTTGGTGCT |
| | | CCGTCTTCGTGGTGGAATGCAAATTTTTGTGAAGACTTTG |
| | | ACTGGAAAGACCATCACCCTTGAGGTTGAAAGCTCCGACA |
| | | CCATTGACAATGTTAAGGCTAAGATCCAAGACAAGGAAGG |
| | | CATTCCCCCAGACCAGCAAAGGTTGATCTTTGCGGGCAAG |
| | | CAGCTTGAGGACGGCAGGACCCTTGCTGATTACAACATCC |
| | | AGAAGGAGTCAACCCTTCACTTGGTCCTTCGTCTTCGCGG |
| | | TGGGATGCAAATCTTTGTGAAGACCTTGACTGGCAAGACA |
| | | ATCACCCTTGAGGTGGAAAGTTCTGACACCATTGACAATG |
| | | TTAAGGCCAAAATTCAGGACAAAGAAGGTATTCCACCAGA |
| | | CCAGCAGAGGTTGATCTTTGCCGGAAAGCAACTTGAGGAC |
| | | GGCCGTACCCTTGCTGATTACAACATCCAGAAGGAGTCAA |
| | | CCCTTCACTTGGTCCTGCGTCTCCGAGGTGGAATGCAAAT |
| | | CTTCGTGAAGACCTTGACTGGCAAGACTATTACCCTTGAG |
| | | GTGGAAAGCTCCGACACCATTGACAACGTAAAGGCCAAGA |
| | | TTCAGGACAAAGAGGGTATCCCACCAGACCAGCAAAGGTT |
| | | GATTTTTGCTGGCAAGCAGTTGGAAGATGGAAGGACATTA |
| | | GCCGATTACAATATTCAGAAGGAATCAACCCTTCATCTTG |
| | | TGTTGAGGCTCAGGGGAGGTATGCAGATTTTTGTGAAGAC |
| | | CTTGACTGGGAAAACCATAACTTTGGAGGTGGAGAGTTCT |
| | | GATACCATTGACAATGTGAAGGCTAAAATTCAGGACAAGG |
| | | AGGGAATCCCACCGGACCAGCAGAGACTGATCTTTGCAGG |
| | | GAAGCAACTTGAAGATGGAAGGACTCTGGCGGACTACAAC |
| | | ATCCAAAAGGAGTCTACTTTGCACCTTGTCCTCCGTCTCC |
| | | GTGGTGGTTTTTGA |
| SEQ | Solyc12g099000.3 | ATGGAGACTTTCTTATTCACATCTGAATCAGTCAACGAGG |
| ID | | GACATCCCGACAAGCTCTGTGACCAGGTCTCTGATGCAGT |
| NO: | | GCTCGATGCCTGTCTAGCTCAGGACCCTGAAAGCAAAGTT |
| 244 | | GCATGTGAGACTTGTACCAAGACCAACTTAGTTATGGTCT |
| | | TTGGAGAGATCACCACCAAGGCCAATATTGATTATGAGAA |
| | | GATTGTACGTGACACTTGCCGGGAAATTGGATTTGTGTCC |
| | | CCTGATGTTGGTTTGGATGCTGACAATTGCAGAGTCCTTG |
| | | TGAACATTGAGCAGCAGAGCCCTGATATTGCTCAAGGTGT |
| | | TCATGGTCATTTGACTAAGCGACCTGAGGAGATTGGTGCT |
| | | GGTGACCAGGGTCACATGTTTGGCTATGCCACTGACGAGA |
| | | CACCGGAGTTGATGCCCCTTAGCCATGTTCTTGCTACCAA |
| | | ACTTGGAGCTCGCCTTACTGAGGTCCGCAAGAATGGAACT |
| | | TGCTCTTGGCTTAGACCTGATGGTAAAACACAAGTGACTG |
| | | TTGAGTATCACAATGACAATGGTGCTATGGTTCCTCTACG |
| | | TGTTCACACTGTTTTAATCTCCACTCAGCATGATGAGACT |
| | | GTTACCAATGATGAAATTGCTCGTGATCTCAAAGAGCATG |
| | | TCATCAAGCCTGTCATCCCCGAGAAGTATCTTGATGAGAA |
| | | CACCATTTTCCACCTCAACCCTTCAGGCCGTTTTGTCATT |
| | | GGTGGACCTCACGGTGATGCTGGTCTCACTGGCCGTAAGA |
| | | TCATCATTGACACTTACGGAGGTTGGGGTGCTCACGGAGG |
| | | TGGTGCTTTCTCTGGGAAGGATCCTACCAAGGTCGACAGA |
| | | AGTGGAGCCTATATCGTGAGGCAAGCAGCTAAGAGCATTG |
| | | TTGCTAACGGTCTTGCCAGAAGGTGCATTGTTCAGGTTTC |
| | | ATATGCCATCGGTGTGCCTGAGCCATTGTCCGTCTTTGTG |
| | | GACACTTATGGAACTGGGAAGATCCCTGACAAGGAGATTC |
| | | TCAACATTGTGAAGGAGAACTTTGATTTCAGGCCCGGAAT |
| | | GATTTCCATTAACCTCGATCTACTGAGGGGTGGCAATGGT |
| | | CGATTCTTGAAGACTGCTGCCTATGGGCATTTTGGTAGAG |
| | | ATGATCCTGACTTCACATGGGAAGTAGTTAAGCCTCTCAA |
| | | GTGGGACAAGCCTGAAGCTTAA |
| SEQ | Solyc01g096000.2 | ATGAATGATCCTTACAGATCTAAACCCAGTAGCGGGCGAA |
| ID | | CAAATTTTGCTTCATGTATTTGTGCCACTGTCTTCTTGAT |
| NO: | | CTTTCTTATTGCAGGCATTGTTGTCGCTTACTTCTTCCTC |
| 245 | | TTCAAACCCAAAACTCCCAAAATCGCCGTTGAGGCAGTTC |
| | | GATTCCCTACTTTCTCTGTTACCAACGGTACTGTTAACTT |
| | | CACCTTCTTCCAGTACGTCTCCGTCACAAACCCAAATCGG |
| | | GATGAGTTTACTCACTATGACAGTTCGCTTCAGCTCAGTT |
| | | ACTCAGGGGAGCCGGTGGGTCTTGTTTTTATTCCCGCCGG |
| | | GAAGATCGACGGTGGTAGGACCCAGCATATGTCGGCGAAA |
| | | TTTGATGTTCAGTCTTACCCACTTCCAGCCAAGCTGAAAG |
| | | CTGATGTCTCCGGCGGGATGATTCCGACGACGGCGGTGAA |
| | | CGTCTTTGATGAGGGACCCACAATGGAGGTGGAAACGAGG |
| | | ATGAAGTTGGTGGGAAGAGTTAGGGTATTGAAAGTGTTTA |
| | | CACATAGGGTGGACAGTGGAGTGAAATGTGGAGTAATGAT |
| | | TCAAGTCAGTAGTGGCTCTGTTTTAGGCGTCCGTTGCTGA |
| SEQ | Solyc10g006480.2 | ATGCAGATATTTGTTAAAACCCTCACTGGAAAGACTATCA |
| ID | | CCCTTGAGGTGGAAAGCTCAGACACCATTGACAATGTTAA |
| NO: | | AGCCAAGATCCAGGACAAGGAAGGCATTCCCCCAGACCAG |
| 246 | | CAGAGGCTGATCTTTGCAGGAAAGCAGCTTGAAGACGGTC |
| | | GTACTCTAGCTGATTACAACATTCAGAAAGAGTCAACTCT |
| | | CCACTTGGTGCTCCGTCTTCGTGGTGGCATGCAAATTTTC |
| | | GTGAAAACTTTGACAGGAAAGACCATCACCCTTGAGGTTG |
| | | AAAGCTCTGACACCATTGACAATGTTAAGGCTAAGATCCA |
| | | AGACAAGGAAGGCATTCCCCCAGACCAGCAAAGGTTGATC |
| | | TTTGCGGGCAAGCAGCTTGAGGACGGCAGGACCCTTGCTG |
| | | ATTACAACATCCAGAAGGAATCAACCCTTCACTTGGTGCT |
| | | CCGTCTTCGTGGTGGAATGCAAATTTTTGTGAAGACTTTG |
| | | ACTGGAAAGACCATCACCCTTGAGGTTGAAAGCTCCGACA |
| | | CCATTGACAATGTTAAGGCTAAGATCCAAGACAAGGAAGG |
| | | CATTCCCCCAGACCAGCAAAGGTTGATCTTTGCGGGCAAG |
| | | CAGCTTGAGGACGGCAGGACCCTTGCTGATTACAACATCC |
| | | AGAAGGAGTCAACCCTTCACTTGGTCCTTCGTCTTCGCGG |
| | | TGGGATGCAAATCTTTGTGAAGACCTTGACTGGCAAGACA |
| | | ATCACCCTTGAGGTGGAAAGTTCTGACACCATTGACAATG |
| | | TTAAGGCCAAAATTCAGGACAAAGAAGGTATTCCACCAGA |
| | | CCAGCAGAGGTTGATCTTTGCCGGAAAGCAACTTGAGGAC |
| | | GGCCGTACCCTTGCTGATTACAACATCCAGAAGGAGTCAA |
| | | CCCTTCACTTGGTCCTGCGTCTCCGAGGTGGAATGCAAAT |
| | | CTTCGTGAAGACCTTGACTGGCAAGACTATTACCCTTGAG |
| | | GTGGAAAGCTCCGACACCATTGACAACGTAAAGGCCAAGA |
| | | TTCAGGACAAAGAGGGTATCCCACCAGACCAGCAAAGGTT |
| | | GATTTTTGCTGGCAAGCAGTTGGAAGATGGAAGGACATTA |
| | | GCCGATTACAATATTCAGAAGGAATCAACCCTTCATCTTG |
| | | TGTTGAGGCTCAGGGGAGGTATGCAGATTTTTGTGAAGAC |
| | | CTTGACTGGGAAAACCATAACTTTGGAGGTGGAGAGTTCT |
| | | GATACCATTGACAATGTGAAGGCTAAAATTCAGGACAAGG |
| | | AGGGAATCCCACCGGACCAGCAGAGACTGATCTTTGCAGG |
| | | GAAGCAACTTGAAGATGGAAGGACTCTGGCGGACTACAAC |
| | | ATCCAAAAGGAGTCTACTTTGCACCTTGTCCTCCGTCTCC |
| | | GTGGTGGTTTTTGA |
| SEQ | Solyc01g095995.2 | ATGAATGATCCTTACAGATCTAAACCCAGTAGCGGGCGAA |
| ID | | CAAATTTTGCTTCATGTATTTGTGCCACTGTCTTCTTGAT |
| NO: | | CTTTCTTATTGCAGGCATTGTTGTCGCTTACTTCTTCCTC |
| 247 | | TTCAAACCCAAAACTCCCAAAATCGCCGTTGAGGCAGTTC |
| | | GATTCCCTACTTTCTCTGTTACCAACGGTACTGTTAACTT |
| | | CACCTTCTTCCAGTACGTCTCCGTCACAAACCCAAATCGG |
| | | GATGAGTTTACTCACTATGACAGTTCGCTTCAGCTCAGTT |
| | | ACTCAGGGGAGCCGGTGGGTCTTGTTTTTATTCCCGCCGG |
| | | GAAGATCGACGGTGGTAGGACCCAGCATATGTCGGCGAAA |
| | | TTTGATGTTCAGTCTTACCCACTTCCAGCCAAGCTGAAAG |
| | | CTGATGTCTCCGGCGGGATGATTCCGACGACGGCGGTGAA |
| | | CGTCTTTGATGAGGGACCCACAATGGAGGTGGAAACGAGG |
| | | ATGAAGTTGGTGGGAAGAGTTAGGGTATTGAAAGTGTTTA |
| | | CACATAGGGTGGACAGTGGAGTGAAATGTGGAGTAATGAT |
| | | TCAAGTCAGTAGTGGCTCTGTTTTAGGCGTCCGTTGCTGA |

The details discussed above for overexpressing, underexpressing, silencing and inactivating genes in a plant cell or a plant are also applicable to the methods of identifying a gene that induces RKN resistance in a plant cell or a plant when overexpressed, silenced, underexpressed, or inactivated and such embodiments are within the purview of the invention.

Methods of the present disclosure may be used for the treatment of any suitable plant type, including, but not limited to, row crops and vegetables. In some embodiments, inoculant compositions of the present disclosure are formulated for the treatment of one or more plants selected from Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalo grass, Centipede grass, Fescue, or Zoysia), millet, oats, ornamental grasses, rice, rye, sorghum, sugar cane, triticale, wheat), Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape). In some embodiments, inoculant compositions of the present disclosure are formulated for the treatment of one or more fungicide-, herbicide-, insecticide- and/or nematicide-resistant plants (e.g., one or more plants resistant to acetolactate synthase inhibitors.

Exemplary embodiments of such methods are described in Examples 1 to 6 below. A skilled artisan can implement such methods to inhibit RKN infections in plants and such embodiments are within the purview of the invention.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Construction of Gall-Specific Yeast Two-Hybrid Prey Library

Seeds of the RKN-susceptible tomato cultivar Heinz 1706 were germinated in magenta boxes on Murashige and Skoog (MS) medium, and seven days after emergence, tomato seedlings were transplanted into a cone-tainer filled with sterile soil/sand mixture. Five days after transplantation, each seedling was inoculated with about 100 second stage juveniles (J2) of Meloidogyne incognita. Plants were growing in a growth chamber at 26° C. and 16h light/8 h dark photoperiod. Galls were dissected from the infected roots at 4, 7, and 11 days post infection (dpi) in three biological samples. Total RNA was extracted from the gall samples using Direct-zol RNA miniprep kit (Zymo Research, Irvine, CA), following manufacturer's instructions. About 200 μg total RNA from each time point were combined and used to isolate mRNA using Purification of poly(A) RNA User manual NucleoTrap® mRNA (Macherey-Nagel Kit, Düren, Germany). The prey library was constructed using Make Your Own “Mate & Plate™” Library System (Clontech (Takara Bio Inc., Kusatsu, Shiga, Japan)). In brief, 1.8 μg mRNA were used to synthesize first-strand cDNA with an oligo-dT. Synthesized cDNAs were amplified using Long-Distance PCR with 18 cycles. Double-stranded cDNA (ds cDNA) were then purified with CHROMA SPIN+TE-400 Columns (Clontech). Then, yeast competent cells (Y187 strain) were transformed with 5.1 μg ds cDNA and 3 μg of the prey vector pGADT7-Rec (Clontech). The transformed cells were plated on SD/-leucine plates and incubated at 30° C. for five days. Then, transformed yeast colonies were collected using liquid freezing medium. Cell density of the prey library was estimated using hemocytometer and found to be 3.4×107 cells/ml, reflecting the high quality of the prey library.

### Example 2—Cloning 11 Meloidogyne Incognita Effectors in Bait Vector

The coding sequences of 11 Meloidogyne incognita effectors were cloned in frame with the GAL4 DNA binding domain (BD) of the bait vector pGBKT7 (Clontech) to generate the bait vectors. These nematode effectors include Effector 1 (Minc13292), Effector 2 (Minc18861), Effector 3 (Minc00801), Effector 4 (Minc02097), Effector 5 (Minc18033), Effector 6 (Minc01696), Effector 7 (Minc00344), Effector 8 (Minc00469), Effector 9 (Minc15401), Effector 11 (Minc12639), and Effector 12 (Minc03328) (Rutter et al., 2014). Effectors 1, 5, 6, 7, 8, 9, and 12 are localized in the nematode subventral gland cells, which are most active during the early stage of infection. Effectors 2, 4, and 11 are localized in the nematode dorsal glad cell, which is active during the sedentary stage of nematode infection. Effector 3 is localized in rectal gland of the adult female nematodes (Rutter et al., 2014). The 11 bait vectors were confirmed by sequencing and then transformed into competent yeast strain Y2HGold, and tested for autoactivation and toxicity as described in Matchmaker Gold Yeast Two-Hybrid System User Manual (Clontech). The results confirmed that all the 11 baits are not toxic and do not autonomously activate the reporter genes in Y2HGold yeast strain. Therefore, we used these11 effectors for systemic yeast two-hybrid (Y2H) screens using the gall-specific prey libraries.

### Example 3—Yeast Two-Hybrid Screens and Identification of Tomato Interacting Proteins

We performed Y2H screens for each of these 11 effectors separately, using very stringent selection criteria, which produced high-quality data with very low false positives. Our Y2H screen pipeline included primary screens using the stringent quadruple dropout SD/-Ade/-His/-Leu/-Trp medium, secondary phenotyping screens using the quadruple dropout SD/-Ade/-His/-Leu/-Trp/X-alpha-Gal medium to visualize the activity of alpha-galactosidase encoded by the MEL1 reporter gene (positive yeast colonies expressing the Mell reporter gene turn blue in the presence of the chromogenic substrate X-alpha-Gal) (FIG. 1), and finally growth rate in the SD/-Ade/-His/-Leu/-Trp liquid medium. Additional confirmation including yeast co-transformation assays of bait and prey plasmids in the Y2HGold yeast strain as previously described (Hewezi et al., 2008; 2010; 2015; 2016). A total of 339 positive yeast colonies were identified including 7, 35, 24, 28, 32, 26,30,63, 33, 47, and 14 colonies for effector 1 to 12, respectively. The prey plasmids were isolated form yeast cells, transformed into E. coli, and finally purified and sequenced.

### Example 4—Meloidogyne Incognita—Tomato Interactome

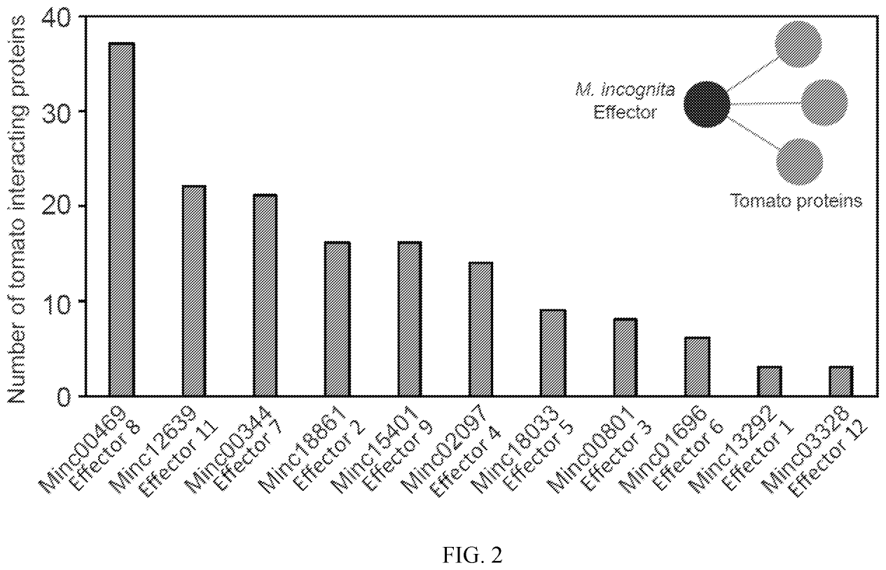

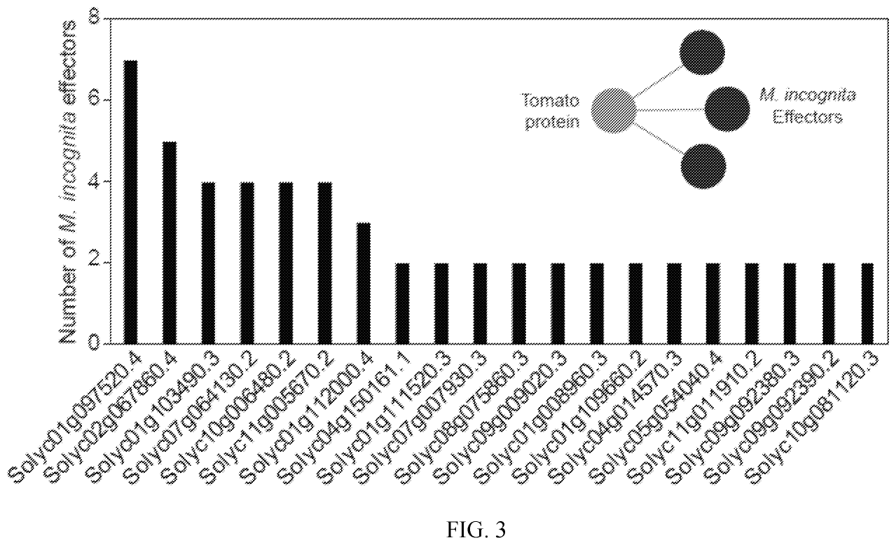

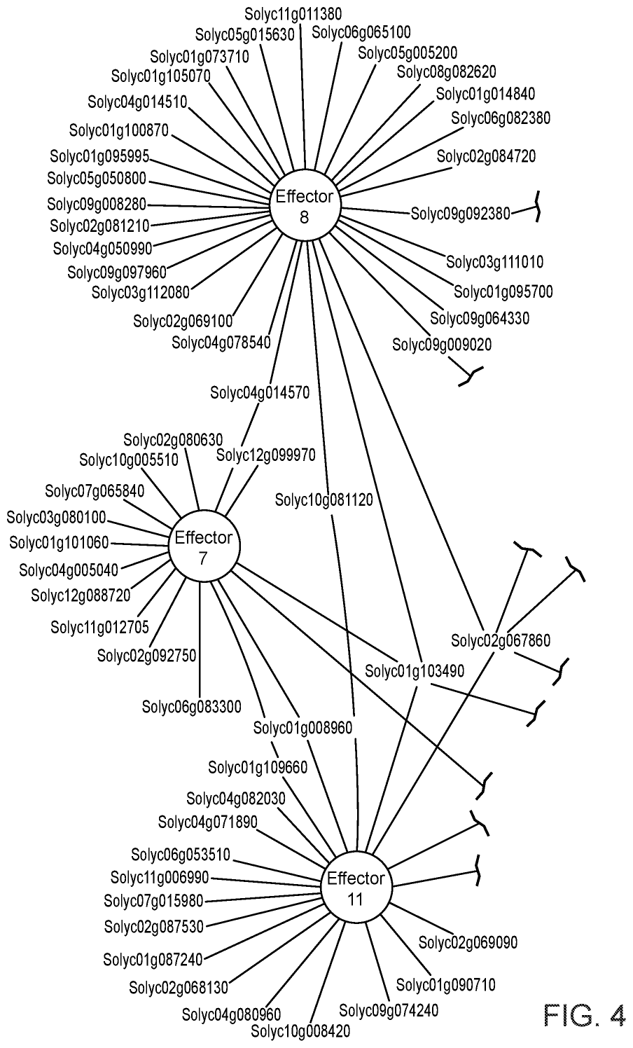

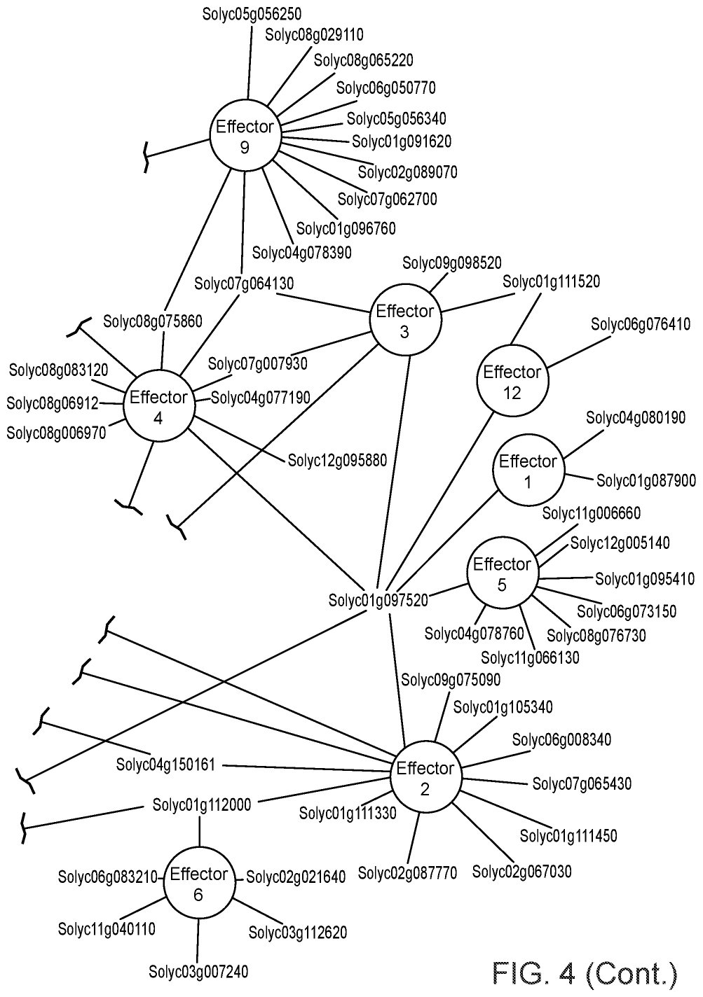

Sequence analysis of the 339 prey plasmids revealed that the 11 nematode effectors interact with 118 unique tomato proteins (Table 1). The number of tomato proteins interacting with individual effector varies between 3 (for effectors 1 and 12) and 37 (for effector 8) (FIG. 2, and Tables 4A-4C), Interestingly, we found 20 tomato proteins targeted by at least two nematode effectors (FIG. 3). For example, the tomato proteins Solyc01g097520.4 and Solyc02g067860.4 were targeted by 7 and 5 different nematode effectors, respectively (FIG. 3). Solyc01g103490.3, Solyc07g064130.2, Solyc10g006480.2, and Solyc11g005670.2 each is targeted by 4 different nematode effectors (FIG. 3). Solyc01g112000.4 was targeted by three different nematode effectors. Thirteen proteins (Solyc04g150161.1, Solyc01g111520.3, Solyc07g007930.3, Solyc08g075860.3, Solyc09g009020.3, Solyc01g008960.3, Solyc01g109660.2, Solyc04g014570.3, Solyc05g054040.4, Solyc11g011910.2, Solyc09g092380.3, Solyc09g092390.2, and Solyc10g081120.3) each is targeted by two different nematode effectors. Each of the remaining 98 tomato proteins is targeted by one nematode effector. The network of 118 tomato proteins interacting with 11 M. incognita effectors is provided in FIG. 4.

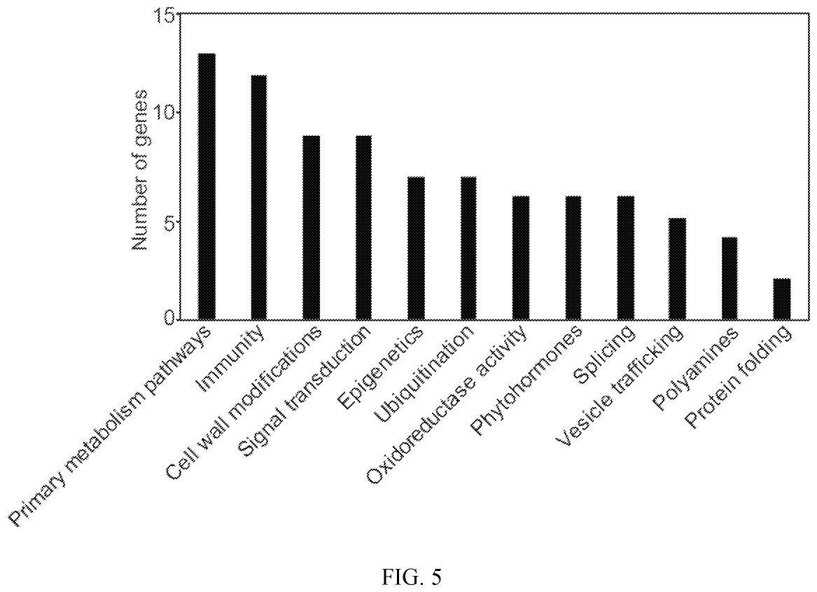

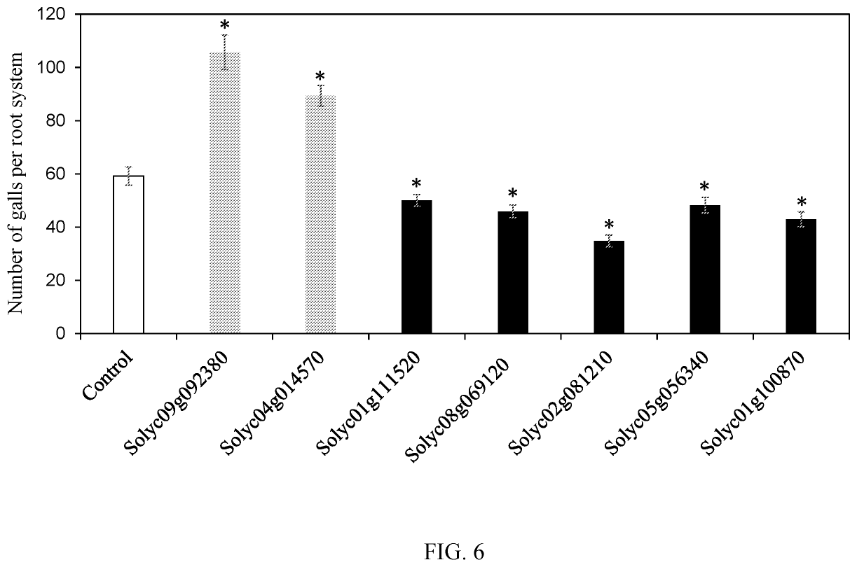

Gene ontology (GO) classification and pathway analysis of 118 tomato proteins targeted by the 11 nematode effectors revealed their involvement in various biological processes and molecular functions required for nematode fitness. For instance, several tomato targets are involved in immunity, protein folding and ubiquitination, vesicle trafficking, primary metabolism pathways, oxidoreductase activity, polyamine biosynthesis, epigenetics and gene silencing, splicing, signal transduction, and cell wall modifications (FIG. 5). These functions have been shown to be involved in plant-nematode interactions (Hewezi and Baum 2013; Goverse and Smant 2014; Hewezi, 2015; Fosu-Nyarkoand Jones, 2016; Siddique and Grundler 2018; Hu and Hewezi, 2018; Gheysen and Mitchum 2019; Mejias et al., 2019; Vieira and Gleason 2019; Hewezi 2020).

TABLE 4A
Accession numbers of tomato genes encoding proteins interacting
with each of the 11 tested Meloidogyne incognita effectors.
Effector Number

| | | | | |
|---|---|---|---|---|
| Effector #1 | Effector #2 | Effector #3 | Effector #4 | Effector #5 |

Effector ID

| | | | | |
|---|---|---|---|---|
| Minc13292 | Minc18861 | Minc00801 | Minc02097 | Minc18033 |

Number of tomato interacting proteins

| 3 | 16 | 8 | 14 | 9 |
|---|---|---|---|---|
| Solyc01g097520 | Solyc02g067030 | Solyc07g007930 | Solyc12g088210 | Solyc01g095410 |
| Solyc04g080190 | Solyc01g097520 | Solyc10g006480 | Solyc04g077190 | Solyc05g050200 |
| Solyc01g087900 | Solyc02g087770 | Solyc07g064130 | Solyc09g009020 | Solyc12g005140 |
| | Solyc06g008340 | Solyc11g005670 | Solyc08g083120 | Solyc04g078760 |
| | Solyc09g075090 | Solyc01g097520 | Solyc08g069120 | Solyc01g097520 |
| | Solyc01g112000 | Solyc01g111520 | Solyc07g064130 | Solyc06g073150 |
| | Solyc01g111330 | Solyc02g067860 | Solyc10g006480 | Solyc08g076730 |
| | Solyc01g103490 | Solyc09g098520 | Solyc11g005670 | Solyc11g006660 |
| | Solyc07g065430 | | Solyc07g007930 | Solyc11g066130 |
| | Solyc01g111450 | | Solyc02g067860 | |
| | Solyc01g105340 | | Solyc08g006970 | |
| | Solyc04g150161 | | Solyc08g075860 | |
| | Solyc11g005670 | | Solyc12g095880 | |
| | Solyc10g006480 | | Solyc01g097520 | |
| | Solyc07g064130 | | | |
| | Solyc02g067860 | | | |

TABLE 4B
Accession numbers of tomato genes encoding proteins interacting
with each of the 11 tested Meloidogyne incognita effectors.
Effector Number
Effector #8
Effector ID
Minc00469
Number of tomato interacting proteins
37

| | | | |
|---|---|---|---|
| Solyc01g096000 | Solyc09g064330 | Solyc04g014570 | Solyc01g073710 |
| Solyc04g050990 | Solyc01g100870 | Solyc05g054040 | Solyc08g082620 |
| Solyc02g084720 | Solyc01g095700 | Solyc11g011910 | Solyc06g065100 |
| Solyc05g050800 | Solyc01g096580 | Solyc11g011380 | Solyc01g014840 |
| Solyc02g081210 | Solyc01g096590 | Solyc03g112080 | Solyc02g067860 |
| Solyc09g097960 | Solyc05g005200 | Solyc04g014510 | Solyc01g095995 |
| Solyc01g105070 | Solyc05g015630 | Solyc09g009020 | Solyc10g081120 |
| Solyc02g069100 | Solyc05g014470 | Solyc01g103490 | Solyc04g078540 |
| Solyc09g092390 | Solyc03g111010 | Solyc06g082380 | Solyc09g008280 |
| Solyc09g092380 | | | |

TABLE 4C
Accession numbers of tomato genes encoding proteins interacting
with each of the 11 tested Meloidogyne incognita effectors.
Effector Number

| | | | | |
|---|---|---|---|---|
| Effector #6 | Effector #7 | Effector #9 | Effector #11 | Effector #12 |

Effector ID

| | | | | |
|---|---|---|---|---|
| Minc01696 | Minc00344 | Minc15401 | Minc12639 | Minc03328 |

Number of tomato interacting proteins

| 6 | 21 | 16 | 22 | 3 |
|---|---|---|---|---|
| Solyc03g007240 | Solyc01g103490 | Solyc01g091620 | Solyc06g053510 | Solyc01g111520 |
| Solyc03g112620 | Solyc06g072120 | Solyc07g062700 | Solyc04g071890 | Solyc06g076410 |
| Solyc06g083210 | Solyc06g083300 | Solyc02g089070 | Solyc02g068130 | Solyc01g097520 |
| Solyc11g040110 | Solyc12g099000 | Solyc07g064130 | Solyc02g067860 | |
| Solyc02g021640 | Solyc01g101060 | Solyc10g006480 | Solyc05g007950 | |
| Solyc01g112000 | Solyc03g080100 | Solyc11g005670 | Solyc10g008420 | |
| | Solyc11g012705 | Solyc08g029110 | Solyc07g015980 | |
| | Solyc04g014570 | Solyc05g056340 | Solyc04g082030 | |
| | Solyc05g054040 | Solyc01g096760 | Solyc09g074240 | |
| | Solyc11g011910 | Solyc04g078390 | Solyc02g087530 | |
| | Solyc01g008960 | Solyc05g056250 | Solyc01g109660 | |
| | Solyc12g099970 | Solyc09g092390 | Solyc01g008960 | |
| | Solyc12g088720 | Solyc09g092380 | Solyc01g103490 | |
| | Solyc02g080630 | Solyc06g050770 | Solyc01g112000 | |
| | Solyc10g005510 | Solyc08g075860 | Solyc01g087240 | |
| | Solyc07g065840 | Solyc08g065220 | Solyc01g090710 | |
| | Solyc01g109660 | | Solyc01g097520 | |
| | Solyc04g005040 | | Solyc04g080960 | |
| | Solyc06g082670 | | Solyc02g069090 | |
| | Solyc02g092750 | | Solyc11g006990 | |
| | Solyc04g150161 | | Solyc10g081120 | |
| | | | Solyc02g087540 | |

### Example 5—RNA-Seq Analysis of Tomato Galls and Adjacent Roots Tissues Upon Infection by Meloidogyne Incognita

To further investigate the transcriptional activity of the 118 tomato genes coding for proteins interacting with the 11 nematode effectors, we generated RNA-seq libraries from galls and adjacent roots tissues from tomato plants at 4- and 11-days post infection (dpi) with M. incognita. Normalized read counts revealed that 116 of the 118 genes are transcriptionally active in the galls induced by M. incognita in tomato roots, supporting the abundance of the encoded proteins for functional interactions with the examined nematode effectors (Table 5). Also, we discovered that 71 out of the 118 genes are differentially expressed in the galls or in the neighboring roots tissues, indicating changes in the transcriptional activity of these genes in response to nematode infection. The remaining 47 genes seem to be altered at post-translational level after nematode infection.

TABLE 5
Expression levels of 118 tomato genes encoding proteins interacting with the 11 tested Meloidogyne incognita effectors
in the galls and adjacent roots tissues at 4- and 11- day post infection with Meloidogyne incognita. Gene expression
values with an asterisk before the value indicate statistically significant differences (false discovery rate (FDR)
<5%) in the galls or adjacent roots tissues as compared with non-infected control root tissues.

| | Galls at 4 dpi | | | Adjacent root tissues at 4 dpi | | | Galls at 11 dpi | | | Adjacent root tissues at 11 dpi | | |
|---|---|---|---|---|---|---|---|---|---|---|---|---|
| | TPM | | | TPM | | | TPM | | | TPM | | |
| Gene Id | avg. | log2FC | FDR | avg. | log2FC | FDR | avg. | log2FC | FDR | avg. | log2FC | FDR |
| Solyc05g005200.3 | *47.29 | *1.06 | *0.03 | *49.22 | *1.12 | *0.02 | 40.64 | 0.79 | 0.14 | *54.51 | *1.22 | *0.02 |
| Solyc02g067030.4 | 10.52 | −0.26 | 0.23 | *8.43 | *−0.68 | *0.00 | 11.32 | −0.14 | 0.65 | *9.70 | *−0.45 | *0.03 |
| Solyc09g097960.3 | 232.37 | 0.22 | 0.76 | *371.12 | *0.96 | *0.03 | 240.43 | 0.05 | 0.99 | *474.40 | *1.04 | *0.03 |
| Solyc06g076410.4 | 19.16 | −0.26 | 0.35 | *13.53 | *−0.85 | *0.00 | 18.65 | −0.37 | 0.13 | *17.03 | *−0.51 | *0.04 |
| Solyc02g087540.3 | 6.22 | 0.19 | 0.84 | *11.82 | *1.13 | *0.04 | 5.58 | −0.07 | 0.99 | 11.09 | 1.14 | 0.07 |
| Solyc11g005670.2 | 254.09 | 0.34 | 0.54 | *371.78 | *0.97 | *0.01 | 214.10 | −0.16 | 0.91 | 422.72 | 0.83 | 0.08 |
| Solyc04g071890.3 | *516.04 | *2.01 | *0.00 | 236.90 | 0.95 | 0.10 | 230.43 | 0.72 | 0.29 | 298.49 | 1.08 | 0.11 |
| Solyc05g014470.3 | 289.25 | −0.47 | 0.11 | *181.80 | *−1.06 | *0.00 | 441.56 | −0.05 | 0.98 | 317.79 | −0.52 | 0.12 |
| Solyc10g006480.2 | 118.39 | −0.53 | 0.06 | *80.04 | *−1.06 | *0.00 | 163.93 | −0.23 | 0.57 | 136.66 | −0.46 | 0.18 |
| Solyc11g011380.2 | *84.80 | *−0.74 | *0.01 | *76.76 | *−0.82 | *0.00 | 127.68 | −0.25 | 0.56 | 111.15 | −0.45 | 0.23 |
| Solyc01g087240.3 | 11.34 | −0.49 | 0.27 | *10.09 | *−0.93 | *0.01 | 10.89 | −0.72 | 0.06 | 11.65 | −0.58 | 0.23 |
| Solyc02g021640.3 | *12.40 | *−0.96 | *0.00 | *14.06 | *−0.99 | *0.00 | 22.99 | 0.05 | 0.98 | 16.14 | −0.44 | 0.24 |
| Solyc09g009020.3 | *78.24 | *−0.79 | *0.00 | *66.65 | *−0.97 | *0.00 | 126.40 | −0.31 | 0.36 | 118.70 | −0.39 | 0.27 |
| Solyc12g095880.2 | 12.45 | −0.40 | 0.14 | *11.71 | *−0.53 | *0.03 | 12.34 | −0.43 | 0.10 | 13.25 | −0.34 | 0.33 |
| Solyc03g080100.4 | 21.11 | −0.07 | 0.94 | *12.23 | *−1.22 | *0.02 | 17.06 | −0.67 | 0.31 | 17.07 | −0.73 | 0.33 |
| Solyc12g099000.3 | *279.51 | *0.84 | *0.01 | 216.14 | 0.54 | 0.12 | 257.63 | 0.61 | 0.09 | 229.64 | 0.45 | 0.34 |
| Solyc07g065840.2 | *386.31 | *−0.42 | *0.03 | *314.65 | *−0.63 | *0.00 | 451.72 | −0.33 | 0.12 | 469.38 | −0.26 | 0.35 |
| Solyc12g099970.2 | 35.23 | −0.22 | 0.65 | *28.29 | *−1.07 | *0.00 | 28.43 | −0.36 | 0.41 | 27.46 | −0.41 | 0.39 |
| Solyc01g105070.3 | *250.39 | *1.55 | *0.02 | 108.87 | 0.41 | 0.51 | 191.64 | 1.17 | 0.10 | 152.94 | 0.82 | 0.41 |
| Solyc05g050800.3 | *30.59 | *−1.07 | *0.01 | *27.52 | *−1.15 | *0.01 | *36.88 | *−0.89 | *0.05 | 47.66 | −0.50 | 0.47 |
| Solyc01g095700.3 | 42.60 | −0.43 | 0.51 | *28.33 | *−1.02 | *0.03 | 46.89 | −0.19 | 0.90 | 38.70 | −0.46 | 0.55 |
| Solyc02g069090.3 | 29.80 | −0.32 | 0.28 | *21.41 | *−0.82 | *0.00 | 35.49 | −0.20 | 0.60 | 34.65 | −0.22 | 0.61 |
| Solyc07g065430.2 | 3.65 | 0.61 | 0.28 | *4.98 | *1.00 | *0.04 | 3.09 | 0.51 | 0.41 | 3.55 | 0.42 | 0.61 |
| Solyc04g014570.3 | 48.91 | −0.05 | 0.92 | *32.55 | *−0.63 | *0.02 | 72.37 | 0.35 | 0.26 | 48.40 | −0.22 | 0.63 |
| Solyc01g105340.4 | 13.40 | 0.12 | 0.74 | *10.68 | *−0.51 | *0.02 | 11.10 | −0.18 | 0.61 | 11.49 | −0.18 | 0.65 |
| Solyc04g078540.4 | 88.26 | 0.00 | 1.00 | *52.43 | *−0.72 | *0.04 | 90.54 | 0.03 | 0.99 | 74.16 | −0.26 | 0.67 |
| Solyc11g011910.2 | 22.08 | −0.30 | 0.23 | *19.35 | *−0.51 | *0.02 | 27.30 | −0.02 | 0.99 | 24.86 | −0.16 | 0.68 |
| Solyc05g015630.3 | 16.79 | 0.01 | 0.98 | *12.10 | *−0.58 | *0.00 | 17.88 | 0.18 | 0.44 | 15.23 | −0.13 | 0.69 |
| Solyc01g109660.2 | 790.50 | −0.07 | 0.86 | *572.36 | *−0.47 | *0.03 | 690.09 | −0.17 | 0.64 | 688.02 | −0.15 | 0.70 |
| Solyc09g075090.1 | 26.16 | 0.03 | 0.93 | *20.09 | *−0.45 | *0.05 | 24.93 | −0.08 | 0.93 | 24.14 | −0.15 | 0.71 |
| Solyc06g083210.3 | 12.43 | −0.30 | 0.28 | *11.69 | *−0.46 | *0.05 | 11.96 | −0.33 | 0.22 | 13.71 | −0.14 | 0.73 |
| Solyc06g072120.3 | 108.35 | 0.01 | 0.98 | *64.01 | *−0.68 | *0.01 | 141.45 | 0.31 | 0.33 | 104.62 | −0.12 | 0.79 |
| Solyc03g111010.4 | 30.69 | −0.02 | 0.97 | *20.55 | *−0.67 | *0.01 | 29.66 | −0.25 | 0.54 | 32.72 | −0.12 | 0.80 |
| Solyc11g006660.3 | 64.01 | −0.01 | 0.98 | *39.20 | *−0.72 | *0.01 | 87.34 | 0.35 | 0.34 | 63.48 | −0.13 | 0.80 |
| Solyc01g111520.3 | *45.06 | *0.66 | *0.02 | 30.99 | 0.16 | 0.57 | 42.13 | 0.41 | 0.22 | 34.70 | 0.12 | 0.81 |
| Solyc01g096580.3 | 71.72 | 0.07 | 0.89 | *41.23 | *−0.59 | *0.04 | 85.68 | 0.17 | 0.75 | 67.26 | −0.11 | 0.82 |
| Solyc12g088720.2 | 133.12 | 0.12 | 0.80 | *72.68 | *−0.72 | *0.01 | 152.73 | 0.23 | 0.58 | 119.33 | −0.11 | 0.82 |
| Solyc01g111450.3 | 32.73 | −0.25 | 0.47 | *24.67 | *−0.72 | *0.01 | 52.64 | 0.37 | 0.23 | 38.86 | −0.09 | 0.84 |
| Solyc09g092390.2 | 134.57 | −0.06 | 0.89 | *84.98 | *−0.69 | *0.01 | 159.70 | 0.00 | 1.00 | 150.27 | −0.08 | 0.85 |
| Solyc06g082670.3 | 55.75 | 0.05 | 0.90 | *37.05 | *−0.60 | *0.01 | 69.30 | 0.28 | 0.38 | 56.44 | −0.07 | 0.87 |
| Solyc06g050770.3 | 17.24 | −0.24 | 0.43 | *15.00 | *−0.51 | *0.03 | 21.36 | 0.06 | 0.97 | 19.64 | −0.06 | 0.87 |
| Solyc07g007930.3 | *63.91 | *1.31 | *0.04 | 18.23 | −0.79 | 0.20 | 26.76 | 0.14 | 0.97 | 22.78 | −0.14 | 0.90 |
| Solyc02g080630.4 | 55.18 | −0.08 | 0.87 | *44.51 | *−0.69 | *0.01 | 76.56 | 0.40 | 0.22 | 60.79 | 0.04 | 0.94 |
| Solyc01g096590.3 | 65.35 | 0.12 | 0.73 | *38.22 | *−0.52 | *0.02 | 82.39 | 0.35 | 0.16 | 63.18 | −0.01 | 0.99 |
| Solyc01g095410.3 | 60.89 | −0.11 | 0.82 | *43.51 | *−0.61 | *0.03 | 65.63 | 0.00 | 1.00 | 65.45 | 0.00 | 1.00 |
| Solyc09g064330.2 | 3.75 | 0.22 | 0.77 | *5.97 | *1.05 | *0.02 | 3.77 | 0.26 | 0.77 | 6.40 | 0.98 | NA |
| Solyc10g008420.3 | 14.36 | −0.41 | 0.19 | *12.83 | *−0.79 | *0.00 | *12.40 | *−0.62 | *0.02 | *11.81 | *−0.78 | *0.01 |
| Solyc08g076730.4 | 20.56 | −0.35 | 0.61 | *10.87 | *−1.75 | *0.00 | *14.45 | *−0.96 | *0.04 | *12.54 | *−1.25 | *0.01 |
| Solyc04g014510.3 | *4.49 | *−3.90 | *0.00 | *19.08 | *−1.38 | *0.00 | *11.06 | *−2.14 | *0.00 | *23.44 | *−0.91 | *0.02 |
| Solyc06g065100.3 | 21.57 | −0.73 | 0.07 | *16.71 | *−1.11 | *0.00 | *16.69 | *−1.06 | *0.00 | *18.52 | *−0.92 | *0.03 |
| Solyc04g082030.1 | *409.86 | *0.87 | *0.01 | *345.21 | *0.73 | *0.05 | *587.51 | *1.25 | *0.00 | *433.05 | *0.86 | *0.03 |
| Solyc01g097520.4 | *92.35 | *2.18 | *0.00 | *57.31 | *1.53 | *0.01 | *57.00 | *1.31 | *0.02 | *60.65 | *1.33 | *0.04 |
| Solyc08g006970.4 | *5.74 | *−0.66 | *0.00 | 8.46 | −0.12 | 0.54 | *4.30 | *−0.96 | *0.00 | *6.03 | *−0.47 | *0.05 |
| Solyc04g080960.4 | *189.13 | *−1.17 | *0.00 | *165.84 | *−1.29 | *0.00 | *293.89 | *−0.64 | *0.02 | 317.61 | −0.53 | 0.13 |
| Solyc01g101060.4 | *502.29 | *−0.77 | *0.00 | *481.30 | *−0.75 | *0.00 | *475.94 | *−0.99 | *0.00 | 698.63 | −0.42 | 0.22 |
| Solyc02g081210.3 | 9.35 | 0.49 | 0.09 | 10.22 | 0.47 | 0.09 | *13.41 | *1.18 | *0.00 | 8.76 | 0.40 | 0.26 |
| Solyc10g081120.3 | 17.31 | 0.17 | 0.72 | 11.38 | −0.49 | 0.11 | *10.53 | *−0.84 | *0.00 | 13.85 | −0.44 | 0.27 |
| Solyc02g087770.3 | 26.91 | 0.25 | 0.56 | 16.97 | −0.49 | 0.11 | *40.82 | *0.80 | *0.00 | 32.18 | 0.42 | 0.29 |
| Solyc04g050990.3 | *5.11 | *−1.36 | *0.00 | *8.16 | *−0.93 | *0.05 | *4.57 | *−1.37 | *0.00 | 6.76 | −0.59 | 0.37 |
| Solyc11g066130.1 | 217.09 | 0.40 | 0.14 | *96.86 | *−0.75 | *0.00 | *446.01 | *1.32 | *0.00 | 216.03 | 0.30 | 0.42 |
| Solyc04g077190.4 | 25.88 | 0.28 | 0.67 | 15.72 | −0.54 | 0.20 | *15.56 | *−0.92 | *0.03 | 21.36 | −0.44 | 0.51 |
| Solyc09g008280.2 | *536.12 | *0.55 | *0.04 | 392.70 | 0.19 | 0.45 | *691.35 | *0.77 | *0.00 | 474.33 | 0.25 | 0.58 |
| Solyc02g084720.3 | 38.58 | 0.15 | 0.84 | 28.05 | −0.35 | 0.40 | *85.61 | *1.06 | *0.01 | 54.59 | 0.38 | 0.62 |
| Solyc07g015980.4 | 5.50 | −0.14 | 0.69 | 5.48 | −0.35 | 0.14 | *6.52 | *0.47 | *0.03 | 5.02 | −0.19 | 0.64 |
| Solyc08g065220.3 | 38.13 | −0.10 | 0.82 | *25.23 | *−0.72 | *0.00 | *61.65 | *0.52 | *0.05 | 41.09 | −0.09 | 0.83 |
| Solyc08g082620.3 | 8.05 | −0.38 | 0.40 | 8.64 | −0.52 | 0.15 | *21.37 | *1.18 | *0.00 | 9.69 | −0.11 | 0.85 |
| Solyc01g111330.4 | 15.03 | −0.22 | 0.77 | 15.81 | −0.54 | 0.25 | *41.36 | *1.38 | *0.00 | 19.04 | 0.12 | 0.88 |
| Solyc01g091620.3 | 42.14 | 0.21 | 0.55 | 28.05 | −0.38 | 0.14 | *54.96 | *0.51 | *0.05 | 40.51 | 0.06 | 0.88 |
| Solyc05g056250.3 | 23.75 | 0.30 | 0.31 | 16.47 | −0.35 | 0.16 | *32.17 | *0.67 | *0.00 | 21.88 | 0.06 | 0.89 |
| Solyc06g053510.3 | 17.94 | −0.81 | 0.36 | *11.53 | *−1.44 | *0.05 | *82.18 | *1.46 | *0.04 | 28.21 | −0.07 | 0.96 |
| Solyc08g069120.4 | 10.83 | −0.10 | 0.75 | 10.97 | −0.30 | 0.14 | 8.80 | −0.37 | 0.08 | *8.71 | *−0.47 | *0.03 |
| Solyc01g008960.3 | 78.88 | 0.03 | 0.95 | 51.21 | −0.55 | 0.05 | 110.52 | 0.41 | 0.20 | 79.87 | −0.06 | 0.91 |
| Solyc09g074240.1 | 23.37 | −0.38 | 0.28 | 27.94 | −0.25 | 0.37 | 21.13 | −0.58 | 0.05 | 28.96 | −0.04 | 0.93 |
| Solyc04g005040.1 | 205.44 | 0.13 | 0.86 | 269.93 | 0.61 | 0.20 | 225.60 | 0.09 | 0.98 | 425.60 | 0.99 | 0.07 |
| Solyc07g062700.3 | 316.45 | 0.76 | 0.07 | 300.79 | 0.78 | 0.06 | 287.85 | 0.40 | 0.47 | 376.93 | 0.79 | 0.10 |
| Solyc05g054040.4 | 6.79 | −0.39 | 0.10 | 7.96 | −0.35 | 0.13 | 10.28 | 0.41 | 0.07 | 6.32 | −0.43 | 0.10 |
| Solyc01g112000.4 | 181.14 | 0.08 | 0.90 | 240.71 | 0.61 | 0.10 | 177.21 | −0.20 | 0.80 | 325.67 | 0.68 | 0.12 |
| Solyc01g073710.2 | 30.41 | −0.12 | 0.87 | 35.41 | 0.25 | 0.54 | 31.13 | −0.25 | 0.77 | 58.35 | 0.72 | 0.17 |
| Solyc01g096000.2 | 16.24 | −0.43 | 0.19 | 18.38 | −0.52 | 0.08 | 20.28 | −0.11 | 0.90 | 15.85 | −0.47 | 0.19 |
| Solyc01g103490.3 | 22.79 | 0.38 | 0.33 | 20.57 | 0.21 | 0.49 | 23.48 | 0.28 | 0.55 | 26.96 | 0.48 | 0.26 |
| Solyc12g088210.3 | 7.73 | 0.02 | 0.96 | 8.05 | −0.20 | 0.31 | 7.91 | 0.13 | 0.72 | 9.08 | 0.28 | 0.29 |
| Solyc01g096760.3 | 11.62 | 0.42 | 0.06 | 10.28 | 0.01 | 0.95 | 9.07 | 0.12 | 0.79 | 9.90 | 0.24 | 0.47 |
| Solyc03g112620.4 | 21.75 | −0.18 | 0.78 | 22.46 | −0.10 | 0.78 | 29.46 | 0.00 | 1.00 | 37.41 | 0.36 | 0.57 |
| Solyc04g080190.3 | 10.64 | 0.12 | 0.71 | 8.59 | −0.40 | 0.06 | 10.01 | 0.13 | 0.74 | 9.04 | −0.19 | 0.59 |
| Solyc04g150161.1 | 8494.18 | 0.25 | 0.50 | 5939.81 | −0.09 | 0.71 | 11633.98 | 0.34 | 0.31 | 8920.36 | 0.22 | 0.64 |
| Solyc08g029110.3 | 9.63 | −0.13 | 0.69 | 8.72 | −0.36 | 0.11 | 10.88 | 0.00 | 1.00 | 9.89 | −0.15 | 0.70 |
| Solyc03g007240.3 | 30.53 | 0.82 | 0.14 | 18.78 | 0.04 | 0.94 | 18.78 | −0.04 | 1.00 | 23.40 | 0.30 | 0.74 |
| Solyc02g068130.4 | 11.54 | −0.05 | 0.89 | 10.39 | −0.33 | 0.13 | 13.53 | 0.25 | 0.36 | 11.20 | −0.12 | 0.76 |
| Solyc05g007950.4 | 7.41 | 1.13 | 0.16 | 5.20 | 0.63 | 0.37 | 7.60 | 0.99 | 0.24 | 5.44 | 0.40 | 0.76 |
| Solyc01g014840.3 | 23.14 | −0.65 | 0.15 | 33.04 | −0.09 | 0.82 | 28.68 | −0.40 | 0.48 | 43.86 | 0.22 | 0.76 |
| Solyc02g067860.4 | 19.50 | 0.20 | 0.63 | 18.29 | 0.11 | 0.68 | 15.39 | −0.34 | 0.33 | 21.07 | 0.15 | 0.76 |
| Solyc07g064130.2 | 1336.69 | 0.09 | 0.82 | 1210.17 | 0.04 | 0.88 | 1313.88 | −0.11 | 0.87 | 1527.39 | 0.12 | 0.78 |
| Solyc06g083300.4 | 60.24 | 0.24 | 0.51 | 39.22 | −0.40 | 0.13 | 53.85 | −0.07 | 0.96 | 52.29 | −0.12 | 0.79 |
| Solyc11g006990.2 | 37.71 | −0.07 | 0.95 | 26.24 | −0.61 | 0.28 | 61.26 | 0.32 | 0.80 | 59.68 | 0.27 | 0.79 |
| Solyc02g092750.3 | 30.63 | 0.25 | 0.65 | 22.12 | −0.28 | 0.42 | 29.49 | −0.04 | 0.99 | 27.26 | −0.15 | 0.80 |
| Solyc09g098520.4 | 10.59 | 0.03 | 0.95 | 9.71 | −0.27 | 0.21 | 12.74 | 0.26 | 0.32 | 10.57 | −0.09 | 0.81 |
| Solyc05g056340.3 | 10.77 | −0.03 | 0.92 | 11.31 | −0.17 | 0.31 | 10.52 | 0.03 | 0.98 | 10.11 | −0.07 | 0.81 |
| Solyc06g008340.4 | 14.01 | 0.17 | 0.65 | 12.44 | −0.14 | 0.56 | 11.85 | −0.22 | 0.56 | 13.20 | −0.09 | 0.83 |
| Solyc10g005510.3 | 37.75 | −0.20 | 0.62 | 33.64 | −0.33 | 0.22 | 55.99 | 0.32 | 0.34 | 48.72 | 0.10 | 0.83 |
| Solyc01g090710.3 | 4.43 | −0.04 | 0.93 | 5.28 | 0.28 | 0.30 | 3.86 | 0.13 | 0.86 | 4.81 | 0.10 | 0.83 |
| Solyc12g005140.2 | 5.98 | 0.05 | 0.85 | 6.50 | 0.01 | 0.93 | 5.97 | 0.21 | 0.26 | 5.41 | −0.06 | 0.84 |
| Solyc11g040110.2 | 14.63 | 0.47 | 0.10 | 11.60 | 0.05 | 0.86 | 12.40 | 0.24 | 0.54 | 11.72 | 0.09 | 0.85 |
| Solyc04g078760.4 | 16.74 | 0.03 | 0.94 | 15.35 | −0.24 | 0.29 | 15.03 | −0.11 | 0.84 | 15.71 | −0.07 | 0.86 |
| Solyc01g100870.3 | 94.89 | 0.38 | 0.19 | 56.29 | −0.37 | 0.16 | 94.93 | 0.27 | 0.42 | 82.00 | 0.06 | 0.89 |
| Solyc04g078390.3 | 149.59 | 0.40 | 0.25 | 98.27 | −0.27 | 0.35 | 126.94 | 0.11 | 0.92 | 123.39 | 0.06 | 0.91 |
| Solyc08g075860.3 | 37.50 | −0.24 | 0.51 | 30.90 | −0.46 | 0.08 | 41.80 | −0.19 | 0.67 | 46.15 | −0.05 | 0.92 |
| Solyc03g112080.1 | 12.86 | 0.68 | 0.25 | 13.16 | 0.70 | 0.17 | 7.22 | −0.05 | 0.99 | 7.72 | 0.09 | 0.92 |
| Solyc09g092380.3 | 197.28 | 0.22 | 0.49 | 124.11 | −0.39 | 0.10 | 265.91 | 0.40 | 0.11 | 206.20 | 0.03 | 0.93 |
| Solyc02g069100.4 | 25.23 | −0.02 | 0.97 | 18.99 | −0.51 | 0.10 | 27.68 | 0.10 | 0.93 | 26.48 | 0.04 | 0.93 |
| Solyc01g087900.3 | 29.53 | 0.20 | 0.57 | 21.03 | −0.32 | 0.21 | 34.68 | 0.31 | 0.32 | 29.00 | 0.04 | 0.94 |
| Solyc11g012705.2 | 11.78 | −0.48 | 0.70 | 17.72 | 0.17 | 0.82 | 14.85 | −0.35 | 0.87 | 17.76 | −0.06 | 0.97 |
| Solyc05g050200.4 | 38.72 | 0.02 | 0.96 | 27.78 | −0.50 | 0.09 | 42.60 | 0.04 | 0.99 | 41.59 | 0.02 | 0.98 |
| Solyc06g073150.3 | 10.08 | 0.10 | 0.77 | 8.86 | −0.37 | 0.08 | 11.16 | 0.38 | 0.06 | 9.49 | −0.01 | 0.99 |
| Solyc06g082380.3 | 10.16 | 0.27 | 0.40 | 9.65 | 0.02 | 0.94 | 8.92 | 0.15 | 0.75 | 9.03 | 0.00 | 0.99 |
| Solyc02g089070.3 | 31.06 | 0.23 | 0.55 | 20.59 | −0.40 | 0.15 | 33.45 | 0.25 | 0.51 | 28.02 | 0.00 | 1.00 |
| Solyc02g087530.2 | 4.18 | 0.42 | 0.84 | 7.35 | 1.74 | 0.16 | 3.39 | 0.27 | 0.98 | 3.91 | 0.69 | NA |
| Solyc01g095995.2 | Not | | | Not | | | Not | | | Not | | |
| | detected | | | detected | | | detected | | | detected | | |
| Solyc08g083120.2 | Not | | | Not | | | Not | | | Not | | |
| | detected | | | detected | | | detected | | | detected | | |

### Example 6—the Analysis of Meloidogyne Incognita—Tomato Interactome Yielded Several Discoveries

RKN uses different effectors to target the same protein to sustain host protein targeting during various stage of infection. For example, Solyc01g097520.4 (SEQ ID NO: 12) is targeted by 7 different effectors originated form the nematode subventral and dorsal gland cells, which are active during early and sedentary stages of infection, respectively. Similarly, Solyc02g067860.4 (SEQ ID NO: 25) is targeted by 5 effectors originated form the nematode subventral and dorsal gland cells. Thus, nematode use various effectors to target common host proteins to maintain its ability to parasite host plants in case of loss or rapid selection against individual effectors. Proteins targeted by more than one nematode effector are therefore considered genuine targets for enhancing tomato resistance to RKN.

We discovered that nematode effectors individually target homologous proteins with the same function. For example, we discovered that effector 8 targets two tomato cytosolic glutamine synthetases (Solyc04g014510.3 (SEQ ID NO: 42) and Solyc11g011380.2 (SEQ ID NO: 96)) as well as two cathepsin B-like cysteine proteinases (Solyc02g069100.4 (SEQ ID NO: 28) and Solyc04g078540.4 (SEQ ID NO: 48)). Similarly, effector 2 target two proteins coding for aldose 1-epimerase family proteins (Solyc02g067860.4 (SEQ ID NO: 25) and Solyc02g087770.3 (SEQ ID NO: 33)).

We discovered that nematode effectors collectively target homologous proteins with the same function. For example, effectors 7 and 8 target two S-adenosyl-L-methionine synthetase homologs (Solyc01g101060.4 (SEQ ID NO: 14) and Solyc09g008280.2 (SEQ ID NO: 83)), effectors 2 and 8 target two expansin-like protein homologs (Solyc01g112000.1 (SEQ ID NO: 22) and Solyc02g081210.3 (SEQ ID NO: 30)), effectors 2 and 5 target pathogenesis-related thaumatin homologs (Solyc01g111330.4 (SEQ ID NO: 19) and Solyc11g066130.1 (SEQ ID NO: 99)), effector 6 and 7 target two SnRK1-interacting protein 1 homologs (Solyc11g040110.2 (SEQ ID NO: 98) and Solyc11g012705.2 (SEQ ID NO: 97)), and effectors 2 and 7 target two SNF1 kinase complex anchoring protein homologs (Solyc02g067030.4 (SEQ ID NO: 24) and Solyc12g099970.2 (SEQ ID NO: 103)).

We discovered that nematode effectors collectively target different proteins involved in the same biological processes or pathways. For example, effectors 2, 4, 7, 8, and 11 target different proteins involved in glycolysis (Solyc02g087770.3 (SEQ ID NO: 33), Solyc12g095880.2 (SEQ ID NO: 102), Solyc10g005510.3 (SEQ ID NO: 91), Solyc03g111010.4 (SEQ ID NO: 38), and Solyc01g090710.3 (SEQ ID NO: 6)), effectors 1, 2, 8, 9, and 11, target different proteins involved in Immunity (Solyc01g097520.4 (SEQ ID NO: 12), Solyc01g103490.3 (SEQ ID NO: 15), Solyc06g082380.3 (SEQ ID NO: 66), Solyc08g075860.3 (SEQ ID NO: 80), and Solyc04g080960.4 (SEQ ID NO: 51)), effectors 2, 3, 8, and 9 target different proteins involved in vesicle trafficking (Solyc06g008340.4 (SEQ ID NO: 59), Solyc01g111520.3 (SEQ ID NO: 21), Solyc01g100870.3 (SEQ ID NO: 13), and Solyc06g050770.3 (SEQ ID NO: 60)), effectors 6 and 11 target different proteins involved in polyamine biosynthesis (Solyc03g007240.3 (SEQ ID NO: 36), Solyc04g082030.1 (SEQ ID NO: 52), Solyc06g053510.3 (SEQ ID NO: 61), and Solyc07g015980.4 (SEQ ID NO: 71)). The finding that effector 11 targets three enzymes involved in polyamine biosynthesis is interesting and shows how a single effector can efficiently manipulate the whole pathway by targeting various enzymes involved in key steps in the biosynthesis.

We compared Arabidopsis orthologs of our tomato proteins targeted by nematode effectors with Arabidopsis proteins targeted by effectors from the bacterial pathogen Pseudomonas syringae, the oomycete pathogen Hyaloperonospora arabidopsidis, and the ascomycete pathogen Golovinomyces orontii (Mukhtar et al. 2011; Weßling et al. 2014). Interestingly, we found that Arabidopsis ortholog (AT1G22920) of the tomato protein Solyc06g073150.3, which is targeted by effector 5, is also targeted by effectors from P. syringae, H. arabidopsidis, and H. arabidopsidis. Similarly, we found that Arabidopsis ortholog (AT2G38750) of the tomato protein Solyc01g097520.4 (SEQ ID NO: 12), which is targeted by 7 nematode effectors, is also targeted by effectors from H. arabidopsidis. This indicates that distantly-related plant pathogens evolved independently to target common host proteins to modulate host physiology and increase pathogen fitness.

We compared tobacco (Nicotiana benthamiana) orthologs of our tomato proteins targeted by nematode effectors with N. benthamiana proteins targeted by effectors from the potato blight pathogen Phytophthora infestans (Petre et al., 2021). Remarkably, we found that homologs of 32 tomato proteins (Solyc04g078540.4 (SEQ ID NO: 48), Solyc01g111450.3 (SEQ ID NO: 20), Solyc05g054040.4 (SEQ ID NO: 104), Solyc11g011910.2 (SEQ ID NO: 105), Solyc04g014570.3 (SEQ ID NO: 43), Solyc01g105070.3 (SEQ ID NO: 16), Solyc09g008280.2 (SEQ ID NO: 83), Solyc01g101060.4 (SEQ ID NO: 14), Solyc12g099000.3 (SEQ ID NO: 115), Solyc02g087770.3 (SEQ ID NO: 33), Solyc08g065220.3 (SEQ ID NO: 78), Solyc02g084720.3 (SEQ ID NO: 31), Solyc09g009020.3 (SEQ ID NO: 84), Solyc06g050770.3 (SEQ ID NO: 60), Solyc07g065840.2 (SEQ ID NO: 75), Solyc04g014510.3 (SEQ ID NO: 42), Solyc11g011380.2 (SEQ ID NO: 96), Solyc09g097960.3 (SEQ ID NO: 89), Solyc10g006480.2 (SEQ ID NO: 117), Solyc11g005670.2 (SEQ ID NO: 113), Solyc07g064130.2 (SEQ ID NO: 73), Solyc05g014470.3 (SEQ ID NO: 108), Solyc03g111010.4 (SEQ ID NO: 38), Solyc10g005510.3 (SEQ ID NO: 91), Solyc09g092390.2 (SEQ ID NO: 106), Solyc09g092380.3 (SEQ ID NO: 88), Solyc01g105340.4 (SEQ ID NO: 17), Solyc02g069090.3 (SEQ ID NO: 27), Solyc02g069100.4 (SEQ ID NO: 28), Solyc08g082620.3 (SEQ ID NO: 82), Solyc01g111520.3 (SEQ ID NO: 21), and Solyc01g090710.3 (SEQ ID NO: 6)) are also targeted by effectors from P. infestans. This discovery further confirms that biotrophic pathogens such as RKN and potato blight pathogen target and manipulate a common set of host proteins involved in molecular processes and signaling pathways required for successful infection.

Based on these analyses, we regard genes coding for tomato proteins targeted by multiple nematode effectors or targeted by multiple effectors from distinct pathogens as excellent gene candidates to improve tomato resistance to M. incognita. This is consistent with the finding that mutants of genes encoding host proteins repeatedly targeted by multiple effectors from individual pathogen or from different pathogens generally exhibit disease phenotypes (Mukhtar et al. 2011; Weßling et al. 2014).

We consider tomato proteins targeted uniquely by M. incognita effectors as pathogen-specific host targets, which may be required to support nematode life style and feeding habit. These are also considered as bona fide targets to improve tomato resistance to M. incognita.

Of the 11 M. incognita effectors investigated here, 7 (effector #2, 3, 5, 6, 7, 9, and 11) have orthologs in the northern root-knot nematode Meloidogyne hapla, which infects almost all temperate vegetables and dramatically reduces yield. These effectors may be part of RKN core effectors, and hence tomato genes encoding proteins interacting with these 7 effectors could be used to improve plant resistance against both M. incognita and M. hapla.

Because of the ability of M. incognita to infect more than 2000 plant species, plant proteins targeted by M. incognita effectors may be conserved across plants.

### Example 7—Virus-Induced Gene Silencing (VIGS)

To further validate the involvement of the resistance candidate genes in establishing tomato interaction of M. incognita, we used Virus-induced gene silencing (VIGS) approach to partially silence a set of 7 genes involved in different biological processes. These 7 genes include Solyc09g092380 (SEQ ID NO: 217), Solyc04g014570 (SEQ ID NO: 172), Solyc01g111520 (SEQ ID NO: 150), Solyc08g069120 (SEQ ID NO: 208), Solyc02g081210 (SEQ ID NO: 159), Solyc05g056340 (SEQ ID NO: 187), and Solyc01g100870 (SEQ ID NO: 142). We used Tobacco rattle virus (TRV)-RNA1 (TRV1) and TRV-RNA2 (TRV2) as a VIGS vectors to induce gene silencing (Ryu et al., 2004). We cloned a fragment of about 350 nucleotides (nucleotides 1 to 311 of SEQ ID NO: 217 (Solyc09g092380); nucleotides 18 to 358 of SEQ ID NO: 172 (Solyc04g014570); nucleotides 174 to 469 of SEQ ID NO: 150 (Solyc01g111520); nucleotides 436 to 730 of SEQ ID NO: 208 (Solyc08g069120); nucleotides 1 to 280 of SEQ ID NO: 159 (Solyc02g081210); nucleotides 1825 to 2132 of SEQ ID NO: 187 (Solyc05g056340); and nucleotides 305 to 632 of SEQ ID NO: 142 (Solyc01g100870)) of each gene in the pYL156 (TRV RNA2) vector using EcoRI and BamHI restriction sites. Cloned fragments were verified by DNA sequencing. The TRV RNA2 vectors containing gene fragments were transformed into Agrobacterium tumefaciens (strain LBA4404). Empty TRV-RNA1 vector, which contains two viral replication proteins, a movement protein, and a seed transmission factor was also transformed to A. tumefaciens strain LBA4404. Overnight Agrobacterium cultures were harvested and suspended into an inoculation buffer (10 mM MgCl2, 150 μM acetosyringone, and 10 mM MES pH 5.6) to a final OD600 of 1.0. Agrobacterium strains containing each of the constructed TRV2 vectors were mixed separately with the Agrobacterium strain containing the empty TRV1 vector in a 1:1 ratio. The mix were used to infiltrate the roots of 7-day-old seedling to tomato cultivar Heinz, which is susceptible to the root knot nematode M. incognita. As a control treatment, Heinz tomato seedlings were infiltrated with a mixture of Agrobacterium cultures containing the empty TRV1 vector and the empty TRV2 vector in a 1:1 ratio. The vacuum infiltration treatment was conducted three time each for one minute. At least 15 seedlings per gene were infiltrated.

The agro-infiltrated seedlings were planted in Peat/Bark Based Growing Soil, and kept in controlled phytotron at 26° C. under 16-h-light/8-h-dark conditions. Ten days after planting, each seedling was inoculated with about 500 second-stage juveniles of M. incognita. Approximately 3 weeks after nematode inoculation, the number of nematode-inducted galls on the root of each plant was counted and used to determine plant susceptibility levels. VIGS-induced gene silencing of Solyc09g092380 and Solyc04g014570 significantly increased plant susceptibility to M. incognita. In contrast, VIGS-induced gene silencing of Solyc01g111520, Solyc08g069120, Solyc02g081210, Solyc05g056340, and Solyc01g100870 significantly reduced plant susceptibility to M. incognita. These functional data revealed a key role of the reported resistance candidate genes in tomato-M. incognita interactions and these genes can be used to generate resistance to root-knot nematode in tomato and other plants via genome editing and the generation of transgenic plants.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

Abrahamian, P., Sharma, A., Jones, J. B., and Vallad, G. E. 2021. Dynamics and spread of bacterial spot epidemics in tomato transplants grown for field production. Plant Disease 105:566-575.
Bellafiore S, Shen, Z. X., Rosso, M. N., Abad, P., Shih, P., and Briggs, S. P. 2008. direct identification of the Meloidogyne incognita secretome reveals proteins with host cell reprogramming potential. PLOS Pathog, 4 (10), e1000192.
Castagnone-Sereno, P., 2006. Genetic variability and adaptive evolution in parthenogenetic root-knot nematodes. Heredity, 96 (4), pp. 282-289.
Elling, A. A. 2013. Major Emerging Problems with Minor Meloidogyne Species. Phytopathology 103:1092-1102.
Eves-van den Akker, S., Lilley, C. J., Jones, J. T. and Urwin, P. E., 2014. Identification and characterisation of a hyper-variable apoplastic effector gene family of the potato cyst nematodes. PLOS pathogens, 10 (9), p.e1004391
Fosu-Nyarko, J. and Jones, M. G., 2016. Advances in understanding the molecular mechanisms of root lesion nematode host interactions. Annual Review of Phytopathology, 54, pp. 253-278.
Gardner M, Dhroso A, Johnson N, Davis E L, Baum T J, Korkin D, Mitchum M G. (2018) Novel global effector mining from the transcriptome of early life stages of the soybean cyst nematode Heterodera glycines. Sci Rep. 8 (1): 2505.
Gheysen, G. and Mitchum, M. G., 2019. Phytoparasitic nematode control of plant hormone pathways. Plant physiology, 179 (4), pp. 1212-1226.
González-Fuente, M., Carrère, S., Monachello, D., Marsella, B. G., Cazalé, A. C., Zischek, C., Mitra, R. M., Rezé, N., Cottret, L., Mukhtar, M. S. and Lurin, C., 2020. EffectorK, a comprehensive resource to mine for Ralstonia, Xanthomonas, and other published effector interactors in the Arabidopsis proteome. Molecular plant pathology, 21 (10), pp. 1257-1270.
Goverse A., Smant G. (2014) The activation and suppression of plant innate immunity by parasitic nematodes. Annu Rev Phytopathol, 52:243-265.
Hafez L S and Sundararaj P. 2009. Soil fumigation a tool for sugar beet cyst nematode management. University of Idaho Extension: CIS 1163.
Hewezi T., Baum T. J. 2013. Manipulation of plant cells by cyst and root-knot nematode effectorsMol Plant Microbe Interact, 26, pp. 9-16.
Hewezi, T., 2015. Cellular signaling pathways and posttranslational modifications mediated by nematode effector proteins. Plant physiology, 169 (2), pp. 1018-1026.
Hewezi, T., 2020. Epigenetic mechanisms in nematode-plant interactions. Annual review of phytopathology, 58, pp. 119-138.
Hewezi, T., Howe, P., Maier, T. R., Hussey, R. S., Mitchum, M. G., Davis, E. L. and Baum, T. J., 2008. Cellulose binding protein from the parasitic nematode Heterodera schachtii interacts with Arabidopsis pectin methylesterase: cooperative cell wall modification during parasitism. The Plant Cell, 20 (11), pp. 3080-3093.
Hewezi, T., Howe, P. J., Maier, T. R., Hussey, R. S., Mitchum, M. G., Davis, E. L. and Baum, T. J., 2010. Arabidopsis spermidine synthase is targeted by an effector protein of the cyst nematode Heterodera schachtii. Plant physiology, 152 (2), pp. 968-984.
Hewezi, T., Juvale, P. S., Piya, S., Maier, T. R., Rambani, A., Rice, J. H., Mitchum, M. G., Davis, E. L., Hussey, R. S. and Baum, T. J., 2015. The cyst nematode effector protein 10A07 targets and recruits host posttranslational machinery to mediate its nuclear trafficking and to promote parasitism in Arabidopsis. The Plant Cell, 27 (3), pp. 891-907.
Hewezi, T., Piya, S., Qi, M., Balasubramaniam, M., Rice, J. H. and Baum, T. J., 2016. Arabidopsis miR827 mediates post-transcriptional gene silencing of its ubiquitin E3 ligase target gene in the syncytium of the cyst nematode Heterodera schachtii to enhance susceptibility. The Plant Journal, 88 (2), pp. 179-192.
Hu, Y. Hewezi T. 2018. Nematode-secreted peptides and host factor mimicry. J Exp Bot, 69, pp. 2866-2868.
Hussey, R. S. 1989. Disease-inducing secretions of plant parasitic nematodes. Annu. Rev. Phytopathol. 27:123-141.
Hussey, R. S., and Mims C. W. 1990. Ultrastructure of esophageal glands and their secretory granules in the root-knot nematode Meloidogyne incognita. Protoplasma 156:9-18.
Jones J T, Haegeman A, Danchin E G, Gaur H S, Helder J, Jones M G, Kikuchi T, Manzanilla-López R, Palomares-Rius J E, Wesemael W M, Perry R N. 2013. Top 10 plant-parasitic nematodes in molecular plant pathology. Mol Plant Pathol. 14 (9): 946-961.
Maier, T. R., Hewezi, T., Peng, J. and Baum, T. J., 2013. Isolation of whole esophageal gland cells from plant-parasitic nematodes for transcriptome analyses and effector identification. Molecular Plant-Microbe Interactions, 26 (1), pp. 31-35.
Mejias, J., Truong, N. M., Abad, P., Favery, B. and Quentin, M., 2019. Plant proteins and processes targeted by parasitic nematode effectors. Frontiers in plant science, 10, p. 970.
Mukhtar, M. S., Carvunis, A. R., Dreze, M., Epple, P., Steinbrenner, J., Moore, J., Tasan, M., Galli, M., Hao, T., Nishimura, M. T. and Pevzner, S. J., 2011. Independently evolved virulence effectors converge onto hubs in a plant immune system network. science, 333 (6042), pp. 596-601.
Noling, J. 2016. Nematode management in tomatoes, peppers, and eggplant. University of Florida IFAS extension, ENY-032.
Petre B, Contreras M P, Bozkurt T O, Schattat M H, Sklenar J, Schornack S, Abd-El-Haliem A, Castells-Graells R, Lozano-Duran R, Dagdas Y F, Menke FLH, Jones AME, Vossen J H, Robatzek S, Kamoun S, Win J. (2021). Host-interactor screens of Phytophthora infestans RXLR proteins reveal vesicle trafficking as a major effector-targeted process. Plant Cell. doi: 10.1093/plcell/koab069.
Petre, B., Saunders, D. G., Sklenar, J., Lorrain, C., Krasileva, K. V., Win, J., Duplessis, S. and Kamoun, S., 2016. Heterologous expression screens in Nicotiana benthamiana identify a candidate effector of the wheat yellow rust pathogen that associates with processing bodies. PloS one, 11 (2), p.e0149035.
Petre, B., Saunders, D. G., Sklenar, J., Lorrain, C., Win, J., Duplessis, S. and Kamoun, S., 2015. Candidate effector proteins of the rust pathogen Melampsora larici-populina target diverse plant cell compartments. Molecular Plant-Microbe Interactions, 28 (6), pp. 689-700.
Regmi, H., and Desaeger, J. 2020. Integrated management of root-knot nematode (Meloidogyne spp.) in Florida tomatoes combining host resistance and nematicides. Crop Prot 134
Rutter, W. B., Hewezi, T., Abubucker, S., Maier, T. R., Huang, G., Mitreva, M., Hussey, R. S. and Baum, T. J., 2014. Mining novel effector proteins from the esophageal gland cells of Meloidogyne incognita. Molecular Plant-Microbe Interactions, 27 (9), pp. 965-974.
Ryu, C. M., Anand, A., Kang, L. and Mysore, K. S., 2004. Agrodrench: a novel and effective agroinoculation method for virus-induced gene silencing in roots and diverse Solanaceous species. The Plant Journal, 40 (2), pp. 322-331.
Siddique S, Grundler F. M. (2018) Parasitic nematodes manipulate plant development to establish feeding sites. Curr Opin Microbiol, 46:102-108.
Smith, P. G. 1944. Embryo culture of a tomato species hybrid. Proceedings of the American Society for Horticultural Science 44:413-416.
Vieira P, Gleason C. 2019Plant-parasitic nematode effectors-insights into their diversity and new tools for their identification. Curr Opin Plant Biol. 50:37-43.
Vieira, P., Maier, T. R., Eves-van den Akker, S., Howe, D. K., Zasada, I., Baum, T. J., Eisenback, J. D. and Kamo, K., 2018. Identification of candidate effector genes of Pratylenchus penetrans. Molecular plant pathology, 19 (8), pp. 1887-1907.
Weßling, R., Epple, P., Altmann, S., He, Y., Yang, L., Henz, S. R., McDonald, N., Wiley, K., Bader, K. C., Gläßer, C. and Mukhtar, M. S., 2014. Convergent targeting of a common host protein-network by pathogen effectors from three kingdoms of life. Cell host & microbe, 16 (3), pp. 364-375.

I claim:

1. A plant cell comprising one or more underexpressed, overexpressed, silenced, inactivated, or overexpressed and inactivated gene, wherein the underexpressed, overexpressed, silenced, inactivated, or overexpressed and inactivated gene comprises a nucleic acid sequence encoding a protein selected from SEQ ID NOs: 1 to 118 or homologs thereof.

2. The plant cell of claim 1, wherein the plant cell comprises:
i) in the endogenous promoter one or more mutations that increase the expression of the gene;
ii) a heterologous nucleic acid comprising the gene;
iii) one or more mutations in the endogenous promoter that significantly reduces or abolishes the expression of the gene;
iv) an inhibitory oligonucleotide that significantly reduces, silences, or abolishes the expression of the gene;
v) a deletion of the endogenous gene;
vi) the overexpressed, underexpressed, or inactivated gene integrated into the germplasm of the plant cell or the plant;
vii) the overexpressed, underexpressed, or inactivated gene integrated into the germplasm of the plant cell or the plant; or
viii) the overexpressed gene under endogenous promoter and/or an exogenous promoter.

3. The plant cell of claim 1, wherein the protein encoded by the overexpressed, inactivated, silenced, or underexpressed gene cannot interact with one or more root-knot nematode (RKN) effector proteins of SEQ ID NOs: 119 to 129 or homologs thereof.

4. The plant cell of claim 3, wherein the protein encoded by the inactivated, silenced, or underexpressed gene cannot interact with one or more root-knot nematode (RKN) effector proteins of SEQ ID NOs: 13, 21, 30, 58, 79 or homologs thereof.

5. The plant cell of claim 1, wherein the plant cell is in a plant part.

6. The plant cell of claim 5, wherein the plant part is a root.

7. The plant cell of claim 1, wherein the plant is a tomato plant or other RKN-host plants.

8. A method for identifying a gene that induces root-knot nematode (RKN) resistance when overexpressed, underexpressed, silenced, inactivated, or overexpressed and inactivated in a plant cell or a plant, the method comprising the steps of: (a) overexpressing or underexpressing a gene in a plant cell or a plant, compared to the expression in a plant cell or a plant known to be susceptible to RKN, silencing a gene in a plant cell or a plant in a plant cell or a plant known to be susceptible to RKN, or inactivating a gene in a plant cell or a plant known to be resistant to RKN, (b) testing the RKN resistance in the plant cell or the plant comprising the overexpressed, underexpressed, silenced, inactivated, or the overexpressed and inactivated gene, and (c) identifying the gene that induces resistance in the plant cell or the plant when overexpressed, underexpressed, silenced, inactivated, or both overexpressed and inactivated.

9. The method of claim 8, wherein the overexpressed, underexpressed, silenced or inactivated gene comprises a nucleic acid sequence of any one of SEQ ID NOs: 130 to 247 or an amino acid sequence of any one of SEQ ID NOs: 1 to 118 or homologs thereof, or a gene comprising a nucleic acid sequence of any one of SEQ ID NOs: 130 to 247 or an amino acid sequence of any one of SEQ ID NOs: 1 to 118 or homologs thereof is overexpressed, underexpressed, silenced, or inactivated.

10. The method of claim 9, wherein the underexpressed, silenced or inactivated gene comprises a nucleic acid sequence of any one of SEQ ID NOs: 142, 150, 159, 187, or 208 or an amino acid sequence of any one of SEQ ID NOs: 13, 21, 30, 58, 79 or homologs thereof, or a gene comprising a nucleic acid sequence of any one of SEQ ID NOs: 142, 150, 159, 187, or 208 or an amino acid sequence of any one of SEQ ID NOs: 13, 21, 30, 58, 79 or homologs thereof is underexpressed, silenced, or inactivated.

11. The method of claim 8, wherein the plant cell or the plant is a tomato plant cell or a tomato plant or other RKN host plants.

12. The method of claim 8, comprising:
i) introducing in the endogenous promoter of the gene one or more mutations that increase the expression of the gene;
ii) introducing into the plant cell or the plant a heterologous nucleic acid comprising the gene;
iii) introducing one or more mutations in the endogenous promoter and/or the coding sequence of the gene that significantly reduces or abolishes the expression of the gene;
iv) introducing into the plant cell or the plant an inhibitory oligonucleotide that significantly reduces, silences, or abolishes the expression of the gene;
v) deleting the endogenous gene in the plant;
vi) using molecular markers or other traditional breeding methods to integrate the overexpressed, underexpressed, or inactivated gene in the germplasm of the plant cell or the plant;
vii) the overexpressed, underexpressed, or inactivated gene integrated into the germplasm of the plant cell or the plant; or
viii) the overexpressed gene under endogenous promoter and/or an exogenous promoter.

13. The method of claim 12, wherein deleting the endogenous gene in the plant comprises CRISPR/Cas mediated mutations of the gene.

14. A method of inhibiting the interaction of one or more plant proteins with one or more root-knot nematode (RKN) effector proteins, the method comprising the steps of:
a) overexpressing, underexpressing, silencing, inactivating, or overexpressing and inactivating the one or more plant proteins in a plant cell.

15. The method of claim 14, wherein the overexpressed or underexpressed one or more plant proteins has an expression difference of at least 50%.

16. The method of claim 14, wherein the plant is a tomato plant.

17. The method of claim 14, wherein the plant cell is a root cell.

18. The method of claim 14, wherein the overexpressed, underexpressed, inactivated, or overexpressed and inactivated one or proteins is created by mutating the nucleotides encoding about 1 to about 30, about 1 to about 20, about 1 to about 10, about 1 to about 5, or about 1 to about 2 amino acids of the protein.
